(19) United States
(12) Patent Application Publication
(10) Pub. No.: US 2026/0176588 A1
(43) Pub. Date: Jun. 25, 2026

(54) ENGINEERING CELL LINES CAPABLE OF PROLIFERATION IN GROWTH FACTOR FREE MEDIA FORMULATIONS

(71) Applicant: Upside Foods, Inc., Emeryville, CA (US)

(72) Inventors: Sukhdeep Singh DHADWAR, El Cerrito, CA (US); Rachel Michele SCHUMAKER, Emeryville, CA (US)

(21) Appl. No.: 19/549,587

(22) Filed: Feb. 25, 2026

Related U.S. Application Data
(63) Continuation of application No. 18/331,092, filed on Jun. 7, 2023, now Pat. No. 12,584,109.
(60) Provisional application No. 63/349,865, filed on Jun. 7, 2022.

(51) Int. Cl. C12N 5/077; A23L 13/00; C07K 14/49; C07K 14/50; C07K 14/65; C12N 9/12; C12N 15/85

(52) U.S. Cl. CPC C12N 5/0658; A23L 13/00; C07K 14/49; C07K 14/50; C07K 14/65; C12N 9/1241; C12N 15/85; C12N 2800/107

(57) ABSTRACT

Provided herein are methods of engineering a cell line for reduced dependence on exogenous growth factor(s). In some embodiments, the method includes introducing into a cell one or more of: a polynucleotide comprising a coding sequence of a growth factor ligand; a polynucleotide comprising a coding sequence of a growth factor receptor; or a polynucleotide comprising a coding sequence of an activated downstream growth factor target, and culturing the cells in a cultivation infrastructure.

### 1. CROSS-REFERENCE TO RELATED APPLICATIONS

This application is a continuation of U.S. patent application Ser. No. 18/331,092 filed on Jun. 7, 2023 which claims the benefit of U.S. Provisional Application No. 63/349,865 filed Jun. 7, 2022 which are hereby incorporated in their entirety by reference.

### 2. SEQUENCE LISTING

The instant application contains a Sequence Listing which has been submitted herewith via Patent Center and is hereby incorporated by reference in its entirety. Said XML copy, created on Feb. 25, 2026, is named 382790010U3_SL.xml, and is 252,412 bytes in size.

### 3. BACKGROUND

The mass production of cells remains limited by several factors, thus limiting final yields. Mass production of cells finds several downstream applications. For example, foods formulated from metazoan cells, cultured in vitro, have prospective advantages over their corporal-derived animal counterparts, including improved nutrition and safety. Production of these products have been projected to require fewer resources, convert biomass at a higher caloric efficiency and result in reduced environmental impacts relative to conventional in vivo methods. Together, metazoan cells, and their extracellular products, constitute a biomass that can potentially be harvested from a cultivation infrastructure for formulation of cell-based food products, such as cultured meat.

However, mass production of cells originating from cultured metazoan cells remains limited by several factors, for example, by the maximum culture density that can be conventionally achieved and the requirement for supplemented proteins, such as growth factors, which support the productivity of the cultivation process, thus limiting final yields. Provided herein are compositions and methods that address this and other related needs.

### 4. SUMMARY

This disclosure is based in part on the finding that introducing a polynucleotide comprising a coding sequence of a growth factor ligand and/or a coding sequence of a growth factor receptor into a cell line results in the cell line's reduced reliance on exogenous growth factors. For example, the Applicant found that introducing a polynucleotide comprising a coding sequence of a growth factor ligand and/or a coding sequence of a growth factor receptor into a cell line results in maintenance of cell proliferation rates similar to controls and an increase in the number of non-adherent cells (i.e., cells from anchorage-dependent growth) all while cells are grown in the absence of one or more exogenous growth factors (see FIG. 1).

Overall, this work demonstrated the ability to engineer cells to have reduced dependence on exogenous growth factors without compromising the cell's ability to proliferate or differentiate into myogenic cells. These findings are important because manufacturing cells suitable for consumption requires vast amounts of exogenous growth factors, which is both cost and time prohibitive (when factoring in supply chain). The engineered cells provided herein supply their own source of growth factor signaling-thereby bypassing or at least reducing the need to supplement the cultures with exogenous growth factors. Moreover, by promoting anchorage independent growth, which is currently essential for manufacturing cell based meats suitable for consumption, the engineered cell lines provided herein increase the efficiency by which cell based meats suitable for consumption can be produced.

In one aspect, this disclosure features a method for eliminating exogenous growth factor dependence of a culture comprising an avian or mammalian cell line, comprising: (a) introducing into the cell line one or more of: (i) a polynucleotide comprising a coding sequence of a growth factor ligand; (ii) a polynucleotide comprising a coding sequence of a growth factor receptor; or (iii) a polynucleotide comprising a coding sequence of an activated growth factor receptor; and (b) culturing the cell line in a cultivation infrastructure, wherein the cell line is capable of proliferating to a cell density higher than an initially seeded cell density over 72 hours of culture time without an addition of exogenous growth factors to the culture. In some embodiments, the polynucleotide sequence of (i), (ii), or (iii) are operably linked to a promoter sequence.

In some embodiments, the polynucleotide comprising a coding sequence of a growth factor ligand further comprises a coding sequence of an additional one or more growth factor ligands, wherein each of the additional growth factor ligands is selected from FGF2, IGF1, and PDGFb.

In some embodiments, the growth factor ligand is FGF2.

In some embodiments, the growth factor ligand is IGF1.

In some embodiments, the growth factor ligand is PDGFb.

In some embodiments, the coding sequence of the growth factor ligand includes a mutation for enhanced heat stability.

In some embodiments, the cell line is cultured in an absence of serum.

In some embodiments, the cell line cell density at least doubles from an initially seeded cell density.

In some embodiments, the cell line is cultured to a cell density of 1 million cells per milliliter or more.

In some embodiments, the growth factor ligand polynucleotide sequence further comprises a coding sequence of a signal peptide sequence, thereby providing a fusion protein sequence coding for a fusion protein having an increased secretion propensity relative to an endogenous growth factor.

In some embodiments,
(a) the concentration of FGF2 in the culture medium is increased by at least 2.5% as compared to cell lines not engineered to include a polynucleotide encoding FGF2;
(b) the concentration of IGF-1 in the culture medium is increased by at least 2.5% as compared to cell lines not engineered to include a polynucleotide encoding IGF1; and/or
(c) the concentration of PDGFb in the culture medium is increased by at least 2.5% as compared to cell lines not engineered to include a polynucleotide encoding PDGFb.

In some embodiments, the polynucleotide comprising a coding sequence of a growth factor receptor further comprises a coding sequence of an additional one or more growth factor receptors, wherein each additional growth factor receptor is selected from FGFR, IGFR, and PDGFR.

In some embodiments, the growth factor receptor is an FGFR selected from FGFR1, FGFR2, FGFR3, and FGFR4.

In some embodiments, the growth factor receptor is IGF1R.

In some embodiments, the growth factor receptor is PDGFR.

In some embodiments, the cell line is derived from a chicken, duck, turkey, porcine, or bovine.

In some embodiments, the cell line is derived from chicken.

In some embodiments, the cells are myoblasts, myocytes, satellite cells, side population cells, muscle derived stem cells, mesenchymal stem cells, myogenic pericytes, mesoangioblasts, fibroblasts, stem cells, or are cells otherwise having myogenic or fibroblastic capacity.

In some embodiments, the step of forming a cell line into a cell-based food product suitable for consumption, wherein the cell line and a cell culture media used to grow the grown cell are edible.

In another aspect, this disclosure features a vector comprising a polynucleotide encoding a growth factor ligand or a fragment thereof, and a growth factor receptor or a fragment thereof.

In one aspect, this disclosure features method of engineering a cell line for reduced dependence on exogenous growth factors, comprising: (a) introducing into the cell line one or more of: (i) a polynucleotide comprising a coding sequence of a growth factor ligand; (ii) a polynucleotide comprising a coding sequence of a growth factor receptor; or (iii) a polynucleotide comprising a coding sequence of an activated downstream growth factor target; and (b) culturing the cell line in a cultivation infrastructure.

In one aspect, this disclosure features method of increasing the concentration of a growth factor ligand in culture medium of cells in culture, comprising: (a) introducing into a cell line one or more of: (i) a polynucleotide comprising a coding sequence of a growth factor ligand; and (ii) a polynucleotide comprising a coding sequence of a growth factor receptor; or (iii) a polynucleotide comprising a coding sequence of an activated downstream growth factor target; and (b) culturing the cell line in a cultivation infrastructure.

In one aspect, this disclosure features method for improving anchorage independent growth in a cell line, comprising: (a) introducing into the cell line one or more of: (i) a polynucleotide comprising a coding sequence of a growth factor ligand; and (ii) a polynucleotide comprising a coding sequence of a growth factor receptor; or (iii) a polynucleotide comprising a coding sequence of an activated downstream growth factor target; and (b) culturing the cell line in a cultivation infrastructure.

In one aspect, this disclosure features method for increasing the cell density of a culture comprising a cell line, comprising: (a) introducing into the cell line one or more of: (i) a polynucleotide comprising a coding sequence of a growth factor ligand; and (ii) a polynucleotide comprising a coding sequence of a growth factor receptor; or (iii) a polynucleotide comprising a coding sequence of an activated growth factor receptor; and (b) culturing the cell line in a cultivation infrastructure.

In some embodiments, the growth factor ligand is selected from basic fibroblast growth factor (FGF2), insulin-like growth factor 1 (IGF1), and platelet-derived growth factor subunit B (PDGFb).

In some embodiments, the polynucleotide comprising a coding sequence of a growth factor ligand further comprises an additional two or more growth factor ligands, wherein each of the additional growth factor ligands is selected from FGF2, IGF1, and PDGFb.

In some embodiments, the growth factor ligands is FGF2. In some embodiments, the FGF2 comprises an amino acid sequence having at least 80% sequence identity to a sequence selected from SEQ ID NO: 1-15.

In some embodiments, the growth factor ligands is IGF1. In some embodiments, the IGF1 comprises an amino acid sequence having at least 80% sequence identity to a sequence selected from SEQ ID NO: 16-17.

In some embodiments, the growth factor ligands is PDGFb. In some embodiments, the PDGFb comprises an amino acid sequence having at least 80% sequence identity to a sequence selected from SEQ ID NO: 18-21.

In some embodiments, the polynucleotide comprising a coding sequence of a growth factor ligand further comprises a coding sequence of a signal peptide located 5′ to the coding sequence of the growth factor ligand, and wherein the signal sequence and the growth factor ligand are a fusion protein. In some embodiments, the signal sequence comprises an amino acid sequence having at least 80% sequence identity to a sequence selected from SEQ ID NO: 22-31.

In some embodiments, the polynucleotide comprising a coding sequence of a growth factor ligand further comprises a regulatory sequence operably linked to the coding sequence of the growth factor ligand and/or additional growth factor ligands.

In some embodiments, the regulatory sequence comprises a promoter selected from: an inducible promoter, a tissue specific promoter, and a constitutively active promoter.

In some embodiments, the promoter is selected from EF1alpha, PGK, CMV, RSV, and β-actin.

In some embodiments, the polynucleotide comprising a coding sequence of a growth factor ligand comprises a sequence encoding a polycistronic mRNA, wherein the polycistronic mRNA comprises the coding sequence of the growth factor ligand and the coding sequence one or more additional growth factor ligands.

In some embodiments, the polynucleotide comprising a coding sequence of a growth factor ligand comprises one or more sequences encoding a self-cleaving peptide, one or more internal ribosome entry sites (IRES), or a combination thereof.

In some embodiments, activity and/or expression of the growth factor ligand is controllable.

In some embodiments, activity and/or expression of the growth factor ligand is controlled using an inducible promoter, an inducible tag, or a degradation tag.

In some embodiments, expression of the growth factor ligands is controlled using an inducible promoter. In some embodiments, the coding sequence of the growth factor ligand is operably linked to the inducible promoter. In some embodiments, the method also includes: maintaining the engineered cell line in a culture medium comprising a molecule capable of controlling the inducible promoter, thereby enabling expression of the growth factor ligand.

In some embodiments, activity of the growth factor ligand is controlled using an inducible tag. In some embodiments, the polynucleotide comprising a coding sequence of a growth factor ligand further comprises a coding sequence of an inducible tag located 5′ or 3′ to the coding sequence of the growth factor ligand, and wherein the inducible tag and the growth factor ligand are a fusion protein. In some embodiments, the method also includes: maintaining the engineered cell line in a culture medium comprising a molecule capable of controlling the inducible tag, thereby inducing activity of the growth factor ligand/fusion protein.

In some embodiments, activity of the one or more growth factor ligands is controlled using a degradation tag. In some embodiments, the polynucleotide comprising a coding sequence of a growth factor ligand further comprises a coding sequence of a degradation tag located 5′ or 3′ to the coding sequence of the growth factor ligand, and wherein the degradation tag and the growth factor ligand are a fusion protein. In some embodiments, the method also includes: maintaining the engineered cell line in a culture medium comprising a molecule capable of controlling the degradation tag, thereby targeting the growth factor ligand/fusion protein for degradation.

In some embodiments, (a) the concentration of FGF2 in the culture medium is increased by at least 0.001 ng/mL as compared to cell lines not engineered to include a polynucleotide encoding FGF2; (b) the concentration of IGF-1 in the culture medium is increased by at least 0.001 ng/mL as compared to cell lines not engineered to include a polynucleotide encoding IGF1; and/or (c) the concentration of PDGFb in the culture medium is increased by at least 0.001 ng/mL as compared to cell lines not engineered to include a polynucleotide encoding PDGFb.

In some embodiments, (a) the concentration of FGF2 in the culture medium is increased by at least 2.5% as compared to cell lines not engineered to include a polynucleotide encoding FGF2; (b) the concentration of IGF-1 in the culture medium is increased by at least 2.5% as compared to cell lines not engineered to include a polynucleotide encoding IGF1; and/or (c) the concentration of PDGFb in the culture medium is increased by at least 2.5% as compared to cell lines not engineered to include a polynucleotide encoding PDGFb.

In some embodiments, the growth factor receptor is selected from fibroblast growth factor receptor (FGFR), insulin growth factor 1 receptor (IGF1R), and platelet-derived growth factor receptor (PDGFR).

In some embodiments, the polynucleotide comprising a coding sequence of a growth factor receptor further comprises an additional two or more growth factor receptors, wherein each additional growth factor receptor is selected from FGFR, IGFR, and PDGFR.

In some embodiments, the growth factor receptor is an FGFR selected from FGFR1, FGFR2, FGFR3, and FGFR4. In some embodiments, the FGFR comprises an amino acid sequence having at least 80% sequence identity to a sequence selected from SEQ ID NO: 32-49.

In some embodiments, the growth factor receptor is IGF1R. In some embodiments, the IGF1R comprises an amino acid sequence having at least 80% sequence identity to a sequence selected from SEQ ID NO: 50-51.

In some embodiments, the growth factor receptor is PDGFR. In some embodiments, the PDGFR comprises an amino acid sequence having at least 80% sequence identity to a sequence selected from SEQ ID NO: 52-58.

In some embodiments, the polynucleotide comprising a coding sequence of an activated downstream growth factor target comprises a growth factor receptor comprising one or more amino acid insertions, deletions, or substitutions that result in the receptor being constitutively activated.

In some embodiments, the polynucleotide comprising a coding sequence of a growth factor receptor comprises a regulatory sequence operably linked to the coding sequence of the growth factor receptor and/or the coding sequence of the additional growth factor receptors.

In some embodiments, the regulatory sequence comprises a promoter selected from: an inducible promoter, a tissue specific promoter, and a constitutively active promoter.

In some embodiments, the promoter is selected from EF1alpha, PGK, CMV, RSV, and (3-actin.

In some embodiments, the polynucleotide comprising a coding sequence of a growth factor receptor comprises a sequence encoding a polycistronic mRNA, wherein the polycistronic mRNA comprises the coding sequence of the growth factor receptor and the coding sequence of the two or more additional growth factor receptors.

In some embodiments, the polynucleotide comprising a coding sequence of a growth factor receptor comprises one or more sequences encoding a self-cleaving peptide, one or more internal ribosome entry sites (IRES), or a combination thereof.

In some embodiments, activity and/or expression of the growth factor receptors is controllable.

In some embodiments, expression of the growth factor receptor is controlled using an inducible promoter, an inducible tag, and a degradation tag.

In some embodiments, expression of the growth factor receptor is controlled using an inducible promoter. In some embodiments, the coding sequence of the growth factor receptor is operably linked to the inducible promoter. In some embodiments, the method also includes: maintaining the engineered cell line in a culture medium comprising a molecule capable of controlling the inducible promoter, thereby enabling expression of the growth factor receptor.

In some embodiments, activity of the growth factor receptor is controlled using an inducible tag. In some embodiments, the polynucleotide comprising a coding sequence of a growth factor receptor further comprises a coding sequence of an inducible tag located 5′ or 3′ to the coding sequence of the growth factor receptor, and wherein the inducible tag and the growth factor receptor are a fusion protein.

In some embodiments, the method also includes: maintaining the engineered cell line in a culture medium comprising a molecule capable of controlling the inducible tag, thereby inducing activity of the growth factor receptor/fusion protein.

In some embodiments, activity of the one or more growth factor ligands is controlled using a degradation tag. In some embodiments, the polynucleotide comprising a coding sequence of a growth factor receptor further comprises a coding sequence of a degradation tag located 5′ or 3′ to the coding sequence of the growth factor receptor, and wherein the degradation tag and the growth factor receptor are a fusion protein.

In some embodiments, the method also includes: maintaining the engineered cell line in a culture medium comprising a molecule capable of controlling the degradation tag, thereby targeting the growth factor receptor/fusion protein for degradation.

In some embodiments, the method also includes introducing a polynucleotide sequence encoding an accessory protein.

In some embodiments, the polynucleotide comprising a coding sequence of a growth factor ligand and/or the polynucleotide comprising a coding sequence of a growth factor receptor further comprises a coding sequence of an accessory protein.

In some embodiments, the accessory protein is fibroblast growth factor binding protein (FGFBP). In some embodiments, the FGFBP comprises an amino acid sequence having at least 80% sequence identity to a sequence of SEQ ID NO: 59.

In some embodiments, the accessory protein is RASV12. In some embodiments, the RASV12 comprises an amino acid sequence having at least 80% sequence identity to a sequence of SEQ ID NO: 60.

In some embodiments, the method comprises introducing into the cell: (i) a polynucleotide comprising a coding sequence of FGF2, and a polynucleotide comprising a FGFR; (ii) a polynucleotide comprising a coding sequence of IGF1, and a polynucleotide comprising an IGF1R; (iii) a polynucleotide comprising a coding sequence of PDGF, and a polynucleotide comprising a PDGFR, or (iv) a combination selected from: (i) and (ii), (ii) and (iii), (ii) and iii), and (i), (ii), and (iii).

In some embodiments, the cell line is from a livestock, poultry, game or aquatic animal species. In some embodiments, the cell line is from a chicken, duck, or turkey. In some embodiments, the cell line is from a fish. In some embodiments, the cell line is from a livestock species. In some embodiments, the livestock species is porcine or bovine. In some embodiments, the cells are from any animal species intended for human or non-human dietary consumption.

In some embodiments, the cells are myogenic cells. In some embodiments, the myogenic cells are myoblasts, myocytes, satellite cells, side population cells, muscle derived stem cells, mesenchymal stem cells, myogenic pericytes, or mesoangioblasts.

In some embodiments, the cells are non-myogenic cells.

In some embodiments, the cells are fibroblasts, stem cells, or are cells otherwise having myogenic or fibroblastic capacity.

In another aspect, this disclosure features in vitro methods for producing cell-based meat suitable for consumption, comprising: (a) introducing into a cell one or more of: (i) a polynucleotide comprising a coding sequence of a growth factor; (ii) a polynucleotide comprising a coding sequence of a growth factor receptor; or (iii) a polynucleotide comprising a coding sequence of an activated downstream growth factor target; and (b) inducing myogenic specific differentiation, wherein the differentiated cells form myocytes and multinucleated myotubes; (c) culturing the myocytes and myotubes to generate skeletal muscle fibers, thereby producing a cell-based meat suitable for consumption.

In some embodiments, the cell line is avian or mammalian. In all embodiments, the cell line is non-human. In some embodiments, the cell line is from a livestock, poultry, game or aquatic animal species. In some embodiments, the cell line is from a chicken, duck, or turkey. In some embodiments, the cell line is from a fish. In some embodiments, the cell line is from a livestock species. In some embodiments, the livestock species is porcine or bovine.

In some embodiments, the cells are from any animal species intended for human or non-human dietary consumption.

In some embodiments, the cells are myogenic cells. In some embodiments, the myogenic cells are myoblasts, myocytes, satellite cells, side population cells, muscle derived stem cells, mesenchymal stem cells, myogenic pericytes, or mesoangioblasts.

In some embodiments, the cells are non-myogenic cells.

In some embodiments, the growth factor ligand is selected from basic fibroblast growth factor (FGF2), insulin-like growth factor 1 (IGF1), and platelet-derived growth factor subunit B (PDGFb).

In some embodiments, the polynucleotide comprising a coding sequence of a growth factor ligand further comprises an additional two or more growth factor ligands, wherein each of the additional growth factor ligands is selected from FGF2, IGF1, and PDGFb.

In some embodiments, the growth factor ligands is FGF2. In some embodiments, the FGF2 comprises an amino acid sequence having at least 80% sequence identity to a sequence selected from SEQ ID NO: 1-15.

In some embodiments, the growth factor ligands is IGF1. In some embodiments, the IGF1 comprises an amino acid sequence having at least 80% sequence identity to a sequence selected from SEQ ID NO: 16-17.

In some embodiments, the growth factor ligands is PDGFb. In some embodiments, the PDGFb comprises an amino acid sequence having at least 80% sequence identity to a sequence selected from SEQ ID NO: 18-21.

In some embodiments, the polynucleotide comprising a coding sequence of a growth factor ligand further comprises a coding sequence of a signal peptide located 5′ to the coding sequence of the growth factor ligand, and wherein the signal sequence and the growth factor ligand are a fusion protein.

In some embodiments, the signal sequence comprises an amino acid sequence having at least 80% sequence identity to a sequence selected from SEQ ID NO: 22-31.

In some embodiments, the polynucleotide comprising a coding sequence of a growth factor ligand comprises a regulatory sequence operably linked to the coding sequence of the growth factor ligand and/or additional growth factor ligands.

In some embodiments, the regulatory sequence comprises a promoter selected from: an inducible promoter, a tissue specific promoter, and a constitutively active promoter.

In some embodiments, the promoter is selected from EF1alpha, PGK, CMV, RSV, and (3-actin.

In some embodiments, the polynucleotide comprising a coding sequence of a growth factor ligand comprises a sequence encoding a polycistronic mRNA, wherein the polycistronic mRNA comprises the coding sequence of the growth factor ligand and the coding sequence one or more additional growth factor ligands.

In some embodiments, the polynucleotide comprising a coding sequence of a growth factor ligand further comprises one or more sequences encoding a self-cleaving peptide, one or more internal ribosome entry sites (IRES), or a combination thereof.

In some embodiments, activity and/or expression of the growth factor ligand is controllable.

In some embodiments, activity and/or expression of the growth factor ligand is controlled using an inducible promoter, an inducible tag, or a degradation tag.

In some embodiments, expression of the growth factor ligands is controlled using an inducible promoter. In some embodiments, the coding sequence of the growth factor ligand is operably linked to the inducible promoter. In some embodiments, the method also includes: maintaining the engineered cell line in a culture medium comprising a molecule capable of controlling the inducible promoter, thereby enabling expression of the growth factor ligand.

In some embodiments, activity of the growth factor ligand is controlled using an inducible tag. In some embodiments, the polynucleotide comprising a coding sequence of a growth factor ligand further comprises a coding sequence of an inducible tag located 5′ or 3′ to the coding sequence of the growth factor ligand, and wherein the inducible tag and the growth factor ligand are a fusion protein. In some embodiments, the method also includes: maintaining the engineered cell line in a culture medium comprising a molecule capable of controlling the inducible tag, thereby inducing activity of the growth factor ligand/fusion protein.

In some embodiments, activity of the one or more growth factor ligands is controlled using a degradation tag. In some embodiments, the polynucleotide comprising a coding sequence of a growth factor ligand further comprises a coding sequence of a degradation tag located 5′ or 3′ to the coding sequence of the growth factor ligand, and wherein the degradation tag and the growth factor ligand are a fusion protein. In some embodiments, the method also includes: maintaining the engineered cell line in a culture medium comprising a molecule capable of controlling the degradation tag, thereby targeting the growth factor ligand/fusion protein for degradation.

In some embodiments, the growth factor receptor is selected from fibroblast growth factor receptor (FGFR), insulin growth factor 1 receptor (IGF1R), and platelet-derived growth factor receptor (PDGFR).

In some embodiments, the polynucleotide comprising a coding sequence of a growth factor receptor further comprises an additional two or more growth factor receptors, wherein each additional growth factor receptor is selected from FGFR, IGFR, and PDGFR.

In some embodiments, the growth factor receptor is an FGFR selected from FGFR1, FGFR2, FGFR3, and FGFR4. In some embodiments, the FGFR comprises an amino acid sequence having at least 80% sequence identity to a sequence selected from SEQ ID NO: 32-49.

In some embodiments, the growth factor receptor is IGF1R. In some embodiments, the IGF1R comprises an amino acid sequence having at least 80% sequence identity to a sequence selected from SEQ ID NO: 50-51.

In some embodiments, the growth factor receptor is PDGFR. In some embodiments, the PDGFR comprises an amino acid sequence having at least 80% sequence identity to a sequence selected from SEQ ID NO: 52-58.

In some embodiments, the polynucleotide comprising a coding sequence of an activated downstream growth factor target comprises a growth factor receptor comprising one or more amino acid insertions, deletions, or substitutions that result in the receptor being constitutively activated.

In some embodiments, the polynucleotide comprising a coding sequence of a growth factor receptor comprises a regulatory sequence operably linked to the coding sequence of the growth factor receptor and/or the coding sequence of the additional growth factor receptors.

In some embodiments, the regulatory sequence comprises a promoter selected from: an inducible promoter, a tissue specific promoter, and a constitutively active promoter.

In some embodiments, the promoter is selected from EF1alpha, PGK, CMV, RSV, and (3-actin.

In some embodiments, the polynucleotide comprising a coding sequence of a growth factor receptor comprises a sequence encoding a polycistronic mRNA, wherein the polycistronic mRNA comprises the coding sequence of the growth factor receptor and the coding sequence of the two or more additional growth factor receptors.

In some embodiments, the polynucleotide comprising a coding sequence of a growth factor receptor comprises one or more sequences encoding a self-cleaving peptide, one or more internal ribosome entry sites (IRES), or both.

In some embodiments, activity and/or expression of the growth factor receptors is controllable.

In some embodiments, expression of the growth factor receptor is controlled using an inducible promoter, an inducible tag, and a degradation tag.

In some embodiments, expression of the growth factor receptor is controlled using an inducible promoter. In some embodiments, the coding sequence of the growth factor receptor is operably linked to the inducible promoter. In some embodiments, the method also includes: maintaining the engineered cell line in a culture medium comprising a molecule capable of controlling the inducible promoter, thereby enabling expression of the growth factor receptor.

In some embodiments, activity of the growth factor receptor is controlled using an inducible tag. In some embodiments, the polynucleotide comprising a coding sequence of a growth factor receptor further comprises a coding sequence of an inducible tag located 5′ or 3′ to the coding sequence of the growth factor receptor, and wherein the inducible tag and the growth factor receptor are a fusion protein. In some embodiments, the method also includes: maintaining the engineered cell line in a culture medium comprising a molecule capable of controlling the inducible tag, thereby inducing activity of the growth factor receptor/fusion protein.

In some embodiments, activity of the one or more growth factor ligands is controlled using a degradation tag. In some embodiments, the polynucleotide comprising a coding sequence of a growth factor receptor further comprises a coding sequence of a degradation tag located 5′ or 3′ to the coding sequence of the growth factor receptor, and wherein the degradation tag and the growth factor receptor are a fusion protein. In some embodiments, the method also includes: maintaining the engineered cell line in a culture medium comprising a molecule capable of controlling the degradation tag, thereby targeting the growth factor receptor/fusion protein for degradation.

In some embodiments, the method also includes introducing a polynucleotide sequence encoding an accessory protein.

In some embodiments, the polynucleotide comprising a coding sequence of a growth factor ligand, the polynucleotide comprising a coding sequence of a growth factor receptor, or both, further comprises a coding sequence of an accessory protein.

In some embodiments, the accessory protein is fibroblast growth factor binding protein (FGFBP). In some embodiments, the FGFBP comprises an amino acid sequence having at least 80% sequence identity to a sequence of SEQ ID NO: 59.

In some embodiments, the accessory protein is RASV12. In some embodiments, the RASV12 comprises an amino acid sequence having at least 80% sequence identity to a sequence of SEQ ID NO: 60.

In some embodiments, the method comprises introducing into the cell: (i) a polynucleotide comprising a coding sequence of FGF2, and a polynucleotide comprising a coding sequence of FGFR; (ii) a polynucleotide comprising a coding sequence of IGF1, and a polynucleotide comprising a coding sequence of IGF1R; (iii) a polynucleotide comprising a coding sequence of PDGF, and a polynucleotide comprising a coding sequence of PDGFR, or (iv) a combination selected from: (i) and (ii), (ii) and (iii), (ii) and iii), and (i), (ii), and (iii).

In another aspect, this disclosure features a myogenic cell suitable for consumption comprising cells having increased expression of FGF2, IGF1, PDGFb, FGFR, IGF1R, or PDGFR, or a combination thereof.

In another aspect, this disclosure features a population of cells suitable for consumption comprising cells having increased expression of FGF2, IGF1, PDGFb, FGFR, IGF1R, or PDGFR, or a combination thereof.

In another aspect, this disclosure features a vector comprising a polynucleotide encoding a fusion protein comprising a coding sequence of a signaling sequence and a coding sequence of a growth factor ligand, and optionally a coding sequence of an inducible tag or a degradation tag.

In another aspect, this disclosure features a cell comprising a polynucleotide encoding a fusion protein comprising a coding sequence of a signaling sequence and a coding sequence of a growth factor ligand, and optionally a coding sequence of an inducible tag or a degradation tag.

In another aspect, this disclosure features a vector comprising a polynucleotide comprising a first coding sequence of a growth factor ligand, and a second coding sequence of growth factor receptor.

In another aspect, this disclosure features a cell comprising a polynucleotide comprising a coding sequence of a growth factor ligand or a fragment thereof, and a polynucleotide comprising a coding sequence of a growth factor receptor or a fragment thereof.

In another aspect, this disclosure features cell-based meat suitable for consumption produced using the any of the methods described herein.

### 5. BRIEF DESCRIPTION OF THE DRAWINGS

These and other features, aspects, and advantages of the present invention will become better understood with regard to the following description, and accompanying drawings, where:

These and other features, aspects, and advantages of the present invention will become better understood with regard to the following description, and accompanying drawings, where:

FIG. 1 shows illustration of three approaches to generate growth factor independent cell lines. Specific growth factors required for cell proliferation and/or tissue formation phenotypes are identified by conducting a growth factor screen and/or analysis for upregulated growth factor receptors. Growth factor-independent cell lines are generated with the following approaches: 1. Genetically engineer cells to overexpress native growth factor or growth factor variants with higher thermostability (heat stable), increased half-life, and/or enhanced secretions. 2. Genetically engineer cells to overexpress native downstream growth factor targets such as growth factor receptors or growth factor receptor variants with constitutively active signaling. 3. Genetically engineer cells to overexpression both growth factor and activated downstream target.

FIG. 2A shows sequence alignment of amino acid sequences for FGF2 from human, chicken, bovine, and salmon. Chicken FGF2 amino acid sequence has 91.6%, 92.9%, and 76.1% identity with human, bovine, and salmon FGF2 sequences, respectively.

FIG. 2B shows a sequence alignment of amino acid sequences for IGF1 (truncated) from chicken, bovine (cow), pig, and human.

FIG. 2C shows a sequence alignment of amino acid sequences for PDGFb from chicken, bovine, and human. Chicken PDGFb sequence has 53.5% and 53.1% identity with bovine and human sequences, respectively. Bovine and human PDGFb sequences share 90.5% identity.

FIG. 3A shows a schematic of a vector used for promoter selection experiments. Gene of interest is expressed from a mouse PGK promoter.

FIG. 3B shows a schematic of a vector used for promoter selection experiments. Gene of interest is expressed from a human EF1alpha promoter.

FIG. 4 is a bar graph showing the fold change (RLU) in expression for the promoter analysis performed in Example 1. Chicken cells were transiently transfected (n=3) with vectors expressing nano luciferase (NLucP) under control of a mouse PGK promoter or human EF1a promoter. After 24 hrs, luciferase expression was measured and depicted as relative light units (RLU). p<0.001(**).

FIG. 5 is a bar graph showing FGF2 concentration in the supernatant after 72 hours in culture (serum-free) for cell lines expressing: mPGK-BM40-FGF2-WT and hEF1a-BM40-FGF2-WT. Abbreviations: PGK—PKG promoter, BM40—signal peptide; FGF2—growth factor ligand; and WT—wild type FGF2.

FIG. 6 is bar graph showing the FGF2 concentration in the supernatant in culture medium for the various transduction conditions. X-axis nomenclature: “signal peptide-growth factor ligand” (e.g., for “GLuc-FGF2” GLuc is signal peptide fused to FGF2).

FIG. 7 is a bar graph showing cell proliferation as fold change for each of the various transduction conditions noted on the x-axis. Top dotted line indicates fold change for a serum containing positive control (i.e., cells transduced with empty vector (EV) and cultured in serum). Bottom dotted line indicates fold change for a serum containing positive control (i.e., cells transduced with empty vector) and grown in media include 100 ng/mL FGF2. X-axis nomenclature: “signal peptide-growth factor ligand” (e.g., for “GLuc-FGF2” GLuc is signal peptide fused to FGF2).

FIG. 8 shows representative images of control (FC550A) and FGF2-WT cells plated in serum free media not supplemented with FGF2. Images were taken 12 hours after initial seed at 4× magnification.

FIG. 9 shows representative images of control (FC550A) and FGF2-WT cells plated in serum free media not supplemented with FGF2. Images were taken 48 hours after initial seed at 4× magnification. Arrow indicates a secondary cell population that was not attached to the plate.

FIG. 10 is a bar graph showing Viable Cell Densities (VCD) for each of the various transduction conditions indicated on the x-axis. Top dotted line indices the VCD for a positive control (i.e., cells transduced with empty vector (EV) and cultured in media supplemented with 100 ng/mL FGF2). Bottom dotted line indicates the VCD for a negative control (i.e., cells transduced with empty vector (EV) and cultured in media not supplemented with FGF2). X-axis nomenclature: “signal peptide-growth factor ligand” (e.g., for “GLuc-FGF2” GLuc is signal peptide fused to FGF2).

FIG. 11 is a bar graph showing Viable Cell Densities (VCD) for each of the various transduction conditions indicated on the x-axis. Dotted line indicates the seeding density of 0.4×10E6 cells/mL. X-axis nomenclature: “signal peptide-growth factor ligand” (e.g., for “GLuc-FGF2” GLuc is signal peptide fused to FGF2).

FIG. 12 is a bar graph showing Viable Cell Densities (VCD) for each of the cell lines indicated on the x-axis: chicken FGF2, bovine FGF2, salmon FGF2, heat stable variants of FGF2 (e.g., FGF2-Q68I-N114G and FGF2-Q68I-C99S-N114G), FGF2 having a secretion tags (e.g., ggIL2-FGF2 and hIL2-col-ggFGF2(xMet)), FGF2 variants that preserve amino acid for non-canonical secretion (FGF2-C77-Y81), and FGF2-STAB variants (STAB-C77Y81, c96-STAB-Gen2-ggFGF2, STAB-Gen3-ggFGF2, and c96-STAB-Gen3-ggFGF2). VCD data was collected on day 2 of passage 4.

FIG. 13 is a bar graph showing percent (%) Packed Cell Volumes for each of the cell lines indicated on the x-axis: chicken FGF2, bovine FGF2, salmon FGF2, heat stable variants of FGF2 (e.g., FGF2-Q68I-N114G and FGF2-Q68I-C99S-N114G), FGF2 having a secretion tags (e.g., ggIL2-FGF2 and hIL2-col-ggFGF2(xMet)), and FGF2 variants that preserve amino acid for non-canonical secretion (FGF2-C77-Y81). Percent packed cell volumes were assessed on day 2 of passage 5.

FIG. 14 is a bar graph of an ELISA showing FGF2 concentration in (pg/mL) in supernatants taken from the cultures indicated on the x-axis: chicken FGF2, bovine FGF2, salmon FGF2, heat stable variants of FGF2 (e.g., FGF2-Q68I-N114G and FGF2-Q68I-C99S-N114G), FGF2 having a secretion tags (e.g., ggIL2-FGF2 and hIL2-col-ggFGF2(xMet)), and FGF2 variants that preserve amino acid for non-canonical secretion (FGF2-C77-Y81).

FIG. 15 is a bar graph of an ELISA showing Viable Cell Density (VCD) for each of the cell lines indicated on the x-axis. FGF1 receptors included: FGFR1C, FGFR1C-N546K, FGFR1C-V561M, and myrist-FGFR1C-K656E. FGF2 receptors included: FGFR2, FGFR2-N550K, FGFR2-K660E, and FGFR2-3xMuts. FGF3 receptors included: FGFR3, FGFR3-N540K, FGFR3-K560E, FGFR3-N540K-K560E, and myrist-FGF3R-K560E. FGF4 receptors included: FGF4R, FGF4R-Y367C, and FGF4R-Y367C-K654E.

FIG. 16 is a bar graph showing percent (%) Packed Cell Volumes for each of the cell lines indicated on the x-axis. FGF1 receptors included: FGFR1C, FGFR1C-N546K, FGFR1C-V561M, and myrist-FGFR1C-K656E. FGF2 receptors included: FGFR2, FGFR2-N550K, FGFR2-K660E, and FGFR2-3xMuts. FGF3 receptors included: FGFR3, FGFR3-N540K, FGFR3-K560E, FGFR3-N540K-K560E, and myrist-FGF3R-K560E. FGF4 receptors included: FGF4R, FGF4R-Y367C, and FGF4R-Y367C-K654E.

FIG. 17 is a bar graph of an ELISA showing FGF2 concentration in (pg/mL) in supernatants taken from the cultures indicated on the x-axis. FGF1 receptors included: FGFR1C, FGFR1C-N546K, FGFR1C-V561M, and myrist-FGFR1C-K656E. FGF2 receptors included: FGFR2, FGFR2-N550K, FGFR2-K660E, and FGFR2-3xMuts. FGF3 receptors included: FGFR3, FGFR3-N540K, FGFR3-K560E, FGFR3-N540K-K560E, and myrist-FGF3R-K560E. FGF4 receptors included: FGF4R, FGF4R-Y367C, and FGF4R-Y367C-K654E.

FIG. 18 is a bar graph showing viable cell density data for chicken cells engineered to express IGF1 (or variant) or IGF1 receptor (or variant).

FIG. 19 is a bar graph showing packed cell volume data for chicken cells engineered to express IGF1 (or variant) or IGF1 receptor (or variant).

FIGS. 20A-20B are line graphs showing growth curves (Viable Cell Densities (VCD)) for controls (Naïve and empty vector (EV)) and IGF1 engineered cells. Cells are grown in media supplemented with either 0 μg/L IGF1 or 10 μg/L IGF1. FIG. 20A shows VCD data for cells engineered to express low levels of IGF1 (IGF1^{Low}) and controls with measurements taken every 24 hours and cells passaged on a 2-day cadence (e.g., about every 48 hours). FIG. 20B shows VCD data for cells engineered to express high levels of IGF1 (IGF1^{High}) and controls with measurements taken every 24 hours and cells passaged on a 2-day cadence (e.g., about every 48 hours).

FIGS. 21A-21B are bar graphs showing data from an IGF1 ELISA for cells engineered to express IGF1^{low} or IGF1^{high}. FIG. 21A shows IGF1 ELISA data for cells engineered to express IGF1^{low}. FIG. 21B shows IGF1 ELISA data for cells engineered to express IGF1^{high}.

FIG. 22 are bar graphs showing doubling time (hours) data for cells engineered to express IGF1^{low}.

FIGS. 23A-23B are bar graphs showing Viable Cell Densities (VCD) for controls (Naïve and empty vector (EV)) and IGF1 engineered cells. Cells are grown in media supplemented with either 0 μg/L IGF1 or 10 μg/L IGF1. Cultures are analyzed at day 2 after the third passage. FIG. 23A shows VCD data for cells engineered to express low levels of IGF1 (IGF1^{Low}) and controls. FIG. 23B shows VCD data for cells engineered to express high levels of IGF1 (IGF1^{High}) and controls.

FIGS. 23C-23D are bar graphs showing Packed Cell Volumes (PCV) for controls (Naïve and empty vector (EV)) and IGF1 engineered cells. Cells are grown in media supplement with either 0 μg/L IGF1 or 10 μg/L IGF1. Cultures are analyzed at day 2 after the third passage. FIG. 23C shows PCV data for cells engineered to express low levels of IGF1 (IGF1^{Low}) and controls. FIG. 23D shows PCV data for cells engineered to express high levels of IGF1 (IGF1^{High}) and controls.

FIG. 24 shows doubling time (hours) for chicken for chicken cells engineered to express IGF1 (IGF1^{low}) grown in media supplement without (“−”) and with (“+”) IGF1.

FIG. 25 shows packed cell volume estimated yield (g/L) for chicken cells engineered to express IGF1 (IGF1^{low}) grown in media supplement without (“−”) and with (“+”) IGF1.

FIG. 26 shows viable cell density data for chicken cells engineered to express IGF1 and a FGF2 variant (chicken (Chicken FGF2), bovine (Bovine FGF2), salmon (Salmon FGF2), heat stable variants (FGF2-Q68I-N114G, FGF2-Q68I-C99S-N114G), secretion tagged variants (ggIL2-FGF2, hIL2-col-ggFGF2(xMet)) or variant to preserve amino acid for non-canonical secretion (FGF2-C77Y81)). Controls grown with or without growth factors IGF1 (10 ug/L) and FGF2 (100 ug/L) consisted of dual empty vector (EVmRuby and EVeBFP2) that were used to construct IGF1 and FGF2 variants, respectively.

FIG. 27 shows that viable cell density data for chicken cells engineered to express IGF1 and a FGF receptor (FGFR1c-N546K, FGFR1c-V561M, myrist-FGFR1c-K656E, FGFR3-N540K, FGFR3-K650E, FGFR3-N540K-K650E, or myrist-FGFR3-K650E). Controls grown with or without growth factors IGF1 (10 ug/L) and FGF2 (100 ug/L) consisted of dual empty vector (EVmRuby and EVeBFP2) that were used to construct IGF1 and FGF receptor variants, respectively.

FIG. 28 shows that viable cell density data for chicken cells engineered to expressed FGFR2. Naïve chicken cells were used a controls.

FIGS. 29A-29C show viable cell density (VCD) data over 200 hours in culture. FIG. 29A shows VCD data for chicken cells engineered to express FGF2 from different species (chicken, bovine, and salmon). FIG. 29B shows VCD data for chicken cells engineered to express FGF2 variants that have amino acid substitutions that increase heat stability. FIG. 29C shows VCD data for chicken cells engineered to express FGF2 designed with secretion signals or variants to preserve non-canonical secretion.

FIG. 30 show viable cell density (VCD) data summarizing FIGS. 29A-29C.

FIGS. 31A-31C show viable cell density (VCD) data over 200 hours in culture for chicken cells engineered to express the indicated FGF receptor variants. FIG. 31A shows VCD data for chicken cells engineered to express variants of FGFR1. FIG. 31B shows VCD data for chicken cells engineered to express FGFR2 variants. FIG. 31C shows VCD data for chicken cells engineered to express FGFR3 variants.

FIG. 32 shows viable cell density (VCD) data) for chicken cells engineered to express the indicated FGF receptor variants. One way ANOVA performed compared to empty vector control in growth factor containing media, Not significant (n.s.), p<0.05 (*), p<0.001(**), p<0.0001(***)p<0.0001 (****).

FIG. 33 shows Wet Mass per Area (pg/cm²) for engineered cells and controls cultured in roller bottles over 14 day period in serum free media. Controls include Naive and empty vector (EV) cells grown with/without 50 ug/L PDGF (N=4). Chicken cells were engineered to express PDGFRa-D842V, PDGF(WT) or PDGF-211*.

FIGS. 34A-34D show an overview diagram of growing and processing different types of cells in accordance with one or more embodiments of the present disclosure. FIG. 34A shows tissue collection, processing, culturing, and cryopreserving. FIG. 34B shows immortalizing, culturing until confluency, suspension culturing, cryopreserving, and expanding in larger culture vessels. FIG. 34C shows a bioreactor system. FIG. 34D shows a pressure apparatus that compresses cell masses.

### 6. DETAILED DESCRIPTION

### 6.1. Definitions

Unless defined otherwise, all technical and scientific terms used herein have the same meaning as is commonly understood by one of ordinary skill in the art to which this disclosure belongs. In the event that there is a plurality of definitions for terms cited herein, those in this section prevail unless otherwise stated.

Throughout this disclosure, the term “a” or “an” entity refers to one or more of that entity. As such, the terms “a” (or “an”), “one or more,” and “at least one” can be used interchangeably herein. Furthermore, “and/or” as used in a phase such as “A and/or B” herein is intended to include “A and B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the term “accessory protein” refers to a protein that modulates the actions or downstream signaling of a growth factor ligand or a growth factor receptor. Accessory proteins can modulate the actions of growth factor ligand and/or a growth factor receptor either directly (e.g., binding/interacting directly) or indirectly.

As used herein, the terms “comprising” and “including” or grammatical variants thereof are to be taken as specifying the stated features, integers, steps, or components but do not preclude the addition of one or more additional features, integers, steps, components, or groups thereof. This term encompasses the terms “consisting of” and “consisting essentially of”.

As used herein, the terms “cell” and “cell line” are sometimes used interchangeably. As used herein, the term “cell” can refer to one or more cells originating from a cell line. As used herein, the term “cell line” can refer to a population of cells.

As used herein, the terms “cell surface” or “surface of the cell” when referring to a receptor refers to the presence of the receptor on the surface of the cell.

As used herein, the term “cultivation infrastructure” refers to the environment in which the cells, cell lines, myocytes, multinucleated myotubes, or skeletal muscle fibers are cultured.

As used herein, the term “differentiation capacity” refers to a cell's ability to differentiate to a particular cell lineage, stem cell, progenitor cell, or terminally differentiated cell.

As used herein, the term “exogenous,” when referring to growth factors, refers to a growth factor derived from a source external to the culture and added (supplemented) to the culture medium.

As used herein, the term “fragment” or “portion” when referring to a protein or a polynucleotide refers to a protein that comprises a domain, portion, or fragment of a parent or reference protein or polypeptide. The term “portion” can be used interchangeably with the term “functional portion.” The term “fragment” can be used interchangeably with the term “functional fragment.” The terms “functional portion” or “functional fragment” refers to components that retain at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent protein or polypeptide, or provides a biological benefit. A “functional portion” or “functional fragment” of a protein or polypeptide has “similar binding” or “similar activity” when the functional portion or fragment displays no more than a 50% reduction in performance in a selected assay as compared to the parent or reference protein or polypeptide (preferably no more than 20% or 10%, or no more than a log difference as compared to the parent or reference with regard to affinity).

As used herein, the terms “growth factor ligand” refers to a secreted biologically active molecule that can affect the growth of cells, promote or inhibit mitosis, or affect cellular differentiation.

As used herein, the term “immortalized cell” refers to cells that are passaged or modified to proliferate indefinitely and evade normal cellular senescence.

As used herein, the term “myoblast” refers to mononucleated muscle cells. They are embryonic precursors of myocytes, also called muscle cells. Although myoblasts may be classified as skeletal muscle myoblasts, smooth muscle myoblasts, and cardiac muscle myoblasts depending on the type of muscle cell that they will differentiate into, in this specification the term myoblasts refer to skeletal muscle myoblasts.

As used herein, the term “myotube” refers to elongated structures, the result of differentiated myoblast. Upon differentiation, myoblasts fuse into one or more nucleated myotubes and express skeletal muscle markers.

As used herein the term “passaged cell” refers to the number of times the cells in the culture have been subcultured. This may occur without consideration of the inoculation densities or recoveries involved.

As used herein, the term “population doubling level (PDL)” refers to the total number of times the cells in the population have doubled since their primary isolation in vitro. Mathematically this is described as n=3.32 (log UCY−log 1)+X, where n=the final PDL number at end of a given subculture, UCY=the cell yield at that point, 1=the cell number used as inoculum to begin that subculture, and X=the doubling level of the inoculum used to initiate the subculture being quantitated.

As used herein, the term “substantially free of” or “substantially free from” means the amount (e.g., absolute number within a population or concentration/percentage within a population) of a cell or cell type is below a value where the cell or cell type, or any cell derived therefrom, could contribute to the population. For example, a population substantially free of a cell means that upon differentiation of the population the cell does not sustain progeny in the differentiated population. When referring to culture reagents, “substantially free of” or “substantially free from” refer to the amount (e.g., concentration) of the reagent that is below a value where the cell culture reagent does not have a biological effect on the culture (i.e., the reagent is not capable of producing a biological effect at such low concentrations).

As used herein, the term “transdifferentiation” refers to the conversion of a cell type present in one tissue or organ into a cell type from another tissue or organ without going through a pluripotent cell state. Transdifferentiation between some cell types can occur naturally. In other cases, transdifferentiation can be induced using exogenous factors including small molecules, growth factors, and/or genetic engineering.

As used herein, the terms “transformed,” “transduced,” and “transfected” are used interchangeably unless otherwise noted. Each term refers to introduction of a nucleic acid sequence or polypeptide into a cell (e.g., an immortalized cell).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit, unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

### 6.2. Growth Factor Ligands

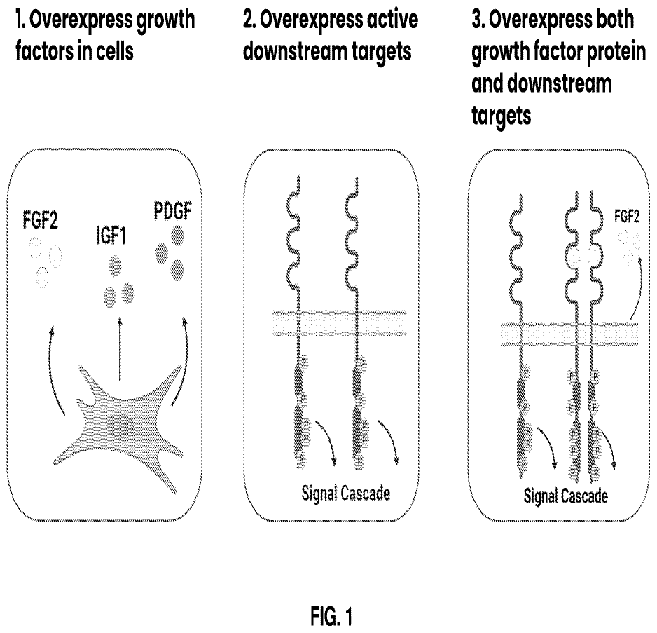

Provided herein are methods for introducing into a cell a polynucleotide comprising a coding sequence of a growth factor ligand (see FIG. 1). In some embodiments, introducing the polynucleotide comprising the coding sequence of the growth factor ligand into the cells results in the cells having reduced reliance on exogenous growth factors. In such cases, the cells are engineered to overexpress the coding sequence of the growth factor ligand. In some embodiments, the cells are genetically engineered to have stable integration of the one or more copies of a coding sequence for a growth factor ligand. In some embodiments, the cells overexpress the coding sequence of the growth factor ligand at levels sufficient to increase production and/or secretion of the growth factor ligands into the cell culture medium. In some embodiments, the growth factor ligand is selected from basic fibroblast growth factor (FGF2), insulin-like growth factor 1 (IGF1), insulin-like growth factor 2 (IGF2), and platelet-derived growth factor (PDGF). The FGF2, IGF1, IGF2, PDGF is selected from any metazoan species.

In some embodiments, the methods provided herein include introducing a polynucleotide comprising a coding sequence of FGF2, IGF1, IGF2, PDGF, or a combination thereof. For example, the polynucleotide comprises a coding sequence of FGF2, FGF2 and IGF1, FGF2 and PDGF, IGF1 and PDGF, or FGF2, IGF1 and PDGF.

### 6.2.1. Fibroblast Growth Factor 2

In some embodiments, the methods provided herein include introducing into a cell a polynucleotide comprising a coding sequence of fibroblast growth factor 2 (FGF2) or a fragment thereof. As used herein, “FGF2” refers to the fibroblast growth factor 2 (Fgf2) gene or FGF2 protein, which is a member of the fibroblast growth factor (FGF) family. FGF family members bind heparin and possess broad mitogenic and angiogenic activities. FGF2: acts as a ligand for FGFR1, FGFR2, FGFR3 and FGFR4; acts as an integrin ligand which is required for FGF2 signaling; binds to integrin ITGAV:ITGB3; plays an important role in the regulation of cell survival, cell division, cell differentiation and cell migration; functions as a potent mitogen in vitro; induces angiogenesis; and mediates phosphorylation of ERK1/2.

In some embodiments, the cells are modified to overexpress the coding sequence of an FGF2 protein. In some embodiments, the cells are genetically modified and carry stable integrations of one or more copies of an FGF2 coding sequence. In some embodiments, the cells overexpress the coding sequence of FGF2 protein at levels sufficient to increase production and/or secretion of FGF2 into the cell culture medium.

In some embodiments, the FGF2 coding sequence is selected from any metazoan species. In some embodiments, the FGF2 coding sequence is from any animal, such as vertebrate and invertebrate animal species. In some embodiments, the FGF2 coding sequence is from any vertebrate animal species such as mammals, reptiles, birds, amphibians, and the like. In some embodiments, the FGF2 coding sequence is from any mammalian species such as a human, murine, bovine, porcine, poultry, and the like. In some embodiments, the coding sequence of the FGF2 protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix, Copra aegagrus hircus, or Homarus americanus.

In some embodiments, increasing expression of FGF2 may be achieved using different approaches. In some embodiments, the expression is inducible. In some embodiments, the method comprises expressing polynucleotides comprising the coding sequence of FGF2. In some embodiments, the polynucleotides are ectopically expressed from constructs that are introduced into the cells, for example expressed from a plasmid, or other expression vector. In some embodiments, the constructs are integrated into the cell's genome, and the expression is driven in that manner (e.g., PhiC31 Integration Systems). In some embodiments, the expression of the FGF2 gene involves electroporating a DNA, delivering a DNA complexed with a transfection vehicle, using a viral vector (e.g. retrovirus, lentivirus, adenovirus, adeno-associated virus), and the like, or combinations thereof. In some embodiments, the expression is constitutive. In some embodiments, the expression is conditional (e.g. inducible).

In the methods described herein, a polynucleotide comprising a coding sequence of FGF2 may encode any homolog of FGF2, including FGF2 paralogs, such as FGF1, FGF3, FGF5, FGF9, and FGF16, or any other FGF2 paralogs, or an FGF2 protein translated from any splice variants of an FGF2 gene, or may comprise any mutations in the FGF2 gene sequence including, but not limited to nucleotide deletions, truncations, fusions, or substitutions. Mutations may be synthetic or naturally occurring.

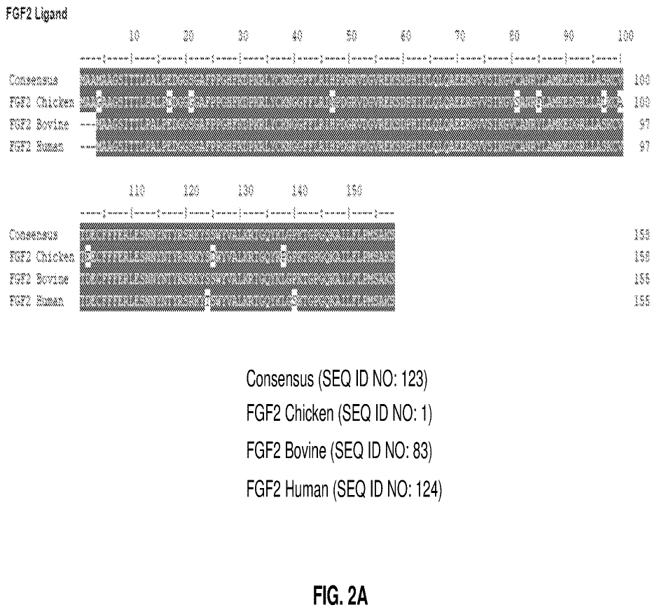

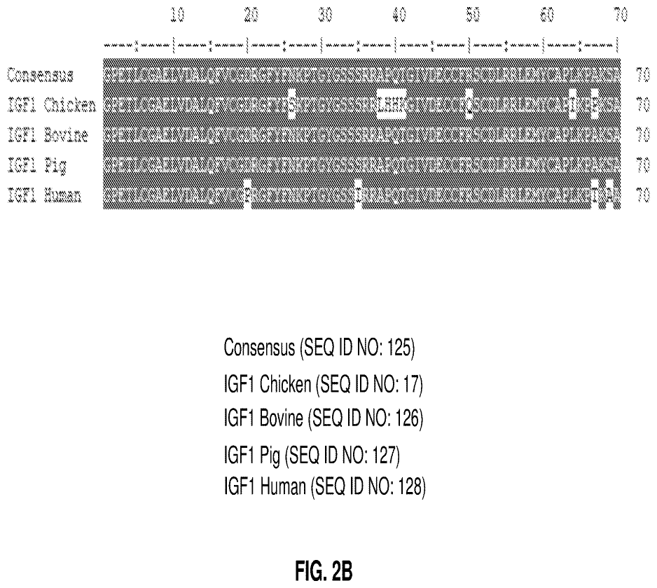

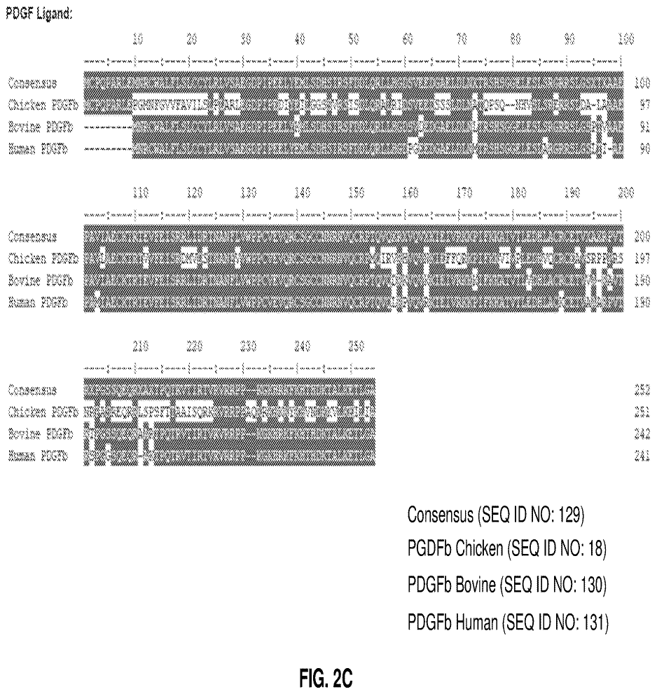

In some embodiments, FGF2 refers to the Fgf2 gene or FGF2 protein, or fragment or variant thereof (e.g., a FGF2 protein having one or more (e.g., one, two, three, four, five, six, seven, eight, nine, ten, or more amino acid substitutions, deletions or insertions as compared to a wild type FGF2 protein)). FIG. 2A shows a sequence alignment for FGF2 from chicken, bovine and human. In some embodiments, FGF2 refers to a sequence listed in FIG. 2A.

In some embodiments, a FGF2 protein comprises an amino acid sequence having at least 80% (e.g., at least 85%, 90%, 95%, 96%, 97%, 98%, or 99%) sequence identity to a sequence selected from SEQ ID NOs: 1-15. In some embodiments, the FGF protein sequence comprises an amino acid sequence selected from SEQ ID NOs: 1-15.

In some embodiments, the FGF2 protein is a wild type (WT) chicken FGF2 (SEQ ID NO: 1). In some embodiments, the FGF2 is a stabilized version of wild type chicken FGF2 (SEQ ID NO: 2). In such embodiments, the FGF2 comprises one or more amino acid substitutions engineered to impart increased thermostability on the FGF2 protein (i.e., increase half-life in the culture medium). In some embodiments, a thermostable FGF2 is referred to as a STAB FGF2. In some embodiments, the one or more amino acid substitutions are selected from R31L, V52T, E54D, H59F, L92Y, S94I, C96N, S109E, T121P as compared to SEQ ID NO: 1.

In some embodiments, introducing the polynucleotide comprising the coding sequence of the FGF2 protein alone is not sufficient to confer reduced reliance on exogenous growth factors. In such embodiments, one or more additional growth factor ligands can be introduced into the cell line. For example, a polynucleotide comprising the coding sequence of a IGF1 protein or a PDGF protein can be introduced into the cell line to help confer reduced reliance on exogenous growth factors. In other embodiments, a polynucleotide comprising a coding sequence of a growth factor receptor can be introduced into the cell line to help confer reduced reliance on exogenous growth factors. In such embodiments, the growth factor receptor can be selected from a FGF2R, an IGF-1R, and a PDFGR. For example, in order to confer reduced reliance on exogenous growth factors on the cell line, a polynucleotide comprising a coding sequence of a growth factor ligand (e.g., FGF2) or a fragment thereof, and a polynucleotide comprising a coding sequence of a growth factor receptor (e.g., FGFR) are introduced into the cell line.

### 6.2.2. Insulin Growth Factor 1

In some embodiments, the methods provided herein include introducing into a cell a polynucleotide comprising a coding sequence of Insulin-like growth factor 1 (IGF1) or a fragment thereof. As used herein, “IGF1” refers to the insulin-like growth factor 1 (Igf1) gene or IGF1 protein that is gene similar to insulin in function and is a member of a family of proteins involved in mediating growth and development. Without wishing to be bound by theory, IGF1 acts as a ligand for insulin-like growth factor 1 receptor (IGF1R). IGF-1 binds to the alpha subunit of IGF1R, leading to the activation of the intrinsic tyrosine kinase activity which autophosphorylates tyrosine residues in the beta subunit thus initiating a cascade of down-stream signaling events leading to activation of the PI3K-AKT/PKB and the Ras-MAPK pathways. IGF1 can bind to integrins ITGAV:ITGB3 and ITGA6:ITGB4. Its binding to integrins and subsequent ternary complex formation with integrins and IGFR1 are important for IGF1 signaling. IGF1 induces the phosphorylation and activation of IGFR1, MAPK3/ERK1, MAPK1/ERK2 and AKT1.

In some embodiments, the cells are modified to overexpress the coding sequence of an IGF1 protein. In some embodiments, the cells are genetically modified and carry stable integrations of one or more copies of an IGF1 coding sequence. In some embodiments, the cells overexpress the coding sequence of IGF1 protein at levels sufficient to increase production and/or secretion of IGF1 into the cell culture medium.

In some embodiments, the IGF1 coding sequence is selected from any metazoan species. In some embodiments, the IGF1 coding sequence is from any animal, such as vertebrate and invertebrate animal species. In some embodiments, the IGF1 coding sequence is from any vertebrate animal species such as mammals, reptiles, birds, amphibians, and the like. In some embodiments, the IGF1 coding sequence is from any mammalian species such as a human, murine, bovine, porcine, poultry, and the like. In some embodiments, the coding sequence of the IGF1 protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus.

In some embodiments, increasing expression of IGF1 may be achieved using different approaches. In some embodiments, the expression is inducible. In some embodiments, the method comprises expressing polynucleotides comprising the coding sequence of IGF1. In some embodiments, the polynucleotides are ectopically expressed from constructs that are introduced into the cells, for example expressed from a plasmid, or other expression vector. In some embodiments, the constructs are integrated into the cell's genome, and the expression is driven in that manner (e.g., PhiC31 Integration Systems). In some embodiments, the expression of the IGF1 gene involves electroporating a DNA, delivering a DNA complexed with a transfection vehicle, using a viral vector (e.g. retrovirus, lentivirus, adenovirus, adeno-associated virus), and the like, or combinations thereof. In some embodiments, the expression is constitutive. In some embodiments, the expression is conditional (e.g. inducible).

In the methods described herein, a polynucleotide comprising a coding sequence of IGF1 may encode any homolog of IGF1, including IGF1 paralogs, such as IGF2, INS, and INS-IGF2, or any other IGF1 paralogs, or an IGF1 protein translated from any splice variants of an IGF1 gene, or may comprise any mutations in the IGF1 gene sequence including, but not limited to nucleotide deletions, truncations, fusions, or substitutions. Mutations may be synthetic or naturally occurring.

In some embodiments, IGF1 refers to the Igf1 gene or IGF1 protein, or fragment or variant thereof (e.g., a IGF1 protein having one or more (e.g., one, two, three, four, five, six, seven, eight, nine, ten, or more amino acid substitutions, deletions or insertions as compared to a wild type IGF1 protein)). FIG. 2B shows a sequence alignment for IGF1 from chicken, bovine, pig and human. In some embodiments, IGF1 refers to a sequence listed in FIG. 2B.

In some embodiments, an IGF1 protein comprises an amino acid sequence having at least 80% (e.g., at least 85%, 90%, 95%, 96%, 97%, 98%, or 99%) sequence identity to a sequence selected from SEQ ID NO: 16 or 17. In some embodiments, the FGF protein sequence comprises an amino acid sequence selected from SEQ ID NO: 16 or 17.

In some embodiments, introducing the polynucleotide comprising the coding sequence of the IGF-1 protein alone is not sufficient to confer reduced reliance on exogenous growth factors. In such embodiments, one or more additional growth factor ligands can be introduced into the cell line. For example, a polynucleotide comprising the coding sequence of a FGF2 protein or a PDGF protein can be introduced into the cell line to help confer reduced reliance on exogenous growth factors. In other embodiments, a polynucleotide (e.g., a second polynucleotide) comprising a coding sequence of a growth factor receptor can be introduced into the cell line to help confer reduced reliance on exogenous growth factors. In such embodiments, the growth factor receptor can be selected from a FGF2R, a IGF1R, and a PDFGR. For example, in order to confer reduced reliance on exogenous growth factors on the cell line, a polynucleotide comprising a coding sequence of a growth factor ligand (e.g., IGF1) or a fragment thereof, and a polynucleotide comprising a coding sequence of a growth factor receptor (e.g., IGF-1R) are introduced into the cell line.

### 6.2.3. Platelet Derived Growth Factor

In some embodiments, the methods provided herein include introducing into a cell a polynucleotide comprising a coding sequence of platelet derived growth factor (PDGF) or a fragment thereof. The PDGF family consists of PDGF-A, -B, -C and -D, which form either homo- or heterodimers (PDGF-AA, -AB, -BB, -CC, -DD). In some embodiments, the PDGF is PDGFA. In some embodiments, the PDGF is a PDGFB. In some embodiments, the PDGF is a PDGFC. In some embodiments, the PDGF is a PDGFD. As used herein, “PDGFb” refers to the platelet derived growth factor subunit b (Pdgfb) gene or PDGFb protein of the family comprised of both platelet-derived growth factors (PDGF) and vascular endothelial growth factors (VEGF). The encoded preproprotein is proteolytically processed to generate platelet-derived growth factor subunit B, which can homodimerize, or alternatively, heterodimerize with the related platelet-derived growth factor subunit A. PDGFb can bind and activate PDGF receptor tyrosine kinases, which play a role in a wide range of developmental processes, angiogenesis, cell proliferation, and differentiation. PDGFb plays an essential role in the regulation of embryonic development, cell proliferation, cell migration, survival and chemotaxis. PDGFb is also a potent mitogen for cells of mesenchymal origin; required for normal proliferation and recruitment of pericytes and vascular smooth muscle cells in the central nervous system, skin, lung, heart and placenta; is required for normal blood vessel development, and for normal development of kidney glomeruli; plays an important role in wound healing.

In some embodiments, the cells are modified to overexpress the coding sequence of an PDGFb protein. In some embodiments, the cells are genetically modified and carry stable integrations of one or more copies of an PDGFb coding sequence. In some embodiments, the cells overexpress the coding sequence of PDGFb protein at levels sufficient to increase production and/or secretion of PDGFb into the cell culture medium.

In some embodiments, the PDGFb coding sequence is selected from any metazoan species. In some embodiments, the PDGFb coding sequence is from any animal, such as vertebrate and invertebrate animal species. In some embodiments, the PDGFb coding sequence is from any vertebrate animal species such as mammals, reptiles, birds, amphibians, and the like. In some embodiments, the PDGFb coding sequence is from any mammalian species such as a human, murine, bovine, porcine, poultry, and the like. In some embodiments, the coding sequence of the PDGFb protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus.

In some embodiments, increasing expression of PDGFb may be achieved using different approaches. In some embodiments, the expression is inducible. In some embodiments, the method comprises expressing polynucleotides comprising the coding sequence of PDGFb. In some embodiments, the polynucleotides are ectopically expressed from constructs that are introduced into the cells, for example expressed from a plasmid, or other expression vector. In some embodiments, the constructs are integrated into the cell's genome, and the expression is driven in that manner (e.g., PhiC31 Integration Systems). In some embodiments, the expression of the PDGFb gene involves electroporating a DNA, delivering a DNA complexed with a transfection vehicle, using a viral vector (e.g. retrovirus, lentivirus, adenovirus, adeno-associated virus,), and the like, or combinations thereof. In some embodiments, the expression is constitutive. In some embodiments, the expression is conditional (e.g. inducible).

In the methods described herein, a polynucleotide comprising a coding sequence of PDGFb may encode any homolog of PDGFb, including PDGFb paralogs, such as PDGFa, or any other PDGFb paralogs, or an PDGFb protein translated from any splice variants of an PDGFb gene, or may comprise any mutations in the PDGFb gene sequence including, but not limited to nucleotide deletions, truncations, fusions, or substitutions. Mutations may be synthetic or naturally occurring.

In some embodiments, PDGFB refers to the Pdgfb gene or PDGFB protein, or fragment or variant thereof (e.g., a PDGFB protein having one or more (e.g., one, two, three, four, five, six, seven, eight, nine, ten, or more amino acid substitutions, deletions or insertions as compared to a wild type PDGFB polypeptide)). FIG. 2C shows a sequence alignment for PDGFb from chicken, bovine, and human. In some embodiments, PDGFb refers to a sequence listed in FIG. 2C.

In some embodiments, a PDGFb protein comprises an amino acid sequence having at least 80% (e.g., at least 85%, 90%, 95%, 96%, 97%, 98%, or 99%) sequence identity to a sequence selected from SEQ ID NOs: 18-21. In some embodiments, the PDGFb protein sequence comprises an amino acid sequence selected from SEQ ID NOs: 18-21.

In some embodiments, introducing the polynucleotide comprising the coding sequence of the PDGFb protein alone is not sufficient to confer reduced reliance on exogenous growth factors on the cell line. In such embodiments, one or more additional growth factor ligands can be introduced into the cell line. For example, a polynucleotide comprising the coding sequence of a FGF2 protein or an IGF-1 protein can be introduced into the cell line to help confer reduced reliance on exogenous growth factors. In other embodiments, a polynucleotide comprising a coding sequence of a growth factor receptor can be introduced into the cell line to help confer reduced reliance on exogenous growth factors. In such embodiments, the growth factor receptor can be selected from a FGF2R, an IGF1R, and a PDFGR. For example, in order to confer reduced reliance on exogenous growth factors on the cell line, a polynucleotide comprising a coding sequence of a growth factor ligand (e.g., PDGFb) or a fragment thereof, and a polynucleotide comprising a coding sequence of a growth factor receptor (e.g., PDGFR) are introduced into the cell line.

### 6.3. Growth Factor Receptors and Activated Downstream Targets

Provided herein are methods for introducing into a cell a polynucleotide comprising a coding sequence of a growth factor receptor and/or an activated downstream growth factor target. In some embodiments, introducing the polynucleotide comprising the coding sequence of the growth factor receptor (and/or an activated downstream growth factor target) into the cells results in the cells having reduced reliance on exogenous growth factors.

In some embodiments, the growth factor receptor is selected from fibroblast growth factor receptor (FGFR), insulin growth factor 1 receptor (IGF1R), and platelet-derived growth factor receptor (PDGFR). In some embodiments, the method include introducing an additional two or more growth factor receptors into the cell, wherein each additional growth factor receptor is selected from FGFR, IGF1R, and PDGFR.

In some embodiments, the methods provided herein include introducing into a cell a polynucleotide comprising a coding sequence of a growth factor ligand and a polynucleotide comprising a coding sequence of a growth factor receptor. In such cases, the cells are engineered to overexpress the coding sequence of the growth factor ligand, the growth factor receptor, or both. In some embodiments, the growth factor ligand and the growth factor receptor are components of the same signaling axis, for example, FGF2 and FGF1/2/3/4; IGF1 and IGF1R; or PDGFb and PDGFR. In such cases, the overexpressing the growth factor ligand and growth factor receptor results in a synergistic effect on the results described herein. Without wishing to be bound by theory, overexpressing both a growth factor ligand and a growth factor receptor enables a cell (or population of cells) to circumvent feedback mechanisms used by the cell to regulate signaling, thereby driving continued signaling within a cell. For example, the cell (or population of cells) can circumvent the feedback loop by expressing and secreting more growth factor ligands, expressing more receptors at the surface of the cell, or by expressing constitutively active receptors.

In some embodiments, the cells are engineered to overexpress the coding sequence of the growth factor receptor. In some embodiments, the cells are genetically engineered to have stable integration of the one or more copies of a coding sequence for a growth factor receptor. In some embodiments, the cells overexpress the coding sequence of the growth factor receptor at levels sufficient to reduce reliance on exogenous growth factors.

In some embodiments, introducing the polynucleotide comprising the coding sequence of the activated downstream growth factor target into the cells results in the cells having reduced reliance on exogenous growth factors. In some embodiments, the activated downstream growth factor target comprises a growth factor receptor comprising one or more amino acid insertions, deletions, or substitutions that result in the receptor being constitutively activated. In some embodiments, the activated downstream growth factor target includes a mutation in the intracellular portion or the growth factor receptor that triggers a signaling cascade (e.g., a phosphorylation cascade). In some embodiments, the activated downstream growth factor target comprises a gene and/or target associated with FGF signaling, IGF1 signaling, or PDGF signaling.

### 6.3.1. Fibroblast Growth Factor Receptor

In some embodiments, the methods provided herein include introducing into a cell a polynucleotide comprising a coding sequence of fibroblast growth factor receptor (FGFR). As used herein, the term “FGFR” refers to fibroblast growth factor receptor (Fgfr) gene or FGFR protein that is a family where amino acid sequence is highly conserved between members and throughout evolution. FGFR family members differ from one another in their ligand affinities and tissue distribution. The extracellular portion of the protein interacts with fibroblast growth factors, setting in motion a cascade of downstream signals, ultimately influencing mitogenesis and differentiation. The FGFR family include from FGFR1, FGFR2, FGFR3, and FGFR4.

In some embodiments, the cells are modified to overexpress the coding sequence of an FGFR. In some embodiments, the cells are genetically modified and carry stable integrations of one or more copies of an FGFR coding sequence. In some embodiments, the cells overexpress the coding sequence of FGFR protein at levels sufficient to increase expression of FGFR at the surface of the cell.

In some embodiments, the FGFR coding sequence is selected from any metazoan species. In some embodiments, the FGFR coding sequence is from any animal, such as vertebrate and invertebrate animal species. In some embodiments, the FGFR coding sequence is from any vertebrate animal species such as mammals, reptiles, birds, amphibians, and the like. In some embodiments, the FGFR coding sequence is from any mammalian species such as a human, murine, bovine, porcine, poultry, and the like. In some embodiments, the coding sequence of the FGFR is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus.

In some embodiments, increasing expression of FGFR may be achieved using different approaches. In some embodiments, the expression is inducible. In some embodiments, the method comprises expressing polynucleotides comprising the coding sequence of FGFR. In some embodiments, the polynucleotides are ectopically expressed from constructs that are introduced into the cells, for example expressed from a plasmid, or other expression vector. In some embodiments, the constructs are integrated into the cell's genome, and the expression is driven in that manner (e.g., PhiC31 Integration Systems). In some embodiments, the expression of the FGFR gene involves electroporating a DNA, delivering a DNA complexed with a transfection vehicle, using a viral vector (e.g. retrovirus, lentivirus, adenovirus, adeno-associated virus), and the like, or combinations thereof. In some embodiments, the expression is constitutive. In some embodiments, the expression is conditional (e.g. inducible).

In some embodiments, a polynucleotide comprising a coding sequence of FGFR may encode any homolog of FGFR, including FGFR paralogs, such as FGFR1, FGFR2, FGFR3, and FGFR4, or any other FGFR paralogs, or an FGFR protein translated from any splice variants of an FGFR gene, or may comprise any mutations in the FGFR gene sequence including, but not limited to nucleotide deletions, truncations, fusions, or substitutions. Mutations may be synthetic or naturally occurring.

In some embodiments, FGFR refers to the FGFR protein, or fragment or variant thereof (e.g., a FGFR protein having one or more (e.g., one, two, three, four, five, six, seven, eight, nine, ten, or more amino acid substitutions, deletions or insertions as compared to a wild type FGFR protein)).

In some embodiments, a FGFR protein comprises an amino acid sequence having at least 80% (e.g., at least 85%, 90%, 95%, 96%, 97%, 98%, or 99%) sequence identity to a sequence selected from SEQ ID NOs: 32-49. In some embodiments, the FGFR protein sequence comprises an amino acid sequence selected from SEQ ID NOs: 32-49.

In some embodiments, introducing the polynucleotide comprising the coding sequence of the FGFR protein alone is not sufficient to confer reduced reliance on exogenous growth factors on the cell line. In such embodiments, one or more additional growth factor receptors can be introduced into the cell line. For example, a polynucleotide comprising the coding sequence of a PDGFR protein or an IGF-1R protein can be introduced into the cell line to help confer reduced reliance on exogenous growth factors. In other embodiments, a polynucleotide comprising a coding sequence of a growth factor ligand can be introduced into the cell line to help confer reduced reliance on exogenous growth factors. In such embodiments, the growth factor ligand can be selected from FGF2, IGF-1, and PDFG. For example, in order to confer reduced reliance on exogenous growth factors on the cell line, a polynucleotide sequence comprising a coding sequence of a growth factor receptor (e.g., FGFR) or a fragment thereof, and a polynucleotide comprising a coding sequence of a growth factor ligand (e.g., FGF2) are introduced into the cell line.

### 6.3.2. Insulin Growth Factor Receptor

In some embodiments, the methods provide herein include introducing into a cell a polynucleotide comprising a coding sequence of insulin-like growth factor receptor (IGFR). In some embodiments, the IGFR is an insulin like growth factor −1 receptor (IGF1R). As used herein, “IGFR1” refers to the insulin-like growth factor receptor (Igfr1) gene or IGF1R belonging to the class of tyrosine kinase receptors. IGF1R mediates the effects of IGF1, which is a polypeptide protein hormone similar in molecular structure to insulin.

In some embodiments, the cells are modified to overexpress the coding sequence of an IGF1R protein. In some embodiments, the cells are genetically modified and carry stable integrations of one or more copies of an IGF1R coding sequence. In some embodiments, the cells overexpress the coding sequence of IGF1R protein at levels sufficient to increase expression of IGF1R at the surface of the cell.

In some embodiments, the IGF1R coding sequence is selected from any metazoan species. In some embodiments, the IGF1R coding sequence is from any animal, such as vertebrate and invertebrate animal species. In some embodiments, the IGF1R coding sequence is from any vertebrate animal species such as mammals, reptiles, birds, amphibians, and the like. In some embodiments, the IGFIR coding sequence is from any mammalian species such as a human, murine, bovine, porcine, poultry, and the like. In some embodiments, the coding sequence of the IGF1R protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus.

In some embodiments, increasing expression of IGF1R may be achieved using different approaches. In some embodiments, the expression is inducible. In some embodiments, the method comprises expressing polynucleotides comprising the coding sequence of IGF1R. In some embodiments, the polynucleotides are ectopically expressed from constructs that are introduced into the cells, for example expressed from a plasmid, or other expression vector. In some embodiments, the constructs are integrated into the cell's genome, and the expression is driven in that manner (e.g., PhiC31 Integration Systems). In some embodiments, the expression of the IGF1R gene involves electroporating a DNA, delivering a DNA complexed with a transfection vehicle, using a viral vector (e.g. retrovirus, lentivirus, adenovirus, adeno-associated virus), and the like, or combinations thereof. In some embodiments, the expression is constitutive. In some embodiments, the expression is conditional (e.g. inducible).

In the methods described herein, a polynucleotide comprising a coding sequence of IGFR may encode any homolog of IGF1R, including IGF1R paralogs, such as IGF1R, INSR, INSRR, ROS1, ERBB4, EPHA, or any other IGF1R paralogs, or an IGF1R protein translated from any splice variants of an IGF1 gene, or may comprise any mutations in the IGF1R gene sequence including, but not limited to nucleotide deletions, truncations, fusions, or substitutions. Mutations may be synthetic or naturally occurring.

In some embodiments, IGF1R refers to the IGF1R protein, or fragment or variant thereof (e.g., a IGF1R protein having one or more (e.g., one, two, three, four, five, six, seven, eight, nine, ten, or more amino acid substitutions, deletions or insertions as compared to a wild type IGF1R protein)).

In some embodiments, a IGF1R protein comprises an amino acid sequence having at least 80% (e.g., at least 85%, 90%, 95%, 96%, 97%, 98%, or 99%) sequence identity to a sequence selected from SEQ ID NOs: 50-51. In some embodiments, the IGF1R protein sequence comprises an amino acid sequence selected from SEQ ID NOs: 50-51.

In some embodiments, introducing the polynucleotide comprising the coding sequence of the IGF1R protein alone is not sufficient to confer reduced reliance on exogenous growth factors on the cell line. In such embodiments, one or more additional growth factor receptors can be introduced into the cell line. For example, a polynucleotide comprising the coding sequence of a FGFR protein or an PDGFR protein can be introduced into the cell line to help confer reduced reliance on exogenous growth factors. In other embodiments, a polynucleotide comprising a coding sequence of a growth factor ligand can be introduced into the cell line to help confer reduced reliance on exogenous growth factors. In such embodiments, the growth factor ligand can be selected from FGF2, IGF1, and PDFG. For example, in order to confer reduced reliance on exogenous growth factors on the cell line, a polynucleotide sequence comprising a coding sequence of a growth factor receptor (e.g., IGF1R) or a fragment thereof, and a polynucleotide comprising a coding sequence of a growth factor ligand (e.g., IGF1) are introduced into the cell line.

### 6.3.3. Platelet Derived Growth Factor Receptor

In some embodiments, the methods provide herein include introducing into a cell a polynucleotide comprising a coding sequence of platelet derived growth factor receptor (PDGFR). As used herein, “PDGFR” refers to the platelet derived growth factor receptor (Pdgfr) gene or PDGFR protein of the protein family comprised of both platelet-derived growth factors (PDGF) and vascular endothelial growth factors (VEGF). PDGFRs are catalytic receptors that have intracellular tyrosine kinase activity. PDGFR have roles in the regulation of many biological processes including embryonic development, angiogenesis, cell proliferation and differentiation.

In some embodiments, the cells are modified to overexpress the coding sequence of an PDGFR protein. In some embodiments, the cells are genetically modified and carry stable integrations of one or more copies of an PDGFR coding sequence. In some embodiments, the cells overexpress the coding sequence of PDGFR protein at levels sufficient to increase expression of FGFR at the surface of the cell.

In some embodiments, the PDGFR coding sequence is selected from any metazoan species. In some embodiments, the PDGFR coding sequence is from any animal, such as vertebrate and invertebrate animal species. In some embodiments, the PDGFR coding sequence is from any vertebrate animal species such as mammals, reptiles, birds, amphibians, and the like. In some embodiments, the PDGFR coding sequence is from any mammalian species such as a human, murine, bovine, porcine, poultry, and the like. In some embodiments, the coding sequence of the PDGFR protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus.

In some embodiments, increasing expression of PDGFR may be achieved using different approaches. In some embodiments, the expression is inducible. In some embodiments, the method comprises expressing polynucleotides comprising the coding sequence of PDGFR. In some embodiments, the polynucleotides are ectopically expressed from constructs that are introduced into the cells, for example expressed from a plasmid, or other expression vector. In some embodiments, the constructs are integrated into the cell's genome, and the expression is driven in that manner (e.g., PhiC31 Integration Systems). In some embodiments, the expression of the PDGFR gene involves electroporating a DNA, delivering a DNA complexed with a transfection vehicle, using a viral vector (e.g. retrovirus, lentivirus, adenovirus, adeno-associated virus), and the like, or combinations thereof. In some embodiments, the expression is constitutive. In some embodiments, the expression is conditional (e.g. inducible).

In the methods described herein, a polynucleotide comprising a coding sequence of PDGFR may encode any homolog of PDGFR, including PDGFR paralogs, such as PDGFRA and PDGFRB, or any other PDGFR paralogs, or an PDGFR protein translated from any splice variants of an PDGFR gene, or may comprise any mutations in the PDGFR gene sequence including, but not limited to nucleotide deletions, truncations, fusions, or substitutions. Mutations may be synthetic or naturally occurring.

In some embodiments, PDGFR refers to the PDGFR protein, or fragment or variant thereof (e.g., a PDGFR protein having one or more (e.g., one, two, three, four, five, six, seven, eight, nine, ten, or more amino acid substitutions, deletions or insertions as compared to a wild type PDGFR protein)).

In some embodiments, a PDGFR protein comprises an amino acid sequence having at least 80% (e.g., at least 85%, 90%, 95%, 96%, 97%, 98%, or 99%) sequence identity to a sequence selected from SEQ ID NOs: 52-58. In some embodiments, the PDGFR protein sequence comprises an amino acid sequence selected from SEQ ID NOs: 52-58.

In some embodiments, introducing the polynucleotide comprising the coding sequence of the PDGFRB protein alone is not sufficient to confer reduced reliance on exogenous growth factors on the cell line. In such embodiments, one or more additional growth factor receptors can be introduced into the cell line. For example, a polynucleotide comprising the coding sequence of a FGFR protein or an IGF1R protein can be introduced into the cell line to help confer reduced reliance on exogenous growth factors. In other embodiments, a polynucleotide comprising a coding sequence of a growth factor ligand can be introduced into the cell line to help confer reduced reliance on exogenous growth factors. In such embodiments, the growth factor ligand can be selected from FGF2, IGF1, and PDFG. For example, in order to confer reduced reliance on exogenous growth factors on the cell line, a polynucleotide sequence comprising a coding sequence of a growth factor receptor (e.g., PDGFRB) or a fragment thereof; and a polynucleotide comprising a coding sequence of a growth factor ligand (e.g., PDGF) are introduced into the cell line.

### 6.4. Accessory Proteins

In some embodiments, the methods provided herein include introducing into a cell a polynucleotide comprising a coding sequence of an accessory protein.

### 6.4.1. Fibroblast Growth Factor Binding Protein

In some embodiments, accessory protein refers to a fibroblast growth factor binding protein (FGFBP). As used herein, “FGFBP” refers to the fibroblast growth factor binding protein (Fgfbp) gene or FGFBP protein, which belongs to a family of fibroblast growth factor carrier proteins. Family members include, without limitation, FGFBP1, FGFBP2, and FGFBP3. FGFBPs play a role in cell proliferation, differentiation and migration by binding to fibroblast growth factors and potentiating their biological effects on target cells.

In some embodiments, the FGFBP is FGBP1 or a fragment thereof. In some embodiments, FGFBP1 enhances FGF2 signaling.

In some embodiments, FGFBP1 refers to the Fgfbp1 gene or FGFBP1 protein, or fragment or variant thereof (e.g., a FGFBP1 protein having one or more (e.g., one, two, three, four, five, six, seven, eight, nine, ten, or more amino acid substitutions, deletions or insertions as compared to a wild type FGFBP1 polypeptide)).

In some embodiments, a FGFBP1 protein comprises an amino acid sequence having at least 80% (e.g., at least 85%, 90%, 95%, 96%, 97%, 98%, or 99%) sequence identity to a sequence selected from SEQ ID NOs: 59. In some embodiments, the FGFBP1 protein sequence comprises an amino acid sequence selected from SEQ ID NOs: 59.

### 6.4.2. RASV12

In some embodiments, accessory protein refers to a H-RasV12 or RASV12. As used herein, “RASV12” refers to the H-RasV12 protein or RASV12 protein. RASV12 is involved in the activation of Ras protein signal transduction and binds GDP/GTP and possess intrinsic GTPase activity.

In some embodiments, RASV12 refers to the RASV12 protein, or fragment or variant thereof (e.g., a RASV12 protein having one or more (e.g., one, two, three, four, five, six, seven, eight, nine, ten, or more amino acid substitutions, deletions or insertions as compared to a wild type RASH or RASV12 polypeptide)).

In some embodiments, a RASV12 protein comprises an amino acid sequence having at least 80% (e.g., at least 85%, 90%, 95%, 96%, 97%, 98%, or 99%) sequence identity to a sequence selected from SEQ ID NOs: 60. In some embodiments, the RASV12 protein sequence comprises an amino acid sequence selected from SEQ ID NOs: 60.

### 6.5. Signal Peptides

In some embodiments, the methods provided herein include introducing into a cell a polynucleotide comprising a coding sequence of a growth factor ligand, where the growth factor ligand is fused to a signal peptide. In some embodiments, the polynucleotide includes a sequence encoding a signal peptide located 5′ to the coding sequence of the growth factor ligand, and wherein the signal sequence and the growth factor ligand are a fusion protein. In some embodiments, the polynucleotide includes a sequence encoding a signal peptide located 3′ to the coding sequence of the growth factor ligand, and wherein the signal sequence and the growth factor ligand are a fusion protein. Non-limiting examples of signal peptides having at least 80% (e.g., at least 85%, 90%, 95%, 96%, 97%, 98%, or 99%) sequence identity to a sequence selected from SEQ ID NOs: 22-31.

### 6.6. Method for Increasing Concentration of a Growth Factor in Culture Medium

Provided herein are methods of increasing the concentration of a growth factor ligand in culture medium of cells in culture where the method includes introducing one or more of a coding sequence of any of the growth factor ligands described herein, a coding sequence of any of the growth factor receptors described herein, a coding sequence of any of the activated downstream growth factors described herein, a coding sequence of any of the accessory proteins described herein, or a combination thereof, into the cell line; and culturing the cell line in a cultivation infrastructure.

In some embodiments, introducing a polynucleotide comprising a coding sequence of FGF2 into a cell line and culturing the cells in a cultivating infrastructure results in an increase of FGF2 in the medium of the cells in culture.

In some embodiments, the concentration of FGF2 in the culture medium is increased by at least 0.00001 ng/mL, (e.g., at least 0.000025 ng/mL, at least 0.000075 ng/mL, by at least 0.0005 ng/mL, at least 0.001 ng/mL, at least 0.005 ng/mL, at least 0.01 ng/mL, at least 0.05 ng/mL, at least 0.1 ng/mL, at least 0.5 ng/mL, at least 1.0 ng/mL, at least 2.5 ng/mL, at least 5.0 ng/mL, at least 7.5 ng/mL, at least 10 ng/mL, at least 15 ng/mL, at least 20 ng/mL, at least 25 ng/mL, at least 30 ng/mL, at least 40 ng/mL, at least 50 ng/mL, at least 60 ng/mL, at least 70 ng/mL, at least 80 ng/mL, at least 90 ng/mL, at least 100 ng/mL, at least 150 ng/mL, at least 200 ng/mL, at least 200 ng/mL, at least 250 ng/mL, at least 300 ng/mL, at least 350 ng/mL, at least 400 ng/mL, at least 450 ng/mL, at least 500 ng/mL, at least 550 ng/mL, at least 600 ng/mL, at least 650 ng/mL, at least 700 ng/mL, at least 750 ng/mL, at least 800 ng/mL, at least 850 ng/mL, at least 900 ng/mL, at least 950 ng/mL, or at least 1000 ng/mL) as compared to a cell line not engineered to include a polynucleotide comprising a coding sequence of FGF2.

In some embodiments, the concentration of FGF2 in the culture medium is increased by at least 0.01% (e.g., at least 0.05%, at least 0.1%, at least 0.5%, at least 1.0%, at least 1.5%, at least 2.0%, at least 2.5%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, at least 1,100%, at least 1,200%, at least 1,300%, at least 1,400%, at least 1,500%, at least 1,600%, at least 1,700%, at least 1,800%, at least 1,900%, at least 2,000%, at least 2,250%, at least 2,500%, at least 2,750%, at least 3,000%, at least 3,500%, at least 4,000%, at least 4,500%, at least 5,000%, at least 6,000%, at least 7,000%, at least 8,000%, at least 9,000%, or at least 10,000%) as compared to a cell line not engineered to include a polynucleotide encoding FGF2.

In some embodiments, introducing a polynucleotide comprising a coding sequence of IGF1 into a cell line and culturing the cells in a cultivating infrastructure results in an increase of IGF1 in the medium of the cells in culture.

In some embodiments, the concentration of IGF1 in the culture medium is increased by at least 0.00001 ng/mL, (e.g., at least 0.000025 ng/mL, at least 0.000075 ng/mL, at least 0.0005 ng/mL, at least 0.001 ng/mL, at least 0.005 ng/mL, at least 0.01 ng/mL, at least 0.05 ng/mL, at least 0.1 ng/mL, at least 0.5 ng/mL, at least 1.0 ng/mL, at least 2.5 ng/mL, at least 5.0 ng/mL, at least 7.5 ng/mL, at least 10 ng/mL, at least 15 ng/mL, at least 20 ng/mL, at least 25 ng/mL, at least 30 ng/mL, at least 40 ng/mL, at least 50 ng/mL, at least 60 ng/mL, at least 70 ng/mL, at least 80 ng/mL, at least 90 ng/mL, at least 100 ng/mL, at least 150 ng/mL, at least 200 ng/mL, at least 200 ng/mL, at least 250 ng/mL, at least 300 ng/mL, at least 350 ng/mL, at least 400 ng/mL, at least 450 ng/mL, at least 500 ng/mL, at least 550 ng/mL, at least 600 ng/mL, at least 650 ng/mL, at least 700 ng/mL, at least 750 ng/mL, at least 800 ng/mL, at least 850 ng/mL, at least 900 ng/mL, at least 950 ng/mL, or at least 1000 ng/mL) as compared to a cell line not engineered to include a polynucleotide encoding IGF-1.

In some embodiments, the concentration of IGF1 in the culture medium is increased by at least 0.01% (e.g., at least 0.05%, at least 0.1%, at least 0.5%, at least 1.0%, at least 1.5%, at least 2.0%, least 2.5%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, at least 1,100%, at least 1,200%, at least 1,300%, at least 1,400%, at least 1,500%, at least 1,600%, at least 1,700%, at least 1,800%, at least 1,900%, at least 2,000%, at least 2,250%, at least 2,500%, at least 2,750%, at least 3,000%, at least 3,500%, at least 4,000%, at least 4,500%, at least 5,000%, at least 6,000%, at least 7,000%, at least 8,000%, at least 9,000%, or by at least 10,000%) as compared to a cell line not engineered to include a polynucleotide encoding IGF1.

In some embodiments, introducing a polynucleotide comprising a coding sequence of PDGFb into a cell line and culturing the cells in a cultivating infrastructure results in an increase of PDGFb in the medium of the cells in culture.

In some embodiments, the concentration of PDGFb in the culture medium is increased by at least 0.00001 ng/mL, (e.g., at least 0.000025 ng/mL, at least 0.000075 ng/mL, at least 0.0005 ng/mL, at least 0.001 ng/mL, at least 0.005 ng/mL, at least 0.01 ng/mL, at least 0.05 ng/mL, at least 0.1 ng/mL, at least 0.5 ng/mL, at least 1.0 ng/mL, at least 2.5 ng/mL, at least 5.0 ng/mL, at least 7.5 ng/mL, at least 10 ng/mL, at least 15 ng/mL, at least 20 ng/mL, at least 25 ng/mL, at least 30 ng/mL, at least 40 ng/mL, at least 50 ng/mL, at least 60 ng/mL, at least 70 ng/mL, at least 80 ng/mL, at least 90 ng/mL, at least 100 ng/mL, at least 150 ng/mL, at least 200 ng/mL, at least 200 ng/mL, at least 250 ng/mL, at least 300 ng/mL, at least 350 ng/mL, at least 400 ng/mL, at least 450 ng/mL, at least 500 ng/mL, at least 550 ng/mL, at least 600 ng/mL, at least 650 ng/mL, at least 700 ng/mL, at least 750 ng/mL, at least 800 ng/mL, at least 850 ng/mL, at least 900 ng/mL, at least 950 ng/mL, or at least 1000 ng/mL) as compared to a cell line not engineered to include a polynucleotide encoding PDGFb. [0265]) In some embodiments, the concentration of PDGFb in the culture medium is increased by at least 0.01% (e.g., at least 0.05%, at least 0.1%, at least 0.5%, at least 1.0%, at least 1.5%, at least 2.0%, least 2.5%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, at least 1,100%, at least 1,200%, at least 1,300%, at least 1,400%, at least 1,500%, at least 1,600%, at least 1,700%, at least 1,800%, at least 1,900%, at least 2,000%, at least 2,250%, at least 2,500%, at least 2,750%, at least 3,000%, at least 3,500%, at least 4,000%, at least 4,500%, at least 5,000%, at least 6,000%, at least 7,000%, at least 8,000%, at least 9,000%, or at least 10,000%) as compared to a cell line not engineered to include a polynucleotide encoding PDGFb.

The methods of the present disclosure advantageously enable selection of transfected cells using only the absence of growth factors, thereby obviating the need for antibiotic and cre-lox selection protocols, which are less desirable when creating a food product since they are not recognized as generally acceptable for consumption.

### 6.7. Method of Increasing Cell Density

Provided herein are methods of increasing the cell density of a culture where the method includes introducing one or more of a coding sequence of any of the growth factor ligands described herein, a coding sequence of any of the growth factor receptors described herein, a coding sequence of any of the activated downstream growth factor targets described herein, a coding sequence of any of the accessory proteins described herein, or a combination thereof, into the cell line; and culturing the cell line in a cultivation infrastructure.

In some embodiments, an increase in the cell density of a culture (e.g., suspension culture) using the methods described herein is about 1.025 fold, 1.05 fold, 1.10-fold, 1.15-fold, 1.20-fold, 1.25-fold, 1.30 fold, 1.35-fold, 1.40-fold, 1.45-fold, 1.5-fold, 2-fold, 2.5-fold, 3-fold, 3.5-fold, 4-fold, 4.5-fold, 5-fold, 7.5-fold, 10-fold, 15-fold, 20-fold, 25-fold, 30-fold, 40-fold, or even about 50-fold, 75-fold, 100-fold, 150-fold, or about 200-fold, compared to the density of a culture comprising cells that do not include a coding sequence of any of the growth factor ligands described herein, a coding sequence of any of the growth factor receptors described herein, a coding sequence of any of the accessory proteins described herein, or a combination thereof.

In some embodiments, an increase in the density of cells in a culture (e.g., suspension culture) using the methods described herein is at least 10%, at least 15%, at least 20%, at least 25%, at least 30%, at least 35%), at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, compared to the density of a culture comprising cells that do not include a coding sequence of any of the growth factor ligands described herein, a coding sequence of any of the growth factor receptors described herein, a coding sequence of any of the accessory proteins described herein, or a combination thereof.

In some embodiments, methods described herein increase the density of cells in a culture (e.g., suspension culture) by increasing the rate of proliferation of cells in the culture. In some embodiments, the increase in the rate of cell proliferation is at least 2.5%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, or at least 1000%), including values and ranges therebetween, compared to the density of a culture comprising cells that do not include a coding sequence of any of the growth factor ligands described herein, a coding sequence of any of the growth factor receptors described herein, a coding sequence of any of the accessory proteins described herein, or a combination thereof. In some embodiments, the increase in the rate of cell proliferation is about 25-1000%, about 25-750%, about 25-500%, about 50-1000%, about 50-750%, about 50-500%, about 100-1000%, about 100-750%, or about 100-500%, including values and ranges therebetween, compared to the density of a culture comprising cells that do not include a coding sequence of any of the growth factor ligands described herein, a coding sequence of any of the growth factor receptors described herein, a coding sequence of any of the accessory proteins described herein, or a combination thereof.

In some embodiments, methods described herein increase the cell density of a culture (e.g., suspension culture) by decreasing cell death within the cellular biomass. In some embodiments, the decrease in cell death is at least 2.5%, at least 5%, at least 10%>, at least 15%, at least 20%, at least 25%, at least 30%), at least 35%, at least 40%, at least 45%, at least 50%, at least 55%, at least 60%, at least 65%, at least 70%, at least 75%, at least 80%, at least 85%, at least 90%, at least 95%, at least 100%), including values and ranges therebetween, compared to the density of a culture comprising cells that do not include a coding sequence of any of the growth factor ligands described herein, a coding sequence of any of the growth factor receptors described herein, a coding sequence of any of the accessory proteins described herein, or a combination thereof. In some embodiments, a decrease in the rate of cell death within the cellular biomass is about 2.5-10%, about 2.5-75%, about 2.5-50%, about 5.0-100%, about 5.0-75%, about 5.0-50%, about 10-100%, about 10-75%, or about 10-50%, including values and ranges therebetween, compared to the density of a culture comprising cells that do not include a coding sequence of any of the growth factor ligands described herein, a coding sequence of any of the growth factor receptors described herein, a coding sequence of any of the accessory proteins described herein, or a combination thereof.

In some embodiments, using the methods described herein, the density of cells in a culture may reach about 1E4 cells/mL, about 1E5 cells/mL, about 1E6 cells/mL, about 1E7 cells/mL, about 1E8 cells/mL, about 1E9 cells/mL, about 1E10 cells/mL, about 1E11 cells/mL, about 1E12 cells/mL, or about 1E13 cells/mL (cells in suspension culture or cells in the cellular biomass/mL of cultivation infrastructure), including values and ranges therebetween.

In some embodiments, using the methods described herein, the density of cells in a culture (e.g., suspension culture) may reach about 1 g/L, 5 g/L, 10 g/L, 25 g/L, 50 g/L, 75 g/L, 100 g/L, 150 g/L, 200 g/L, 250 g/L, 300 g/L, 350 g/L, 400 g/L, 450 g/L, 500 g/L, 550 g/L, 600 g/L, 650 g/L, 700 g/L, 750 g/L, 800 g/L, 850 g/L, 900 g/L, or 1000 g/L (g of cellular biomass/L of cultivation infrastructure), including values and ranges therebetween. In some embodiments, the density of cells in a culture (e.g., suspension culture) may range from about 1 g/L to about 5 g/L, about 1 g/L to about 750 g/L, about 1 g/L to about 500 g/L, about 1 g/L to about 250 g/L, about 1 g/L to about 100 g/L, about 1 g/L to about 50 g/L, about 5 g/L to about 1000 g/L, about 5 g/L to about 750 g/L, about 5 g/L to about 500 g/L, about 5 g/L to about 250 g/L, about 5 g/L to about 100 g/L, about 5 g/L to about 50 g/L, about 25 g/L to about 1000 g/L, about 25 g/L to about 750 g/L, about 25 g/L to about 500 g/L, about 25 g/L to about 300 g/L, about 25 g/L to about 250 g/L, about 25 g/L to about 100 g/L, about 50 g/L to about 1000 g/L, about 50 g/L to about 750 g/L, about 50 g/L to about 500 g/L, about 50 g/L to about 300 g/L, about 50 g/L to about 250 g/L, about 100 g/L to 1000 g/L, about 100 g/L to about 750 g/L, about 100 g/L to about 500 g/L, about 200 g/L to about 1000 g/L, about 200 g/L to about 750 g/L, about 200 g/L to about 500 g/L, about 300 g/L to about 1000 g/L, about 300 g/L to about 800 g/L, about 400 g/L to about 1000 g/L, or about 500 g/L to about 1000 g/L including values and ranges therebetween.

### 6.8. Method for Improving Anchorage Independent Growth

Provided herein are methods of improving anchorage independent growth in a cell line where the method includes introducing a coding sequence of any of the growth factor ligands described herein, a coding sequence of any of the growth factor receptors described herein, a coding sequence of any of the accessory protein described herein, or a combination thereof, into the cell line; and culturing the cell line in a cultivation infrastructure.

In some embodiments, a cell line's ability to grow as a non-adherent, anchorage independent cell line can be modulated by culturing the cell line with FGF2 and/or IGF1 growth factors. In some embodiments, introducing a polynucleotide comprising a coding sequence of FGF2 and/or IGF1 into a cell line results in a cell line with at least a portion of the cell line having anchorage independent growth. In some embodiments, introducing a polynucleotide comprising a coding sequence of an FGFR and/or an IGFR results in a cell line with at least a portion of the cell line having anchorage independent growth.

In some embodiments, introducing a polynucleotide comprising a coding sequence of FGF2 or a fragment thereof and/or a coding sequence of IGF1 or a fragment thereof results in an increase in the rate of cell proliferation and decrease in cell death, which promotes the cell line (e.g., an adherent cell line) to transition to a non-adherent form (e.g., a non-adherent cell line). In some embodiments, an increase in the rate of cell proliferation and decrease in cell death promotes transition to anchorage-independent growth from anchorage-dependent growth.

In some embodiments, increase in the rate of cell proliferation is at least 2.5%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, including values and ranges therebetween, compared to a cell line not including a polynucleotide comprising a coding sequence of FGF2 and/or a coding sequence of IGF1. In some embodiments, the increase in the rate of cell proliferation is about 25-1000%, about 25-750%, about 25-500%, about 50-1000%, about 50-750%, about 50-500%, about 100-1000%, about 100-750%, or about 100-500%, including values and ranges therebetween, compared to a cell line not including a polynucleotide comprising a coding sequence of FGF2 and/or a coding sequence of IGF1.

In some embodiments, the methods provided herein that include introducing a polynucleotide comprising a coding sequence of FGF2 or a fragment thereof and/or a coding sequence of IGF1 or a fragment thereof into a cell line promote anchorage-independent growth of the cell line by decreasing cell-to-cell contact inhibition. In some embodiments, the decrease in contact inhibition provided by the present methods is about 10%, 20%, 30%, 40%, 50%, 60%, 70%, 80%, 90%, or 100%, including values and ranges therebetween, compared to a cell line not including a polynucleotide comprising a coding sequence of FGF2 or a coding sequence of IGF1.

In some embodiments, the methods provided herein that include introducing a polynucleotide comprising a coding sequence of FGF2 or a fragment thereof and/or a coding sequence of IGF1 or a fragment thereof into a cell line promote anchorage-independent growth of the cell line by decreasing cell death. In some embodiments, the decrease in cell death provided by the present methods is about 2.5%, 5%, 10%, 20%, 30%, 40%, 50%, 60%, 70%, 80%, 90%, or 100%, including values and ranges therebetween, compared to a cell line not including a polynucleotide comprising a coding sequence of FGF2 or a coding sequence of IGF1.

In some embodiments, introducing a polynucleotide comprising a coding sequence of FGFR or a fragment thereof or a coding sequence of an IGF1R or a fragment thereof results in an increase in the rate of cell proliferation and decrease in cell death, which promotes cell line (e.g., an adherent cell line) to transition to a non-adherent form (e.g., a non-adherent cell line). In some embodiments, an increase in the rate of cell proliferation and decrease in cell death promotes transition to anchorage-independent growth from anchorage-dependent growth.

In some embodiments, increase in the rate of cell proliferation is at least 2.5%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, including values and ranges therebetween, compared to a cell line not including a polynucleotide comprising a coding sequence of IGF1. In some embodiments, the increase in the rate of cell proliferation is about 25-1000%, about 25-750%, about 25-500%, about 50-1000%, about 50-750%, about 50-500%, about 100-1000%, about 100-750%, or about 100-500%, including values and ranges therebetween, compared to a cell line not including a polynucleotide comprising a coding sequence of FGFR or a coding sequence of IGF1R.

In some embodiments, the methods provided herein that include introducing a polynucleotide comprising a coding sequence of FGFR or a fragment thereof or a coding sequence of an IGFR or a fragment thereof into a cell line promote anchorage-independent growth of the cell line by decreasing cell-to-cell contact inhibition. In some embodiments, the decrease in contact inhibition provided by the present methods is about 10%, 20%, 30%, 40%, 50%, 60%, 70%, 80%, 90%, or 100%, including values and ranges therebetween, compared to a cell line not including a polynucleotide comprising a coding sequence of FGFR or a coding sequence of IGF1R.

In some embodiments, the methods provided herein that include introducing a polynucleotide comprising a coding sequence of FGFR or a fragment thereof or a coding sequence of an IGFR or a fragment thereof into a cell line promote anchorage-independent growth of the cell line by decreasing cell death. In some embodiments, the decrease in cell death provided by the present methods is about 2.5%, 5%, 10%, 20%, 30%, 40%, 50%, 60%, 70%, 80%, 90%, or 100%, including values and ranges therebetween, compared to a cell line not including a polynucleotide comprising a coding sequence of FGFR or a coding sequence IGF1R.

### 6.9. Methods for Producing Cell-Based Meat Suitable for Consumption

Provided herein are in vitro methods for producing cell-based meat suitable for consumption, comprising: (a) introducing into a cell line one or more of a polynucleotide comprising a coding sequence of a growth factor ligand or a fragment thereof, a polynucleotide comprising a coding sequence of a growth factor receptor, a polynucleotide comprising a coding sequence of an activated downstream growth factor target; (b) inducing myogenic specific differentiation, wherein the differentiated cells form myotubes and multinucleated myotubes; (c) culturing the myocytes and myotubes to generate skeletal muscle fibers, thereby producing a cell-based meat suitable for consumption. In some embodiments, the in vitro method for producing cell-based meat suitable for consumption includes a step of adapting the cells to be grown in suspension. In some embodiments, the in vitro method for producing cell-based meat suitable for consumption includes a step of culturing the cells in a cultivation infrastructure.

In some embodiments, provided herein is cell-based meat suitable for consumption produced by the in vitro methods described herein.

In some embodiments, the in vitro method for producing cell-based meat suitable for consumption includes maintaining the engineered cell line in a culture medium comprising a molecule capable of controlling an inducible promoter, thereby enabling expression of the growth factor ligand (e.g., any of the growth factor ligands described herein), growth factor receptor (e.g., any of the growth factor receptors described herein), or a combination thereof, at specified times during the in vitro method. In some embodiments of the in vitro method, the engineered cell line is maintained in culture medium comprising a molecule capable of controlling an inducible promoter prior, contemporaneously with, or after the step of inducing myogenic specific differentiation. In some embodiments of the in vitro method, the engineered cell line is maintained in culture medium comprising a molecule capable of controlling an inducible promoter prior to the step of inducing myogenic specific differentiation. In such cases, the expression of the growth factor ligand, growth factor receptor, or a combination thereof, can be decreased or eliminated prior to the inducing of myogenic specific differentiation.

In some embodiments, the in vitro method for producing cell-based meat suitable for consumption includes maintaining the engineered cell line in a culture medium comprising a molecule capable of controlling the inducible tag, thereby inducing activity of the growth factor ligand/fusion protein (e.g., any of the growth factor ligands described herein), growth factor receptor/fusion protein (e.g., any of the growth factor receptors described herein), or a combination thereof, at specified times during the in vitro method. In some embodiments of the in vitro method, the engineered cell line is maintained in culture medium comprising a molecule capable of controlling an inducible tag prior, contemporaneously with, or after the step of inducing myogenic specific differentiation. In some embodiments of the in vitro method, the engineered cell line is maintained in culture medium comprising a molecule capable of controlling an inducible tag prior to the step of inducing myogenic specific differentiation. In such cases, the activity of the growth factor ligand, growth factor receptor, or a combination thereof, can be reduced or inhibited prior to the inducing of myogenic specific differentiation.

In some embodiments, the in vitro method for producing cell-based meat suitable for consumption includes maintaining the engineered cell line in a culture medium comprising a molecule controlling the degradation tag, thereby targeting the growth factor ligand/fusion protein (e.g., any of the growth factor ligands described herein), growth factor receptor/fusion protein (e.g., any of the growth factor receptors described herein), or a combination thereof, for degradation at specific times during the in vitro method. In some embodiments of the in vitro method, the engineered cell line is maintained in culture medium comprising a molecule capable of controlling a degradation tag prior, contemporaneously with, or after the step of inducing myogenic specific differentiation. In some embodiments of the in vitro method, the engineered cell line is maintained in culture medium comprising a molecule capable of controlling a degradation tag contemporaneously with or after the step of inducing myogenic specific differentiation. In such cases, the activity of the growth factor ligand, growth factor receptor, or combination thereof, can be reduced or inhibited in the same step as or shortly after beginning the inducing of myogenic specific differentiation.

In some embodiments, the cell line is from a livestock, poultry, game or aquatic animal species. In some embodiments, the cell line is from a chicken, duck, or turkey. In some embodiments, the cell line is from a fish. In some embodiments, the cell line is from a livestock species. In some embodiments, the livestock species is porcine or bovine. In some embodiments, the cells are from any animal species intended for human or non-human dietary consumption. In some embodiments, the cells are myogenic cells. In some embodiments, the myogenic cells are myoblasts, myocytes, satellite cells, side population cells, muscle derived stem cells, mesenchymal stem cells, myogenic pericytes, or mesoangioblasts. In some embodiments, the cells are non-myogenic cells.

Non-limiting examples of myogenic differentiation are as described in WO2019014652A1 and WO2015066377A1, both of which are herein incorporated by reference in their entireties.

In some embodiments, the myogenic cells and/or skeletal muscle produced according to the methods described herein can be processed as a raw, uncooked food product (cultured meat) or as a cooked food product or as a cooked/uncooked food ingredient. In some embodiments, processing comprises withdrawal of the culture medium that supports the viability, survival, growth or expansion (e.g., increase in total protein content of the non-naturally occurring myogenic cells) and differentiation of the myogenic cells. Withdrawal may comprise physical removal of the culture medium or altering the composition of the culture medium, for example, by addition of components that would reduce or prevent further expansion and/or differentiation of the cell line or cells-derived from the cell line or by depletion of components that support expansion and/or differentiation of the cell line or cells derived from the cell line.

### 6.9.1. Inducible Promoter, Inducible Tag, and Degradation Tag

In some embodiments, activity and/or expression of the growth factor ligand (e.g., any of the growth factor ligands described herein), growth factor receptor (e.g., any of the growth factor receptors described herein), or a combination thereof, is controllable. In some embodiments, control of the activity and/or expression of the growth factor ligand, growth factor receptor, or a combination thereof, is controlled using an inducible promoter, an inducible tag, and/or a degradation tag.

In some embodiments, expression of a growth factor ligand or a fragment thereof, growth factor receptor or a fragment thereof, or a combination thereof, is controlled using an inducible promoter where expression is induced in the presence of a molecule capable of inducing the inducible promoter. In some embodiments, the promoter is a positive inducible promoter (e.g., addition of a controllable molecule induces binding to the promoter thereby activating transcription). In some embodiments, the promoter is a negative inducible promoter (e.g., addition of a controllable molecule removes a repressor from the promoter thereby allowing transcriptional machinery to bind and activate transcription). Inducible promoters include, without limitation, chemically inducible promoters (e.g., tetracycline inducible (tetracycline response elements (TRE)/tetracycline activator), cumate inducible (cumate operator (CuO)/cumate activator), alcohol inducible (AlcA promoter/AlcR activator), and steroid-inducible (e.g., LexA promoter/XVE (synthetic) activator)); temperature inducible promoters (e.g., heat shock inducible (e.g., hsp70, hsp90), and light inducible promoters (e.g., FixK2 promoter/blue-light sensing protein YFI; Vivid (VVD)/light oxygen voltage (LOV)).

In some embodiments, the coding sequence of the growth factor ligand or a fragment thereof, growth factor receptor or a fragment thereof, or both are operably linked to the inducible promoter. To induce expression of the growth factor ligand, the growth factor receptor, or a combination thereof, the engineered cell line is maintained in a culture medium comprising a molecule capable of controlling the inducible promoter.

In some embodiments, activity of a growth factor ligand or a fragment thereof, a growth factor receptor or a fragment thereof, or a combination thereof, is controlled using an inducible tag where activity of the growth factor ligand and/or growth factor receptor is controlled by the presence or absence of a molecule capable of capable of controlling the inducible tag. In such embodiments, the polynucleotide includes a coding sequence of an inducible tag located 5′ or 3′ to the coding sequence of the growth factor ligand, and wherein the inducible tag and the growth factor ligand are a fusion protein. In such cases, the inducible tag prevents growth factor ligand activity in the absence of the controllable molecule. To enable activity of the growth factor ligand, the engineered cell line is maintained in a culture medium comprising a molecule capable of controlling the inducible tag, thereby controlling the activity of the fusion protein.

In some embodiments, the inducible tag is a ESR1 ligand binding domain that is activated in the presence of the ESR1 agonist (e.g., 17-β Estradiol (E2)), thereby allowing the growth factor ligand to bind to its receptor and facilitate downstream signaling. In some embodiments, the inducible tag is an estrogen receptor binding domain that is activated in the presence of tamoxifen, thereby allowing the growth factor ligand to bind to its receptor and facilitate downstream signaling.

In some embodiments, the activity of a growth factor ligand or a fragment thereof, a growth factor receptor or a fragment thereof, or a combination thereof, is controlled using a degradation tag, where activity of the growth factor ligand and/or growth factor receptor is controlled by the presence or absence of a molecule capable of controlling the degradation tag. In some embodiments, the polynucleotide includes a coding sequence of the degradation tag located 5′ or 3′ to the coding sequence of the growth factor ligand, and wherein the degradation tag and the growth factor ligand are a fusion protein. In such cases, the degradation tag targets the growth factor ligand for degradation upon addition of a molecule capable of controlling the degradation tag to the culture medium.

Non-limiting examples of degradation tags include: Anchor away (e.g., the FKBP12-rapamycin-binding-domain of FRAP (FRB) is fused to a specific protein of interest, while the FKBP12 is fused to the anchor protein, rapamycin bridges between the POI and the anchor protein, allowing functional inhibition of the target protein); auxin-inducible degron system; and dTag system (i.e., three major components: an FKBP12F36V-fused protein of interest, a small synthetic molecule, a defined degrader, and the endogenous E3 ligase complex).

### 6.10. Cultivation Infrastructure

In some embodiments, a cultivation infrastructure may be a tube, a cylinder, a flask, a petri-dish, a multi-well plate, a dish, a vat, a roller bottle, an incubator, a bioreactor, an industrial fermenter and the like.

In some embodiments, a cultivation infrastructure can be of any scale, and support any volume of cellular biomass and culturing reagents. In some embodiments, the cultivation infrastructure ranges from about 10 μL to about 100,000 L. In some embodiments, the cultivation infrastructure is about 10 μL, about 100 μL, about 1 mL, about 10 mL, about 100 mL, about 1 L, about 10 L, about 100 L, about 1000 L, about 10,000 L, or even about 100,000 L.

In some embodiments, the cultivation infrastructure comprises a substrate. In some embodiments, a cultivation infrastructure may comprise a permeable substrate (e.g. permeable to physiological solutions) or an impermeable substrate (e.g. impermeable to physiological solutions).

In some embodiments, the cultivation infrastructure comprises a primary substrate, which can be a flat, concave, or convex substrate. In some embodiments, the cultivation infrastructure further comprises a secondary substrate, either introduced, or autologous, to direct cellular growth between the substrates, e.g. to direct attachment, proliferation and hypertrophy of cells on a plane perpendicular to the primary substrate.

In some embodiments, the cultivation infrastructure comprises a hydrogel, a liquid cell culture media, or soft agar.

In some embodiments, the cultivation infrastructure does not comprise a substrate to which cells can adhere. In some embodiments, the cultivation infrastructure comprises a suspension culture, e.g. supporting the growth of a self-adhering biomass, or single-cell suspension in a liquid medium.

In some embodiments, the cultivation infrastructure comprises adherent cells (i.e. those cells that adhere to a substrate). In some embodiments, the cultivation infrastructure comprises non-adherent cells (i.e. those cells that do not adhere to a substrate). In some embodiments, the cultivation infrastructure comprises both adherent and non-adherent cells.

### 6.11. Immortalization

In some embodiments, the method provided herein include a cell line immortalized prior, contemporaneously therewith, or after introducing into the cell any of the polynucleotides described herein.

In some embodiments, immortalization comprises transforming a cell with a telomerase reverse transcriptase (TERT) gene. As used herein, “TERT” refers to telomerase reverse transcriptase (TERT) gene or TERT polypeptide that is a ribonucleoprotein polymerase that maintains telomere ends by addition of the telomere repeat TTAGGG. Telomerase expression plays a role in cellular senescence, as it is normally repressed in postnatal somatic cells resulting in progressive shortening of telomeres. In some embodiments, cells ectopically express the TERT polynucleotide. In some embodiments, the cells are genetically modified and carry stable integrations of one or more copies of the TERT polynucleotide. Exemplary methods for immortalizing a cell line are as described in WO2019014652A1, which is herein incorporated by reference in its entirety.

In some embodiments, increased expression of TERT may be achieved using different approaches. In some embodiments, increased expression of TERT may be achieved by ectopically expressing TERT. In some embodiments, increased expression of TERT may be achieved by introducing targeted mutations in the TERT promoter. In some embodiments, increased expression of TERT may be achieved by activating endogenous TERT expression by an engineered transcriptional activator. In some embodiments, increased expression of TERT may be achieved by transiently transfecting TERT mRNA.

The polynucleotide encoding TERT can be from any organism. The TERT polynucleotide can be from bacteria, plants, fungi, and archaea. The TERT polynucleotide can be from any animal, such as vertebrate and invertebrate animal species. The TERT polynucleotide can be from any vertebrate animal species such as mammals, reptiles, birds, amphibians, and the like. The TERT polynucleotide can be from any mammalian species, such as a human, murine, bovine, porcine, and the like.

In some embodiments, immortalization comprises transforming a cell with a polynucleotide encoding a cyclin-dependent kinase 4 (“CDK4”) protein. In some embodiments, immortalization comprises inactivating a gene encoding an inhibitor of cyclin-dependent kinase 4 (CDK4). Exemplary methods for immortalizing a cell line are as described in WO2017124100A1, which is herein incorporated by reference in its entirety.

### 6.12. Nucleic Acids/Vectors

Also provided herein are polynucleotides comprising coding sequences of any of the growth factor ligands described herein, any of the growth factor receptors described herein, any of the accessory proteins described herein, or a combination thereof.

Also provided herein is a construct (i.e., a vector) that includes any of the polynucleotides described herein. In some embodiments, any of the vectors described herein can be an expression vector. In some embodiments, an expression vector can include one or more promoter sequences (e.g., any of the promoter sequences described herein) operably linked to a coding sequence of any of the growth factor ligands described herein, any of the growth factor receptors described herein, any of the accessory proteins described herein, or a combination thereof. Non-limiting examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway® vectors. In some embodiments, a vector includes sufficient cis-acting elements that supplement expression where the remaining elements needed for expression can be supplied by the host cell (e.g., the cell line).

In some embodiments, a vector includes a polynucleotide comprising a coding sequence of a single growth factor ligand or fragment thereof. In some embodiments, a vector includes a polynucleotide comprising a first coding sequence of a first growth factor ligand and a second coding sequence of a second growth factor ligand. In some embodiments, a vector (e.g., a construct) includes a polynucleotide comprising a first coding sequence of a first growth factor ligand, a second coding sequence of a second growth factor ligand, and a third coding sequence of a third growth factor ligand. In such embodiments where the construct includes coding sequences for two or more growth factor ligands, each of the two or more coding sequences are operably linked to a promoter sequence or to another coding sequence via a self-cleaving polypeptide or IRES. As used herein, the term “operably linked” is well known in the art and refers to genetic components that are combined such that they carry out their normal functions. For example, a coding sequence is operably linked to a promoter when its transcription is under the control of the promoter. In another example, a coding sequence can be operably linked to other coding sequences by a self-cleaving 2A polypeptide or an internal ribosome entry site (IRES). In such cases, the self-cleaving 2A polypeptide allows the second coding sequence to be under the control of the promoter operably linked to the first coding sequence. In some cases, the coding sequences described herein can be operably linked to any other coding sequence described herein using a self-cleaving 2A polypeptide or IRES.

In some embodiments, a vector includes a polynucleotide comprising a coding sequence of a single growth factor receptor or fragment thereof. In some embodiments, a vector includes a polynucleotide comprising a first coding sequence of a first growth factor receptor and a second coding sequence of a second growth factor receptor. In some embodiments, a vector (e.g., a construct) includes a polynucleotide comprising a first coding sequence of a first growth factor receptor, a second coding sequence of a second growth factor receptor, and a third coding sequence of a third growth factor receptor. In such embodiments where the construct includes coding sequences for two or more growth factor receptors, each of the two or more coding sequences are operably linked to a promoter sequence or to another coding sequence via a self-cleaving polypeptide or IRES. In another example, a coding sequence can be operably linked to other coding sequences by a self-cleaving 2A polypeptide or an internal ribosome entry site (IRES). In such cases, the self-cleaving 2A polypeptide allows the second coding sequence to be under the control of the promoter operably linked to the first coding sequence. In some cases, the coding sequences described herein can be operably linked to any other coding sequence described herein using a self-cleaving 2A polypeptide or IRES.

In some embodiments, a single construct comprises a coding sequence of FGF2 or a fragment thereof, and a coding sequence of any of the FGFR described herein or a fragment thereof, and includes a self-cleaving 2A polypeptide or an IRES to operably link the coding sequences. In some embodiments, a single construct comprises a coding sequence of IGF1 or a fragment thereof, and a coding sequence of IGF1R or a fragment thereof, and includes a self-cleaving 2A polypeptide or an IRES to operably link the coding sequences. In some embodiments, a single construct comprises a coding sequence of PDGF or a fragment thereof, and a coding sequence of PDGFR or a fragment thereof, and includes a self-cleaving 2A polypeptide or an IRES to operably link the coding sequences.

Also provided herein are a set of vectors that include two or more vectors. For example, the set of vectors include a first vector comprising a coding sequence of FGF2 or a fragment thereof, and a second vector comprising a coding sequence of FGFR (e.g., any of the FGFRs described herein) or a fragment thereof. In another example, the set of vectors include a first vector comprising a coding sequence of IGF1 or a fragment thereof, and a second vector comprising a coding sequence of IGF1R or a fragment thereof. In yet another example, the set of vectors include a first vector comprising a coding sequence of PDGF (e.g., PDGFB) or a fragment thereof, and a second vector comprising a coding sequence of IGF-1R or a fragment thereof.

In some embodiments, a coding sequence of any one or more of the growth factor ligands described herein, any one or more of the growth factor ligands described herein, any of the accessory protein described herein, or a combination thereof, is operably linked to a promoter. In some embodiments, the promoter is a tissue-specific promoter. In some embodiments, the tissue-specific promoter is a muscle-specific promoter. In some embodiments, the muscle-specific promoter is selected from the group consisting of: skeletal β-action, myosin light chain 2a, dystrophin, SPc-512, muscle creatine kinase, and synthetic muscle promoters. In some embodiments, the promoter is a constitutively active promoter. In some embodiments, the promoter is selected from the group consisting of: EF1 (e.g., EF1alpha), PGK, CMV, RSV, and β-actin. In some embodiments, the promoter is a EF1 (e.g., EF1alpha) promoter. In some embodiments, the promoter is a PGK promoter. In some embodiments, the vector comprises a selectable marker (e.g., puromycin).

In some embodiments, a vector comprises a polynucleotide comprising a first coding sequence (e.g., a coding sequence of any of the growth factor ligands, growth factor receptors, or accessory proteins described herein) operably linked to a first promoter and a polynucleotide comprising a second coding sequence (e.g., a coding sequence of any of the growth factor ligands, growth factor receptors, or accessory proteins described herein) operably linked to a second promoter.

In some embodiments, a vector system is used to integrate a polynucleotide comprising a coding sequence of any one or more of the growth factor ligands described herein, any one or more of the growth factor ligands described herein, any of the accessory protein described herein, or a combination thereof, into the genome of a cell line (e.g., any of the cell lines described herein). In some embodiments, the vector system used for integration is a vector phiC31 Integrase Vector System. Additional non-limiting examples of vectors systems that can be used to integrate a coding sequence of any one or more of the growth factor ligands described herein, any one or more of the growth factor ligands described herein, any of the accessory protein described herein, or a combination thereof, into the genome of a cell line (e.g., any of the cell lines described herein) include: a sleeping beauty transposon system (as described in U.S. Pat. No. 7,985,739), a piggyBac transposition system (as described in US20090042297), CRISPR/Cas-mediated knockin, TALEN-mediated knockin, and viral vector-mediated integration. In such embodiments where integration is mediated via a viral vector, non-limiting examples of viral vectors include adenovirus, adeno-associated virus, lentivirus, retrovirus (e.g., a gamma-retrovirus), or sendai virus.

SEQ ID NOs: 120-122 represent exemplary vector backbones used in this study: Sequences listed in SEQ ID NO: 61-119 were cloned into any one of vectors in SEQ ID NOs: 120-122 using EcoRI and KpnI restriction sites with a kozak sequence GCCACC inserted ahead of ATG start site. FC550A-eBFP2 was created by replacing mRuby sequence in FC550A-empty vector (EVmRuby) with eBFP2. In some embodiments, the polynucleotide sequences includes a sequence having at least 80% (e.g., at least 85%, 90%, 95%, 96%, 97%, 98%, or 99%) sequence identity to a sequence selected from SEQ ID NOs: 61-119.

### 6.13. Methods of Transducing Cells

Methods of introducing nucleic acids and expression vectors into a cell (e.g., an immortalized cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalefection, hydrodynamic delivery, magnetofection, viral transduction (e.g., adenoviral, retroviral, and lentiviral transduction), lipid nanoparticle (LNP) transfection, and nanoparticle transfection.

### 6.14. Kits

Also provided herein are kits comprising any of the cell lines, any of the cells derived from the cell lines, any of the polynucleotides described herein (e.g., any of the coding sequence of any one or more of the growth factor ligands described herein, any one or more of the growth factor ligands described herein, any of the accessory protein described herein, or a combination thereof). In some embodiments, the kit includes instructions for performing any of the methods described herein.

### 6.15. Cells/Cell Lines

Also provided herein are cell line(s) for cultured food production. In some embodiments, the cell line(s) are capable of self-renewal. In some embodiments, the cell line(s) are immortalized cell line(s). In some embodiments, the cell lines are then differentiated to cell types of interest (e.g., myogenic cells).

Also provided herein are immortalized cells (e.g., any of the immortalized cells described herein). In some embodiments, the immortalized cells are fibroblasts. In some embodiments, the immortalized cells comprise any of the nucleic acids described herein that encode any of the myogenic regulatory factors described herein. In some embodiments, an immortalized cell is immortalized prior to performing the methods described herein. In some embodiments, the methods provided herein include a step of immortalizing a cell. In some embodiments, a cell is immortalized by transforming the cell with TERT.

Also provided herein are cells comprising any of the polynucleotides described herein that include any of the growth factor ligands described herein, any of the growth factor receptors described herein, any of the accessory proteins described herein, or a combination thereof.

Also provided herein are cells derived from the cell line(s). Non-limiting examples of cells derived from the immortalized cells (e.g., using the methods described herein) include myoblasts, myotubes, multinucleated myotubes, satellite cells, skeletal muscle fibers, or any combination thereof.

In some embodiments, the cell line is from a livestock, poultry, game or aquatic animal species. In some embodiments, the cell line or immortalized cell line are from a chicken, duck, or turkey. In some embodiments, the cell line or immortalized cell line are from a fish. In some embodiments, the cell line or immortalized cell line are from a livestock species. In some embodiments, the livestock species is porcine or bovine.

In some embodiments, the cell line is selected from any metazoan species. In some embodiments, the cell line is from any animal, such as vertebrate and invertebrate animal species. In some embodiments, the cell line is from any vertebrate animal species such as mammals, reptiles, birds, amphibians, and the like. In some embodiments, the cell line is from any mammalian species such as a human, murine, bovine, porcine, poultry, and the like. In some embodiments, the cell line is derived from a species selected from including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus.

In some embodiments, the cell line (e.g., a cell line that is ultimately immortalized) is isolated from Gallus gallus (chicken). In some embodiments, the cell is isolated from chicken skin. In some embodiments, the cell is isolated from chicken muscle. In some embodiment, the cell is isolated from a chicken (e.g., chicken skin or chicken muscle) and cultured until a monoculture of cells is established (e.g., a monoculture of fibroblasts originating from the isolated chicken cells).

In some embodiments, the cell line (e.g., a cell line that is ultimately immortalized) is selected from the group consisting of: a myoblast, an immortalized myoblast, an immortalized primary myoblast, a muscle satellite cell, and a muscle stem cell. In some embodiments, the immortalized cell is an immortalized myoblast or an immortalized primary myoblast.

In some embodiments, the cell line (e.g., a cell line that is ultimately immortalized) is a fibroblast. For example, the cell is an immortalized fibroblast.

In some embodiments, skeletal muscle satellite cells are isolated from a chicken. In adults these are quiescent mononucleated myogenic cells that act as a reserve population of cells, able to proliferate and/or differentiate upon stimulation and give rise to regenerated muscle and to more satellite cells.

In some embodiments, an immortalized cell is not a stem cell (e.g., a muscle stem cell or a muscle satellite cell). In some embodiments, an immortalized cell is not a pluripotent stem cell (e.g., an embryonic stem cell or an induced pluripotent stem cell).

Also provided herein are cell banks comprising immortalized cell lines (e.g., immortalized fibroblast cells lines) generated according to the methods described herein.

Also provided herein is cell-based meat suitable for consumption.

### 7. ADDITIONAL EMBODIMENTS

Embodiment 1. A method of engineering a cell line for reduced dependence on exogenous growth factors, comprising: (a) introducing into the cell line one or more of: (i) a polynucleotide comprising a coding sequence of a growth factor ligand; (ii) a polynucleotide comprising a coding sequence of a growth factor receptor; or (iii) a polynucleotide comprising a coding sequence of an activated downstream growth factor target; and (b) culturing the cell line in a cultivation infrastructure.

Embodiment 2. A method of increasing the concentration of a growth factor ligand in culture medium of cells in culture, comprising: (a) introducing into a cell line one or more of: (i) a polynucleotide comprising a coding sequence of a growth factor ligand; and (ii) a polynucleotide comprising a coding sequence of a growth factor receptor; or (iii) a polynucleotide comprising a coding sequence of an activated downstream growth factor target; and (b) culturing the cell line in a cultivation infrastructure.

Embodiment 3. A method for improving anchorage independent growth in a cell line, comprising: (a) introducing into the cell line one or more of: (i) a polynucleotide comprising a coding sequence of a growth factor ligand; and (ii) a polynucleotide comprising a coding sequence of a growth factor receptor; or (iii) a polynucleotide comprising a coding sequence of an activated downstream growth factor target; and (b) culturing the cell line in a cultivation infrastructure.

Embodiment 4. A method for increasing the cell density of a culture comprising a cell line, comprising: (a) introducing into the cell line one or more of: (i) a polynucleotide comprising a coding sequence of a growth factor ligand; and (ii) a polynucleotide comprising a coding sequence of a growth factor receptor; or (iii) a polynucleotide comprising a coding sequence of an activated growth factor receptor; and (b) culturing the cell line in a cultivation infrastructure.

Embodiment 5. The method of any one of embodiments 1-4, wherein the growth factor ligand is selected from basic fibroblast growth factor (FGF2), insulin-like growth factor 1 (IGF1), and platelet-derived growth factor subunit B (PDGFb).

Embodiment 6. The method of any one of embodiments 1-5, wherein the polynucleotide comprising a coding sequence of a growth factor ligand further comprises an additional two or more growth factor ligands, wherein each of the additional growth factor ligands is selected from FGF2, IGF1, and PDGFb.

Embodiment 7. The method of embodiment 5 or 6, wherein the growth factor ligand is FGF2.

Embodiment 8. The method of embodiment 7, wherein the FGF2 comprises an amino acid sequence having at least 80% sequence identity to a sequence selected from SEQ ID NO: 1-15.

Embodiment 9. The method of embodiment 5, wherein the growth factor ligand is IGF1.

Embodiment 10. The method of embodiment 9, wherein the IGF1 comprises an amino acid sequence having at least 80% sequence identity to a sequence selected from SEQ ID NO: 16-17.

Embodiment 11. The method of embodiment 5 or 6, wherein the growth factor ligand is PDGFb.

Embodiment 12. The method of embodiment 11, wherein the PDGFb comprises an amino acid sequence having at least 80% sequence identity to a sequence selected from SEQ ID NO: 18-21.

Embodiment 13. The method of any one of embodiments 1-12, wherein the polynucleotide comprising a coding sequence of a growth factor ligand further comprises a coding sequence of a signal peptide located 5′ to the coding sequence of the growth factor ligand, and wherein the signal sequence and the growth factor ligand are a fusion protein.

Embodiment 14. The method of embodiment 13, wherein the signal sequence comprises an amino acid sequence having at least 80% sequence identity to a sequence selected from SEQ ID NO: 22-31.

Embodiment 15. The method of any one of embodiments 1-14, wherein the polynucleotide comprising a coding sequence of a growth factor ligand further comprises a regulatory sequence operably linked to the coding sequence of the growth factor ligand and/or additional growth factor ligands.

Embodiment 16. The method of embodiment 15, wherein the regulatory sequence comprises a promoter selected from: an inducible promoter, a tissue specific promoter, and a constitutively active promoter.

Embodiment 17. The method of embodiment 15 or 16, wherein the promoter is selected from EF1alpha, PGK, CMV, RSV, and β-actin.

Embodiment 18. The method of any one of embodiments 1-17, wherein the polynucleotide comprising a coding sequence of a growth factor ligand comprises a sequence encoding a polycistronic mRNA, wherein the polycistronic mRNA comprises the coding sequence of the growth factor ligand and the coding sequence one or more additional growth factor ligands.

Embodiment 19. The method of embodiment 18, wherein the polynucleotide comprising a coding sequence of a growth factor ligand comprises one or more sequences encoding a self-cleaving peptide, one or more internal ribosome entry sites (IRES), or a combination thereof.

Embodiment 20. The method of any one of embodiment 1-19, wherein activity and/or expression of the growth factor ligand is controllable.

Embodiment 21. The method of embodiment 20, where activity and/or expression of the growth factor ligand is controlled using an inducible promoter, an inducible tag, or a degradation tag.

Embodiment 22. The method of embodiment 21, wherein expression of the growth factor ligands is controlled using an inducible promoter.

Embodiment 23. The method of embodiment 22, wherein the coding sequence of the growth factor ligand is operably linked to the inducible promoter.

Embodiment 24. The method of embodiment 22 or 23, further comprising:

maintaining the engineered cell line in a culture medium comprising a molecule capable of controlling the inducible promoter, thereby enabling expression of the growth factor ligand.

Embodiment 25. The method of embodiment 21, wherein activity of the growth factor ligand is controlled using an inducible tag.

Embodiment 26. The method of embodiment 25, wherein the polynucleotide comprising a coding sequence of a growth factor ligand further comprises a coding sequence of an inducible tag located 5′ or 3′ to the coding sequence of the growth factor ligand, and wherein the inducible tag and the growth factor ligand are a fusion protein.

Embodiment 27. The method of embodiment 25 or 26, further comprising: maintaining the engineered cell line in a culture medium comprising a molecule capable of controlling the inducible tag, thereby inducing activity of the growth factor ligand/fusion protein.

Embodiment 28. The method of embodiment 21, wherein activity of the one or more growth factor ligands is controlled using a degradation tag.

Embodiment 29. The method of any one of embodiments 28, wherein the polynucleotide comprising a coding sequence of a growth factor ligand further comprises a coding sequence of a degradation tag located 5′ or 3′ to the coding sequence of the growth factor ligand, and wherein the degradation tag and the growth factor ligand are a fusion protein.

Embodiment 30. The method of embodiment 28 or 29, further comprising: maintaining the engineered cell line in a culture medium comprising a molecule capable of controlling the degradation tag, thereby targeting the growth factor ligand/fusion protein for degradation.

Embodiment 31. The method of any one of embodiments 1-30, wherein (a) the concentration of FGF2 in the culture medium is increased by at least 0.001 ng/mL as compared to cell lines not engineered to include a polynucleotide encoding FGF2; (b) the concentration of IGF-1 in the culture medium is increased by at least 0.001 ng/mL as compared to cell lines not engineered to include a polynucleotide encoding IGF1; and/or (c) the concentration of PDGFb in the culture medium is increased by at least 0.001 ng/mL as compared to cell lines not engineered to include a polynucleotide encoding PDGFb.

Embodiment 32. The method of any one of embodiments 1-30, wherein (a) the concentration of FGF2 in the culture medium is increased by at least 2.5% as compared to cell lines not engineered to include a polynucleotide encoding FGF2; (b) the concentration of IGF-1 in the culture medium is increased by at least 2.5% as compared to cell lines not engineered to include a polynucleotide encoding IGF1; and/or (c) the concentration of PDGFb in the culture medium is increased by at least 2.5% as compared to cell lines not engineered to include a polynucleotide encoding PDGFb.

Embodiment 33. The method of any one of embodiments 1-32, wherein the growth factor receptor is selected from fibroblast growth factor receptor (FGFR), insulin growth factor 1 receptor (IGF1R), and platelet-derived growth factor receptor (PDGFR).

Embodiment 34. The method of embodiment 33, wherein the polynucleotide comprising a coding sequence of a growth factor receptor further comprises an additional two or more growth factor receptors, wherein each additional growth factor receptor is selected from FGFR, IGFR, and PDGFR.

Embodiment 35. The method of embodiment 33 or 34, wherein the growth factor receptor is an FGFR selected from FGFR1, FGFR2, FGFR3, and FGFR4.

Embodiment 36. The method of embodiment 35, wherein the FGFR comprises an amino acid sequence having at least 80% sequence identity to a sequence selected from SEQ ID NO: 32-49.

Embodiment 37. The method of embodiment 33 or 34, wherein the growth factor receptor is IGF1R.

Embodiment 38. The method of embodiment 37, wherein the IGF1R comprises an amino acid sequence having at least 80% sequence identity to a sequence selected from SEQ ID NO: 50-51.

Embodiment 39. The method of embodiment 33 or 34, wherein the growth factor receptor is PDGFR.

Embodiment 40. The method of embodiment 39, wherein the PDGFR comprises an amino acid sequence having at least 80% sequence identity to a sequence selected from SEQ ID NO: 52-58.

Embodiment 41. The method of any one of embodiments 1-40, wherein the polynucleotide comprising a coding sequence of an activated downstream growth factor target comprises a growth factor receptor comprising one or more amino acid insertions, deletions, or substitutions that result in the receptor being constitutively activated.

Embodiment 42. The method of any one of embodiments 33-41, wherein the polynucleotide comprising a coding sequence of a growth factor receptor comprises a regulatory sequence operably linked to the coding sequence of the growth factor receptor and/or the coding sequence of the additional growth factor receptors.

Embodiment 43. The method of embodiment 42, wherein the regulatory sequence comprises a promoter selected from: an inducible promoter, a tissue specific promoter, and a constitutively active promoter.

Embodiment 44. The method of embodiment 42 or 43, wherein the promoter is selected from EF1alpha, PGK, CMV, RSV, and β-actin.

Embodiment 45. The method of any one of embodiments 1-44, wherein the polynucleotide comprising a coding sequence of a growth factor receptor comprises a sequence encoding a polycistronic mRNA, wherein the polycistronic mRNA comprises the coding sequence of the growth factor receptor and the coding sequence of the two or more additional growth factor receptors.

Embodiment 46. The method of embodiment 45, wherein the polynucleotide comprising a coding sequence of a growth factor receptor comprises one or more sequences encoding a self-cleaving peptide, one or more internal ribosome entry sites (IRES), or a combination thereof.

Embodiment 47. The method of any one of embodiment 1-46, wherein activity and/or expression of the growth factor receptors is controllable.

Embodiment 48. The method of embodiment 47, wherein expression of the growth factor receptor is controlled using an inducible promoter, an inducible tag, and a degradation tag.

Embodiment 49. The method of embodiment 48, wherein expression of the growth factor receptor is controlled using an inducible promoter.

Embodiment 50. The method of embodiment 49, wherein the coding sequence of the growth factor receptor is operably linked to the inducible promoter.

Embodiment 51. The method of embodiment 49 or 50, further comprising: maintaining the engineered cell line in a culture medium comprising a molecule capable of controlling the inducible promoter, thereby enabling expression of the growth factor receptor.

Embodiment 52. The method of embodiment 48, wherein activity of the growth factor receptor is controlled using an inducible tag.

Embodiment 53. The method of embodiment 52, wherein the polynucleotide comprising a coding sequence of a growth factor receptor further comprises a coding sequence of an inducible tag located 5′ or 3′ to the coding sequence of the growth factor receptor, and wherein the inducible tag and the growth factor receptor are a fusion protein.

Embodiment 54. The method of embodiment 52 or 53, further comprising: maintaining the engineered cell line in a culture medium comprising a molecule capable of controlling the inducible tag, thereby inducing activity of the growth factor receptor/fusion protein.

Embodiment 55. The method of embodiment 48, wherein activity of the one or more growth factor ligands is controlled using a degradation tag.

Embodiment 56. The method of embodiment 55, wherein the polynucleotide comprising a coding sequence of a growth factor receptor further comprises a coding sequence of a degradation tag located 5′ or 3′ to the coding sequence of the growth factor receptor, and wherein the degradation tag and the growth factor receptor are a fusion protein.

Embodiment 57. The method of embodiment 55 or 56, further comprising: maintaining the engineered cell line in a culture medium comprising a molecule capable of controlling the degradation tag, thereby targeting the growth factor receptor/fusion protein for degradation.

Embodiment 58. The method of any one of embodiments 1-57, further comprising introducing a polynucleotide sequence encoding an accessory protein.

Embodiment 59. The method of any one of embodiments 1-58, wherein the polynucleotide comprising a coding sequence of a growth factor ligand and/or the polynucleotide comprising a coding sequence of a growth factor receptor further comprises a coding sequence of an accessory protein.

Embodiment 60. The method of embodiment 58 or 59, wherein the accessory protein is fibroblast growth factor binding protein (FGFBP).

Embodiment 61. The method of embodiment 60, wherein the FGFBP comprises an amino acid sequence having at least 80% sequence identity to a sequence of SEQ ID NO: 59.

Embodiment 62. The method of embodiment 58 or 59, wherein the accessory protein is RASV12.

Embodiment 63. The method of embodiment 62, wherein the RASV12 comprises an amino acid sequence having at least 80% sequence identity to a sequence of SEQ ID NO: 60.

Embodiment 64. The method of any one of embodiments 1-63, wherein the method comprises introducing into the cell: (i) a polynucleotide comprising a coding sequence of FGF2, and a polynucleotide comprising a FGFR; (ii) a polynucleotide comprising a coding sequence of IGF1, and a polynucleotide comprising an IGF1R; (iii) a polynucleotide comprising a coding sequence of PDGF, and a polynucleotide comprising a PDGFR, or (iv) a combination selected from: (i) and (ii), (ii) and (iii), (ii) and iii), and (i), (ii), and (iii).

Embodiment 65. The method of any one of embodiments 1-64, wherein the cell line is from a livestock, poultry, game or aquatic animal species.

Embodiment 66. The method of embodiment 65, wherein the cell line is from a chicken, duck, or turkey.

Embodiment 67. The method of embodiment 65, wherein the cell line is from a fish.

Embodiment 68. The method of embodiment 65, wherein the cell line is from a livestock species.

Embodiment 69. The method of embodiment 65, wherein the livestock species is porcine or bovine.

Embodiment 70. The method of any one of embodiments 1-64, wherein the cells are from any animal species intended for human or non-human dietary consumption.

Embodiment 71. The method of any one of embodiments 1-70, wherein the cells are myogenic cells.

Embodiment 72. The method of embodiment 71, wherein the myogenic cells are myoblasts, myocytes, satellite cells, side population cells, muscle derived stem cells, mesenchymal stem cells, myogenic pericytes, or mesoangioblasts.

Embodiment 73. The method of any one of embodiments 1-72, wherein the cells are non-myogenic cells.

Embodiment 74. An in vitro method for producing cell-based meat suitable for consumption, comprising: (a) introducing into a cell one or more of: (i) a polynucleotide comprising a coding sequence of a growth factor; (ii) a polynucleotide comprising a coding sequence of a growth factor receptor; or (iii) a polynucleotide comprising a coding sequence of an activated downstream growth factor target; and (b) inducing myogenic specific differentiation, wherein the differentiated cells form myocytes and multinucleated myotubes; (c) culturing the myocytes and myotubes to generate skeletal muscle fibers, thereby producing a cell-based meat suitable for consumption.

Embodiment 75. The method of embodiment 74, wherein the cell line is from a livestock, poultry, game or aquatic animal species.

Embodiment 76. The method of embodiment 75, wherein the cell line is from a chicken, duck, or turkey.

Embodiment 77. The method of embodiment 75, wherein the cell line is from a fish.

Embodiment 78. The method of embodiment 75, wherein the cell line is from a livestock species.

Embodiment 79. The method of embodiment 75, wherein the livestock species is porcine or bovine.

Embodiment 80. The method of embodiment 74, wherein the cells are from any animal species intended for human or non-human dietary consumption.

Embodiment 81. The method of any one of embodiments 74-80, wherein the cells are myogenic cells.

Embodiment 82. The method of embodiment 81, wherein the myogenic cells are myoblasts, myocytes, satellite cells, side population cells, muscle derived stem cells, mesenchymal stem cells, myogenic pericytes, or mesoangioblasts.

Embodiment 83. The method of any one of embodiments 74-82, wherein the cells are non-myogenic cells.

Embodiment 84. The method of any one of embodiments 74-83, wherein the growth factor ligand is selected from basic fibroblast growth factor (FGF2), insulin-like growth factor 1 (IGF1), and platelet-derived growth factor subunit B (PDGFb).

Embodiment 85. The method of embodiment 84, wherein the polynucleotide comprising a coding sequence of a growth factor ligand further comprises an additional two or more growth factor ligands, wherein each of the additional growth factor ligands is selected from FGF2, IGF1, and PDGFb.

Embodiment 86. The method of embodiment 84 or 85, wherein the growth factor ligands is FGF2.

Embodiment 87. The method of embodiment 86, wherein the FGF2 comprises an amino acid sequence having at least 80% sequence identity to a sequence selected from SEQ ID NO: 1-15.

Embodiment 88. The method of embodiment 84 or 85, wherein the growth factor ligands is IGF1.

Embodiment 89. The method of embodiment 88, wherein the IGF1 comprises an amino acid sequence having at least 80% sequence identity to a sequence selected from SEQ ID NO: 16-17.

Embodiment 90. The method of embodiment 84 or 85, wherein the growth factor ligands is PDGFb.

Embodiment 91. The method of embodiment 90, wherein the PDGFb comprises an amino acid sequence having at least 80% sequence identity to a sequence selected from SEQ ID NO: 18-21.

Embodiment 92. The method of any one of embodiments 74-91, wherein the polynucleotide comprising a coding sequence of a growth factor ligand further comprises a coding sequence of a signal peptide located 5′ to the coding sequence of the growth factor ligand, and wherein the signal sequence and the growth factor ligand are a fusion protein.

Embodiment 93. The method of embodiment 92, wherein the signal sequence comprises an amino acid sequence having at least 80% sequence identity to a sequence selected from SEQ ID NO: 22-31.

Embodiment 94. The method of any one of embodiments 74-93, wherein the polynucleotide comprising a coding sequence of a growth factor ligand comprises a regulatory sequence operably linked to the coding sequence of the growth factor ligand and/or additional growth factor ligands.

Embodiment 95. The method of embodiment 94, wherein the regulatory sequence comprises a promoter selected from: an inducible promoter, a tissue specific promoter, and a constitutively active promoter.

Embodiment 96. The method of embodiment 94 or 95, wherein the promoter is selected from EF1alpha, PGK, CMV, RSV, and β-actin.

Embodiment 97. The method of any one of embodiments 74-96, wherein the polynucleotide comprising a coding sequence of a growth factor ligand comprises a sequence encoding a polycistronic mRNA, wherein the polycistronic mRNA comprises the coding sequence of the growth factor ligand and the coding sequence one or more additional growth factor ligands.

Embodiment 98. The method of embodiment 97, wherein the polynucleotide comprising a coding sequence of a growth factor ligand further comprises one or more sequences encoding a self-cleaving peptide, one or more internal ribosome entry sites (IRES), or a combination thereof.

Embodiment 99. The method of any one of embodiment 74-99, wherein activity and/or expression of the growth factor ligand is controllable.

Embodiment 100. The method of embodiment 99, wherein activity and/or expression of the growth factor ligand is controlled using an inducible promoter, an inducible tag, or a degradation tag.

Embodiment 101. The method of embodiment 100, wherein expression of the growth factor ligands is controlled using an inducible promoter.

Embodiment 102. The method of embodiment 101, wherein the coding sequence of the growth factor ligand is operably linked to the inducible promoter.

Embodiment 103. The method of embodiment 101 or 102, further comprising: maintaining the engineered cell line in a culture medium comprising a molecule capable of controlling the inducible promoter, thereby enabling expression of the growth factor ligand.

Embodiment 104. The method of embodiment 100, wherein activity of the growth factor ligand is controlled using an inducible tag.

Embodiment 105. The method of embodiment 104, wherein the polynucleotide comprising a coding sequence of a growth factor ligand further comprises a coding sequence of an inducible tag located 5′ or 3′ to the coding sequence of the growth factor ligand, and wherein the inducible tag and the growth factor ligand are a fusion protein.

Embodiment 106. The method of embodiment 104 or 105, further comprising: maintaining the engineered cell line in a culture medium comprising a molecule capable of controlling the inducible tag, thereby inducing activity of the growth factor ligand/fusion protein.

Embodiment 107. The method of embodiment 100, wherein activity of the one or more growth factor ligands is controlled using a degradation tag.

Embodiment 108. The method of any one of embodiments 107, wherein the polynucleotide comprising a coding sequence of a growth factor ligand further comprises a coding sequence of a degradation tag located 5′ or 3′ to the coding sequence of the growth factor ligand, and wherein the degradation tag and the growth factor ligand are a fusion protein.

Embodiment 109. The method of embodiment 107 or 108, further comprising: maintaining the engineered cell line in a culture medium comprising a molecule capable of controlling the degradation tag, thereby targeting the growth factor ligand/fusion protein for degradation.

Embodiment 110. The method of any one of embodiments 74-109, wherein the growth factor receptor is selected from fibroblast growth factor receptor (FGFR), insulin growth factor 1 receptor (IGF1R), and platelet-derived growth factor receptor (PDGFR).

Embodiment 111. The method of embodiment 110, wherein the polynucleotide comprising a coding sequence of a growth factor receptor further comprises an additional two or more growth factor receptors, wherein each additional growth factor receptor is selected from FGFR, IGFR, and PDGFR.

Embodiment 112. The method of embodiment 110 or 111, wherein the growth factor receptor is an FGFR selected from FGFR1, FGFR2, FGFR3, and FGFR4.

Embodiment 113. The method of embodiment 112, wherein the FGFR comprises an amino acid sequence having at least 80% sequence identity to a sequence selected from SEQ ID NO: 32-49.

Embodiment 114. The method of embodiment 110 or 111, wherein the growth factor receptor is IGF1R.

Embodiment 115. The method of embodiment 114, wherein the IGF1R comprises an amino acid sequence having at least 80% sequence identity to a sequence selected from SEQ ID NO: 50-51.

Embodiment 116. The method of embodiment 110 or 111, wherein the growth factor receptor is PDGFR.

Embodiment 117. The method of embodiment 116, wherein the PDGFR comprises an amino acid sequence having at least 80% sequence identity to a sequence selected from SEQ ID NO: 52-58.

Embodiment 118. The method of any one of embodiments 74-117, wherein the polynucleotide comprising a coding sequence of an activated downstream growth factor target comprises a growth factor receptor comprising one or more amino acid insertions, deletions, or substitutions that result in the receptor being constitutively activated.

Embodiment 119. The method of any one of embodiments 110-118, wherein the polynucleotide comprising a coding sequence of a growth factor receptor comprises a regulatory sequence operably linked to the coding sequence of the growth factor receptor and/or the coding sequence of the additional growth factor receptors.

Embodiment 120. The method of embodiment 119, wherein the regulatory sequence comprises a promoter selected from: an inducible promoter, a tissue specific promoter, and a constitutively active promoter.

Embodiment 121. The method of embodiment 119 or 120, wherein the promoter is selected from EF1alpha, PGK, CMV, RSV, and β-actin.

Embodiment 122. The method of any one of embodiments 74-121, wherein the polynucleotide comprising a coding sequence of a growth factor receptor comprises a sequence encoding a polycistronic mRNA, wherein the polycistronic mRNA comprises the coding sequence of the growth factor receptor and the coding sequence of the two or more additional growth factor receptors.

Embodiment 123. The method of embodiment 122, wherein the polynucleotide comprising a coding sequence of a growth factor receptor comprises one or more sequences encoding a self-cleaving peptide, one or more internal ribosome entry sites (IRES), or both.

Embodiment 124. The method of any one of embodiment 74-123, wherein activity and/or expression of the growth factor receptors is controllable.

Embodiment 125. The method of embodiment 124, where expression of the growth factor receptor is controlled using an inducible promoter, an inducible tag, and a degradation tag.

Embodiment 126. The method of embodiment 125, wherein expression of the growth factor receptor is controlled using an inducible promoter.

Embodiment 127. The method of embodiment 126, wherein the coding sequence of the growth factor receptor is operably linked to the inducible promoter.

Embodiment 128. The method of embodiment 126 or 127, further comprising:

maintaining the engineered cell line in a culture medium comprising a molecule capable of controlling the inducible promoter, thereby enabling expression of the growth factor receptor.

Embodiment 129. The method of embodiment 125, wherein activity of the growth factor receptor is controlled using an inducible tag.

Embodiment 130. The method of embodiment 129, wherein the polynucleotide comprising a coding sequence of a growth factor receptor further comprises a coding sequence of an inducible tag located 5′ or 3′ to the coding sequence of the growth factor receptor, and wherein the inducible tag and the growth factor receptor are a fusion protein.

Embodiment 131. The method of embodiment 129 or 130, further comprising: maintaining the engineered cell line in a culture medium comprising a molecule capable of controlling the inducible tag, thereby inducing activity of the growth factor receptor/fusion protein.

Embodiment 132. The method of embodiment 125, wherein activity of the one or more growth factor ligands is controlled using a degradation tag.

Embodiment 133. The method of embodiment 132, wherein the polynucleotide comprising a coding sequence of a growth factor receptor further comprises a coding sequence of a degradation tag located 5′ or 3′ to the coding sequence of the growth factor receptor, and wherein the degradation tag and the growth factor receptor are a fusion protein.

Embodiment 134. The method of embodiment 132 or 133, further comprising: maintaining the engineered cell line in a culture medium comprising a molecule capable of controlling the degradation tag, thereby targeting the growth factor receptor/fusion protein for degradation.

Embodiment 135. The method of any one of embodiments 74-134, further comprising introducing a polynucleotide sequence encoding an accessory protein.

Embodiment 136. The method of any one of embodiment 74-134, wherein the polynucleotide comprising a coding sequence of a growth factor ligand, the polynucleotide comprising a coding sequence of a growth factor receptor, or both, further comprises a coding sequence of an accessory protein.

Embodiment 137. The method of embodiment 135 or 136, wherein the accessory protein is fibroblast growth factor binding protein (FGFBP).

Embodiment 138. The method of embodiment 137, wherein the FGFBP comprises an amino acid sequence having at least 80% sequence identity to a sequence of SEQ ID NO: 59.

Embodiment 139. The method of embodiment 135 or 136, wherein the accessory protein is RASV12.

Embodiment 140. The method of embodiment 139, wherein the RASV12 comprises an amino acid sequence having at least 80% sequence identity to a sequence of SEQ ID NO: 60.

Embodiment 141. The method of any one of embodiments 74-140, wherein the method comprises introducing into the cell:
(i) a polynucleotide comprising a coding sequence of FGF2, and a polynucleotide comprising a coding sequence of FGFR;
(ii) a polynucleotide comprising a coding sequence of IGF1, and a polynucleotide comprising a coding sequence of IGF1R;
(iii) a polynucleotide comprising a coding sequence of PDGF, and a polynucleotide comprising a coding sequence of PDGFR, or
(iv) a combination selected from: (i) and (ii), (ii) and (iii), (ii) and iii), and (i), (ii), and (iii).

Embodiment 142. A myogenic cell suitable for consumption comprising cells having increased expression of FGF2, IGF1, PDGFb, FGFR, IGF1R, or PDGFR, or a combination thereof.

Embodiment 143. A population of cells suitable for consumption comprising cells having increased expression of FGF2, IGF1, PDGFb, FGFR, IGF1R, or PDGFR, or a combination thereof.

Embodiment 144. A vector comprising a polynucleotide encoding a fusion protein comprising a coding sequence of a signaling sequence and a coding sequence of a growth factor ligand, and optionally a coding sequence of an inducible tag or a degradation tag.

Embodiment 145. A cell comprising a polynucleotide encoding a fusion protein comprising a coding sequence of a signaling sequence and a coding sequence of a growth factor ligand, and optionally a coding sequence of an inducible tag or a degradation tag.

Embodiment 146. A vector comprising a polynucleotide comprising a first coding sequence of a growth factor ligand or a fragment thereof, and a second coding sequence of growth factor receptor or a fragment thereof.

Embodiment 147. A cell comprising a polynucleotide comprising a coding sequence of a growth factor ligand or a fragment thereof, and a polynucleotide comprising a coding sequence of a growth factor receptor or a fragment thereof.

Embodiment 148. A cell-based meat suitable for consumption produced using the methods of any one of embodiments 74-141.

### 8. EXAMPLES

### 8.1. Summary of Experimental Observations

Applicant evaluated cell lines harboring a polynucleotide comprising a coding sequence of FGF2, IGF1, and/or PDGF for cell proliferation, impact on adherence, and amount of FGF2, IGF1, PDGF secreted into the culture medium. Applicant evaluated whether cells engineered to express and secrete FGF2, IGF1, and/or PDGF ligands could be grown in serum-free media (serum is known to contain an assortment of growth factors) in the absence of exogenous growth factors (i.e., media not supplemented with FGF2, IGF1, and/or PDGF). Applicant further evaluated whether cells engineered to express FGF2, IGF1, and/or PDGF receptors could be grown in serum-free media in the absence of exogenous growth factors. Applicant additionally evaluated whether cells engineered with specific promoters to drive expression of the exogenous polynucleotides or signaling peptides fused to the FGF2, IGF1, and/or PDG ligands could be grown in serum-free media in the absence of exogenous growth factors and increase growth factor secretion, respectively. Applicant demonstrated that introducing the polynucleotide comprising a coding sequence of an FGF2, IGF1, and/or PDGF ligand, receptor, or activated receptor into an immortalized cell line and culturing the cell line in serum free media resulted in cell proliferation and packed cell volumes similar to positive control cell lines grown in serum and/or in culture medium supplemented with exogenous growth factors. As a negative control, cell lines grown without being engineered to express an exogenous polynucleotide, without serum, and without exogenous growth factors failed to proliferate over 72 hours and also exhibited decreases in population doubling times. Additionally, Applicant found that specific promoters further improved cell line performance when introducing the polynucleotide comprising a coding sequence of an FGF2, IGF1, and/or PDGF ligand, receptor, or activated receptor into an immortalized cell line and culturing the cell line in serum free media without any addition of exogenous growth factors. In addition, Applicant demonstrated that cell lines harboring the polynucleotide comprising a coding sequence of an FGF2, IGF1, and/or PDGF ligand secreted these growth factors into the serum-free media and this secretion was increased further when a polynucleotide comprising a coding sequence for a signaling peptide was combined with a polynucleotide comprising a coding sequence for growth factor to provide a fusion protein coding sequence. Applicant has further found that species to species and cell to cell variability impacts the efficacy of this approach and of these gene targets. For instance, Applicant found that introduction of polynucleotides comprising coding sequences for FGF2 ligands (of salmon, chicken, and bovine varieties) into bovine cell lines failed to generate a growth factor independent cell line. Application further notes that, even within the same species, cells of a different type often respond differently to any given growth factor and may require different growth factor targets.

Applicant further tested the cell lines harboring a polynucleotide comprising a coding sequence of FGF2 for impact on anchorage-dependent growth. Applicant demonstrated that introducing the polynucleotide comprising a coding sequence of FGF2 into an immortalized cell line resulted in an increase in the number of non-adherent cells capable of anchorage-independent growth.

Applicant also evaluated cell lines harboring a polynucleotide comprising a coding sequence of IGF1 for cell proliferation rate (e.g., viable cell density (VCD)) and packed cell volumes (PCV), where PCV is used as a proxy for cell biomass. In particular, Applicant demonstrated that introducing the polynucleotide comprising a coding sequence of IGF1 into an immortalized cell line and culturing the cell line in serum-free media in the absence of exogenous IGF1 resulted in increased viable cell densities as compared to the controls. Applicant also showed the cells engineered to express IGF1 had greater PCVs as compared to controls.

Applicant also evaluated cell lines harboring a polynucleotide comprising a coding sequence of IGF1 and a polynucleotide encoding an FGF2 for cell proliferation rate (e.g., viable cell density (VCD)) and packed cell volumes (PCV), where PCV is used as a proxy for cell biomass. Applicant demonstrated that introducing the polynucleotide comprising a coding sequence of IGF1 and a polynucleotide sequence encoding an FGF2 into an immortalized cell line and culturing the cell line in serum-free media in the absence of growth factors resulted in increased viable cell densities and PCVs as compared to controls.

Applicant also evaluated cell lines harboring a polynucleotide comprising a coding sequence of IGF1 and a polynucleotide encoding an FGF2 receptor for cell proliferation rate (e.g., viable cell density (VCD)) and packed cell volumes (PCV. Applicant demonstrated that introducing the polynucleotide comprising a coding sequence of IGF1 and a polynucleotide sequence encoding an FGF2 receptor into a cell line and culturing the cell line in serum-free media in the absence of growth factors resulted in increased viable cell densities and PCVs as compared to controls.

Lastly, Applicant also evaluated cell lines harboring a polynucleotide comprising a coding sequence of PDGF or a polynucleotide encoding an PDGF receptor for cell proliferation rate (e.g., viable cell density (VCD)) and packed cell volumes (PCV. Applicant demonstrated that introducing the polynucleotide comprising a coding sequence of PDGF or a polynucleotide sequence encoding an PDGF receptor into a cell line and culturing the cell line in serum-free media in the absence of growth factors resulted in increased viable cell densities and PCVs as compared to controls.

Overall, this work demonstrated the ability to engineer cells to have reduced dependence on exogenous growth factors without compromising the cell's ability to proliferate or differentiate into myogenic cells. In particular, this work demonstrated that engineering cells to express growth factor ligands and/or growth factor receptors results in engineered cells that (i) maintain cell proliferation rates similar to positive controls, (ii) have an increase in the number of non-adherent cells (i.e., cells that exhibit anchorage-independent growth) as compared to controls, and (iii) have an increase in packed cell volumes as compared to both positive and negative controls, all while cells are grown in the absence of one or more exogenous growth factors. These findings are important because manufacturing cells suitable for consumption requires vast amounts of exogenous growth factor ligands in order to sufficiently expand the cells and adapt them to the appropriate culture formats (e.g., suspension culture). The engineered cells provided herein supply their own source of growth factor signaling-thereby bypassing or at least reducing the need to supplement the cultures with exogenous growth factors. Moreover, by promoting anchorage independent growth, which is currently essential for manufacturing cell-based meats suitable for consumption, the engineered cell lines provided herein increase the efficiency by which cell based meats suitable for consumption can be produced.

### 8.2. Methods

### 8.2.1. Cell Line Production.

In order to generate cell lines with integrated polynucleotides, a PhiC31 Integrase Expression Plasmid system was used (System Biosciences). Briefly, coding sequences of genes of interest (e.g., coding sequence of FGF2 or coding sequence of IGF1) were cloned into a PhiC31 dual promoter expression plasmid (System Biosciences, Cat No. FC550A-1). Cell lines were transfected with the plasmid containing the coding sequences of the gene(s) of interest (e.g., FGF2 or IGF1) and a plasmid containing an integrase (PhiC31 integrase, System Biosciences, Cat No. FC200PA-1) to integrate the coding sequences into the genome. In some cases, the Phi31 plasmids also included a red fluorescent protein as a reporter. Cell lines with integrated plasmids were selected using puromycin and assessed for transgene expression and/or fluorescent marker expression. Cell lines exhibiting stable expression were selected for further analysis.

### 8.2.2. Assessment of Secreted FGF2 or Secreted IGF1

Engineered cells were serum starved and plated into serum free media (no FGF2) for 72 hours. FGF2 ELISA (DFB50 (R&D Biosystems Quantikine Human FGF basic Immunoassay kit) was used to determine the concentration of FGF2 in the supernatant.

Engineered cells were serum starved and plated into serum free media (no IGF1) for 72 hours. IGF1 ELISA ((R&D Biosystems) was used to determine the concentration of IGF1 in the supernatant.

### 8.2.3. Assessment of Cell Proliferation

A colorimetric assay (WST-1 based) was used to assess cell proliferation (Roche, Cat. No. 05 015 944 001). WST-1 assay used to measure cell proliferation based on the cleavage of tetrazolium salt (WST-1) to formazan by cellular mitochondrial dehydrogenases. Through a colorimetric measurement, the proliferation fold change can be calculated and utilized to determine which cell lines grew significantly more than the negative control, FC550A (grown in serum free media). Cells were serum starved and plated in adherent plates with serum free media. WST-1 measured after 48 hours in culture.

### 8.2.4. Assessment of Myogenicity

Using qRT-PCR (real-time quantitative reverse transcription). Messenger RNA (mRNA) is isolated from cells to examine gene expression with probes specifically designed to amplify select target genes to characterize cell lines. Identical quantity of mRNA is reverse transcribed to generate cDNA. Each cDNA is submitted to quantitative PCR (qPCR) to assess the expression of myogenic factors relative to a housekeeping gene. Expression of MyoD, MyoG, and/or MyHC1e indicate myogenic cells. Additionally, high levels of MyHC1e are indicative of cells that can mature to form myotubes.

Using immunohistochemistry. Cells are seeded in a 96-well plate at a low density (5000-10,000 cells/cm2) to allow cells to grow in the presence or absence of different small molecule combinations. After 2 days of media exposure, cells are fixed with 4% paraformaldehyde (PFA) and washed. Cells are permeabilized with 0.05% PBS-T (triton-x), blocked with normal goat serum (Millipore Sigma) and are incubated with antibodies, and subsequently with secondary antibodies.

### 8.3. Example 1: Promoter Screen Identified hEF1a as Potent Promoter for Driving Expression of Growth Factor Ligand

This experiment was designed to evaluate promoters for their ability to drive expression of the growth factor ligands (and growth factor receptors).

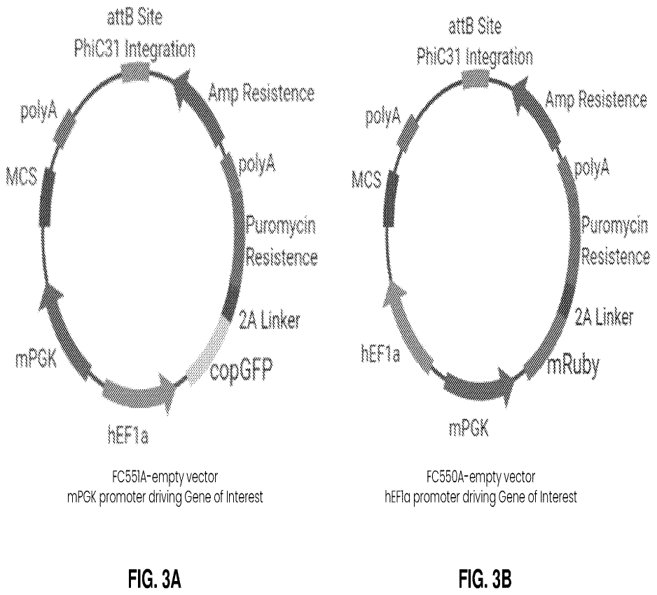

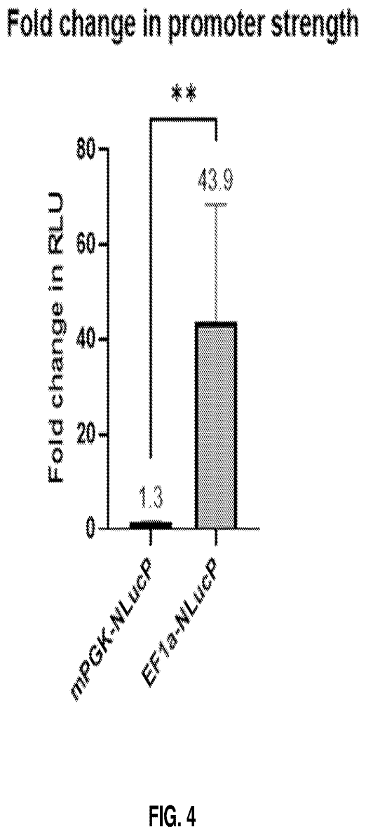

For these experiments, vectors having bi-directional promoters were used (see, e.g., FIGS. 3A and 3B) to assess each promoter's ability to drive expression of FGF2. In particular, the constructs included hEF1alpha and mPGK promoter sequences and a coding sequence of FGF2 or a luciferase reporter gene downstream of the promoter sequences. The vectors shown in FIG. 3A and FIG. 3B were transfected into chicken fibroblast cells. Promoter strength (i.e., the promoter's ability to drive expression of a luciferase reporter gene) was assessed by measuring relative light unit (RLU) fold change relative to mPGK-NLucP control (see FIG. 4). Promoter strength for driving expression of FGF2 was measured by determining the FGF2 concentration in the supernatant after 72 hours in culture.

Surprisingly, as shown in FIG. 4, the hEF1alpha promoter produced statistically significantly greater levels of luciferase reporter gene expression. In particular, the hEF1alpha promoter produced 43.9× higher expression of the luciferase reporter gene than the mPGK promoter. Statistically significant differences between the means were determined by a t-test with P-values indicated with asterisks. N=5. ** p<0.01.

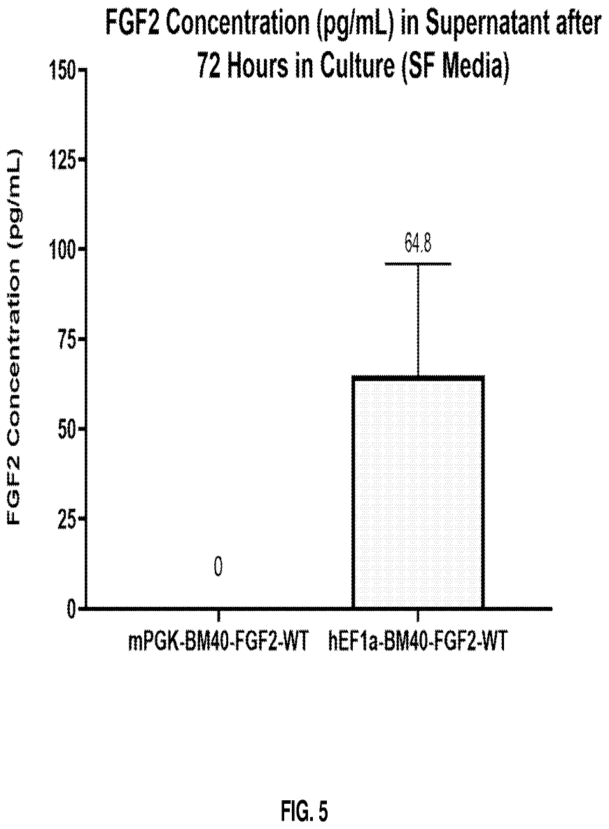

As shown in FIG. 5, the hEF1alpha promoter resulted in a mean of 64.8 pg/mL of FGF2 in the supernatant following 72 hours in culture compared to 0 pg/mL of FGF2 in the supernatant when expression of FGF2 was linked to the mPGK promoter.

This data showed that the hEF1alpha promoter resulted in greater than 60-fold increase in expression compared to the mPGK promoter. For at least these reasons, the hEF1alpha promoter was selected for use in the following experiments.

### 8.4. Example 2: Cells Engineered to Express FGF2 Secrete FGF2 into Culture Media

This experiment was designed to assess performance of different FGF2 variants by measuring FGF2 concentration in the culture media 72 hours post transduction. For these experiments, chicken 1312 cells were transfected, selected using a cell sorter, and expanded prior to assessing FGF2 concentration in the cell culture media.

In particular, chicken 1312 cells seeded in a 6-well plate were transfected using Lipofectamine 3000 using a 1:1 ratio of diluted Lipofectamine 3000 mixture (e.g., 125 μL Opti-Mem and 3.75 μL Lipofectamine) to diluted DNA plasmid mixture (e.g., 125 μL Opti-Mem+5 μL of P3000 reagent+2.5 μg of DNA plasmid). After 48-72 hours, cells were assessed for expression levels using microscopy and flow cytometry (data not shown).

A cell sorter was used to sort the mRuby⁺ cells, which served as a proxy for FGF2 expressing cells. Gates for sorting mRuby⁺ cells were set up using Naïve transfection (no transfection control) (data not shown).

Following sorting, cells were grown in a tissue culture treated 12 well plates. When cells reach 80-90% confluence, the cells were trypsinized and passaged. Cells were maintained in maintenance media (DMEM/F12 and 10% FBS and 1-2% chicken serum).

After the cells recovered from cell sorting, the cells were re-adapted to suspension culture and grown in 50 mL TPP tubes or shake flasks. Cells were seeded at a density of 0.25 1E6 cells/mL and passaged every 3 days. Serum free and ACF media formulation (100 ng/mL FGF2 and 10 ng/mL IGF1) was added to the cultures for continuous growth.

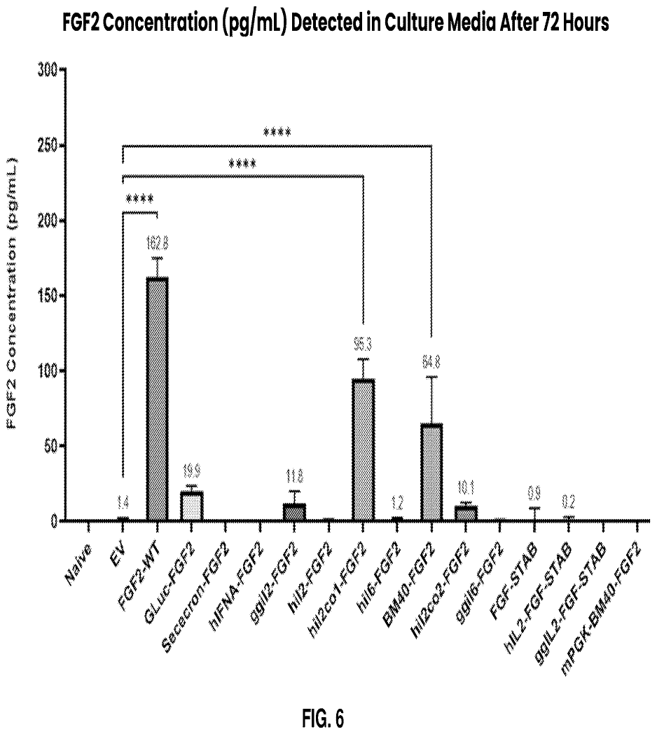

To assess FGF2 concentration in the culture media, an ELISA (enzyme-linked immunoassay) was used. In particular, chicken 1312 cells transduced and sorted according to the above methods were plated in 48-well plates in replicate (N=2) at 60,000 cells/well in 400 uL Serum Free media supplemented with 5 ug/mL Fibronectin. Cells were left to grow at 39° C. for about 72 hours. Supernatant was removed, spun at 300 rcf for 5 minutes and moved to a clean Eppendorf tube. 100 uL of each supernatant was assessed for each well—biological replicates were expanded to technical duplicates resulting in a final N=4 per condition. The results of the FGF2 ELISA are shown in FIG. 6.

Applicant also assessed whether secretion signal peptides (SSPs) could be used to enhance secretion of FGF2. Wild type FGF2 is secreted by a non-conventional secretion mechanism, which does not rely on SSPs. Therefore, Applicant hypothesized that using a SSP fused to the N-terminus of FGF-2 would enhance FGF2 secretion by using conventional secretion pathways (e.g., through the endoplasmic reticulum and golgi apparatus).

As shown in FIG. 6, cells engineered to express FGF2 wild type (FGF2-wt) or engineered to express FGF2 with an N-terminal secretion signal peptide (cohlL2col-FGF2 and BM40-FGF2) secreted statistically significantly levels of FGF2 into the media after 72 hours. Statistically significant differences between the means were determined by an ANOVA one-way test with Dunnett's exclusion (post hoc) with P-values indicated with asterisks. * p<0.05; ** p<0.001; *** p<0.0005; **** p<0.0001; and ns=not significant.

To assess proliferation of the chicken 1312 cells following transduction and sorting (according to the above methods), a WST-1 assay was used. The WST-1 assay used to measure cell proliferation based on the cleavage of tetrazolium salt (WST-1) to formazan by cellular mitochondrial dehydrogenases. Through a colorimetric measurement, the proliferation fold change can be calculated and utilized to determine which cell lines grew significantly more than the negative control, FC550A (grown in serum free media). Cells were serum starved and plated in adherent plates with serum free media. WST-1 measured after 48 hours in culture.

In particular, transfected and sorted chicken 1312 cells were plated in triplicate in 96-well plates in seeding media comprising Serum Free Media (no FGF2). As controls, transfected and sorted chicken 1312 cells were also plated in serum containing media and serum free media having 100 ng/mL FGF2 (i.e., supplemental FGF2). Cells were grown for 48 hours in a stationary incubator at 39° C. N=3 additional wells with a media only were set up as negative controls. Following the 48-hour incubation, 10 uL/well of WST-1 substrate (Roche scientific) was added to each well. Plates were incubated in a stationary incubator for 3 hours. Following incubation, plates were placed on a shaker to ensure mixing for 1 minute. Plates were read on a Cytation Image to determine absorbance (colorimetric output) at 450 nm (630 nm for wavelength correction).

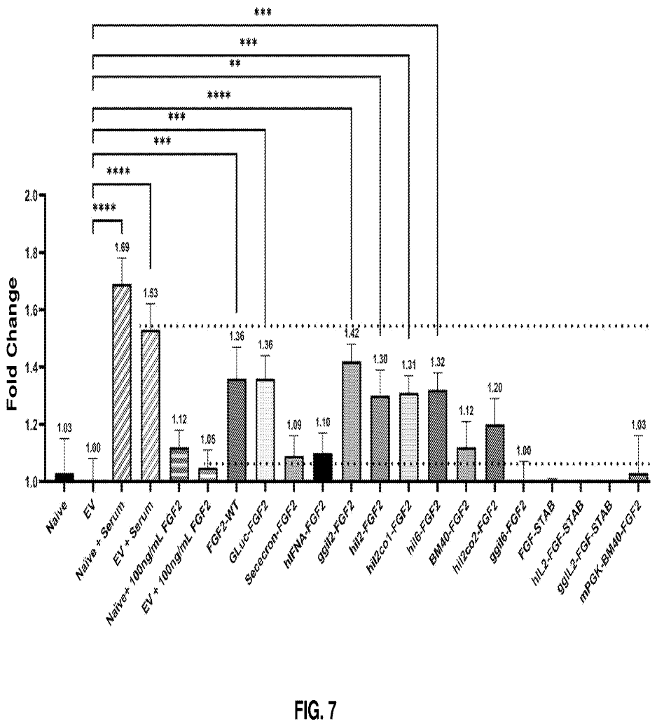

As shown in FIG. 7, ggIL2-FGF2, containing a chicken IL2 secretion signal, had the highest fold change in proliferation compared to FC550A empty vector control (1.42). Statistically significant increases in proliferation were observed for FGF2-WT, Gluc-FGF2, ggIL2-FGF2, hIL2-FGF2, hIL2col-FGF2, and hIL6-FGF2. Controls grown in serum containing media resulted in the highest fold change (positive control). Statistically significant differences between the means were determined by an ANOVA one-way test with Dunnett's exclusion (post hoc) with P-values indicated with asterisks. * p<0.05; ** p<0.001; *** p<0.0005; **** p<0.0001; and ns=not significant.

Taken together, this data showed that cell lines engineered to express FGF2 result in robust production of FGF2, as measured by the concentration of FGF2 in the culture media (FIG. 6). Additionally, the data showed that cell lines engineered to express FGF2 can be grown in serum-free media without the need for supplementing the media with exogenous sources of FGF2 (FIG. 7).

### 8.5. Example 3: Cells Engineered to Express FGF2 Exhibit Significantly Improved Cell Density

This experiment was designed to assess the impact of each FGF2 variant on cell density in adherent, serum-free cultures.

In particular, chicken cells transduced and sorted according to the methods described in Example 2 were plated in 48-well plates in replicates (N=2 wells) at 75,000 cells/well in 400 uL serum-free media supplemented with 5 ug/mL fibronectin. Additional controls included: cell lines not transfected and cell lines transfected with an FC550A empty vector; where the control cell lines were grown in serum-free media supplemented with 100 ng/mL FGF2.

Cells were serum-starved and left to grow at 39° C. for about 72 hours and Biospa imaging was performed every 12 hours to count cells. Cells were then harvested at the end of the experiment and final cell counts were analyzed using a ViCell BLU cell counter.

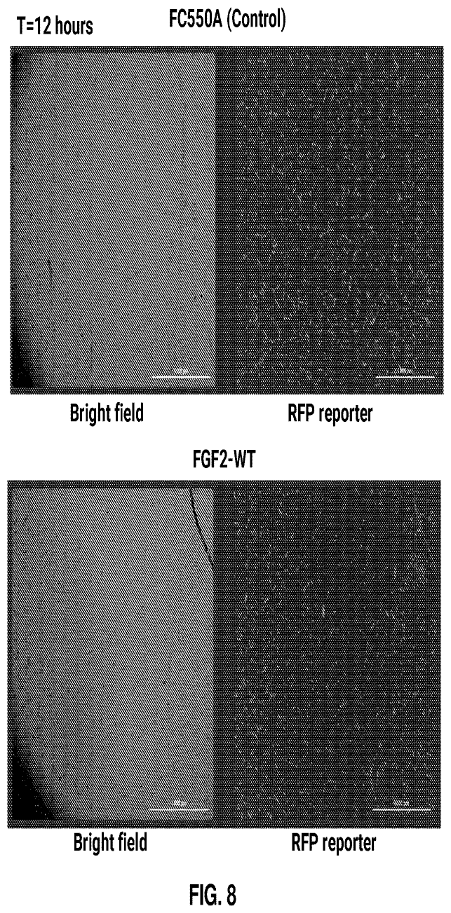

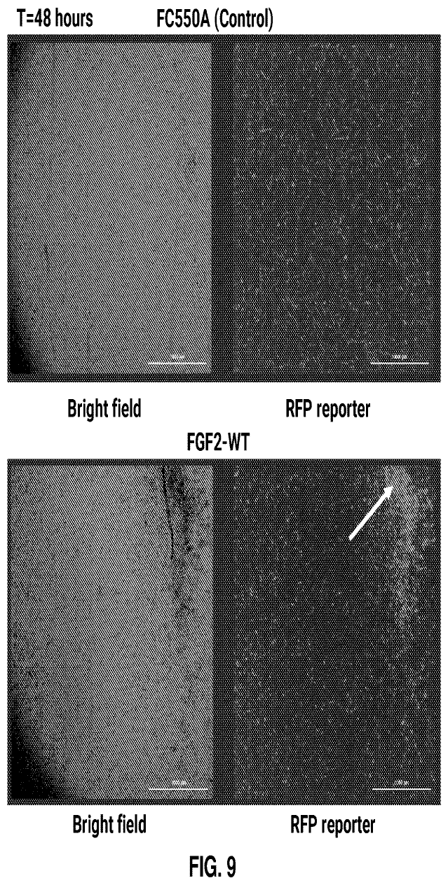

FIG. 8 shows representative images taken at 12 hours after initial seeding of control (FC550A) and FGF2-WT cells plated in serum free media with no exogenous FGF2. FIG. 9 shows representative images taken at 48 hours after initial seeding of control (FC550A) and FGF2-WT cells plated in serum free media with no exogenous FGF2.

As shown in FIG. 9, after 48 hours, multiple cell lines engineered to express FGF2 developed a secondary cell population that was not attached to the plate (identified by the Arrow in FIG. 9) while the FC550A empty vector control consisted only of cells that were adherent.

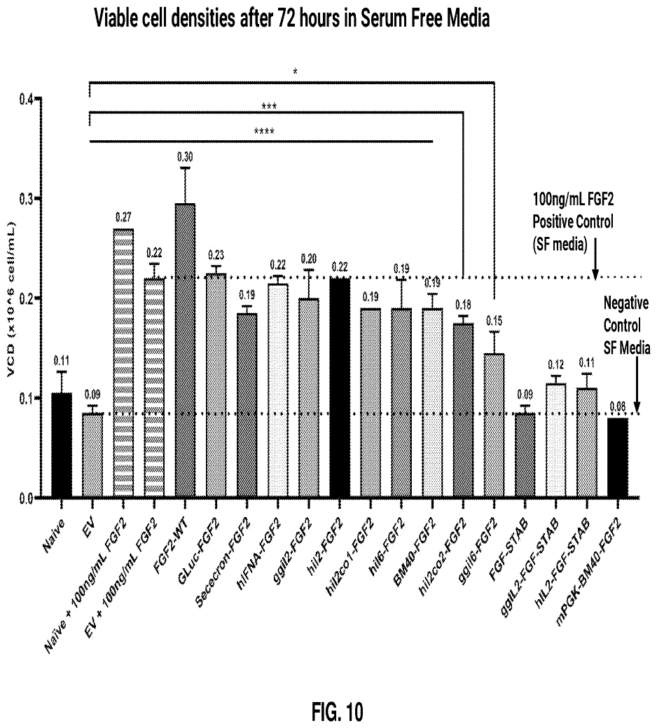

At the end of the 72-hour period, the cells were harvested and viable cell densities were calculated for each condition. As shown in FIG. 10, FGF2-WT transfected cells grew significantly better than the negative controls and similar to positive controls grown in serum free media with 100 ng/mL FGF2. Many FGF-expressing lines were able to grow significantly better than the FC550A empty vector control (Blue bar). Interestingly, all FGF-STAB expressing cell lines were not significant. Statistically significant differences between the means were determined by an ANOVA one-way test with Dunnett's exclusion (post hoc) with P-values indicated with asterisks. * p<0.05; ** p<0.001; *** p<0.0005; **** p<0.0001; and ns=not significant.

This data showed that cell lines engineered to express FGF2 result in cell densities comparable to positive controls, indicating that these cell lines are suitable sources for generating cell-based meat.

### 8.5.1. Further Assessment of Impact on Cell Density

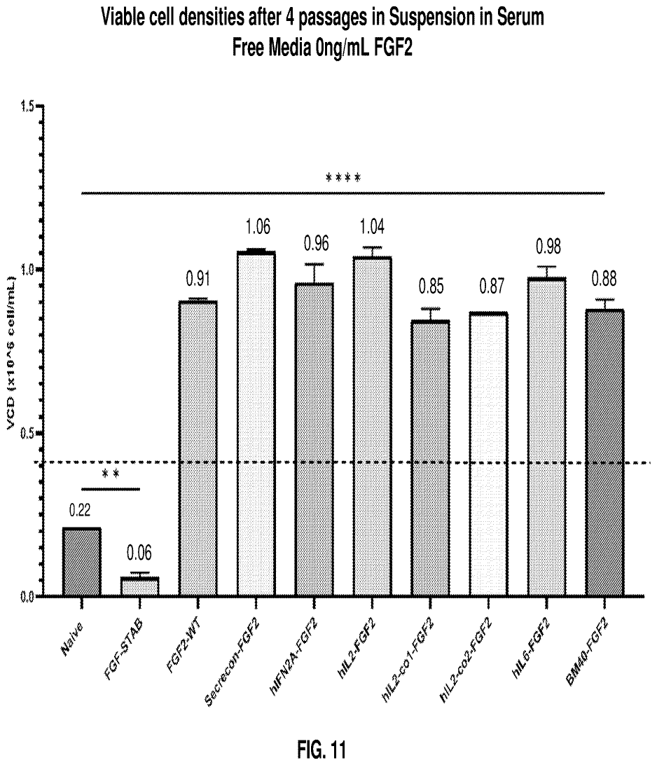

In another experiment, to test impact on cell density, naive cells (none transfected controls) and cells engineered to express FGF2-WT or FGF2-WT with a secretion tag (e.g., a secretion tag selected from Secrecon, human IGN2A, human IL2, human IL2 variation 1, human IL2 variation 2, human IL6, BM40) were adapted into suspension format and seeded into a 4 passage proliferation study. Cells were grown in serum free, ACF media for 4 passages on a 2 day passaging cadence (seeding density ˜400K/mL) as biological replicates. (N=2). Serum free, ACF media contained 10 ng/mL IGF1 and 0 ng/mL FGF2. VCDs measurements were taken on Day 0 and Day 2 for each passage to determine doubling times and maximum VCDs. Naïve and FGF-STAB variants were used as controls. As shown in FIG. 11, after 4 passages, each of the FGF2 variants tested showed statistically significant increases in VCD as compared to the controls. This data established that engineered cells (FGF2 and FGF2 with a secretion tag) were able to grow in suspension without media-supplemented with FGF2. Notably, cells engineered to express thermostable FGF-STAB sequences and naive cells did not proliferate. FIG. 11 shows Viable Cell Density (VCD) of day 2, passage 4. One way ANOVA was performed with significance indicated as P<0.0001(****) and P<0.001 (**) compared to naive control.

### 8.6. Example 4: Cells Engineered to Express FGF2 Variants Enabled Cell Proliferation without Media-Supplemented FGF2

Chicken cells were engineered to express chicken FGF2, bovine FGF2, salmon FGF2, heat stable variants of FGF2 (e.g., FGF2-Q68I-N114G and FGF2-Q68I-C99S-N114G), FGF2 having a secretion tag (e.g., ggIL2-FGF2 and hIL2-col-ggFGF2(xMet)), FGF2 variants that preserve amino acids for non-canonical secretion (FGF2-C77-Y81), and FGF2-STAB variants (STAB-C77Y81, c96-STAB-Gen2-ggFGF2, STAB-Gen3-ggFGF2, and c96-STAB-Gen3-ggFGF2). Controls include cells transduced with an empty vector (EV) and untransfected controls (Naïve).

In a first set of experiments, cells engineered to express FGF2 and FGF2 variants described above were assessed for their ability to proliferate and enable biomass production in media not supplemented with FGF2.

For assessment of impact on proliferation, naïve and engineered cells were grown in suspension in ACF media (10 ug/L IGF1) for 4 passages on a 2 day passaging cadence (initial seeding density 0.5M cells/mL) as biological replicates (N=2). Positive controls included naive and empty vector (EV) engineer cells grown with media supplemented FGF2. Statistics: One way ANOVA was performed to compare test groups against empty vector (EV) grown in FGF2-containing media (100 μg/L): not significant (n.s.), p<0.05 (*), p<0.001(**), p<0.0001(***), and p<0.0001 (****).

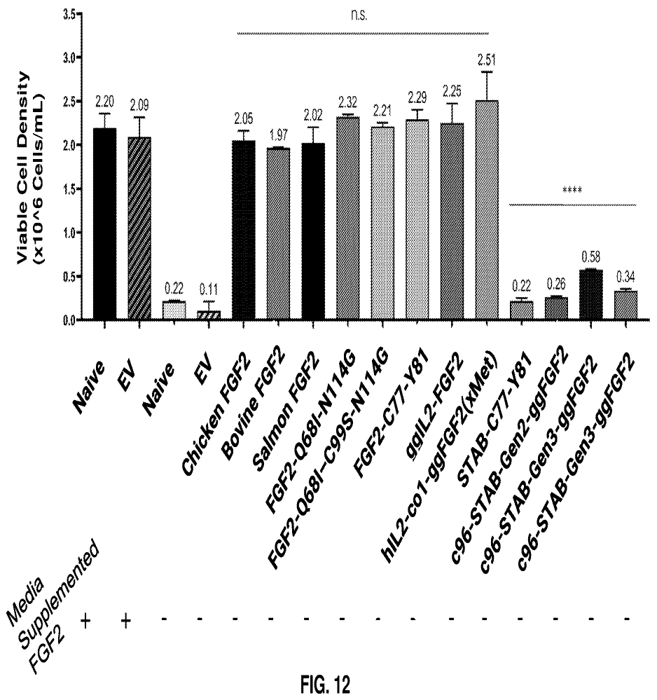

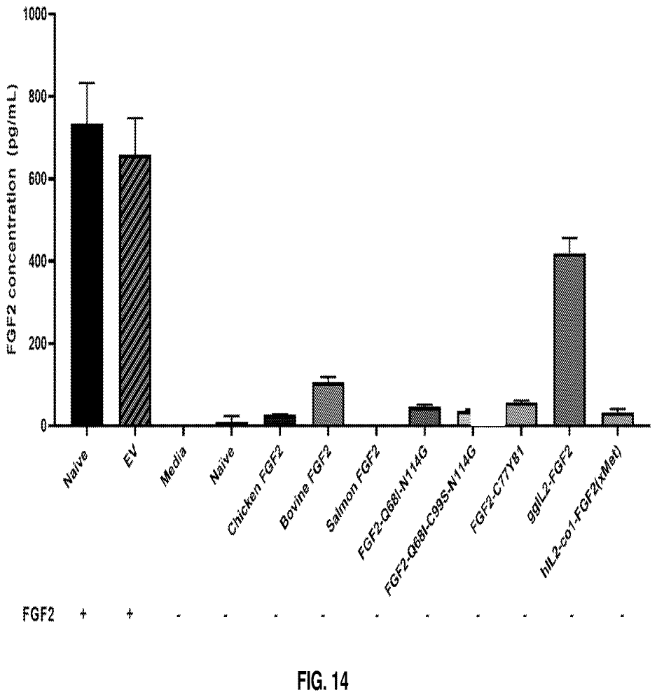

FIG. 12 shows viable cell density data shown for cultures on day 2 of passage 4. This data showed that thermostable FGF2 sequence variations based on computationally designed STAB sequence were unable to promote cell growth in FGF2-free media (FIG. 14). In contrast, each of the other variants of FGF2 resulted in VCDs similar to the controls.

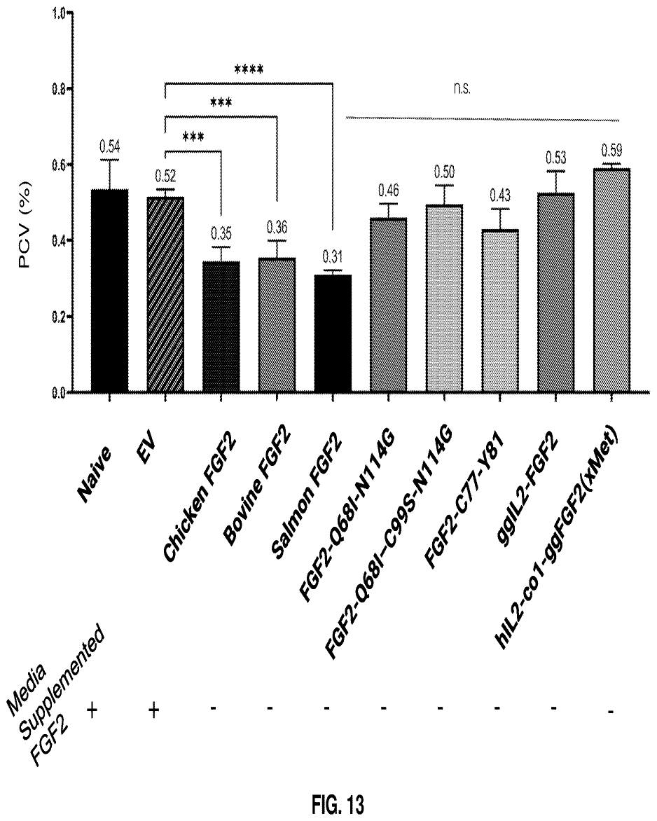

For assessment of impact on biomass production, naïve and engineered cells were grown in suspension for 5 passages on a 2 day passaging cadence in media containing IGF1 (10 μg/L). Seeding density at each passage was 0.5M cells/mL; N=2. Naive and empty vector negative controls grown without media-supplemented FGF2 and did not survive to passage 5 (data not shown). On Day 2 of passage 5, packed cell volume (PCV) was taken by spinning 500 μL of culture at 1000×g for 5 minutes and collecting a PCV reading. The PCV reading can be converted to percent PCV by the following formula: % PCV=(PCV reading/sample volume)*100. Statistics: One way ANOVA was performed to compare test groups against empty vector (EV) grown in FGF2-containing media (100 μg/L): Not significant (n.s.), p<0.05 (*), p<0.001(**), p<0.0001(***)p<0.0001 (****). Biomass production data (i.e., percent packed cell volumes) is shown in FIG. 13.

FIG. 13 shows percent packed cell volume for cultures on day 2 of passage 5. FIG. 13 shows that media supplemented with FGF2 was not required for the eight engineered cell lines to achieve packed cell volumes similar to the controls. The lower PCV's observed for chicken, bovine, and salmon FGF2 cell lines may be attributed to variations in the polyclonal population.

The engineered cells lines were assessed for their ability to secrete FGF2. An ELISA was performed on spent media (supernatants) from cell lines grown in suspension media (10 μg/L IGF1) with/without media supplemented FGF2 (100 μg/L); biological replicates N=2. Positive controls included spent media from naive and empty vector (EV) cells grown in FGF2 supplemented media. Negative controls included base media (Media) and spent media from naive cells grown in media not supplemented with FGF2. On Day 2 of Passage 5, cells were spun at 300×g for 5 minutes and the supernatant was removed for use in FGF2-ELISA kit to evaluate the concentration of FGF2 in the supernatant. For each condition, biological replicates (N=2) and technical replicates (N=4) were analyzed. FIG. 14 shows FGF2 concentration (pg/mL) in supernatants collected from the controls and the engineered cells.

Overall, this data showed that cells engineered to express FGF2 or variants of FGF2 could be grown in the absence of FGF2, exhibit robust growth as compared to controls, thereby showing that the engineered cells can be grown in culture media not supplemented with FGF2. Therefore, these cells lines are ideal for use in cell based meat products suitable for consumption because they can endure the requisite culture conditions (i.e., extensive passaging) needed to make these products all while reducing the costs associated with using the vast amounts of exogenous growth factors traditionally needed to make these products.

### 8.7. Example 5: Cell Lines Engineered to Express FGF Receptor Variants Proliferate without Media Supplemented FGF2

Chicken cells were engineered to express FGF receptors, including FGF1 receptors, FGF2 receptors, FGF3 receptors, and FGF4 receptors. FGF1 receptors included: FGFR1C, FGFR1C-N546K, FGFR1C-V561M, and myrist-FGFR1C-K656E. FGF2 receptors included: FGFR2, FGFR2-N550K, FGFR2-K660E, and FGFR2-3xMuts. FGF3 receptors included: FGFR3, FGFR3-N540K, FGFR3-K560E, FGFR3-N540K-K560E, and myrist-FGF3R-K560E. FGF4 receptors included: FGF4R, FGF4R-Y367C, and FGF4R-Y367C-K654E.

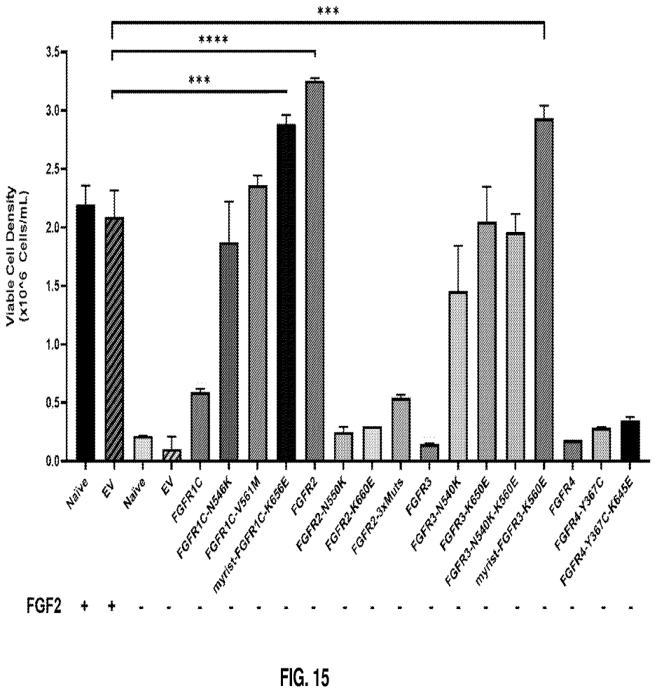

For assessment of impact the FGF receptors on proliferation, cells were grown over 4 passages in suspension culture with ACF media containing IGF1 (10 ug/L) and with/without FGF2 (100 ug/L) on a 2 day passaging cadence (seeding density 0.5M cells/mL) as biological replicates (N=2). Viable Cell Density (VCD) was taken on day 2 of passage 4 of culture (see FIG. 15). One way ANOVA was performed to compare test groups against empty Vector (EV) grown in FGF2-containing media (100 ug/L), Not significant (n.s.), p<0.05 (*), p<0.001(**), p<0.0001(***), and p<0.0001 (****).

As shown in FIG. 15, three cell lines including myrist-FGFR1C-K656E, FGFR2 (wild type), myrist-FGFR3-K560E achieved higher viable cell densities in FGF2-free media than positive controls grown with FGF2. Multiple conditions archived growth comparable to Positive Controls including FGFR1C-N546K, FGFR1C-V561M, FGFR3-N540K, FGFR2-K650E, and FGFR3-N540K-K560E.

For assessment of impact of the FGF receptors on biomass production, cells were grown in suspension for 5 passages on a 2 day passaging cadence in media containing IGF1 (10 μg/L). Seeding density at each passage was 0.5M cells/mL; N=2. Naive and empty vector negative controls grown without media-supplemented with FGF2 did not survive to passage 5 (data not shown). On Day 2 of passage 5, packed cell volume (PCV) was taken by spinning 500 μL of culture at 1000×g for 5 minutes and collecting a PCV reading. The PCV reading can be converted to percent PCV by the following formula: % PCV=(PCV reading/sample volume)*100. One way ANOVA was performed to compare test groups against empty Vector (EV) grown in FGF2-containing media (100 ug/L), Not significant (n.s.), p<0.05 (*), p<0.001(**), p<0.0001(***), and p<0.0001 (****).

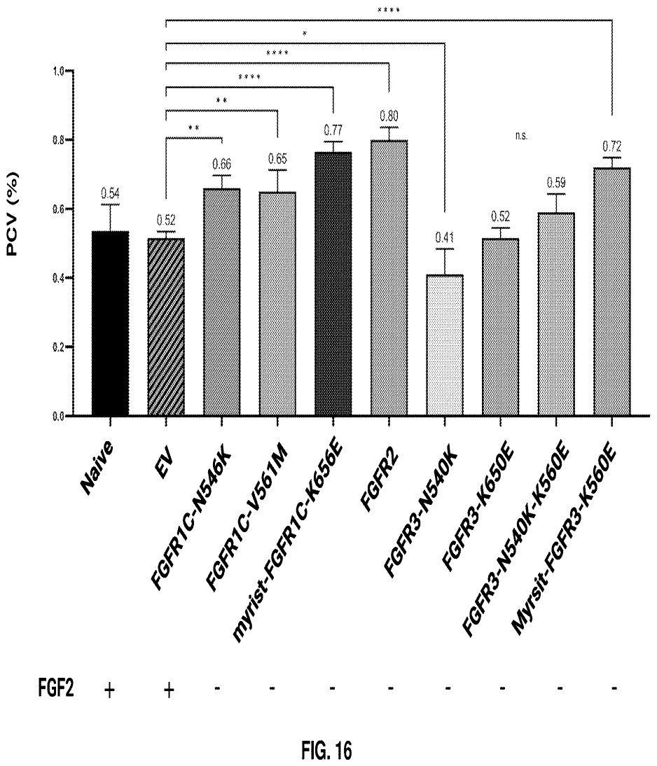

As shown in FIG. 16, shows that media supplemented with FGF2 was not required for the eight engineered cell lines to achieve packed cell volumes similar to the controls.

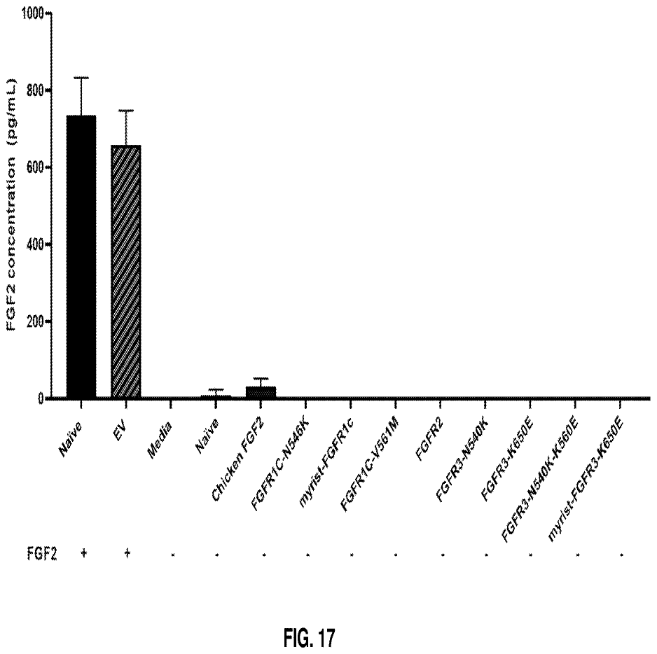

The engineered cells lines were assessed for their ability to secrete FGF2. An ELISA was performed on spent media (supernatants) from cell lines grown in suspension media (10 μg/L IGF1) with/without media supplemented FGF2 (100 μg/L); biological replicates N=2. Positive controls included spent media from naive and empty vector (EV) cells grown in FGF2 supplemented media. Negative controls included base media (Media) and spent media from naive cells grown in media not supplemented with FGF2. On Day 2 of Passage 5, cells were spun at 300×g for 5 minutes and the supernatant was removed for use in FGF2-ELISA kit to evaluate the concentration of FGF2 in the supernatant. For each condition, biological replicates (N=2) and technical replicates (N=4) were analyzed. FIG. 17 shows FGF2 concentration (pg/mL) in supernatants collected from the controls and the engineered cells.

Overall, this data showed that cells engineered to express FGF1 receptor, FGF2 receptors, FGF3 receptors, or FGFR4 receptors (or variants thereof) could be grown in the absence of FGF2, exhibit robust growth as compared to controls, thereby showing that the engineered cells can be grown in culture media not supplemented with FGF2. Therefore, these cells lines are ideal for use in cell-based meat products and cell-based food products suitable for consumption because they can endure the requisite culture conditions (i.e., extensive passaging) needed to make these products all while reducing the costs associated with using the vast amounts of exogenous growth factors traditionally needed to make these products.

### 8.8. Summary of Examples 2-5

Overall, this data suggests that cell lines engineered to express FGF2-WT, or cells engineered to express FGF2-WT with various secretion signals, and grown in the absence of FGF2, exhibit robust growth as compared to controls, thereby showing that the engineered cells can be grown in culture media not supplemented with FGF2. Therefore, these cells lines are ideal for use in cell-based meat products and cell-based food products suitable for consumption because they can endure the requisite culture conditions (i.e., extensive passaging) needed to make these products all while reducing the costs associated with using the vast amounts of exogenous growth factors traditionally needed to make these products.

### 8.9. Example 6: Cells Engineered to Express IGF1 and IGF1 Receptors (or Variants Thereof) Significantly Improve Cell Density and Packed Cell Volumes

[498] This example was designed to assess how expression of exogenous IGF1 impacted cell density and packed cell volume. This experiment was also designed to assess how cells characterized as IGF1^{low} or IGF1^{high} contributed to cell density and packed cell volume.

### 8.9.1. Assessment of IGF1 and IGF1 Receptor Variants

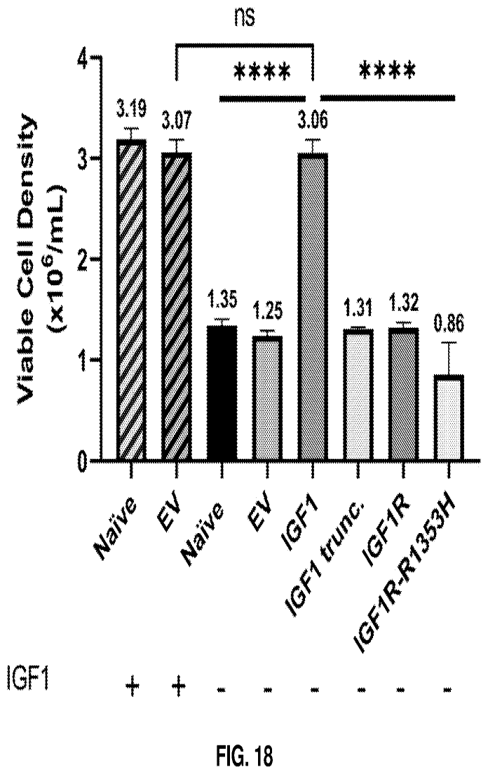

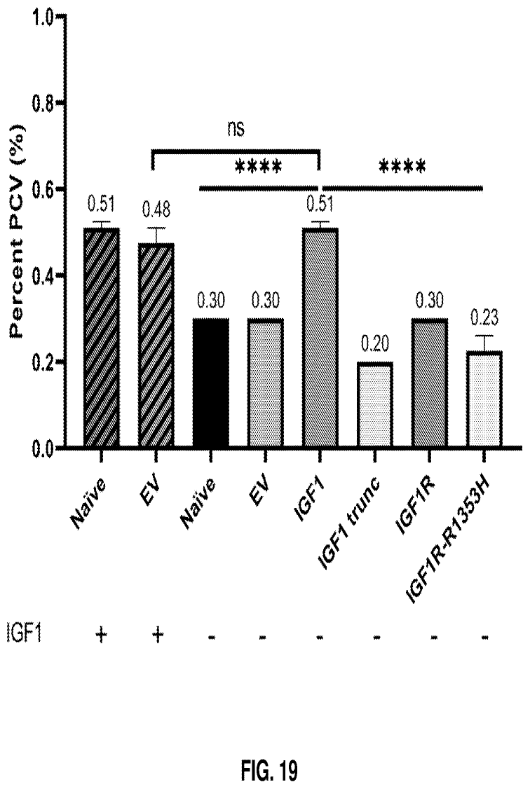

In a first set of experiments, Chicken cells were engineered to express IGF1 WT (SEQ ID NO: 16), IGF1-trunc., IGF1R, or IGF1R-R1353H. These cells were grown in suspension with ACF media (100 ug/L FGF2) without media supplemented IGF1. Cells were seeded at 0.5E6 cells/mL (N=2) and passaged every 2 days over 4 passages. Viable cell density (FIG. 18) and packed cell volume (PCV (FIG. 19) were assessed to help determine how the engineered cells performed compared to controls.

Viable Cell Density (VCD) of day 2, passage 4 is provided in FIG. 18. One way ANOVA performed compared to IGF1 expressing cell line grown in IGF1-free media, Not significant (n.s.), p<0.05 (*), p<0.001(**), p<0.0001(***), and p<0.0001 (****). As shown in FIG. 18, expression of IGF1 receptor variations or truncated IGF1 was not able to boost cell growth in IGF1-free media. In contrast, cells engineered to express wild type IGF-1 produced VCD similar to the controls (FIG. 18).

Packed cell volume (PCV) of day 2, passage 3 is provided in FIG. 19. PCV determined by spinning 500 μL of culture at 1000×g for 5 minutes and collecting a PCV reading. The PCV reading was converted to percent PCV by the following formula: % PCV=(PCV reading/sample volume)*100. One way ANOVA performed compared to IGF1 expressing cell line grown in IGF1-free media, Not significant (n.s.), p<0.05 (*), p<0.001(**), p<0.0001(***), and p<0.0001 (****). As shown in FIG. 19, expression of IGF1 receptor variations or truncated IGF1 was not able to boost cell growth in IGF1-free media. In contrast, cells engineered to express wild type IGF-1 produced PCV similar to the controls (FIG. 19).

This data showed that chicken cells engineered to express IGF1 yield produced the most favorable cell density and packed cell volume among the conditions tested. Chicken cells engineered to express IGF1 were selected for additional analysis.

8.9.2. Assessment of IGF1^{low} and IGF1^{high} Cells

In another set of experiments, chicken cells were engineered to express either an FC550A empty vector (“EV”) control or a Phi31 vector containing an IGF1 wild type (IGF1-WT) sequence (SEQ ID NO: 16). Transduced cells were sorted into “low” expressing cells and “high” expressing cells based on RFP expression. RFP expression served as a proxy for IGF1 expression. “Low-expression” cells and “high-expression” cells were separately plated in suspension format in TPP tubes. Controls included: cell lines not transfected (“Naïve”) and the EV control; where the control cell lines were grown in serum-free media supplemented with and without IGF1.

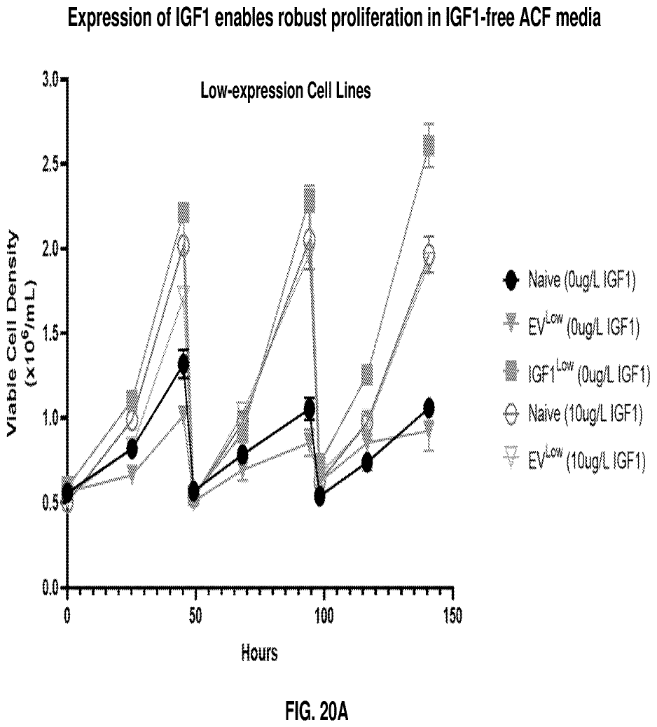

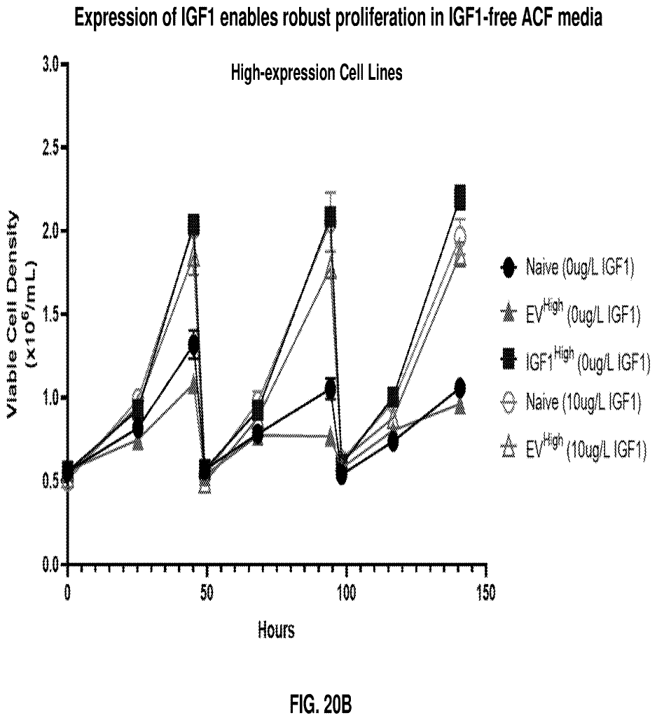

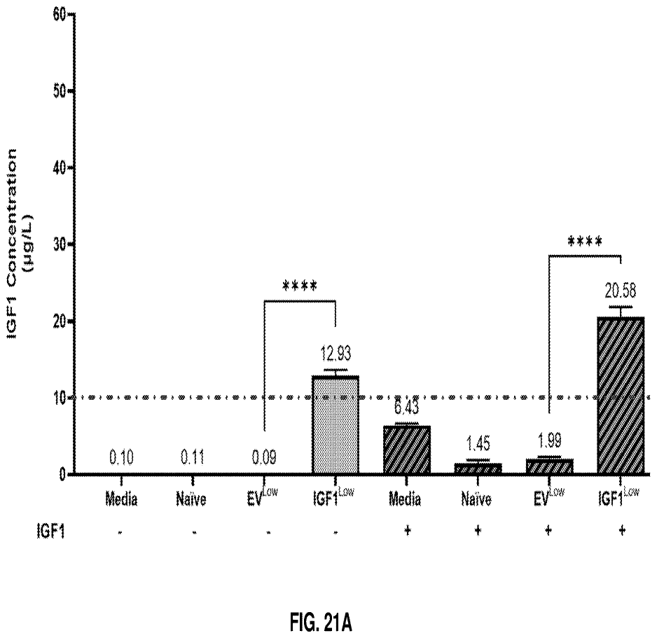

FIGS. 20A-20B shows growth curves over 3 passages for cell lines grown in ACF media supplemented with 0 μg/L or 10 μg/L IGF1. Here, cells were seeded at 0.5E6 cells/mL and passaged on a 2-day cadence. Cell counts were taken daily using ViCell BLU. Each condition was grown as a biological replicate (N=2).

As shown in FIG. 20A, cells engineered to expressed low levels of IGF1 (IGF1^{Low}) grown in 0 μg/L IGF1 had robust growth curves as compared to controls (i.e., Naïve and EV) not grown in the presence of IGF1. In fact, the cells engineered to express IGF1 (IGF1^{Low}) had growth curves similar to the controls (i.e., Naïve and EV) grown in 10 μg/L IGF1 (see FIG. 20A). This data suggests that IGF1^{Low} cells are able to produce enough of IGF1 such that supplementing the culture media with IGF1 is not required.

FIG. 20B show cells engineered to expressed high levels of IGF1 (IGF1^{High}) grown in 0 μg/L IGF1 had robust growth curves as compared to controls (i.e., Naïve and EV) not grown in the presence of IGF1. Similar to the IGF1^{Low} cells, the cells engineered to express high levels of IGF1 (IGF1^{High}) had growth curves similar to the controls (i.e., Naïve and EV) grown in 10 μg/L IGF1 (see FIG. 20B). This data suggests that IGF1^{High} cells are able to produce enough of IGF1 such that supplementing the culture media with IGF1 is not necessary.

The IGF1^{low} and IGF1^{high} cultures were assessed for their ability to secrete IGF1 into the culture medium. For these experiments, an ELISA was performed on spent media (supernatants) from cell lines grown in ACF media (100 ug/L FGF2) with/without media supplemented IGF1 (10 μg/L). Positive controls included spent media from Naïve cells, empty vector (EV) cells grown in IGF1 supplemented media, and EV cells grown in base media (100 ug/L FGF2). Negative controls included base media (Media) and spent media from naive cells grown without IGF1. On Day 2 of Passage 3, cells were spun at 300×g for 5 minutes and the supernatant was removed for use in IGF1-ELISA kit to evaluate the concentration of IGF1 in the supernatant. For each condition, biological replicates (N=2) and technical replicates (N=4) were analyzed. One way ANOVA was performed to compare to IGF1 expressing cell lines to empty vector (EV) controls: not significant (n.s.), p<0.05 (*), p<0.001(**), p<0.000 (***)p<0.0001 (****).

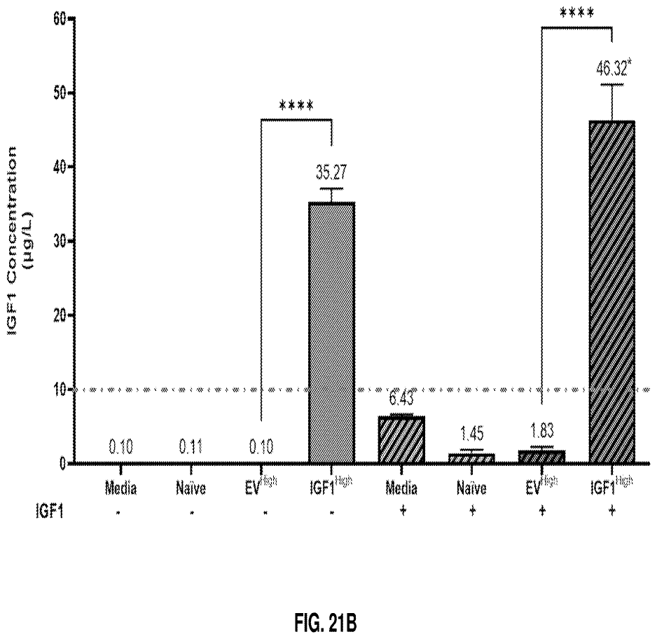

FIG. 21A shows IGF1 ELISA data for IGF1^{low} cells. Supernatants from IGF1^{low} cells had 12.93 μg/L of IGF1 when grown in media not supplemented with IGF1 and 20.58 μg/L of IGF1 when grown in media supplemented with IGF1 (see FIG. 21A). FIG. 21B shows IGF1 ELISA data for IGF1^{high} cells. Supernatants from IGF1^{high} cells had 35.27 μg/L of IGF1 when grown in media not supplemented with IGF1 and 45.32 μg/L of IGF1 when grown in media supplemented with IGF1 (see FIG. 21B). Overall, this data showed robust IGF1 secretion into the culture media by the engineered cells, and the results were as expected with IGF1^{low} secreting lower amounts of IGF1 than IFG1^{high}.

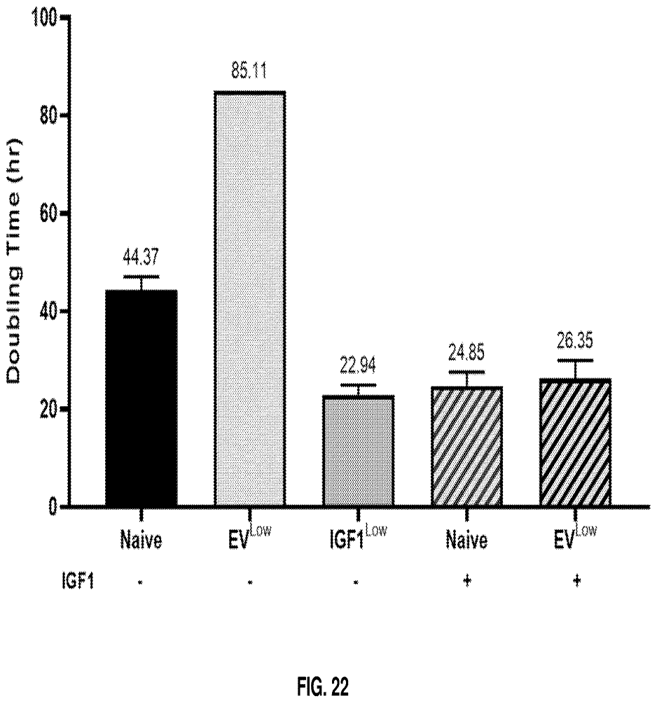

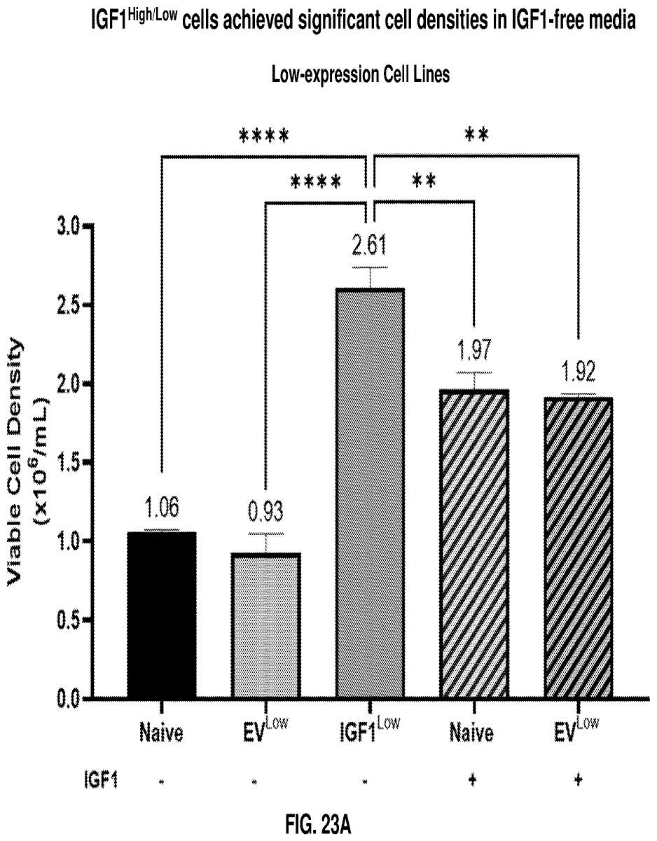

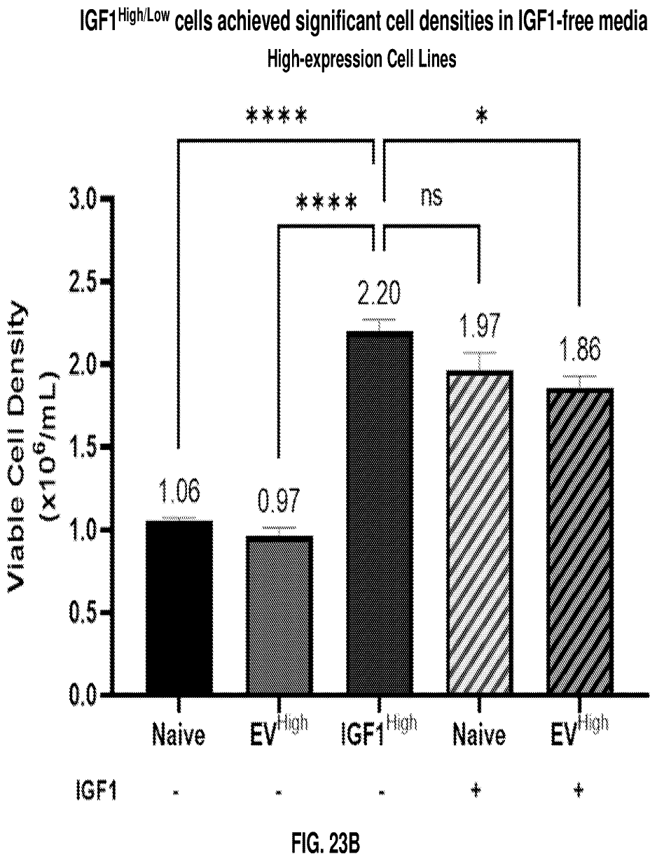

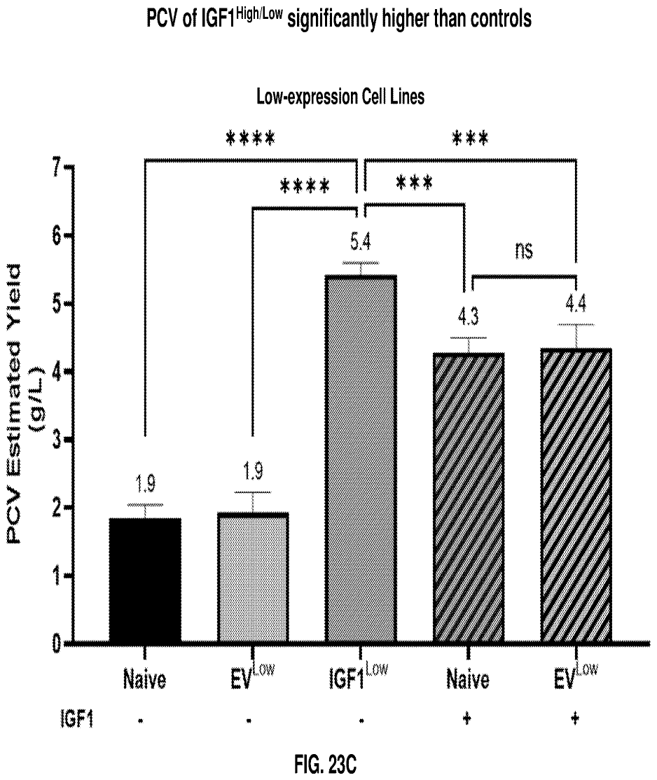

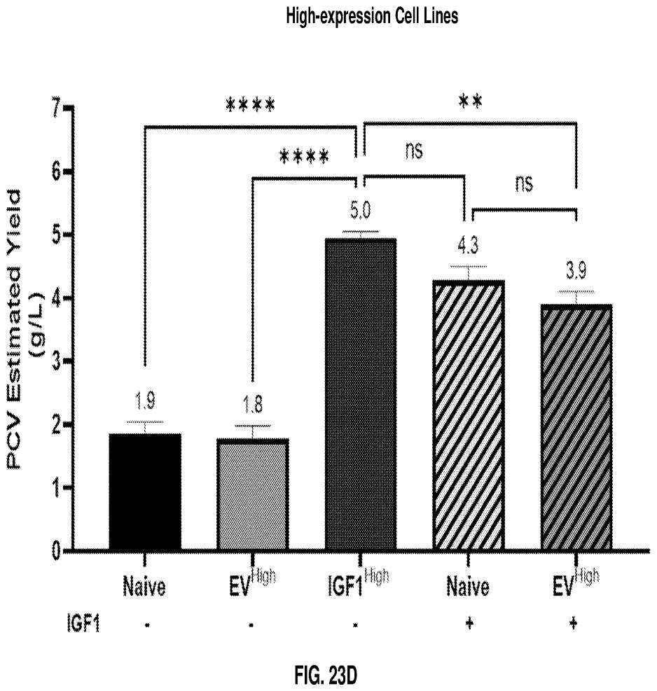

IGF1^{low} cells were assessed for population doubling time (FIG. 22), viable cell density (FIG. 23A) and packed cell volume (FIG. 23C). IGF1^{high} cells were assessed for viable cell density (FIG. 23B) and packed cell volume (FIG. 23D).

FIG. 22 shows doubling time (hours) for chicken cells engineered to express IGF1 (IGF1^{low}) grown in media supplement without (“−”) and with (“+”) IGF1. Cells were grown with or without 10 μg/L IGF1 in ACF media (100 ug/L FGF2) over 3 passages. Cells seeded at 0.5E6 cells/mL and passaged on a 2-day cadence (N=2). Doubling time was calculated by applying exponential growth equation Y=Y0*exp(k*x) to proliferation profiles obtained on the third passage. Where Y0 is the starting population, k is the rate constant, and Doubling Time (hr) is the time need for the population to double, calculated as ln(2)/k. Where Y0 is the starting population, k is the rate constant, and Doubling Time (hr) is the time need for the population to double, calculated as ln(2)/k. One way ANOVA was performed: not significant (n.s.), p<0.05 (*), p<0.001(**), p<0.0001(***), and p<0.0001 (****). As shown in FIG. 22, the addition of media-supplemented IGF1 to IGF1^{Low} culture did not impact cell doubling time.

Further analysis of the suspension cultures at day 2 during the third passage revealed that both IGF1^{low} cells (FIG. 23A) and IGF1^{high} cells (FIG. 23B) had statistically significant differences in viable cell densities (VCD) as compared to the controls. In particular, IGF1^{Low} cells had statistically significant greater VCDs as compared to controls (i.e., Naïve and EV) grown in either 0 μg/L IGF1 or 10 μg/L IGF1 (see FIG. 23A). Similarly, IGF1^{High} cells had statistically significant greater VCDs as compared to both controls (i.e., Naïve and EV) grown in 0 μg/L IGF1 but was only statistically significant as compared to the EV control when controls were grown in 10 μg/L IGF1 (see FIG. 23B). Statistically significant differences between the means were determined by an ANOVA one-way test with Dunnett's exclusion with P-values indicated with asterisks. * p<0.05; ** p<0.001; *** p<0.0005; **** p<0.0001; and ns=not significant.

Applicant then looked at Pack Cell Volumes (PCV), which is a proxy for cell biomass. PCV analysis for the suspension cultures at day 3 during the third passage revealed that both IGF1^{low} cells (FIG. 23C) and IGF1^{high} cells (FIG. 23D) had statistically significant differences in PCV as compared to the controls. To measure PCV, 500 μL of culture volume was collected in a small PCV tube and centrifuged at 1000×g for 5 minutes. Each condition was measured in duplicate (biological replicates with technical replicates N=4).

As shown in FIG. 23C, IGF1^{low} cells had statistically significant greater PCVs as compared to controls (i.e., Naïve and EV) grown in either 0 μg/L IGF1 or 10 μg/L IGF1. IGF1^{high} cells had statistically significant greater PCVs as compared to both controls (i.e., Naïve and EV) grown in 0 μg/L IGF1 but was only statistically significant as compared to the EV control when controls were grown in 10 μg/L IGF1 (see FIG. 23D). Statistically significant differences between the means were determined by an ANOVA one-way test with Dunnett's exclusion with P-values indicated with asterisks. * p<0.05; ** p<0.001; *** p<0.0005; **** p<0.0001; and ns=not significant.

Overall, FIGS. 23C-23D show that cells engineered to express IGF1 (e.g., IGF1^{Low} and IGF1^{High}) actually outperformed (i.e., enable greater PCV) cells grown in the presence of IGF1. As PCV is a proxy for cell biomass, this data suggest that the engineered cells are ideal for use in cell-based meat products and cell-based food products suitable for consumption because they produce the amounts of cell biomass needed for these products and can do so in the absence of exogenous IGF1.

### 8.9.3. Assessment of Impact of Media Supplemented IGF1 on IGF1^{low} Cultures

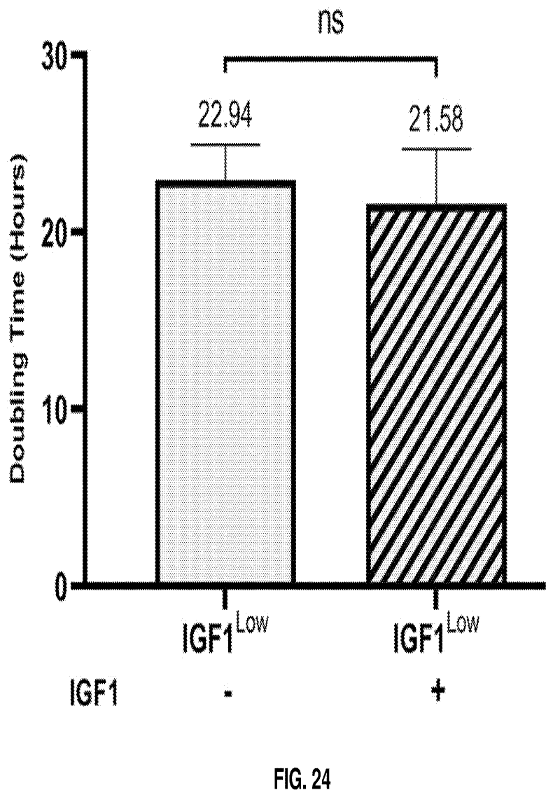

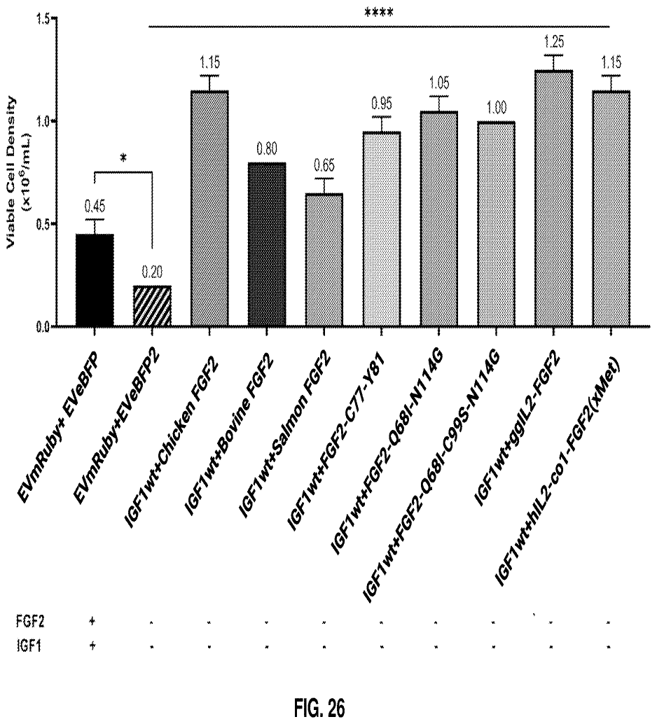

FIG. 26 shows doubling time (hours) for chicken cells engineered to express IGF-1 (IGF1^{low}) grown in media supplement without (“−”) and with (“+”) IGF1. Cells were grown with or without 10 μg/L IGF1 in ACF media (100 ug/L FGF2) over 3 passages. Cells seeded at 0.5E6 cells/mL and passaged on a 2-day cadence (N=2). Doubling time was calculated by applying exponential growth equation Y=Y0*exp(k*x) to proliferation profiles obtained on the third passage. Where Y0 is the starting population, k is the rate constant, and Doubling Time (hr) is the time need for the population to double, calculated as ln(2)/k. Where Y0 is the starting population, k is the rate constant, and Doubling Time (hr) is the time need for the population to double, calculated as ln(2)/k. One way ANOVA was performed: not significant (n.s.), p<0.05 (*), p<0.001(**), p<0.0001(***), and p<0.0001 (****). As shown in FIG. 24, the addition of media-supplemented IGF1 to IGF1^{Low} culture did not impact cell doubling time.

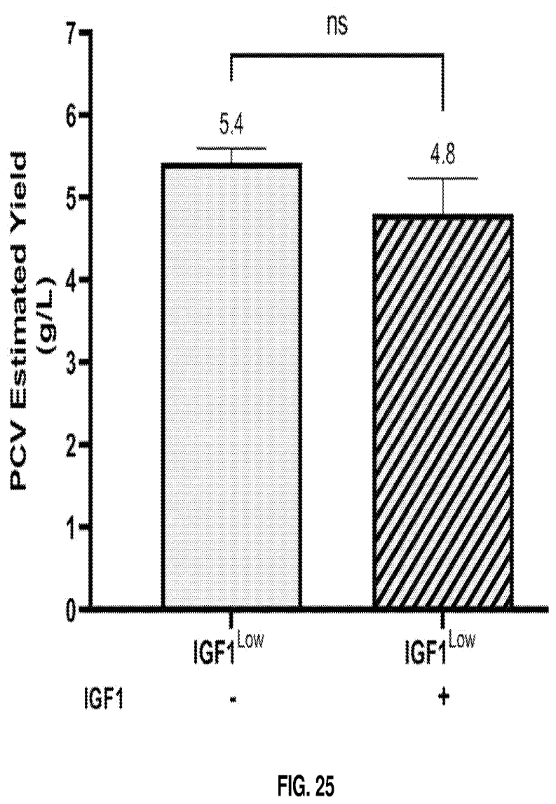

FIG. 25 shows biomass (as PCV (g/L)) for chicken cells engineered to express IGF-1 (IGF1^{low}) grown in media supplement without (“−”) and with (“+”) IGF1. Cells were grown with or without 10 μg/L IGF1 in ACF media (100 ug/L FGF2) over 3 passages. Cells seeded at 0.5E6 cells/mL and passaged on a 2-day cadence (N=2). PCV was measured on Day 2 of Passage 3 using 500 μL of culture volume and centrifuged at 1000×g for 5 minutes. One way ANOVA performed, Not significant (n.s.), p<0.05 (*), p<0.001(**), p<0.0001(***), p<0.0001 (****). One way ANOVA was performed: Not significant (n.s.), p<0.05 (*), p<0.001(**), p<0.0001(***)p<0.0001 (****). As shown in FIG. 25, the addition of media-supplemented IGF1 to IGF1^{Low} culture did not impact PCV.

Taken together, FIGS. 20A-20B, FIG. 21A-21B, FIG. 22, FIGS. 23A-23D, FIG. 24, and FIG. 25 show that IGF1^{Low} and IGF1^{High} cells not only exhibit robust growth independent of exogenous IGF1 (i.e., media supplemented with IGF1) but also outperform (i.e., produce higher VCD) cells grown in the presence of IGF1. This data suggests these cells lines are ideal for use in cell-based meat products and cell-based food products suitable for consumption because they can endure the requisite culture conditions (i.e., extensive passage) needed to make these products—all while reducing the costs associated with using the vast amounts of exogenous growth factors traditionally needed to make these products.

### 8.10. Example 7: Cells Engineered for Complete Growth Factor-Independence Using IGF1 and FGF2

In this example chicken cells were engineered to assess whether expression of IGF1 WT and an FGF2 variant enables growth-factor independence (no requirement for growth factors in culture media).

Chicken cells engineered to express IGF1 WT (IGF1 amino acid sequence of were engineered with an FC550A empty vector (“EV”) control or a Phi31 vector containing one or more FGF2 variants, including chicken (Chicken FGF2), bovine (Bovine FGF2), salmon (Salmon FGF2), heat stable variants (FGF2-Q68I-N114G, FGF2-Q68I-C99S-N114G), secretion tagged variants (ggIL2-FGF2, hIL2-col-ggFGF2(xMet)) or variants that preserve amino acids for non-canonical secretion (FGF2-C77-Y81)). Controls consisted of dual empty vector (EVmRuby and EVeBFP2) that were used to construct the IGF1 and FGF2 variants, respectively, and were grown with or without growth factors IGF1 (10 μg/L) and FGF2 (100 μg/L) (see FIG. 26). Cells were grown in suspension over 4 passages using a 2 day passaging cadence at initial seeding density of 0.5E6 cells/mL (N=2). One way ANOVA performed compared to EV control in growth factor-free media, Not significant (n.s.), p<0.05 (*), p<0.001(**), p<0.0001(***), and p<0.0001 (****).

As shown in FIG. 26, each cell line expressing IGF1 WT and a FGF2 variant resulted in increased viable cell density as compared to the controls. Overall, this data showed that cell lines engineered to express IGF1 WT and a FGF2 variant significantly increased the viable cell density as compared to controls.

### 8.11. Example 8: Cells Engineered for Complete Growth Factor-Independence Using IGF1 and a FGF2 Receptor

In this example chicken cells were engineered to assess whether expression of IGF1 WT and an FGF2 receptor variant enables growth-factor independence (no requirement for growth factors in culture media).

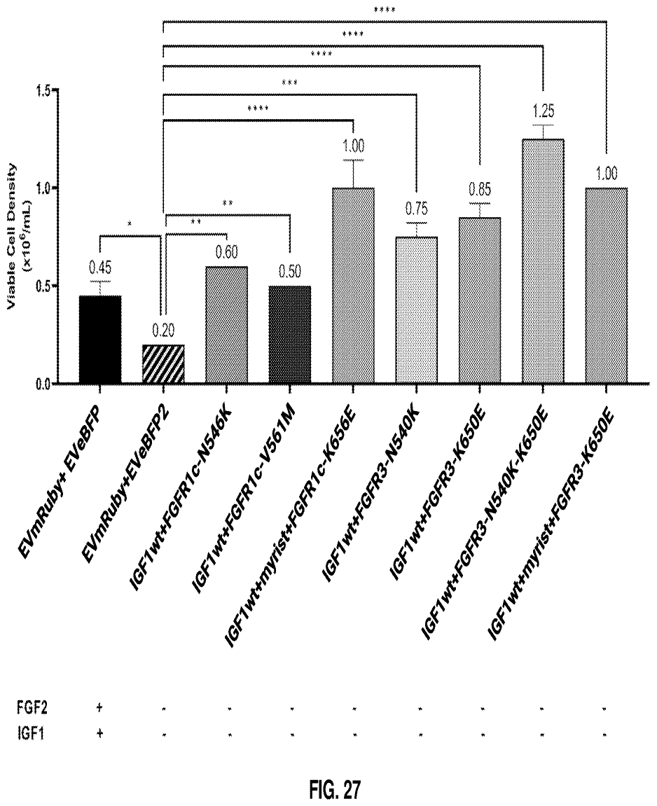

Chicken cells engineered to express IGF1 WT (IGF1 amino acid sequence of were transduced with an FC550A empty vector (“EV”) control or a Phi31 vector containing one or more of: FGFR1c-N546K, FGFR1c-V561M, myrist-FGFR1c-K656E, FGFR3-N540K, FGFR3-K650E, FGFR3-N540K-K650E, or myrist-FGFR3-K650E. Controls consisted of dual empty vector (EVmRuby and EVeBFP2) that were used to construct the IGF1 and FGF2 variants, respectively, and were grown with or without growth factors IGF1 (10 μg/L) and FGF2 (100 μg/L) (see FIG. 27). Cells were grown in suspension over 3 passages using a 2 day passaging cadence at initial seeding density of 0.5E6 cells/mL (N=2). One way ANOVA was performed compared to EV control in growth factor-free media: not significant (n.s.), p<0.05 (*), p<0.001(**), p<0.0001(***), and p<0.0001 (****).

As shown in FIG. 27, each cell line expressing IGF1 WT and a FGF receptor variant resulted in increased viable cell density as compared to the controls. Overall, this data showed that cell lines engineered to express IGF1 WT and a FGF receptor variant significantly increased the viable cell density as compared to controls.

### 8.12. Example 9: Cells Engineered for Complete Growth Factor-Independence Using FGF2 Receptors

In this example chicken cells were engineered to assess whether expression of an FGF2 receptor alone was sufficient to enable growth-factor independence (no requirement for growth factors in culture media).

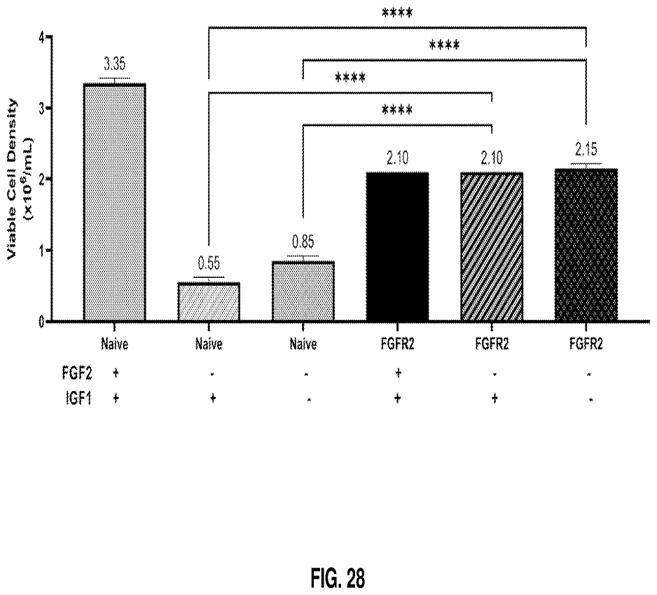

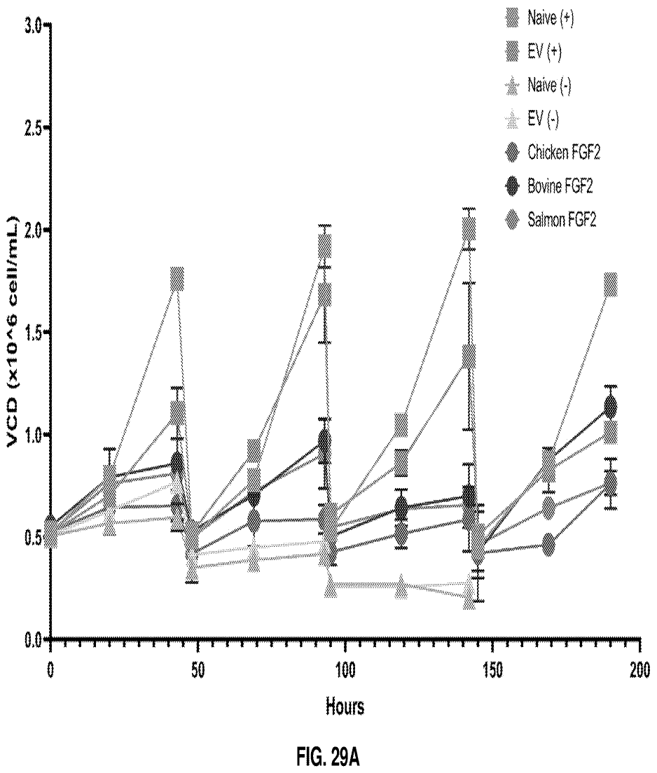

Chicken cells were engineered to expressed FGFR2 and were grown in suspension in a 3 passage proliferation study on a 2-day passaging cadence and initial seeding density of 0.5E6 cells/mL (N=2). Cells were grown in media with or without IGF1 (10 ug/L) and FGF2 (100 ug/L) (see FIG. 28). Naïve (no transduced chicken cells) were used as controls. FIG. 28 shows viable cell density at day 2 of passage 3. One way ANOVA performed compared to Naive controls, Not significant (n.s.), p<0.05 (*), p<0.001(**), p<0.000 (***)p<0.0001 (****).

As shown in FIG. 28, engineering the chicken cells to express FGFR alone was, to Applicant's surprise, sufficient to enable both FGF2 and IGF independent proliferation, albeit at slight lower levels than the naïve chicken cells grown in the presence of FGF2 and IGF1.

### 8.13. Example 10: Cells Engineered for Complete Growth Factor-Independence Using FGF2

In this example chicken cells were engineered to assess whether expression of an FGF2 variant alone was sufficient to enable growth-factor independence (no requirement for growth factors in culture media).

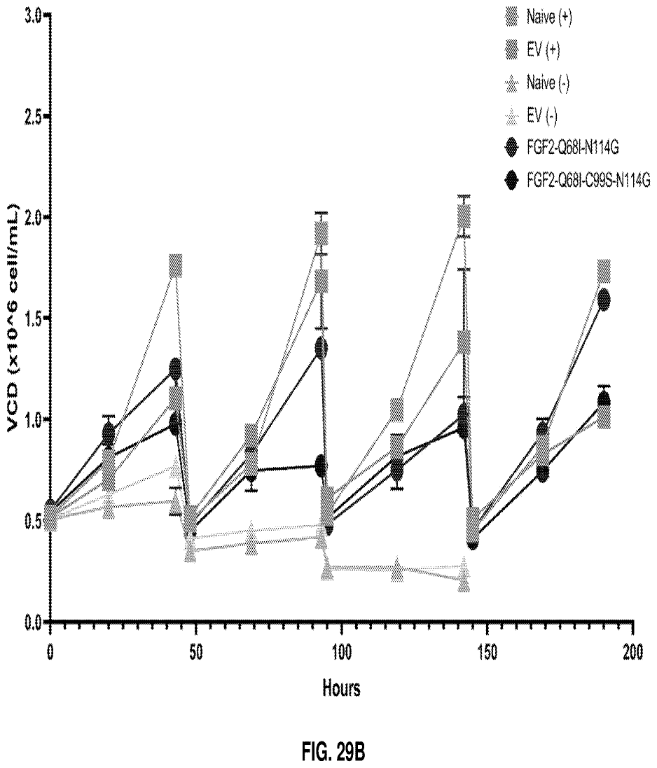

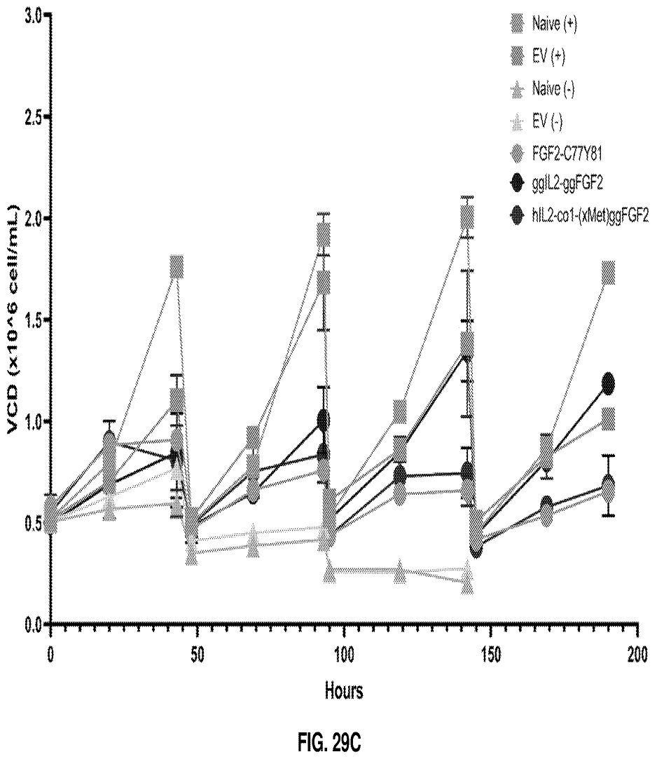

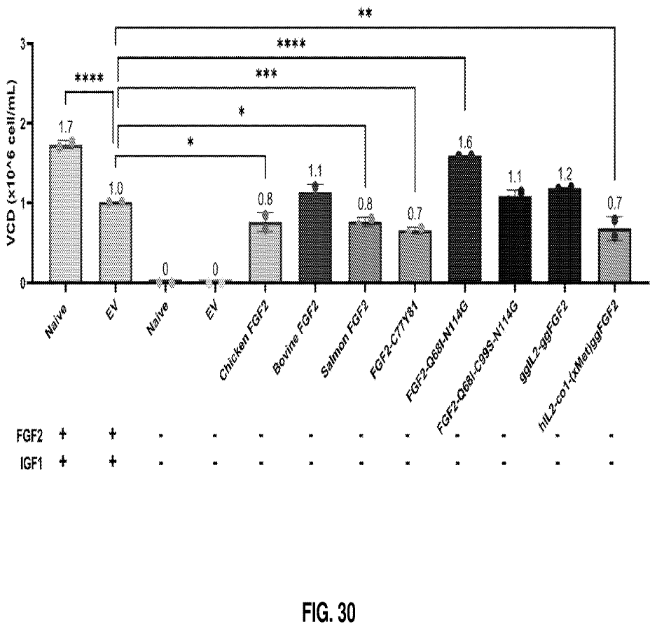

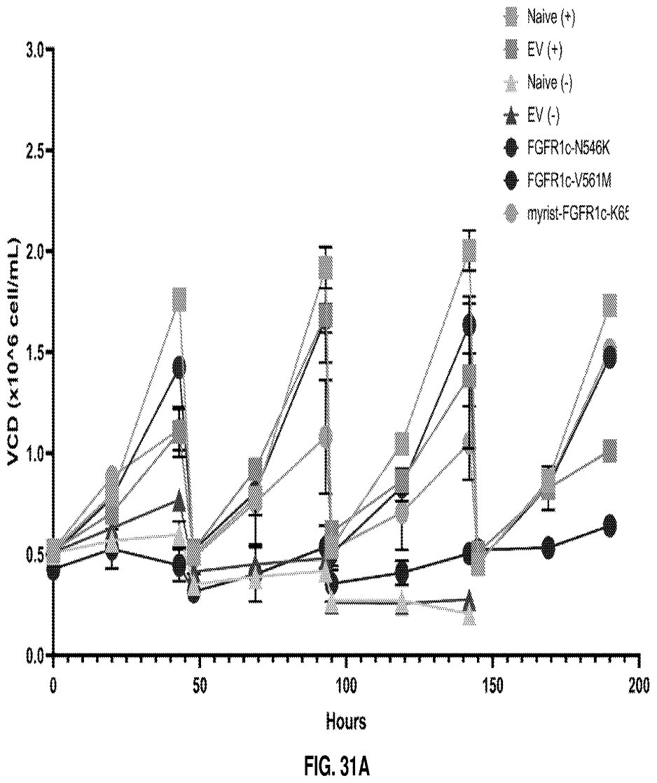

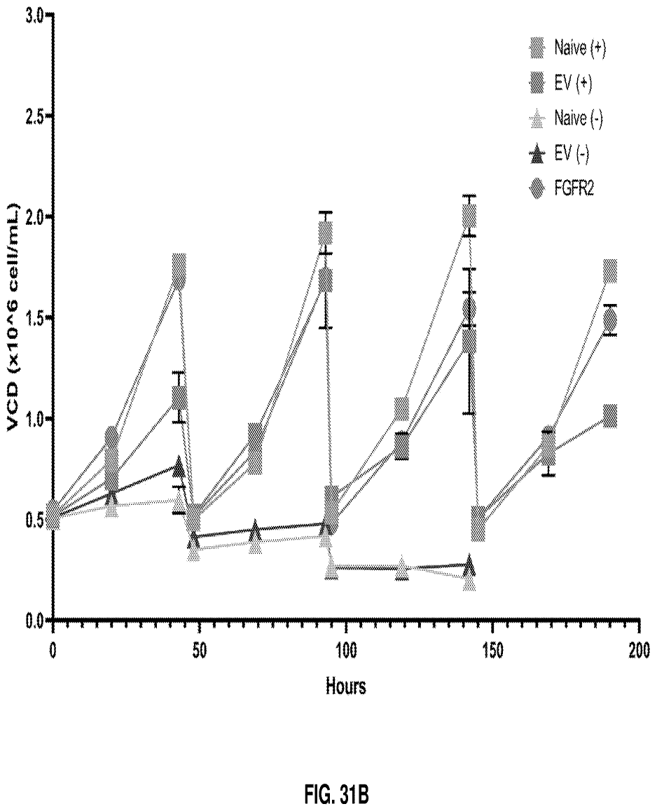

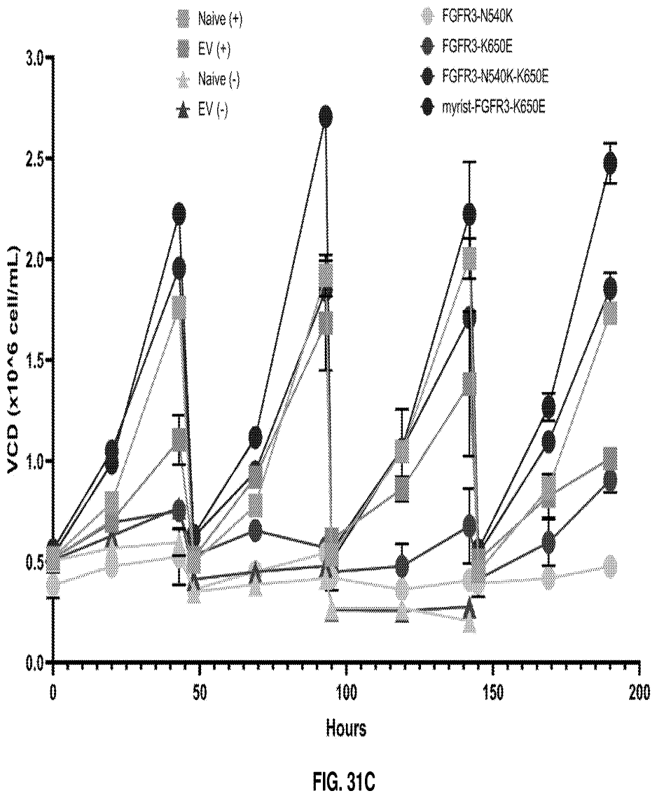

Chicken cells were engineered to express: FGF2 (wild type (FIG. 31A): chicken FGF2, bovine FGF2, salmon FGF2; heat stability point mutations (FIG. 31B): chicken FGF2-Q68I-N114G or chicken FGF2-Q68I-C99S-N114G; and FGF2 with a secretion signal or variant to preserve amino acid for non-canonical secretion (FIG. 31C): chicken FGF2-C77Y8a, ggIL2-ggFGF2, and hIL2-col-(xMet)ggFGF2). These cells were grown in growth factor-free media (ACF media) over 4 passages on 2 day passaging cadence with initial seeding density of 0.5E6 cells/mL (N=2). Naive and empty vector (EV) controls were grown in growth factor containing media (10 μg/L IGF1 and 100 μg/L FGF2) or growth factor-free media (0 μg/L IGF1 and 0 μg/L FGF2). FIGS. 29A-29C shows viable cell density measurements taken over the course of about 200 hours in culture. Notably, controls grown in growth factor free media died by passage 3 (see FIGS. 29A-29C). FIG. 30 shows summary of VCD data from FIGS. 29A-29C at passage 4 day 2 (N=2). One way ANOVA was performed compared to empty vector control in growth factor containing media: not significant (n.s.), p<0.05 (*), p<0.001(**), p<0.0001(***), and p<0.0001 (****).

Overall, this data showed that chicken cells engineered to express FGF2 from different species (chicken, bovine, and salmon) can be used to maintain VCD (viable cell density) over 200 hours in culture as compared to controls, and in some instances, increase VCD compared to controls (FIG. 29A and FIG. 30). This data also showed that chicken cells engineered to express FGF2 variants that have amino acid substitutions that increase heat stability can be used to maintain VCD (viable cell density) over 200 hours in cultures as compared to controls, and in some instances, increase VCD compared to controls (FIG. 29B and FIG. 30). Lastly, this data showed that chicken cells engineered to express FGF2 designed with secretion signals or variants to preserve non-canonical secretion could be used to maintain VCD (viable cell density) over 200 hours in cultures as compared to controls, and in some instances, increase VCD compared to controls (FIG. 29C and FIG. 30).

### 8.14. Example 11: Cells Engineered for Complete Growth Factor-Independence Using FGF2 Receptors Alone

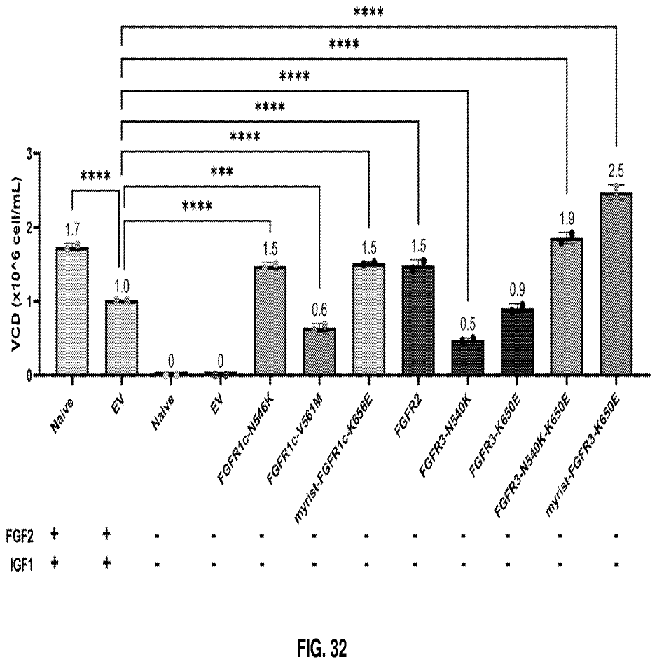

In this example chicken cells were engineered to assess whether expression of an FGF2 receptor variant alone was sufficient to enable growth-factor independence (no requirement for growth factors in culture media) (see FIGS. 31A-31C and FIG. 32).

In a first set of experiments, chicken cells were engineered to express an FGFR1c-N546K, FGF1Rc-V561M, and myrist-FGF1Rc-K656E and were grown in growth factor-free media over 4 passages on 2 day passaging cadence with initial seeding density of 0.5E6 cells/mL (N=2) (see FIG. 31A). Naive and empty vector (EV) controls were grown in growth factor containing media (10 μg/L IGF1 and 100 μg/L FGF2) or growth factor-free media (0 μg/L IGF1 and 0 μg/L FGF2).

FIG. 31A and FIG. 32 show viable cell density (VCD) for chicken cells engineered to express an FGFR1c-N546K, FGF1Rc-V561M, and myrist-FGF1Rc-K656E over 4 passages. Controls grown in growth factor free media died by passage 3 (see FIG. 31A). FIG. 31A shows daily viable cell densities over 4 passages in ACF media. Overall, this data showed that chicken cells engineered to express these FGFR1c variants can be used to maintain VCD (viable cell density) over 200 hours similar to controls, and in some instances, increase VCD compared to controls.

In a second set of experiments, chicken cells were engineered to express FGFR2 and were grown in growth factor-free media over 4 passages on 2 day passaging cadence with initial seeding density of 0.5E6 cells/mL (N=2) (see FIG. 31B). Naive and empty vector (EV) controls were grown in growth factor containing media (10 μg/L IGF1 and 100 μg/L FGF2) or growth factor-free media (0 μg/L IGF1 and 0 μg/L FGF2).

FIG. 31B and FIG. 32 show viable cell density (VCD) for chicken cells engineered to express an FGFR2 over 4 passages. Controls grown in growth factor free media died by passage 3 (see FIG. 31B). Overall, this data showed that chicken cells engineered to express FGFR2 can be used to maintain VCD (viable cell density) over 200 hours in culture similar to controls, and in some instances, increase VCD compared to controls.

In a third set of experiments, chicken cells were engineered to express FGFR3-N540K, FGFR3-K650E, FGFR3-N540K-K560E, or myrist-FGFR3-K650E and were grown in growth factor-free media over 4 passages on 2 day passaging cadence with initial seeding density of 0.5E6 cells/mL (N=2) (see FIG. 31C). Naive and empty vector (EV) controls were grown in growth factor containing media (10 μg/L IGF1 and 100 μg/L FGF2) or growth factor-free media (0 μg/L IGF1 and 0 μg/L FGF2).

FIG. 31C and FIG. 32 show viable cell density (VCD) for chicken cells engineered to express these FGFR3 variants over 4 passages. Controls grown in growth factor free media died by passage 3 (see FIG. 31C). Overall, this data showed that chicken cells engineered to express FGFR3 (or variants thereof) can be used to maintain VCD (viable cell density) over 200 hours in culture similar to controls, and in some instances, increase VCD compared to controls.

To Applicant's surprise, expression of FGF2R receptors alone enabled the serum free proliferation of chicken cells without the need for IGF1 and FGF2 exogenous supplementation. Cell growth is typically limited in the absence of IGF1 and is needed to achieve robust cell proliferation and high cell densities.

### 8.15. Example 12: Cells Engineered to Express PDGF (or Variants Thereof) or PDGF Receptor (or Variants Thereof) Enabled Tissue Formation in Growth Factor-Free Media

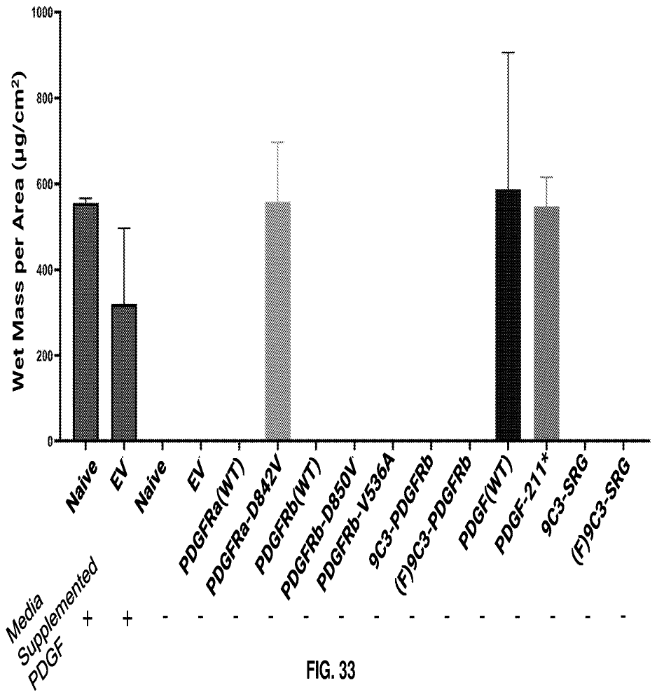

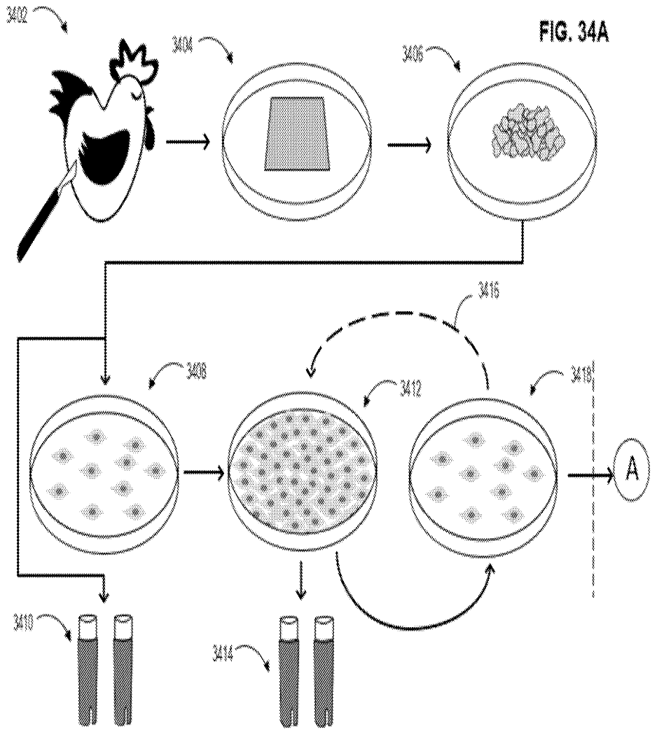

This experiment was designed to assess how expressing PDGF (or PDGF variants) or PDGF receptor (or PDGF receptor variants) enabled tissue formation (as measured by Wet Mass per Area (pg/cm2) (see FIG. 33)).

In particular, chicken cells were transduced with either an FC550A empty vector (“EV”) control or a Phi31 vector containing PDGFRa-D842V, PDGF(WT) or PDGF-211*. Controls included: cell lines not transfected (“Naïve”) and the EV control. Engineered cells and controls were cultured in roller bottles over 14 day period in serum free media. Controls were grown with or without 50 μg/L PDGF (N=4). Tissue was harvested after 14 days culture and weighed for wet mass and normalized to growth area.

FIG. 33 shows chicken cells engineered to express PDGFRa-D842V, PDGF(WT) or PDGF-211* formed tissue without media supplemented growth factors and was comparable to positive controls. Overall, this data showed that chicken cells engineered to express PDGF (or a variant thereof) or a PDGF receptor (or a variant thereof) formed tissue when grown in media not supplemented with growth factors and did so in comparable volume to positive controls. Therefore, these cells lines are ideal for use in cell-based meat products and cell-based food products suitable for consumption because they can endure the requisite culture conditions (i.e., extensive passaging) needed to make these products all while reducing the costs associated with using the vast amounts of exogenous growth factors traditionally needed to make these products.

### 8.16. Example 13: Methods for Producing Cell-Based Meat Suitable for Consumption

The manufacturing of cultured muscle cells suitable for consumption, in one exemplary protocol, can comprise:

In exemplary first step, cells are engineered to express the coding sequence of a growth factor ligand (e.g., FGF2, IGF1, PDGF, or a combination thereof), a growth factor receptor or activated growth factor receptor (e.g., a FGFR, an IGF1R, or an PDGFR), where the cells are from a livestock, poultry, game, or an aquatic animal species. Engineered cells are adapted to suspension culture and cultured in a cultivation infrastructure (e.g., any of the cultivation infrastructures described herein), seeded onto a substrate consisting of peptide-coated tissue-culture treated plastic in a standard growth medium at a density of 7.5×10³ cells/cm² and cultured at 37° C. under 5% CO2 atmospheric conditions. As cultures approach 80% confluence, cells are enzymatically dissociated and the cells are expanded. This process is repeated until the total number of cells harvested following dissociation exceeds about 1.0×10⁸ cells, or more. The engineered cells are cryopreserved and stored in a cryopreserved cell bank. Cells harvested in quantities equal to or exceeding 1.0×10⁸ cells.

In an exemplary second step, the cryopreserved cells are seeded and cultivated in a cultivation infrastructure. In accordance with the cultivation scale desired, one or more vials from the master cell bank is rapidly thawed to room temperature. The cryopreservation medium is removed from the cells by a 5-minute, 300×g centrifugation step. Cells are suspended in standard growth medium and seeded onto a gelatin-coated cultivation substrate in standard growth medium as before, except that, on the final passage prior to harvest, the cells are permitted to proliferate to 100% confluence on the cell culture substrate. The cells are adapted for suspension culture in growth media to facilitate expansion of the cells. Following expansion, the cells are adapted to adherent culture in a cultivation infrastructure. The growth medium is exchanged for differentiation medium, thereby inducing myogenic differentiation where the differentiated cells form myocytes and multinucleated myotubes; and the myocytes and myotubes are cultured to generate skeletal muscle fibers.

In an exemplary fourth step, the engineered cells are harvested for dietary consumption. After the cells have proliferated to confluence, the culture medium is removed, and the adherent cell cultures are rinsed with phosphate buffered saline. Next, the confluent biomass of adherent cells are mechanically, fluidically, enzymatically, or metabolically dissociated from the substrate by means of a scraping device, pressurized fluid, or a harvest media. The dissociated biomass is collected into centrifuge tubes, pelleted to remove excess liquid, and processed for food product preparation.

### 8.17. Example 14: Procurement of Engineered Cells and Growth of Cells into a Cell Mass

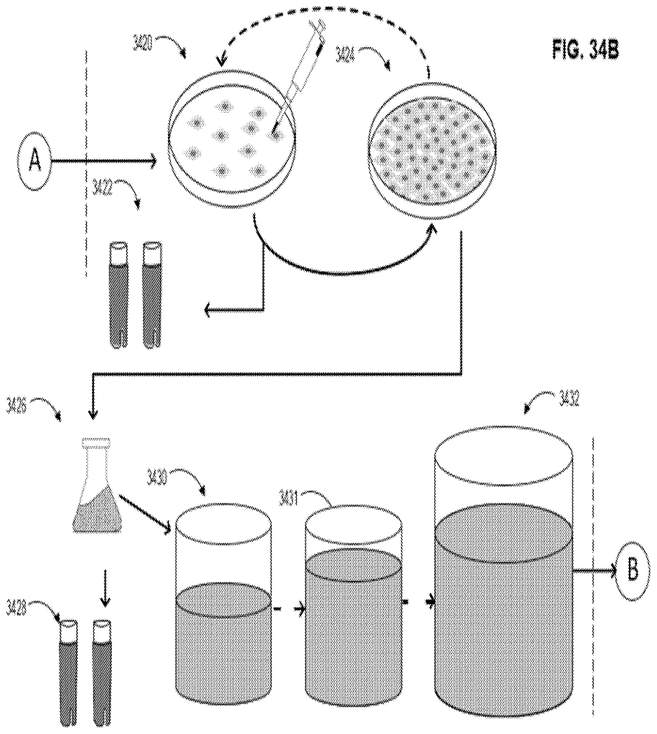

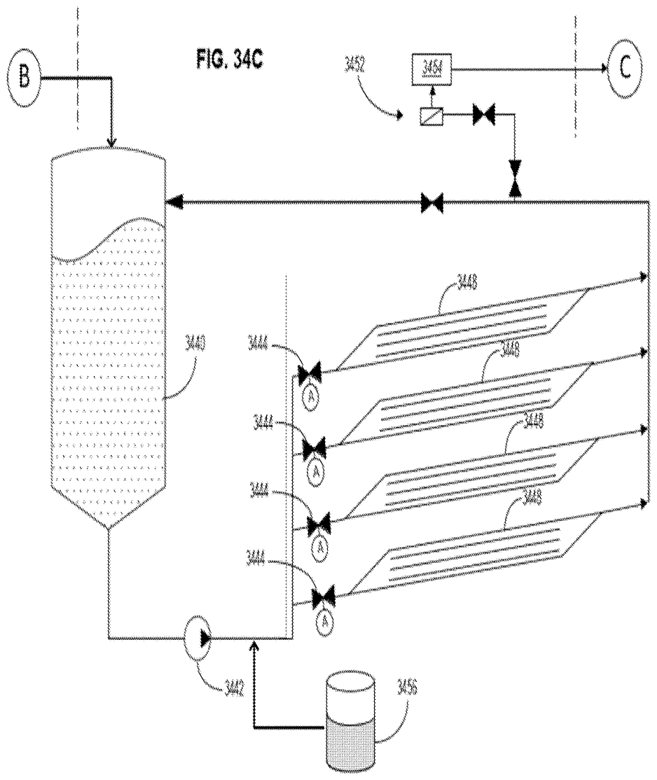

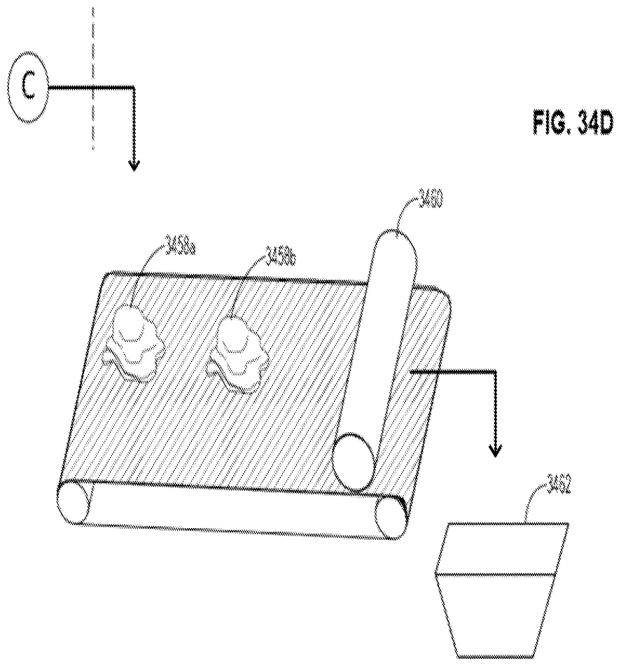

FIGS. 34A-34D and the following accompanying paragraphs describe procurement of cells and growth of cells into a cell mass in accordance with one or more embodiments. Generally, FIGS. 34A-34D illustrate a process of collecting cells from an animal, growing cells in a favorable environment, banking successful cells, and collecting cells into a cell mass followed by de-wetting and/or other treatments.

As illustrated by step 3402 in FIG. 34A, tissue is collected from a living animal via biopsy. In particular, stem cells, mesenchymal progeny, ectoderm lineage, and/or endoderm lineages can be isolated from the removed tissue. In some implementations of the present disclosure, tissue, such as fat and others, are processed to isolate stem cells, mesenchymal, ectoderm, and/or endoderm progeny or lineage cells. As illustrated, tissue 3404 is removed from an animal. In some examples, the tissue 3404 is removed from a living animal by taking a skin sample from the living animal. For instance, skin or muscle samples may be taken from a chicken, cow, fish, shellfish or another animal.

Cells may be extracted from the tissue 3404 that was removed from the animal. More specifically, the tissue 3404 is broken down by enzymatic and/or mechanical means. To illustrate, FIG. 34A includes digested tissue 3406 that comprises the cells to be grown in cultivation.

Cells in the digested tissue 3406 may be proliferated under appropriate conditions to begin a primary culture. As illustrated in FIG. 34A, cells 3408 from the digested tissue 3406 are spread on a surface or substrate and proliferated until they reach confluence. As shown in FIG. 34A, in some cases, cells 3412 have reached confluence when they start contacting other cells in the vessel, and/or have occupied all the available surface or substrate.

In some examples, cells are stored and frozen (i.e., banked) at different steps along the cell culture process. Cryopreservation generally comprises freezing cells for preservation and long-term storage. In some implementations, tissue and/or cells are removed from a surface or substrate, centrifuged to remove moisture content, and treated with a protective agent for cryopreservation. For example, as part of cryopreservation, tissues and cells are stored at temperatures at or below −80 C. The protective agent may comprise dimethyl sulfoxide (DMSO) or glycerol.

Cells stored through cryopreservation may be used to replenish working cell stock. For instance, while a portion of the digested tissue 3406 is used as the cells 3408 spread on a surface or substrate, the remaining or excess digested tissue 3406 is transferred to cryovials 3410 for storage. Furthermore, the cells 3412 may be banked once reaching confluence and stored in cryovials 3414.

Once the cells 3412 have reached confluence, or just before the cells 3412 have reached confluence (e.g., occupation of about 80% of the substrate), the disclosed process comprises a series of cell passage steps. During cell passage, the cells 3412 are divided into one or more new culture vessels for continued proliferation. To illustrate, the cells 3412 may be diluted or spread on one or more surfaces or substrates to form the cells 3418. The cells 3418 are then grown 3416 to confluence, or just before confluence.

The cycle of dividing the cells 3412 into the cells 3418 for continued proliferation in new culture vessels may be repeated for a determined number of cycles. Typically, cell lines derived from primary cultures have a finite life span. Passaging the cells allows cells with the highest growth capacity to predominate. In one example, cells are passaged for five cycles to meet a desired genotypic and phenotypic uniformity in the cell population.

In some implementations, the disclosed method comprises immortalizing cells that have been grown and passaged for the determined number of cycles. For instance, the cells 3418 may be immortalized. As shown in FIG. 34B, cells 3420 have demonstrated a preferred growth capacity to proceed to immortalization. To achieve immortalization, the disclosed process transfects the cells 3420 with genes of interest. In one example telomerase reverse transcriptase (TERT) is introduced to the cells 3420. In some embodiments, the cells may be subjected to a selection process as known by those skilled in the art. The cells 3420 may then be passaged for a predetermined set of passaging cycles. In one example passaging cycle, the cells 3420 are grown to (or near) confluence 3424, then they are reseeded in new growth vessels, preserved in vials 3422, or some combination of both. The disclosed process may include any number of passaging cycles to ensure that the cells have reached immortality (e.g., can passage 60+ times without senescing), a target growth capacity, and/or a target quantity for banking. For example, cells may be passaged until they have reached a passage level of 100 (e.g., have been passaged for 100 passaging cycles). In another example, cells are passaged until they reach a population doubling level of 100.

Cells that have reached immortality or a target growth capacity by living through a target passage level may be adapted to suspension culture. In one example, a suspension culture media and agitation of cells in this suspension environment help cells to adapt and start proliferating in the new growth environment. The cells adapted to suspension 3426 may be stored in cryovials 3428 for cryopreservation and banking. Cells in suspension 3426 will begin to proliferate and the process begins a series of dilute and expand steps.

During dilution and expansion, cells are moved from growth vessels into newer, and progressively larger, growth vessels. For example, cells in suspension 3426 may begin in a single tube. The cells will proliferate and increase in cellular density. Once the cells have reached a target cell number (i.e., viable cell density (VCD) at desired volume), they are diluted and moved to a larger growth vessel. Optionally, the cells are banked in cryovials throughout expansion. For example, once cells in suspension reach a maximum VCD, the cells may begin to leave exponential growth due to overcrowding. After reaching a target density, the suspension cells may be transferred to a larger vessel 3430 and diluted with additional media. The dilute-and-expand steps are repeated using progressively larger vessels (e.g., the vessel 3431 and the vessel 3432) and/or progressive dilution until the cells reach a production-ready volume. For example, cells may be production ready at about a 1,000-100,000 liter scale at 5 million cells per mL. The cells may be banked in cryovials at any of the dilution and expansion cycles.

As part of preparing cells to form cell-based-meat products, the disclosed process comprises growing the cells as an adherent culture. Generally, cells that are grown attached to a substrate form a texture that more closely resembles tissue found in conventional meat. Thus, the cells may be transferred from growth in suspension to growth in an adherent reactor. For example, the cells grown in suspension in the vessel 3432 may be transferred to growth on a substrate. FIG. 34C illustrates a bioreactor system comprising a plurality of adherent bioreactors 3448 connecting in parallel to a media vessel 3440. The media vessel 3440 holds the cells grown in suspension media. In some implementations, cells from the vessel 3432 are transferred directly to a cell culture media (or just “media”) vessel 3440. In one example, the media vessel 3440 comprises the vessel 3432. The adherent bioreactors 3448 may comprise pipe-based bioreactors. As shown, a plurality of valves 3444 is secured to the plurality of adherent bioreactors 3448 to enable individual use and access of each of the adherent bioreactors 3448. For instance, to limit flow to only a first bioreactor of the plurality of adherent bioreactors 3448, the valve 3444 of the first bioreactor is opened while the remaining valves 3444 are closed. Furthermore, the bioreactor system can include a directional valve 3442 for changing between flow directions.

In some implementations, and as illustrated in FIG. 34C, cells (e.g., adherent cells or suspension adapted cells) are prepared by flowing cells suspended in media (e.g., cell culture media) across substrates in the plurality of adherent bioreactors 3448. More particularly, cells from the media vessel 3440 may contact or land on the substrates in the plurality of adherent bioreactors 3448. Cells and media that flowed through the adherent bioreactors 3448 are cycled back to the media vessel 3440. The media and cells can be cycled through the adherent bioreactors 3448 until a target adherent cell density is reached. For instance, in some implementations, the disclosed method comprises measuring a cell density of outflow from the adherent bioreactors 3448 to infer an adherent cell density.

The cells grow into adherent tissue within the adherent bioreactors 3448. Once they have grown to a target density, either according to a learned timing or according to a measured fluctuation in cell metabolism of components such as glucose and oxygen, then the adherent tissue is ready for removal. The removal process of the disclosed method uses a high-pressure flow to shear the adherent tissue off the substrate surfaces. In one example, wash buffer from a wash tank 3456 is flowed across the substrates in the adherent bioreactors 3448. The wash buffer and cell mixture are flowed through a filter 3452 where the cells are collected into one or more cell masses 3454.

The cell masses 3454 may be further processed to adjust moisture content. FIG. 34D illustrates an example apparatus for reducing moisture content in the cells. In particular, FIG. 34D illustrates a pressure apparatus 3460 that compresses the cell masses 3458a and 3458b. While FIG. 34D illustrates a mechanical method for adjusting moisture content of the cell masses 3458a and 3458b, other methods may be used to adjust moisture content. For example, the cell masses 3458a and 3458b may be mixed with a drying agent, vacuum dried, centrifuged, or otherwise dried. A moisture-adjusted-cell mass may be transferred to a container 3462 for additional processing. For example, the cell mass 3458a or 3458b may be removed from the container 3462 to be formed into a cell-based-meat product.

9. SEQUENCE APPENDIX

| SEQ | | |
|---|---|---|
| ID | | |
| NO: | Description | Sequence |
| 1 | ggFGF2-Wild | MAAGAAGSITTLPALPDDGGGGAFPPGHFKDPKRLYCKNGGFFLRINPDGRVD |
| | Type (chicken) | GVREKSDPHIKLQLQAEERGVVSIKGVSANRFLAMKEDGRLLALKCATEECFFF |
| | | ERLESNNYNTYRSRKYSDWYVALKRTGQYKPGPKTGPGQKAILFLPMSAKS* |
| 2 | ggFGF2STAB | MAAGAAGSITTLPALPDDGGGGAFPPGHFKDPKRLYCKNGGFFLLINPDGRVD |
| | (Thermal | GTREKSDPFIKLQLQAEERGVVSIKGVSANRFLAMKEDGRLYALKYATEECFFF |
| | Stabilized) | ERLEENNYNTYRSRKYSDWYVALKRTGQYKPGPKTGPGQKAILFLPMSAKS* |
| 3 | BM40sp- | MRAWIFFLLCLAGRALAMAAGAAGSITTLPALPDDGGGGAFPPGHFKDPKRLY |
| | ggFGF2 | CKNGGFFLRINPDGRVDGVREKSDPHIKLQLQAEERGVVSIKGVSANRFLAMK |
| | | EDGRLLALKCATEECFFFERLESNNYNTYRSRKYSDWYVALKRTGQYKPGPKT |
| | | GPGQKAILFLPMSAKS* |
| 4 | ggIL2-ggFGF2 | MMCKVLIFGCISVAMLMTTAYMAAGAAGSITTLPALPDDGGGGAFPPGHFKDP |
| | | KRLYCKNGGFFLRINPDGRVDGVREKSDPHIKLQLQAEERGVVSIKGVSANRFL |
| | | AMKEDGRLLALKCATEECFFFERLESNNYNTYRSRKYSDWYVALKRTGQYKP |
| | | GPKTGPGQKAILFLPMSAKS* |
| 5 | Gluc-ggFGF2 | MGVKVLFALICIAVAEAMAAGAAGSITTLPALPDDGGGGAFPPGHFKDPKRLY |
| | | CKNGGFFLRINPDGRVDGVREKSDPHIKLQLQAEERGVVSIKGVSANRFLAMK |
| | | EDGRLLALKCATEECFFFERLESNNYNTYRSRKYSDWYVALKRTGQYKPGPKT |
| | | GPGQKAILFLPMSAKS* |
| 6 | cohIL2- | MYRMQLLSCIALSLALVTNSMAAGAAGSITTLPALPDDGGGGAFPPGHFKDPK |
| | ggFGF2 | RLYCKNGGFFLRINPDGRVDGVREKSDPHIKLQLQAEERGVVSIKGVSANRFLA |
| | | MKEDGRLLALKCATEECFFFERLESNNYNTYRSRKYSDWYVALKRTGQYKPG |
| | | PKTGPGQKAILFLPMSAKS* |
| 7 | cohIL2co1- | MRMQLLLLIALSLALVTNSMAAGAAGSITTLPALPDDGGGGAFPPGHFKDPKR |
| | ggFGF2 | LYCKNGGFFLRINPDGRVDGVREKSDPHIKLQLQAEERGVVSIKGVSANRFLA |
| | | MKEDGRLLALKCATEECFFFERLESNNYNTYRSRKYSDWYVALKRTGQYKPG |
| | | PKTGPGQKAILFLPMSAKS* |
| 8 | cohIL2co2- | MRRMQLLLLIALSLALVTNSMAAGAAGSITTLPALPDDGGGGAFPPGHFKDPK |
| | ggFGF2 | RLYCKNGGFFLRINPDGRVDGVREKSDPHIKLQLQAEERGVVSIKGVSANRFLA |
| | | MKEDGRLLALKCATEECFFFERLESNNYNTYRSRKYSDWYVALKRTGQYKPG |
| | | PKTGPGQKAILFLPMSAKS* |
| 9 | ggIL2- | MMCKVLIFGCISVAMLMTTAYMAAGAAGSITTLPALPDDGGGGAFPPGHFKDP |
| | FGF2STAB | KRLYCKNGGFFLLINPDGRVDGTREKSDPFIKLQLQAEERGVVSIKGVSANRFL |
| | | AMKEDGRLYALKYATEECFFFERLEENNYNTYRSRKYSDWYVALKRTGQYKP |
| | | GPKTGPGQKAILFLPMSAKS* |
| 10 | hIL2- | MYRMQLLSCIALSLALVTNSMAAGAAGSITTLPALPDDGGGGAFPPGHFKDPK |
| | ggFGF2STAB | RLYCKNGGFFLLINPDGRVDGTREKSDPFIKLQLQAEERGVVSIKGVSANRFLA |
| | | MKEDGRLYALKYATEECFFFERLEENNYNTYRSRKYSDWYVALKRTGQYKPG |
| | | PKTGPGQKAILFLPMSAKS* |
| 11 | ggIL6-ggFGF2 | MNFTEGCEATGRRPGSAGSRRRRAPRPGPVALLPLLLPLLLPPAAAVPLPMAAG |
| | | AAGSITTLPALPDDGGGGAFPPGHFKDPKRLYCKNGGFFLRINPDGRVDGVREK |
| | | SDPHIKLQLQAEERGVVSIKGVSANRFLAMKEDGRLLALKCATEECFFFERLES |
| | | NNYNTYRSRKYSDWYVALKRTGQYKPGPKTGPGQKAILFLPMSAKS* |
| 12 | hIL2-ggFGF2 | MNSFSTSAFGPVAFSLGLLLVLPAAFPAPMAAGAAGSITTLPALPDDGGGGAFP |
| | | PGHFKDPKRLYCKNGGFFLRINPDGRVDGVREKSDPHIKLQLQAEERGVVSIKG |
| | | VSANRFLAMKEDGRLLALKCATEECFFFERLESNNYNTYRSRKYSDWYVALK |
| | | RTGQYKPGPKTGPGQKAILFLPMSAKS* |
| 13 | hIL2-ggFGF2 | MYRMQLLSCIALSLALVTNSMAAGAAGSITTLPALPDDGGGGAFPPGHFKDPK |
| | | RLYCKNGGFFLLINPDGRVDGTREKSDPFIKLQLQAEERGVVSIKGVSANRFLA |
| | | MKEDGRLYALKYATEECFFFERLEENNYNTYRSRKYSDWYVALKRTGQYKPG |
| | | PKTGPGQKAILFLPMSAKS* |
| 14 | hIFNa2- | MALTFALLVALLVLSCKSSCSVGMVSKMAAGAAGSITTLPALPDDGGGGAFPP |
| | ggFGF2 | GHFKDPKRLYCKNGGFFLRINPDGRVDGVREKSDPHIKLQLQAEERGVVSIKG |
| | | VSANRFLAMKEDGRLLALKCATEECFFFERLESNNYNTYRSRKYSDWYVALK |
| | | RTGQYKPGPKTGPGQKAILFLPMSAKS* |
| 15 | secrecon- | MWWRLWWLLLLLLLLWPMVWAMAAGAAGSITTLPALPDDGGGGAFPPGHF |
| | ggFGF2 | KDPKRLYCKNGGFFLRINPDGRVDGVREKSDPHIKLQLQAEERGVVSIKGVSA |
| | | NRFLAMKEDGRLLALKCATEECFFFERLESNNYNTYRSRKYSDWYVALKRTG |
| | | QYKPGPKTGPGQKAILFLPMSAKS* |
| 16 | ggIGF1-WT | MEKINSLSTQLVKCCFCDFLKVKMHTVSYIHFFYLGLCLLTLTSSAAAGPETLC |
| | | GAELVDALQFVCGDRGFYFSKPTGYGSSSRRLHHKGIVDECCFQSCDLRRLEM |
| | | YCAPIKPPKSARSVRAQRHTDMPKAQKEVHLKNTSRGNTGNRNYRM* |
| 17 | ggIGF1- | MGPETLCGAELVDALQFVCGDRGFYFSKPTGYGSSSRRLHHKGIVDECCFQSC |
| | truncated | DLRRLEMYCAPIKPPKSA* |
| 18 | ggPDGFb | MCPQPARLEPGMNFGVVFAVILSLPLARLEGDPIPEDIYEILGGSSVRSISDLQRA |
| | | LRIDSVEEDSSSLDLNATQPSQNHVSLSRERRSLDALAAAEPAVLAECKTRTVV |
| | | FEISRDMVDSTNANFVVWPPCVEVQRCSGCCNNRNVQCRPMQIRVRHVQVNK |
| | | IEFFQRKPIFKKVIVPLEDHVQCRCEVVSRPPPRSNRPASREQRRFSPSFTTAAISQ |
| | | RKRVRRPPAQKRKHKKYKHVNDKKVLKEILIA* |
| 19 | ggPDGFb- | MCPQPARLEPGMNFGVVFAVILSLPLARLEGDPIPEDIYEILGGSSVRSISDLQRA |
| | 211STOP | LRIDSVEEDSSSLDLNATQPSQNHVSLSRERRSLDALAAAEPAVLAECKTRTVV |
| | | FEISRDMVDSTNANFVVWPPCVEVQRCSGCCNNRNVQCRPMQIRVRHVQVNK |
| | | IEFFQRKPIFKKVIVPLEDHVQCRCEVVSRPPPRSNRPASREQRRFSPSFTTAAISQ* |
| 20 | F-9C-SRG | MADYKDDDDKKGGIIVAILLLIVMLAIEILLLITLIIAVTSGGSG* |
| 21 | 9C-SRG | MAKGGIIVAILLLIVMLAIEILLLITLIIAVTSGGSG* |
| 22 | IL2 (human) | MYRMQLLSCIALSLALVTNS |
| | (“hil2” or | |
| | “hIL2”) | |
| | Signal peptide | |
| 23 | IL2-CO1 | MRMQLLLLIALSLALVTNS |
| | (human) | |
| | (“hil2col1”) | |
| | Signal peptide | |
| 24 | IL2-CO2 | MRRMQLLLLIALSLALVTNS |
| | (human) | |
| | (“hil2col2”) | |
| | Signal | |
| | peptide | |
| 25 | IL2 (chicken) | MMCKVLIFGCISVAMLMTTAY |
| | (“ggil2” or | |
| | “ggIL2”) | |
| | Signal peptide | |
| 26 | IFNa (human) | MALTFALLVALLVLSCKSSCSVGMVSK |
| | (“hIFNA”) | |
| | Signal peptide | |
| 27 | IL6 (human) | MNSFSTSAFGPVAFSLGLLLVLPAAFPAP |
| | (“hil6”) Signal | |
| | peptide | |
| 28 | IL6 (chicken) | MNFTEGCEATGRRPGSAGSRRRRAPRPGPVALLPLLLPLLLPPAAAVPLP |
| | (“ggil6”) | |
| | Signal peptide | |
| 29 | Gaussia | MGVKVLFALICIAVAEA |
| | Luciferase | |
| | (“GLuc”) | |
| | Signal peptide | |
| 30 | Secrecon | MWWRLWWLLLLLLLLWPMVWA |
| | Signal peptide | |
| 31 | BM40 signal | MRAWIFFLLCLAGRALA |
| | peptide | |
| | Signal peptide | |
| 32 | FGFR1-WT | MFTWRCLILWAVLVTATLSAARPAPTLPDQALPKANIEVESHSAHPGDLLQLR |
| | | CRLRDDVQSINWVRDGVQLPENNRTRITGEEVEVRDAVPEDSGLYACMTNSPS |
| | | GSETTYFSVNVSDALPSAEDDDDEDDSSSEEKEADNTKPNQAVAPYWTYPEKM |
| | | EKKLHAVPAAKTVKFKCPSGGTPNPTLRWLKNGKEFKPDHRIGGYKVRYATW |
| | | SIIMDSVVPSDKGNYTCIVENKYGSINHTYQLDVVERSPHRPILQAGLPANKTV |
| | | ALGSNVEFVCKVYSDPQPHIQWLKHIEVNGSKIGPDNLPYVQILKTAGVNTTDK |
| | | EMEVLHLRNVSFEDAGEYTCLAGNSIGISHHSAWLTVLEATEQSPAMMTSPLY |
| | | LEIIIYCTGAFLISCMVVTVIIYKMKSTTKKTDFNSQLAVHKLAKSIPLRRQVTVS |
| | | ADSSSSMNSGVMLVRPSRLSSSGTPMLAGVSEYELPEDPRWELPRDRLILGKPL |
| | | GEGCFGQVVLAEAIGLDKDKPNRVTKVAVKMLKSDATEKDLSDLISEMEMMK |
| | | MIGKHKNIINLLGACTQDGPLYVIVEYASKGNLREYLQARRPPGMEYCYNPTRI |
| | | PEEQLSFKDLVSCAYQVARGMEYLASKKCIHRDLAARNVLVTEDNVMKIADF |
| | | GLARDIHHIDYYKKTTNGRLPVKWMAPEALFDRIYTHQSDVWSFGVLLWEIFT |
| | | LGGSPYPGVPVEELFKLLKEGHRMDKPSNCTNELYMMMRDCWHAVPSQRPTF |
| | | KQLVEDLDRIVAMTSNQEYLDLSVPLDQYSPGFPATRSSTCSSGEDSVFSHDPLP |
| | | DEPCLPRCPPHSHGALKRH* |
| 33 | FGFR1- | MFTWRCLILWAVLVTATLSAARPAPTLPDQALPKANIEVESHSAHPGDLLQLR |
| | N546K | CRLRDDVQSINWVRDGVQLPENNRTRITGEEVEVRDAVPEDSGLYACMTNSPS |
| | | GSETTYFSVNVSDALPSAEDDDDEDDSSSEEKEADNTKPNQAVAPYWTYPEKM |
| | | EKKLHAVPAAKTVKFKCPSGGTPNPTLRWLKNGKEFKPDHRIGGYKVRYATW |
| | | SIIMDSVVPSDKGNYTCIVENKYGSINHTYQLDVVERSPHRPILQAGLPANKTV |
| | | ALGSNVEFVCKVYSDPQPHIQWLKHIEVNGSKIGPDNLPYVQILKTAGVNTTDK |
| | | EMEVLHLRNVSFEDAGEYTCLAGNSIGISHHSAWLTVLEATEQSPAMMTSPLY |
| | | LEIIIYCTGAFLISCMVVTVIIYKMKSTTKKTDFNSQLAVHKLAKSIPLRRQVTVS |
| | | ADSSSSMNSGVMLVRPSRLSSSGTPMLAGVSEYELPEDPRWELPRDRLILGKPL |
| | | GEGCFGQVVLAEAIGLDKDKPNRVTKVAVKMLKSDATEKDLSDLISEMEMMK |
| | | MIGKHKNIIKLLGACTQDGPLYVIVEYASKGNLREYLQARRPPGMEYCYNPTRI |
| | | PEEQLSFKDLVSCAYQVARGMEYLASKKCIHRDLAARNVLVTEDNVMKIADF |
| | | GLARDIHHIDYYKKTTNGRLPVKWMAPEALFDRIYTHQSDVWSFGVLLWEIFT |
| | | LGGSPYPGVPVEELFKLLKEGHRMDKPSNCTNELYMMMRDCWHAVPSQRPTF |
| | | KQLVEDLDRIVAMTSNQEYLDLSVPLDQYSPGFPATRSSTCSSGEDSVFSHDPLP |
| | | DEPCLPRCPPHSHGALKRH* |
| 34 | FGFR1- | MFTWRCLILWAVLVTATLSAARPAPTLPDQALPKANIEVESHSAHPGDLLQLR |
| | K656E | CRLRDDVQSINWVRDGVQLPENNRTRITGEEVEVRDAVPEDSGLYACMTNSPS |
| | | GSETTYFSVNVSDALPSAEDDDDEDDSSSEEKEADNTKPNQAVAPYWTYPEKM |
| | | EKKLHAVPAAKTVKFKCPSGGTPNPTLRWLKNGKEFKPDHRIGGYKVRYATW |
| | | SIIMDSVVPSDKGNYTCIVENKYGSINHTYQLDVVERSPHRPILQAGLPANKTV |
| | | ALGSNVEFVCKVYSDPQPHIQWLKHIEVNGSKIGPDNLPYVQILKTAGVNTTDK |
| | | EMEVLHLRNVSFEDAGEYTCLAGNSIGISHHSAWLTVLEATEQSPAMMTSPLY |
| | | LEIIIYCTGAFLISCMVVTVIIYKMKSTTKKTDFNSQLAVHKLAKSIPLRRQVTVS |
| | | ADSSSSMNSGVMLVRPSRLSSSGTPMLAGVSEYELPEDPRWELPRDRLILGKPL |
| | | GEGCFGQVVLAEAIGLDKDKPNRVTKVAVKMLKSDATEKDLSDLISEMEMMK |
| | | MIGKHKNIINLLGACTQDGPLYVIVEYASKGNLREYLQARRPPGMEYCYNPTRI |
| | | PEEQLSFKDLVSCAYQVARGMEYLASKKCIHRDLAARNVLVTEDNVMKIADF |
| | | GLARDIHHIDYYKETTNGRLPVKWMAPEALFDRIYTHQSDVWSFGVLLWEIFT |
| | | LGGSPYPGVPVEELFKLLKEGHRMDKPSNCTNELYMMMRDCWHAVPSQRPTF |
| | | KQLVEDLDRIVAMTSNQEYLDLSVPLDQYSPGFPATRSSTCSSGEDSVFSHDPLP |
| | | DEPCLPRCPPHSHGALKRH* |
| 35 | FGFR1- | MFTWRCLILWAVLVTATLSAARPAPTLPDQALPKANIEVESHSAHPGDLLQLR |
| | N546K- | CRLRDDVQSINWVRDGVQLPENNRTRITGEEVEVRDAVPEDSGLYACMTNSPS |
| | K656E-S780A | GSETTYFSVNVSDALPSAEDDDDEDDSSSEEKEADNTKPNQAVAPYWTYPEKM |
| | | EKKLHAVPAAKTVKFKCPSGGTPNPTLRWLKNGKEFKPDHRIGGYKVRYATW |
| | | SIIMDSVVPSDKGNYTCIVENKYGSINHTYQLDVVERSPHRPILQAGLPANKTV |
| | | ALGSNVEFVCKVYSDPQPHIQWLKHIEVNGSKIGPDNLPYVQILKTAGVNTTDK |
| | | EMEVLHLRNVSFEDAGEYTCLAGNSIGISHHSAWLTVLEATEQSPAMMTSPLY |
| | | LEIIIYCTGAFLISCMVVTVIIYKMKSTTKKTDFNSQLAVHKLAKSIPLRRQVTVS |
| | | ADSSSSMNSGVMLVRPSRLSSSGTPMLAGVSEYELPEDPRWELPRDRLILGKPL |
| | | GEGCFGQVVLAEAIGLDKDKPNRVTKVAVKMLKSDATEKDLSDLISEMEMMK |
| | | MIGKHKNIIKLLGACTQDGPLYVIVEYASKGNLREYLQARRPPGMEYCYNPTRI |
| | | PEEQLSFKDLVSCAYQVARGMEYLASKKCIHRDLAARNVLVTEDNVMKIADF |
| | | GLARDIHHIDYYKETTNGRLPVKWMAPEALFDRIYTHQSDVWSFGVLLWEIFT |
| | | LGGSPYPGVPVEELFKLLKEGHRMDKPSNCTNELYMMMRDCWHAVPSQRPTF |
| | | KQLVEDLDRIVAMTSNQEYLDLSVPLDQYAPGFPATRSSTCSSGEDSVFSHDPL |
| | | PDEPCLPRCPPHSHGALKRH* |
| 36 | FGFR1- | MGSSKSKPKDPSQRKMKSTTKKTDFNSQLAVHKLAKSIPLRRQVTVSADSSSS |
| | myrist-K656E | MNSGVMLVRPSRLSSSGTPMLAGVSEYELPEDPRWELPRDRLILGKPLGEGCFG |
| | | QVVLAEAIGLDKDKPNRVTKVAVKMLKSDATEKDLSDLISEMEMMKMIGKHK |
| | | NIINLLGACTQDGPLYVIVEYASKGNLREYLQARRPPGMEYCYNPTRIPEEQLSF |
| | | KDLVSCAYQVARGMEYLASKKCIHRDLAARNVLVTEDNVMKIADFGLARDIH |
| | | HIDYYKETTNGRLPVKWMAPEALFDRIYTHQSDVWSFGVLLWEIFTLGGSPYP |
| | | GVPVEELFKLLKEGHRMDKPSNCTNELYMMMRDCWHAVPSQRPTFKQLVED |
| | | LDRIVAMTSNQEYLDLSVPLDQYSPGFPATRSSTCSSGEDSVFSHDPLPDEPCLP |
| | | RCPPHSHGALKRH* |
| 37 | FGF2-WT | MGLKSTWRYGNGPGTYSKKMVSWDSGCLICLVVVTMAGLSLARPSFNLVVED |
| | | ATLEPEEPPTKYQISQPDVHSALPGEPLELRCQLKDAVMISWTKDGVPLGPDNR |
| | | TVIIGEYLQIKDASPRDSGLYACTAIRTLDSDTLYFIVNVTDALSSGDDEDDNDG |
| | | SEDFVNDSNQMRAPYWTHTDKMEKRLHAVPAANTVKFRCPAMGNPTPTMRW |
| | | LKNGKEFKQEHRIGGYKVRNQHWSLIMESVVPSDKGNYTCIVENQYGSINHTY |
| | | HLDVVERSPHRPILQAGLPANASAVVGGDVEFVCKVYSDAQPHIQWIKHVERN |
| | | GSKYGPDGLPYLQVLKAAGVNTTDKEIEVLYIRNVTFEDAGEYTCLAGNSIGIS |
| | | FHTAWLTVLPAPEKEKEFPTSPDYLEIAIYCIGVFLIACMVLTVILCRMKNTTKK |
| | | PDFSSQPAVHKLTKRIPLRRQVSADSSSSMNSNTPLVRITTRLSSTADAPMLAGV |
| | | SEYELPEDPKWEFPRDKLTLGKPLGEGCFGQVVMAEAVGIDKDRPKEAVTVAV |
| | | KMLKDDATEKDLSDLVSEMEMMKMIGKHKNIINLLGACTQDGPLYVIVEYAS |
| | | KGNLREYLRARRPPGMEYSFDINRVPEEQMTFKDLVSCTYQLARGMEYLASQK |
| | | CIHRDLAARNVLVTENNVMKIADFGLARDINNIDYYKKTTNGRLPVKWMAPE |
| | | ALFDRVYTHQSDVWSFGVLMWEIFTLGGSPYPGIPVEELFKLLKEGHRMDKPA |
| | | NCTNELYMMMRDCWQAVPSQRPTFKQLVEDLDRILTLTTNEEYLDLSGPLEQY |
| | | SPSYPDTRSSCSSGDDSVFSPDPMPYEPCLPKYQHMNGSVKT* |
| 38 | FGF2-N550K | MGLKSTWRYGNGPGTYSKKMVSWDSGCLICLVVVTMAGLSLARPSFNLVVED |
| | | ATLEPEEPPTKYQISQPDVHSALPGEPLELRCQLKDAVMISWTKDGVPLGPDNR |
| | | TVIIGEYLQIKDASPRDSGLYACTAIRTLDSDTLYFIVNVTDALSSGDDEDDNDG |
| | | SEDFVNDSNQMRAPYWTHTDKMEKRLHAVPAANTVKFRCPAMGNPTPTMRW |
| | | LKNGKEFKQEHRIGGYKVRNQHWSLIMESVVPSDKGNYTCIVENQYGSINHTY |
| | | HLDVVERSPHRPILQAGLPANASAVVGGDVEFVCKVYSDAQPHIQWIKHVERN |
| | | GSKYGPDGLPYLQVLKAAGVNTTDKEIEVLYIRNVTFEDAGEYTCLAGNSIGIS |
| | | FHTAWLTVLPAPEKEKEFPTSPDYLEIAIYCIGVFLIACMVLTVILCRMKNTTKK |
| | | PDFSSQPAVHKLTKRIPLRRQVSADSSSSMNSNTPLVRITTRLSSTADAPMLAGV |
| | | SEYELPEDPKWEFPRDKLTLGKPLGEGCFGQVVMAEAVGIDKDRPKEAVTVAV |
| | | KMLKDDATEKDLSDLVSEMEMMKMIGKHKNIIKLLGACTQDGPLYVIVEYAS |
| | | KGNLREYLRARRPPGMEYSFDINRVPEEQMTFKDLVSCTYQLARGMEYLASQK |
| | | CIHRDLAARNVLVTENNVMKIADFGLARDINNIDYYKKTTNGRLPVKWMAPE |
| | | ALFDRVYTHQSDVWSFGVLMWEIFTLGGSPYPGIPVEELFKLLKEGHRMDKPA |
| | | NCTNELYMMMRDCWQAVPSQRPTFKQLVEDLDRILTLTTNEEYLDLSGPLEQY |
| | | SPSYPDTRSSCSSGDDSVFSPDPMPYEPCLPKYQHMNGSVKT* |
| 39 | FGF2-K660E | MGLKSTWRYGNGPGTYSKKMVSWDSGCLICLVVVTMAGLSLARPSFNLVVED |
| | | ATLEPEEPPTKYQISQPDVHSALPGEPLELRCQLKDAVMISWTKDGVPLGPDNR |
| | | TVIIGEYLQIKDASPRDSGLYACTAIRTLDSDTLYFIVNVTDALSSGDDEDDNDG |
| | | SEDFVNDSNQMRAPYWTHTDKMEKRLHAVPAANTVKFRCPAMGNPTPTMRW |
| | | LKNGKEFKQEHRIGGYKVRNQHWSLIMESVVPSDKGNYTCIVENQYGSINHTY |
| | | HLDVVERSPHRPILQAGLPANASAVVGGDVEFVCKVYSDAQPHIQWIKHVERN |
| | | GSKYGPDGLPYLQVLKAAGVNTTDKEIEVLYIRNVTFEDAGEYTCLAGNSIGIS |
| | | FHTAWLTVLPAPEKEKEFPTSPDYLEIAIYCIGVFLIACMVLTVILCRMKNTTKK |
| | | PDFSSQPAVHKLTKRIPLRRQVSADSSSSMNSNTPLVRITTRLSSTADAPMLAGV |
| | | SEYELPEDPKWEFPRDKLTLGKPLGEGCFGQVVMAEAVGIDKDRPKEAVTVAV |
| | | KMLKDDATEKDLSDLVSEMEMMKMIGKHKNIINLLGACTQDGPLYVIVEYAS |
| | | KGNLREYLRARRPPGMEYSFDINRVPEEQMTFKDLVSCTYQLARGMEYLASQK |
| | | CIHRDLAARNVLVTENNVMKIADFGLARDINNIDYYKETTNGRLPVKWMAPE |
| | | ALFDRVYTHQSDVWSFGVLMWEIFTLGGSPYPGIPVEELFKLLKEGHRMDKPA |
| | | NCTNELYMMMRDCWQAVPSQRPTFKQLVEDLDRILTLTTNEEYLDLSGPLEQY |
| | | SPSYPDTRSSCSSGDDSVFSPDPMPYEPCLPKYQHMNGSVKT* |
| 40 | FGF2-N550K- | MGLKSTWRYGNGPGTYSKKMVSWDSGCLICLVVVTMAGLSLARPSFNLVVED |
| | N660E-S780A | ATLEPEEPPTKYQISQPDVHSALPGEPLELRCQLKDAVMISWTKDGVPLGPDNR |
| | | TVIIGEYLQIKDASPRDSGLYACTAIRTLDSDTLYFIVNVTDALSSGDDEDDNDG |
| | | SEDFVNDSNQMRAPYWTHTDKMEKRLHAVPAANTVKFRCPAMGNPTPTMRW |
| | | LKNGKEFKQEHRIGGYKVRNQHWSLIMESVVPSDKGNYTCIVENQYGSINHTY |
| | | HLDVVERSPHRPILQAGLPANASAVVGGDVEFVCKVYSDAQPHIQWIKHVERN |
| | | GSKYGPDGLPYLQVLKAAGVNTTDKEIEVLYIRNVTFEDAGEYTCLAGNSIGIS |
| | | FHTAWLTVLPAPEKEKEFPTSPDYLEIAIYCIGVFLIACMVLTVILCRMKNTTKK |
| | | PDFSSQPAVHKLTKRIPLRRQVSADSSSSMNSNTPLVRITTRLSSTADAPMLAGV |
| | | SEYELPEDPKWEFPRDKLTLGKPLGEGCFGQVVMAEAVGIDKDRPKEAVTVAV |
| | | KMLKDDATEKDLSDLVSEMEMMKMIGKHKNIIKLLGACTQDGPLYVIVEYAS |
| | | KGNLREYLRARRPPGMEYSFDINRVPEEQMTFKDLVSCTYQLARGMEYLASQK |
| | | CIHRDLAARNVLVTENNVMKIADFGLARDINNIDYYKETTNGRLPVKWMAPE |
| | | ALFDRVYTHQSDVWSFGVLMWEIFTLGGSPYPGIPVEELFKLLKEGHRMDKPA |
| | | NCTNELYMMMRDCWQAVPSQRPTFKQLVEDLDRILTLTTNEEYLDLSGPLEQY |
| | | APSYPDTRSSCSSGDDSVFSPDPMPYEPCLPKYQHMNGSVKT* |
| 41 | FGFR3-WT | MSEAGGGAAAAASLPRSRAGGMRAAWGSVWCLCLAAAVGALPAARRRGAE |
| | | RSGGQAAEYLRSETAFLEELVFGSGDTIELSCNTQSSSVSVFWFKDGIGIAPSNR |
| | | THIGQKLLKIINVSYDDSGLYSCKPRHSNEVLGNFTVRVTGVPFWTRPDKMEK |
| | | KLLAVPAANTVRFRCPAGGNPTPTIYWLKNGKEFKGEHRIGGIKLRHQQWSLV |
| | | MESVVPSDRGNYTCVVENKYGNIRHTYQLDVLERSPHRPILQAGLPANQTVVV |
| | | GSNVEFHCKVYSDAQPHIQWLKHVEVNGSKYGPDGTPYVTVLKSWISKNAEA |
| | | DANLNLFNVTEQDEGEYLCRANNFVGIAEKPFWLHIRKPKPAEELMEMDDSGS |
| | | VYAGILSYGTGLVLFILVLVIVIICRMKMPNKKAMNTTTVQKVSKFPLKRQVTV |
| | | SLESNSSMNSNTPLVRITRLSSSDGPMLANVSELELPPDPKWELARSRLTLGKPL |
| | | GEGCFGQVVMAEAIGIDKDKPNKAITVAVKMLKDDATDKDLSDLVSEMEMM |
| | | KMIGKHKNIINLLGACTQDGPLYVLVEYASKGNLREYLRARRPPGMDYSFDTC |
| | | KLPEEQLTFKDLVSCAYQVARGMEYLASQKCIHRDLAARNVLVTEDNVMKIA |
| | | DFGLARDVHNIDYYKKTTNGRLPVKWMAPEALFDRVYTHQSDVWSFGVLLW |
| | | EIFTLGGSPYPGIPVEELFKLLKEGHRMDKPANCTHDLYMIMRECWHAVPSQRP |
| | | TFKQLVEDLDRVLTMTSTDEYLDLSVPFEQYSPAGQDTHSTCSSGDDSVFAHD |
| | | LLPDEPCLPKHVPCNGVIRT* |
| 42 | FGFR3- | MSEAGGGAAAAASLPRSRAGGMRAAWGSVWCLCLAAAVGALPAARRRGAE |
| | N540K | RSGGQAAEYLRSETAFLEELVFGSGDTIELSCNTQSSSVSVFWFKDGIGIAPSNR |
| | | THIGQKLLKIINVSYDDSGLYSCKPRHSNEVLGNFTVRVTGVPFWTRPDKMEK |
| | | KLLAVPAANTVRFRCPAGGNPTPTIYWLKNGKEFKGEHRIGGIKLRHQQWSLV |
| | | MESVVPSDRGNYTCVVENKYGNIRHTYQLDVLERSPHRPILQAGLPANQTVVV |
| | | GSNVEFHCKVYSDAQPHIQWLKHVEVNGSKYGPDGTPYVTVLKSWISKNAEA |
| | | DANLNLFNVTEQDEGEYLCRANNFVGIAEKPFWLHIRKPKPAEELMEMDDSGS |
| | | VYAGILSYGTGLVLFILVLVIVIICRMKMPNKKAMNTTTVQKVSKFPLKRQVTV |
| | | SLESNSSMNSNTPLVRITRLSSSDGPMLANVSELELPPDPKWELARSRLTLGKPL |
| | | GEGCFGQVVMAEAIGIDKDKPNKAITVAVKMLKDDATDKDLSDLVSEMEMM |
| | | KMIGKHKNIIKLLGACTQDGPLYVLVEYASKGNLREYLRARRPPGMDYSFDTC |
| | | KLPEEQLTFKDLVSCAYQVARGMEYLASQKCIHRDLAARNVLVTEDNVMKIA |
| | | DFGLARDVHNIDYYKKTTNGRLPVKWMAPEALFDRVYTHQSDVWSFGVLLW |
| | | EIFTLGGSPYPGIPVEELFKLLKEGHRMDKPANCTHDLYMIMRECWHAVPSQRP |
| | | TFKQLVEDLDRVLTMTSTDEYLDLSVPFEQYSPAGQDTHSTCSSGDDSVFAHD |
| | | LLPDEPCLPKHVPCNGVIRT* |
| 43 | FGFR3- | MSEAGGGAAAAASLPRSRAGGMRAAWGSVWCLCLAAAVGALPAARRRGAE |
| | K650E | RSGGQAAEYLRSETAFLEELVFGSGDTIELSCNTQSSSVSVFWFKDGIGIAPSNR |
| | | THIGQKLLKIINVSYDDSGLYSCKPRHSNEVLGNFTVRVTGVPFWTRPDKMEK |
| | | KLLAVPAANTVRFRCPAGGNPTPTIYWLKNGKEFKGEHRIGGIKLRHQQWSLV |
| | | MESVVPSDRGNYTCVVENKYGNIRHTYQLDVLERSPHRPILQAGLPANQTVVV |
| | | GSNVEFHCKVYSDAQPHIQWLKHVEVNGSKYGPDGTPYVTVLKSWISKNAEA |
| | | DANLNLFNVTEQDEGEYLCRANNFVGIAEKPFWLHIRKPKPAEELMEMDDSGS |
| | | VYAGILSYGTGLVLFILVLVIVIICRMKMPNKKAMNTTTVQKVSKFPLKRQVTV |
| | | SLESNSSMNSNTPLVRITRLSSSDGPMLANVSELELPPDPKWELARSRLTLGKPL |
| | | GEGCFGQVVMAEAIGIDKDKPNKAITVAVKMLKDDATDKDLSDLVSEMEMM |
| | | KMIGKHKNIINLLGACTQDGPLYVLVEYASKGNLREYLRARRPPGMDYSFDTC |
| | | KLPEEQLTFKDLVSCAYQVARGMEYLASQKCIHRDLAARNVLVTEDNVMKIA |
| | | DFGLARDVHNIDYYKETTNGRLPVKWMAPEALFDRVYTHQSDVWSFGVLLW |
| | | EIFTLGGSPYPGIPVEELFKLLKEGHRMDKPANCTHDLYMIMRECWHAVPSQRP |
| | | TFKQLVEDLDRVLTMTSTDEYLDLSVPFEQYSPAGQDTHSTCSSGDDSVFAHD |
| | | LLPDEPCLPKHVPCNGVIRT* |
| 44 | FGFR3- | MSEAGGGAAAAASLPRSRAGGMRAAWGSVWCLCLAAAVGALPAARRRGAE |
| | N540K-K650E | RSGGQAAEYLRSETAFLEELVFGSGDTIELSCNTQSSSVSVFWFKDGIGIAPSNR |
| | | THIGQKLLKIINVSYDDSGLYSCKPRHSNEVLGNFTVRVTGVPFWTRPDKMEK |
| | | KLLAVPAANTVRFRCPAGGNPTPTIYWLKNGKEFKGEHRIGGIKLRHQQWSLV |
| | | MESVVPSDRGNYTCVVENKYGNIRHTYQLDVLERSPHRPILQAGLPANQTVVV |
| | | GSNVEFHCKVYSDAQPHIQWLKHVEVNGSKYGPDGTPYVTVLKSWISKNAEA |
| | | DANLNLFNVTEQDEGEYLCRANNFVGIAEKPFWLHIRKPKPAEELMEMDDSGS |
| | | VYAGILSYGTGLVLFILVLVIVIICRMKMPNKKAMNTTTVQKVSKFPLKRQVTV |
| | | SLESNSSMNSNTPLVRITRLSSSDGPMLANVSELELPPDPKWELARSRLTLGKPL |
| | | GEGCFGQVVMAEAIGIDKDKPNKAITVAVKMLKDDATDKDLSDLVSEMEMM |
| | | KMIGKHKNIIKLLGACTQDGPLYVLVEYASKGNLREYLRARRPPGMDYSFDTC |
| | | KLPEEQLTFKDLVSCAYQVARGMEYLASQKCIHRDLAARNVLVTEDNVMKIA |
| | | DFGLARDVHNIDYYKETTNGRLPVKWMAPEALFDRVYTHQSDVWSFGVLLW |
| | | EIFTLGGSPYPGIPVEELFKLLKEGHRMDKPANCTHDLYMIMRECWHAVPSQRP |
| | | TFKQLVEDLDRVLTMTSTDEYLDLSVPFEQYSPAGQDTHSTCSSGDDSVFAHD |
| | | LLPDEPCLPKHVPCNGVIRT* |
| 45 | FGFR3- | MGSSKSKPKDPSQRRMKMPNKKAMNTTTVQKVSKFPLKRQVTVSLESNSSMN |
| | myrist-K650E | SNTPLVRITRLSSSDGPMLANVSELELPPDPKWELARSRLTLGKPLGEGCFGQV |
| | | VMAEAIGIDKDKPNKAITVAVKMLKDDATDKDLSDLVSEMEMMKMIGKHKNI |
| | | INLLGACTQDGPLYVLVEYASKGNLREYLRARRPPGMDYSFDTCKLPEEQLTF |
| | | KDLVSCAYQVARGMEYLASQKCIHRDLAARNVLVTEDNVMKIADFGLARDV |
| | | HNIDYYKETTNGRLPVKWMAPEALFDRVYTHQSDVWSFGVLLWEIFTLGGSP |
| | | YPGIPVEELFKLLKEGHRMDKPANCTHDLYMIMRECWHAVPSQRPTFKQLVED |
| | | LDRVLTMTSTDEYLDLSVPFEQYSPAGQDTHSTCSSGDDSVFAHDLLPDEPCLP |
| | | KHVPCNGVIRT* |
| 46 | FGFR4-WT | MLPLRLVLAGLLVAAGSAASHRGEMEPELFESPLLESEEEHLLLDPGNALKLYC |
| | | DVNQSGASVVWYKESRPLLPGPRVRLQQSVLEIAEVAYEDSGLYVCRARGTGE |
| | | VLRNFTISVVDSLASGDDDEDSDGDGPHGDRSEEPVYVHRAPYWTHPHRMDK |
| | | KLYAVPAGNTVKFRCPASGSPSPSIRWFKNGREFRGEHRIGGIRLRHQHWSLVM |
| | | ESVVPSDRGNYTCLVENRFGSIRYSYLLDVLERSPHRPILQAGLPANTTALVGSD |
| | | VEFFCKVYSDAQPHIQWLKHIEVNGSSYGPDGVPYVQVLKTADINSSEVEVLYL |
| | | RNVTMEDAGEYTCLAGNSIGLSYQSAWLTVLPEELVHEAEAPEAKYTDIIIYTS |
| | | GSLAVAMALIIVVLCRMQTQSSKQPLEPMAVHKLSKFPLIRQFSLDSSSSGKSST |
| | | SLMRVTRLSSSCAPMLAGVVELDLPLDSKWEFPREKLVLGKPLGEGCFGQVVR |
| | | AEAYGIDRQWPDRAVTVAVKMLKDNATDKDLADLISEMEMMKLMDKHKNII |
| | | NLLGVCTQDGPLYVIVEFAAKGNLREYLRARRPPMPDYTFDITELHEEQLCFKD |
| | | LVSCVYQVARGMEYLESRRCIHRDLAARNVLVTAENVMKIADFGLARDVHDI |
| | | DYYKKTSNGRLPVKWMAPEALFDRVYTHQSDVWSFGILMWEIFTLGGSPYPGI |
| | | PVEELFKLLKEGHRMDCPSNCTHELYMLMRECWHAVPSQRPTFKQLVEGLDKI |
| | | LAAISEEYLDLSMPFEQYSPSCEDTTSTCSSDDSVFTHDPLPLAPCLFACPSGRT* |
| 47 | FGFR4- | MLPLRLVLAGLLVAAGSAASHRGEMEPELFESPLLESEEEHLLLDPGNALKLYC |
| | Y367C | DVNQSGASVVWYKESRPLLPGPRVRLQQSVLEIAEVAYEDSGLYVCRARGTGE |
| | | VLRNFTISVVDSLASGDDDEDSDGDGPHGDRSEEPVYVHRAPYWTHPHRMDK |
| | | KLYAVPAGNTVKFRCPASGSPSPSIRWFKNGREFRGEHRIGGIRLRHQHWSLVM |
| | | ESVVPSDRGNYTCLVENRFGSIRYSYLLDVLERSPHRPILQAGLPANTTALVGSD |
| | | VEFFCKVYSDAQPHIQWLKHIEVNGSSYGPDGVPYVQVLKTADINSSEVEVLYL |
| | | RNVTMEDAGEYTCLAGNSIGLSYQSAWLTVLPEELVHEAEAPEAKCTDIIIYTS |
| | | GSLAVAMALIIVVLCRMQTQSSKQPLEPMAVHKLSKFPLIRQFSLDSSSSGKSST |
| | | SLMRVTRLSSSCAPMLAGVVELDLPLDSKWEFPREKLVLGKPLGEGCFGQVVR |
| | | AEAYGIDRQWPDRAVTVAVKMLKDNATDKDLADLISEMEMMKLMDKHKNII |
| | | NLLGVCTQDGPLYVIVEFAAKGNLREYLRARRPPMPDYTFDITELHEEQLCFKD |
| | | LVSCVYQVARGMEYLESRRCIHRDLAARNVLVTAENVMKIADFGLARDVHDI |
| | | DYYKKTSNGRLPVKWMAPEALFDRVYTHQSDVWSFGILMWEIFTLGGSPYPGI |
| | | PVEELFKLLKEGHRMDCPSNCTHELYMLMRECWHAVPSQRPTFKQLVEGLDKI |
| | | LAAISEEYLDLSMPFEQYSPSCEDTTSTCSSDDSVFTHDPLPLAPCLFACPSGRT* |
| 48 | FGFR4- | MLPLRLVLAGLLVAAGSAASHRGEMEPELFESPLLESEEEHLLLDPGNALKLYC |
| | K645E | DVNQSGASVVWYKESRPLLPGPRVRLQQSVLEIAEVAYEDSGLYVCRARGTGE |
| | | VLRNFTISVVDSLASGDDDEDSDGDGPHGDRSEEPVYVHRAPYWTHPHRMDK |
| | | KLYAVPAGNTVKFRCPASGSPSPSIRWFKNGREFRGEHRIGGIRLRHQHWSLVM |
| | | ESVVPSDRGNYTCLVENRFGSIRYSYLLDVLERSPHRPILQAGLPANTTALVGSD |
| | | VEFFCKVYSDAQPHIQWLKHIEVNGSSYGPDGVPYVQVLKTADINSSEVEVLYL |
| | | RNVTMEDAGEYTCLAGNSIGLSYQSAWLTVLPEELVHEAEAPEAKYTDIIIYTS |
| | | GSLAVAMALIIVVLCRMQTQSSKQPLEPMAVHKLSKFPLIRQFSLDSSSSGKSST |
| | | SLMRVTRLSSSCAPMLAGVVELDLPLDSKWEFPREKLVLGKPLGEGCFGQVVR |
| | | AEAYGIDRQWPDRAVTVAVKMLKDNATDKDLADLISEMEMMKLMDKHKNII |
| | | NLLGVCTQDGPLYVIVEFAAKGNLREYLRARRPPMPDYTFDITELHEEQLCFKD |
| | | LVSCVYQVARGMEYLESRRCIHRDLAARNVLVTAENVMKIADFGLARDVHDI |
| | | DYYKETSNGRLPVKWMAPEALFDRVYTHQSDVWSFGILMWEIFTLGGSPYPGI |
| | | PVEELFKLLKEGHRMDCPSNCTHELYMLMRECWHAVPSQRPTFKQLVEGLDKI |
| | | LAAISEEYLDLSMPFEQYSPSCEDTTSTCSSDDSVFTHDPLPLAPCLFACPSGRT* |
| 49 | FGFR4- | MLPLRLVLAGLLVAAGSAASHRGEMEPELFESPLLESEEEHLLLDPGNALKLYC |
| | Y367C-K645E | DVNQSGASVVWYKESRPLLPGPRVRLQQSVLEIAEVAYEDSGLYVCRARGTGE |
| | | VLRNFTISVVDSLASGDDDEDSDGDGPHGDRSEEPVYVHRAPYWTHPHRMDK |
| | | KLYAVPAGNTVKFRCPASGSPSPSIRWFKNGREFRGEHRIGGIRLRHQHWSLVM |
| | | ESVVPSDRGNYTCLVENRFGSIRYSYLLDVLERSPHRPILQAGLPANTTALVGSD |
| | | VEFFCKVYSDAQPHIQWLKHIEVNGSSYGPDGVPYVQVLKTADINSSEVEVLYL |
| | | RNVTMEDAGEYTCLAGNSIGLSYQSAWLTVLPEELVHEAEAPEAKCTDIIIYTS |
| | | GSLAVAMALIIVVLCRMQTQSSKQPLEPMAVHKLSKFPLIRQFSLDSSSSGKSST |
| | | SLMRVTRLSSSCAPMLAGVVELDLPLDSKWEFPREKLVLGKPLGEGCFGQVVR |
| | | AEAYGIDRQWPDRAVTVAVKMLKDNATDKDLADLISEMEMMKLMDKHKNII |
| | | NLLGVCTQDGPLYVIVEFAAKGNLREYLRARRPPMPDYTFDITELHEEQLCFKD |
| | | LVSCVYQVARGMEYLESRRCIHRDLAARNVLVTAENVMKIADFGLARDVHDI |
| | | DYYKETSNGRLPVKWMAPEALFDRVYTHQSDVWSFGILMWEIFTLGGSPYPGI |
| | | PVEELFKLLKEGHRMDCPSNCTHELYMLMRECWHAVPSQRPTFKQLVEGLDKI |
| | | LAAISEEYLDLSMPFEQYSPSCEDTTSTCSSDDSVFTHDPLPLAPCLFACPSGRT* |
| 50 | ggIGFR1-WT | MKSGAGGGTLAVFCGLLLAFAALCLCPTNGEICGPNVDIRNDIHELKRLENCTV |
| | | VEGFLQILLISKAEDYRNFRFPKLTVITDYLLLFRVAGLESLSDLFPNLTVIRGRN |
| | | LFYNYALVIFEMTNLKEIGLHNLRNITRGAIRIEKNSDLCYLSTVDWSLILDAVS |
| | | NNYIVGNKPPKECGDLCPGTMEEKPLCEKTSINNEYNYRCWTTNHCQKMCPSS |
| | | CGKRACTDQNECCHPECLGSCTAPDNNTACVACRNYYYEGVCMPTCPPNTYK |
| | | FEGWRCVTKEFCSKVPATETSDYERFVIHNDECMAECPSGFIRNGSQSMFCSPC |
| | | EGPCPKICEDGKTKTIDSVTSAQMLQGCTILKGNLLINIRRGNNIASELENFMGLI |
| | | ETVTGYVKIRHSHALVSLSFLKNLRYILGEEQVDGNYSFYVLDNHNLQQLWD |
| | | WNHHNLTIKEGKMYFAFNPKLCVSEIYRMEEVSGTKGRQSKGDINPRNNGERA |
| | | SCESHILRFVSNTTLKNRIKLTWERYRPPDYRDLISFTVYYKEAPFKNVTEYDGQ |
| | | DACGSNSWNMVDVDLPPNKENDPGILLQGLKPWTQYAIYVKAVTLTMMENH |
| | | HIHGAKSEIVYIRTNAAVPSIPLDVISASNSSSQLIVKWNPPSLPNGNLSYYIVRW |
| | | QQQPQDSYLYRHNYCSKDKVPIRRYADGTIDTEEATEPTKPEGCGGEKGPCCA |
| | | CPKTEAEKQAEKEEAEYRKVFENFLHNSIFVPRPDRKRRDVFRIANATLATRNR |
| | | NITGADHFTNASDAEESEVEYPFFETKVDGKERTVISHLQPFTLYRIDIHSCNHE |
| | | ADTLGCSASNFVFARTMPSEGADNIPGTVAWEAKEENTVYLKWLEPTNPNGLI |
| | | LMYEIKYGQHGEEKRECVSRQEYKKLGGAKLTHLNPGNYSARVQATSLAGNG |
| | | SWTEPVSFYVQPKSANYDNFLHLIIVLPIAFLLIIGGLLIMLYVFNKKRNSDRLGN |
| | | GVLYASVNPEYFSASDVYVPDEWEVPREKITMCRELGQGSFGMVYEGIAKGV |
| | | VKDEPETRVAIKTVNESASMRERIEFLNEASVMKEFNCHHVVRLLGVVSQGQP |
| | | TLVIMELMTRGDLKSYLRSLRPDTESNPGQAPPTLKKMIQMAGEIADGMAYLN |
| | | ANKFVHRDLAARNCMVAEDFTVKIGDFGMTRDIYETDYYRKGGKGLLPVRW |
| | | MSPESLKDGVFTTHSDVWSFGVVLWEIATLAEQPYQGMTNEQVLRFVMEGGL |
| | | LEKPDNCPDMLFELMRMCWQYNPKMRPSFLEIISSIKDELDPAFKEVSFFYSEE |
| | | NKPPDTEELDLETENMESIPLDPSSTLQPTDKHSGHKAENGPGVVVLRASFEER |
| | | QPYAHMNGGRKNERALPLPQSSAC* |
| 51 | ggIGFR1- | MKSGAGGGTLAVFCGLLLAFAALCLCPTNGEICGPNVDIRNDIHELKRLENCTV |
| | H1353H | VEGFLQILLISKAEDYRNFRFPKLTVITDYLLLFRVAGLESLSDLFPNLTVIRGRN |
| | | LFYNYALVIFEMTNLKEIGLHNLRNITRGAIRIEKNSDLCYLSTVDWSLILDAVS |
| | | NNYIVGNKPPKECGDLCPGTMEEKPLCEKTSINNEYNYRCWTTNHCQKMCPSS |
| | | CGKRACTDQNECCHPECLGSCTAPDNNTACVACRNYYYEGVCMPTCPPNTYK |
| | | FEGWRCVTKEFCSKVPATETSDYERFVIHNDECMAECPSGFIRNGSQSMFCSPC |
| | | EGPCPKICEDGKTKTIDSVTSAQMLQGCTILKGNLLINIRRGNNIASELENFMGLI |
| | | ETVTGYVKIRHSHALVSLSFLKNLRYILGEEQVDGNYSFYVLDNHNLQQLWD |
| | | WNHHNLTIKEGKMYFAFNPKLCVSEIYRMEEVSGTKGRQSKGDINPRNNGERA |
| | | SCESHILRFVSNTTLKNRIKLTWERYRPPDYRDLISFTVYYKEAPFKNVTEYDGQ |
| | | DACGSNSWNMVDVDLPPNKENDPGILLQGLKPWTQYAIYVKAVTLTMMENH |
| | | HIHGAKSEIVYIRTNAAVPSIPLDVISASNSSSQLIVKWNPPSLPNGNLSYYIVRW |
| | | QQQPQDSYLYRHNYCSKDKVPIRRYADGTIDTEEATEPTKPEGCGGEKGPCCA |
| | | CPKTEAEKQAEKEEAEYRKVFENFLHNSIFVPRPDRKRRDVFRIANATLATRNR |
| | | NITGADHFTNASDAEESEVEYPFFETKVDGKERTVISHLQPFTLYRIDIHSCNHE |
| | | ADTLGCSASNFVFARTMPSEGADNIPGTVAWEAKEENTVYLKWLEPTNPNGLI |
| | | LMYEIKYGQHGEEKRECVSRQEYKKLGGAKLTHLNPGNYSARVQATSLAGNG |
| | | SWTEPVSFYVQPKSANYDNFLHLIIVLPIAFLLIIGGLLIMLYVFNKKRNSDRLGN |
| | | GVLYASVNPEYFSASDVYVPDEWEVPREKITMCRELGQGSFGMVYEGIAKGV |
| | | VKDEPETRVAIKTVNESASMRERIEFLNEASVMKEFNCHHVVRLLGVVSQGQP |
| | | TLVIMELMTRGDLKSYLRSLRPDTESNPGQAPPTLKKMIQMAGEIADGMAYLN |
| | | ANKFVHRDLAARNCMVAEDFTVKIGDFGMTRDIYETDYYRKGGKGLLPVRW |
| | | MSPESLKDGVFTTHSDVWSFGVVLWEIATLAEQPYQGMTNEQVLRFVMEGGL |
| | | LEKPDNCPDMLFELMRMCWQYNPKMRPSFLEIISSIKDELDPAFKEVSFFYSEE |
| | | NKPPDTEELDLETENMESIPLDPSSTLQPTDKHSGHKAENGPGVVVLRASFEER |
| | | QPYAHMNGGRKNEHALPLPQSSAC* |
| 52 | gPDGFRA- | MGTPPRTFLILGCFLTGPLLTLCQLPLPTIVPNRNEMVVQLNSNFTLKCSGDSEV |
| | WT | SWQYPVTEGSHRIDIRHEENNSGLFVTVLEVGNASAAHTGMYVCYYNHTQVE |
| | | DGEVEGKDIYIYVPDPDMPFVPSLPEDQFILVEEGDPTVIPCRTSDPSAEVTLVNS |
| | | LDKPVYAFYDSKQGFVGNFLAGPYTCKTMVKGVEFKSDEFLIYILRATSQLPVE |
| | | IEALKTVYKTGETIVVTCVVFDNEVVNLQWNYPGKVKEKGLIKLDDIKVPSQK |
| | | LVYTLTIPDASVKDTGDYECTARHATKEVKENKKVVITVHDKGFIHLEPQFSPL |
| | | EAVNLHEVKNFVVDVQAYPAPKMYWLKDNVTLIENLTEIVTSSNRVQETRFQS |
| | | VLKLIRAKEEDSGYYTLVAENEDEIKRYTFSLLIQVPALILDLMDDHQGSAGRQ |
| | | TVRCLAEGTPLPDVEWLVCKDIKKCSNDTSWTLLTNNISDIHMEAHLDERNMV |
| | | ESQVTFQKVEETLAVRCVARNDLGAVTRELKLVAPTLRSELTVAAAVLVLLVI |
| | | VIISLIVLVIIWKQKPRYEIRWRVIESISPDGHEYIYVDPMQLPYDSRWEFPRDGL |
| | | VLGRILGSGAFGKVVEGTAYGLSRSQPVMKVAVKMLKPTARSSEKQALMSEL |
| | | KIMTHLGPHLNIVNLLGACTKSGPIYIITEYCFYGDLVNYLHKNRDNFLSRHPEK |
| | | PKKDLDIFGMNPADESTRSYVILSFENTGEYMDMKQADTTQYVPMLERKEGSK |
| | | YSDIQRSVYDRPASYKKKSLSESEVKNLLSDDGSEGLSLLDLLSFTYQVARGME |
| | | FLASKNCVHRDLAARNVLLAQGKIVKICDFGLARDIMHDSNYVSKGSTFLPVK |
| | | WMAPESIFDNLYTTLSDVWSYGILLWEIFSLGGTPYPGMMVDSTFYNKIKSGYR |
| | | MAKPDHATNEVYEIMVKCWNSEPEKRPSFYHLSEIVESLLPGEYKKSYEKIHLD |
| | | FLKSDHPAVTRMRGDCDNAYIGVTYKNEDKIKDRESGFDEQRLSADSGYIIPLP |
| | | DIDPVSEDELGKRNRHSSQTSEESAIETGSSSSTFIKREDETIEDIDMMDDIGIDSS |
| | | DLVEDSFL* |
| 53 | ggPDGFRA- | MGTPPRTFLILGCFLTGPLLTLCQLPLPTIVPNRNEMVVQLNSNFTLKCSGDSEV |
| | D842V | SWQYPVTEGSHRIDIRHEENNSGLFVTVLEVGNASAAHTGMYVCYYNHTQVE |
| | | DGEVEGKDIYIYVPDPDMPFVPSLPEDQFILVEEGDPTVIPCRTSDPSAEVTLVNS |
| | | LDKPVYAFYDSKQGFVGNFLAGPYTCKTMVKGVEFKSDEFLIYILRATSQLPVE |
| | | IEALKTVYKTGETIVVTCVVFDNEVVNLQWNYPGKVKEKGLIKLDDIKVPSQK |
| | | LVYTLTIPDASVKDTGDYECTARHATKEVKENKKVVITVHDKGFIHLEPQFSPL |
| | | EAVNLHEVKNFVVDVQAYPAPKMYWLKDNVTLIENLTEIVTSSNRVQETRFQS |
| | | VLKLIRAKEEDSGYYTLVAENEDEIKRYTFSLLIQVPALILDLMDDHQGSAGRQ |
| | | TVRCLAEGTPLPDVEWLVCKDIKKCSNDTSWTLLTNNISDIHMEAHLDERNMV |
| | | ESQVTFQKVEETLAVRCVARNDLGAVTRELKLVAPTLRSELTVAAAVLVLLVI |
| | | VIISLIVLVIIWKQKPRYEIRWRVIESISPDGHEYIYVDPMQLPYDSRWEFPRDGL |
| | | VLGRILGSGAFGKVVEGTAYGLSRSQPVMKVAVKMLKPTARSSEKQALMSEL |
| | | KIMTHLGPHLNIVNLLGACTKSGPIYIITEYCFYGDLVNYLHKNRDNFLSRHPEK |
| | | PKKDLDIFGMNPADESTRSYVILSFENTGEYMDMKQADTTQYVPMLERKEGSK |
| | | YSDIQRSVYDRPASYKKKSLSESEVKNLLSDDGSEGLSLLDLLSFTYQVARGME |
| | | FLASKNCVHRDLAARNVLLAQGKIVKICDFGLARVIMHDSNYVSKGSTFLPVK |
| | | WMAPESIFDNLYTTLSDVWSYGILLWEIFSLGGTPYPGMMVDSTFYNKIKSGYR |
| | | MAKPDHATNEVYEIMVKCWNSEPEKRPSFYHLSEIVESLLPGEYKKSYEKIHLD |
| | | FLKSDHPAVTRMRGDCDNAYIGVTYKNEDKIKDRESGFDEQRLSADSGYIIPLP |
| | | DIDPVSEDELGKRNRHSSQTSEESAIETGSSSSTFIKREDETIEDIDMMDDIGIDSS |
| | | DLVEDSFL* |
| 54 | ggPDGFRBWT | MLCPSLKASLQLLILTGLLEVTSGGSGLHIEPEDAELVLRLHSTFSLVCYGDGTL |
| | | VWERDGQPLTAVLEHRDGVFISNLTLRNVTGRHTGEYACFYSPDQAPERAERK |
| | | ALYIYVPDPSLVFLPAITSEEFFIFITGYTEATIPCRVTNPELQVTLYEKKVENPIP |
| | | ATYDPQQGFKGFFEDKTYYCQAIVDDQEVDSDTFYVYRIQVSSVNVSISAVQT |
| | | VVRQGENVTLMCTVSGNELVNFNWDYPRKQAGKAVEPVTDFLPGSTHDIRSIL |
| | | IIQNAELEDSGTYVCNVSEGYHEKTDRKDITVQVIERGFVRFHTHLASTVYAEV |
| | | HKSHIIQVDVEAYPQPNIVWLKNNKTLTMESSSEFTITNRNLSETRYQTSLVLVR |
| | | VKQEEGGYYTIRASNEDDAQELSFHLQINVPAKVVDLKENSSASSGEQTVTCSA |
| | | EGMPQPEISWSTCSNIKWCGSQGQPTQLLGNNSAEIGLHTNATYHAELQVYRV |
| | | NSTLQLHRVDEPLLLRCTVQNFLGSNSQDITLVPNALPFKVVIISVILALLVLTVI |
| | | SLIILIILWQKKPRYEIRWKVIESVSSDGHEYIYVDPMQLPYDSSWEVPRDKLVL |
| | | GRTLGSGAFGRVVEATAHGLSHSQSTMKVAVKMLKSTARSSEKQALMSELKI |
| | | MSHLGPHLNIVNLLGACTKGGPIYIITEYCRYGDLVDYLHRNKHTFLQSYGEKA |
| | | RREAELYGNTIKEDHVQSHLSLSVESDGGYMDMSKDESLDYVPMSDMKGEVK |
| | | YADIESSNYGTPYELDSYSPSAPERTDRVTLINESPLLSYMDLVGFSFQVANGM |
| | | EFLASKNCVHRDLAARNVLICEGKLVKICDFGLARDIMRDSNYISKGSTFLPLK |
| | | WMAPESIFNNLYTTLSDVWSFGILLWEIFTLGGTPYPELPMNEQFYNAIKRGYR |
| | | MSKPTHASDEIYDIMQKCWEEKFEIRPSFSQLVVLMGNLLVDCYRKRYQQVDE |
| | | EFMKSDHPAVVRTRPTIPGLNNARLPPSSPTLYTAVHQNGGENDYIIPLPDPKPD |
| | | AICDLPQEASVSRASSMLNEANTSSTISCDSPLGPRQDEEPECDLQLGCQELAPG |
| | | HHEVEESFL* |
| 55 | ggPDGFRB- | MLCPSLKASLQLLILTGLLEVTSGGSGLHIEPEDAELVLRLHSTFSLVCYGDGTL |
| | V536A | VWERDGQPLTAVLEHRDGVFISNLTLRNVTGRHTGEYACFYSPDQAPERAERK |
| | | ALYIYVPDPSLVFLPAITSEEFFIFITGYTEATIPCRVTNPELQVTLYEKKVENPIP |
| | | ATYDPQQGFKGFFEDKTYYCQAIVDDQEVDSDTFYVYRIQVSSVNVSISAVQT |
| | | VVRQGENVTLMCTVSGNELVNFNWDYPRKQAGKAVEPVTDFLPGSTHDIRSIL |
| | | IIQNAELEDSGTYVCNVSEGYHEKTDRKDITVQVIERGFVRFHTHLASTVYAEV |
| | | HKSHIIQVDVEAYPQPNIVWLKNNKTLTMESSSEFTITNRNLSETRYQTSLVLVR |
| | | VKQEEGGYYTIRASNEDDAQELSFHLQINVPAKVVDLKENSSASSGEQTVTCSA |
| | | EGMPQPEISWSTCSNIKWCGSQGQPTQLLGNNSAEIGLHTNATYHAELQVYRV |
| | | NSTLQLHRVDEPLLLRCTVQNFLGSNSQDITLVPNALPFKVVIISVILALLVLTVI |
| | | SLIILIILWQKKPRYEIRWKAIESVSSDGHEYIYVDPMQLPYDSSWEVPRDKLVL |
| | | GRTLGSGAFGRVVEATAHGLSHSQSTMKVAVKMLKSTARSSEKQALMSELKI |
| | | MSHLGPHLNIVNLLGACTKGGPIYIITEYCRYGDLVDYLHRNKHTFLQSYGEKA |
| | | RREAELYGNTIKEDHVQSHLSLSVESDGGYMDMSKDESLDYVPMSDMKGEVK |
| | | YADIESSNYGTPYELDSYSPSAPERTDRVTLINESPLLSYMDLVGFSFQVANGM |
| | | EFLASKNCVHRDLAARNVLICEGKLVKICDFGLARDIMRDSNYISKGSTFLPLK |
| | | WMAPESIFNNLYTTLSDVWSFGILLWEIFTLGGTPYPELPMNEQFYNAIKRGYR |
| | | MSKPTHASDEIYDIMQKCWEEKFEIRPSFSQLVVLMGNLLVDCYRKRYQQVDE |
| | | EFMKSDHPAVVRTRPTIPGLNNARLPPSSPTLYTAVHQNGGENDYIIPLPDPKPD |
| | | AICDLPQEASVSRASSMLNEANTSSTISCDSPLGPRQDEEPECDLQLGCQELAPG |
| | | HHEVEESFL* |
| 56 | ggPDGFRB- | MLCPSLKASLQLLILTGLLEVTSGGSGLHIEPEDAELVLRLHSTFSLVCYGDGTL |
| | D850V | VWERDGQPLTAVLEHRDGVFISNLTLRNVTGRHTGEYACFYSPDQAPERAERK |
| | | ALYIYVPDPSLVFLPAITSEEFFIFITGYTEATIPCRVTNPELQVTLYEKKVENPIP |
| | | ATYDPQQGFKGFFEDKTYYCQAIVDDQEVDSDTFYVYRIQVSSVNVSISAVQT |
| | | VVRQGENVTLMCTVSGNELVNFNWDYPRKQAGKAVEPVTDFLPGSTHDIRSIL |
| | | IIQNAELEDSGTYVCNVSEGYHEKTDRKDITVQVIERGFVRFHTHLASTVYAEV |
| | | HKSHIIQVDVEAYPQPNIVWLKNNKTLTMESSSEFTITNRNLSETRYQTSLVLVR |
| | | VKQEEGGYYTIRASNEDDAQELSFHLQINVPAKVVDLKENSSASSGEQTVTCSA |
| | | EGMPQPEISWSTCSNIKWCGSQGQPTQLLGNNSAEIGLHTNATYHAELQVYRV |
| | | NSTLQLHRVDEPLLLRCTVQNFLGSNSQDITLVPNALPFKVVIISVILALLVLTVI |
| | | SLIILIILWQKKPRYEIRWKVIESVSSDGHEYIYVDPMQLPYDSSWEVPRDKLVL |
| | | GRTLGSGAFGRVVEATAHGLSHSQSTMKVAVKMLKSTARSSEKQALMSELKI |
| | | MSHLGPHLNIVNLLGACTKGGPIYIITEYCRYGDLVDYLHRNKHTFLQSYGEKA |
| | | RREAELYGNTIKEDHVQSHLSLSVESDGGYMDMSKDESLDYVPMSDMKGEVK |
| | | YADIESSNYGTPYELDSYSPSAPERTDRVTLINESPLLSYMDLVGFSFQVANGM |
| | | EFLASKNCVHRDLAARNVLICEGKLVKICDFGLARDIMRNSNYISKGSTFLPLK |
| | | WMAPESIFNNLYTTLSDVWSFGILLWEIFTLGGTPYPELPMNEQFYNAIKRGYR |
| | | MSKPTHASDEIYDIMQKCWEEKFEIRPSFSQLVVLMGNLLVDCYRKRYQQVDE |
| | | EFMKSDHPAVVRTRPTIPGLNNARLPPSSPTLYTAVHQNGGENDYIIPLPDPKPD |
| | | AICDLPQEASVSRASSMLNEANTSSTISCDSPLGPRQDEEPECDLQLGCQELAPG |
| | | HHEVEESFL* |
| 57 | F-9C- | MADYKDDDDKKGGIIVAILLLIVMLAIEILLLITLIIAVTSGGSGLHIEPEDAELVL |
| | PRggPDGFRB | RLHSTFSLVCYGDGTLVWERDGQPLTAVLEHRDGVFISNLTLRNVTGRHTGEY |
| | | ACFYSPDQAPERAERKALYIYVPDPSLVFLPAITSEEFFIFITGYTEATIPCRVTNP |
| | | ELQVTLYEKKVENPIPATYDPQQGFKGFFEDKTYYCQAIVDDQEVDSDTFYVY |
| | | RIQVSSVNVSISAVQTVVRQGENVTLMCTVSGNELVNFNWDYPRKQAGKAVE |
| | | PVTDFLPGSTHDIRSILIIQNAELEDSGTYVCNVSEGYHEKTDRKDITVQVIERGF |
| | | VRFHTHLASTVYAEVHKSHIIQVDVEAYPQPNIVWLKNNKTLTMESSSEFTITN |
| | | RNLSETRYQTSLVLVRVKQEEGGYYTIRASNEDDAQELSFHLQINVPAKVVDL |
| | | KENSSASSGEQTVTCSAEGMPQPEISWSTCSNIKWCGSQGQPTQLLGNNSAEIG |
| | | LHTNATYHAELQVYRVNSTLQLHRVDEPLLLRCTVQNFLGSNSQDITLVPNALP |
| | | FKVVIISVILALLVLTVISLIILIILWQKKPRYEIRWKVIESVSSDGHEYIYVDPMQ |
| | | LPYDSSWEVPRDKLVLGRTLGSGAFGRVVEATAHGLSHSQSTMKVAVKMLKS |
| | | TARSSEKQALMSELKIMSHLGPHLNIVNLLGACTKGGPIYIITEYCRYGDLVDYL |
| | | HRNKHTFLQSYGEKARREAELYGNTIKEDHVQSHLSLSVESDGGYMDMSKDE |
| | | SLDYVPMSDMKGEVKYADIESSNYGTPYELDSYSPSAPERTDRVTLINESPLLS |
| | | YMDLVGFSFQVANGMEFLASKNCVHRDLAARNVLICEGKLVKICDFGLARDI |
| | | MRDSNYISKGSTFLPLKWMAPESIFNNLYTTLSDVWSFGILLWEIFTLGGTPYPE |
| | | LPMNEQFYNAIKRGYRMSKPTHASDEIYDIMQKCWEEKFEIRPSFSQLVVLMG |
| | | NLLVDCYRKRYQQVDEEFMKSDHPAVVRTRPTIPGLNNARLPPSSPTLYTAVH |
| | | QNGGENDYIIPLPDPKPDAICDLPQEASVSRASSMLNEANTSSTISCDSPLGPRQD |
| | | EEPECDLQLGCQELAPGHHEVEESFL* |
| 58 | 9C- | MAKGGIIVAILLLIVMLAIEILLLITLIIAVTSGGSGLHIEPEDAELVLRLHSTFSLV |
| | PRggPDGFRB | CYGDGTLVWERDGQPLTAVLEHRDGVFISNLTLRNVTGRHTGEYACFYSPDQA |
| | | PERAERKALYIYVPDPSLVFLPAITSEEFFIFITGYTEATIPCRVTNPELQVTLYEK |
| | | KVENPIPATYDPQQGFKGFFEDKTYYCQAIVDDQEVDSDTFYVYRIQVSSVNVS |
| | | ISAVQTVVRQGENVTLMCTVSGNELVNFNWDYPRKQAGKAVEPVTDFLPGST |
| | | HDIRSILIIQNAELEDSGTYVCNVSEGYHEKTDRKDITVQVIERGFVRFHTHLAS |
| | | TVYAEVHKSHIIQVDVEAYPQPNIVWLKNNKTLTMESSSEFTITNRNLSETRYQ |
| | | TSLVLVRVKQEEGGYYTIRASNEDDAQELSFHLQINVPAKVVDLKENSSASSGE |
| | | QTVTCSAEGMPQPEISWSTCSNIKWCGSQGQPTQLLGNNSAEIGLHTNATYHAE |
| | | LQVYRVNSTLQLHRVDEPLLLRCTVQNFLGSNSQDITLVPNALPFKVVIISVILA |
| | | LLVLTVISLIILIILWQKKPRYEIRWKVIESVSSDGHEYIYVDPMQLPYDSSWEVP |
| | | RDKLVLGRTLGSGAFGRVVEATAHGLSHSQSTMKVAVKMLKSTARSSEKQAL |
| | | MSELKIMSHLGPHLNIVNLLGACTKGGPIYIITEYCRYGDLVDYLHRNKHTFLQ |
| | | SYGEKARREAELYGNTIKEDHVQSHLSLSVESDGGYMDMSKDESLDYVPMSD |
| | | MKGEVKYADIESSNYGTPYELDSYSPSAPERTDRVTLINESPLLSYMDLVGFSF |
| | | QVANGMEFLASKNCVHRDLAARNVLICEGKLVKICDFGLARDIMRDSNYISKG |
| | | STFLPLKWMAPESIFNNLYTTLSDVWSFGILLWEIFTLGGTPYPELPMNEQFYNA |
| | | IKRGYRMSKPTHASDEIYDIMQKCWEEKFEIRPSFSQLVVLMGNLLVDCYRKR |
| | | YQQVDEEFMKSDHPAVVRTRPTIPGLNNARLPPSSPTLYTAVHQNGGENDYIIP |
| | | LPDPKPDAICDLPQEASVSRASSMLNEANTSSTISCDSPLGPRQDEEPECDLQLG |
| | | CQELAPGHHEVEESFL* |
| 59 | FGFBP | MWIKNVGLLCVLILVSQMLLASCERQKERRRGKQGIEHGGKKQAESNPEREKG |
| | | RKPKGGKASPKGKFKSKENADCSWAVTDMSAATVHIECRNGDSAFWCEFSGD |
| | | PSACPHYAANQKSYWKQVSRSLKKQKQICQDPRSILKPKICRKGPRGAHLKLT |
| | | RSSLLAAVDPAKGHPAHHAAEDAQGPAASETGKQPEHSPPDCVEDVDYIDQRK |
| | | VAEEYCPESLLSLCNFFITMVQDKKC |
| 60 | RASV12 | MTEYKLVVVGAVGVGKSALTIQLIQNHFVDEYDPTIEDSYRKQVVIDGETCLL |
| | | DILDTAGQEEYSAMRDQYMRTGEGFLCVFAINNTKSFEDIHQYREQIKRVKDS |
| | | DDVPMVLVGNKCDLPARTVETRQAQDLARSYGIPYIETSAKTRQGVEDAFYTL |
| | | VREIRQHKLRKLNPPDESGPGCMNCKCVIS* |
| 61 | Chicken FGF2 | ATGGCGGCAGGAGCAGCTGGTTCCATAACCACATTGCCCGCCCTCCCAGAC |
| | or FGF2-WT | GATGGGGGCGGAGGCGCATTTCCTCCAGGTCATTTCAAGGACCCCAAAAGG |
| | | CTGTACTGTAAGAATGGTGGTTTTTTCCTGAGGATAAACCCTGACGGACGG |
| | | GTGGACGGCGTACGCGAGAAATCAGATCCACATATCAAGCTGCAGTTGCAA |
| | | GCCGAGGAACGCGGGGTAGTCTCTATAAAAGGGGTTAGCGCTAACAGATTT |
| | | TTGGCAATGAAGGAAGACGGTAGGCTCCTCGCGCTTAAGTGTGCCACCGAG |
| | | GAGTGCTTCTTCTTCGAACGGCTCGAATCTAACAACTACAACACGTACCGCT |
| | | CTCGCAAATACTCTGACTGGTACGTCGCACTCAAACGCACTGGGCAGTATA |
| | | AACCGGGGCCAAAGACGGGTCCGGGGCAGAAAGCTATCCTGTTCCTTCCAA |
| | | TGTCCGCCAAGAGTTAG |
| 62 | BM40-FGF2 | ATGGCGGCAGGAGCAGCTGGTTCCATAACCACATTGCCCGCCCTCCCAGAC |
| | | GATGGGGGCGGAGGCGCATTTCCTCCAGGTCATTTCAAGGACCCCAAAAGG |
| | | CTGTACTGTAAGAATGGTGGTTTTTTCCTGAGGATAAACCCTGACGGACGG |
| | | GTGGACGGCGTACGCGAGAAATCAGATCCACATATCAAGCTGCAGTTGCAA |
| | | GCCGAGGAACGCGGGGTAGTCTCTATAAAAGGGGTTAGCGCTAACAGATTT |
| | | TTGGCAATGAAGGAAGACGGTAGGCTCCTCGCGCTTAAGTGTGCCACCGAG |
| | | GAGTGCTTCTTCTTCGAACGGCTCGAATCTAACAACTACAACACGTACCGCT |
| | | CTCGCAAATACTCTGACTGGTACGTCGCACTCAAACGCACTGGGCAGTATA |
| | | AACCGGGGCCAAAGACGGGTCCGGGGCAGAAAGCTATCCTGTTCCTTCCAA |
| | | TGTCCGCCAAGAGTTAG |
| 63 | ggIL2-FGF2 | ATGATGTGTAAAGTTCTTATCTTCGGATGCATCTCCGTAGCAATGCTCATGA |
| | | CTACAGCTTACATGGCGGCAGGAGCAGCTGGTTCCATAACCACATTGCCCG |
| | | CCCTCCCAGACGATGGGGGCGGAGGCGCATTTCCTCCAGGTCATTTCAAGG |
| | | ACCCCAAAAGGCTGTACTGTAAGAATGGTGGTTTTTTCCTGAGGATAAACC |
| | | CTGACGGACGGGTGGACGGCGTACGCGAGAAATCAGATCCACATATCAAGC |
| | | TGCAGTTGCAAGCCGAGGAACGCGGGGTAGTCTCTATAAAAGGGGTTAGCG |
| | | CTAACAGATTTTTGGCAATGAAGGAAGACGGTAGGCTCCTCGCGCTTAAGT |
| | | GTGCCACCGAGGAGTGCTTCTTCTTCGAACGGCTCGAATCTAACAACTACA |
| | | ACACGTACCGCTCTCGCAAATACTCTGACTGGTACGTCGCACTCAAACGCA |
| | | CTGGGCAGTATAAACCGGGGCCAAAGACGGGTCCGGGGCAGAAAGCTATC |
| | | CTGTTCCTTCCAATGTCCGCCAAGAGTTAG |
| 64 | GLuc-FGF2 | ATGGGGGTCAAAGTGCTGTTTGCCTTGATCTGTATTGCTGTGGCCGAGGCAA |
| | | TGGCGGCAGGAGCAGCTGGTTCCATAACCACATTGCCCGCCCTCCCAGACG |
| | | ATGGGGGCGGAGGCGCATTTCCTCCAGGTCATTTCAAGGACCCCAAAAGGC |
| | | TGTACTGTAAGAATGGTGGTTTTTTCCTGAGGATAAACCCTGACGGACGGGT |
| | | GGACGGCGTACGCGAGAAATCAGATCCACATATCAAGCTGCAGTTGCAAGC |
| | | CGAGGAACGCGGGGTAGTCTCTATAAAAGGGGTTAGCGCTAACAGATTTTT |
| | | GGCAATGAAGGAAGACGGTAGGCTCCTCGCGCTTAAGTGTGCCACCGAGGA |
| | | GTGCTTCTTCTTCGAACGGCTCGAATCTAACAACTACAACACGTACCGCTCT |
| | | CGCAAATACTCTGACTGGTACGTCGCACTCAAACGCACTGGGCAGTATAAA |
| | | CCGGGGCCAAAGACGGGTCCGGGGCAGAAAGCTATCCTGTTCCTTCCAATG |
| | | TCCGCCAAGAGTTAG |
| 65 | hIL2-FGF2 | ATGTATCGCATGCAACTGCTTTCATGCATTGCTCTTAGCCTGGCGCTGGTCA |
| | | CGAACTCTATGGCGGCAGGAGCAGCTGGTTCCATAACCACATTGCCCGCCC |
| | | TCCCAGACGATGGGGGCGGAGGCGCATTTCCTCCAGGTCATTTCAAGGACC |
| | | CCAAAAGGCTGTACTGTAAGAATGGTGGTTTTTTCCTGAGGATAAACCCTG |
| | | ACGGACGGGTGGACGGCGTACGCGAGAAATCAGATCCACATATCAAGCTGC |
| | | AGTTGCAAGCCGAGGAACGCGGGGTAGTCTCTATAAAAGGGGTTAGCGCTA |
| | | ACAGATTTTTGGCAATGAAGGAAGACGGTAGGCTCCTCGCGCTTAAGTGTG |
| | | CCACCGAGGAGTGCTTCTTCTTCGAACGGCTCGAATCTAACAACTACAACA |
| | | CGTACCGCTCTCGCAAATACTCTGACTGGTACGTCGCACTCAAACGCACTGG |
| | | GCAGTATAAACCGGGGCCAAAGACGGGTCCGGGGCAGAAAGCTATCCTGTT |
| | | CCTTCCAATGTCCGCCAAGAGTTAG |
| 66 | hIL2co1-FGF2 | ATGAGAATGCAACTGCTCCTGCTTATAGCGCTCAGTTTGGCTCTCGTGACCA |
| | | ACTCAATGGCGGCAGGAGCAGCTGGTTCCATAACCACATTGCCCGCCCTCC |
| | | CAGACGATGGGGGCGGAGGCGCATTTCCTCCAGGTCATTTCAAGGACCCCA |
| | | AAAGGCTGTACTGTAAGAATGGTGGTTTTTTCCTGAGGATAAACCCTGACG |
| | | GACGGGTGGACGGCGTACGCGAGAAATCAGATCCACATATCAAGCTGCAGT |
| | | TGCAAGCCGAGGAACGCGGGGTAGTCTCTATAAAAGGGGTTAGCGCTAACA |
| | | GATTTTTGGCAATGAAGGAAGACGGTAGGCTCCTCGCGCTTAAGTGTGCCA |
| | | CCGAGGAGTGCTTCTTCTTCGAACGGCTCGAATCTAACAACTACAACACGT |
| | | ACCGCTCTCGCAAATACTCTGACTGGTACGTCGCACTCAAACGCACTGGGC |
| | | AGTATAAACCGGGGCCAAAGACGGGTCCGGGGCAGAAAGCTATCCTGTTCC |
| | | TTCCAATGTCCGCCAAGAGTTAG |
| 67 | hIL2co2-FGF2 | ATGAGGCGGATGCAATTGCTGCTGTTGATCGCACTCTCTCTGGCACTTGTCA |
| | | CTAATAGTATGGCGGCAGGAGCAGCTGGTTCCATAACCACATTGCCCGCCC |
| | | TCCCAGACGATGGGGGCGGAGGCGCATTTCCTCCAGGTCATTTCAAGGACC |
| | | CCAAAAGGCTGTACTGTAAGAATGGTGGTTTTTTCCTGAGGATAAACCCTG |
| | | ACGGACGGGTGGACGGCGTACGCGAGAAATCAGATCCACATATCAAGCTGC |
| | | AGTTGCAAGCCGAGGAACGCGGGGTAGTCTCTATAAAAGGGGTTAGCGCTA |
| | | ACAGATTTTTGGCAATGAAGGAAGACGGTAGGCTCCTCGCGCTTAAGTGTG |
| | | CCACCGAGGAGTGCTTCTTCTTCGAACGGCTCGAATCTAACAACTACAACA |
| | | CGTACCGCTCTCGCAAATACTCTGACTGGTACGTCGCACTCAAACGCACTGG |
| | | GCAGTATAAACCGGGGCCAAAGACGGGTCCGGGGCAGAAAGCTATCCTGTT |
| | | CCTTCCAATGTCCGCCAAGAGTTAG |
| 68 | ggIL6-FGF2 | ATGAATTTTACAGAAGGGTGTGAAGCGACTGGCAGGAGACCAGGATCCGCC |
| | | GGGTCAAGGAGAAGAAGGGCCCCCCGGCCTGGGCCTGTCGCGCTTCTTCCC |
| | | CTGTTGCTTCCGCTGTTGCTTCCACCGGCAGCTGCGGTTCCCTTGCCCATGG |
| | | CGGCAGGAGCAGCTGGTTCCATAACCACATTGCCCGCCCTCCCAGACGATG |
| | | GGGGCGGAGGCGCATTTCCTCCAGGTCATTTCAAGGACCCCAAAAGGCTGT |
| | | ACTGTAAGAATGGTGGTTTTTTCCTGAGGATAAACCCTGACGGACGGGTGG |
| | | ACGGCGTACGCGAGAAATCAGATCCACATATCAAGCTGCAGTTGCAAGCCG |
| | | AGGAACGCGGGGTAGTCTCTATAAAAGGGGTTAGCGCTAACAGATTTTTGG |
| | | CAATGAAGGAAGACGGTAGGCTCCTCGCGCTTAAGTGTGCCACCGAGGAGT |
| | | GCTTCTTCTTCGAACGGCTCGAATCTAACAACTACAACACGTACCGCTCTCG |
| | | CAAATACTCTGACTGGTACGTCGCACTCAAACGCACTGGGCAGTATAAACC |
| | | GGGGCCAAAGACGGGTCCGGGGCAGAAAGCTATCCTGTTCCTTCCAATGTC |
| | | CGCCAAGAGTTAG |
| 69 | hIFN2A- | ATGGCGCTCACGTTTGCCCTCTTGGTTGCGCTTCTCGTTCTTTCCTGCAAGAG |
| | ggFGF2 | TAGCTGTTCCGTGGGAATGGTATCCAAGATGGCGGCAGGAGCAGCTGGTTC |
| | | CATAACCACATTGCCCGCCCTCCCAGACGATGGGGGCGGAGGCGCATTTCC |
| | | TCCAGGTCATTTCAAGGACCCCAAAAGGCTGTACTGTAAGAATGGTGGTTTT |
| | | TTCCTGAGGATAAACCCTGACGGACGGGTGGACGGCGTACGCGAGAAATCA |
| | | GATCCACATATCAAGCTGCAGTTGCAAGCCGAGGAACGCGGGGTAGTCTCT |
| | | ATAAAAGGGGTTAGCGCTAACAGATTTTTGGCAATGAAGGAAGACGGTAGG |
| | | CTCCTCGCGCTTAAGTGTGCCACCGAGGAGTGCTTCTTCTTCGAACGGCTCG |
| | | AATCTAACAACTACAACACGTACCGCTCTCGCAAATACTCTGACTGGTACGT |
| | | CGCACTCAAACGCACTGGGCAGTATAAACCGGGGCCAAAGACGGGTCCGG |
| | | GGCAGAAAGCTATCCTGTTCCTTCCAATGTCCGCCAAGAGTTAG |
| 70 | secrecon- | ATGTGGTGGCGCTTGTGGTGGTTGCTGTTGTTGTTGCTGCTGTTGTGGCCTAT |
| | FGF2 | GGTCTGGGCAATGGCGGCAGGAGCAGCTGGTTCCATAACCACATTGCCCGC |
| | | CCTCCCAGACGATGGGGGCGGAGGCGCATTTCCTCCAGGTCATTTCAAGGA |
| | | CCCCAAAAGGCTGTACTGTAAGAATGGTGGTTTTTTCCTGAGGATAAACCCT |
| | | GACGGACGGGTGGACGGCGTACGCGAGAAATCAGATCCACATATCAAGCT |
| | | GCAGTTGCAAGCCGAGGAACGCGGGGTAGTCTCTATAAAAGGGGTTAGCGC |
| | | TAACAGATTTTTGGCAATGAAGGAAGACGGTAGGCTCCTCGCGCTTAAGTG |
| | | TGCCACCGAGGAGTGCTTCTTCTTCGAACGGCTCGAATCTAACAACTACAAC |
| | | ACGTACCGCTCTCGCAAATACTCTGACTGGTACGTCGCACTCAAACGCACTG |
| | | GGCAGTATAAACCGGGGCCAAAGACGGGTCCGGGGCAGAAAGCTATCCTG |
| | | TTCCTTCCAATGTCCGCCAAGAGTTAG |
| 71 | hIL6-FGF2 | ATGAATAGCTTCAGTACGTCTGCGTTCGGACCTGTGGCTTTTAGCCTCGGAC |
| | | TGCTGCTCGTGCTGCCGGCGGCGTTTCCGGCACCCATGGCGGCAGGAGCAG |
| | | CTGGTTCCATAACCACATTGCCCGCCCTCCCAGACGATGGGGGCGGAGGCG |
| | | CATTTCCTCCAGGTCATTTCAAGGACCCCAAAAGGCTGTACTGTAAGAATG |
| | | GTGGTTTTTTCCTGAGGATAAACCCTGACGGACGGGTGGACGGCGTACGCG |
| | | AGAAATCAGATCCACATATCAAGCTGCAGTTGCAAGCCGAGGAACGCGGG |
| | | GTAGTCTCTATAAAAGGGGTTAGCGCTAACAGATTTTTGGCAATGAAGGAA |
| | | GACGGTAGGCTCCTCGCGCTTAAGTGTGCCACCGAGGAGTGCTTCTTCTTCG |
| | | AACGGCTCGAATCTAACAACTACAACACGTACCGCTCTCGCAAATACTCTG |
| | | ACTGGTACGTCGCACTCAAACGCACTGGGCAGTATAAACCGGGGCCAAAGA |
| | | CGGGTCCGGGGCAGAAAGCTATCCTGTTCCTTCCAATGTCCGCCAAGAGTT |
| | | AG |
| 72 | FGF2-Q68I- | ATGGCGGCAGGAGCAGCTGGTTCCATAACCACATTGCCCGCCCTCCCAGAC |
| | N114G-FGF2 | GATGGGGGCGGAGGCGCATTTCCTCCAGGTCATTTCAAGGACCCCAAAAGG |
| | | CTGTACTGTAAGAATGGTGGTTTTTTCCTGAGGATAAACCCTGACGGACGG |
| | | GTGGACGGCGTACGCGAGAAATCAGATCCACATATCAAGCTGCAGTTGATA |
| | | GCCGAGGAACGCGGGGTAGTCTCTATAAAAGGGGTTAGCGCTAACAGATTT |
| | | TTGGCAATGAAGGAAGACGGTAGGCTCCTCGCGCTTAAGTGTGCCACCGAG |
| | | GAGTGCTTCTTCTTCGAACGGCTCGAATCTAACGGCTACAACACGTACCGCT |
| | | CTCGCAAATACTCTGACTGGTACGTCGCACTCAAACGCACTGGGCAGTATA |
| | | AACCGGGGCCAAAGACGGGTCCGGGGCAGAAAGCTATCCTGTTCCTTCCAA |
| | | TGTCCGCCAAGAGTTAG |
| 73 | FGF2-Q68I- | ATGGCGGCAGGAGCAGCTGGTTCCATAACCACATTGCCCGCCCTCCCAGAC |
| | N114G-C99S- | GATGGGGGCGGAGGCGCATTTCCTCCAGGTCATTTCAAGGACCCCAAAAGG |
| | FGF2 | CTGTACTGTAAGAATGGTGGTTTTTTCCTGAGGATAAACCCTGACGGACGG |
| | | GTGGACGGCGTACGCGAGAAATCAGATCCACATATCAAGCTGCAGTTGATA |
| | | GCCGAGGAACGCGGGGTAGTCTCTATAAAAGGGGTTAGCGCTAACAGATTT |
| | | TTGGCAATGAAGGAAGACGGTAGGCTCCTCGCGCTTAAGTCTGCCACCGAG |
| | | GAGTGCTTCTTCTTCGAACGGCTCGAATCTAACGGCTACAACACGTACCGCT |
| | | CTCGCAAATACTCTGACTGGTACGTCGCACTCAAACGCACTGGGCAGTATA |
| | | AACCGGGGCCAAAGACGGGTCCGGGGCAGAAAGCTATCCTGTTCCTTCCAA |
| | | TGTCCGCCAAGAGTTAG |
| 74 | hIL2- | ATGAGAATGCAACTGCTCCTGCTTATAGCGCTCAGTTTGGCTCTCGTGACCA |
| | colggFGF2 | ACTCAGCGGCAGGAGCAGCTGGTTCCATAACCACATTGCCCGCCCTCCCAG |
| | (xMet) | ACGATGGGGGCGGAGGCGCATTTCCTCCAGGTCATTTCAAGGACCCCAAAA |
| | | GGCTGTACTGTAAGAATGGTGGTTTTTTCCTGAGGATAAACCCTGACGGAC |
| | | GGGTGGACGGCGTACGCGAGAAATCAGATCCACATATCAAGCTGCAGTTGC |
| | | AAGCCGAGGAACGCGGGGTAGTCTCTATAAAAGGGGTTAGCGCTAACAGAT |
| | | TTTTGGCAATGAAGGAAGACGGTAGGCTCCTCGCGCTTAAGTGTGCCACCG |
| | | AGGAGTGCTTCTTCTTCGAACGGCTCGAATCTAACAACTACAACACGTACC |
| | | GCTCTCGCAAATACTCTGACTGGTACGTCGCACTCAAACGCACTGGGCAGT |
| | | ATAAACCGGGGCCAAAGACGGGTCCGGGGCAGAAAGCTATCCTGTTCCTTC |
| | | CAATGTCCGCCAAGAGTTAG |
| 75 | FGF2-C77Y81 | ATGGCGGCAGGAGCAGCTGGTTCCATAACCACATTGCCCGCCCTCCCAGAC |
| | | GATGGGGGCGGAGGCGCATTTCCTCCAGGTCATTTCAAGGACCCCAAAAGG |
| | | CTGTACTGTAAGAATGGTGGTTTTTTCCTGAGGATAAACCCTGACGGACGG |
| | | GTGGACGGCGTACGCGAGAAATCAGATCCACATATCAAGCTGCAGTTGCAA |
| | | GCCGAGGAACGCGGGGTAGTCTCTATAAAAGGGGTTTGCGCTAACAGATAT |
| | | TTGGCAATGAAGGAAGACGGTAGGCTCCTCGCGCTTAAGTGTGCCACCGAG |
| | | GAGTGCTTCTTCTTCGAACGGCTCGAATCTAACAACTACAACACGTACCGCT |
| | | CTCGCAAATACTCTGACTGGTACGTCGCACTCAAACGCACTGGGCAGTATA |
| | | AACCGGGGCCAAAGACGGGTCCGGGGCAGAAAGCTATCCTGTTCCTTCCAA |
| | | TGTCCGCCAAGAGTTAG |
| 76 | FGF-STAB | ATGGCGGCAGGAGCAGCTGGTTCCATAACCACATTGCCCGCCCTCCCAGAC |
| | | GATGGGGGCGGAGGCGCATTTCCTCCAGGTCATTTCAAGGACCCCAAAAGG |
| | | CTGTACTGTAAGAATGGTGGTTTTTTCCTGCTGATAAACCCTGACGGACGGG |
| | | TGGACGGCACCCGCGAGAAATCAGATCCATTCATCAAGCTGCAGTTGCAAG |
| | | CCGAGGAACGCGGGGTAGTCTCTATAAAAGGGGTTAGCGCTAACAGATTTT |
| | | TGGCAATGAAGGAAGACGGTAGGCTCTACGCGCTTAAGTATGCCACCGAGG |
| | | AGTGCTTCTTCTTCGAACGGCTCGAAGAGAACAACTACAACACGTACCGCT |
| | | CTCGCAAATACTCTGACTGGTACGTCGCACTCAAACGCACTGGGCAGTATA |
| | | AACCGGGGCCAAAGACGGGTCCGGGGCAGAAAGCTATCCTGTTCCTTCCAA |
| | | TGTCCGCCAAGAGTTAG |
| 77 | ggIL2-FGF- | ATGATGTGTAAAGTTCTTATCTTCGGATGCATCTCCGTAGCAATGCTCATGA |
| | STAB | CTACAGCTTACATGGCGGCAGGAGCAGCTGGTTCCATAACCACATTGCCCG |
| | | CCCTCCCAGACGATGGGGGCGGAGGCGCATTTCCTCCAGGTCATTTCAAGG |
| | | ACCCCAAAAGGCTGTACTGTAAGAATGGTGGTTTTTTCCTGCTGATAAACCC |
| | | TGACGGACGGGTGGACGGCACCCGCGAGAAATCAGATCCATTCATCAAGCT |
| | | GCAGTTGCAAGCCGAGGAACGCGGGGTAGTCTCTATAAAAGGGGTTAGCGC |
| | | TAACAGATTTTTGGCAATGAAGGAAGACGGTAGGCTCTACGCGCTTAAGTA |
| | | TGCCACCGAGGAGTGCTTCTTCTTCGAACGGCTCGAAGAGAACAACTACAA |
| | | CACGTACCGCTCTCGCAAATACTCTGACTGGTACGTCGCACTCAAACGCACT |
| | | GGGCAGTATAAACCGGGGCCAAAGACGGGTCCGGGGCAGAAAGCTATCCT |
| | | GTTCCTTCCAATGTCCGCCAAGAGTTAG |
| 78 | hIL2-FGF- | ATGTATCGCATGCAACTGCTTTCATGCATTGCTCTTAGCCTGGCGCTGGTCA |
| | STAB | CGAACTCTATGGCGGCAGGAGCAGCTGGTTCCATAACCACATTGCCCGCCC |
| | | TCCCAGACGATGGGGGCGGAGGCGCATTTCCTCCAGGTCATTTCAAGGACC |
| | | CCAAAAGGCTGTACTGTAAGAATGGTGGTTTTTTCCTGCTGATAAACCCTGA |
| | | CGGACGGGTGGACGGCACCCGCGAGAAATCAGATCCATTCATCAAGCTGCA |
| | | GTTGCAAGCCGAGGAACGCGGGGTAGTCTCTATAAAAGGGGTTAGCGCTAA |
| | | CAGATTTTTGGCAATGAAGGAAGACGGTAGGCTCTACGCGCTTAAGTATGC |
| | | CACCGAGGAGTGCTTCTTCTTCGAACGGCTCGAAGAGAACAACTACAACAC |
| | | GTACCGCTCTCGCAAATACTCTGACTGGTACGTCGCACTCAAACGCACTGG |
| | | GCAGTATAAACCGGGGCCAAAGACGGGTCCGGGGCAGAAAGCTATCCTGTT |
| | | CCTTCCAATGTCCGCCAAGAGTTAG |
| 79 | STAB- | ATGGCGGCAGGAGCAGCTGGTTCCATAACCACATTGCCCGCCCTCCCAGAC |
| | C77Y81 | GATGGGGGCGGAGGCGCATTTCCTCCAGGTCATTTCAAGGACCCCAAAAGG |
| | | CTGTACTGTAAGAATGGTGGTTTTTTCCTGAGGATAAACCCTGACGGACGG |
| | | GTGGACGGCGTACGCGAGAAATCAGATCCACATATCAAGCTGCAGTTGCAA |
| | | GCCGAGGAACGCGGGGTAGTCTCTATAAAAGGGGTTTGCGCTAACAGATAT |
| | | TTGGCAATGAAGGAAGACGGTAGGCTCCTCGCGCTTAAGTGTGCCACCGAG |
| | | GAGTGCTTCTTCTTCGAACGGCTCGAATCTAACAACTACAACACGTACCGCT |
| | | CTCGCAAATACTCTGACTGGTACGTCGCACTCAAACGCACTGGGCAGTATA |
| | | AACCGGGGCCAAAGACGGGTCCGGGGCAGAAAGCTATCCTGTTCCTTCCAA |
| | | TGTCCGCCAAGAGTTAG |
| 80 | c96-STAB- | ATGGCGGCAGGAGCAGCTGGTTCCATAACCACATTGCCCGCCCTCCCAGAC |
| | Gen2-ggFGF2 | GATGGGGGCGGAGGCGCATTTCCTCCAGGTCATTTCAAGGACCCCAAAAGG |
| | | CTGTACTGTAAGAATGGTGGTTTTTTCCTGCTGATAAACCCTGACGGACGGG |
| | | TGGACGGCACCCGCGAGAAATCAGATCCATTCATCAAGCTGCAGTTGCAAG |
| | | CCGAGGAACGCGGGGTAGTCTCTATAAAAGGGGTTAGCGCTAACAGATTTT |
| | | TGGCAATGAAGGAAGACGGTAGGCTCTACGCGCTTAAGTGTGCCACCGAGG |
| | | AGTGCTTCTTCTTCGAACGGCTCGAAGAGAACAACTACAACACGTACCGCT |
| | | CTCGCAAATACTCTGACTGGTACGTCGCACTCAAACGCACTGGGCAGTATA |
| | | AACCGGGGCCAAAGACGGGTCCGGGGCAGAAAGCTATCCTGTTCCTTCCAA |
| | | TGTCCGCCAAGAGTTAG |
| 81 | STAB-Gen3- | ATGGCGGCAGGAGCAGCTGGTTCCATAACCACATTGCCCGCCCTCCCAGAC |
| | ggFGF2 | GATGGGGGCGGAGGCGCATTTCCTCCAGGTCATTTCAAGGACCCCAAAAGG |
| | | CTGTACTGTAAGAATGGTGGTTTTTTCCTGCTGATAAACCCTGACGGACGGG |
| | | TGGACGGCACCCGCGACAAATCAGATCCATTCATCAAGCTGCAGTTGCAAG |
| | | CCGAGGAACGCGGGGTAGTCTCTATAAAAGGGGTTAGCGCTAACAGATTTT |
| | | TGGCAATGAAGGAAGACGGTAGGCTCTACGCGATAAAGAATGCCACCGAG |
| | | GAGTGCTTCTTCTTCGAACGGCTCGAAGAGAACAACTACAACACGTACCGC |
| | | TCTCGCAAATACCCTGACTGGTACGTCGCACTCAAACGCACTGGGCAGTAT |
| | | AAACCGGGGCCAAAGACGGGTCCGGGGCAGAAAGCTATCCTGTTCCTTCCA |
| | | ATGTCCGCCAAGAGTTAG |
| 82 | c96-STAB- | ATGGCGGCAGGAGCAGCTGGTTCCATAACCACATTGCCCGCCCTCCCAGAC |
| | Gen3-ggFGF2 | GATGGGGGCGGAGGCGCATTTCCTCCAGGTCATTTCAAGGACCCCAAAAGG |
| | | CTGTACTGTAAGAATGGTGGTTTTTTCCTGCTGATAAACCCTGACGGACGGG |
| | | TGGACGGCACCCGCGACAAATCAGATCCATTCATCAAGCTGCAGTTGCAAG |
| | | CCGAGGAACGCGGGGTAGTCTCTATAAAAGGGGTTAGCGCTAACAGATTTT |
| | | TGGCAATGAAGGAAGACGGTAGGCTCTACGCGATAAAGTGTGCCACCGAG |
| | | GAGTGCTTCTTCTTCGAACGGCTCGAAGAGAACAACTACAACACGTACCGC |
| | | TCTCGCAAATACCCTGACTGGTACGTCGCACTCAAACGCACTGGGCAGTAT |
| | | AAACCGGGGCCAAAGACGGGTCCGGGGCAGAAAGCTATCCTGTTCCTTCCA |
| | | ATGTCCGCCAAGAGTTAG |
| 83 | Bovine FGF2 | ATGGCCGCCGGGAGCATCACCACGCTGCCAGCCCTGCCGGAGGACGGCGGC |
| | | AGCGGCGCTTTCCCGCCGGGCCACTTCAAGGACCCCAAGCGGCTGTACTGC |
| | | AAGAACGGGGGCTTCTTCCTGCGCATCCACCCCGACGGCCGAGTGGACGGG |
| | | GTCCGCGAGAAGAGCGACCCACACATCAAACTACAACTTCAAGCAGAAGA |
| | | GAGAGGGGTTGTGTCTATCAAAGGAGTGTGTGCAAACCGTTACCTTGCTAT |
| | | GAAAGAAGATGGAAGATTACTAGCTTCTAAATGTGTTACAGACGAGTGTTT |
| | | CTTTTTTGAACGATTGGAGTCTAATAACTACAATACTTACCGGTCAAGGAAA |
| | | TACTCCAGTTGGTATGTGGCACTGAAACGAACTGGGCAGTATAAACTTGGA |
| | | CCCAAAACAGGACCTGGGCAGAAAGCTATACTTTTTCTTCCAATGTCTGCTA |
| | | AGAGCTGA |
| 84 | Salmon FGF2 | ATGGCCACAGGAGAAATCACCACTCTACCCGCCACACCTGAAGATGGAGGC |
| | | AGTGGCGGCTTCCCTCCAGGAAACTTTAAGGATCCCAAGAGGCTGTACTGT |
| | | AAAAACGGGGGCTACTTCTTGAGAATAAACTCTAATGGAAGCGTGGACGGG |
| | | ATCCGAGAGAAGAACGACCCCCACATCAAGCTTCAACTCCAGGCGACCTCA |
| | | GTAGGGGAGGTAGTGATCAAAGGGGTCTCAGCCAACCGTTATCTGGCCATG |
| | | AATGGAGATGGAAGACTGTTTGGAACGAGACGGACAACAGATGAATGCTA |
| | | CTTCATGGAGAGGCTGGAGAGTAACAACTACAACACCTACCGCTCACGGAA |
| | | GTACCCTGACATGTATGTGGCGCTGAAAAGGACTGGCCAGTACAAGTCAGG |
| | | ATCCAAAACTGGACCGGGCCAAAAAGCCATTCTCTTTCTCCCCATGTCAGCC |
| | | AGACGCTGA |
| 85 | RASv12 | ATGACCGAGTACAAGCTGGTGGTAGTGGGAGCTGTAGGTGTCGGGAAGAGC |
| | | GCTTTGACGATACAGCTCATTCAGAACCATTTTGTTGATGAGTACGACCCCA |
| | | CAATAGAGGATTCCTACAGAAAGCAAGTCGTCATCGATGGAGAGACCTGTT |
| | | TGCTGGACATCCTGGATACGGCGGGGCAGGAGGAGTACAGTGCCATGCGAG |
| | | ACCAGTACATGAGAACGGGGGAAGGATTCCTGTGCGTCTTTGCCATTAACA |
| | | ACACCAAGTCCTTTGAGGACATCCACCAGTACAGGGAGCAGATCAAGAGGG |
| | | TGAAAGACTCAGATGATGTCCCCATGGTGCTGGTGGGAAATAAATGTGATC |
| | | TGCCAGCACGGACAGTGGAGACCCGGCAAGCGCAGGACCTGGCCCGGAGT |
| | | TACGGGATCCCCTACATAGAAACGTCGGCCAAAACCAGACAGGGCGTCGAA |
| | | GATGCCTTCTATACCTTAGTGCGGGAGATCCGTCAGCATAAACTGCGCAAG |
| | | CTGAACCCACCAGATGAGAGTGGCCCTGGCTGCATGAACTGTAAATGCGTG |
| | | ATATCGTGA |
| 86 | FGFR1-WT | ATGTTCACGTGGAGATGTCTCATCCTGTGGGCGGTACTGGTTACCGCAACGT |
| | | TGAGCGCAGCTAGGCCCGCCCCCACGCTGCCTGACCAGGCTCTCCCTAAGG |
| | | CGAATATAGAAGTAGAATCACATTCTGCACATCCAGGAGACTTGTTGCAAT |
| | | TGCGGTGCCGCTTGAGAGACGATGTCCAATCAATCAATTGGGTCAGGGACG |
| | | GGGTCCAACTTCCGGAGAATAACCGGACAAGGATTACTGGAGAGGAGGTTG |
| | | AAGTCAGGGACGCTGTTCCCGAAGATAGCGGCTTGTACGCTTGCATGACTA |
| | | ATTCCCCGTCAGGGTCAGAAACCACGTATTTTTCTGTAAACGTTAGCGATGC |
| | | TCTTCCGAGTGCTGAAGACGATGATGATGAAGACGATAGCAGCTCCGAAGA |
| | | GAAAGAGGCGGATAATACTAAACCAAATCAAGCTGTTGCACCATATTGGAC |
| | | CTACCCCGAAAAAATGGAGAAAAAGTTGCATGCTGTTCCGGCAGCCAAAAC |
| | | CGTAAAATTCAAGTGCCCCTCCGGCGGTACCCCTAATCCAACTCTGAGATG |
| | | GTTGAAGAACGGTAAGGAGTTCAAACCGGATCACCGGATAGGTGGATATAA |
| | | AGTTCGGTATGCGACCTGGTCCATTATTATGGACTCTGTCGTGCCCTCCGAC |
| | | AAAGGTAACTACACTTGTATCGTCGAGAACAAGTACGGCAGCATCAATCAT |
| | | ACGTACCAACTGGACGTGGTAGAACGCAGTCCACACCGCCCCATCCTCCAG |
| | | GCCGGACTCCCTGCCAACAAAACAGTTGCGCTCGGCTCTAATGTTGAATTTG |
| | | TGTGCAAAGTTTACTCAGACCCTCAACCTCATATCCAATGGCTTAAACATAT |
| | | CGAAGTCAACGGTAGTAAGATAGGTCCCGACAACCTGCCGTATGTCCAGAT |
| | | CCTTAAAACTGCGGGGGTAAATACCACTGACAAGGAAATGGAGGTCTTGCA |
| | | TCTTCGCAACGTGAGCTTTGAAGATGCAGGTGAGTATACTTGTTTGGCAGGT |
| | | AATAGCATCGGAATCTCCCACCATTCCGCCTGGCTGACAGTGCTGGAGGCC |
| | | ACGGAACAAAGTCCGGCCATGATGACCAGTCCGTTGTATCTGGAAATTATC |
| | | ATTTACTGTACAGGGGCCTTTCTCATATCATGCATGGTCGTAACCGTGATTA |
| | | TATATAAAATGAAGAGCACAACGAAGAAGACTGATTTTAATTCACAACTGG |
| | | CGGTACATAAACTCGCAAAATCTATCCCATTGAGGCGGCAGGTTACAGTCT |
| | | CCGCCGACAGCTCCAGCAGCATGAACTCAGGAGTGATGCTTGTTCGCCCCA |
| | | GCAGACTGAGTTCTAGTGGGACTCCGATGCTTGCTGGAGTCAGTGAATACG |
| | | AATTGCCGGAGGATCCGCGGTGGGAACTTCCTAGGGACCGCCTTATATTGG |
| | | GCAAACCCCTCGGTGAGGGCTGTTTCGGACAGGTCGTGCTCGCAGAGGCCA |
| | | TCGGCCTTGACAAAGATAAGCCGAATAGAGTGACCAAGGTGGCGGTTAAAA |
| | | TGCTGAAATCAGACGCTACGGAAAAGGACCTCTCAGACCTCATCAGTGAAA |
| | | TGGAAATGATGAAAATGATAGGGAAGCACAAAAACATCATCAATTTGCTCG |
| | | GAGCTTGTACCCAGGACGGTCCCCTCTACGTGATCGTAGAATACGCTTCCAA |
| | | AGGTAATCTGCGCGAATATCTGCAAGCTCGGAGGCCGCCAGGTATGGAATA |
| | | CTGTTATAATCCGACACGGATTCCCGAGGAACAGCTCTCTTTTAAAGATTTG |
| | | GTTTCATGCGCGTATCAAGTGGCGAGGGGCATGGAGTATCTGGCGTCCAAG |
| | | AAGTGCATTCATAGGGACTTGGCTGCAAGAAATGTCTTGGTAACAGAAGAC |
| | | AACGTCATGAAGATCGCCGACTTCGGCCTTGCACGGGATATTCATCACATC |
| | | GACTATTACAAAAAGACGACGAACGGCCGCCTCCCAGTTAAGTGGATGGCC |
| | | CCCGAAGCCCTGTTCGATCGGATTTACACGCATCAATCCGACGTGTGGTCTT |
| | | TCGGTGTCCTGCTTTGGGAGATATTTACACTCGGGGGATCACCCTACCCCGG |
| | | AGTACCGGTGGAGGAGTTGTTCAAACTTCTTAAAGAAGGTCACAGAATGGA |
| | | CAAACCCAGTAACTGCACTAACGAGCTGTATATGATGATGCGCGATTGCTG |
| | | GCACGCTGTTCCGTCACAACGGCCCACTTTTAAACAGCTCGTGGAGGATCTT |
| | | GACAGAATCGTCGCGATGACTAGCAACCAAGAGTATTTGGATTTGTCAGTC |
| | | CCGCTTGACCAATATTCCCCCGGTTTTCCGGCTACCCGCTCTTCTACTTGTTC |
| | | CAGCGGTGAGGATAGTGTATTTTCTCATGACCCACTTCCAGATGAGCCGTGC |
| | | TTGCCTCGGTGTCCTCCCCACTCCCATGGAGCGCTCAAACGCCACTGA |
| 87 | FGFR1C- | ATGTTCACGTGGAGATGTCTCATCCTGTGGGCGGTACTGGTTACCGCAACGT |
| | N546K | TGAGCGCAGCTAGGCCCGCCCCCACGCTGCCTGACCAGGCTCTCCCTAAGG |
| | | CGAATATAGAAGTAGAATCACATTCTGCACATCCAGGAGACTTGTTGCAAT |
| | | TGCGGTGCCGCTTGAGAGACGATGTCCAATCAATCAATTGGGTCAGGGACG |
| | | GGGTCCAACTTCCGGAGAATAACCGGACAAGGATTACTGGAGAGGAGGTTG |
| | | AAGTCAGGGACGCTGTTCCCGAAGATAGCGGCTTGTACGCTTGCATGACTA |
| | | ATTCCCCGTCAGGGTCAGAAACCACGTATTTTTCTGTAAACGTTAGCGATGC |
| | | TCTTCCGAGTGCTGAAGACGATGATGATGAAGACGATAGCAGCTCCGAAGA |
| | | GAAAGAGGCGGATAATACTAAACCAAATCAAGCTGTTGCACCATATTGGAC |
| | | CTACCCCGAAAAAATGGAGAAAAAGTTGCATGCTGTTCCGGCAGCCAAAAC |
| | | CGTAAAATTCAAGTGCCCCTCCGGCGGTACCCCTAATCCAACTCTGAGATG |
| | | GTTGAAGAACGGTAAGGAGTTCAAACCGGATCACCGGATAGGTGGATATAA |
| | | AGTTCGGTATGCGACCTGGTCCATTATTATGGACTCTGTCGTGCCCTCCGAC |
| | | AAAGGTAACTACACTTGTATCGTCGAGAACAAGTACGGCAGCATCAATCAT |
| | | ACGTACCAACTGGACGTGGTAGAACGCAGTCCACACCGCCCCATCCTCCAG |
| | | GCCGGACTCCCTGCCAACAAAACAGTTGCGCTCGGCTCTAATGTTGAATTTG |
| | | TGTGCAAAGTTTACTCAGACCCTCAACCTCATATCCAATGGCTTAAACATAT |
| | | CGAAGTCAACGGTAGTAAGATAGGTCCCGACAACCTGCCGTATGTCCAGAT |
| | | CCTTAAAACTGCGGGGGTAAATACCACTGACAAGGAAATGGAGGTCTTGCA |
| | | TCTTCGCAACGTGAGCTTTGAAGATGCAGGTGAGTATACTTGTTTGGCAGGT |
| | | AATAGCATCGGAATCTCCCACCATTCCGCCTGGCTGACAGTGCTGGAGGCC |
| | | ACGGAACAAAGTCCGGCCATGATGACCAGTCCGTTGTATCTGGAAATTATC |
| | | ATTTACTGTACAGGGGCCTTTCTCATATCATGCATGGTCGTAACCGTGATTA |
| | | TATATAAAATGAAGAGCACAACGAAGAAGACTGATTTTAATTCACAACTGG |
| | | CGGTACATAAACTCGCAAAATCTATCCCATTGAGGCGGCAGGTTACAGTCT |
| | | CCGCCGACAGCTCCAGCAGCATGAACTCAGGAGTGATGCTTGTTCGCCCCA |
| | | GCAGACTGAGTTCTAGTGGGACTCCGATGCTTGCTGGAGTCAGTGAATACG |
| | | AATTGCCGGAGGATCCGCGGTGGGAACTTCCTAGGGACCGCCTTATATTGG |
| | | GCAAACCCCTCGGTGAGGGCTGTTTCGGACAGGTCGTGCTCGCAGAGGCCA |
| | | TCGGCCTTGACAAAGATAAGCCGAATAGAGTGACCAAGGTGGCGGTTAAAA |
| | | TGCTGAAATCAGACGCTACGGAAAAGGACCTCTCAGACCTCATCAGTGAAA |
| | | TGGAAATGATGAAAATGATAGGGAAGCACAAAAACATCATCAAGTTGCTCG |
| | | GAGCTTGTACCCAGGACGGTCCCCTCTACGTGATCGTAGAATACGCTTCCAA |
| | | AGGTAATCTGCGCGAATATCTGCAAGCTCGGAGGCCGCCAGGTATGGAATA |
| | | CTGTTATAATCCGACACGGATTCCCGAGGAACAGCTCTCTTTTAAAGATTTG |
| | | GTTTCATGCGCGTATCAAGTGGCGAGGGGCATGGAGTATCTGGCGTCCAAG |
| | | AAGTGCATTCATAGGGACTTGGCTGCAAGAAATGTCTTGGTAACAGAAGAC |
| | | AACGTCATGAAGATCGCCGACTTCGGCCTTGCACGGGATATTCATCACATC |
| | | GACTATTACAAAAAGACGACGAACGGCCGCCTCCCAGTTAAGTGGATGGCC |
| | | CCCGAAGCCCTGTTCGATCGGATTTACACGCATCAATCCGACGTGTGGTCTT |
| | | TCGGTGTCCTGCTTTGGGAGATATTTACACTCGGGGGATCACCCTACCCCGG |
| | | AGTACCGGTGGAGGAGTTGTTCAAACTTCTTAAAGAAGGTCACAGAATGGA |
| | | CAAACCCAGTAACTGCACTAACGAGCTGTATATGATGATGCGCGATTGCTG |
| | | GCACGCTGTTCCGTCACAACGGCCCACTTTTAAACAGCTCGTGGAGGATCTT |
| | | GACAGAATCGTCGCGATGACTAGCAACCAAGAGTATTTGGATTTGTCAGTC |
| | | CCGCTTGACCAATATTCCCCCGGTTTTCCGGCTACCCGCTCTTCTACTTGTTC |
| | | CAGCGGTGAGGATAGTGTATTTTCTCATGACCCACTTCCAGATGAGCCGTGC |
| | | TTGCCTCGGTGTCCTCCCCACTCCCATGGAGCGCTCAAACGCCACTGA |
| 88 | FGFR1C- | GCCGCCACCATGTTCACGTGGAGATGTCTCATCCTGTGGGCGGTACTGGTTA |
| | V561M | CCGCAACGTTGAGCGCAGCTAGGCCCGCCCCCACGCTGCCTGACCAGGCTC |
| | | TCCCTAAGGCGAATATAGAAGTAGAATCACATTCTGCACATCCAGGAGACT |
| | | TGTTGCAATTGCGGTGCCGCTTGAGAGACGATGTCCAATCAATCAATTGGGT |
| | | CAGGGACGGGGTCCAACTTCCGGAGAATAACCGGACAAGGATTACTGGAG |
| | | AGGAGGTTGAAGTCAGGGACGCTGTTCCCGAAGATAGCGGCTTGTACGCTT |
| | | GCATGACTAATTCCCCGTCAGGGTCAGAAACCACGTATTTTTCTGTAAACGT |
| | | TAGCGATGCTCTTCCGAGTGCTGAAGACGATGATGATGAAGACGATAGCAG |
| | | CTCCGAAGAGAAAGAGGCGGATAATACTAAACCAAATCAAGCTGTTGCACC |
| | | ATATTGGACCTACCCCGAAAAAATGGAGAAAAAGTTGCATGCTGTTCCGGC |
| | | AGCCAAAACCGTAAAATTCAAGTGCCCCTCCGGCGGTACCCCTAATCCAAC |
| | | TCTGAGATGGTTGAAGAACGGTAAGGAGTTCAAACCGGATCACCGGATAGG |
| | | TGGATATAAAGTTCGGTATGCGACCTGGTCCATTATTATGGACTCTGTCGTG |
| | | CCCTCCGACAAAGGTAACTACACTTGTATCGTCGAGAACAAGTACGGCAGC |
| | | ATCAATCATACGTACCAACTGGACGTGGTAGAACGCAGTCCACACCGCCCC |
| | | ATCCTCCAGGCCGGACTCCCTGCCAACAAAACAGTTGCGCTCGGCTCTAAT |
| | | GTTGAATTTGTGTGCAAAGTTTACTCAGACCCTCAACCTCATATCCAATGGC |
| | | TTAAACATATCGAAGTCAACGGTAGTAAGATAGGTCCCGACAACCTGCCGT |
| | | ATGTCCAGATCCTTAAAACTGCGGGGGTAAATACCACTGACAAGGAAATGG |
| | | AGGTCTTGCATCTTCGCAACGTGAGCTTTGAAGATGCAGGTGAGTATACTTG |
| | | TTTGGCAGGTAATAGCATCGGAATCTCCCACCATTCCGCCTGGCTGACAGTG |
| | | CTGGAGGCCACGGAACAAAGTCCGGCCATGATGACCAGTCCGTTGTATCTG |
| | | GAAATTATCATTTACTGTACAGGGGCCTTTCTCATATCATGCATGGTCGTAA |
| | | CCGTGATTATATATAAAATGAAGAGCACAACGAAGAAGACTGATTTTAATT |
| | | CACAACTGGCGGTACATAAACTCGCAAAATCTATCCCATTGAGGCGGCAGG |
| | | TTACAGTCTCCGCCGACAGCTCCAGCAGCATGAACTCAGGAGTGATGCTTG |
| | | TTCGCCCCAGCAGACTGAGTTCTAGTGGGACTCCGATGCTTGCTGGAGTCAG |
| | | TGAATACGAATTGCCGGAGGATCCGCGGTGGGAACTTCCTAGGGACCGCCT |
| | | TATATTGGGCAAACCCCTCGGTGAGGGCTGTTTCGGACAGGTCGTGCTCGC |
| | | AGAGGCCATCGGCCTTGACAAAGATAAGCCGAATAGAGTGACCAAGGTGG |
| | | CGGTTAAAATGCTGAAATCAGACGCTACGGAAAAGGACCTCTCAGACCTCA |
| | | TCAGTGAAATGGAAATGATGAAAATGATAGGGAAGCACAAAAACATCATC |
| | | AATTTGCTCGGAGCTTGTACCCAGGACGGTCCCCTCTACGTGATCATGGAAT |
| | | ACGCTTCCAAAGGTAATCTGCGCGAATATCTGCAAGCTCGGAGGCCGCCAG |
| | | GTATGGAATACTGTTATAATCCGACACGGATTCCCGAGGAACAGCTCTCTTT |
| | | TAAAGATTTGGTTTCATGCGCGTATCAAGTGGCGAGGGGCATGGAGTATCT |
| | | GGCGTCCAAGAAGTGCATTCATAGGGACTTGGCTGCAAGAAATGTCTTGGT |
| | | AACAGAAGACAACGTCATGAAGATCGCCGACTTCGGCCTTGCACGGGATAT |
| | | TCATCACATCGACTATTACAAAAAGACGACGAACGGCCGCCTCCCAGTTAA |
| | | GTGGATGGCCCCCGAAGCCCTGTTCGATCGGATTTACACGCATCAATCCGA |
| | | CGTGTGGTCTTTCGGTGTCCTGCTTTGGGAGATATTTACACTCGGGGGATCA |
| | | CCCTACCCCGGAGTACCGGTGGAGGAGTTGTTCAAACTTCTTAAAGAAGGT |
| | | CACAGAATGGACAAACCCAGTAACTGCACTAACGAGCTGTATATGATGATG |
| | | CGCGATTGCTGGCACGCTGTTCCGTCACAACGGCCCACTTTTAAACAGCTCG |
| | | TGGAGGATCTTGACAGAATCGTCGCGATGACTAGCAACCAAGAGTATTTGG |
| | | ATTTGTCAGTCCCGCTTGACCAATATTCCCCCGGTTTTCCGGCTACCCGCTCT |
| | | TCTACTTGTTCCAGCGGTGAGGATAGTGTATTTTCTCATGACCCACTTCCAG |
| | | ATGAGCCGTGCTTGCCTCGGTGTCCTCCCCACTCCCATGGAGCGCTCAAACG |
| | | CCACTGA |
| 89 | FGFR1C- | ATGTTCACGTGGAGATGTCTCATCCTGTGGGCGGTACTGGTTACCGCAACGT |
| | K656E | TGAGCGCAGCTAGGCCCGCCCCCACGCTGCCTGACCAGGCTCTCCCTAAGG |
| | | CGAATATAGAAGTAGAATCACATTCTGCACATCCAGGAGACTTGTTGCAAT |
| | | TGCGGTGCCGCTTGAGAGACGATGTCCAATCAATCAATTGGGTCAGGGACG |
| | | GGGTCCAACTTCCGGAGAATAACCGGACAAGGATTACTGGAGAGGAGGTTG |
| | | AAGTCAGGGACGCTGTTCCCGAAGATAGCGGCTTGTACGCTTGCATGACTA |
| | | ATTCCCCGTCAGGGTCAGAAACCACGTATTTTTCTGTAAACGTTAGCGATGC |
| | | TCTTCCGAGTGCTGAAGACGATGATGATGAAGACGATAGCAGCTCCGAAGA |
| | | GAAAGAGGCGGATAATACTAAACCAAATCAAGCTGTTGCACCATATTGGAC |
| | | CTACCCCGAAAAAATGGAGAAAAAGTTGCATGCTGTTCCGGCAGCCAAAAC |
| | | CGTAAAATTCAAGTGCCCCTCCGGCGGTACCCCTAATCCAACTCTGAGATG |
| | | GTTGAAGAACGGTAAGGAGTTCAAACCGGATCACCGGATAGGTGGATATAA |
| | | AGTTCGGTATGCGACCTGGTCCATTATTATGGACTCTGTCGTGCCCTCCGAC |
| | | AAAGGTAACTACACTTGTATCGTCGAGAACAAGTACGGCAGCATCAATCAT |
| | | ACGTACCAACTGGACGTGGTAGAACGCAGTCCACACCGCCCCATCCTCCAG |
| | | GCCGGACTCCCTGCCAACAAAACAGTTGCGCTCGGCTCTAATGTTGAATTTG |
| | | TGTGCAAAGTTTACTCAGACCCTCAACCTCATATCCAATGGCTTAAACATAT |
| | | CGAAGTCAACGGTAGTAAGATAGGTCCCGACAACCTGCCGTATGTCCAGAT |
| | | CCTTAAAACTGCGGGGGTAAATACCACTGACAAGGAAATGGAGGTCTTGCA |
| | | TCTTCGCAACGTGAGCTTTGAAGATGCAGGTGAGTATACTTGTTTGGCAGGT |
| | | AATAGCATCGGAATCTCCCACCATTCCGCCTGGCTGACAGTGCTGGAGGCC |
| | | ACGGAACAAAGTCCGGCCATGATGACCAGTCCGTTGTATCTGGAAATTATC |
| | | ATTTACTGTACAGGGGCCTTTCTCATATCATGCATGGTCGTAACCGTGATTA |
| | | TATATAAAATGAAGAGCACAACGAAGAAGACTGATTTTAATTCACAACTGG |
| | | CGGTACATAAACTCGCAAAATCTATCCCATTGAGGCGGCAGGTTACAGTCT |
| | | CCGCCGACAGCTCCAGCAGCATGAACTCAGGAGTGATGCTTGTTCGCCCCA |
| | | GCAGACTGAGTTCTAGTGGGACTCCGATGCTTGCTGGAGTCAGTGAATACG |
| | | AATTGCCGGAGGATCCGCGGTGGGAACTTCCTAGGGACCGCCTTATATTGG |
| | | GCAAACCCCTCGGTGAGGGCTGTTTCGGACAGGTCGTGCTCGCAGAGGCCA |
| | | TCGGCCTTGACAAAGATAAGCCGAATAGAGTGACCAAGGTGGCGGTTAAAA |
| | | TGCTGAAATCAGACGCTACGGAAAAGGACCTCTCAGACCTCATCAGTGAAA |
| | | TGGAAATGATGAAAATGATAGGGAAGCACAAAAACATCATCAATTTGCTCG |
| | | GAGCTTGTACCCAGGACGGTCCCCTCTACGTGATCGTAGAATACGCTTCCAA |
| | | AGGTAATCTGCGCGAATATCTGCAAGCTCGGAGGCCGCCAGGTATGGAATA |
| | | CTGTTATAATCCGACACGGATTCCCGAGGAACAGCTCTCTTTTAAAGATTTG |
| | | GTTTCATGCGCGTATCAAGTGGCGAGGGGCATGGAGTATCTGGCGTCCAAG |
| | | AAGTGCATTCATAGGGACTTGGCTGCAAGAAATGTCTTGGTAACAGAAGAC |
| | | AACGTCATGAAGATCGCCGACTTCGGCCTTGCACGGGATATTCATCACATC |
| | | GACTATTACAAAGAGACGACGAACGGCCGCCTCCCAGTTAAGTGGATGGCC |
| | | CCCGAAGCCCTGTTCGATCGGATTTACACGCATCAATCCGACGTGTGGTCTT |
| | | TCGGTGTCCTGCTTTGGGAGATATTTACACTCGGGGGATCACCCTACCCCGG |
| | | AGTACCGGTGGAGGAGTTGTTCAAACTTCTTAAAGAAGGTCACAGAATGGA |
| | | CAAACCCAGTAACTGCACTAACGAGCTGTATATGATGATGCGCGATTGCTG |
| | | GCACGCTGTTCCGTCACAACGGCCCACTTTTAAACAGCTCGTGGAGGATCTT |
| | | GACAGAATCGTCGCGATGACTAGCAACCAAGAGTATTTGGATTTGTCAGTC |
| | | CCGCTTGACCAATATTCCCCCGGTTTTCCGGCTACCCGCTCTTCTACTTGTTC |
| | | CAGCGGTGAGGATAGTGTATTTTCTCATGACCCACTTCCAGATGAGCCGTGC |
| | | TTGCCTCGGTGTCCTCCCCACTCCCATGGAGCGCTCAAACGCCACTGA |
| 90 | FGFR1- | ATGTTCACGTGGAGATGTCTCATCCTGTGGGCGGTACTGGTTACCGCAACGT |
| | N546K- | TGAGCGCAGCTAGGCCCGCCCCCACGCTGCCTGACCAGGCTCTCCCTAAGG |
| | K656E-S780A | CGAATATAGAAGTAGAATCACATTCTGCACATCCAGGAGACTTGTTGCAAT |
| | | TGCGGTGCCGCTTGAGAGACGATGTCCAATCAATCAATTGGGTCAGGGACG |
| | | GGGTCCAACTTCCGGAGAATAACCGGACAAGGATTACTGGAGAGGAGGTTG |
| | | AAGTCAGGGACGCTGTTCCCGAAGATAGCGGCTTGTACGCTTGCATGACTA |
| | | ATTCCCCGTCAGGGTCAGAAACCACGTATTTTTCTGTAAACGTTAGCGATGC |
| | | TCTTCCGAGTGCTGAAGACGATGATGATGAAGACGATAGCAGCTCCGAAGA |
| | | GAAAGAGGCGGATAATACTAAACCAAATCAAGCTGTTGCACCATATTGGAC |
| | | CTACCCCGAAAAAATGGAGAAAAAGTTGCATGCTGTTCCGGCAGCCAAAAC |
| | | CGTAAAATTCAAGTGCCCCTCCGGCGGTACTCCTAATCCAACTCTGAGATGG |
| | | TTGAAGAACGGTAAGGAGTTCAAACCGGATCACCGGATAGGTGGATATAAA |
| | | GTTCGGTATGCGACCTGGTCCATTATTATGGACTCTGTCGTGCCCTCCGACA |
| | | AAGGTAACTACACTTGTATCGTCGAGAACAAGTACGGCAGCATCAATCATA |
| | | CGTACCAACTGGACGTGGTAGAACGCAGTCCACACCGCCCCATCCTCCAGG |
| | | CCGGACTCCCTGCCAACAAAACAGTTGCGCTCGGCTCTAATGTTGAATTTGT |
| | | GTGCAAAGTTTACTCAGACCCTCAACCTCATATCCAATGGCTTAAACATATC |
| | | GAAGTCAACGGTAGTAAGATAGGTCCCGACAACCTGCCGTATGTCCAGATC |
| | | CTTAAAACTGCGGGGGTAAATACCACTGACAAGGAAATGGAGGTCTTGCAT |
| | | CTTCGCAACGTGAGCTTTGAAGATGCAGGTGAGTATACTTGTTTGGCAGGTA |
| | | ATAGCATCGGAATCTCCCACCATTCCGCCTGGCTGACAGTGCTGGAGGCCA |
| | | CGGAACAAAGTCCGGCCATGATGACCAGTCCGTTGTATCTGGAAATTATCA |
| | | TTTACTGTACAGGGGCCTTTCTCATATCATGCATGGTCGTAACCGTGATTAT |
| | | ATATAAAATGAAGAGCACAACGAAGAAGACTGATTTTAATTCACAACTGGC |
| | | GGTACATAAACTCGCAAAATCTATCCCATTGAGGCGGCAGGTTACAGTCTC |
| | | CGCCGACAGCTCCAGCAGCATGAACTCAGGAGTGATGCTTGTTCGCCCCAG |
| | | CAGACTGAGTTCTAGTGGGACTCCGATGCTTGCTGGAGTCAGTGAATACGA |
| | | ATTGCCGGAGGATCCGCGGTGGGAACTTCCTAGGGACCGCCTTATATTGGG |
| | | CAAACCCCTCGGTGAGGGCTGTTTCGGACAGGTCGTGCTCGCAGAGGCCAT |
| | | CGGCCTTGACAAAGATAAGCCGAATAGAGTGACCAAGGTGGCGGTTAAAAT |
| | | GCTGAAATCAGACGCTACGGAAAAGGACCTCTCAGACCTCATCAGTGAAAT |
| | | GGAAATGATGAAAATGATAGGGAAGCACAAAAACATCATCAAGTTGCTCG |
| | | GAGCTTGTACCCAGGACGGTCCCCTCTACGTGATCGTAGAATACGCTTCCAA |
| | | AGGTAATCTGCGCGAATATCTGCAAGCTCGGAGGCCGCCAGGTATGGAATA |
| | | CTGTTATAATCCGACACGGATTCCCGAGGAACAGCTCTCTTTTAAAGATTTG |
| | | GTTTCATGCGCGTATCAAGTGGCGAGGGGCATGGAGTATCTGGCGTCCAAG |
| | | AAGTGCATTCATAGGGACTTGGCTGCAAGAAATGTCTTGGTAACAGAAGAC |
| | | AACGTCATGAAGATCGCCGACTTCGGCCTTGCACGGGATATTCATCACATC |
| | | GACTATTACAAAGAGACGACGAACGGCCGCCTCCCAGTTAAGTGGATGGCC |
| | | CCCGAAGCCCTGTTCGATCGGATTTACACGCATCAATCCGACGTGTGGTCTT |
| | | TCGGTGTCCTGCTTTGGGAGATATTTACACTCGGGGGATCACCCTACCCCGG |
| | | AGTACCGGTGGAGGAGTTGTTCAAACTTCTTAAAGAAGGTCACAGAATGGA |
| | | CAAACCCAGTAACTGCACTAACGAGCTGTATATGATGATGCGCGATTGCTG |
| | | GCACGCTGTTCCGTCACAACGGCCCACTTTTAAACAGCTCGTGGAGGATCTT |
| | | GACAGAATCGTCGCGATGACTAGCAACCAAGAGTATTTGGATTTGTCAGTC |
| | | CCGCTTGACCAATATGCCCCCGGTTTTCCGGCTACCCGCTCTTCTACTTGTTC |
| | | CAGCGGTGAGGATAGTGTATTTTCTCATGACCCACTTCCAGATGAGCCGTGC |
| | | TTGCCTCGGTGTCCTCCCCACTCCCATGGAGCGCTCAAACGCCACTGA |
| 91 | myrist- | ATGGGATCATCCAAGTCAAAACCGAAAGACCCGTCACAGAGAAAAATGAA |
| | FGFR1C- | GAGCACAACGAAGAAGACTGATTTTAATTCACAACTGGCGGTACATAAACT |
| | K656E | CGCAAAATCTATCCCATTGAGGCGGCAGGTTACAGTCTCCGCCGACAGCTC |
| | | CAGCAGCATGAACTCAGGAGTGATGCTTGTTCGCCCCAGCAGACTGAGTTC |
| | | TAGTGGGACTCCGATGCTTGCTGGAGTCAGTGAATACGAATTGCCGGAGGA |
| | | TCCGCGGTGGGAACTTCCTAGGGACCGCCTTATATTGGGCAAACCCCTCGGT |
| | | GAGGGCTGTTTCGGACAGGTCGTGCTCGCAGAGGCCATCGGCCTTGACAAA |
| | | GATAAGCCGAATAGAGTGACCAAGGTGGCGGTTAAAATGCTGAAATCAGA |
| | | CGCTACGGAAAAGGACCTCTCAGACCTCATCAGTGAAATGGAAATGATGAA |
| | | AATGATAGGGAAGCACAAAAACATCATCAATTTGCTCGGAGCTTGTACCCA |
| | | GGACGGTCCCCTCTACGTGATCGTAGAATACGCTTCCAAAGGTAATCTGCG |
| | | CGAATATCTGCAAGCTCGGAGGCCGCCAGGTATGGAATACTGTTATAATCC |
| | | GACACGGATTCCCGAGGAACAGCTCTCTTTTAAAGATTTGGTTTCATGCGCG |
| | | TATCAAGTGGCGAGGGGCATGGAGTATCTGGCGTCCAAGAAGTGCATTCAT |
| | | AGGGACTTGGCTGCAAGAAATGTCTTGGTAACAGAAGACAACGTCATGAAG |
| | | ATCGCCGACTTCGGCCTTGCACGGGATATTCATCACATCGACTATTACAAAG |
| | | AGACGACGAACGGCCGCCTCCCAGTTAAGTGGATGGCCCCCGAAGCCCTGT |
| | | TCGATCGGATTTACACGCATCAATCCGACGTGTGGTCTTTCGGTGTCCTGCT |
| | | TTGGGAGATATTTACACTCGGGGGATCACCCTACCCCGGAGTACCGGTGGA |
| | | GGAGTTGTTCAAACTTCTTAAAGAAGGTCACAGAATGGACAAACCCAGTAA |
| | | CTGCACTAACGAGCTGTATATGATGATGCGCGATTGCTGGCACGCTGTTCCG |
| | | TCACAACGGCCCACTTTTAAACAGCTCGTGGAGGATCTTGACAGAATCGTC |
| | | GCGATGACTAGCAACCAAGAGTATTTGGATTTGTCAGTCCCGCTTGACCAAT |
| | | ATTCCCCCGGTTTTCCGGCTACCCGCTCTTCTACTTGTTCCAGCGGTGAGGA |
| | | TAGTGTATTTTCTCATGACCCACTTCCAGATGAGCCGTGCTTGCCTCGGTGT |
| | | CCTCCCCACTCCCATGGAGCGCTCAAACGCCACTGA |
| 92 | FGFR2-WT | ATGGGCCTTAAGTCAACTTGGAGATACGGCAATGGCCCGGGTACGTACTCC |
| | | AAGAAAATGGTATCTTGGGATTCCGGTTGTCTCATTTGTCTGGTAGTGGTTA |
| | | CCATGGCGGGCCTGAGTCTGGCGAGACCCTCTTTTAATCTGGTAGTTGAAGA |
| | | CGCTACCTTGGAGCCAGAAGAGCCGCCCACTAAGTATCAGATAAGTCAGCC |
| | | TGATGTGCACTCCGCGCTTCCGGGAGAACCTCTCGAGTTGCGCTGTCAACTC |
| | | AAGGATGCCGTGATGATCAGCTGGACGAAAGATGGAGTGCCTCTTGGACCA |
| | | GATAATCGCACTGTTATCATTGGTGAATACTTGCAAATTAAAGATGCATCAC |
| | | CACGGGATTCTGGCCTTTACGCGTGCACAGCTATCAGGACGCTCGACTCCG |
| | | ATACACTCTATTTTATAGTCAACGTTACCGATGCGTTGTCAAGCGGGGATGA |
| | | TGAGGACGACAACGACGGGTCAGAAGACTTCGTTAACGATTCCAATCAGAT |
| | | GAGAGCTCCCTATTGGACCCACACTGACAAAATGGAAAAAAGGCTCCACGC |
| | | TGTCCCGGCTGCGAATACTGTGAAGTTTAGATGTCCCGCAATGGGCAACCC |
| | | CACGCCAACAATGCGGTGGTTGAAAAATGGTAAAGAGTTCAAACAGGAGC |
| | | ATCGGATAGGTGGGTATAAAGTGAGAAATCAACATTGGAGCTTGATCATGG |
| | | AGTCTGTAGTTCCTTCAGATAAAGGAAATTATACATGCATCGTCGAGAATC |
| | | AATACGGCTCTATTAACCATACATATCACCTGGACGTCGTAGAGAGGAGTC |
| | | CCCACAGGCCTATCCTGCAAGCGGGACTGCCGGCAAATGCTTCTGCAGTCG |
| | | TGGGCGGTGACGTTGAGTTCGTCTGTAAGGTGTACAGCGACGCACAACCAC |
| | | ACATCCAATGGATAAAGCATGTTGAGAGAAATGGTTCAAAATATGGCCCGG |
| | | ACGGCTTGCCCTACCTTCAGGTGCTCAAAGCTGCGGGAGTCAACACTACTG |
| | | ATAAGGAAATCGAGGTGCTCTATATTAGGAACGTTACCTTTGAAGACGCCG |
| | | GCGAATACACCTGTCTCGCGGGAAACTCTATCGGTATCTCATTTCACACCGC |
| | | ATGGTTGACTGTGCTTCCAGCTCCGGAGAAAGAGAAGGAGTTTCCGACCTC |
| | | CCCTGATTACCTCGAAATAGCGATCTATTGCATCGGAGTTTTTCTCATCGCG |
| | | TGCATGGTTCTTACTGTGATACTTTGTAGAATGAAGAACACCACCAAGAAA |
| | | CCGGATTTTTCCTCCCAACCGGCCGTCCACAAGTTGACGAAACGGATCCCAT |
| | | TGAGGCGCCAGGTGAGTGCTGACAGCTCAAGTTCAATGAACAGTAACACGC |
| | | CGCTCGTGAGGATCACTACTCGGCTGAGCTCTACCGCGGATGCGCCAATGTT |
| | | GGCAGGGGTCAGTGAGTACGAACTCCCGGAAGATCCGAAGTGGGAGTTTCC |
| | | GCGCGACAAACTCACTCTTGGAAAACCGCTTGGAGAGGGATGCTTCGGACA |
| | | GGTCGTAATGGCCGAGGCGGTTGGTATAGACAAAGATAGACCCAAAGAAG |
| | | CTGTCACAGTAGCTGTAAAAATGCTTAAGGATGATGCCACTGAAAAAGATT |
| | | TGAGCGACCTCGTAAGCGAGATGGAAATGATGAAAATGATAGGGAAGCAC |
| | | AAAAATATAATTAACCTCCTGGGGGCCTGCACACAGGACGGCCCGTTGTAT |
| | | GTCATCGTCGAATATGCCTCCAAAGGGAACCTCAGGGAGTACCTTAGAGCG |
| | | CGCAGACCGCCGGGGATGGAGTATTCATTTGACATCAATCGGGTCCCCGAA |
| | | GAACAAATGACTTTCAAAGACCTTGTCTCCTGTACCTATCAACTCGCCCGCG |
| | | GAATGGAATATTTGGCTAGTCAAAAATGCATTCACCGCGATCTTGCTGCAC |
| | | GGAACGTACTCGTCACTGAGAATAACGTTATGAAAATAGCGGATTTCGGCC |
| | | TCGCAAGGGACATAAACAACATCGACTACTACAAAAAAACCACGAATGGC |
| | | AGACTGCCAGTCAAGTGGATGGCGCCAGAAGCCCTTTTTGATAGAGTCTAC |
| | | ACGCACCAGTCAGACGTGTGGTCCTTTGGAGTGCTCATGTGGGAAATCTTTA |
| | | CGCTGGGTGGTAGCCCTTACCCGGGGATTCCCGTGGAAGAACTTTTCAAGCT |
| | | GTTGAAAGAGGGCCATCGGATGGACAAACCCGCAAATTGCACAAATGAATT |
| | | GTATATGATGATGCGCGACTGTTGGCAAGCCGTGCCTTCACAGAGACCTAC |
| | | ATTCAAGCAGTTGGTCGAAGACCTCGACCGGATCCTGACGCTTACAACGAA |
| | | CGAAGAATACCTGGACTTGTCTGGTCCCTTGGAGCAATACTCACCAAGCTA |
| | | CCCCGATACTCGGTCATCTTGCTCTAGTGGCGACGATAGTGTCTTTTCACCT |
| | | GATCCAATGCCCTACGAACCGTGTCTGCCAAAGTACCAACACATGAACGGT |
| | | TCAGTAAAGACCTGA |
| 93 | FGFR2- | ATGGGCCTTAAGTCAACTTGGAGATACGGCAATGGCCCGGGTACGTACTCC |
| | N550K | AAGAAAATGGTATCTTGGGATTCCGGTTGTCTCATTTGTCTGGTAGTGGTTA |
| | | CCATGGCGGGCCTGAGTCTGGCGAGACCCTCTTTTAATCTGGTAGTTGAAGA |
| | | CGCTACCTTGGAGCCAGAAGAGCCGCCCACTAAGTATCAGATAAGTCAGCC |
| | | TGATGTGCACTCCGCGCTTCCGGGAGAACCTCTCGAGTTGCGCTGTCAACTC |
| | | AAGGATGCCGTGATGATCAGCTGGACGAAAGATGGAGTGCCTCTTGGACCA |
| | | GATAATCGCACTGTTATCATTGGTGAATACTTGCAAATTAAAGATGCATCAC |
| | | CACGGGATTCTGGCCTTTACGCGTGCACAGCTATCAGGACGCTCGACTCCG |
| | | ATACACTCTATTTTATAGTCAACGTTACCGATGCGTTGTCAAGCGGGGATGA |
| | | TGAGGACGACAACGACGGGTCAGAAGACTTCGTTAACGATTCCAATCAGAT |
| | | GAGAGCTCCCTATTGGACCCACACTGACAAAATGGAAAAAAGGCTCCACGC |
| | | TGTCCCGGCTGCGAATACTGTGAAGTTTAGATGTCCCGCAATGGGCAACCC |
| | | CACGCCAACAATGCGGTGGTTGAAAAATGGTAAAGAGTTCAAACAGGAGC |
| | | ATCGGATAGGTGGGTATAAAGTGAGAAATCAACATTGGAGCTTGATCATGG |
| | | AGTCTGTAGTTCCTTCAGATAAAGGAAATTATACATGCATCGTCGAGAATC |
| | | AATACGGCTCTATTAACCATACATATCACCTGGACGTCGTAGAGAGGAGTC |
| | | CCCACAGGCCTATCCTGCAAGCGGGACTGCCGGCAAATGCTTCTGCAGTCG |
| | | TGGGCGGTGACGTTGAGTTCGTCTGTAAGGTGTACAGCGACGCACAACCAC |
| | | ACATCCAATGGATAAAGCATGTTGAGAGAAATGGTTCAAAATATGGCCCGG |
| | | ACGGCTTGCCCTACCTTCAGGTGCTCAAAGCTGCGGGAGTCAACACTACTG |
| | | ATAAGGAAATCGAGGTGCTCTATATTAGGAACGTTACCTTTGAAGACGCCG |
| | | GCGAATACACCTGTCTCGCGGGAAACTCTATCGGTATCTCATTTCACACCGC |
| | | ATGGTTGACTGTGCTTCCAGCTCCGGAGAAAGAGAAGGAGTTTCCGACCTC |
| | | CCCTGATTACCTCGAAATAGCGATCTATTGCATCGGAGTTTTTCTCATCGCG |
| | | TGCATGGTTCTTACTGTGATACTTTGTAGAATGAAGAACACCACCAAGAAA |
| | | CCGGATTTTTCCTCCCAACCGGCCGTCCACAAGTTGACGAAACGGATCCCAT |
| | | TGAGGCGCCAGGTGAGTGCTGACAGCTCAAGTTCAATGAACAGTAACACGC |
| | | CGCTCGTGAGGATCACTACTCGGCTGAGCTCTACCGCGGATGCGCCAATGTT |
| | | GGCAGGGGTCAGTGAGTACGAACTCCCGGAAGATCCGAAGTGGGAGTTTCC |
| | | GCGCGACAAACTCACTCTTGGAAAACCGCTTGGAGAGGGATGCTTCGGACA |
| | | GGTCGTAATGGCCGAGGCGGTTGGTATAGACAAAGATAGACCCAAAGAAG |
| | | CTGTCACAGTAGCTGTAAAAATGCTTAAGGATGATGCCACTGAAAAAGATT |
| | | TGAGCGACCTCGTAAGCGAGATGGAAATGATGAAAATGATAGGGAAGCAC |
| | | AAAAATATAATTAAGCTCCTGGGGGCCTGCACACAGGACGGCCCGTTGTAT |
| | | GTCATCGTCGAATATGCCTCCAAAGGGAACCTCAGGGAGTACCTTAGAGCG |
| | | CGCAGACCGCCGGGGATGGAGTATTCATTTGACATCAATCGGGTCCCCGAA |
| | | GAACAAATGACTTTCAAAGACCTTGTCTCCTGTACCTATCAACTCGCCCGCG |
| | | GAATGGAATATTTGGCTAGTCAAAAATGCATTCACCGCGATCTTGCTGCAC |
| | | GGAACGTACTCGTCACTGAGAATAACGTTATGAAAATAGCGGATTTCGGCC |
| | | TCGCAAGGGACATAAACAACATCGACTACTACAAAAAAACCACGAATGGC |
| | | AGACTGCCAGTCAAGTGGATGGCGCCAGAAGCCCTTTTTGATAGAGTCTAC |
| | | ACGCACCAGTCAGACGTGTGGTCCTTTGGAGTGCTCATGTGGGAAATCTTTA |
| | | CGCTGGGTGGTAGCCCTTACCCGGGGATTCCCGTGGAAGAACTTTTCAAGCT |
| | | GTTGAAAGAGGGCCATCGGATGGACAAACCCGCAAATTGCACAAATGAATT |
| | | GTATATGATGATGCGCGACTGTTGGCAAGCCGTGCCTTCACAGAGACCTAC |
| | | ATTCAAGCAGTTGGTCGAAGACCTCGACCGGATCCTGACGCTTACAACGAA |
| | | CGAAGAATACCTGGACTTGTCTGGTCCCTTGGAGCAATACTCACCAAGCTA |
| | | CCCCGATACTCGGTCATCTTGCTCTAGTGGCGACGATAGTGTCTTTTCACCT |
| | | GATCCAATGCCCTACGAACCGTGTCTGCCAAAGTACCAACACATGAACGGT |
| | | TCAGTAAAGACCTGA |
| 94 | FGFR2- | ATGGGCCTTAAGTCAACTTGGAGATACGGCAATGGCCCGGGTACGTACTCC |
| | K660E | AAGAAAATGGTATCTTGGGATTCCGGTTGTCTCATTTGTCTGGTAGTGGTTA |
| | | CCATGGCGGGCCTGAGTCTGGCGAGACCCTCTTTTAATCTGGTAGTTGAAGA |
| | | CGCTACCTTGGAGCCAGAAGAGCCGCCCACTAAGTATCAGATAAGTCAGCC |
| | | TGATGTGCACTCCGCGCTTCCGGGAGAACCTCTCGAGTTGCGCTGTCAACTC |
| | | AAGGATGCCGTGATGATCAGCTGGACGAAAGATGGAGTGCCTCTTGGACCA |
| | | GATAATCGCACTGTTATCATTGGTGAATACTTGCAAATTAAAGATGCATCAC |
| | | CACGGGATTCTGGCCTTTACGCGTGCACAGCTATCAGGACGCTCGACTCCG |
| | | ATACACTCTATTTTATAGTCAACGTTACCGATGCGTTGTCAAGCGGGGATGA |
| | | TGAGGACGACAACGACGGGTCAGAAGACTTCGTTAACGATTCCAATCAGAT |
| | | GAGAGCTCCCTATTGGACCCACACTGACAAAATGGAAAAAAGGCTCCACGC |
| | | TGTCCCGGCTGCGAATACTGTGAAGTTTAGATGTCCCGCAATGGGCAACCC |
| | | CACGCCAACAATGCGGTGGTTGAAAAATGGTAAAGAGTTCAAACAGGAGC |
| | | ATCGGATAGGTGGGTATAAAGTGAGAAATCAACATTGGAGCTTGATCATGG |
| | | AGTCTGTAGTTCCTTCAGATAAAGGAAATTATACATGCATCGTCGAGAATC |
| | | AATACGGCTCTATTAACCATACATATCACCTGGACGTCGTAGAGAGGAGTC |
| | | CCCACAGGCCTATCCTGCAAGCGGGACTGCCGGCAAATGCTTCTGCAGTCG |
| | | TGGGCGGTGACGTTGAGTTCGTCTGTAAGGTGTACAGCGACGCACAACCAC |
| | | ACATCCAATGGATAAAGCATGTTGAGAGAAATGGTTCAAAATATGGCCCGG |
| | | ACGGCTTGCCCTACCTTCAGGTGCTCAAAGCTGCGGGAGTCAACACTACTG |
| | | ATAAGGAAATCGAGGTGCTCTATATTAGGAACGTTACCTTTGAAGACGCCG |
| | | GCGAATACACCTGTCTCGCGGGAAACTCTATCGGTATCTCATTTCACACCGC |
| | | ATGGTTGACTGTGCTTCCAGCTCCGGAGAAAGAGAAGGAGTTTCCGACCTC |
| | | CCCTGATTACCTCGAAATAGCGATCTATTGCATCGGAGTTTTTCTCATCGCG |
| | | TGCATGGTTCTTACTGTGATACTTTGTAGAATGAAGAACACCACCAAGAAA |
| | | CCGGATTTTTCCTCCCAACCGGCCGTCCACAAGTTGACGAAACGGATCCCAT |
| | | TGAGGCGCCAGGTGAGTGCTGACAGCTCAAGTTCAATGAACAGTAACACGC |
| | | CGCTCGTGAGGATCACTACTCGGCTGAGCTCTACCGCGGATGCGCCAATGTT |
| | | GGCAGGGGTCAGTGAGTACGAACTCCCGGAAGATCCGAAGTGGGAGTTTCC |
| | | GCGCGACAAACTCACTCTTGGAAAACCGCTTGGAGAGGGATGCTTCGGACA |
| | | GGTCGTAATGGCCGAGGCGGTTGGTATAGACAAAGATAGACCCAAAGAAG |
| | | CTGTCACAGTAGCTGTAAAAATGCTTAAGGATGATGCCACTGAAAAAGATT |
| | | TGAGCGACCTCGTAAGCGAGATGGAAATGATGAAAATGATAGGGAAGCAC |
| | | AAAAATATAATTAACCTCCTGGGGGCCTGCACACAGGACGGCCCGTTGTAT |
| | | GTCATCGTCGAATATGCCTCCAAAGGGAACCTCAGGGAGTACCTTAGAGCG |
| | | CGCAGACCGCCGGGGATGGAGTATTCATTTGACATCAATCGGGTCCCCGAA |
| | | GAACAAATGACTTTCAAAGACCTTGTCTCCTGTACCTATCAACTCGCCCGCG |
| | | GAATGGAATATTTGGCTAGTCAAAAATGCATTCACCGCGATCTTGCTGCAC |
| | | GGAACGTACTCGTCACTGAGAATAACGTTATGAAAATAGCGGATTTCGGCC |
| | | TCGCAAGGGACATAAACAACATCGACTACTACAAAGAAACCACGAATGGC |
| | | AGACTGCCAGTCAAGTGGATGGCGCCAGAAGCCCTTTTTGATAGAGTCTAC |
| | | ACGCACCAGTCAGACGTGTGGTCCTTTGGAGTGCTCATGTGGGAAATCTTTA |
| | | CGCTGGGTGGTAGCCCTTACCCGGGGATTCCCGTGGAAGAACTTTTCAAGCT |
| | | GTTGAAAGAGGGCCATCGGATGGACAAACCCGCAAATTGCACAAATGAATT |
| | | GTATATGATGATGCGCGACTGTTGGCAAGCCGTGCCTTCACAGAGACCTAC |
| | | ATTCAAGCAGTTGGTCGAAGACCTCGACCGGATCCTGACGCTTACAACGAA |
| | | CGAAGAATACCTGGACTTGTCTGGTCCCTTGGAGCAATACTCACCAAGCTA |
| | | CCCCGATACTCGGTCATCTTGCTCTAGTGGCGACGATAGTGTCTTTTCACCT |
| | | GATCCAATGCCCTACGAACCGTGTCTGCCAAAGTACCAACACATGAACGGT |
| | | TCAGTAAAGACCTGA |
| 95 | FGFR2- | ATGGGCCTTAAGTCAACTTGGAGATACGGCAATGGCCCGGGTACGTACTCC |
| | N550K- | AAGAAAATGGTATCTTGGGATTCCGGTTGTCTCATTTGTCTGGTAGTGGTTA |
| | N660E-S780A | CCATGGCGGGCCTGAGTCTGGCGAGACCCTCTTTTAATCTGGTAGTTGAAGA |
| | | CGCTACCTTGGAGCCAGAAGAGCCGCCCACTAAGTATCAGATAAGTCAGCC |
| | | TGATGTGCACTCCGCGCTTCCGGGAGAACCTCTCGAGTTGCGCTGTCAACTC |
| | | AAGGATGCCGTGATGATCAGCTGGACGAAAGATGGAGTGCCTCTTGGACCA |
| | | GATAATCGCACTGTTATCATTGGTGAATACTTGCAAATTAAAGATGCATCAC |
| | | CACGGGATTCTGGCCTTTACGCGTGCACAGCTATCAGGACGCTCGACTCCG |
| | | ATACACTCTATTTTATAGTCAACGTTACCGATGCGTTGTCAAGCGGGGATGA |
| | | TGAGGACGACAACGACGGGTCAGAAGACTTCGTTAACGATTCCAATCAGAT |
| | | GAGAGCTCCCTATTGGACCCACACTGACAAAATGGAAAAAAGGCTCCACGC |
| | | TGTCCCGGCTGCGAATACTGTGAAGTTTAGATGTCCCGCAATGGGCAACCC |
| | | CACGCCAACAATGCGGTGGTTGAAAAATGGTAAAGAGTTCAAACAGGAGC |
| | | ATCGGATAGGTGGGTATAAAGTGAGAAATCAACATTGGAGCTTGATCATGG |
| | | AGTCTGTAGTTCCTTCAGATAAAGGAAATTATACATGCATCGTCGAGAATC |
| | | AATACGGCTCTATTAACCATACATATCACCTGGACGTCGTAGAGAGGAGTC |
| | | CCCACAGGCCTATCCTGCAAGCGGGACTGCCGGCAAATGCTTCTGCAGTCG |
| | | TGGGCGGTGACGTTGAGTTCGTCTGTAAGGTGTACAGCGACGCACAACCAC |
| | | ACATCCAATGGATAAAGCATGTTGAGAGAAATGGTTCAAAATATGGCCCGG |
| | | ACGGCTTGCCCTACCTTCAGGTGCTCAAAGCTGCGGGAGTCAACACTACTG |
| | | ATAAGGAAATCGAGGTGCTCTATATTAGGAACGTTACCTTTGAAGACGCCG |
| | | GCGAATACACCTGTCTCGCGGGAAACTCTATCGGTATCTCATTTCACACCGC |
| | | ATGGTTGACTGTGCTTCCAGCTCCGGAGAAAGAGAAGGAGTTTCCGACCTC |
| | | CCCTGATTACCTCGAAATAGCGATCTATTGCATCGGAGTTTTTCTCATCGCG |
| | | TGCATGGTTCTTACTGTGATACTTTGTAGAATGAAGAACACCACCAAGAAA |
| | | CCGGATTTTTCCTCCCAACCGGCCGTCCACAAGTTGACGAAACGGATCCCAT |
| | | TGAGGCGCCAGGTGAGTGCTGACAGCTCAAGTTCAATGAACAGTAACACGC |
| | | CGCTCGTGAGGATCACTACTCGGCTGAGCTCTACCGCGGATGCGCCAATGTT |
| | | GGCAGGGGTCAGTGAGTACGAACTCCCGGAAGATCCGAAGTGGGAGTTTCC |
| | | GCGCGACAAACTCACTCTTGGAAAACCGCTTGGAGAGGGATGCTTCGGACA |
| | | GGTCGTAATGGCCGAGGCGGTTGGTATAGACAAAGATAGACCCAAAGAAG |
| | | CTGTCACAGTAGCTGTAAAAATGCTTAAGGATGATGCCACTGAAAAAGATT |
| | | TGAGCGACCTCGTAAGCGAGATGGAAATGATGAAAATGATAGGGAAGCAC |
| | | AAAAATATAATTAAGCTCCTGGGGGCCTGCACACAGGACGGCCCGTTGTAT |
| | | GTCATCGTCGAATATGCCTCCAAAGGGAACCTCAGGGAGTACCTTAGAGCG |
| | | CGCAGACCGCCGGGGATGGAGTATTCATTTGACATCAATCGGGTCCCCGAA |
| | | GAACAAATGACTTTCAAAGACCTTGTCTCCTGTACCTATCAACTCGCCCGCG |
| | | GAATGGAATATTTGGCTAGTCAAAAATGCATTCACCGCGATCTTGCTGCAC |
| | | GGAACGTACTCGTCACTGAGAATAACGTTATGAAAATAGCGGATTTCGGCC |
| | | TCGCAAGGGACATAAACAACATCGACTACTACAAAGAAACCACGAATGGC |
| | | AGACTGCCAGTCAAGTGGATGGCGCCAGAAGCCCTTTTTGATAGAGTCTAC |
| | | ACGCACCAGTCAGACGTGTGGTCCTTTGGAGTGCTCATGTGGGAAATCTTTA |
| | | CGCTGGGTGGTAGCCCTTACCCGGGGATTCCCGTGGAAGAACTTTTCAAGCT |
| | | GTTGAAAGAGGGCCATCGGATGGACAAACCCGCAAATTGCACAAATGAATT |
| | | GTATATGATGATGCGCGACTGTTGGCAAGCCGTGCCTTCACAGAGACCTAC |
| | | ATTCAAGCAGTTGGTCGAAGACCTCGACCGGATCCTGACGCTTACAACGAA |
| | | CGAAGAATACCTGGACTTGTCTGGTCCCTTGGAGCAATACGCACCAAGCTA |
| | | CCCCGATACTCGGTCATCTTGCTCTAGTGGCGACGATAGTGTCTTTTCACCT |
| | | GATCCAATGCCCTACGAACCGTGTCTGCCAAAGTACCAACACATGAACGGT |
| | | TCAGTAAAGACCTGA |
| 96 | FGFR3-WT | ATGTCAGAAGCTGGTGGCGGCGCCGCTGCCGCAGCCTCACTCCCGAGATCA |
| | | AGGGCCGGTGGCATGCGCGCGGCATGGGGATCCGTGTGGTGTTTGTGCCTC |
| | | GCGGCGGCTGTCGGAGCTCTGCCGGCTGCAAGGCGCAGGGGAGCGGAACG |
| | | GAGCGGTGGCCAGGCGGCGGAGTATCTCCGGAGTGAAACTGCATTTCTTGA |
| | | GGAGCTTGTTTTCGGTTCAGGGGATACCATCGAGCTTTCCTGTAACACACAA |
| | | TCTTCAAGTGTAAGCGTATTCTGGTTCAAAGATGGTATAGGCATTGCGCCCA |
| | | GTAATAGAACACACATCGGGCAGAAACTTTTGAAGATCATCAACGTTTCAT |
| | | ATGATGATTCCGGACTGTACTCATGTAAGCCAAGGCACAGTAACGAGGTGC |
| | | TTGGGAACTTCACAGTCAGGGTTACAGGCGTACCATTCTGGACGAGGCCCG |
| | | ATAAGATGGAAAAAAAACTTTTGGCAGTGCCCGCAGCCAATACAGTGCGGT |
| | | TCCGCTGCCCCGCAGGTGGGAATCCGACGCCCACAATTTATTGGCTGAAGA |
| | | ATGGCAAGGAGTTCAAAGGAGAACATAGGATCGGCGGAATCAAACTGAGG |
| | | CACCAGCAATGGTCTCTGGTGATGGAATCAGTAGTCCCATCTGACCGCGGT |
| | | AACTACACGTGCGTAGTAGAAAACAAGTATGGGAATATTCGGCATACCTAC |
| | | CAGCTCGATGTGCTCGAGAGATCTCCTCACAGGCCTATCTTGCAGGCTGGAC |
| | | TGCCAGCTAATCAAACAGTAGTTGTCGGATCTAACGTGGAGTTCCACTGCA |
| | | AAGTTTACTCAGACGCCCAACCGCACATTCAATGGCTTAAACACGTGGAGG |
| | | TTAACGGTAGCAAGTATGGACCTGATGGGACTCCCTATGTGACAGTCCTTA |
| | | AGAGTTGGATTTCTAAGAATGCCGAGGCGGATGCGAACTTGAACCTGTTTA |
| | | ACGTAACCGAGCAGGACGAGGGCGAATATCTTTGTCGCGCTAACAACTTCG |
| | | TTGGAATAGCGGAGAAGCCCTTCTGGCTGCATATACGGAAGCCAAAGCCGG |
| | | CGGAGGAGTTGATGGAGATGGATGACTCCGGTTCCGTATACGCCGGAATCC |
| | | TTAGCTACGGAACCGGGCTGGTGCTGTTTATCCTCGTTTTGGTTATTGTTATT |
| | | ATATGCCGCATGAAGATGCCAAACAAAAAGGCAATGAATACTACAACTGTG |
| | | CAGAAAGTGTCAAAATTCCCACTTAAACGGCAAGTCACTGTCTCACTCGAA |
| | | TCTAACTCTTCCATGAACTCCAACACTCCGTTGGTGCGGATTACCAGATTGT |
| | | CCTCATCCGACGGTCCCATGTTGGCAAATGTCAGTGAGCTGGAATTGCCGCC |
| | | CGATCCCAAATGGGAACTTGCAAGATCCCGGCTTACTCTCGGTAAGCCCCT |
| | | GGGCGAAGGTTGCTTTGGACAAGTCGTCATGGCCGAAGCGATTGGAATCGA |
| | | TAAAGACAAACCCAACAAGGCTATAACGGTCGCAGTAAAAATGTTGAAAG |
| | | ATGACGCGACCGACAAGGACCTGTCAGATCTGGTCTCAGAGATGGAAATGA |
| | | TGAAGATGATTGGTAAACACAAGAATATAATCAACTTGCTTGGAGCATGTA |
| | | CGCAAGACGGCCCTCTCTACGTTCTCGTGGAGTATGCCTCTAAGGGCAATTT |
| | | GAGGGAGTACCTGAGAGCTCGCAGACCTCCGGGCATGGATTATAGTTTCGA |
| | | CACTTGTAAGCTGCCGGAGGAGCAACTTACCTTCAAGGATCTTGTAAGTTGC |
| | | GCGTACCAGGTTGCAAGGGGGATGGAATACCTCGCCAGTCAAAAGTGCATA |
| | | CATAGAGATTTGGCAGCGAGGAACGTGCTCGTGACTGAGGACAACGTCATG |
| | | AAAATTGCGGACTTTGGGCTTGCACGGGATGTTCATAACATCGATTACTACA |
| | | AGAAAACTACTAACGGACGGCTCCCTGTGAAGTGGATGGCTCCAGAAGCGC |
| | | TTTTTGATAGAGTTTACACACATCAAAGTGACGTATGGAGTTTTGGAGTATT |
| | | GCTCTGGGAGATTTTCACCTTGGGAGGGTCTCCTTACCCTGGAATACCCGTC |
| | | GAAGAGCTCTTTAAGCTGCTTAAAGAAGGCCACAGGATGGACAAGCCGGCG |
| | | AACTGCACTCATGATCTGTACATGATAATGAGAGAGTGCTGGCACGCCGTT |
| | | CCCTCCCAGCGGCCGACATTCAAGCAGCTGGTGGAAGACCTTGATAGGGTA |
| | | CTCACTATGACGTCCACCGATGAGTATCTGGACTTGTCTGTTCCATTCGAAC |
| | | AATACTCTCCGGCCGGTCAGGATACGCATTCCACTTGCTCCAGTGGTGATGA |
| | | TTCTGTCTTTGCACACGATCTTCTCCCAGACGAACCCTGTCTTCCTAAACAC |
| | | GTACCGTGTAACGGTGTCATAAGGACATAG |
| 97 | FGFR3- | ATGTCAGAAGCTGGTGGCGGCGCCGCTGCCGCAGCCTCACTCCCGAGATCA |
| | N540K | AGGGCCGGTGGCATGCGCGCGGCATGGGGATCCGTGTGGTGTTTGTGCCTC |
| | | GCGGCGGCTGTCGGAGCTCTGCCGGCTGCAAGGCGCAGGGGAGCGGAACG |
| | | GAGCGGTGGCCAGGCGGCGGAGTATCTCCGGAGTGAAACTGCATTTCTTGA |
| | | GGAGCTTGTTTTCGGTTCAGGGGATACCATCGAGCTTTCCTGTAACACACAA |
| | | TCTTCAAGTGTAAGCGTATTCTGGTTCAAAGATGGTATAGGCATTGCGCCCA |
| | | GTAATAGAACACACATCGGGCAGAAACTTTTGAAGATCATCAACGTTTCAT |
| | | ATGATGATTCCGGACTGTACTCATGTAAGCCAAGGCACAGTAACGAGGTGC |
| | | TTGGGAACTTCACAGTCAGGGTTACAGGCGTACCATTCTGGACGAGGCCCG |
| | | ATAAGATGGAAAAAAAACTTTTGGCAGTGCCCGCAGCCAATACAGTGCGGT |
| | | TCCGCTGCCCCGCAGGTGGGAATCCGACGCCCACAATTTATTGGCTGAAGA |
| | | ATGGCAAGGAGTTCAAAGGAGAACATAGGATCGGCGGAATCAAACTGAGG |
| | | CACCAGCAATGGTCTCTGGTGATGGAATCAGTAGTCCCATCTGACCGCGGT |
| | | AACTACACGTGCGTAGTAGAAAACAAGTATGGGAATATTCGGCATACCTAC |
| | | CAGCTCGATGTGCTCGAGAGATCTCCTCACAGGCCTATCTTGCAGGCTGGAC |
| | | TGCCAGCTAATCAAACAGTAGTTGTCGGATCTAACGTGGAGTTCCACTGCA |
| | | AAGTTTACTCAGACGCCCAACCGCACATTCAATGGCTTAAACACGTGGAGG |
| | | TTAACGGTAGCAAGTATGGACCTGATGGGACTCCCTATGTGACAGTCCTTA |
| | | AGAGTTGGATTTCTAAGAATGCCGAGGCGGATGCGAACTTGAACCTGTTTA |
| | | ACGTAACCGAGCAGGACGAGGGCGAATATCTTTGTCGCGCTAACAACTTCG |
| | | TTGGAATAGCGGAGAAGCCCTTCTGGCTGCATATACGGAAGCCAAAGCCGG |
| | | CGGAGGAGTTGATGGAGATGGATGACTCCGGTTCCGTATACGCCGGAATCC |
| | | TTAGCTACGGAACCGGGCTGGTGCTGTTTATCCTCGTTTTGGTTATTGTTATT |
| | | ATATGCCGCATGAAGATGCCAAACAAAAAGGCAATGAATACTACAACTGTG |
| | | CAGAAAGTGTCAAAATTCCCACTTAAACGGCAAGTCACTGTCTCACTCGAA |
| | | TCTAACTCTTCCATGAACTCCAACACTCCGTTGGTGCGGATTACCAGATTGT |
| | | CCTCATCCGACGGTCCCATGTTGGCAAATGTCAGTGAGCTGGAATTGCCGCC |
| | | CGATCCCAAATGGGAACTTGCAAGATCCCGGCTTACTCTCGGTAAGCCCCT |
| | | GGGCGAAGGTTGCTTTGGACAAGTCGTCATGGCCGAAGCGATTGGAATCGA |
| | | TAAAGACAAACCCAACAAGGCTATAACGGTCGCAGTAAAAATGTTGAAAG |
| | | ATGACGCGACCGACAAGGACCTGTCAGATCTGGTCTCAGAGATGGAAATGA |
| | | TGAAGATGATTGGTAAACACAAGAATATAATCAAGTTGCTTGGAGCATGTA |
| | | CGCAAGACGGCCCTCTCTACGTTCTCGTGGAGTATGCCTCTAAGGGCAATTT |
| | | GAGGGAGTACCTGAGAGCTCGCAGACCTCCGGGCATGGATTATAGTTTCGA |
| | | CACTTGTAAGCTGCCGGAGGAGCAACTTACCTTCAAGGATCTTGTAAGTTGC |
| | | GCGTACCAGGTTGCAAGGGGGATGGAATACCTCGCCAGTCAAAAGTGCATA |
| | | CATAGAGATTTGGCAGCGAGGAACGTGCTCGTGACTGAGGACAACGTCATG |
| | | AAAATTGCGGACTTTGGGCTTGCACGGGATGTTCATAACATCGATTACTACA |
| | | AGAAAACTACTAACGGACGGCTCCCTGTGAAGTGGATGGCTCCAGAAGCGC |
| | | TTTTTGATAGAGTTTACACACATCAAAGTGACGTATGGAGTTTTGGAGTATT |
| | | GCTCTGGGAGATTTTCACCTTGGGAGGGTCTCCTTACCCTGGAATACCCGTC |
| | | GAAGAGCTCTTTAAGCTGCTTAAAGAAGGCCACAGGATGGACAAGCCGGCG |
| | | AACTGCACTCATGATCTGTACATGATAATGAGAGAGTGCTGGCACGCCGTT |
| | | CCCTCCCAGCGGCCGACATTCAAGCAGCTGGTGGAAGACCTTGATAGGGTA |
| | | CTCACTATGACGTCCACCGATGAGTATCTGGACTTGTCTGTTCCATTCGAAC |
| | | AATACTCTCCGGCCGGTCAGGATACGCATTCCACTTGCTCCAGTGGTGATGA |
| | | TTCTGTCTTTGCACACGATCTTCTCCCAGACGAACCCTGTCTTCCTAAACAC |
| | | GTACCGTGTAACGGTGTCATAAGGACATAG |
| 98 | FGFR3- | ATGTCAGAAGCTGGTGGCGGCGCCGCTGCCGCAGCCTCACTCCCGAGATCA |
| | K650E | AGGGCCGGTGGCATGCGCGCGGCATGGGGATCCGTGTGGTGTTTGTGCCTC |
| | | GCGGCGGCTGTCGGAGCTCTGCCGGCTGCAAGGCGCAGGGGAGCGGAACG |
| | | GAGCGGTGGCCAGGCGGCGGAGTATCTCCGGAGTGAAACTGCATTTCTTGA |
| | | GGAGCTTGTTTTCGGTTCAGGGGATACCATCGAGCTTTCCTGTAACACACAA |
| | | TCTTCAAGTGTAAGCGTATTCTGGTTCAAAGATGGTATAGGCATTGCGCCCA |
| | | GTAATAGAACACACATCGGGCAGAAACTTTTGAAGATCATCAACGTTTCAT |
| | | ATGATGATTCCGGACTGTACTCATGTAAGCCAAGGCACAGTAACGAGGTGC |
| | | TTGGGAACTTCACAGTCAGGGTTACAGGCGTACCATTCTGGACGAGGCCCG |
| | | ATAAGATGGAAAAAAAACTTTTGGCAGTGCCCGCAGCCAATACAGTGCGGT |
| | | TCCGCTGCCCCGCAGGTGGGAATCCGACGCCCACAATTTATTGGCTGAAGA |
| | | ATGGCAAGGAGTTCAAAGGAGAACATAGGATCGGCGGAATCAAACTGAGG |
| | | CACCAGCAATGGTCTCTGGTGATGGAATCAGTAGTCCCATCTGACCGCGGT |
| | | AACTACACGTGCGTAGTAGAAAACAAGTATGGGAATATTCGGCATACCTAC |
| | | CAGCTCGATGTGCTCGAGAGATCTCCTCACAGGCCTATCTTGCAGGCTGGAC |
| | | TGCCAGCTAATCAAACAGTAGTTGTCGGATCTAACGTGGAGTTCCACTGCA |
| | | AAGTTTACTCAGACGCCCAACCGCACATTCAATGGCTTAAACACGTGGAGG |
| | | TTAACGGTAGCAAGTATGGACCTGATGGGACTCCCTATGTGACAGTCCTTA |
| | | AGAGTTGGATTTCTAAGAATGCCGAGGCGGATGCGAACTTGAACCTGTTTA |
| | | ACGTAACCGAGCAGGACGAGGGCGAATATCTTTGTCGCGCTAACAACTTCG |
| | | TTGGAATAGCGGAGAAGCCCTTCTGGCTGCATATACGGAAGCCAAAGCCGG |
| | | CGGAGGAGTTGATGGAGATGGATGACTCCGGTTCCGTATACGCCGGAATCC |
| | | TTAGCTACGGAACCGGGCTGGTGCTGTTTATCCTCGTTTTGGTTATTGTTATT |
| | | ATATGCCGCATGAAGATGCCAAACAAAAAGGCAATGAATACTACAACTGTG |
| | | CAGAAAGTGTCAAAATTCCCACTTAAACGGCAAGTCACTGTCTCACTCGAA |
| | | TCTAACTCTTCCATGAACTCCAACACTCCGTTGGTGCGGATTACCAGATTGT |
| | | CCTCATCCGACGGTCCCATGTTGGCAAATGTCAGTGAGCTGGAATTGCCGCC |
| | | CGATCCCAAATGGGAACTTGCAAGATCCCGGCTTACTCTCGGTAAGCCCCT |
| | | GGGCGAAGGTTGCTTTGGACAAGTCGTCATGGCCGAAGCGATTGGAATCGA |
| | | TAAAGACAAACCCAACAAGGCTATAACGGTCGCAGTAAAAATGTTGAAAG |
| | | ATGACGCGACCGACAAGGACCTGTCAGATCTGGTCTCAGAGATGGAAATGA |
| | | TGAAGATGATTGGTAAACACAAGAATATAATCAACTTGCTTGGAGCATGTA |
| | | CGCAAGACGGCCCTCTCTACGTTCTCGTGGAGTATGCCTCTAAGGGCAATTT |
| | | GAGGGAGTACCTGAGAGCTCGCAGACCTCCGGGCATGGATTATAGTTTCGA |
| | | CACTTGTAAGCTGCCGGAGGAGCAACTTACCTTCAAGGATCTTGTAAGTTGC |
| | | GCGTACCAGGTTGCAAGGGGGATGGAATACCTCGCCAGTCAAAAGTGCATA |
| | | CATAGAGATTTGGCAGCGAGGAACGTGCTCGTGACTGAGGACAACGTCATG |
| | | AAAATTGCGGACTTTGGGCTTGCACGGGATGTTCATAACATCGATTACTACA |
| | | AGGAAACTACTAACGGACGGCTCCCTGTGAAGTGGATGGCTCCAGAAGCGC |
| | | TTTTTGATAGAGTTTACACACATCAAAGTGACGTATGGAGTTTTGGAGTATT |
| | | GCTCTGGGAGATTTTCACCTTGGGAGGGTCTCCTTACCCTGGAATACCCGTC |
| | | GAAGAGCTCTTTAAGCTGCTTAAAGAAGGCCACAGGATGGACAAGCCGGCG |
| | | AACTGCACTCATGATCTGTACATGATAATGAGAGAGTGCTGGCACGCCGTT |
| | | CCCTCCCAGCGGCCGACATTCAAGCAGCTGGTGGAAGACCTTGATAGGGTA |
| | | CTCACTATGACGTCCACCGATGAGTATCTGGACTTGTCTGTTCCATTCGAAC |
| | | AATACTCTCCGGCCGGTCAGGATACGCATTCCACTTGCTCCAGTGGTGATGA |
| | | TTCTGTCTTTGCACACGATCTTCTCCCAGACGAACCCTGTCTTCCTAAACAC |
| | | GTACCGTGTAACGGTGTCATAAGGACATAG |
| 99 | FGFR3- | ATGTCAGAAGCTGGTGGCGGCGCCGCTGCCGCAGCCTCACTCCCGAGATCA |
| | N540K-K650E | AGGGCCGGTGGCATGCGCGCGGCATGGGGATCCGTGTGGTGTTTGTGCCTC |
| | | GCGGCGGCTGTCGGAGCTCTGCCGGCTGCAAGGCGCAGGGGAGCGGAACG |
| | | GAGCGGTGGCCAGGCGGCGGAGTATCTCCGGAGTGAAACTGCATTTCTTGA |
| | | GGAGCTTGTTTTCGGTTCAGGGGATACCATCGAGCTTTCCTGTAACACACAA |
| | | TCTTCAAGTGTAAGCGTATTCTGGTTCAAAGATGGTATAGGCATTGCGCCCA |
| | | GTAATAGAACACACATCGGGCAGAAACTTTTGAAGATCATCAACGTTTCAT |
| | | ATGATGATTCCGGACTGTACTCATGTAAGCCAAGGCACAGTAACGAGGTGC |
| | | TTGGGAACTTCACAGTCAGGGTTACAGGCGTACCATTCTGGACGAGGCCCG |
| | | ATAAGATGGAAAAAAAACTTTTGGCAGTGCCCGCAGCCAATACAGTGCGGT |
| | | TCCGCTGCCCCGCAGGTGGGAATCCGACGCCCACAATTTATTGGCTGAAGA |
| | | ATGGCAAGGAGTTCAAAGGAGAACATAGGATCGGCGGAATCAAACTGAGG |
| | | CACCAGCAATGGTCTCTGGTGATGGAATCAGTAGTCCCATCTGACCGCGGT |
| | | AACTACACGTGCGTAGTAGAAAACAAGTATGGGAATATTCGGCATACCTAC |
| | | CAGCTCGATGTGCTCGAGAGATCTCCTCACAGGCCTATCTTGCAGGCTGGAC |
| | | TGCCAGCTAATCAAACAGTAGTTGTCGGATCTAACGTGGAGTTCCACTGCA |
| | | AAGTTTACTCAGACGCCCAACCGCACATTCAATGGCTTAAACACGTGGAGG |
| | | TTAACGGTAGCAAGTATGGACCTGATGGGACTCCCTATGTGACAGTCCTTA |
| | | AGAGTTGGATTTCTAAGAATGCCGAGGCGGATGCGAACTTGAACCTGTTTA |
| | | ACGTAACCGAGCAGGACGAGGGCGAATATCTTTGTCGCGCTAACAACTTCG |
| | | TTGGAATAGCGGAGAAGCCCTTCTGGCTGCATATACGGAAGCCAAAGCCGG |
| | | CGGAGGAGTTGATGGAGATGGATGACTCCGGTTCCGTATACGCCGGAATCC |
| | | TTAGCTACGGAACCGGGCTGGTGCTGTTTATCCTCGTTTTGGTTATTGTTATT |
| | | ATATGCCGCATGAAGATGCCAAACAAAAAGGCAATGAATACTACAACTGTG |
| | | CAGAAAGTGTCAAAATTCCCACTTAAACGGCAAGTCACTGTCTCACTCGAA |
| | | TCTAACTCTTCCATGAACTCCAACACTCCGTTGGTGCGGATTACCAGATTGT |
| | | CCTCATCCGACGGTCCCATGTTGGCAAATGTCAGTGAGCTGGAATTGCCGCC |
| | | CGATCCCAAATGGGAACTTGCAAGATCCCGGCTTACTCTCGGTAAGCCCCT |
| | | GGGCGAAGGTTGCTTTGGACAAGTCGTCATGGCCGAAGCGATTGGAATCGA |
| | | TAAAGACAAACCCAACAAGGCTATAACGGTCGCAGTAAAAATGTTGAAAG |
| | | ATGACGCGACCGACAAGGACCTGTCAGATCTGGTCTCAGAGATGGAAATGA |
| | | TGAAGATGATTGGTAAACACAAGAATATAATCAAGTTGCTTGGAGCATGTA |
| | | CGCAAGACGGCCCTCTCTACGTTCTCGTGGAGTATGCCTCTAAGGGCAATTT |
| | | GAGGGAGTACCTGAGAGCTCGCAGACCTCCGGGCATGGATTATAGTTTCGA |
| | | CACTTGTAAGCTGCCGGAGGAGCAACTTACCTTCAAGGATCTTGTAAGTTGC |
| | | GCGTACCAGGTTGCAAGGGGGATGGAATACCTCGCCAGTCAAAAGTGCATA |
| | | CATAGAGATTTGGCAGCGAGGAACGTGCTCGTGACTGAGGACAACGTCATG |
| | | AAAATTGCGGACTTTGGGCTTGCACGGGATGTTCATAACATCGATTACTACA |
| | | AGGAAACTACTAACGGACGGCTCCCTGTGAAGTGGATGGCTCCAGAAGCGC |
| | | TTTTTGATAGAGTTTACACACATCAAAGTGACGTATGGAGTTTTGGAGTATT |
| | | GCTCTGGGAGATTTTCACCTTGGGAGGGTCTCCTTACCCTGGAATACCCGTC |
| | | GAAGAGCTCTTTAAGCTGCTTAAAGAAGGCCACAGGATGGACAAGCCGGCG |
| | | AACTGCACTCATGATCTGTACATGATAATGAGAGAGTGCTGGCACGCCGTT |
| | | CCCTCCCAGCGGCCGACATTCAAGCAGCTGGTGGAAGACCTTGATAGGGTA |
| | | CTCACTATGACGTCCACCGATGAGTATCTGGACTTGTCTGTTCCATTCGAAC |
| | | AATACTCTCCGGCCGGTCAGGATACGCATTCCACTTGCTCCAGTGGTGATGA |
| | | TTCTGTCTTTGCACACGATCTTCTCCCAGACGAACCCTGTCTTCCTAAACAC |
| | | GTACCGTGTAACGGTGTCATAAGGACATAG |
| 100 | myrist- | ATGGGATCATCCAAGTCAAAACCGAAAGACCCGTCACAGAGACGCATGAA |
| | FGFR3- | GATGCCAAACAAAAAGGCAATGAATACTACAACTGTGCAGAAAGTGTCAA |
| | K650E | AATTCCCACTTAAACGGCAAGTCACTGTCTCACTCGAATCTAACTCTTCCAT |
| | | GAACTCCAACACTCCGTTGGTGCGGATTACCAGATTGTCCTCATCCGACGGT |
| | | CCCATGTTGGCAAATGTCAGTGAGCTGGAATTGCCGCCCGATCCCAAATGG |
| | | GAACTTGCAAGATCCCGGCTTACTCTCGGTAAGCCCCTGGGCGAAGGTTGC |
| | | TTTGGACAAGTCGTCATGGCCGAAGCGATTGGAATCGATAAAGACAAACCC |
| | | AACAAGGCTATAACGGTCGCAGTAAAAATGTTGAAAGATGACGCGACCGA |
| | | CAAGGACCTGTCAGATCTGGTCTCAGAGATGGAAATGATGAAGATGATTGG |
| | | TAAACACAAGAATATAATCAACTTGCTTGGAGCATGTACGCAAGACGGCCC |
| | | TCTCTACGTTCTCGTGGAGTATGCCTCTAAGGGCAATTTGAGGGAGTACCTG |
| | | AGAGCTCGCAGACCTCCGGGCATGGATTATAGTTTCGACACTTGTAAGCTG |
| | | CCGGAGGAGCAACTTACCTTCAAGGATCTTGTAAGTTGCGCGTACCAGGTT |
| | | GCAAGGGGGATGGAATACCTCGCCAGTCAAAAGTGCATACATAGAGATTTG |
| | | GCAGCGAGGAACGTGCTCGTGACTGAGGACAACGTCATGAAAATTGCGGAC |
| | | TTTGGGCTTGCACGGGATGTTCATAACATCGATTACTACAAGGAAACTACTA |
| | | ACGGACGGCTCCCTGTGAAGTGGATGGCTCCAGAAGCGCTTTTTGATAGAG |
| | | TTTACACACATCAAAGTGACGTATGGAGTTTTGGAGTATTGCTCTGGGAGAT |
| | | TTTCACCTTGGGAGGGTCTCCTTACCCTGGAATACCCGTCGAAGAGCTCTTT |
| | | AAGCTGCTTAAAGAAGGCCACAGGATGGACAAGCCGGCGAACTGCACTCAT |
| | | GATCTGTACATGATAATGAGAGAGTGCTGGCACGCCGTTCCCTCCCAGCGG |
| | | CCGACATTCAAGCAGCTGGTGGAAGACCTTGATAGGGTACTCACTATGACG |
| | | TCCACCGATGAGTATCTGGACTTGTCTGTTCCATTCGAACAATACTCTCCGG |
| | | CCGGTCAGGATACGCATTCCACTTGCTCCAGTGGTGATGATTCTGTCTTTGC |
| | | ACACGATCTTCTCCCAGACGAACCCTGTCTTCCTAAACACGTACCGTGTAAC |
| | | GGTGTCATAAGGACATAG |
| 101 | FGFR4-WT | ATGCTTCCTCTGCGCCTGGTTCTCGCTGGCCTCTTGGTCGCAGCGGGTTCAG |
| | | CGGCGAGTCATAGGGGAGAAATGGAGCCGGAACTCTTTGAGTCTCCACTCT |
| | | TGGAATCCGAAGAAGAACACCTCCTTCTGGACCCAGGAAACGCATTGAAAC |
| | | TCTATTGTGACGTAAACCAGTCCGGAGCTAGTGTGGTTTGGTATAAGGAGA |
| | | GTAGACCTCTGCTGCCAGGGCCCCGCGTCAGATTGCAACAAAGCGTTCTTG |
| | | AAATAGCGGAAGTAGCTTACGAGGATTCCGGCCTCTACGTCTGTAGAGCTC |
| | | GCGGAACCGGTGAGGTCCTTAGGAACTTCACCATATCAGTTGTAGATTCACT |
| | | TGCCTCAGGCGATGACGATGAAGACAGCGATGGGGATGGTCCACATGGAG |
| | | ACCGCTCTGAAGAACCAGTATACGTTCACAGAGCACCTTATTGGACCCATC |
| | | CACACAGGATGGATAAAAAACTCTACGCTGTTCCTGCGGGCAATACCGTGA |
| | | AATTCCGCTGTCCAGCGAGTGGGTCTCCAAGCCCGTCCATTAGATGGTTTAA |
| | | GAATGGCAGAGAGTTTCGCGGGGAGCACAGAATAGGGGGCATTAGGCTCC |
| | | GGCATCAGCATTGGTCACTCGTTATGGAGTCAGTCGTGCCGTCTGATAGGG |
| | | GGAATTACACCTGCTTGGTAGAGAACCGGTTTGGTTCAATCCGCTATAGTTA |
| | | TCTGCTGGATGTCCTCGAACGCTCCCCACACAGACCCATCTTGCAAGCTGGA |
| | | CTTCCAGCTAACACAACAGCTCTGGTAGGTTCAGATGTGGAATTTTTCTGCA |
| | | AGGTATACTCTGATGCTCAACCGCACATACAGTGGTTGAAACACATTGAAG |
| | | TTAACGGGTCCTCATATGGGCCAGACGGTGTACCCTACGTGCAGGTACTGA |
| | | AGACGGCCGACATTAATTCATCTGAGGTTGAGGTGCTGTATTTGCGGAACG |
| | | TCACAATGGAAGACGCCGGGGAGTATACTTGTCTTGCCGGTAATAGTATTG |
| | | GGCTGTCCTATCAGTCCGCGTGGCTCACCGTCCTGCCAGAAGAGCTGGTTCA |
| | | TGAGGCTGAGGCACCTGAGGCGAAATACACCGACATCATAATTTACACTTC |
| | | CGGATCATTGGCCGTGGCAATGGCTCTTATCATCGTCGTTCTGTGTAGGATG |
| | | CAAACTCAGAGCTCTAAACAACCCCTTGAACCCATGGCAGTACACAAATTG |
| | | AGCAAATTTCCTCTTATTAGACAGTTTTCCCTTGACTCAAGTAGCTCAGGGA |
| | | AATCTAGCACATCACTTATGCGGGTGACGAGACTGTCTTCCAGCTGCGCGCC |
| | | CATGTTGGCTGGGGTGGTGGAACTTGATCTGCCTCTGGACAGCAAGTGGGA |
| | | ATTTCCGAGGGAGAAACTGGTTCTTGGGAAGCCGCTCGGCGAAGGCTGTTT |
| | | TGGTCAGGTGGTCAGGGCGGAAGCGTACGGGATCGACAGACAGTGGCCTG |
| | | ATCGCGCAGTTACTGTCGCAGTAAAAATGCTGAAAGACAACGCTACTGATA |
| | | AGGATCTGGCAGACCTGATAAGTGAAATGGAGATGATGAAACTGATGGAC |
| | | AAACACAAGAACATCATTAATCTCTTGGGTGTATGTACACAAGATGGGCCT |
| | | CTGTATGTTATAGTAGAGTTTGCGGCCAAAGGCAACCTTCGGGAGTATCTTC |
| | | GCGCTAGAAGACCGCCAATGCCCGACTACACGTTCGATATTACAGAACTCC |
| | | ATGAGGAACAACTTTGTTTTAAGGATCTTGTTAGCTGTGTGTATCAAGTCGC |
| | | CCGGGGGATGGAGTATCTGGAATCAAGACGGTGTATACACCGCGACCTCGC |
| | | TGCCAGAAACGTTCTCGTCACGGCGGAAAATGTGATGAAGATCGCCGACTT |
| | | CGGACTTGCCAGGGATGTCCATGATATAGACTATTACAAAAAAACATCTAA |
| | | TGGGCGGCTCCCTGTCAAGTGGATGGCGCCCGAAGCACTGTTTGACAGAGT |
| | | ATACACGCACCAGTCTGACGTGTGGTCATTTGGCATACTGATGTGGGAAATT |
| | | TTTACACTCGGTGGTTCACCTTATCCTGGCATCCCTGTTGAGGAGCTTTTTAA |
| | | ATTGCTCAAAGAGGGCCACAGAATGGACTGTCCTAGTAACTGCACCCATGA |
| | | GCTGTATATGCTCATGCGCGAGTGCTGGCATGCGGTGCCTAGTCAAAGGCC |
| | | AACCTTCAAACAGCTCGTCGAAGGCCTGGACAAGATTCTTGCTGCAATAAG |
| | | CGAGGAGTACCTCGACTTGTCTATGCCATTCGAGCAATACTCACCTTCTTGT |
| | | GAAGACACGACGAGTACATGCAGCAGCGACGACTCTGTATTTACACACGAC |
| | | CCTTTGCCCCTTGCTCCTTGCCTGTTTGCCTGTCCTAGTGGCCGCACCTAG |
| 102 | FGFR4- | ATGCTTCCTCTGCGCCTGGTTCTCGCTGGCCTCTTGGTCGCAGCGGGTTCAG |
| | Y367C | CGGCGAGTCATAGGGGAGAAATGGAGCCGGAACTCTTTGAGTCTCCACTCT |
| | | TGGAATCCGAAGAAGAACACCTCCTTCTGGACCCAGGAAACGCATTGAAAC |
| | | TCTATTGTGACGTAAACCAGTCCGGAGCTAGTGTGGTTTGGTATAAGGAGA |
| | | GTAGACCTCTGCTGCCAGGGCCCCGCGTCAGATTGCAACAAAGCGTTCTTG |
| | | AAATAGCGGAAGTAGCTTACGAGGATTCCGGCCTCTACGTCTGTAGAGCTC |
| | | GCGGAACCGGTGAGGTCCTTAGGAACTTCACCATATCAGTTGTAGATTCACT |
| | | TGCCTCAGGCGATGACGATGAAGACAGCGATGGGGATGGTCCACATGGAG |
| | | ACCGCTCTGAAGAACCAGTATACGTTCACAGAGCACCTTATTGGACCCATC |
| | | CACACAGGATGGATAAAAAACTCTACGCTGTTCCTGCGGGCAATACCGTGA |
| | | AATTCCGCTGTCCAGCGAGTGGGTCTCCAAGCCCGTCCATTAGATGGTTTAA |
| | | GAATGGCAGAGAGTTTCGCGGGGAGCACAGAATAGGGGGCATTAGGCTCC |
| | | GGCATCAGCATTGGTCACTCGTTATGGAGTCAGTCGTGCCGTCTGATAGGG |
| | | GGAATTACACCTGCTTGGTAGAGAACCGGTTTGGTTCAATCCGCTATAGTTA |
| | | TCTGCTGGATGTCCTCGAACGCTCCCCACACAGACCCATCTTGCAAGCTGGA |
| | | CTTCCAGCTAACACAACAGCTCTGGTAGGTTCAGATGTGGAATTTTTCTGCA |
| | | AGGTATACTCTGATGCTCAACCGCACATACAGTGGTTGAAACACATTGAAG |
| | | TTAACGGGTCCTCATATGGGCCAGACGGTGTACCCTACGTGCAGGTACTGA |
| | | AGACGGCCGACATTAATTCATCTGAGGTTGAGGTGCTGTATTTGCGGAACG |
| | | TCACAATGGAAGACGCCGGGGAGTATACTTGTCTTGCCGGTAATAGTATTG |
| | | GGCTGTCCTATCAGTCCGCGTGGCTCACCGTCCTGCCAGAAGAGCTGGTTCA |
| | | TGAGGCTGAGGCACCTGAGGCGAAATGTACCGACATCATAATTTACACTTC |
| | | CGGATCATTGGCCGTGGCAATGGCTCTTATCATCGTCGTTCTGTGTAGGATG |
| | | CAAACTCAGAGCTCTAAACAACCCCTTGAACCCATGGCAGTACACAAATTG |
| | | AGCAAATTTCCTCTTATTAGACAGTTTTCCCTTGACTCAAGTAGCTCAGGGA |
| | | AATCTAGCACATCACTTATGCGGGTGACGAGACTGTCTTCCAGCTGCGCGCC |
| | | CATGTTGGCTGGGGTGGTGGAACTTGATCTGCCTCTGGACAGCAAGTGGGA |
| | | ATTTCCGAGGGAGAAACTGGTTCTTGGGAAGCCGCTCGGCGAAGGCTGTTT |
| | | TGGTCAGGTGGTCAGGGCGGAAGCGTACGGGATCGACAGACAGTGGCCTG |
| | | ATCGCGCAGTTACTGTCGCAGTAAAAATGCTGAAAGACAACGCTACTGATA |
| | | AGGATCTGGCAGACCTGATAAGTGAAATGGAGATGATGAAACTGATGGAC |
| | | AAACACAAGAACATCATTAATCTCTTGGGTGTATGTACACAAGATGGGCCT |
| | | CTGTATGTTATAGTAGAGTTTGCGGCCAAAGGCAACCTTCGGGAGTATCTTC |
| | | GCGCTAGAAGACCGCCAATGCCCGACTACACGTTCGATATTACAGAACTCC |
| | | ATGAGGAACAACTTTGTTTTAAGGATCTTGTTAGCTGTGTGTATCAAGTCGC |
| | | CCGGGGGATGGAGTATCTGGAATCAAGACGGTGTATACACCGCGACCTCGC |
| | | TGCCAGAAACGTTCTCGTCACGGCGGAAAATGTGATGAAGATCGCCGACTT |
| | | CGGACTTGCCAGGGATGTCCATGATATAGACTATTACAAAAAAACATCTAA |
| | | TGGGCGGCTCCCTGTCAAGTGGATGGCGCCCGAAGCACTGTTTGACAGAGT |
| | | ATACACGCACCAGTCTGACGTGTGGTCATTTGGCATACTGATGTGGGAAATT |
| | | TTTACACTCGGTGGTTCACCTTATCCTGGCATCCCTGTTGAGGAGCTTTTTAA |
| | | ATTGCTCAAAGAGGGCCACAGAATGGACTGTCCTAGTAACTGCACCCATGA |
| | | GCTGTATATGCTCATGCGCGAGTGCTGGCATGCGGTGCCTAGTCAAAGGCC |
| | | AACCTTCAAACAGCTCGTCGAAGGCCTGGACAAGATTCTTGCTGCAATAAG |
| | | CGAGGAGTACCTCGACTTGTCTATGCCATTCGAGCAATACTCACCTTCTTGT |
| | | GAAGACACGACGAGTACATGCAGCAGCGACGACTCTGTATTTACACACGAC |
| | | CCTTTGCCCCTTGCTCCTTGCCTGTTTGCCTGTCCTAGTGGCCGCACCTAG |
| 103 | FGFR4- | ATGCTTCCTCTGCGCCTGGTTCTCGCTGGCCTCTTGGTCGCAGCGGGTTCAG |
| | K645E | CGGCGAGTCATAGGGGAGAAATGGAGCCGGAACTCTTTGAGTCTCCACTCT |
| | | TGGAATCCGAAGAAGAACACCTCCTTCTGGACCCAGGAAACGCATTGAAAC |
| | | TCTATTGTGACGTAAACCAGTCCGGAGCTAGTGTGGTTTGGTATAAGGAGA |
| | | GTAGACCTCTGCTGCCAGGGCCCCGCGTCAGATTGCAACAAAGCGTTCTTG |
| | | AAATAGCGGAAGTAGCTTACGAGGATTCCGGCCTCTACGTCTGTAGAGCTC |
| | | GCGGAACCGGTGAGGTCCTTAGGAACTTCACCATATCAGTTGTAGATTCACT |
| | | TGCCTCAGGCGATGACGATGAAGACAGCGATGGGGATGGTCCACATGGAG |
| | | ACCGCTCTGAAGAACCAGTATACGTTCACAGAGCACCTTATTGGACCCATC |
| | | CACACAGGATGGATAAAAAACTCTACGCTGTTCCTGCGGGCAATACCGTGA |
| | | AATTCCGCTGTCCAGCGAGTGGGTCTCCAAGCCCGTCCATTAGATGGTTTAA |
| | | GAATGGCAGAGAGTTTCGCGGGGAGCACAGAATAGGGGGCATTAGGCTCC |
| | | GGCATCAGCATTGGTCACTCGTTATGGAGTCAGTCGTGCCGTCTGATAGGG |
| | | GGAATTACACCTGCTTGGTAGAGAACCGGTTTGGTTCAATCCGCTATAGTTA |
| | | TCTGCTGGATGTCCTCGAACGCTCCCCACACAGACCCATCTTGCAAGCTGGA |
| | | CTTCCAGCTAACACAACAGCTCTGGTAGGTTCAGATGTGGAATTTTTCTGCA |
| | | AGGTATACTCTGATGCTCAACCGCACATACAGTGGTTGAAACACATTGAAG |
| | | TTAACGGGTCCTCATATGGGCCAGACGGTGTACCCTACGTGCAGGTACTGA |
| | | AGACGGCCGACATTAATTCATCTGAGGTTGAGGTGCTGTATTTGCGGAACG |
| | | TCACAATGGAAGACGCCGGGGAGTATACTTGTCTTGCCGGTAATAGTATTG |
| | | GGCTGTCCTATCAGTCCGCGTGGCTCACCGTCCTGCCAGAAGAGCTGGTTCA |
| | | TGAGGCTGAGGCACCTGAGGCGAAATACACCGACATCATAATTTACACTTC |
| | | CGGATCATTGGCCGTGGCAATGGCTCTTATCATCGTCGTTCTGTGTAGGATG |
| | | CAAACTCAGAGCTCTAAACAACCCCTTGAACCCATGGCAGTACACAAATTG |
| | | AGCAAATTTCCTCTTATTAGACAGTTTTCCCTTGACTCAAGTAGCTCAGGGA |
| | | AATCTAGCACATCACTTATGCGGGTGACGAGACTGTCTTCCAGCTGCGCGCC |
| | | CATGTTGGCTGGGGTGGTGGAACTTGATCTGCCTCTGGACAGCAAGTGGGA |
| | | ATTTCCGAGGGAGAAACTGGTTCTTGGGAAGCCGCTCGGCGAAGGCTGTTT |
| | | TGGTCAGGTGGTCAGGGCGGAAGCGTACGGGATCGACAGACAGTGGCCTG |
| | | ATCGCGCAGTTACTGTCGCAGTAAAAATGCTGAAAGACAACGCTACTGATA |
| | | AGGATCTGGCAGACCTGATAAGTGAAATGGAGATGATGAAACTGATGGAC |
| | | AAACACAAGAACATCATTAATCTCTTGGGTGTATGTACACAAGATGGGCCT |
| | | CTGTATGTTATAGTAGAGTTTGCGGCCAAAGGCAACCTTCGGGAGTATCTTC |
| | | GCGCTAGAAGACCGCCAATGCCCGACTACACGTTCGATATTACAGAACTCC |
| | | ATGAGGAACAACTTTGTTTTAAGGATCTTGTTAGCTGTGTGTATCAAGTCGC |
| | | CCGGGGGATGGAGTATCTGGAATCAAGACGGTGTATACACCGCGACCTCGC |
| | | TGCCAGAAACGTTCTCGTCACGGCGGAAAATGTGATGAAGATCGCCGACTT |
| | | CGGACTTGCCAGGGATGTCCATGATATAGACTATTACAAAGAAACATCTAA |
| | | TGGGCGGCTCCCTGTCAAGTGGATGGCGCCCGAAGCACTGTTTGACAGAGT |
| | | ATACACGCACCAGTCTGACGTGTGGTCATTTGGCATACTGATGTGGGAAATT |
| | | TTTACACTCGGTGGTTCACCTTATCCTGGCATCCCTGTTGAGGAGCTTTTTAA |
| | | ATTGCTCAAAGAGGGCCACAGAATGGACTGTCCTAGTAACTGCACCCATGA |
| | | GCTGTATATGCTCATGCGCGAGTGCTGGCATGCGGTGCCTAGTCAAAGGCC |
| | | AACCTTCAAACAGCTCGTCGAAGGCCTGGACAAGATTCTTGCTGCAATAAG |
| | | CGAGGAGTACCTCGACTTGTCTATGCCATTCGAGCAATACTCACCTTCTTGT |
| | | GAAGACACGACGAGTACATGCAGCAGCGACGACTCTGTATTTACACACGAC |
| | | CCTTTGCCCCTTGCTCCTTGCCTGTTTGCCTGTCCTAGTGGCCGCACCTAG |
| 104 | FGFR4- | ATGCTTCCTCTGCGCCTGGTTCTCGCTGGCCTCTTGGTCGCAGCGGGTTCAG |
| | Y367C-K645E | CGGCGAGTCATAGGGGAGAAATGGAGCCGGAACTCTTTGAGTCTCCACTCT |
| | | TGGAATCCGAAGAAGAACACCTCCTTCTGGACCCAGGAAACGCATTGAAAC |
| | | TCTATTGTGACGTAAACCAGTCCGGAGCTAGTGTGGTTTGGTATAAGGAGA |
| | | GTAGACCTCTGCTGCCAGGGCCCCGCGTCAGATTGCAACAAAGCGTTCTTG |
| | | AAATAGCGGAAGTAGCTTACGAGGATTCCGGCCTCTACGTCTGTAGAGCTC |
| | | GCGGAACCGGTGAGGTCCTTAGGAACTTCACCATATCAGTTGTAGATTCACT |
| | | TGCCTCAGGCGATGACGATGAAGACAGCGATGGGGATGGTCCACATGGAG |
| | | ACCGCTCTGAAGAACCAGTATACGTTCACAGAGCACCTTATTGGACCCATC |
| | | CACACAGGATGGATAAAAAACTCTACGCTGTTCCTGCGGGCAATACCGTGA |
| | | AATTCCGCTGTCCAGCGAGTGGGTCTCCAAGCCCGTCCATTAGATGGTTTAA |
| | | GAATGGCAGAGAGTTTCGCGGGGAGCACAGAATAGGGGGCATTAGGCTCC |
| | | GGCATCAGCATTGGTCACTCGTTATGGAGTCAGTCGTGCCGTCTGATAGGG |
| | | GGAATTACACCTGCTTGGTAGAGAACCGGTTTGGTTCAATCCGCTATAGTTA |
| | | TCTGCTGGATGTCCTCGAACGCTCCCCACACAGACCCATCTTGCAAGCTGGA |
| | | CTTCCAGCTAACACAACAGCTCTGGTAGGTTCAGATGTGGAATTTTTCTGCA |
| | | AGGTATACTCTGATGCTCAACCGCACATACAGTGGTTGAAACACATTGAAG |
| | | TTAACGGGTCCTCATATGGGCCAGACGGTGTACCCTACGTGCAGGTACTGA |
| | | AGACGGCCGACATTAATTCATCTGAGGTTGAGGTGCTGTATTTGCGGAACG |
| | | TCACAATGGAAGACGCCGGGGAGTATACTTGTCTTGCCGGTAATAGTATTG |
| | | GGCTGTCCTATCAGTCCGCGTGGCTCACCGTCCTGCCAGAAGAGCTGGTTCA |
| | | TGAGGCTGAGGCACCTGAGGCGAAATGTACCGACATCATAATTTACACTTC |
| | | CGGATCATTGGCCGTGGCAATGGCTCTTATCATCGTCGTTCTGTGTAGGATG |
| | | CAAACTCAGAGCTCTAAACAACCCCTTGAACCCATGGCAGTACACAAATTG |
| | | AGCAAATTTCCTCTTATTAGACAGTTTTCCCTTGACTCAAGTAGCTCAGGGA |
| | | AATCTAGCACATCACTTATGCGGGTGACGAGACTGTCTTCCAGCTGCGCGCC |
| | | CATGTTGGCTGGGGTGGTGGAACTTGATCTGCCTCTGGACAGCAAGTGGGA |
| | | ATTTCCGAGGGAGAAACTGGTTCTTGGGAAGCCGCTCGGCGAAGGCTGTTT |
| | | TGGTCAGGTGGTCAGGGCGGAAGCGTACGGGATCGACAGACAGTGGCCTG |
| | | ATCGCGCAGTTACTGTCGCAGTAAAAATGCTGAAAGACAACGCTACTGATA |
| | | AGGATCTGGCAGACCTGATAAGTGAAATGGAGATGATGAAACTGATGGAC |
| | | AAACACAAGAACATCATTAATCTCTTGGGTGTATGTACACAAGATGGGCCT |
| | | CTGTATGTTATAGAAGAGTTTGCGGCCAAAGGCAACCTTCGGGAGTATCTTC |
| | | GCGCTAGAAGACCGCCAATGCCCGACTACACGTTCGATATTACAGAACTCC |
| | | ATGAGGAACAACTTTGTTTTAAGGATCTTGTTAGCTGTGTGTATCAAGTCGC |
| | | CCGGGGGATGGAGTATCTGGAATCAAGACGGTGTATACACCGCGACCTCGC |
| | | TGCCAGAAACGTTCTCGTCACGGCGGAAAATGTGATGAAGATCGCCGACTT |
| | | CGGACTTGCCAGGGATGTCCATGATATAGACTATTACAAAAAAACATCTAA |
| | | TGGGCGGCTCCCTGTCAAGTGGATGGCGCCCGAAGCACTGTTTGACAGAGT |
| | | ATACACGCACCAGTCTGACGTGTGGTCATTTGGCATACTGATGTGGGAAATT |
| | | TTTACACTCGGTGGTTCACCTTATCCTGGCATCCCTGTTGAGGAGCTTTTTAA |
| | | ATTGCTCAAAGAGGGCCACAGAATGGACTGTCCTAGTAACTGCACCCATGA |
| | | GCTGTATATGCTCATGCGCGAGTGCTGGCATGCGGTGCCTAGTCAAAGGCC |
| | | AACCTTCAAACAGCTCGTCGAAGGCCTGGACAAGATTCTTGCTGCAATAAG |
| | | CGAGGAGTACCTCGACTTGTCTATGCCATTCGAGCAATACTCACCTTCTTGT |
| | | GAAGACACGACGAGTACATGCAGCAGCGACGACTCTGTATTTACACACGAC |
| | | CCTTTGCCCCTTGCTCCTTGCCTGTTTGCCTGTCCTAGTGGCCGCACCTAG |
| 105 | IGF1(WT) | ATGGAAAAAATCAACAGTCTTTCAACACAATTAGTTAAGTGCTGCTTTTGTG |
| | | ATTTCTTGAAGGTGAAGATGCACACTGTGTCCTACATTCATTTCTTCTACCTT |
| | | GGCCTGTGTTTGCTTACCTTAACCAGTTCTGCTGCTGCCGGCCCAGAAACAC |
| | | TGTGTGGTGCTGAGCTGGTTGATGCTCTTCAGTTCGTATGTGGAGACAGAGG |
| | | CTTCTACTTCAGTAAGCCTACAGGGTATGGATCCAGCAGTAGACGCTTACAC |
| | | CACAAGGGAATAGTGGATGAGTGCTGCTTCCAGAGTTGTGACCTGAGGAGG |
| | | CTGGAGATGTACTGTGCTCCAATAAAGCCACCTAAATCTGCACGCTCTGTAC |
| | | GTGCTCAGCGCCACACTGATATGCCAAAAGCACAAAAGGAAGTGCATTTGA |
| | | AGAATACAAGTAGAGGGAACACAGGAAACAGAAACTACAGAATGTAA |
| 106 | IGF1 Trunc | GGCCCAGAAACACTGTGTGGTGCTGAGCTGGTTGATGCTCTTCAGTTCGTAT |
| | (truncated) | GTGGAGACAGAGGCTTCTACTTCAGTAAGCCTACAGGGTATGGATCCAGCA |
| | | GTAGACGCTTACACCACAAGGGAATAGTGGATGAGTGCTGCTTCCAGAGTT |
| | | GTGACCTGAGGAGGCTGGAGATGTACTGTGCTCCAATAAAGCCACCTAAAT |
| | | CTGCATAA |
| 107 | IGF1R | ATGAAATCTGGGGCTGGGGGAGGGACCCTCGCCGTATTCTGTGGGCTTTTGT |
| | | TGGCGTTCGCCGCACTCTGTCTCTGTCCGACCAATGGTGAAATATGCGGCCC |
| | | GAATGTGGATATTAGAAACGATATTCACGAGCTCAAGAGGCTCGAAAATTG |
| | | TACTGTAGTGGAGGGGTTTCTGCAAATACTGCTTATCAGTAAGGCGGAAGA |
| | | TTACCGCAACTTCCGCTTTCCTAAGCTGACTGTGATCACAGACTATCTCCTG |
| | | CTGTTCAGAGTTGCCGGACTTGAATCCCTCTCAGATCTGTTTCCTAACCTCA |
| | | CAGTGATCAGGGGTAGAAACCTGTTCTACAACTATGCTCTCGTAATATTTGA |
| | | GATGACGAACCTTAAGGAGATTGGACTCCACAATCTTCGCAACATAACCAG |
| | | GGGTGCTATAAGGATCGAAAAAAATAGCGACCTGTGCTATCTTTCTACAGT |
| | | AGACTGGAGTCTCATATTGGACGCAGTATCCAACAACTATATAGTTGGCAA |
| | | CAAGCCTCCCAAAGAATGCGGTGATCTCTGTCCTGGAACTATGGAGGAAAA |
| | | ACCTCTCTGCGAAAAGACTAGTATAAACAACGAGTACAACTATAGGTGTTG |
| | | GACTACCAACCACTGCCAGAAGATGTGCCCGAGCAGCTGCGGCAAGCGCGC |
| | | GTGCACCGATCAGAATGAATGCTGCCACCCCGAATGTCTTGGTAGCTGTAC |
| | | GGCTCCCGACAATAACACAGCCTGCGTTGCGTGTCGCAATTACTATTATGAG |
| | | GGTGTCTGTATGCCTACCTGTCCGCCTAACACATATAAGTTCGAGGGGTGGC |
| | | GCTGCGTGACAAAAGAATTTTGTTCCAAGGTCCCAGCAACGGAGACGTCTG |
| | | ACTATGAAAGGTTTGTAATTCATAATGATGAATGTATGGCGGAATGCCCAT |
| | | CTGGATTTATCAGGAACGGTAGTCAGAGCATGTTTTGCTCCCCATGTGAAGG |
| | | CCCATGTCCCAAAATTTGTGAAGACGGGAAGACGAAGACAATAGATAGCGT |
| | | CACTTCTGCTCAAATGCTTCAGGGATGTACCATCTTGAAAGGAAATCTCTTG |
| | | ATTAACATTCGCCGGGGTAATAATATAGCAAGTGAGCTTGAAAACTTTATG |
| | | GGGCTCATAGAAACGGTAACTGGGTATGTCAAGATCCGCCATAGTCATGCA |
| | | CTCGTGTCACTTTCATTCTTGAAGAATCTCCGCTATATACTCGGCGAAGAGC |
| | | AAGTTGACGGCAACTACTCATTTTACGTTCTCGATAATCATAATTTGCAGCA |
| | | GCTTTGGGACTGGAATCACCACAACTTGACCATTAAAGAAGGAAAGATGTA |
| | | CTTCGCTTTCAATCCGAAACTTTGTGTATCCGAAATTTACCGCATGGAAGAG |
| | | GTGTCTGGAACTAAAGGACGCCAGTCAAAAGGAGATATAAATCCCAGGAAT |
| | | AATGGAGAAAGGGCGTCCTGTGAGAGCCATATATTGAGATTCGTGAGCAAT |
| | | ACCACGCTGAAGAACCGGATAAAACTCACCTGGGAGAGATACAGGCCCCC |
| | | GGATTACCGGGACCTCATCTCTTTCACGGTTTATTACAAGGAGGCCCCCTTC |
| | | AAAAACGTCACAGAGTACGATGGGCAAGATGCCTGCGGCTCCAACTCTTGG |
| | | AACATGGTCGACGTAGACTTGCCCCCTAACAAAGAGAATGACCCTGGTATA |
| | | TTGCTTCAAGGACTTAAACCTTGGACGCAGTACGCCATATACGTCAAAGCC |
| | | GTGACCCTTACAATGATGGAAAACCACCACATCCACGGGGCTAAATCCGAG |
| | | ATTGTTTATATAAGGACAAATGCAGCCGTCCCCAGCATACCTCTCGACGTAA |
| | | TATCTGCCTCTAATAGCAGCAGCCAGCTCATTGTGAAGTGGAATCCTCCTTC |
| | | ACTTCCCAACGGGAATCTGTCTTATTACATTGTCCGCTGGCAACAACAACCT |
| | | CAGGATTCTTATTTGTATCGGCATAACTACTGTAGTAAAGACAAGGTGCCG |
| | | ATTCGCCGGTACGCGGACGGTACTATTGACACGGAAGAAGCAACAGAGCCA |
| | | ACAAAACCCGAAGGATGCGGCGGTGAAAAAGGTCCGTGTTGTGCGTGCCCT |
| | | AAGACCGAAGCTGAGAAGCAAGCTGAAAAGGAAGAAGCTGAATACCGCAA |
| | | GGTGTTTGAGAACTTCCTGCATAACTCCATCTTCGTTCCTCGCCCGGATAGA |
| | | AAGAGGCGCGACGTGTTCAGGATTGCTAACGCCACGCTCGCTACGAGGAAT |
| | | CGGAACATTACTGGTGCGGATCACTTCACCAATGCATCCGATGCGGAAGAG |
| | | TCCGAAGTAGAGTACCCATTTTTTGAGACGAAAGTCGACGGGAAGGAGAGA |
| | | ACGGTAATATCTCATTTGCAGCCTTTTACCCTTTATAGAATAGATATCCACA |
| | | GCTGCAATCACGAAGCAGACACCCTTGGTTGTAGTGCTTCTAACTTCGTGTT |
| | | TGCCCGCACGATGCCCTCTGAAGGCGCTGACAATATACCCGGAACCGTAGC |
| | | ATGGGAAGCGAAGGAGGAGAACACGGTTTATCTTAAATGGTTGGAACCCAC |
| | | CAACCCTAACGGACTTATCTTGATGTACGAGATTAAATATGGACAACACGG |
| | | GGAGGAAAAGAGGGAGTGTGTCAGTAGGCAAGAATACAAAAAACTTGGTG |
| | | GCGCCAAATTGACACACTTGAACCCTGGTAATTACAGCGCGCGCGTGCAGG |
| | | CGACTAGCTTGGCAGGTAATGGTTCTTGGACTGAACCCGTCTCCTTCTATGT |
| | | TCAGCCCAAATCCGCAAATTACGACAACTTTCTCCATTTGATAATAGTCCTT |
| | | CCAATAGCCTTCCTGTTGATAATAGGTGGCCTCCTCATTATGTTGTACGTCTT |
| | | TAATAAGAAACGGAACTCTGACCGGCTCGGAAATGGTGTTCTGTATGCAAG |
| | | TGTTAATCCAGAATATTTCTCTGCATCAGACGTTTATGTGCCGGACGAGTGG |
| | | GAAGTGCCAAGGGAGAAAATTACAATGTGCAGAGAACTCGGACAGGGTTC |
| | | TTTTGGCATGGTTTACGAGGGCATCGCAAAAGGCGTGGTGAAGGACGAACC |
| | | CGAGACGAGAGTTGCTATCAAAACAGTCAATGAGTCAGCTTCTATGCGCGA |
| | | ACGGATTGAGTTTTTGAATGAGGCCAGTGTAATGAAGGAGTTTAACTGCCA |
| | | CCACGTCGTACGGTTGCTTGGCGTCGTGTCTCAAGGACAGCCCACCCTCGTT |
| | | ATTATGGAGTTGATGACCCGGGGGGATCTCAAGTCCTACTTGAGGTCTCTTC |
| | | GCCCTGACACTGAGTCAAACCCAGGGCAGGCACCTCCTACGCTGAAAAAAA |
| | | TGATACAAATGGCTGGAGAAATCGCCGATGGGATGGCGTATCTCAACGCTA |
| | | ATAAGTTTGTACACCGCGATCTGGCCGCAAGAAATTGTATGGTCGCAGAGG |
| | | ATTTTACTGTCAAGATCGGCGATTTCGGCATGACACGCGACATCTATGAGAC |
| | | CGACTACTATAGGAAGGGGGGGAAGGGTTTGCTCCCAGTAAGGTGGATGTC |
| | | ACCGGAGAGCCTCAAGGATGGGGTGTTCACGACTCACAGCGATGTCTGGTC |
| | | TTTTGGAGTTGTACTCTGGGAAATAGCTACACTGGCAGAGCAACCCTACCA |
| | | AGGAATGACAAATGAACAAGTCCTCCGCTTTGTAATGGAAGGGGGTCTTCT |
| | | CGAAAAACCGGACAACTGCCCAGACATGCTTTTCGAGCTGATGCGCATGTG |
| | | CTGGCAGTACAATCCCAAAATGCGCCCGTCTTTTCTTGAAATAATCTCAAGC |
| | | ATCAAGGATGAACTTGACCCGGCCTTTAAAGAAGTATCATTTTTCTATTCCG |
| | | AAGAAAATAAACCGCCAGATACAGAAGAACTCGACCTTGAGACAGAAAAC |
| | | ATGGAAAGTATTCCTCTCGATCCTTCCTCAACTTTGCAGCCGACGGATAAAC |
| | | ATTCTGGCCACAAGGCGGAAAATGGCCCGGGGGTTGTAGTACTCAGGGCAT |
| | | CATTTGAAGAAAGGCAGCCATACGCACATATGAATGGGGGTCGCAAGAAC |
| | | GAGCGGGCTTTGCCTCTTCCCCAATCTTCTGCATGCTAG |
| 108 | IGFR-R1353H | GAATTCATGAAATCTGGGGCTGGGGGAGGGACCCTCGCCGTATTCTGTGGG |
| | | CTTTTGTTGGCGTTCGCCGCACTCTGTCTCTGTCCGACCAATGGTGAAATAT |
| | | GCGGCCCGAATGTGGATATTAGAAACGATATTCACGAGCTCAAGAGGCTCG |
| | | AAAATTGTACTGTAGTGGAGGGGTTTCTGCAAATACTGCTTATCAGTAAGG |
| | | CGGAAGATTACCGCAACTTCCGCTTTCCTAAGCTGACTGTGATCACAGACTA |
| | | TCTCCTGCTGTTCAGAGTTGCCGGACTTGAATCCCTCTCAGATCTGTTTCCTA |
| | | ACCTCACAGTGATCAGGGGTAGAAACCTGTTCTACAACTATGCTCTCGTAAT |
| | | ATTTGAGATGACGAACCTTAAGGAGATTGGACTCCACAATCTTCGCAACAT |
| | | AACCAGGGGTGCTATAAGGATCGAAAAAAATAGCGACCTGTGCTATCTTTC |
| | | TACAGTAGACTGGAGTCTCATATTGGACGCAGTATCCAACAACTATATAGTT |
| | | GGCAACAAGCCTCCCAAAGAATGCGGTGATCTCTGTCCTGGAACTATGGAG |
| | | GAAAAACCTCTCTGCGAAAAGACTAGTATAAACAACGAGTACAACTATAGG |
| | | TGTTGGACTACCAACCACTGCCAGAAGATGTGCCCGAGCAGCTGCGGCAAG |
| | | CGCGCGTGCACCGATCAGAATGAATGCTGCCACCCCGAATGTCTTGGTAGC |
| | | TGTACGGCTCCCGACAATAACACAGCCTGCGTTGCGTGTCGCAATTACTATT |
| | | ATGAGGGTGTCTGTATGCCTACCTGTCCGCCTAACACATATAAGTTCGAGGG |
| | | GTGGCGCTGCGTGACAAAAGAATTTTGTTCCAAGGTCCCAGCAACGGAGAC |
| | | GTCTGACTATGAAAGGTTTGTAATTCATAATGATGAATGTATGGCGGAATG |
| | | CCCATCTGGATTTATCAGGAACGGTAGTCAGAGCATGTTTTGCTCCCCATGT |
| | | GAAGGCCCATGTCCCAAAATTTGTGAAGACGGGAAGACGAAGACAATAGA |
| | | TAGCGTCACTTCTGCTCAAATGCTTCAGGGATGTACCATCTTGAAAGGAAAT |
| | | CTCTTGATTAACATTCGCCGGGGTAATAATATAGCAAGTGAGCTTGAAAAC |
| | | TTTATGGGGCTCATAGAAACGGTAACTGGGTATGTCAAGATCCGCCATAGT |
| | | CATGCACTCGTGTCACTTTCATTCTTGAAGAATCTCCGCTATATACTCGGCG |
| | | AAGAGCAAGTTGACGGCAACTACTCATTTTACGTTCTCGATAATCATAATTT |
| | | GCAGCAGCTTTGGGACTGGAATCACCACAACTTGACCATTAAAGAAGGAAA |
| | | GATGTACTTCGCTTTCAATCCGAAACTTTGTGTATCCGAAATTTACCGCATG |
| | | GAAGAGGTGTCTGGAACTAAAGGACGCCAGTCAAAAGGAGATATAAATCC |
| | | CAGGAATAATGGAGAAAGGGCGTCCTGTGAGAGCCATATATTGAGATTCGT |
| | | GAGCAATACCACGCTGAAGAACCGGATAAAACTCACCTGGGAGAGATACA |
| | | GGCCCCCGGATTACCGGGACCTCATCTCTTTCACGGTTTATTACAAGGAGGC |
| | | CCCCTTCAAAAACGTCACAGAGTACGATGGGCAAGATGCCTGCGGCTCCAA |
| | | CTCTTGGAACATGGTCGACGTAGACTTGCCCCCTAACAAAGAGAATGACCC |
| | | TGGTATATTGCTTCAAGGACTTAAACCTTGGACGCAGTACGCCATATACGTC |
| | | AAAGCCGTGACCCTTACAATGATGGAAAACCACCACATCCACGGGGCTAAA |
| | | TCCGAGATTGTTTATATAAGGACAAATGCAGCCGTCCCCAGCATACCTCTCG |
| | | ACGTAATATCTGCCTCTAATAGCAGCAGCCAGCTCATTGTGAAGTGGAATC |
| | | CTCCTTCACTTCCCAACGGGAATCTGTCTTATTACATTGTCCGCTGGCAACA |
| | | ACAACCTCAGGATTCTTATTTGTATCGGCATAACTACTGTAGTAAAGACAAG |
| | | GTGCCGATTCGCCGGTACGCGGACGGTACTATTGACACGGAAGAAGCAACA |
| | | GAGCCAACAAAACCCGAAGGATGCGGCGGTGAAAAAGGTCCGTGTTGTGC |
| | | GTGCCCTAAGACCGAAGCTGAGAAGCAAGCTGAAAAGGAAGAAGCTGAAT |
| | | ACCGCAAGGTGTTTGAGAACTTCCTGCATAACTCCATCTTCGTTCCTCGCCC |
| | | GGATAGAAAGAGGCGCGACGTGTTCAGGATTGCTAACGCCACGCTCGCTAC |
| | | GAGGAATCGGAACATTACTGGTGCGGATCACTTCACCAATGCATCCGATGC |
| | | GGAAGAGTCCGAAGTAGAGTACCCATTTTTTGAGACGAAAGTCGACGGGAA |
| | | GGAGAGAACGGTAATATCTCATTTGCAGCCTTTTACCCTTTATAGAATAGAT |
| | | ATCCACAGCTGCAATCACGAAGCAGACACCCTTGGTTGTAGTGCTTCTAACT |
| | | TCGTGTTTGCCCGCACGATGCCCTCTGAAGGCGCTGACAATATACCCGGAA |
| | | CCGTAGCATGGGAAGCGAAGGAGGAGAACACGGTTTATCTTAAATGGTTGG |
| | | AACCCACCAACCCTAACGGACTTATCTTGATGTACGAGATTAAATATGGAC |
| | | AACACGGGGAGGAAAAGAGGGAGTGTGTCAGTAGGCAAGAATACAAAAAA |
| | | CTTGGTGGCGCCAAATTGACACACTTGAACCCTGGTAATTACAGCGCGCGC |
| | | GTGCAGGCGACTAGCTTGGCAGGTAATGGTTCTTGGACTGAACCCGTCTCCT |
| | | TCTATGTTCAGCCCAAATCCGCAAATTACGACAACTTTCTCCATTTGATAAT |
| | | AGTCCTTCCAATAGCCTTCCTGTTGATAATAGGTGGCCTCCTCATTATGTTGT |
| | | ACGTCTTTAATAAGAAACGGAACTCTGACCGGCTCGGAAATGGTGTTCTGT |
| | | ATGCAAGTGTTAATCCAGAATATTTCTCTGCATCAGACGTTTATGTGCCGGA |
| | | CGAGTGGGAAGTGCCAAGGGAGAAAATTACAATGTGCAGAGAACTCGGAC |
| | | AGGGTTCTTTTGGCATGGTTTACGAGGGCATCGCAAAAGGCGTGGTGAAGG |
| | | ACGAACCCGAGACGAGAGTTGCTATCAAAACAGTCAATGAGTCAGCTTCTA |
| | | TGCGCGAACGGATTGAGTTTTTGAATGAGGCCAGTGTAATGAAGGAGTTTA |
| | | ACTGCCACCACGTCGTACGGTTGCTTGGCGTCGTGTCTCAAGGACAGCCCAC |
| | | CCTCGTTATTATGGAGTTGATGACCCGGGGGGATCTCAAGTCCTACTTGAGG |
| | | TCTCTTCGCCCTGACACTGAGTCAAACCCAGGGCAGGCACCTCCTACGCTGA |
| | | AAAAAATGATACAAATGGCTGGAGAAATCGCCGATGGGATGGCGTATCTCA |
| | | ACGCTAATAAGTTTGTACACCGCGATCTGGCCGCAAGAAATTGTATGGTCG |
| | | CAGAGGATTTTACTGTCAAGATCGGCGATTTCGGCATGACACGCGACATCT |
| | | ATGAGACCGACTACTATAGGAAGGGGGGGAAGGGTTTGCTCCCAGTAAGGT |
| | | GGATGTCACCGGAGAGCCTCAAGGATGGGGTGTTCACGACTCACAGCGATG |
| | | TCTGGTCTTTTGGAGTTGTACTCTGGGAAATAGCTACACTGGCAGAGCAACC |
| | | CTACCAAGGAATGACAAATGAACAAGTCCTCCGCTTTGTAATGGAAGGGGG |
| | | TCTTCTCGAAAAACCGGACAACTGCCCAGACATGCTTTTCGAGCTGATGCGC |
| | | ATGTGCTGGCAGTACAATCCCAAAATGCGCCCGTCTTTTCTTGAAATAATCT |
| | | CAAGCATCAAGGATGAACTTGACCCGGCCTTTAAAGAAGTATCATTTTTCTA |
| | | TTCCGAAGAAAATAAACCGCCAGATACAGAAGAACTCGACCTTGAGACAG |
| | | AAAACATGGAAAGTATTCCTCTCGATCCTTCCTCAACTTTGCAGCCGACGGA |
| | | TAAACATTCTGGCCACAAGGCGGAAAATGGCCCGGGGGTTGTAGTACTCAG |
| | | GGCATCATTTGAAGAAAGGCAGCCATACGCACATATGAATGGGGGTCGCAA |
| | | GAACGAGCATGCTTTGCCTCTTCCCCAATCTTCTGCATGCTAG |
| 109 | PDGFRa(WT) | ATGGGTACTCCCCCAAGGACGTTCCTGATCCTGGGATGTTTTCTCACAGGAC |
| | | CGCTCCTAACACTTTGCCAGCTTCCTCTGCCGACTATTGTTCCCAATAGAAA |
| | | TGAGATGGTTGTACAGCTGAATTCCAATTTCACACTCAAATGCTCTGGAGAC |
| | | AGCGAAGTGAGCTGGCAGTACCCAGTGACCGAGGGAAGCCACAGGATAGA |
| | | CATCAGACACGAGGAGAACAACAGTGGCCTCTTCGTGACAGTGCTTGAAGT |
| | | CGGAAATGCCTCAGCCGCTCACACGGGCATGTATGTTTGCTATTATAACCAC |
| | | ACGCAAGTGGAGGATGGGGAAGTCGAGGGGAAGGACATCTACATCTATGT |
| | | GCCTGACCCAGACATGCCTTTCGTTCCTTCCTTACCAGAAGACCAGTTCATC |
| | | CTAGTAGAAGAAGGTGATCCCACTGTTATCCCTTGTCGGACAAGTGACCCA |
| | | AGTGCTGAAGTGACTTTAGTTAACAGTTTAGACAAGCCTGTCTATGCTTTCT |
| | | ATGACAGCAAACAGGGCTTCGTAGGGAACTTCCTTGCAGGACCATACACAT |
| | | GCAAAACAATGGTTAAAGGCGTGGAGTTCAAGTCCGATGAGTTCCTCATCT |
| | | ATATTTTAAGAGCTACTTCACAGCTGCCGGTTGAAATTGAAGCTCTGAAAAC |
| | | TGTCTACAAAACAGGCGAGACCATCGTAGTAACTTGTGTGGTCTTTGACAAT |
| | | GAGGTGGTTAATTTACAGTGGAATTATCCCGGGAAAGTGAAAGAAAAAGGT |
| | | CTGATAAAACTTGATGATATCAAAGTCCCATCACAGAAGTTGGTTTACACGT |
| | | TGACCATACCTGACGCATCAGTGAAAGACACAGGGGATTATGAATGTACTG |
| | | CCCGACATGCAACCAAGGAGGTTAAGGAAAATAAGAAAGTAGTCATTACA |
| | | GTTCATGACAAAGGGTTCATTCATCTAGAGCCTCAATTTAGCCCTTTGGAAG |
| | | CTGTCAATCTACATGAAGTCAAAAATTTTGTCGTCGATGTGCAGGCGTACCC |
| | | CGCTCCAAAAATGTACTGGTTGAAGGATAATGTGACTCTGATTGAAAACCT |
| | | TACTGAGATTGTTACTAGTTCAAACAGAGTCCAGGAAACACGGTTTCAAAG |
| | | TGTACTAAAATTGATCCGGGCCAAGGAAGAAGACAGTGGGTACTATACTTT |
| | | GGTTGCTGAAAATGAAGATGAGATTAAGAGATACACCTTCTCGTTGCTAAT |
| | | ACAAGTTCCAGCCTTGATCTTAGACCTCATGGACGACCACCAAGGCTCTGCT |
| | | GGCAGGCAGACGGTGAGGTGCTTGGCTGAAGGTACCCCGCTTCCTGATGTG |
| | | GAATGGTTGGTTTGCAAGGACATTAAAAAATGCAGCAATGACACTTCCTGG |
| | | ACTCTTCTGACTAACAATATCTCTGATATACACATGGAAGCCCACCTGGATG |
| | | AGAGGAATATGGTGGAAAGCCAGGTGACCTTCCAGAAGGTAGAAGAAACC |
| | | CTGGCTGTGAGATGTGTAGCAAGAAACGACCTTGGAGCTGTTACTCGGGAA |
| | | CTGAAACTTGTGGCTCCCACCTTGCGATCAGAACTGACGGTGGCTGCTGCTG |
| | | TCTTAGTACTGCTGGTGATTGTGATAATTTCACTGATTGTCCTGGTCATCATC |
| | | TGGAAACAGAAGCCAAGATATGAGATAAGATGGAGAGTCATTGAGTCTATC |
| | | AGCCCCGATGGCCATGAATACATTTATGTGGACCCAATGCAGCTACCTTATG |
| | | ACTCCAGATGGGAGTTTCCTCGAGATGGGTTAGTGCTTGGTCGAATCCTTGG |
| | | TTCTGGTGCATTTGGAAAAGTGGTGGAAGGGACAGCATATGGATTGAGTCG |
| | | TTCTCAACCTGTGATGAAAGTAGCCGTGAAAATGCTGAAACCTACAGCTAG |
| | | ATCCAGTGAAAAACAGGCGCTCATGTCTGAACTGAAGATAATGACACATCT |
| | | TGGGCCCCACCTGAATATTGTGAACCTGCTTGGAGCTTGTACGAAATCAGGT |
| | | CCTATTTACATAATCACTGAATACTGCTTTTACGGTGATTTGGTGAACTATC |
| | | TGCACAAGAACAGGGACAACTTCCTCAGCCGACATCCAGAGAAACCAAAG |
| | | AAAGATCTGGATATTTTTGGGATGAACCCAGCTGATGAAAGCACAAGAAGC |
| | | TATGTGATTTTATCATTTGAAAACACCGGAGAATATATGGATATGAAACAA |
| | | GCTGATACCACTCAGTATGTGCCAATGCTGGAAAGGAAGGAGGGATCTAAA |
| | | TACTCTGATATTCAGAGATCTGTATATGATCGACCTGCTTCATATAAGAAGA |
| | | AATCTTTGTCAGAATCAGAAGTAAAAAACCTTCTTTCAGATGACGGTTCGG |
| | | AGGGTCTAAGCCTACTGGATTTGCTAAGCTTCACCTACCAGGTTGCACGGG |
| | | GAATGGAATTCTTGGCTTCTAAAAATTGCGTACACCGTGACTTGGCAGCTCG |
| | | TAATGTCCTTCTGGCTCAAGGCAAAATCGTGAAGATCTGCGACTTTGGGTTG |
| | | GCTAGAGACATCATGCATGATTCCAACTATGTCTCCAAGGGCAGCACCTTCC |
| | | TCCCAGTAAAATGGATGGCACCTGAAAGCATTTTTGACAATCTGTACACAA |
| | | CATTAAGTGATGTCTGGTCTTATGGCATTCTGCTGTGGGAAATATTTTCTCTT |
| | | GGTGGCACACCATATCCTGGCATGATGGTCGACTCCACCTTCTACAATAAG |
| | | ATAAAGAGTGGCTACCGAATGGCAAAACCTGATCATGCTACCAATGAAGTG |
| | | TATGAGATCATGGTAAAGTGCTGGAACAGTGAACCAGAGAAAAGACCTTCG |
| | | TTTTACCATCTGAGTGAAATTGTGGAGAGCTTGTTGCCTGGAGAGTACAAA |
| | | AAGAGCTACGAGAAGATTCACCTGGACTTCCTGAAAAGCGATCACCCAGCT |
| | | GTCACTCGAATGAGAGGGGACTGTGACAATGCTTACATTGGTGTCACCTAC |
| | | AAGAATGAAGACAAGATAAAGGATAGAGAGAGTGGATTTGATGAGCAGAG |
| | | GCTGAGTGCTGACAGTGGGTACATCATCCCCCTGCCTGACATTGACCCTGTT |
| | | TCTGAAGATGAGCTTGGCAAAAGGAACAGGCACAGTTCCCAGACATCTGAA |
| | | GAGAGTGCCATTGAAACCGGTTCCAGTAGCTCTACCTTTATAAAGAGAGAG |
| | | GATGAGACCATTGAGGACATTGACATGATGGATGACATTGGAATTGACTCC |
| | | TCGGATCTTGTAGAGGACAGCTTCCTGTAA |
| 110 | PDGFRa- | ATGGGTACTCCCCCAAGGACGTTCCTGATCCTGGGATGTTTTCTCACAGGAC |
| | D842V | CGCTCCTAACACTTTGCCAGCTTCCTCTGCCGACTATTGTTCCCAATAGAAA |
| | | TGAGATGGTTGTACAGCTGAATTCCAATTTCACACTCAAATGCTCTGGAGAC |
| | | AGCGAAGTGAGCTGGCAGTACCCAGTGACCGAGGGAAGCCACAGGATAGA |
| | | CATCAGACACGAGGAGAACAACAGTGGCCTCTTCGTGACAGTGCTTGAAGT |
| | | CGGAAATGCCTCAGCCGCTCACACGGGCATGTATGTTTGCTATTATAACCAC |
| | | ACGCAAGTGGAGGATGGGGAAGTCGAGGGGAAGGACATCTACATCTATGT |
| | | GCCTGACCCAGACATGCCTTTCGTTCCTTCCTTACCAGAAGACCAGTTCATC |
| | | CTAGTAGAAGAAGGTGATCCCACTGTTATCCCTTGTCGGACAAGTGACCCA |
| | | AGTGCTGAAGTGACTTTAGTTAACAGTTTAGACAAGCCTGTCTATGCTTTCT |
| | | ATGACAGCAAACAGGGCTTCGTAGGGAACTTCCTTGCAGGACCATACACAT |
| | | GCAAAACAATGGTTAAAGGCGTGGAGTTCAAGTCCGATGAGTTCCTCATCT |
| | | ATATTTTAAGAGCTACTTCACAGCTGCCGGTTGAAATTGAAGCTCTGAAAAC |
| | | TGTCTACAAAACAGGCGAGACCATCGTAGTAACTTGTGTGGTCTTTGACAAT |
| | | GAGGTGGTTAATTTACAGTGGAATTATCCCGGGAAAGTGAAAGAAAAAGGT |
| | | CTGATAAAACTTGATGATATCAAAGTCCCATCACAGAAGTTGGTTTACACGT |
| | | TGACCATACCTGACGCATCAGTGAAAGACACAGGGGATTATGAATGTACTG |
| | | CCCGACATGCAACCAAGGAGGTTAAGGAAAATAAGAAAGTAGTCATTACA |
| | | GTTCATGACAAAGGGTTCATTCATCTAGAGCCTCAATTTAGCCCTTTGGAAG |
| | | CTGTCAATCTACATGAAGTCAAAAATTTTGTCGTCGATGTGCAGGCGTACCC |
| | | CGCTCCAAAAATGTACTGGTTGAAGGATAATGTGACTCTGATTGAAAACCT |
| | | TACTGAGATTGTTACTAGTTCAAACAGAGTCCAGGAAACACGGTTTCAAAG |
| | | TGTACTAAAATTGATCCGGGCCAAGGAAGAAGACAGTGGGTACTATACTTT |
| | | GGTTGCTGAAAATGAAGATGAGATTAAGAGATACACCTTCTCGTTGCTAAT |
| | | ACAAGTTCCAGCCTTGATCTTAGACCTCATGGACGACCACCAAGGCTCTGCT |
| | | GGCAGGCAGACGGTGAGGTGCTTGGCTGAAGGTACCCCGCTTCCTGATGTG |
| | | GAATGGTTGGTTTGCAAGGACATTAAAAAATGCAGCAATGACACTTCCTGG |
| | | ACTCTTCTGACTAACAATATCTCTGATATACACATGGAAGCCCACCTGGATG |
| | | AGAGGAATATGGTGGAAAGCCAGGTGACCTTCCAGAAGGTAGAAGAAACC |
| | | CTGGCTGTGAGATGTGTAGCAAGAAACGACCTTGGAGCTGTTACTCGGGAA |
| | | CTGAAACTTGTGGCTCCCACCTTGCGATCAGAACTGACGGTGGCTGCTGCTG |
| | | TCTTAGTACTGCTGGTGATTGTGATAATTTCACTGATTGTCCTGGTCATCATC |
| | | TGGAAACAGAAGCCAAGATATGAGATAAGATGGAGAGTCATTGAGTCTATC |
| | | AGCCCCGATGGCCATGAATACATTTATGTGGACCCAATGCAGCTACCTTATG |
| | | ACTCCAGATGGGAGTTTCCTCGAGATGGGTTAGTGCTTGGTCGAATCCTTGG |
| | | TTCTGGTGCATTTGGAAAAGTGGTGGAAGGGACAGCATATGGATTGAGTCG |
| | | TTCTCAACCTGTGATGAAAGTAGCCGTGAAAATGCTGAAACCTACAGCTAG |
| | | ATCCAGTGAAAAACAGGCGCTCATGTCTGAACTGAAGATAATGACACATCT |
| | | TGGGCCCCACCTGAATATTGTGAACCTGCTTGGAGCTTGTACGAAATCAGGT |
| | | CCTATTTACATAATCACTGAATACTGCTTTTACGGTGATTTGGTGAACTATC |
| | | TGCACAAGAACAGGGACAACTTCCTCAGCCGACATCCAGAGAAACCAAAG |
| | | AAAGATCTGGATATTTTTGGGATGAACCCAGCTGATGAAAGCACAAGAAGC |
| | | TATGTGATTTTATCATTTGAAAACACCGGAGAATATATGGATATGAAACAA |
| | | GCTGATACCACTCAGTATGTGCCAATGCTGGAAAGGAAGGAGGGATCTAAA |
| | | TACTCTGATATTCAGAGATCTGTATATGATCGACCTGCTTCATATAAGAAGA |
| | | AATCTTTGTCAGAATCAGAAGTAAAAAACCTTCTTTCAGATGACGGTTCGG |
| | | AGGGTCTAAGCCTACTGGATTTGCTAAGCTTCACCTACCAGGTTGCACGGG |
| | | GAATGGAATTCTTGGCTTCTAAAAATTGCGTACACCGTGACTTGGCAGCTCG |
| | | TAATGTCCTTCTGGCTCAAGGCAAAATCGTGAAGATCTGCGACTTTGGGTTG |
| | | GCTAGAGTCATCATGCATGATTCCAACTATGTCTCCAAGGGCAGCACCTTCC |
| | | TCCCAGTAAAATGGATGGCACCTGAAAGCATTTTTGACAATCTGTACACAA |
| | | CATTAAGTGATGTCTGGTCTTATGGCATTCTGCTGTGGGAAATATTTTCTCTT |
| | | GGTGGCACACCATATCCTGGCATGATGGTCGACTCCACCTTCTACAATAAG |
| | | ATAAAGAGTGGCTACCGAATGGCAAAACCTGATCATGCTACCAATGAAGTG |
| | | TATGAGATCATGGTAAAGTGCTGGAACAGTGAACCAGAGAAAAGACCTTCG |
| | | TTTTACCATCTGAGTGAAATTGTGGAGAGCTTGTTGCCTGGAGAGTACAAA |
| | | AAGAGCTACGAGAAGATTCACCTGGACTTCCTGAAAAGCGATCACCCAGCT |
| | | GTCACTCGAATGAGAGGGGACTGTGACAATGCTTACATTGGTGTCACCTAC |
| | | AAGAATGAAGACAAGATAAAGGATAGAGAGAGTGGATTTGATGAGCAGAG |
| | | GCTGAGTGCTGACAGTGGGTACATCATCCCCCTGCCTGACATTGACCCTGTT |
| | | TCTGAAGATGAGCTTGGCAAAAGGAACAGGCACAGTTCCCAGACATCTGAA |
| | | GAGAGTGCCATTGAAACCGGTTCCAGTAGCTCTACCTTTATAAAGAGAGAG |
| | | GATGAGACCATTGAGGACATTGACATGATGGATGACATTGGAATTGACTCC |
| | | TCGGATCTTGTAGAGGACAGCTTCCTGTAA |
| 111 | PDGFRb(WT) | ATGCTCTGTCCCTCTCTGAAGGCATCTCTGCAGCTCCTCATCCTCACTGGTCT |
| | | GCTGGAGGTAACGTCTGGAGGCAGCGGGCTGCACATCGAACCTGAAGATGC |
| | | TGAGCTCGTCCTTAGGCTCCACAGCACTTTCTCCCTCGTGTGCTATGGGGAC |
| | | GGCACGCTGGTCTGGGAGCGGGATGGTCAGCCTCTCACTGCCGTGCTGGAG |
| | | CACAGGGACGGGGTCTTCATCAGCAACCTCACCCTCAGGAACGTGACAGGC |
| | | CGTCACACGGGGGAGTATGCGTGCTTCTACAGCCCTGACCAGGCTCCGGAG |
| | | CGAGCAGAGAGGAAAGCCCTTTACATCTATGTTCCAGATCCCTCCTTAGTTT |
| | | TTCTCCCCGCAATCACTTCTGAAGAGTTCTTCATCTTCATCACGGGCTACAC |
| | | AGAGGCCACCATCCCATGCCGTGTGACCAACCCAGAGCTGCAGGTGACCCT |
| | | CTATGAAAAGAAAGTGGAGAATCCCATTCCAGCTACTTATGACCCACAACA |
| | | GGGCTTCAAAGGCTTCTTTGAGGACAAGACCTACTACTGCCAGGCAATCGT |
| | | GGATGACCAAGAGGTGGATTCAGACACCTTCTATGTCTACCGGATCCAGGT |
| | | CTCATCTGTGAACGTCTCCATCAGCGCAGTGCAGACCGTAGTGCGGCAGGG |
| | | AGAAAATGTTACCCTGATGTGCACTGTCAGTGGCAATGAGCTGGTCAATTTC |
| | | AACTGGGATTATCCCCGCAAGCAGGCAGGGAAGGCTGTGGAGCCAGTGACC |
| | | GATTTCCTGCCTGGATCCACCCATGACATCCGTTCCATCCTCATCATCCAGA |
| | | ATGCAGAGCTAGAGGACAGTGGGACCTACGTCTGCAATGTCTCTGAGGGCT |
| | | ACCATGAGAAGACAGACCGGAAAGACATCACGGTCCAAGTGATCGAGCGT |
| | | GGCTTTGTACGCTTCCACACCCACCTGGCCAGCACGGTGTATGCTGAGGTCC |
| | | ACAAGAGCCACATCATCCAGGTGGATGTGGAGGCCTACCCACAGCCAAACA |
| | | TTGTGTGGCTGAAGAACAACAAGACATTGACCATGGAGAGCAGCAGCGAGT |
| | | TCACCATCACCAACAGGAACCTGTCAGAAACCAGGTATCAGACGTCTCTGG |
| | | TCCTGGTGCGTGTGAAGCAGGAGGAAGGAGGATATTACACCATCCGAGCTT |
| | | CCAATGAGGATGATGCACAAGAGCTGTCCTTCCATCTGCAGATAAATGTGC |
| | | CAGCCAAAGTGGTGGATCTCAAGGAAAACAGCAGTGCCAGCAGCGGGGAG |
| | | CAGACTGTAACGTGCTCTGCTGAAGGGATGCCCCAGCCAGAGATCAGTTGG |
| | | TCCACTTGCAGCAACATCAAATGGTGTGGCAGCCAGGGGCAACCCACCCAG |
| | | CTGCTGGGGAACAACTCTGCAGAGATTGGCCTGCACACTAATGCTACGTAC |
| | | CATGCAGAGCTGCAGGTGTACCGTGTGAACAGCACCCTGCAGCTGCACAGG |
| | | GTGGATGAACCCCTGCTTCTGAGATGCACCGTGCAAAACTTCCTGGGCTCCA |
| | | ACTCCCAAGACATCACTCTGGTCCCAAATGCCTTGCCATTCAAAGTGGTCAT |
| | | CATCTCCGTCATCCTGGCTCTGCTGGTCCTCACCGTCATCTCCCTGATCATCC |
| | | TGATCATCCTGTGGCAGAAGAAACCTCGCTATGAGATCCGCTGGAAGGTGA |
| | | TCGAGTCAGTGAGCTCCGATGGGCACGAGTACATCTACGTGGATCCCATGC |
| | | AGCTCCCTTATGACTCCAGCTGGGAGGTGCCCAGGGACAAGCTGGTGTTAG |
| | | GACGCACTCTTGGCTCCGGTGCCTTTGGACGCGTGGTGGAGGCAACAGCGC |
| | | ATGGCCTGAGCCATTCACAGTCCACCATGAAAGTGGCAGTCAAGATGCTCA |
| | | AGTCCACTGCACGGAGCAGTGAGAAGCAAGCCCTCATGTCTGAGCTGAAGA |
| | | TCATGAGCCACCTGGGACCTCACCTCAACATCGTCAACTTGCTGGGGGCCTG |
| | | CACCAAAGGAGGGCCCATCTATATCATCACCGAGTACTGCCGTTATGGGGA |
| | | CCTGGTGGACTACCTGCACCGCAACAAGCACACCTTCCTGCAGTCCTATGGC |
| | | GAGAAGGCCCGCCGGGAGGCAGAGCTGTATGGGAATACCATCAAGGAGGA |
| | | CCACGTGCAGAGTCACCTCTCCTTGTCTGTCGAGAGTGATGGGGGCTACATG |
| | | GACATGAGCAAGGATGAGTCTCTGGATTACGTGCCCATGTCTGACATGAAG |
| | | GGTGAAGTCAAGTATGCTGACATCGAGTCTTCTAACTATGGCACCCCATATG |
| | | AGCTGGACAGCTATTCCCCATCAGCTCCGGAAAGAACAGACCGGGTGACAC |
| | | TGATAAATGAATCTCCACTCCTCAGCTACATGGACTTGGTGGGCTTCAGCTT |
| | | CCAGGTGGCCAATGGGATGGAGTTCCTGGCTTCCAAAAATTGTGTGCATCG |
| | | TGACCTGGCTGCCAGGAACGTCCTCATCTGCGAGGGGAAGCTGGTGAAGAT |
| | | CTGTGACTTTGGTCTGGCAAGAGACATCATGAGGGATTCCAACTACATCTCC |
| | | AAAGGCAGTACCTTCTTGCCCCTTAAGTGGATGGCCCCAGAGAGCATCTTC |
| | | AACAACCTCTACACCACCCTAAGTGATGTGTGGTCCTTTGGGATTCTTCTCT |
| | | GGGAGATATTCACTCTAGGAGGGACTCCCTACCCTGAACTGCCTATGAACG |
| | | AACAGTTCTACAATGCCATCAAACGTGGCTATCGGATGTCCAAACCTACCC |
| | | ATGCTTCTGATGAAATCTACGATATCATGCAGAAGTGCTGGGAGGAGAAGT |
| | | TTGAGATCAGACCGTCCTTCTCACAGCTGGTGGTGCTTATGGGAAACCTCTT |
| | | GGTGGACTGCTACAGAAAGAGGTACCAACAGGTAGATGAAGAGTTCATGA |
| | | AGAGCGACCACCCCGCTGTTGTTCGCACAAGACCCACCATCCCCGGGCTGA |
| | | ACAACGCCAGGCTCCCTCCCAGCTCCCCCACCCTCTACACGGCTGTGCACCA |
| | | GAACGGGGGAGAGAACGACTACATCATCCCTCTTCCTGACCCCAAGCCTGA |
| | | TGCAATCTGTGACCTCCCTCAGGAGGCCTCCGTCAGCCGTGCCAGCTCTATG |
| | | CTGAATGAAGCCAACACATCATCTACAATATCCTGTGACAGCCCCCTGGGC |
| | | CCCCGGCAGGACGAGGAGCCAGAATGTGACCTGCAGCTGGGCTGCCAGGA |
| | | GCTGGCCCCGGGTCACCACGAGGTGGAGGAGAGCTTTCTGTAG |
| 112 | PDGFRb- | ATGCTCTGTCCCTCTCTGAAGGCATCTCTGCAGCTCCTCATCCTCACTGGTCT |
| | D850V | GCTGGAGGTAACGTCTGGAGGCAGCGGGCTGCACATCGAACCTGAAGATGC |
| | | TGAGCTCGTCCTTAGGCTCCACAGCACTTTCTCCCTCGTGTGCTATGGGGAC |
| | | GGCACGCTGGTCTGGGAGCGGGATGGTCAGCCTCTCACTGCCGTGCTGGAG |
| | | CACAGGGACGGGGTCTTCATCAGCAACCTCACCCTCAGGAACGTGACAGGC |
| | | CGTCACACGGGGGAGTATGCGTGCTTCTACAGCCCTGACCAGGCTCCGGAG |
| | | CGAGCAGAGAGGAAAGCCCTTTACATCTATGTTCCAGATCCCTCCTTAGTTT |
| | | TTCTCCCCGCAATCACTTCTGAAGAGTTCTTCATCTTCATCACGGGCTACAC |
| | | AGAGGCCACCATCCCATGCCGTGTGACCAACCCAGAGCTGCAGGTGACCCT |
| | | CTATGAAAAGAAAGTGGAGAATCCCATTCCAGCTACTTATGACCCACAACA |
| | | GGGCTTCAAAGGCTTCTTTGAGGACAAGACCTACTACTGCCAGGCAATCGT |
| | | GGATGACCAAGAGGTGGATTCAGACACCTTCTATGTCTACCGGATCCAGGT |
| | | CTCATCTGTGAACGTCTCCATCAGCGCAGTGCAGACCGTAGTGCGGCAGGG |
| | | AGAAAATGTTACCCTGATGTGCACTGTCAGTGGCAATGAGCTGGTCAATTTC |
| | | AACTGGGATTATCCCCGCAAGCAGGCAGGGAAGGCTGTGGAGCCAGTGACC |
| | | GATTTCCTGCCTGGATCCACCCATGACATCCGTTCCATCCTCATCATCCAGA |
| | | ATGCAGAGCTAGAGGACAGTGGGACCTACGTCTGCAATGTCTCTGAGGGCT |
| | | ACCATGAGAAGACAGACCGGAAAGACATCACGGTCCAAGTGATCGAGCGT |
| | | GGCTTTGTACGCTTCCACACCCACCTGGCCAGCACGGTGTATGCTGAGGTCC |
| | | ACAAGAGCCACATCATCCAGGTGGATGTGGAGGCCTACCCACAGCCAAACA |
| | | TTGTGTGGCTGAAGAACAACAAGACATTGACCATGGAGAGCAGCAGCGAGT |
| | | TCACCATCACCAACAGGAACCTGTCAGAAACCAGGTATCAGACGTCTCTGG |
| | | TCCTGGTGCGTGTGAAGCAGGAGGAAGGAGGATATTACACCATCCGAGCTT |
| | | CCAATGAGGATGATGCACAAGAGCTGTCCTTCCATCTGCAGATAAATGTGC |
| | | CAGCCAAAGTGGTGGATCTCAAGGAAAACAGCAGTGCCAGCAGCGGGGAG |
| | | CAGACTGTAACGTGCTCTGCTGAAGGGATGCCCCAGCCAGAGATCAGTTGG |
| | | TCCACTTGCAGCAACATCAAATGGTGTGGCAGCCAGGGGCAACCCACCCAG |
| | | CTGCTGGGGAACAACTCTGCAGAGATTGGCCTGCACACTAATGCTACGTAC |
| | | CATGCAGAGCTGCAGGTGTACCGTGTGAACAGCACCCTGCAGCTGCACAGG |
| | | GTGGATGAACCCCTGCTTCTGAGATGCACCGTGCAAAACTTCCTGGGCTCCA |
| | | ACTCCCAAGACATCACTCTGGTCCCAAATGCCTTGCCATTCAAAGTGGTCAT |
| | | CATCTCCGTCATCCTGGCTCTGCTGGTCCTCACCGTCATCTCCCTGATCATCC |
| | | TGATCATCCTGTGGCAGAAGAAACCTCGCTATGAGATCCGCTGGAAGGTGA |
| | | TCGAGTCAGTGAGCTCCGATGGGCACGAGTACATCTACGTGGATCCCATGC |
| | | AGCTCCCTTATGACTCCAGCTGGGAGGTGCCCAGGGACAAGCTGGTGTTAG |
| | | GACGCACTCTTGGCTCCGGTGCCTTTGGACGCGTGGTGGAGGCAACAGCGC |
| | | ATGGCCTGAGCCATTCACAGTCCACCATGAAAGTGGCAGTCAAGATGCTCA |
| | | AGTCCACTGCACGGAGCAGTGAGAAGCAAGCCCTCATGTCTGAGCTGAAGA |
| | | TCATGAGCCACCTGGGACCTCACCTCAACATCGTCAACTTGCTGGGGGCCTG |
| | | CACCAAAGGAGGGCCCATCTATATCATCACCGAGTACTGCCGTTATGGGGA |
| | | CCTGGTGGACTACCTGCACCGCAACAAGCACACCTTCCTGCAGTCCTATGGC |
| | | GAGAAGGCCCGCCGGGAGGCAGAGCTGTATGGGAATACCATCAAGGAGGA |
| | | CCACGTGCAGAGTCACCTCTCCTTGTCTGTCGAGAGTGATGGGGGCTACATG |
| | | GACATGAGCAAGGATGAGTCTCTGGATTACGTGCCCATGTCTGACATGAAG |
| | | GGTGAAGTCAAGTATGCTGACATCGAGTCTTCTAACTATGGCACCCCATATG |
| | | AGCTGGACAGCTATTCCCCATCAGCTCCGGAAAGAACAGACCGGGTGACAC |
| | | TGATAAATGAATCTCCACTCCTCAGCTACATGGACTTGGTGGGCTTCAGCTT |
| | | CCAGGTGGCCAATGGGATGGAGTTCCTGGCTTCCAAAAATTGTGTGCATCG |
| | | TGACCTGGCTGCCAGGAACGTCCTCATCTGCGAGGGGAAGCTGGTGAAGAT |
| | | CTGTGACTTTGGTCTGGCAAGAGACATCATGAGGAATTCCAACTACATCTCC |
| | | AAAGGCAGTACCTTCTTGCCCCTTAAGTGGATGGCCCCAGAGAGCATCTTC |
| | | AACAACCTCTACACCACCCTAAGTGATGTGTGGTCCTTTGGGATTCTTCTCT |
| | | GGGAGATATTCACTCTAGGAGGGACTCCCTACCCTGAACTGCCTATGAACG |
| | | AACAGTTCTACAATGCCATCAAACGTGGCTATCGGATGTCCAAACCTACCC |
| | | ATGCTTCTGATGAAATCTACGATATCATGCAGAAGTGCTGGGAGGAGAAGT |
| | | TTGAGATCAGACCGTCCTTCTCACAGCTGGTGGTGCTTATGGGAAACCTCTT |
| | | GGTGGACTGCTACAGAAAGAGGTACCAACAGGTAGATGAAGAGTTCATGA |
| | | AGAGCGACCACCCCGCTGTTGTTCGCACAAGACCCACCATCCCCGGGCTGA |
| | | ACAACGCCAGGCTCCCTCCCAGCTCCCCCACCCTCTACACGGCTGTGCACCA |
| | | GAACGGGGGAGAGAACGACTACATCATCCCTCTTCCTGACCCCAAGCCTGA |
| | | TGCAATCTGTGACCTCCCTCAGGAGGCCTCCGTCAGCCGTGCCAGCTCTATG |
| | | CTGAATGAAGCCAACACATCATCTACAATATCCTGTGACAGCCCCCTGGGC |
| | | CCCCGGCAGGACGAGGAGCCAGAATGTGACCTGCAGCTGGGCTGCCAGGA |
| | | GCTGGCCCCGGGTCACCACGAGGTGGAGGAGAGCTTTCTGTAG |
| 113 | PDGFRb- | ATGCTCTGTCCCTCTCTGAAGGCATCTCTGCAGCTCCTCATCCTCACTGGTCT |
| | V536A | GCTGGAGGTAACGTCTGGAGGCAGCGGGCTGCACATCGAACCTGAAGATGC |
| | | TGAGCTCGTCCTTAGGCTCCACAGCACTTTCTCCCTCGTGTGCTATGGGGAC |
| | | GGCACGCTGGTCTGGGAGCGGGATGGTCAGCCTCTCACTGCCGTGCTGGAG |
| | | CACAGGGACGGGGTCTTCATCAGCAACCTCACCCTCAGGAACGTGACAGGC |
| | | CGTCACACGGGGGAGTATGCGTGCTTCTACAGCCCTGACCAGGCTCCGGAG |
| | | CGAGCAGAGAGGAAAGCCCTTTACATCTATGTTCCAGATCCCTCCTTAGTTT |
| | | TTCTCCCCGCAATCACTTCTGAAGAGTTCTTCATCTTCATCACGGGCTACAC |
| | | AGAGGCCACCATCCCATGCCGTGTGACCAACCCAGAGCTGCAGGTGACCCT |
| | | CTATGAAAAGAAAGTGGAGAATCCCATTCCAGCTACTTATGACCCACAACA |
| | | GGGCTTCAAAGGCTTCTTTGAGGACAAGACCTACTACTGCCAGGCAATCGT |
| | | GGATGACCAAGAGGTGGATTCAGACACCTTCTATGTCTACCGGATCCAGGT |
| | | CTCATCTGTGAACGTCTCCATCAGCGCAGTGCAGACCGTAGTGCGGCAGGG |
| | | AGAAAATGTTACCCTGATGTGCACTGTCAGTGGCAATGAGCTGGTCAATTTC |
| | | AACTGGGATTATCCCCGCAAGCAGGCAGGGAAGGCTGTGGAGCCAGTGACC |
| | | GATTTCCTGCCTGGATCCACCCATGACATCCGTTCCATCCTCATCATCCAGA |
| | | ATGCAGAGCTAGAGGACAGTGGGACCTACGTCTGCAATGTCTCTGAGGGCT |
| | | ACCATGAGAAGACAGACCGGAAAGACATCACGGTCCAAGTGATCGAGCGT |
| | | GGCTTTGTACGCTTCCACACCCACCTGGCCAGCACGGTGTATGCTGAGGTCC |
| | | ACAAGAGCCACATCATCCAGGTGGATGTGGAGGCCTACCCACAGCCAAACA |
| | | TTGTGTGGCTGAAGAACAACAAGACATTGACCATGGAGAGCAGCAGCGAGT |
| | | TCACCATCACCAACAGGAACCTGTCAGAAACCAGGTATCAGACGTCTCTGG |
| | | TCCTGGTGCGTGTGAAGCAGGAGGAAGGAGGATATTACACCATCCGAGCTT |
| | | CCAATGAGGATGATGCACAAGAGCTGTCCTTCCATCTGCAGATAAATGTGC |
| | | CAGCCAAAGTGGTGGATCTCAAGGAAAACAGCAGTGCCAGCAGCGGGGAG |
| | | CAGACTGTAACGTGCTCTGCTGAAGGGATGCCCCAGCCAGAGATCAGTTGG |
| | | TCCACTTGCAGCAACATCAAATGGTGTGGCAGCCAGGGGCAACCCACCCAG |
| | | CTGCTGGGGAACAACTCTGCAGAGATTGGCCTGCACACTAATGCTACGTAC |
| | | CATGCAGAGCTGCAGGTGTACCGTGTGAACAGCACCCTGCAGCTGCACAGG |
| | | GTGGATGAACCCCTGCTTCTGAGATGCACCGTGCAAAACTTCCTGGGCTCCA |
| | | ACTCCCAAGACATCACTCTGGTCCCAAATGCCTTGCCATTCAAAGTGGTCAT |
| | | CATCTCCGTCATCCTGGCTCTGCTGGTCCTCACCGTCATCTCCCTGATCATCC |
| | | TGATCATCCTGTGGCAGAAGAAACCTCGCTATGAGATCCGCTGGAAGGCCA |
| | | TCGAGTCAGTGAGCTCCGATGGGCACGAGTACATCTACGTGGATCCCATGC |
| | | AGCTCCCTTATGACTCCAGCTGGGAGGTGCCCAGGGACAAGCTGGTGTTAG |
| | | GACGCACTCTTGGCTCCGGTGCCTTTGGACGCGTGGTGGAGGCAACAGCGC |
| | | ATGGCCTGAGCCATTCACAGTCCACCATGAAAGTGGCAGTCAAGATGCTCA |
| | | AGTCCACTGCACGGAGCAGTGAGAAGCAAGCCCTCATGTCTGAGCTGAAGA |
| | | TCATGAGCCACCTGGGACCTCACCTCAACATCGTCAACTTGCTGGGGGCCTG |
| | | CACCAAAGGAGGGCCCATCTATATCATCACCGAGTACTGCCGTTATGGGGA |
| | | CCTGGTGGACTACCTGCACCGCAACAAGCACACCTTCCTGCAGTCCTATGGC |
| | | GAGAAGGCCCGCCGGGAGGCAGAGCTGTATGGGAATACCATCAAGGAGGA |
| | | CCACGTGCAGAGTCACCTCTCCTTGTCTGTCGAGAGTGATGGGGGCTACATG |
| | | GACATGAGCAAGGATGAGTCTCTGGATTACGTGCCCATGTCTGACATGAAG |
| | | GGTGAAGTCAAGTATGCTGACATCGAGTCTTCTAACTATGGCACCCCATATG |
| | | AGCTGGACAGCTATTCCCCATCAGCTCCGGAAAGAACAGACCGGGTGACAC |
| | | TGATAAATGAATCTCCACTCCTCAGCTACATGGACTTGGTGGGCTTCAGCTT |
| | | CCAGGTGGCCAATGGGATGGAGTTCCTGGCTTCCAAAAATTGTGTGCATCG |
| | | TGACCTGGCTGCCAGGAACGTCCTCATCTGCGAGGGGAAGCTGGTGAAGAT |
| | | CTGTGACTTTGGTCTGGCAAGAGACATCATGAGGGATTCCAACTACATCTCC |
| | | AAAGGCAGTACCTTCTTGCCCCTTAAGTGGATGGCCCCAGAGAGCATCTTC |
| | | AACAACCTCTACACCACCCTAAGTGATGTGTGGTCCTTTGGGATTCTTCTCT |
| | | GGGAGATATTCACTCTAGGAGGGACTCCCTACCCTGAACTGCCTATGAACG |
| | | AACAGTTCTACAATGCCATCAAACGTGGCTATCGGATGTCCAAACCTACCC |
| | | ATGCTTCTGATGAAATCTACGATATCATGCAGAAGTGCTGGGAGGAGAAGT |
| | | TTGAGATCAGACCGTCCTTCTCACAGCTGGTGGTGCTTATGGGAAACCTCTT |
| | | GGTGGACTGCTACAGAAAGAGGTACCAACAGGTAGATGAAGAGTTCATGA |
| | | AGAGCGACCACCCCGCTGTTGTTCGCACAAGACCCACCATCCCCGGGCTGA |
| | | ACAACGCCAGGCTCCCTCCCAGCTCCCCCACCCTCTACACGGCTGTGCACCA |
| | | GAACGGGGGAGAGAACGACTACATCATCCCTCTTCCTGACCCCAAGCCTGA |
| | | TGCAATCTGTGACCTCCCTCAGGAGGCCTCCGTCAGCCGTGCCAGCTCTATG |
| | | CTGAATGAAGCCAACACATCATCTACAATATCCTGTGACAGCCCCCTGGGC |
| | | CCCCGGCAGGACGAGGAGCCAGAATGTGACCTGCAGCTGGGCTGCCAGGA |
| | | GCTGGCCCCGGGTCACCACGAGGTGGAGGAGAGCTTTCTGTAG |
| 114 | 9C3-PDGFRb | ATGGCGAAAGGTGGCATAATCGTCGCGATCCTTCTCCTTATCGTTATGCTTG |
| | | CTATAGAGATATTGCTTTTGATAACTCTTATCATTGCTGTAACGTCTGGAGG |
| | | CAGCGGGCTGCACATCGAACCTGAAGATGCTGAGCTCGTCCTTAGGCTCCA |
| | | CAGCACTTTCTCCCTCGTGTGCTATGGGGACGGCACGCTGGTCTGGGAGCG |
| | | GGATGGTCAGCCTCTCACTGCCGTGCTGGAGCACAGGGACGGGGTCTTCAT |
| | | CAGCAACCTCACCCTCAGGAACGTGACAGGCCGTCACACGGGGGAGTATGC |
| | | GTGCTTCTACAGCCCTGACCAGGCTCCGGAGCGAGCAGAGAGGAAAGCCCT |
| | | TTACATCTATGTTCCAGATCCCTCCTTAGTTTTTCTCCCCGCAATCACTTCTG |
| | | AAGAGTTCTTCATCTTCATCACGGGCTACACAGAGGCCACCATCCCATGCCG |
| | | TGTGACCAACCCAGAGCTGCAGGTGACCCTCTATGAAAAGAAAGTGGAGAA |
| | | TCCCATTCCAGCTACTTATGACCCACAACAGGGCTTCAAAGGCTTCTTTGAG |
| | | GACAAGACCTACTACTGCCAGGCAATCGTGGATGACCAAGAGGTGGATTCA |
| | | GACACCTTCTATGTCTACCGGATCCAGGTCTCATCTGTGAACGTCTCCATCA |
| | | GCGCAGTGCAGACCGTAGTGCGGCAGGGAGAAAATGTTACCCTGATGTGCA |
| | | CTGTCAGTGGCAATGAGCTGGTCAATTTCAACTGGGATTATCCCCGCAAGC |
| | | AGGCAGGGAAGGCTGTGGAGCCAGTGACCGATTTCCTGCCTGGATCCACCC |
| | | ATGACATCCGTTCCATCCTCATCATCCAGAATGCAGAGCTAGAGGACAGTG |
| | | GGACCTACGTCTGCAATGTCTCTGAGGGCTACCATGAGAAGACAGACCGGA |
| | | AAGACATCACGGTCCAAGTGATCGAGCGTGGCTTTGTACGCTTCCACACCC |
| | | ACCTGGCCAGCACGGTGTATGCTGAGGTCCACAAGAGCCACATCATCCAGG |
| | | TGGATGTGGAGGCCTACCCACAGCCAAACATTGTGTGGCTGAAGAACAACA |
| | | AGACATTGACCATGGAGAGCAGCAGCGAGTTCACCATCACCAACAGGAACC |
| | | TGTCAGAAACCAGGTATCAGACGTCTCTGGTCCTGGTGCGTGTGAAGCAGG |
| | | AGGAAGGAGGATATTACACCATCCGAGCTTCCAATGAGGATGATGCACAAG |
| | | AGCTGTCCTTCCATCTGCAGATAAATGTGCCAGCCAAAGTGGTGGATCTCA |
| | | AGGAAAACAGCAGTGCCAGCAGCGGGGAGCAGACTGTAACGTGCTCTGCT |
| | | GAAGGGATGCCCCAGCCAGAGATCAGTTGGTCCACTTGCAGCAACATCAAA |
| | | TGGTGTGGCAGCCAGGGGCAACCCACCCAGCTGCTGGGGAACAACTCTGCA |
| | | GAGATTGGCCTGCACACTAATGCTACGTACCATGCAGAGCTGCAGGTGTAC |
| | | CGTGTGAACAGCACCCTGCAGCTGCACAGGGTGGATGAACCCCTGCTTCTG |
| | | AGATGCACCGTGCAAAACTTCCTGGGCTCCAACTCCCAAGACATCACTCTG |
| | | GTCCCAAATGCCTTGCCATTCAAAGTGGTCATCATCTCCGTCATCCTGGCTC |
| | | TGCTGGTCCTCACCGTCATCTCCCTGATCATCCTGATCATCCTGTGGCAGAA |
| | | GAAACCTCGCTATGAGATCCGCTGGAAGGTGATCGAGTCAGTGAGCTCCGA |
| | | TGGGCACGAGTACATCTACGTGGATCCCATGCAGCTCCCTTATGACTCCAGC |
| | | TGGGAGGTGCCCAGGGACAAGCTGGTGTTAGGACGCACTCTTGGCTCCGGT |
| | | GCCTTTGGACGCGTGGTGGAGGCAACAGCGCATGGCCTGAGCCATTCACAG |
| | | TCCACCATGAAAGTGGCAGTCAAGATGCTCAAGTCCACTGCACGGAGCAGT |
| | | GAGAAGCAAGCCCTCATGTCTGAGCTGAAGATCATGAGCCACCTGGGACCT |
| | | CACCTCAACATCGTCAACTTGCTGGGGGCCTGCACCAAAGGAGGGCCCATC |
| | | TATATCATCACCGAGTACTGCCGTTATGGGGACCTGGTGGACTACCTGCACC |
| | | GCAACAAGCACACCTTCCTGCAGTCCTATGGCGAGAAGGCCCGCCGGGAGG |
| | | CAGAGCTGTATGGGAATACCATCAAGGAGGACCACGTGCAGAGTCACCTCT |
| | | CCTTGTCTGTCGAGAGTGATGGGGGCTACATGGACATGAGCAAGGATGAGT |
| | | CTCTGGATTACGTGCCCATGTCTGACATGAAGGGTGAAGTCAAGTATGCTG |
| | | ACATCGAGTCTTCTAACTATGGCACCCCATATGAGCTGGACAGCTATTCCCC |
| | | ATCAGCTCCGGAAAGAACAGACCGGGTGACACTGATAAATGAATCTCCACT |
| | | CCTCAGCTACATGGACTTGGTGGGCTTCAGCTTCCAGGTGGCCAATGGGAT |
| | | GGAGTTCCTGGCTTCCAAAAATTGTGTGCATCGTGACCTGGCTGCCAGGAA |
| | | CGTCCTCATCTGCGAGGGGAAGCTGGTGAAGATCTGTGACTTTGGTCTGGC |
| | | AAGAGACATCATGAGGGATTCCAACTACATCTCCAAAGGCAGTACCTTCTT |
| | | GCCCCTTAAGTGGATGGCCCCAGAGAGCATCTTCAACAACCTCTACACCAC |
| | | CCTAAGTGATGTGTGGTCCTTTGGGATTCTTCTCTGGGAGATATTCACTCTA |
| | | GGAGGGACTCCCTACCCTGAACTGCCTATGAACGAACAGTTCTACAATGCC |
| | | ATCAAACGTGGCTATCGGATGTCCAAACCTACCCATGCTTCTGATGAAATCT |
| | | ACGATATCATGCAGAAGTGCTGGGAGGAGAAGTTTGAGATCAGACCGTCCT |
| | | TCTCACAGCTGGTGGTGCTTATGGGAAACCTCTTGGTGGACTGCTACAGAA |
| | | AGAGGTACCAACAGGTAGATGAAGAGTTCATGAAGAGCGACCACCCCGCT |
| | | GTTGTTCGCACAAGACCCACCATCCCCGGGCTGAACAACGCCAGGCTCCCT |
| | | CCCAGCTCCCCCACCCTCTACACGGCTGTGCACCAGAACGGGGGAGAGAAC |
| | | GACTACATCATCCCTCTTCCTGACCCCAAGCCTGATGCAATCTGTGACCTCC |
| | | CTCAGGAGGCCTCCGTCAGCCGTGCCAGCTCTATGCTGAATGAAGCCAACA |
| | | CATCATCTACAATATCCTGTGACAGCCCCCTGGGCCCCCGGCAGGACGAGG |
| | | AGCCAGAATGTGACCTGCAGCTGGGCTGCCAGGAGCTGGCCCCGGGTCACC |
| | | ACGAGGTGGAGGAGAGCTTTCTGTAG |
| 115 | (F)-9C3- | ATGGCGGATTACAAGGATGATGATGACAAAAAAGGTGGCATAATCGTCGCG |
| | PDGFRb | ATCCTTCTCCTTATCGTTATGCTTGCTATAGAGATATTGCTTTTGATAACTCT |
| | | TATCATTGCTGTAACGTCTGGAGGCAGCGGGCTGCACATCGAACCTGAAGA |
| | | TGCTGAGCTCGTCCTTAGGCTCCACAGCACTTTCTCCCTCGTGTGCTATGGG |
| | | GACGGCACGCTGGTCTGGGAGCGGGATGGTCAGCCTCTCACTGCCGTGCTG |
| | | GAGCACAGGGACGGGGTCTTCATCAGCAACCTCACCCTCAGGAACGTGACA |
| | | GGCCGTCACACGGGGGAGTATGCGTGCTTCTACAGCCCTGACCAGGCTCCG |
| | | GAGCGAGCAGAGAGGAAAGCCCTTTACATCTATGTTCCAGATCCCTCCTTA |
| | | GTTTTTCTCCCCGCAATCACTTCTGAAGAGTTCTTCATCTTCATCACGGGCTA |
| | | CACAGAGGCCACCATCCCATGCCGTGTGACCAACCCAGAGCTGCAGGTGAC |
| | | CCTCTATGAAAAGAAAGTGGAGAATCCCATTCCAGCTACTTATGACCCACA |
| | | ACAGGGCTTCAAAGGCTTCTTTGAGGACAAGACCTACTACTGCCAGGCAAT |
| | | CGTGGATGACCAAGAGGTGGATTCAGACACCTTCTATGTCTACCGGATCCA |
| | | GGTCTCATCTGTGAACGTCTCCATCAGCGCAGTGCAGACCGTAGTGCGGCA |
| | | GGGAGAAAATGTTACCCTGATGTGCACTGTCAGTGGCAATGAGCTGGTCAA |
| | | TTTCAACTGGGATTATCCCCGCAAGCAGGCAGGGAAGGCTGTGGAGCCAGT |
| | | GACCGATTTCCTGCCTGGATCCACCCATGACATCCGTTCCATCCTCATCATC |
| | | CAGAATGCAGAGCTAGAGGACAGTGGGACCTACGTCTGCAATGTCTCTGAG |
| | | GGCTACCATGAGAAGACAGACCGGAAAGACATCACGGTCCAAGTGATCGA |
| | | GCGTGGCTTTGTACGCTTCCACACCCACCTGGCCAGCACGGTGTATGCTGAG |
| | | GTCCACAAGAGCCACATCATCCAGGTGGATGTGGAGGCCTACCCACAGCCA |
| | | AACATTGTGTGGCTGAAGAACAACAAGACATTGACCATGGAGAGCAGCAG |
| | | CGAGTTCACCATCACCAACAGGAACCTGTCAGAAACCAGGTATCAGACGTC |
| | | TCTGGTCCTGGTGCGTGTGAAGCAGGAGGAAGGAGGATATTACACCATCCG |
| | | AGCTTCCAATGAGGATGATGCACAAGAGCTGTCCTTCCATCTGCAGATAAA |
| | | TGTGCCAGCCAAAGTGGTGGATCTCAAGGAAAACAGCAGTGCCAGCAGCG |
| | | GGGAGCAGACTGTAACGTGCTCTGCTGAAGGGATGCCCCAGCCAGAGATCA |
| | | GTTGGTCCACTTGCAGCAACATCAAATGGTGTGGCAGCCAGGGGCAACCCA |
| | | CCCAGCTGCTGGGGAACAACTCTGCAGAGATTGGCCTGCACACTAATGCTA |
| | | CGTACCATGCAGAGCTGCAGGTGTACCGTGTGAACAGCACCCTGCAGCTGC |
| | | ACAGGGTGGATGAACCCCTGCTTCTGAGATGCACCGTGCAAAACTTCCTGG |
| | | GCTCCAACTCCCAAGACATCACTCTGGTCCCAAATGCCTTGCCATTCAAAGT |
| | | GGTCATCATCTCCGTCATCCTGGCTCTGCTGGTCCTCACCGTCATCTCCCTGA |
| | | TCATCCTGATCATCCTGTGGCAGAAGAAACCTCGCTATGAGATCCGCTGGA |
| | | AGGTGATCGAGTCAGTGAGCTCCGATGGGCACGAGTACATCTACGTGGATC |
| | | CCATGCAGCTCCCTTATGACTCCAGCTGGGAGGTGCCCAGGGACAAGCTGG |
| | | TGTTAGGACGCACTCTTGGCTCCGGTGCCTTTGGACGCGTGGTGGAGGCAA |
| | | CAGCGCATGGCCTGAGCCATTCACAGTCCACCATGAAAGTGGCAGTCAAGA |
| | | TGCTCAAGTCCACTGCACGGAGCAGTGAGAAGCAAGCCCTCATGTCTGAGC |
| | | TGAAGATCATGAGCCACCTGGGACCTCACCTCAACATCGTCAACTTGCTGG |
| | | GGGCCTGCACCAAAGGAGGGCCCATCTATATCATCACCGAGTACTGCCGTT |
| | | ATGGGGACCTGGTGGACTACCTGCACCGCAACAAGCACACCTTCCTGCAGT |
| | | CCTATGGCGAGAAGGCCCGCCGGGAGGCAGAGCTGTATGGGAATACCATCA |
| | | AGGAGGACCACGTGCAGAGTCACCTCTCCTTGTCTGTCGAGAGTGATGGGG |
| | | GCTACATGGACATGAGCAAGGATGAGTCTCTGGATTACGTGCCCATGTCTG |
| | | ACATGAAGGGTGAAGTCAAGTATGCTGACATCGAGTCTTCTAACTATGGCA |
| | | CCCCATATGAGCTGGACAGCTATTCCCCATCAGCTCCGGAAAGAACAGACC |
| | | GGGTGACACTGATAAATGAATCTCCACTCCTCAGCTACATGGACTTGGTGG |
| | | GCTTCAGCTTCCAGGTGGCCAATGGGATGGAGTTCCTGGCTTCCAAAAATTG |
| | | TGTGCATCGTGACCTGGCTGCCAGGAACGTCCTCATCTGCGAGGGGAAGCT |
| | | GGTGAAGATCTGTGACTTTGGTCTGGCAAGAGACATCATGAGGGATTCCAA |
| | | CTACATCTCCAAAGGCAGTACCTTCTTGCCCCTTAAGTGGATGGCCCCAGAG |
| | | AGCATCTTCAACAACCTCTACACCACCCTAAGTGATGTGTGGTCCTTTGGGA |
| | | TTCTTCTCTGGGAGATATTCACTCTAGGAGGGACTCCCTACCCTGAACTGCC |
| | | TATGAACGAACAGTTCTACAATGCCATCAAACGTGGCTATCGGATGTCCAA |
| | | ACCTACCCATGCTTCTGATGAAATCTACGATATCATGCAGAAGTGCTGGGA |
| | | GGAGAAGTTTGAGATCAGACCGTCCTTCTCACAGCTGGTGGTGCTTATGGG |
| | | AAACCTCTTGGTGGACTGCTACAGAAAGAGGTACCAACAGGTAGATGAAGA |
| | | GTTCATGAAGAGCGACCACCCCGCTGTTGTTCGCACAAGACCCACCATCCC |
| | | CGGGCTGAACAACGCCAGGCTCCCTCCCAGCTCCCCCACCCTCTACACGGCT |
| | | GTGCACCAGAACGGGGGAGAGAACGACTACATCATCCCTCTTCCTGACCCC |
| | | AAGCCTGATGCAATCTGTGACCTCCCTCAGGAGGCCTCCGTCAGCCGTGCC |
| | | AGCTCTATGCTGAATGAAGCCAACACATCATCTACAATATCCTGTGACAGC |
| | | CCCCTGGGCCCCCGGCAGGACGAGGAGCCAGAATGTGACCTGCAGCTGGGC |
| | | TGCCAGGAGCTGGCCCCGGGTCACCACGAGGTGGAGGAGAGCTTTCTGTAG |
| 116 | PDGF(WT) | ATGTGCCCGCAGCCGGCAAGGCTTGAACCCGGCATGAATTTCGGCGTGGTC |
| | | TTCGCCGTCATCCTCTCCCTGCCCCTGGCCCGCCTGGAGGGGGACCCCATAC |
| | | CCGAAGATATTTATGAGATTTTGGGTGGCAGCTCCGTGCGCTCCATCAGTGA |
| | | CCTCCAGCGTGCCCTGCGGATAGACTCCGTAGAGGAGGACAGCTCTAGCCT |
| | | GGACCTGAATGCAACTCAGCCCAGCCAAAACCATGTGTCCCTGTCTCGAGA |
| | | GAGGCGAAGCCTTGATGCTCTGGCAGCAGCAGAGCCAGCTGTCCTCGCCGA |
| | | GTGCAAGACACGGACGGTGGTCTTTGAGATCTCCCGTGACATGGTGGACAG |
| | | CACCAATGCCAACTTCGTGGTGTGGCCACCCTGCGTGGAGGTGCAGCGGTG |
| | | CTCCGGCTGCTGCAACAACCGCAACGTGCAGTGCCGCCCCATGCAGATTCG |
| | | CGTCCGGCATGTCCAGGTGAACAAGATTGAGTTTTTCCAGAGGAAGCCAAT |
| | | ATTCAAAAAAGTCATCGTGCCTTTGGAGGACCACGTGCAGTGCCGGTGCGA |
| | | AGTGGTGTCCCGGCCGCCACCCAGGAGCAACCGACCGGCATCCCGTGAGCA |
| | | GAGACGCTTCTCGCCGTCATTCACCACAGCCGCCATCTCCCAGAGGAAGCG |
| | | GGTACGCCGGCCGCCAGCACAGAAGAGAAAACACAAGAAATACAAGCATG |
| | | TCAACGATAAGAAAGTGCTGAAAGAAATCCTCATAGCATAG |
| 117 | PDGF-211* | ATGTGCCCGCAGCCGGCAAGGCTTGAACCCGGCATGAATTTCGGCGTGGTC |
| | | TTCGCCGTCATCCTCTCCCTGCCCCTGGCCCGCCTGGAGGGGGACCCCATAC |
| | | CCGAAGATATTTATGAGATTTTGGGTGGCAGCTCCGTGCGCTCCATCAGTGA |
| | | CCTCCAGCGTGCCCTGCGGATAGACTCCGTAGAGGAGGACAGCTCTAGCCT |
| | | GGACCTGAATGCAACTCAGCCCAGCCAAAACCATGTGTCCCTGTCTCGAGA |
| | | GAGGCGAAGCCTTGATGCTCTGGCAGCAGCAGAGCCAGCTGTCCTCGCCGA |
| | | GTGCAAGACACGGACGGTGGTCTTTGAGATCTCCCGTGACATGGTGGACAG |
| | | CACCAATGCCAACTTCGTGGTGTGGCCACCCTGCGTGGAGGTGCAGCGGTG |
| | | CTCCGGCTGCTGCAACAACCGCAACGTGCAGTGCCGCCCCATGCAGATTCG |
| | | CGTCCGGCATGTCCAGGTGAACAAGATTGAGTTTTTCCAGAGGAAGCCAAT |
| | | ATTCAAAAAAGTCATCGTGCCTTTGGAGGACCACGTGCAGTGCCGGTGCGA |
| | | AGTGGTGTCCCGGCCGCCACCCAGGAGCAACCGACCGGCATCCCGTGAGCA |
| | | GAGACGCTTCTCGCCGTCATTCACCACAGCCGCCATCTCCCAGTAG |
| 118 | 9C3-SRG | ATGGCGAAAGGTGGCATAATCGTCGCGATCCTTCTCCTTATCGTTATGCTTG |
| | | CTATAGAGATATTGCTTTTGATAACTCTTATCATTGCTGTAACGTCTGGAGG |
| | | CAGCGGGTAG |
| 119 | (F)9C3-SRG | ATGGCGGATTACAAGGATGATGATGACAAAAAAGGTGGCATAATCGTCGCG |
| | | ATCCTTCTCCTTATCGTTATGCTTGCTATAGAGATATTGCTTTTGATAACTCT |
| | | TATCATTGCTGTAACGTCTGGAGGCAGCGGGTAG |
| 120 | FC551A- | GCCCCTGCAGCCGAATTATATTATTTTTGCCAAATAATTTTTAACAAAAGCT |
| | empty vector | CTGAAGTCTTCTTCATTTAAATTCTTAGATGATACTTCATCTGGAAAATTGTC |
| | | CCAATTAGTAGCATCACGCTGTGAGTAAGTTCTAAACCATTTTTTTATTGTT |
| | | GTATTATCTCTAATCTTACTACTCGATGAGTTTTCGGTATTATCTCTATTTTT |
| | | AACTTGGAGCAGGTTCCATTCATTGTTTTTTTCATCATAGTGAATAAAATCA |
| | | ACTGCTTTAACACTTGTGCCTGAACACCATATCCATCCGGCGTAATACGACT |
| | | CACTATAGGGAGAGCGGCCGCGTCGACATGCCCGCCGTGACCGTCGAGAAC |
| | | CCGCTGACGCTGCCCCGCGTATCCGCACCCGCCGACGCCGTCGCACGTCCC |
| | | GTGCTCACCGTGACCACCGCGCCCAGCGGTTTCGAGGGCGAGGGCTTCCCG |
| | | GTGCGCCGCGCGTTCGCCGGGATCAACTACCGCCACCTCGACCCGTTCATCA |
| | | TGATGGACCAGATGGGTGAGGTGGAGTACGCGCCCGGGGAGCCCAAGGGC |
| | | ACGCCCTGGCACCCGCACCGCGGCTTCGAGACCGTGACCTACATCGTCGAC |
| | | GCGGCCGCCAGATCTTCCGGATGGCTCGAGTTTTTCAGCAAGATATGATAA |
| | | GATACATTGATGAGTTTGGACAAACCACAACTAGAATGCAGTGAAAAAAAT |
| | | GCTTTATTTGTGAAATTTGTGATGCTATTGCTTTATTTGTAACCATTATAAGC |
| | | TGCAATAAACAAGTTTAGTTAACGCATGATACAAAGGCATTAAAGCAGCGT |
| | | ATCCACATAGCGTAAAAGGAGCAACATAGTTAAGAATACCAGTCAATCTTT |
| | | CACAAATTTTGTAATCCAGAGGTTGATTTCAGGCACCGGGCTTGCGGGTCAT |
| | | GCACCAGGTGCGCGGTCCTTCGGGCACCTCGACGTCGGCGGTGACGGTGAA |
| | | GCCGAGCCGCTCGTAGAAGGGGAGGTTGCGGGGCGCGGAGGTCTCCAGGA |
| | | AGGCGGGCACCCCGGCGCGCTCGGCCGCCTCCACTCCGGGGAGCACGACGG |
| | | CGCTGCCCAGACCCTTGCCCTGGTGGTCGGGCGAGACGCCGACGGTGGCCA |
| | | GGAACCACGCGGGCTCCTTGGGCCGGTGCGGCGCCAGGAGGCCTTCCATCT |
| | | GTTGCTGCGCGGCCAGCCGGGAACCGCTCAACTCGGCCATGCGCGGGCCGA |
| | | TCTCGGCGAACACCGCCCCCGCTTCGACGCTCTCCGGCGTGGTCCAGACCGC |
| | | CACCGCGGCGCCGTCGTCCGCGACCCACACCTTGCCGATGTCGAGCCCGAC |
| | | GCGCGTGAGGAAGAGTTCTTGCAGCTCGGTGACCCGCTCGATGTGGCGGTC |
| | | CGGATCGACGGTGTGGCGCGTGGCGGGGTAGTCGGCGAACGCGGCGGCGA |
| | | GGGTGCGTACGGCCCTGGGGACGTCGTCGCGGGTGGCGAGGCGCACCGTGG |
| | | GCTTGTACTCGGTCATAGGGCCGGGATTCTCCTCCACGTCACCGCATGTTAG |
| | | AAGACTTCCTCTGCCCTCGCGAGATCCGGTGGAGCCGGGTCCGGCGGTGCC |
| | | GTCCACGGCAGAATTGGACGACTGAGCGCGGGATCTGGCGAAGGCGATGG |
| | | GGGTCTTGAAGGCGTGCTGGTACTCCACGATGCCCAGCTCGGTGTTGCTGTG |
| | | CAGCTCCTCCACGCGGCGGAAGGCGAACATGGGGCCCCCGTTCTGCAGGAT |
| | | GCTGGGGTGGATGGCGCTCTTGAAGTGCATGTGGCTGTCCACCACGAAGCT |
| | | GTAGTAGCCGCCGTCGCGCAGGCTGAAGGTGCGGGCGAAGCTGCCCACCAG |
| | | CACGTTATCGCCCATGGGGTGCAGGTGCTCCACGGTGGCGTTGCTGCGGAT |
| | | GATCTTGTCGGTGAAGATCACGCTGTCCTCGGGGAAGCCGGTGCCCACCAC |
| | | CTTGAAGTCGCCGATCACGCGGCCGGCCTCGTAGCGGTAGCTGAAGCTCAC |
| | | GTGCAGCACGCCGCCGTCCTCGTACTTCTCGATGCGGGTGTTGGTGTAGCCG |
| | | CCGTTGTTGATGGCGTGCAGGAAGGGGTTCTCGTAGCCGCTGGGGTAGGTG |
| | | CCGAAGTGGTAGAAGCCGTAGCCCATCACGTGGCTCAGCAGGTAGGGGCTG |
| | | AAGGTCAGGGCGCCTTTGGTGCTCTTCATCTTGTTGGTCATGCGGCCCTGCT |
| | | TGGGGGTGCCCTCTCCGCCGCCCACCAGCTCGAACTCCACGCCGTTCAGGGT |
| | | GCCGGTGATGCGGCACTCGATCTCCATGGCGGGCAGGCCGCTCTCGTCGCT |
| | | CTCCATGGTGGCGTCTAGCGTAGGCGCCGGTCACAGCTTGGATCTGTAACG |
| | | GCGCAGAACAGAAAACGAAACAAAGACGTAGAGTTGAGCAAGCAGGGTCA |
| | | GGCAAAGCGTGGAGAGCCGGCTGAGTCTAGGTAGGCTCCAAGGGAGCGCC |
| | | GGACAAAGGCCCGGTCTCGACCTGAGCTTTAAACTTACCTAGACGGCGGAC |
| | | GCAGTTCAGGAGGCACCACAGGCGGGAGGCGGCAGAACGCGACTCAACCG |
| | | GCGTGGATGGCGGCCTCAGGTAGGGCGGCGGGCGCGTGAAGGAGAGATGC |
| | | GAGCCCCTCGAAGCTTCAGCTGTGTTCTGGCGGCAAACCCGTTGCGAAAAA |
| | | GAACGTTCACGGCGACTACTGCACTTATATACGGTTCTCCCCCACCCTCGGG |
| | | AAAAAGGCGGAGCCAGTACACGACATCACTTTCCCAGTTTACCCCGCGCCA |
| | | CCTTCTCTAGGCACCCGTTCAATTGCCGACCCCTCCCCCCAACTTCTCGGGG |
| | | ACTGTGGGCGATGTGCGCTCTGCCCACTGACGGGCACCGGAGCGATCGCAG |
| | | ATCCTTATCTTTCTAGAAATTCTACCGGGTAGGGGAGGCGCTTTTCCCAAGG |
| | | CAGTCTGGAGCATGCGCTTTAGCAGCCCCGCTGGCACTTGGCGCTACACAA |
| | | GTGGCCTCTGGCCTCGCACACATTCCACATCCACCGGTAGCGCCAACCGGCT |
| | | CCGTTCTTTGGTGGCCCCTTCGCGCCACCTTCTACTCCTCCCCTAGTCAGGA |
| | | AGTTCCCCCCCGCCCCGCAGCTCGCGTCGTGCAGGACGTGACAAATGGAAG |
| | | TAGCACGTCTCACTAGTCTCGTGCAGATGGACAGCACCGCTGAGCAATGGA |
| | | AGCGGGTAGGCCTTTGGGGCAGCGGCCAATAGCAGCTTTGCTCCTTCGCTTT |
| | | CTGGGCTCAGCAGCTGGGAAGGGTGGGTCCGGGGGCGGGCTCAGGGGCGG |
| | | GCTCAGGGGCGGGGGGGGCGCCCGAAGGTCCTCCGGAGGCCCGGCATTCTG |
| | | CACGCTTCAAAAGCGCACGTCTGCCGCGCTGTTCTCCTCTTCCTCATCTCCG |
| | | GGCCTTTCGACCTGGATCCGATATCGGTACCGCTAGCATCGATCAGACATG |
| | | ATAAGATACATTGATGAGTTTGGACAAACCACAACTAGAATGCAGTGAAAA |
| | | AAATGCTTTATTTGTGAAATTTGTGATGCTATTGCTTTATTTGTAACCATTAT |
| | | AAGCTGCAATAAACAAGTTAACAACAACAATTGCATTCATTTTATGTTTCAG |
| | | GTTCAGGGGGAGGTGTGGGAGGTTTTTTGTTTAAACCTCCTGTGTGAAATTA |
| | | TTATCCGCTCATAATTCCACACATTATACGAGCCGGAAGCATAAAGTGTAA |
| | | AGCCTGGGGTGCCTAATGAGTGAGCTAACTCACATTAATTGCGTTGCGCTCA |
| | | CTGCCAATTGCTTTCCAGTCGGGAAACCTGTCGTGCCAGCTGCATTAATGAA |
| | | TCGGCCAACGCGCGGGGAGAGGCGGTTTGCGTATTGGGCGCTCTTCCGCTT |
| | | CCTCGCTCACTGACTCGCTGCGCTCGGTCGTTCGGCTGCGGCGAGCGGTATC |
| | | AGCTCACTCAAAGGCGGTAATACGGTTATCCACAGAATCAGGGGATAACGC |
| | | AGGAAAGAACATGTGAGCAAAAGGCCAGCAAAAGGCCAGGAACCGTAAAA |
| | | AGGCCGCGTTGCTGGCGTTTTTCCATAGGCTCCGCCCCCCTGACGAGCATCA |
| | | CAAAAATCGACGCTCAAGTCAGAGGTGGCGAAACCCGACAGGACTATAAA |
| | | GATACCAGGCGTTTCCCCCTGGAAGCTCCCTCGTGCGCTCTCCTGTTCCGAC |
| | | CCTGCCGCTTACCGGATACCTGTCCGCCTTTCTCCCTTCGGGAAGCGTGGCG |
| | | CTTTCTCATAGCTCACGCTGTAGGTATCTCAGTTCGGTGTAGGTCGTTCGCT |
| | | CCAAGCTGGGCTGTGTGCACGAACCCCCCGTTCAGCCCGACCGCTGCGCCTT |
| | | ATCCGGTAACTATCGTCTTGAGTCCAACCCGGTAAGACACGACTTATCGCCA |
| | | CTGGCAGCAGCCACTGGTAACAGGATTAGCAGAGCGAGGTATGTAGGCGGT |
| | | GCTACAGAGTTCTTGAAGTGGTGGCCTAACTACGGCTACACTAGAAGAACA |
| | | GTATTTGGTATCTGCGCTCTGCTGAAGCCAGTTACCTTCGGAAAAAGAGTTG |
| | | GTAGCTCTTGATCCGGCAAACAAACCACCGCTGGTAGCGGTGGTTTTTTTGT |
| | | TTGCAAGCAGCAGATTACGCGCAGAAAAAAAGGATCTCAAGAAGATCCTTT |
| | | GATCTTTTCTACGGGGTCTGACGCTCAGTGGAACGAAAACTCACGTTAAGG |
| | | GATTTTGGTCATGAGATTATCAAAAAGGATCTTCACCTAGATCCTTTTAAAT |
| | | TAAAAATGAAGTTTTAAATCAATCTAAAGTATATATGAGTAAACTTGGTCTG |
| | | ACAGTTACCAATGCTTAATCAGTGAGGCACCTATCTCAGCGATCTGTCTATT |
| | | TCGTTCATCCATAGTTGCCTGACTCCCCGTCGTGTAGATAACTACGATACGG |
| | | GAGGGCTTACCATCTGGCCCCAGTGCTGCAATGATACCGCGAGACCCACGC |
| | | TCACCGGCTCCAGATTTATCAGCAATAAACCAGCCAGCCGGAAGGGCCGAG |
| | | CGCAGAAGTGGTCCTGCAACTTTATCCGCCTCCATCCAGTCTATTAATTGTT |
| | | GCCGGGAAGCTAGAGTAAGTAGTTCGCCAGTTAATAGTTTGCGCAACGTTG |
| | | TTGCCATTGCTACAGGCATCGTGGTGTCACGCTCGTCGTTTGGTATGGCTTC |
| | | ATTCAGCTCCGGTTCCCAACGATCAAGGCGAGTTACATGATCCCCCATGTTG |
| | | TGCAAAAAAGCGGTTAGCTCCTTCGGTCCTCCGATCGTTGTCAGAAGTAAGT |
| | | TGGCCGCAGTGTTATCACTCATGGTTATGGCAGCACTGCATAATTCTCTTAC |
| | | TGTCATGCCATCCGTAAGATGCTTTTCTGTGACTGGTGAGTACTCAACCAAG |
| | | TCATTCTGAGAATAGTGTATGCGGCGACCGAGTTGCTCTTGCCCGGCGTCAA |
| | | TACGGGATAATACCGCGCCACATAGCAGAACTTTAAAAGTGCTCATCATTG |
| | | GAAAACGTTCTTCGGGGCGAAAACTCTCAAGGATCTTACCGCTGTTGAGAT |
| | | CCAGTTCGATGTAACCCACTCGTGCACCCAACTGATCTTCAGCATCTTTTAC |
| | | TTTCACCAGCGTTTCTGGGTGAGCAAAAACAGGAAGGCAAAATGCCGCAAA |
| | | AAAGGGAATAAGGGCGACACGGAAATGTTGAATACTCATACTCTTCCTTTT |
| | | TCAATATTATTGAAGCATTTATCAGGGTTATTGTCTCATGAGCGGATACATA |
| | | TTTGAATGTATTTAGAAAAATAAACAAATAGGGGTTCCGCGCACATTTCCCC |
| | | GAAAAGTGCCACCTGACGTCTAAGAAACCATTATTATCATGACATTAACCT |
| | | ATAAAAATAGGCGTATCACGAGGCC |
| 121 | FC550A- | GCCCCTGCAGCCGAATTATATTATTTTTGCCAAATAATTTTTAACAAAAGCT |
| | empty vector | CTGAAGTCTTCTTCATTTAAATTCTTAGATGATACTTCATCTGGAAAATTGTC |
| | (EVmRuby) | CCAATTAGTAGCATCACGCTGTGAGTAAGTTCTAAACCATTTTTTTATTGTT |
| | | GTATTATCTCTAATCTTACTACTCGATGAGTTTTCGGTATTATCTCTATTTTT |
| | | AACTTGGAGCAGGTTCCATTCATTGTTTTTTTCATCATAGTGAATAAAATCA |
| | | ACTGCTTTAACACTTGTGCCTGAACACCATATCCATCCGGCGTAATACGACT |
| | | CACTATAGGGAGAGCGGCCGCCAGATCTTCCGGATGGCTCGAGTTTTTCAG |
| | | CAAGATCATGCCTGCTATTGTCTTCCCAATCCTCCCCCTTGCTGTCCTGCCCC |
| | | ACCCCACCCCCCAGAATAGAATGACACCTACTCAGACAATGCGATGCAATT |
| | | TCCTCATTTTATTAGGAAAGGACAGTGGGAGTGGCACCTTCCAGGGTCAAG |
| | | GAAGGCACGGGGGAGGGGCAAACAACAGATGGCTGGCAACTAGAAGGCAC |
| | | AGTCGCATATGTCAGGCACCGGGCTTGCGGGTCATGCACCAGGTGCGCGGT |
| | | CCTTCGGGCACCTCGACGTCGGCGGTGACGGTGAAGCCGAGCCGCTCGTAG |
| | | AAGGGGAGGTTGCGGGGCGCGGATGTCTCCAGGAAGGCGGGCACCCCGGC |
| | | GCGCTCGGCCGCCTCCACTCCGGGGAGCACGACGGCGCTGCCCAGACCCTT |
| | | GCCCTGGTGGTCGGGCGAGACGCCGACGGTGGCCAGGAACCACGCGGGCTC |
| | | CTTGGGCCGGTGCGGCGCCAGGAGGCCTTCCATCTGTTGCTGCGCGGCCAG |
| | | CCGGGAACCGCTCAACTCGGCCATGCGCGGGCCGATCTCGGCGAACACCGC |
| | | CCCCGCTTCGACGCTCTCCGGCGTGGTCCAGACCGCCACCGCGGCGCCGTC |
| | | GTCCGCGACCCACACCTTGCCGATGTCGAGCCCGACGCGCGTGAGGAAGAG |
| | | TTCTTGCAGCTCGGTGACCCGCTCGATGTGGCGGTCCGGATCGACGGTGTGG |
| | | CGCGTGGCGGGGTAGTCGGCGAACGCGGCGGCGAGGGTGCGTACGGCCCT |
| | | GGGGACGTCGTCGCGGGTGGCGAGGCGCACCGTGGGCTTGTACTCGGTCAT |
| | | AGGACCGGGGTTTTCTTCCACGTCTCCTGCTTGCTTTAACAGAGAGAAGTTC |
| | | GTGGCACCGGATCCTCCAGCGCCTGTGCTATGTCTGCCCTCAGCTCTCTCAT |
| | | ATTGTTCCACGATGGTGTAGTCCCCATTATGGCTGATAATGTCGAGTTTAAT |
| | | ATCAGTCATGTAGGCGCCAGGCAGCTGCACTTGTTTCTTGGCCTTGTAGGTT |
| | | GTTTTGACCTCGGCATCGTAGTGTCCTCCGTCTTTGAGTTTCAGTCTCATTTT |
| | | AATTTCGCCTTTCAGAGCGCCATCCTCAGGATACATTCTCTCGGTGGAGGCT |
| | | TCCCATCCCATTGTTTTTTTTTGCATGACAGGGCCATCGGAGGGGAAGTTGG |
| | | TTCCTCTGAGTTTCACCTTATAAATAAACTCTCCGTCCTGGAGGGTGCTATC |
| | | TTGTGTGACTGTCACCACGCCTCCGTCCTCGAAGTTCATGAATCTCTCCCAC |
| | | TTGAAGCCTTCAGGGAAGGAGAGCTTCAGATAGTCAGGGATGTCGGCAGGG |
| | | TGTTTAACATAGGCTTTGCTTCCGTACTGGAACTGAGGGCTCAGAATATCCC |
| | | AGGAGAAGGGGAGGGGTCCTCCTTTTGTGACCTTGAGCTTAGCGGTTTGTGT |
| | | GCCCTCGTAAGGCCGGCCTTCTCCTTCTCCCTCAATCTCGAACTCGTGTCCG |
| | | TTGACGCTTCCCTCCATCTTGACCTTAAATCTCATAAACTCTTTGATGACATC |
| | | CTCGGAGCTGGCCATGGTGGCGATCGATAGGTCGAAAGGCCCGGAGATGAG |
| | | GAAGAGGAGAACAGCGCGGCAGACGTGCGCTTTTGAAGCGTGCAGAATGC |
| | | CGGGCCTCCGGAGGACCTTCGGGCGCCCGCCCCGCCCCTGAGCCCGCCCCT |
| | | GAGCCCGCCCCCGGACCCACCCTTCCCAGCTGCTGAGCCCAGAAAGCGAAG |
| | | GAGCAAAGCTGCTATTGGCCGCTGCCCCAAAGGCCTACCCGCTTCCATTGCT |
| | | CAGCGGTGCTGTCCATCTGCACGAGACTAGTGAGACGTGCTACTTCCATTTG |
| | | TCACGTCCTGCACGACGCGAGCTGCGGGGGGGGGGGGAACTTCCTGACTAG |
| | | GGGAGGAGTAGAAGGTGGCGCGAAGGGGCCACCAAAGAACGGAGCCGGTT |
| | | GGCGCTACCGGTGGATGTGGAATGTGTGCGAGGCCAGAGGCCACTTGTGTA |
| | | GCGCCAAGTGCCAGCGGGGCTGCTAAAGCGCATGCTCCAGACTGCCTTGGG |
| | | AAAAGCGCCTCCCCTACCCGGTAGAATTATCTTTCTAGAAAGGATCTGCGAT |
| | | CGCTCCGGTGCCCGTCAGTGGGCAGAGCGCACATCGCCCACAGTCCCCGAG |
| | | AAGTTGGGGGGAGGGGTCGGCAATTGAACGGGTGCCTAGAGAAGGTGGCG |
| | | CGGGGTAAACTGGGAAAGTGATGTCGTGTACTGGCTCCGCCTTTTTCCCGAG |
| | | GGTGGGGGAGAACCGTATATAAGTGCAGTAGTCGCCGTGAACGTTCTTTTT |
| | | CGCAACGGGTTTGCCGCCAGAACACAGCTGAAGCTTCGAGGGGCTCGCATC |
| | | TCTCCTTCACGCGCCCGCCGCCCTACCTGAGGCCGCCATCCACGCCGGTTGA |
| | | GTCGCGTTCTGCCGCCTCCCGCCTGTGGTGCCTCCTGAACTGCGTCCGCCGT |
| | | CTAGGTAAGTTTAAAGCTCAGGTCGAGACCGGGCCTTTGTCCGGCGCTCCCT |
| | | TGGAGCCTACCTAGACTCAGCCGGCTCTCCACGCTTTGCCTGACCCTGCTTG |
| | | CTCAACTCTACGTCTTTGTTTCGTTTTCTGTTCTGCGCCGTTACAGATCCAAG |
| | | CTGTGACCGGCGCCTACGAATTCGATATCGGTACCGCTAGCCCTAGGACGC |
| | | GTCAGACATGATAAGATACATTGATGAGTTTGGACAAACCACAACTAGAAT |
| | | GCAGTGAAAAAAATGCTTTATTTGTGAAATTTGTGATGCTATTGCTTTATTT |
| | | GTAACCATTATAAGCTGCAATAAACAAGTTAACAACAACAATTGCATTCAT |
| | | TTTATGTTTCAGGTTCAGGGGGAGGTGTGGGAGGTTTTTTATCGTTCTTCTTT |
| | | TATTCTCTCAAGATTTTCAGGCTGTATATTAAAACTTATATTAAGAACTATG |
| | | CTAACCACCTCATCAGGAACCGTTGTAGGTGGCGTGGGTTTTCTTGGCAATC |
| | | GACTCTCATGAAAACTACGAGCTAAATATTCAATATGTTCCTCTTGACCAAC |
| | | TTTATTCTGCATTTTTTTTGAACGAGGTTTAGAGCAAGCTTGTCGACGATGT |
| | | AGGTCACGGTCTCGAAGCCGCGGTGCGGGTGCCAGGGCGTGCCCTTGGGCT |
| | | CCCCGGGCGCGTACTCCACCTCACCCATCTGGTCCATCATGATGAACGGGTC |
| | | GAGGTGGCGGTAGTTGATCCCGGCGAACGCGCGGCGCACCGGGAAGCCCTC |
| | | GCCCTCGAAACCGCTGGGCGCGGTGGTCACGGTGAGCACGGGACGTGCGAC |
| | | GGCGTCGGCGGGTGCGGATACGCGGGGCAGCGTCAGCGGGTTCTCGACGGT |
| | | CACGGCGGGCATGTCGACAAGCTTCAGGAAACTGAGACAGGAATTTTATTA |
| | | AAAATTTAAATTTTGAAGAAAGTTCAGGGTTAATAGCATCCATTTTTTGCTT |
| | | TGCAAGTTCCTCAGCATTCTTAACAAAAGACGTCTCTTTTGACATGTTTAAA |
| | | GTTTAAACCTCCTGTGTGAAATTATTATCCGCTCATAATTCCACACATTATA |
| | | CGAGCCGGAAGCATAAAGTGTAAAGCCTGGGGTGCCTAATGAGTGAGCTAA |
| | | CTCACATTAATTGCGTTGCGCTCACTGCCAATTGCTTTCCAGTCGGGAAACC |
| | | TGTCGTGCCAGCTGCATTAATGAATCGGCCAACGCGCGGGGAGAGGCGGTT |
| | | TGCGTATTGGGCGCTCTTCCGCTTCCTCGCTCACTGACTCGCTGCGCTCGGT |
| | | CGTTCGGCTGCGGCGAGCGGTATCAGCTCACTCAAAGGCGGTAATACGGTT |
| | | ATCCACAGAATCAGGGGATAACGCAGGAAAGAACATGTGAGCAAAAGGCC |
| | | AGCAAAAGGCCAGGAACCGTAAAAAGGCCGCGTTGCTGGCGTTTTTCCATA |
| | | GGCTCCGCCCCCCTGACGAGCATCACAAAAATCGACGCTCAAGTCAGAGGT |
| | | GGCGAAACCCGACAGGACTATAAAGATACCAGGCGTTTCCCCCTGGAAGCT |
| | | CCCTCGTGCGCTCTCCTGTTCCGACCCTGCCGCTTACCGGATACCTGTCCGC |
| | | CTTTCTCCCTTCGGGAAGCGTGGCGCTTTCTCATAGCTCACGCTGTAGGTAT |
| | | CTCAGTTCGGTGTAGGTCGTTCGCTCCAAGCTGGGCTGTGTGCACGAACCCC |
| | | CCGTTCAGCCCGACCGCTGCGCCTTATCCGGTAACTATCGTCTTGAGTCCAA |
| | | CCCGGTAAGACACGACTTATCGCCACTGGCAGCAGCCACTGGTAACAGGAT |
| | | TAGCAGAGCGAGGTATGTAGGCGGTGCTACAGAGTTCTTGAAGTGGTGGCC |
| | | TAACTACGGCTACACTAGAAGGACAGTATTTGGTATCTGCGCTCTGCTGAA |
| | | GCCAGTTACCTTCGGAAAAAGAGTTGGTAGCTCTTGATCCGGCAAACAAAC |
| | | CACCGCTGGTAGCGGTGGTTTTTTTGTTTGCAAGCAGCAGATTACGCGCAGA |
| | | AAAAAAGGATCTCAAGAAGATCCTTTGATCTTTTCTACGGGGTCTGACGCTC |
| | | AGTGGAACGAAAACTCACGTTAAGGGATTTTGGTCATGAGATTATCAAAAA |
| | | GGATCTTCACCTAGATCCTTTTAAATTAAAAATGAAGTTTTAAATCAATCTA |
| | | AAGTATATATGAGTAAACTTGGTCTGACAGTTACCAATGCTTAATCAGTGA |
| | | GGCACCTATCTCAGCGATCTGTCTATTTCGTTCATCCATAGTTGCCTGACTCC |
| | | CCGTCGTGTAGATAACTACGATACGGGAGGGCTTACCATCTGGCCCCAGTG |
| | | CTGCAATGATACCGCGAGACCCACGCTCACCGGCTCCAGATTTATCAGCAA |
| | | TAAACCAGCCAGCCGGAAGGGCCGAGCGCAGAAGTGGTCCTGCAACTTTAT |
| | | CCGCCTCCATCCAGTCTATTAATTGTTGCCGGGAAGCTAGAGTAAGTAGTTC |
| | | GCCAGTTAATAGTTTGCGCAACGTTGTTGCCATTGCTACAGGCATCGTGGTG |
| | | TCACGCTCGTCGTTTGGTATGGCTTCATTCAGCTCCGGTTCCCAACGATCAA |
| | | GGCGAGTTACATGATCCCCCATGTTGTGCAAAAAAGCGGTTAGCTCCTTCG |
| | | GTCCTCCGATCGTTGTCAGAAGTAAGTTGGCCGCAGTGTTATCACTCATGGT |
| | | TATGGCAGCACTGCATAATTCTCTTACTGTCATGCCATCCGTAAGATGCTTT |
| | | TCTGTGACTGGTGAGTACTCAACCAAGTCATTCTGAGAATAGTGTATGCGGC |
| | | GACCGAGTTGCTCTTGCCCGGCGTCAATACGGGATAATACCGCGCCACATA |
| | | GCAGAACTTTAAAAGTGCTCATCATTGGAAAACGTTCTTCGGGGCGAAAAC |
| | | TCTCAAGGATCTTACCGCTGTTGAGATCCAGTTCGATGTAACCCACTCGTGC |
| | | ACCCAACTGATCTTCAGCATCTTTTACTTTCACCAGCGTTTCTGGGTGAGCA |
| | | AAAACAGGAAGGCAAAATGCCGCAAAAAAGGGAATAAGGGCGACACGGA |
| | | AATGTTGAATACTCATACTCTTCCTTTTTCAATATTATTGAAGCATTTATCAG |
| | | GGTTATTGTCTCATGAGCGGATACATATTTGAATGTATTTAGAAAAATAAAC |
| | | AAATAGGGGTTCCGCGCACATTTCCCCGAAAAGTGCCACCTGACGTCTAAG |
| | | AAACCATTATTATCATGACATTAACCTATAAAAATAGGCGTATCACGAGGC |
| | | C |
| 122 | FC550A- | GCCCCTGCAGCCGAATTATATTATTTTTGCCAAATAATTTTTAACAAAAGCT |
| | eBFP2 empty | CTGAAGTCTTCTTCATTTAAATTCTTAGATGATACTTCATCTGGAAAATTGTC |
| | vector | CCAATTAGTAGCATCACGCTGTGAGTAAGTTCTAAACCATTTTTTTATTGTT |
| | (EVeBFP2) | GTATTATCTCTAATCTTACTACTCGATGAGTTTTCGGTATTATCTCTATTTTT |
| | | AACTTGGAGCAGGTTCCATTCATTGTTTTTTTCATCATAGTGAATAAAATCA |
| | | ACTGCTTTAACACTTGTGCCTGAACACCATATCCATCCGGCGTAATACGACT |
| | | CACTATAGGGAGAGCGGCCGCCAGATCTTCCGGATGGCTCGAGTTTTTCAG |
| | | CAAGATCATGCCTGCTATTGTCTTCCCAATCCTCCCCCTTGCTGTCCTGCCCC |
| | | ACCCCACCCCCCAGAATAGAATGACACCTACTCAGACAATGCGATGCAATT |
| | | TCCTCATTTTATTAGGAAAGGACAGTGGGAGTGGCACCTTCCAGGGTCAAG |
| | | GAAGGCACGGGGGAGGGGCAAACAACAGATGGCTGGCAACTAGAAGGCAC |
| | | AGTCGCATATGTCAGGCACCGGGCTTGCGGGTCATGCACCAGGTGCGCGGT |
| | | CCTTCGGGCACCTCGACGTCGGCGGTGACGGTGAAGCCGAGCCGCTCGTAG |
| | | AAGGGGAGGTTGCGGGGCGCGGATGTCTCCAGGAAGGCGGGCACCCCGGC |
| | | GCGCTCGGCCGCCTCCACTCCGGGGAGCACGACGGCGCTGCCCAGACCCTT |
| | | GCCCTGGTGGTCGGGCGAGACGCCGACGGTGGCCAGGAACCACGCGGGCTC |
| | | CTTGGGCCGGTGCGGCGCCAGGAGGCCTTCCATCTGTTGCTGCGCGGCCAG |
| | | CCGGGAACCGCTCAACTCGGCCATGCGCGGGCCGATCTCGGCGAACACCGC |
| | | CCCCGCTTCGACGCTCTCCGGCGTGGTCCAGACCGCCACCGCGGCGCCGTC |
| | | GTCCGCGACCCACACCTTGCCGATGTCGAGCCCGACGCGCGTGAGGAAGAG |
| | | TTCTTGCAGCTCGGTGACCCGCTCGATGTGGCGGTCCGGATCGACGGTGTGG |
| | | CGCGTGGCGGGGTAGTCGGCGAACGCGGCGGCGAGGGTGCGTACGGCCCT |
| | | GGGGACGTCGTCGCGGGTGGCGAGGCGCACCGTGGGCTTGTACTCGGTCAT |
| | | AGGACCGGGGTTTTCTTCCACGTCTCCTGCTTGCTTTAACAGAGAGAAGTTC |
| | | GTGGCACCGGATCCCTTGTACAGCTCGTCCATGCCGAGAGTGATCCCGGCG |
| | | GCGGTGCGGAACTCCAGCAGGACCATGTGATCGCGCTTCTCGTTGGGGTCTT |
| | | TGCTCAGCACGGACTGGGTGCTCAGGTAGTGGCTGTCGGGCAGCAGCACGG |
| | | GGCCGTCGCCGATGGGGGTGTTCTGCTGGTAGTGGTCGGCGAGCTGCACGC |
| | | TGCCGTCCTCCACGTTGTGGCGGATCTTGAAGTTCACCTTGATGCCGTTCTT |
| | | CTGCTTGACGGCCATGATATAGATGTTGTGGCTGTTGAAGTTGTACTCCAGC |
| | | TTGTGCCCCAGGATGTTGCCGTCCTCCTTGAAGTCGACGCCCTTCAGCTCGA |
| | | TGCGGTTCACTAGGGTGTCGCCCTCGAACTTCACCTCGGCGCGGGTCTTGTA |
| | | GGTGCCGTCGTCCTTGAAGAAGATGGTGCGCTCCTGGACGTAGCCTTCGGG |
| | | CATGGCGGACTTGAAGAAGTCGTGCTGCTTCATGTGGTCGGGGTAGCGGGC |
| | | GAAGCACTGCACGCCGTGGCTCAGGGTGGTCACGAGGGTGGGCCAGGGCA |
| | | CGGGCAGCTTGCCGGTGGTGCAGATGAACTTCAGGGTCAGCTTGCCGTTGG |
| | | TGGCATCGCCCTCGCCCTCGCCCCTCACGCTGAACTTGTGGCCGTTTACGTC |
| | | GCCGTCCAGCTCGACCAGGATGGGCACCACCCCGGTGAACAGCTCCTCGCC |
| | | CTTGCTCACCATGGTGGCGATCGATAGGTCGAAAGGCCCGGAGATGAGGAA |
| | | GAGGAGAACAGCGCGGCAGACGTGCGCTTTTGAAGCGTGCAGAATGCCGG |
| | | GCCTCCGGAGGACCTTCGGGCGCCCGCCCCGCCCCTGAGCCCGCCCCTGAG |
| | | CCCGCCCCCGGACCCACCCTTCCCAGCTGCTGAGCCCAGAAAGCGAAGGAG |
| | | CAAAGCTGCTATTGGCCGCTGCCCCAAAGGCCTACCCGCTTCCATTGCTCAG |
| | | CGGTGCTGTCCATCTGCACGAGACTAGTGAGACGTGCTACTTCCATTTGTCA |
| | | CGTCCTGCACGACGCGAGCTGCGGGGGGGGGGGGAACTTCCTGACTAGGGG |
| | | AGGAGTAGAAGGTGGCGCGAAGGGGCCACCAAAGAACGGAGCCGGTTGGC |
| | | GCTACCGGTGGATGTGGAATGTGTGCGAGGCCAGAGGCCACTTGTGTAGCG |
| | | CCAAGTGCCAGCGGGGCTGCTAAAGCGCATGCTCCAGACTGCCTTGGGAAA |
| | | AGCGCCTCCCCTACCCGGTAGAATTATCTTTCTAGAAAGGATCTGCGATCGC |
| | | TCCGGTGCCCGTCAGTGGGCAGAGCGCACATCGCCCACAGTCCCCGAGAAG |
| | | TTGGGGGGAGGGGTCGGCAATTGAACGGGTGCCTAGAGAAGGTGGCGCGG |
| | | GGTAAACTGGGAAAGTGATGTCGTGTACTGGCTCCGCCTTTTTCCCGAGGGT |
| | | GGGGGAGAACCGTATATAAGTGCAGTAGTCGCCGTGAACGTTCTTTTTCGC |
| | | AACGGGTTTGCCGCCAGAACACAGCTGAAGCTTCGAGGGGCTCGCATCTCT |
| | | CCTTCACGCGCCCGCCGCCCTACCTGAGGCCGCCATCCACGCCGGTTGAGTC |
| | | GCGTTCTGCCGCCTCCCGCCTGTGGTGCCTCCTGAACTGCGTCCGCCGTCTA |
| | | GGTAAGTTTAAAGCTCAGGTCGAGACCGGGCCTTTGTCCGGCGCTCCCTTGG |
| | | AGCCTACCTAGACTCAGCCGGCTCTCCACGCTTTGCCTGACCCTGCTTGCTC |
| | | AACTCTACGTCTTTGTTTCGTTTTCTGTTCTGCGCCGTTACAGATCCAAGCTG |
| | | TGACCGGCGCCTACGAATTCGATATCGGTACCGCTAGCCCTAGGACGCGTC |
| | | AGACATGATAAGATACATTGATGAGTTTGGACAAACCACAACTAGAATGCA |
| | | GTGAAAAAAATGCTTTATTTGTGAAATTTGTGATGCTATTGCTTTATTTGTA |
| | | ACCATTATAAGCTGCAATAAACAAGTTAACAACAACAATTGCATTCATTTTA |
| | | TGTTTCAGGTTCAGGGGGAGGTGTGGGAGGTTTTTTATCGTTCTTCTTTTATT |
| | | CTCTCAAGATTTTCAGGCTGTATATTAAAACTTATATTAAGAACTATGCTAA |
| | | CCACCTCATCAGGAACCGTTGTAGGTGGCGTGGGTTTTCTTGGCAATCGACT |
| | | CTCATGAAAACTACGAGCTAAATATTCAATATGTTCCTCTTGACCAACTTTA |
| | | TTCTGCATTTTTTTTGAACGAGGTTTAGAGCAAGCTTGTCGACGATGTAGGT |
| | | CACGGTCTCGAAGCCGCGGTGCGGGTGCCAGGGCGTGCCCTTGGGCTCCCC |
| | | GGGCGCGTACTCCACCTCACCCATCTGGTCCATCATGATGAACGGGTCGAG |
| | | GTGGCGGTAGTTGATCCCGGCGAACGCGCGGCGCACCGGGAAGCCCTCGCC |
| | | CTCGAAACCGCTGGGCGCGGTGGTCACGGTGAGCACGGGACGTGCGACGGC |
| | | GTCGGCGGGTGCGGATACGCGGGGCAGCGTCAGCGGGTTCTCGACGGTCAC |
| | | GGCGGGCATGTCGACAAGCTTCAGGAAACTGAGACAGGAATTTTATTAAAA |
| | | ATTTAAATTTTGAAGAAAGTTCAGGGTTAATAGCATCCATTTTTTGCTTTGC |
| | | AAGTTCCTCAGCATTCTTAACAAAAGACGTCTCTTTTGACATGTTTAAAGTT |
| | | TAAACCTCCTGTGTGAAATTATTATCCGCTCATAATTCCACACATTATACGA |
| | | GCCGGAAGCATAAAGTGTAAAGCCTGGGGTGCCTAATGAGTGAGCTAACTC |
| | | ACATTAATTGCGTTGCGCTCACTGCCAATTGCTTTCCAGTCGGGAAACCTGT |
| | | CGTGCCAGCTGCATTAATGAATCGGCCAACGCGCGGGGAGAGGCGGTTTGC |
| | | GTATTGGGCGCTCTTCCGCTTCCTCGCTCACTGACTCGCTGCGCTCGGTCGTT |
| | | CGGCTGCGGCGAGCGGTATCAGCTCACTCAAAGGCGGTAATACGGTTATCC |
| | | ACAGAATCAGGGGATAACGCAGGAAAGAACATGTGAGCAAAAGGCCAGCA |
| | | AAAGGCCAGGAACCGTAAAAAGGCCGCGTTGCTGGCGTTTTTCCATAGGCT |
| | | CCGCCCCCCTGACGAGCATCACAAAAATCGACGCTCAAGTCAGAGGTGGCG |
| | | AAACCCGACAGGACTATAAAGATACCAGGCGTTTCCCCCTGGAAGCTCCCT |
| | | CGTGCGCTCTCCTGTTCCGACCCTGCCGCTTACCGGATACCTGTCCGCCTTTC |
| | | TCCCTTCGGGAAGCGTGGCGCTTTCTCATAGCTCACGCTGTAGGTATCTCAG |
| | | TTCGGTGTAGGTCGTTCGCTCCAAGCTGGGCTGTGTGCACGAACCCCCCGTT |
| | | CAGCCCGACCGCTGCGCCTTATCCGGTAACTATCGTCTTGAGTCCAACCCGG |
| | | TAAGACACGACTTATCGCCACTGGCAGCAGCCACTGGTAACAGGATTAGCA |
| | | GAGCGAGGTATGTAGGCGGTGCTACAGAGTTCTTGAAGTGGTGGCCTAACT |
| | | ACGGCTACACTAGAAGGACAGTATTTGGTATCTGCGCTCTGCTGAAGCCAG |
| | | TTACCTTCGGAAAAAGAGTTGGTAGCTCTTGATCCGGCAAACAAACCACCG |
| | | CTGGTAGCGGTGGTTTTTTTGTTTGCAAGCAGCAGATTACGCGCAGAAAAA |
| | | AAGGATCTCAAGAAGATCCTTTGATCTTTTCTACGGGGTCTGACGCTCAGTG |
| | | GAACGAAAACTCACGTTAAGGGATTTTGGTCATGAGATTATCAAAAAGGAT |
| | | CTTCACCTAGATCCTTTTAAATTAAAAATGAAGTTTTAAATCAATCTAAAGT |
| | | ATATATGAGTAAACTTGGTCTGACAGTTACCAATGCTTAATCAGTGAGGCA |
| | | CCTATCTCAGCGATCTGTCTATTTCGTTCATCCATAGTTGCCTGACTCCCCGT |
| | | CGTGTAGATAACTACGATACGGGAGGGCTTACCATCTGGCCCCAGTGCTGC |
| | | AATGATACCGCGAGACCCACGCTCACCGGCTCCAGATTTATCAGCAATAAA |
| | | CCAGCCAGCCGGAAGGGCCGAGCGCAGAAGTGGTCCTGCAACTTTATCCGC |
| | | CTCCATCCAGTCTATTAATTGTTGCCGGGAAGCTAGAGTAAGTAGTTCGCCA |
| | | GTTAATAGTTTGCGCAACGTTGTTGCCATTGCTACAGGCATCGTGGTGTCAC |
| | | GCTCGTCGTTTGGTATGGCTTCATTCAGCTCCGGTTCCCAACGATCAAGGCG |
| | | AGTTACATGATCCCCCATGTTGTGCAAAAAAGCGGTTAGCTCCTTCGGTCCT |
| | | CCGATCGTTGTCAGAAGTAAGTTGGCCGCAGTGTTATCACTCATGGTTATGG |
| | | CAGCACTGCATAATTCTCTTACTGTCATGCCATCCGTAAGATGCTTTTCTGT |
| | | GACTGGTGAGTACTCAACCAAGTCATTCTGAGAATAGTGTATGCGGCGACC |
| | | GAGTTGCTCTTGCCCGGCGTCAATACGGGATAATACCGCGCCACATAGCAG |
| | | AACTTTAAAAGTGCTCATCATTGGAAAACGTTCTTCGGGGCGAAAACTCTC |
| | | AAGGATCTTACCGCTGTTGAGATCCAGTTCGATGTAACCCACTCGTGCACCC |
| | | AACTGATCTTCAGCATCTTTTACTTTCACCAGCGTTTCTGGGTGAGCAAAAA |
| | | CAGGAAGGCAAAATGCCGCAAAAAAGGGAATAAGGGCGACACGGAAATGT |
| | | TGAATACTCATACTCTTCCTTTTTCAATATTATTGAAGCATTTATCAGGGTTA |
| | | TTGTCTCATGAGCGGATACATATTTGAATGTATTTAGAAAAATAAACAAAT |
| | | AGGGGTTCCGCGCACATTTCCCCGAAAAGTGCCACCTGACGTCTAAGAAAC |
| | | CATTATTATCATGACATTAACCTATAAAAATAGGCGTATCACGAGGCC |
| 123 | Consensus | MAAMAAGSITTLPALPEDGGSGAFPPGHFKDPKRLYCKNGGFFLRIHPDGRVD |
| | FGF2 | GVREKSDPHIKLQLQAEERGVVSIKGVCANRYLAMKEDGRLLASKCATDECFF |
| | | FERLESNNYNTYRSRKYSSWYVALKRTGQYKLGPKTGPGQKAILFLPMSAKS* |
| 124 | human FGF2 | MAAGSITTLPALPEDGGSGAFPPGHFKDPKRLYCKNGGFFLRIHPDGRVDGVRE |
| | | KSDPHIKLQLQAEERGVVSIKGVCANRYLAMKEDGRLLASKCVTDECFFFERL |
| | | ESNNYNTYRSRKYTSWYVALKRTGQYKLGSKTGPGQKAILFLPMSAKS* |
| 125 | Consensus | GPETLCGAELVDALQFVCGDRGFYFSKPTGYGSSSRRLHHKGIVDECCFQSCDL |
| | IGF1 | RRLEMYCAPIKPPKSA |
| 126 | Bovine IGF1 | GPETLCGAELVDALQFVCGDRGFYFSKPTGYGSSSRRLHHKGIVDECCFQSCDL |
| | | RRLEMYCAPIKPPKSA |
| 127 | Porcine IGF1 | GPETLCGAELVDALQFVCGDRGFYFSKPTGYGSSSRRLHHKGIVDECCFQSCDL |
| | | RRLEMYCAPIKPPKSA |
| 128 | Human IGF | GPETLCGAELVDALQFVCGPRGFYFSKPTGYGSSIRRLHHKGIVDECCFQSCDL |
| | | RRLEMYCAPIKPTKAA |
| 129 | Consensus | MCPQPARLEMNRCWALFLSLCCYLRLVSAEGDPIPEELYEMLSDHSIRSFDDLQ |
| | PDGFb | RLLHGDSVEEDGAELDLNXTRSHSGGELESLSRGRRSLGSXTIAEPAVIAECKTR |
| | | TEVFEISRRLIDRTNANFLVWPPCVEVQRCSGCCNNRNVQCRPTQVQXRXVQV |
| | | XKIEIVRKKPIFKKATVTLEDHLACRCETVXAXRPVTRXPGSSQEQRXAXTPQT |
| | | RVTIRTVRVRRPPKGKHRKFKHTHDKTALKETLGA |
| 130 | Bovine | MNRCWALFLSLCCYLRLVSAEGDPIPEELYKMLSDHSIRSFDDLQRLLHGDSVD |
| | PDGFb | EDGAELDLNLTRSHSGGELESLSRGRRSLGSPTVAAEPAVIAECKTRTEVFEISR |
| | | RLIDRTNANFLVWPPCVEVQRCSGCCNNRNVQCRPTQVQDRKVQVKKIEIVRK |
| | | KKIFKKATVTLVDHLACRCETVVARAVTRTPGSSQEQRARTPQTRVTIRTVRVR |
| | | RPPKGKHRKFKHTHDKTALKETLGA |
| 131 | Human | MNRCWALFLSLCCYLRLVSAEGDPIPEELYEMLSDHSIRSFDDLQRLLHGDPGE |
| | PDGFb | EDGAELDLNMTRSHSGGELESLARGRRSLGSLTIAEPAMIAECKTRTEVFEISRR |
| | | LIDRTNANFLVWPPCVEVQRCSGCCNNRNVQCRPTQVQLRPVQVRKIEIVRKK |
| | | PIFKKATVTLEDHLACKCETVAAARPVTRSPGGSQEQRAKTPQTRVTIRTVRVR |
| | | RPPKGKHRKFKHTHDKTALKETLGA |

*Indicates a stop codon

### 10. EQUIVALENTS AND INCORPORATION BY REFERENCE

All references cited herein are incorporated by reference to the same extent as if each individual publication, database entry (e.g., Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it is understood by persons skilled in the relevant art that various changes in form and details can be made therein without departing from the spirit and scope of the invention

What is claimed is:

1. A method for culturing a bovine cell line in a cell culture media, said method comprising:
(a) seeding an edible cell culture media with an edible, bovine cell line arising from cells transduced with
a polynucleotide comprising a single coding sequence encoding a single growth factor receptor or growth factor, which is an FGFR, a PDGFR, an activated FGFR, or an activated PDGFR, or a combination of solely a coding sequence encoding an FGFR or an activated FGFR and a coding sequence encoding an IGF;
wherein the polynucleotide is engineered to express the coding sequence or the combination of coding sequences in the cell line; and
(b) culturing the cell line in the cell culture media substantially free of exogenous growth factors,
wherein the cell line proliferates in the absence of exogenous growth factors over 72 hours of culture to a cell density equivalent to a cell density over 72 hours of culture time of a comparable cell line not comprising the polynucleotide and cultured in the presence of the exogenous growth factors.

2. The method of claim 1, wherein the cells are transduced with only a polynucleotide encoding a single growth factor or growth factor receptor.

3. The method of claim 1, wherein the coding sequence encodes an FGFR or activated FGFR.

4. The method of claim 1, wherein the coding sequence encodes a PDGFR or activated PDGFR.

5. The method of claim 1, wherein the polynucleotide comprises the combination of a coding sequence encoding an FGFR or an activated FGFR and a coding sequence encoding IGF.

6. The method of claim 1, wherein the activated FGFR is FGFR3 having an N540K modification, a K650E modification, a myrist modification, or some combination thereof.

7. The method of claim 1, wherein the cell line is cultured in an absence of serum.

8. The method of claim 1, wherein the cell line is capable of proliferating to a cell density double the seeded cell density over 72 hours of culture time without exogenous growth factors.

9. The method of claim 1, wherein the cell line is cultured to a cell density of 1 million cells per milliliter or more.

10. The method of claim 1, wherein the coding sequence for the IGF encodes a fusion protein comprising the IGF and a signal peptide, wherein the signal peptide is IL2 signal peptide.

11. The method of claim 1, wherein the coding sequence encodes an FGFR and is one of FGFR1, FGFR2, FGFR3, or FGFR4.

12. The method of claim 1, wherein the cells of the cell line are myoblasts, myocytes, satellite cells, side population cells, muscle derived stem cells, mesenchymal stem cells, myogenic pericytes, mesoangioblasts, or fibroblasts.

13. The method of claim 1, further comprising the step of forming cell line into a cell-based food product suitable for consumption.

14. A method for culturing an edible, bovine cell line in a cell culture media, said method comprising:
(a) seeding an edible cell culture media with an edible, bovine cell line arising from cells transduced with
a polynucleotide comprising a combination of solely a coding sequence encoding IGF1 and a coding sequence encoding FGF;
wherein the polynucleotide is engineered to express the coding sequences in the cell line; and
(b) culturing the cell line in the cell culture media substantially free of exogenous growth factors,
wherein the cell line proliferates in the absence of the exogenous growth factors over 72 hours of culture to a cell density equivalent to a cell density over 72 hours of culture time of a comparable cell line not comprising the polynucleotide and cultured in the presence of exogenous IGF1 and FGF.

15. A method for culturing an edible, bovine cell line in a cell culture media, said method comprising:
(a) seeding an edible cell culture media with an edible, bovine cell line arising from cells transduced with
a polynucleotide comprising a single coding sequence of a single growth factor, which encodes FGF2 or FGF3;
wherein the polynucleotide is engineered to express the coding sequence in the cell line; and
(b) culturing the cell line in the cell culture media substantially free of exogenous growth factors, wherein the cell line is capable of proliferating to a cell density higher than the seeded cell density over 72 hours of culture time of a comparable cell line not comprising the polynucleotide.

16. The method of claim 14, wherein the cells of the cell line are myoblasts, myocytes, satellite cells, side population cells, muscle derived stem cells, mesenchymal stem cells, myogenic pericytes, mesoangioblasts, or fibroblasts.

17. The method of claim 14, further comprising the step of forming the cell line into a cell-based food product suitable for consumption.

18. The method of claim 15, wherein the cells of the cell line are myoblasts, myocytes, satellite cells, side population cells, muscle derived stem cells, mesenchymal stem cells, myogenic pericytes, mesoangioblasts, or fibroblasts.

19. The method of claim 15, further comprising the step of forming the cell line into a cell-based food product suitable for consumption.

20. The method of claim 15, wherein the cells are transduced with only a single type of a growth factor polynucleotide, wherein the growth factor polynucleotide encodes one or more growth factors.


SEQUENCE LISTING (131 sequences)

SEQ ID NO: 1 - protein, 158 aa, synthetic construct
MAAGAAGSITTLPALPDDGGGGAFPPGHFKDPKRLYCKNGGFFLRINPDGRVDGVREKSDPHIKLQLQAEERGVVSIKGVSANRFLAMKEDGRLLALKCATEECFFFERLESNNYNTYRSRKYSDWYVALKRTGQYKPGPKTGPGQKAILFLPMSAKS

SEQ ID NO: 2 - protein, 158 aa, synthetic construct
MAAGAAGSITTLPALPDDGGGGAFPPGHFKDPKRLYCKNGGFFLLINPDGRVDGTREKSDPFIKLQLQAEERGVVSIKGVSANRFLAMKEDGRLYALKYATEECFFFERLEENNYNTYRSRKYSDWYVALKRTGQYKPGPKTGPGQKAILFLPMSAKS

SEQ ID NO: 3 - protein, 175 aa, synthetic construct
MRAWIFFLLCLAGRALAMAAGAAGSITTLPALPDDGGGGAFPPGHFKDPKRLYCKNGGFFLRINPDGRVDGVREKSDPHIKLQLQAEERGVVSIKGVSANRFLAMKEDGRLLALKCATEECFFFERLESNNYNTYRSRKYSDWYVALKRTGQYKPGPKTGPGQKAILFLPMSAKS

SEQ ID NO: 4 - protein, 179 aa, synthetic construct
MMCKVLIFGCISVAMLMTTAYMAAGAAGSITTLPALPDDGGGGAFPPGHFKDPKRLYCKNGGFFLRINPDGRVDGVREKSDPHIKLQLQAEERGVVSIKGVSANRFLAMKEDGRLLALKCATEECFFFERLESNNYNTYRSRKYSDWYVALKRTGQYKPGPKTGPGQKAILFLPMSAKS

SEQ ID NO: 5 - protein, 175 aa, synthetic construct
MGVKVLFALICIAVAEAMAAGAAGSITTLPALPDDGGGGAFPPGHFKDPKRLYCKNGGFFLRINPDGRVDGVREKSDPHIKLQLQAEERGVVSIKGVSANRFLAMKEDGRLLALKCATEECFFFERLESNNYNTYRSRKYSDWYVALKRTGQYKPGPKTGPGQKAILFLPMSAKS

SEQ ID NO: 6 - protein, 178 aa, synthetic construct
MYRMQLLSCIALSLALVTNSMAAGAAGSITTLPALPDDGGGGAFPPGHFKDPKRLYCKNGGFFLRINPDGRVDGVREKSDPHIKLQLQAEERGVVSIKGVSANRFLAMKEDGRLLALKCATEECFFFERLESNNYNTYRSRKYSDWYVALKRTGQYKPGPKTGPGQKAILFLPMSAKS

SEQ ID NO: 7 - protein, 177 aa, synthetic construct
MRMQLLLLIALSLALVTNSMAAGAAGSITTLPALPDDGGGGAFPPGHFKDPKRLYCKNGGFFLRINPDGRVDGVREKSDPHIKLQLQAEERGVVSIKGVSANRFLAMKEDGRLLALKCATEECFFFERLESNNYNTYRSRKYSDWYVALKRTGQYKPGPKTGPGQKAILFLPMSAKS

SEQ ID NO: 8 - protein, 178 aa, synthetic construct
MRRMQLLLLIALSLALVTNSMAAGAAGSITTLPALPDDGGGGAFPPGHFKDPKRLYCKNGGFFLRINPDGRVDGVREKSDPHIKLQLQAEERGVVSIKGVSANRFLAMKEDGRLLALKCATEECFFFERLESNNYNTYRSRKYSDWYVALKRTGQYKPGPKTGPGQKAILFLPMSAKS

SEQ ID NO: 9 - protein, 179 aa, synthetic construct
MMCKVLIFGCISVAMLMTTAYMAAGAAGSITTLPALPDDGGGGAFPPGHFKDPKRLYCKNGGFFLLINPDGRVDGTREKSDPFIKLQLQAEERGVVSIKGVSANRFLAMKEDGRLYALKYATEECFFFERLEENNYNTYRSRKYSDWYVALKRTGQYKPGPKTGPGQKAILFLPMSAKS

SEQ ID NO: 10 - protein, 178 aa, synthetic construct
MYRMQLLSCIALSLALVTNSMAAGAAGSITTLPALPDDGGGGAFPPGHFKDPKRLYCKNGGFFLLINPDGRVDGTREKSDPFIKLQLQAEERGVVSIKGVSANRFLAMKEDGRLYALKYATEECFFFERLEENNYNTYRSRKYSDWYVALKRTGQYKPGPKTGPGQKAILFLPMSAKS

SEQ ID NO: 11 - protein, 208 aa, synthetic construct
MNFTEGCEATGRRPGSAGSRRRRAPRPGPVALLPLLLPLLLPPAAAVPLPMAAGAAGSITTLPALPDDGGGGAFPPGHFKDPKRLYCKNGGFFLRINPDGRVDGVREKSDPHIKLQLQAEERGVVSIKGVSANRFLAMKEDGRLLALKCATEECFFFERLESNNYNTYRSRKYSDWYVALKRTGQYKPGPKTGPGQKAILFLPMSAKS

SEQ ID NO: 12 - protein, 187 aa, synthetic construct
MNSFSTSAFGPVAFSLGLLLVLPAAFPAPMAAGAAGSITTLPALPDDGGGGAFPPGHFKDPKRLYCKNGGFFLRINPDGRVDGVREKSDPHIKLQLQAEERGVVSIKGVSANRFLAMKEDGRLLALKCATEECFFFERLESNNYNTYRSRKYSDWYVALKRTGQYKPGPKTGPGQKAILFLPMSAKS

SEQ ID NO: 13 - protein, 178 aa, synthetic construct; identical to SEQ ID NO: 10

SEQ ID NO: 14 - protein, 185 aa, synthetic construct
MALTFALLVALLVLSCKSSCSVGMVSKMAAGAAGSITTLPALPDDGGGGAFPPGHFKDPKRLYCKNGGFFLRINPDGRVDGVREKSDPHIKLQLQAEERGVVSIKGVSANRFLAMKEDGRLLALKCATEECFFFERLESNNYNTYRSRKYSDWYVALKRTGQYKPGPKTGPGQKAILFLPMSAKS

SEQ ID NO: 15 - protein, 179 aa, synthetic construct
MWWRLWWLLLLLLLLWPMVWAMAAGAAGSITTLPALPDDGGGGAFPPGHFKDPKRLYCKNGGFFLRINPDGRVDGVREKSDPHIKLQLQAEERGVVSIKGVSANRFLAMKEDGRLLALKCATEECFFFERLESNNYNTYRSRKYSDWYVALKRTGQYKPGPKTGPGQKAILFLPMSAKS

SEQ ID NO: 16 - protein, 153 aa, synthetic construct
MEKINSLSTQLVKCCFCDFLKVKMHTVSYIHFFYLGLCLLTLTSSAAAGPETLCGAELVDALQFVCGDRGFYFSKPTGYGSSSRRLHHKGIVDECCFQSCDLRRLEMYCAPIKPPKSARSVRAQRHTDMPKAQKEVHLKNTSRGNTGNRNYRM

SEQ ID NO: 17 - protein, 71 aa, synthetic construct
MGPETLCGAELVDALQFVCGDRGFYFSKPTGYGSSSRRLHHKGIVDECCFQSCDLRRLEMYCAPIKPPKSA

SEQ ID NO: 18 - protein, 251 aa, synthetic construct
MCPQPARLEPGMNFGVVFAVILSLPLARLEGDPIPEDIYEILGGSSVRSISDLQRALRIDSVEEDSSSLDLNATQPSQNHVSLSRERRSLDALAAAEPAVLAECKTRTVVFEISRDMVDSTNANFVVWPPCVEVQRCSGCCNNRNVQCRPMQIRVRHVQVNKIEFFQRKPIFKKVIVPLEDHVQCRCEVVSRPPPRSNRPASREQRRFSPSFTTAAISQRKRVRRPPAQKRKHKKYKHVNDKKVLKEILIA

SEQ ID NO: 19 - protein, 219 aa, synthetic construct
MCPQPARLEPGMNFGVVFAVILSLPLARLEGDPIPEDIYEILGGSSVRSISDLQRALRIDSVEEDSSSLDLNATQPSQNHVSLSRERRSLDALAAAEPAVLAECKTRTVVFEISRDMVDSTNANFVVWPPCVEVQRCSGCCNNRNVQCRPMQIRVRHVQVNKIEFFQRKPIFKKVIVPLEDHVQCRCEVVSRPPPRSNRPASREQRRFSPSFTTAAISQ

SEQ ID NO: 20 - protein, 45 aa, synthetic construct
MADYKDDDDKKGGIIVAILLLIVMLAIEILLLITLIIAVTSGGSG

SEQ ID NO: 21 - protein, 37 aa, synthetic construct
MAKGGIIVAILLLIVMLAIEILLLITLIIAVTSGGSG

SEQ ID NO: 22 - protein, 20 aa, synthetic construct
MYRMQLLSCIALSLALVTNS

SEQ ID NO: 23 - protein, 19 aa, synthetic construct
MRMQLLLLIALSLALVTNS

SEQ ID NO: 24 - protein, 20 aa, synthetic construct
MRRMQLLLLIALSLALVTNS

SEQ ID NO: 25 - protein, 21 aa, synthetic construct
MMCKVLIFGCISVAMLMTTAY

SEQ ID NO: 26 - protein, 27 aa, synthetic construct
MALTFALLVALLVLSCKSSCSVGMVSK

SEQ ID NO: 27 - protein, 29 aa, synthetic construct
MNSFSTSAFGPVAFSLGLLLVLPAAFPAP

SEQ ID NO: 28 - protein, 50 aa, synthetic construct
MNFTEGCEATGRRPGSAGSRRRRAPRPGPVALLPLLLPLLLPPAAAVPLP

SEQ ID NO: 29 - protein, 17 aa, synthetic construct
MGVKVLFALICIAVAEA

SEQ ID NO: 30 - protein, 21 aa, synthetic construct
MWWRLWWLLLLLLLLWPMVWA

SEQ ID NO: 31 - protein, 17 aa, synthetic construct
MRAWIFFLLCLAGRALA

SEQ ID NO: 32 - protein, 819 aa, synthetic construct
MFTWRCLILWAVLVTATLSAARPAPTLPDQALPKANIEVESHSAHPGDLLQLRCRLRDDVQSINWVRDGVQLPENNRTRITGEEVEVRDAVPEDSGLYACMTNSPSGSETTYFSVNVSDALPSAEDDDDEDDSSSEEKEADNTKPNQAVAPYWTYPEKMEKKLHAVPAAKTVKFKCPSGGTPNPTLRWLKNGKEFKPDHRIGGYKVRYATWSIIMDSVVPSDKGNYTCIVENKYGSINHTYQLDVVERSPHRPILQAGLPANKTVALGSNVEFVCKVYSDPQPHIQWLKHIEVNGSKIGPDNLPYVQILKTAGVNTTDKEMEVLHLRNVSFEDAGEYTCLAGNSIGISHHSAWLTVLEATEQSPAMMTSPLYLEIIIYCTGAFLISCMVVTVIIYKMKSTTKKTDFNSQLAVHKLAKSIPLRRQVTVSADSSSSMNSGVMLVRPSRLSSSGTPMLAGVSEYELPEDPRWELPRDRLILGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGMEYCYNPTRIPEEQLSFKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVAMTSNQEYLDLSVPLDQYSPGFPATRSSTCSSGEDSVFSHDPLPDEPCLPRCPPHSHGALKRH

SEQ ID NO: 33 - protein, 819 aa, synthetic construct
MFTWRCLILWAVLVTATLSAARPAPTLPDQALPKANIEVESHSAHPGDLLQLRCRLRDDVQSINWVRDGVQLPENNRTRITGEEVEVRDAVPEDSGLYACMTNSPSGSETTYFSVNVSDALPSAEDDDDEDDSSSEEKEADNTKPNQAVAPYWTYPEKMEKKLHAVPAAKTVKFKCPSGGTPNPTLRWLKNGKEFKPDHRIGGYKVRYATWSIIMDSVVPSDKGNYTCIVENKYGSINHTYQLDVVERSPHRPILQAGLPANKTVALGSNVEFVCKVYSDPQPHIQWLKHIEVNGSKIGPDNLPYVQILKTAGVNTTDKEMEVLHLRNVSFEDAGEYTCLAGNSIGISHHSAWLTVLEATEQSPAMMTSPLYLEIIIYCTGAFLISCMVVTVIIYKMKSTTKKTDFNSQLAVHKLAKSIPLRRQVTVSADSSSSMNSGVMLVRPSRLSSSGTPMLAGVSEYELPEDPRWELPRDRLILGKPLGEGCFGQVVLAEAIGLDKDKPNRVTKVAVKMLKSDATEKDLSDLISEMEMMKMIGKHKNIIKLLGACTQDGPLYVIVEYASKGNLREYLQARRPPGMEYCYNPTRIPEEQLSFKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVAMTSNQEYLDLSVPLDQYSPGFPATRSSTCSSGEDSVFSHDPLPDEPCLPRCPPHSHGALKRH

SEQ ID NO: 34 - protein, 819 aa, synthetic construct
MFTWRCLILWAVLVTATLSAARPAPTLPDQALPKANIEVESHSAHPGDLLQLRCRLRDDVQSINWVRDGVQLPENNRTRITGEEVEVRDAVPEDSGLYACMTNSPSGSETTYFSVNVSDALPSAEDDDDEDDSSSEEKEADNTKPNQAVAPYWTYPEKMEKKLHAVPAAKTVKFKCPSGGTPNPTLRWLKNGKEFKPDHRIGGYKVRYATWSIIMDSVVPSDKGNYTCIVENKYGSINHTYQLDVVERSPHRPILQAGLPANKTVALGSNVEFVCKVYSDPQPHIQWLKHIEVNGSKIGPDNLPYVQILKTAGVNTTDKEMEVLHLRNVSFEDAGEYTCLAGNSIGISHHSAWLTVLEATEQSPAMMTSPLYLEIIIYCTGAFLISCMVVTVIIYKMKSTTKKTDFNSQLAVHKLAKSIPLRRQVTVSADSSSSMNSGVMLVRPSRLSSSGTPMLAGVSEYELPEDPRWELPRDRLILGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGMEYCYNPTRIPEEQLSFKDLVSCAYQVARGMEYLASKKCIHRDLAARNVLVTEDNVMKIADFGLARDIHHIDYYKETTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVAMTSNQEYLDLSVPLDQYSPGFPATRSSTCSSGEDSVFSHDPLPDEPCLPRCPPHSHGALKRH

SEQ ID NO: 35 - protein, 819 aa, synthetic construct
MFTWRCLILWAVLVTATLSAARPAPTLPDQALPKANIEVESHSAHPGDLLQLRCRLRDDVQSINWVRDGVQLPENNRTRITGEEVEVRDAVPEDSGLYACMTNSPSGSETTYFSVNVSDALPSAEDDDDEDDSSSEEKEADNTKPNQAVAPYWTYPEKMEKKLHAVPAAKTVKFKCPSGGTPNPTLRWLKNGKEFKPDHRIGGYKVRYATWSIIMDSVVPSDKGNYTCIVENKYGSINHTYQLDVVERSPHRPILQAGLPANKTVALGSNVEFVCKVYSDPQPHIQWLKHIEVNGSKIGPDNLPYVQILKTAGVNTTDKEMEVLHLRNVSFEDAGEYTCLAGNSIGISHHSAWLTVLEATEQSPAMMTSPLYLEIIIYCTGAFLISCMVVTVIIYKMKSTTKKTDFNSQLAVHKLAKSIPLRRQVTVSADSSSSMNSGVMLVRPSRLSSSGTPMLAGVSEYELPEDPRWELPRDRLILGKPLGEGCFGQVVLAEAIGLDKDKPNRVTKVAVKMLKSDATEKDLSDLISEMEMMKMIGKHKNIIKLLGACTQDGPLYVIVEYASKGNLREYLQARRPPGMEYCYNPTRIPEEQLSFKDLVSCAYQVARGMEYLASKKCIHRDLAARNVLVTEDNVMKIADFGLARDIHHIDYYKETTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVAMTSNQEYLDLSVPLDQYAPGFPATRSSTCSSGEDSVFSHDPLPDEPCLPRCPPHSHGALKRH

SEQ ID NO: 36 - protein, 438 aa, synthetic construct
MGSSKSKPKDPSQRKMKSTTKKTDFNSQLAVHKLAKSIPLRRQVTVSADSSSSMNSGVMLVRPSRLSSSGTPMLAGVSEYELPEDPRWELPRDRLILGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGMEYCYNPTRIPEEQLSFKDLVSCAYQVARGMEYLASKKCIHRDLAARNVLVTEDNVMKIADFGLARDIHHIDYYKETTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVAMTSNQEYLDLSVPLDQYSPGFPATRSSTCSSGEDSVFSHDPLPDEPCLPRCPPHSHGALKRH

SEQ ID NO: 37 - protein, 840 aa, synthetic construct
MGLKSTWRYGNGPGTYSKKMVSWDSGCLICLVVVTMAGLSLARPSFNLVVEDATLEPEEPPTKYQISQPDVHSALPGEPLELRCQLKDAVMISWTKDGVPLGPDNRTVIIGEYLQIKDASPRDSGLYACTAIRTLDSDTLYFIVNVTDALSSGDDEDDNDGSEDFVNDSNQMRAPYWTHTDKMEKRLHAVPAANTVKFRCPAMGNPTPTMRWLKNGKEFKQEHRIGGYKVRNQHWSLIMESVVPSDKGNYTCIVENQYGSINHTYHLDVVERSPHRPILQAGLPANASAVVGGDVEFVCKVYSDAQPHIQWIKHVERNGSKYGPDGLPYLQVLKAAGVNTTDKEIEVLYIRNVTFEDAGEYTCLAGNSIGISFHTAWLTVLPAPEKEKEFPTSPDYLEIAIYCIGVFLIACMVLTVILCRMKNTTKKPDFSSQPAVHKLTKRIPLRRQVSADSSSSMNSNTPLVRITTRLSSTADAPMLAGVSEYELPEDPKWEFPRDKLTLGKPLGEGCFGQVVMAEAVGIDKDRPKEAVTVAVKMLKDDATEKDLSDLVSEMEMMKMIGKHKNIINLLGACTQDGPLYVIVEYASKGNLREYLRARRPPGMEYSFDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWQAVPSQRPTFKQLVEDLDRILTLTTNEEYLDLSGPLEQYSPSYPDTRSSCSSGDDSVFSPDPMPYEPCLPKYQHMNGSVKT

SEQ ID NO: 38 - protein, 840 aa, synthetic construct
MGLKSTWRYGNGPGTYSKKMVSWDSGCLICLVVVTMAGLSLARPSFNLVVEDATLEPEEPPTKYQISQPDVHSALPGEPLELRCQLKDAVMISWTKDGVPLGPDNRTVIIGEYLQIKDASPRDSGLYACTAIRTLDSDTLYFIVNVTDALSSGDDEDDNDGSEDFVNDSNQMRAPYWTHTDKMEKRLHAVPAANTVKFRCPAMGNPTPTMRWLKNGKEFKQEHRIGGYKVRNQHWSLIMESVVPSDKGNYTCIVENQYGSINHTYHLDVVERSPHRPILQAGLPANASAVVGGDVEFVCKVYSDAQPHIQWIKHVERNGSKYGPDGLPYLQVLKAAGVNTTDKEIEVLYIRNVTFEDAGEYTCLAGNSIGISFHTAWLTVLPAPEKEKEFPTSPDYLEIAIYCIGVFLIACMVLTVILCRMKNTTKKPDFSSQPAVHKLTKRIPLRRQVSADSSSSMNSNTPLVRITTRLSSTADAPMLAGVSEYELPEDPKWEFPRDKLTLGKPLGEGCFGQVVMAEAVGIDKDRPKEAVTVAVKMLKDDATEKDLSDLVSEMEMMKMIGKHKNIIKLLGACTQDGPLYVIVEYASKGNLREYLRARRPPGMEYSFDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWQAVPSQRPTFKQLVEDLDRILTLTTNEEYLDLSGPLEQYSPSYPDTRSSCSSGDDSVFSPDPMPYEPCLPKYQHMNGSVKT

SEQ ID NO: 39 - protein, 840 aa, synthetic construct
MGLKSTWRYGNGPGTYSKKMVSWDSGCLICLVVVTMAGLSLARPSFNLVVEDATLEPEEPPTKYQISQPDVHSALPGEPLELRCQLKDAVMISWTKDGVPLGPDNRTVIIGEYLQIKDASPRDSGLYACTAIRTLDSDTLYFIVNVTDALSSGDDEDDNDGSEDFVNDSNQMRAPYWTHTDKMEKRLHAVPAANTVKFRCPAMGNPTPTMRWLKNGKEFKQEHRIGGYKVRNQHWSLIMESVVPSDKGNYTCIVENQYGSINHTYHLDVVERSPHRPILQAGLPANASAVVGGDVEFVCKVYSDAQPHIQWIKHVERNGSKYGPDGLPYLQVLKAAGVNTTDKEIEVLYIRNVTFEDAGEYTCLAGNSIGISFHTAWLTVLPAPEKEKEFPTSPDYLEIAIYCIGVFLIACMVLTVILCRMKNTTKKPDFSSQPAVHKLTKRIPLRRQVSADSSSSMNSNTPLVRITTRLSSTADAPMLAGVSEYELPEDPKWEFPRDKLTLGKPLGEGCFGQVVMAEAVGIDKDRPKEAVTVAVKMLKDDATEKDLSDLVSEMEMMKMIGKHKNIINLLGACTQDGPLYVIVEYASKGNLREYLRARRPPGMEYSFDINRVPEEQMTFKDLVSCTYQLARGMEYLASQKCIHRDLAARNVLVTENNVMKIADFGLARDINNIDYYKETTNGRLPVKWMAPEALFDRVYTHQSDVWSFGVLMWEIFTLGGSPYPGIPVEELFKLLKEGHRMDKPANCTNELYMMMRDCWQAVPSQRPTFKQLVEDLDRILTLTTNEEYLDLSGPLEQYSPSYPDTRSSCSSGDDSVFSPDPMPYEPCLPKYQHMNGSVKT

SEQ ID NO: 40 - protein, 840 aa, synthetic construct
MGLKSTWRYGNGPGTYSKKMVSWDSGCLICLVVVTMAGLSLARPSFNLVVEDATLEPEEPPTKYQISQPDVHSALPGEPLELRCQLKDAVMISWTKDGVPLGPDNRTVIIGEYLQIKDASPRDSGLYACTAIRTLDSDTLYFIVNVTDALSSGDDEDDNDGSEDFVNDSNQMRAPYWTHTDKMEKRLHAVPAANTVKFRCPAMGNPTPTMRWLKNGKEFKQEHRIGGYKVRNQHWSLIMESVVPSDKGNYTCIVENQYGSINHTYHLDVVERSPHRPILQAGLPANASAVVGGDVEFVCKVYSDAQPHIQWIKHVERNGSKYGPDGLPYLQVLKAAGVNTTDKEIEVLYIRNVTFEDAGEYTCLAGNSIGISFHTAWLTVLPAPEKEKEFPTSPDYLEIAIYCIGVFLIACMVLTVILCRMKNTTKKPDFSSQPAVHKLTKRIPLRRQVSADSSSSMNSNTPLVRITTRLSSTADAPMLAGVSEYELPEDPKWEFPRDKLTLGKPLGEGCFGQVVMAEAVGIDKDRPKEAVTVAVKMLKDDATEKDLSDLVSEMEMMKMIGKHKNIIKLLGACTQDGPLYVIVEYASKGNLREYLRARRPPGMEYSFDINRVPEEQMTFKDLVSCTYQLARGMEYLASQKCIHRDLAARNVLVTENNVMKIADFGLARDINNIDYYKETTNGRLPVKWMAPEALFDRVYTHQSDVWSFGVLMWEIFTLGGSPYPGIPVEELFKLLKEGHRMDKPANCTNELYMMMRDCWQAVPSQRPTFKQLVEDLDRILTLTTNEEYLDLSGPLEQYAPSYPDTRSSCSSGDDSVFSPDPMPYEPCLPKYQHMNGSVKT

SEQ ID NO: 41 - protein, 812 aa, synthetic construct
MSEAGGGAAAAASLPRSRAGGMRAAWGSVWCLCLAAAVGALPAARRRGAERSGGQAAEYLRSETAFLEELVFGSGDTIELSCNTQSSSVSVFWFKDGIGIAPSNRTHIGQKLLKIINVSYDDSGLYSCKPRHSNEVLGNFTVRVTGVPFWTRPDKMEKKLLAVPAANTVRFRCPAGGNPTPTIYWLKNGKEFKGEHRIGGIKLRHQQWSLVMESVVPSDRGNYTCVVENKYGNIRHTYQLDVLERSPHRPILQAGLPANQTVVVGSNVEFHCKVYSDAQPHIQWLKHVEVNGSKYGPDGTPYVTVLKSWISKNAEADANLNLFNVTEQDEGEYLCRANNFVGIAEKPFWLHIRKPKPAEELMEMDDSGSVYAGILSYGTGLVLFILVLVIVIICRMKMPNKKAMNTTTVQKVSKFPLKRQVTVSLESNSSMNSNTPLVRITRLSSSDGPMLANVSELELPPDPKWELARSRLTLGKPLGEGCFGQVVMAEAIGIDKDKPNKAITVAVKMLKDDATDKDLSDLVSEMEMMKMIGKHKNIINLLGACTQDGPLYVLVEYASKGNLREYLRARRPPGMDYSFDTCKLPEEQLTFKDLVSCAYQVARGMEYLASQKCIHRDLAARNVLVTEDNVMKIADFGLARDVHNIDYYKKTTNGRLPVKWMAPEALFDRVYTHQSDVWSFGVLLWEIFTLGGSPYPGIPVEELFKLLKEGHRMDKPANCTHDLYMIMRECWHAVPSQRPTFKQLVEDLDRVLTMTSTDEYLDLSVPFEQYSPAGQDTHSTCSSGDDSVFAHDLLPDEPCLPKHVPCNGVIRT

SEQ ID NO: 42 - protein, 812 aa, synthetic construct
MSEAGGGAAAAASLPRSRAGGMRAAWGSVWCLCLAAAVGALPAARRRGAERSGGQAAEYLRSETAFLEELVFGSGDTIELSCNTQSSSVSVFWFKDGIGIAPSNRTHIGQKLLKIINVSYDDSGLYSCKPRHSNEVLGNFTVRVTGVPFWTRPDKMEKKLLAVPAANTVRFRCPAGGNPTPTIYWLKNGKEFKGEHRIGGIKLRHQQWSLVMESVVPSDRGNYTCVVENKYGNIRHTYQLDVLERSPHRPILQAGLPANQTVVVGSNVEFHCKVYSDAQPHIQWLKHVEVNGSKYGPDGTPYVTVLKSWISKNAEADANLNLFNVTEQDEGEYLCRANNFVGIAEKPFWLHIRKPKPAEELMEMDDSGSVYAGILSYGTGLVLFILVLVIVIICRMKMPNKKAMNTTTVQKVSKFPLKRQVTVSLESNSSMNSNTPLVRITRLSSSDGPMLANVSELELPPDPKWELARSRLTLGKPLGEGCFGQVVMAEAIGIDKDKPNKAITVAVKMLKDDATDKDLSDLVSEMEMMKMIGKHKNIIKLLGACTQDGPLYVLVEYASKGNLREYLRARRPPGMDYSFDTCKLPEEQLTFKDLVSCAYQVARGMEYLASQKCIHRDLAARNVLVTEDNVMKIADFGLARDVHNIDYYKKTTNGRLPVKWMAPEALFDRVYTHQSDVWSFGVLLWEIFTLGGSPYPGIPVEELFKLLKEGHRMDKPANCTHDLYMIMRECWHAVPSQRPTFKQLVEDLDRVLTMTSTDEYLDLSVPFEQYSPAGQDTHSTCSSGDDSVFAHDLLPDEPCLPKHVPCNGVIRT

SEQ ID NO: 43 - protein, 812 aa, synthetic construct
MSEAGGGAAAAASLPRSRAGGMRAAWGSVWCLCLAAAVGALPAARRRGAERSGGQAAEYLRSETAFLEELVFGSGDTIELSCNTQSSSVSVFWFKDGIGIAPSNRTHIGQKLLKIINVSYDDSGLYSCKPRHSNEVLGNFTVRVTGVPFWTRPDKMEKKLLAVPAANTVRFRCPAGGNPTPTIYWLKNGKEFKGEHRIGGIKLRHQQWSLVMESVVPSDRGNYTCVVENKYGNIRHTYQLDVLERSPHRPILQAGLPANQTVVVGSNVEFHCKVYSDAQPHIQWLKHVEVNGSKYGPDGTPYVTVLKSWISKNAEADANLNLFNVTEQDEGEYLCRANNFVGIAEKPFWLHIRKPKPAEELMEMDDSGSVYAGILSYGTGLVLFILVLVIVIICRMKMPNKKAMNTTTVQKVSKFPLKRQVTVSLESNSSMNSNTPLVRITRLSSSDGPMLANVSELELPPDPKWELARSRLTLGKPLGEGCFGQVVMAEAIGIDKDKPNKAITVAVKMLKDDATDKDLSDLVSEMEMMKMIGKHKNIINLLGACTQDGPLYVLVEYASKGNLREYLRARRPPGMDYSFDTCKLPEEQLTFKDLVSCAYQVARGMEYLASQKCIHRDLAARNVLVTEDNVMKIADFGLARDVHNIDYYKETTNGRLPVKWMAPEALFDRVYTHQSDVWSFGVLLWEIFTLGGSPYPGIPVEELFKLLKEGHRMDKPANCTHDLYMIMRECWHAVPSQRPTFKQLVEDLDRVLTMTSTDEYLDLSVPFEQYSPAGQDTHSTCSSGDDSVFAHDLLPDEPCLPKHVPCNGVIRT

SEQ ID NO: 44 - protein, 812 aa, synthetic construct
MSEAGGGAAAAASLPRSRAGGMRAAWGSVWCLCLAAAVGALPAARRRGAERSGGQAAEYLRSETAFLEELVFGSGDTIELSCNTQSSSVSVFWFKDGIGIAPSNRTHIGQKLLKIINVSYDDSGLYSCKPRHSNEVLGNFTVRVTGVPFWTRPDKMEKKLLAVPAANTVRFRCPAGGNPTPTIYWLKNGKEFKGEHRIGGIKLRHQQWSLVMESVVPSDRGNYTCVVENKYGNIRHTYQLDVLERSPHRPILQAGLPANQTVVVGSNVEFHCKVYSDAQPHIQWLKHVEVNGSKYGPDGTPYVTVLKSWISKNAEADANLNLFNVTEQDEGEYLCRANNFVGIAEKPFWLHIRKPKPAEELMEMDDSGSVYAGILSYGTGLVLFILVLVIVIICRMKMPNKKAMNTTTVQKVSKFPLKRQVTVSLESNSSMNSNTPLVRITRLSSSDGPMLANVSELELPPDPKWELARSRLTLGKPLGEGCFGQVVMAEAIGIDKDKPNKAITVAVKMLKDDATDKDLSDLVSEMEMMKMIGKHKNIIKLLGACTQDGPLYVLVEYASKGNLREYLRARRPPGMDYSFDTCKLPEEQLTFKDLVSCAYQVARGMEYLASQKCIHRDLAARNVLVTEDNVMKIADFGLARDVHNIDYYKETTNGRLPVKWMAPEALFDRVYTHQSDVWSFGVLLWEIFTLGGSPYPGIPVEELFKLLKEGHRMDKPANCTHDLYMIMRECWHAVPSQRPTFKQLVEDLDRVLTMTSTDEYLDLSVPFEQYSPAGQDTHSTCSSGDDSVFAHDLLPDEPCLPKHVPCNGVIRT

SEQ ID NO: 45 - protein, 432 aa, synthetic construct
MGSSKSKPKDPSQRRMKMPNKKAMNTTTVQKVSKFPLKRQVTVSLESNSSMNSNTPLVRITRLSSSDGPMLANVSELELPPDPKWELARSRLTLGKPLGEGCFGQVVMAEAIGIDKDKPNKAITVAVKMLKDDATDKDLSDLVSEMEMMKMIGKHKNIINLLGACTQDGPLYVLVEYASKGNLREYLRARRPPGMDYSFDTCKLPEEQLTFKDLVSCAYQVARGMEYLASQKCIHRDLAARNVLVTEDNVMKIADFGLARDVHNIDYYKETTNGRLPVKWMAPEALFDRVYTHQSDVWSFGVLLWEIFTLGGSPYPGIPVEELFKLLKEGHRMDKPANCTHDLYMIMRECWHAVPSQRPTFKQLVEDLDRVLTMTSTDEYLDLSVPFEQYSPAGQDTHSTCSSGDDSVFAHDLLPDEPCLPKHVPCNGVIRT

SEQ ID NO: 46 - protein, 802 aa, synthetic construct
MLPLRLVLAGLLVAAGSAASHRGEMEPELFESPLLESEEEHLLLDPGNALKLYCDVNQSGASVVWYKESRPLLPGPRVRLQQSVLEIAEVAYEDSGLYVCRARGTGEVLRNFTISVVDSLASGDDDEDSDGDGPHGDRSEEPVYVHRAPYWTHPHRMDKKLYAVPAGNTVKFRCPASGSPSPSIRWFKNGREFRGEHRIGGIRLRHQHWSLVMESVVPSDRGNYTCLVENRFGSIRYSYLLDVLERSPHRPILQAGLPANTTALVGSDVEFFCKVYSDAQPHIQWLKHIEVNGSSYGPDGVPYVQVLKTADINSSEVEVLYLRNVTMEDAGEYTCLAGNSIGLSYQSAWLTVLPEELVHEAEAPEAKYTDIIIYTSGSLAVAMALIIVVLCRMQTQSSKQPLEPMAVHKLSKFPLIRQFSLDSSSSGKSSTSLMRVTRLSSSCAPMLAGVVELDLPLDSKWEFPREKLVLGKPLGEGCFGQVVRAEAYGIDRQWPDRAVTVAVKMLKDNATDKDLADLISEMEMMKLMDKHKNIINLLGVCTQDGPLYVIVEFAAKGNLREYLRARRPPMPDYTFDITELHEEQLCFKDLVSCVYQVARGMEYLESRRCIHRDLAARNVLVTAENVMKIADFGLARDVHDIDYYKKTSNGRLPVKWMAPEALFDRVYTHQSDVWSFGILMWEIFTLGGSPYPGIPVEELFKLLKEGHRMDCPSNCTHELYMLMRECWHAVPSQRPTFKQLVEGLDKILAAISEEYLDLSMPFEQYSPSCEDTTSTCSSDDSVFTHDPLPLAPCLFACPSGRT

SEQ ID NO: 47 - protein, 802 aa, synthetic construct
MLPLRLVLAGLLVAAGSAASHRGEMEPELFESPLLESEEEHLLLDPGNALKLYCDVNQSGASVVWYKESRPLLPGPRVRLQQSVLEIAEVAYEDSGLYVCRARGTGEVLRNFTISVVDSLASGDDDEDSDGDGPHGDRSEEPVYVHRAPYWTHPHRMDKKLYAVPAGNTVKFRCPASGSPSPSIRWFKNGREFRGEHRIGGIRLRHQHWSLVMESVVPSDRGNYTCLVENRFGSIRYSYLLDVLERSPHRPILQAGLPANTTALVGSDVEFFCKVYSDAQPHIQWLKHIEVNGSSYGPDGVPYVQVLKTADINSSEVEVLYLRNVTMEDAGEYTCLAGNSIGLSYQSAWLTVLPEELVHEAEAPEAKCTDIIIYTSGSLAVAMALIIVVLCRMQTQSSKQPLEPMAVHKLSKFPLIRQFSLDSSSSGKSSTSLMRVTRLSSSCAPMLAGVVELDLPLDSKWEFPREKLVLGKPLGEGCFGQVVRAEAYGIDRQWPDRAVTVAVKMLKDNATDKDLADLISEMEMMKLMDKHKNIINLLGVCTQDGPLYVIVEFAAKGNLREYLRARRPPMPDYTFDITELHEEQLCFKDLVSCVYQVARGMEYLESRRCIHRDLAARNVLVTAENVMKIADFGLARDVHDIDYYKKTSNGRLPVKWMAPEALFDRVYTHQSDVWSFGILMWEIFTLGGSPYPGIPVEELFKLLKEGHRMDCPSNCTHELYMLMRECWHAVPSQRPTFKQLVEGLDKILAAISEEYLDLSMPFEQYSPSCEDTTSTCSSDDSVFTHDPLPLAPCLFACPSGRT

SEQ ID NO: 48 - protein, 802 aa, synthetic construct
MLPLRLVLAGLLVAAGSAASHRGEMEPELFESPLLESEEEHLLLDPGNALKLYCDVNQSGASVVWYKESRPLLPGPRVRLQQSVLEIAEVAYEDSGLYVCRARGTGEVLRNFTISVVDSLASGDDDEDSDGDGPHGDRSEEPVYVHRAPYWTHPHRMDKKLYAVPAGNTVKFRCPASGSPSPSIRWFKNGREFRGEHRIGGIRLRHQHWSLVMESVVPSDRGNYTCLVENRFGSIRYSYLLDVLERSPHRPILQAGLPANTTALVGSDVEFFCKVYSDAQPHIQWLKHIEVNGSSYGPDGVPYVQVLKTADINSSEVEVLYLRNVTMEDAGEYTCLAGNSIGLSYQSAWLTVLPEELVHEAEAPEAKYTDIIIYTSGSLAVAMALIIVVLCRMQTQSSKQPLEPMAVHKLSKFPLIRQFSLDSSSSGKSSTSLMRVTRLSSSCAPMLAGVVELDLPLDSKWEFPREKLVLGKPLGEGCFGQVVRAEAYGIDRQWPDRAVTVAVKMLKDNATDKDLADLISEMEMMKLMDKHKNIINLLGVCTQDGPLYVIVEFAAKGNLREYLRARRPPMPDYTFDITELHEEQLCFKDLVSCVYQVARGMEYLESRRCIHRDLAARNVLVTAENVMKIADFGLARDVHDIDYYKETSNGRLPVKWMAPEALFDRVYTHQSDVWSFGILMWEIFTLGGSPYPGIPVEELFKLLKEGHRMDCPSNCTHELYMLMRECWHAVPSQRPTFKQLVEGLDKILAAISEEYLDLSMPFEQYSPSCEDTTSTCSSDDSVFTHDPLPLAPCLFACPSGRT

SEQ ID NO: 49 - protein, 802 aa, synthetic construct
MLPLRLVLAGLLVAAGSAASHRGEMEPELFESPLLESEEEHLLLDPGNALKLYCDVNQSGASVVWYKESRPLLPGPRVRLQQSVLEIAEVAYEDSGLYVCRARGTGEVLRNFTISVVDSLASGDDDEDSDGDGPHGDRSEEPVYVHRAPYWTHPHRMDKKLYAVPAGNTVKFRCPASGSPSPSIRWFKNGREFRGEHRIGGIRLRHQHWSLVMESVVPSDRGNYTCLVENRFGSIRYSYLLDVLERSPHRPILQAGLPANTTALVGSDVEFFCKVYSDAQPHIQWLKHIEVNGSSYGPDGVPYVQVLKTADINSSEVEVLYLRNVTMEDAGEYTCLAGNSIGLSYQSAWLTVLPEELVHEAEAPEAKCTDIIIYTSGSLAVAMALIIVVLCRMQTQSSKQPLEPMAVHKLSKFPLIRQFSLDSSSSGKSSTSLMRVTRLSSSCAPMLAGVVELDLPLDSKWEFPREKLVLGKPLGEGCFGQVVRAEAYGIDRQWPDRAVTVAVKMLKDNATDKDLADLISEMEMMKLMDKHKNIINLLGVCTQDGPLYVIVEFAAKGNLREYLRARRPPMPDYTFDITELHEEQLCFKDLVSCVYQVARGMEYLESRRCIHRDLAARNVLVTAENVMKIADFGLARDVHDIDYYKETSNGRLPVKWMAPEALFDRVYTHQSDVWSFGILMWEIFTLGGSPYPGIPVEELFKLLKEGHRMDCPSNCTHELYMLMRECWHAVPSQRPTFKQLVEGLDKILAAISEEYLDLSMPFEQYSPSCEDTTSTCSSDDSVFTHDPLPLAPCLFACPSGRT

SEQ ID NO: 50 - protein, 1363 aa, synthetic construct
MKSGAGGGTLAVFCGLLLAFAALCLCPTNGEICGPNVDIRNDIHELKRLENCTVVEGFLQILLISKAEDYRNFRFPKLTVITDYLLLFRVAGLESLSDLFPNLTVIRGRNLFYNYALVIFEMTNLKEIGLHNLRNITRGAIRIEKNSDLCYLSTVDWSLILDAVSNNYIVGNKPPKECGDLCPGTMEEKPLCEKTSINNEYNYRCWTTNHCQKMCPSSCGKRACTDQNECCHPECLGSCTAPDNNTACVACRNYYYEGVCMPTCPPNTYKFEGWRCVTKEFCSKVPATETSDYERFVIHNDECMAECPSGFIRNGSQSMFCSPCEGPCPKICEDGKTKTIDSVTSAQMLQGCTILKGNLLINIRRGNNIASELENFMGLIETVTGYVKIRHSHALVSLSFLKNLRYILGEEQVDGNYSFYVLDNHNLQQLWDWNHHNLTIKEGKMYFAFNPKLCVSEIYRMEEVSGTKGRQSKGDINPRNNGERASCESHILRFVSNTTLKNRIKLTWERYRPPDYRDLISFTVYYKEAPFKNVTEYDGQDACGSNSWNMVDVDLPPNKENDPGILLQGLKPWTQYAIYVKAVTLTMMENHHIHGAKSEIVYIRTNAAVPSIPLDVISASNSSSQLIVKWNPPSLPNGNLSYYIVRWQQQPQDSYLYRHNYCSKDKVPIRRYADGTIDTEEATEPTKPEGCGGEKGPCCACPKTEAEKQAEKEEAEYRKVFENFLHNSIFVPRPDRKRRDVFRIANATLATRNRNITGADHFTNASDAEESEVEYPFFETKVDGKERTVISHLQPFTLYRIDIHSCNHEADTLGCSASNFVFARTMPSEGADNIPGTVAWEAKEENTVYLKWLEPTNPNGLILMYEIKYGQHGEEKRECVSRQEYKKLGGAKLTHLNPGNYSARVQATSLAGNGSWTEPVSFYVQPKSANYDNFLHLIIVLPIAFLLIIGGLLIMLYVFNKKRNSDRLGNGVLYASVNPEYFSASDVYVPDEWEVPREKITMCRELGQGSFGMVYEGIAKGVVKDEPETRVAIKTVNESASMRERIEFLNEASVMKEFNCHHVVRLLGVVSQGQPTLVIMELMTRGDLKSYLRSLRPDTESNPGQAPPTLKKMIQMAGEIADGMAYLNANKFVHRDLAARNCMVAEDFTVKIGDFGMTRDIYETDYYRKGGKGLLPVRWMSPESLKDGVFTTHSDVWSFGVVLWEIATLAEQPYQGMTNEQVLRFVMEGGLLEKPDNCPDMLFELMRMCWQYNPKMRPSFLEIISSIKDELDPAFKEVSFFYSEENKPPDTEELDLETENMESIPLDPSSTLQPTDKHSGHKAENGPGVVVLRASFEERQPYAHMNGGRKNERALPLPQSSAC

SEQ ID NO: 51 - protein, 1363 aa, synthetic construct
MKSGAGGGTLAVFCGLLLAFAALCLCPTNGEICGPNVDIRNDIHELKRLENCTVVEGFLQILLISKAEDYRNFRFPKLTVITDYLLLFRVAGLESLSDLFPNLTVIRGRNLFYNYALVIFEMTNLKEIGLHNLRNITRGAIRIEKNSDLCYLSTVDWSLILDAVSNNYIVGNKPPKECGDLCPGTMEEKPLCEKTSINNEYNYRCWTTNHCQKMCPSSCGKRACTDQNECCHPECLGSCTAPDNNTACVACRNYYYEGVCMPTCPPNTYKFEGWRCVTKEFCSKVPATETSDYERFVIHNDECMAECPSGFIRNGSQSMFCSPCEGPCPKICEDGKTKTIDSVTSAQMLQGCTILKGNLLINIRRGNNIASELENFMGLIETVTGYVKIRHSHALVSLSFLKNLRYILGEEQVDGNYSFYVLDNHNLQQLWDWNHHNLTIKEGKMYFAFNPKLCVSEIYRMEEVSGTKGRQSKGDINPRNNGERASCESHILRFVSNTTLKNRIKLTWERYRPPDYRDLISFTVYYKEAPFKNVTEYDGQDACGSNSWNMVDVDLPPNKENDPGILLQGLKPWTQYAIYVKAVTLTMMENHHIHGAKSEIVYIRTNAAVPSIPLDVISASNSSSQLIVKWNPPSLPNGNLSYYIVRWQQQPQDSYLYRHNYCSKDKVPIRRYADGTIDTEEATEPTKPEGCGGEKGPCCACPKTEAEKQAEKEEAEYRKVFENFLHNSIFVPRPDRKRRDVFRIANATLATRNRNITGADHFTNASDAEESEVEYPFFETKVDGKERTVISHLQPFTLYRIDIHSCNHEADTLGCSASNFVFARTMPSEGADNIPGTVAWEAKEENTVYLKWLEPTNPNGLILMYEIKYGQHGEEKRECVSRQEYKKLGGAKLTHLNPGNYSARVQATSLAGNGSWTEPVSFYVQPKSANYDNFLHLIIVLPIAFLLIIGGLLIMLYVFNKKRNSDRLGNGVLYASVNPEYFSASDVYVPDEWEVPREKITMCRELGQGSFGMVYEGIAKGVVKDEPETRVAIKTVNESASMRERIEFLNEASVMKEFNCHHVVRLLGVVSQGQPTLVIMELMTRGDLKSYLRSLRPDTESNPGQAPPTLKKMIQMAGEIADGMAYLNANKFVHRDLAARNCMVAEDFTVKIGDFGMTRDIYETDYYRKGGKGLLPVRWMSPESLKDGVFTTHSDVWSFGVVLWEIATLAEQPYQGMTNEQVLRFVMEGGLLEKPDNCPDMLFELMRMCWQYNPKMRPSFLEIISSIKDELDPAFKEVSFFYSEENKPPDTEELDLETENMESIPLDPSSTLQPTDKHSGHKAENGPGVVVLRASFEERQPYAHMNGGRKNEHALPLPQSSAC

SEQ ID NO: 52 - protein, 1087 aa, synthetic construct
MGTPPRTFLILGCFLTGPLLTLCQLPLPTIVPNRNEMVVQLNSNFTLKCSGDSEVSWQYPVTEGSHRIDIRHEENNSGLFVTVLEVGNASAAHTGMYVCYYNHTQVEDGEVEGKDIYIYVPDPDMPFVPSLPEDQFILVEEGDPTVIPCRTSDPSAEVTLVNSLDKPVYAFYDSKQGFVGNFLAGPYTCKTMVKGVEFKSDEFLIYILRATSQLPVEIEALKTVYKTGETIVVTCVVFDNEVVNLQWNYPGKVKEKGLIKLDDIKVPSQKLVYTLTIPDASVKDTGDYECTARHATKEVKENKKVVITVHDKGFIHLEPQFSPLEAVNLHEVKNFVVDVQAYPAPKMYWLKDNVTLIENLTEIVTSSNRVQETRFQSVLKLIRAKEEDSGYYTLVAENEDEIKRYTFSLLIQVPALILDLMDDHQGSAGRQTVRCLAEGTPLPDVEWLVCKDIKKCSNDTSWTLLTNNISDIHMEAHLDERNMVESQVTFQKVEETLAVRCVARNDLGAVTRELKLVAPTLRSELTVAAAVLVLLVIVIISLIVLVIIWKQKPRYEIRWRVIESISPDGHEYIYVDPMQLPYDSRWEFPRDGLVLGRILGSGAFGKVVEGTAYGLSRSQPVMKVAVKMLKPTARSSEKQALMSELKIMTHLGPHLNIVNLLGACTKSGPIYIITEYCFYGDLVNYLHKNRDNFLSRHPEKPKKDLDIFGMNPADESTRSYVILSFENTGEYMDMKQADTTQYVPMLERKEGSKYSDIQRSVYDRPASYKKKSLSESEVKNLLSDDGSEGLSLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATNEVYEIMVKCWNSEPEKRPSFYHLSEIVESLLPGEYKKSYEKIHLDFLKSDHPAVTRMRGDCDNAYIGVTYKNEDKIKDRESGFDEQRLSADSGYIIPLPDIDPVSEDELGKRNRHSSQTSEESAIETGSSSSTFIKREDETIEDIDMMDDIGIDSSDLVEDSFL

SEQ ID NO: 53 - protein, 1087 aa, synthetic construct
MGTPPRTFLILGCFLTGPLLTLCQLPLPTIVPNRNEMVVQLNSNFTLKCSGDSEVSWQYPVTEGSHRIDIRHEENNSGLFVTVLEVGNASAAHTGMYVCYYNHTQVEDGEVEGKDIYIYVPDPDMPFVPSLPEDQFILVEEGDPTVIPCRTSDPSAEVTLVNSLDKPVYAFYDSKQGFVGNFLAGPYTCKTMVKGVEFKSDEFLIYILRATSQLPVEIEALKTVYKTGETIVVTCVVFDNEVVNLQWNYPGKVKEKGLIKLDDIKVPSQKLVYTLTIPDASVKDTGDYECTARHATKEVKENKKVVITVHDKGFIHLEPQFSPLEAVNLHEVKNFVVDVQAYPAPKMYWLKDNVTLIENLTEIVTSSNRVQETRFQSVLKLIRAKEEDSGYYTLVAENEDEIKRYTFSLLIQVPALILDLMDDHQGSAGRQTVRCLAEGTPLPDVEWLVCKDIKKCSNDTSWTLLTNNISDIHMEAHLDERNMVESQVTFQKVEETLAVRCVARNDLGAVTRELKLVAPTLRSELTVAAAVLVLLVIVIISLIVLVIIWKQKPRYEIRWRVIESISPDGHEYIYVDPMQLPYDSRWEFPRDGLVLGRILGSGAFGKVVEGTAYGLSRSQPVMKVAVKMLKPTARSSEKQALMSELKIMTHLGPHLNIVNLLGACTKSGPIYIITEYCFYGDLVNYLHKNRDNFLSRHPEKPKKDLDIFGMNPADESTRSYVILSFENTGEYMDMKQADTTQYVPMLERKEGSKYSDIQRSVYDRPASYKKKSLSESEVKNLLSDDGSEGLSLLDLLSFTYQVARGMEFLASKNCVHRDLAARNVLLAQGKIVKICDFGLARVIMHDSNYVSKGSTFLPVKWMAPESIFDNLYTTLSDVWSYGILLWEIFSLGGTPYPGMMVDSTFYNKIKSGYRMAKPDHATNEVYEIMVKCWNSEPEKRPSFYHLSEIVESLLPGEYKKSYEKIHLDFLKSDHPAVTRMRGDCDNAYIGVTYKNEDKIKDRESGFDEQRLSADSGYIIPLPDIDPVSEDELGKRNRHSSQTSEESAIETGSSSSTFIKREDETIEDIDMMDDIGIDSSDLVEDSFL

SEQ ID NO: 54 - protein, 1090 aa, synthetic construct
MLCPSLKASLQLLILTGLLEVTSGGSGLHIEPEDAELVLRLHSTFSLVCYGDGTLVWERDGQPLTAVLEHRDGVFISNLTLRNVTGRHTGEYACFYSPDQAPERAERKALYIYVPDPSLVFLPAITSEEFFIFITGYTEATIPCRVTNPELQVTLYEKKVENPIPATYDPQQGFKGFFEDKTYYCQAIVDDQEVDSDTFYVYRIQVSSVNVSISAVQTVVRQGENVTLMCTVSGNELVNFNWDYPRKQAGKAVEPVTDFLPGSTHDIRSILIIQNAELEDSGTYVCNVSEGYHEKTDRKDITVQVIERGFVRFHTHLASTVYAEVHKSHIIQVDVEAYPQPNIVWLKNNKTLTMESSSEFTITNRNLSETRYQTSLVLVRVKQEEGGYYTIRASNEDDAQELSFHLQINVPAKVVDLKENSSASSGEQTVTCSAEGMPQPEISWSTCSNIKWCGSQGQPTQLLGNNSAEIGLHTNATYHAELQVYRVNSTLQLHRVDEPLLLRCTVQNFLGSNSQDITLVPNALPFKVVIISVILALLVLTVISLIILIILWQKKPRYEIRWKVIESVSSDGHEYIYVDPMQLPYDSSWEVPRDKLVLGRTLGSGAFGRVVEATAHGLSHSQSTMKVAVKMLKSTARSSEKQALMSELKIMSHLGPHLNIVNLLGACTKGGPIYIITEYCRYGDLVDYLHRNKHTFLQSYGEKARREAELYGNTIKEDHVQSHLSLSVESDGGYMDMSKDESLDYVPMSDMKGEVKYADIESSNYGTPYELDSYSPSAPERTDRVTLINESPLLSYMDLVGFSFQVANGMEFLASKNCVHRDLAARNVLICEGKLVKICDFGLARDIMRDSNYISKGSTFLPLKWMAPESIFNNLYTTLSDVWSFGILLWEIFTLGGTPYPELPMNEQFYNAIKRGYRMSKPTHASDEIYDIMQKCWEEKFEIRPSFSQLVVLMGNLLVDCYRKRYQQVDEEFMKSDHPAVVRTRPTIPGLNNARLPPSSPTLYTAVHQNGGENDYIIPLPDPKPDAICDLPQEASVSRASSMLNEANTSSTISCDSPLGPRQDEEPECDLQLGCQELAPGHHEVEESFL

SEQ ID NO: 55 - protein, 1090 aa, synthetic construct
MLCPSLKASLQLLILTGLLEVTSGGSGLHIEPEDAELVLRLHSTFSLVCYGDGTLVWERDGQPLTAVLEHRDGVFISNLTLRNVTGRHTGEYACFYSPDQAPERAERKALYIYVPDPSLVFLPAITSEEFFIFITGYTEATIPCRVTNPELQVTLYEKKVENPIPATYDPQQGFKGFFEDKTYYCQAIVDDQEVDSDTFYVYRIQVSSVNVSISAVQTVVRQGENVTLMCTVSGNELVNFNWDYPRKQAGKAVEPVTDFLPGSTHDIRSILIIQNAELEDSGTYVCNVSEGYHEKTDRKDITVQVIERGFVRFHTHLASTVYAEVHKSHIIQVDVEAYPQPNIVWLKNNKTLTMESSSEFTITNRNLSETRYQTSLVLVRVKQEEGGYYTIRASNEDDAQELSFHLQINVPAKVVDLKENSSASSGEQTVTCSAEGMPQPEISWSTCSNIKWCGSQGQPTQLLGNNSAEIGLHTNATYHAELQVYRVNSTLQLHRVDEPLLLRCTVQNFLGSNSQDITLVPNALPFKVVIISVILALLVLTVISLIILIILWQKKPRYEIRWKAIESVSSDGHEYIYVDPMQLPYDSSWEVPRDKLVLGRTLGSGAFGRVVEATAHGLSHSQSTMKVAVKMLKSTARSSEKQALMSELKIMSHLGPHLNIVNLLGACTKGGPIYIITEYCRYGDLVDYLHRNKHTFLQSYGEKARREAELYGNTIKEDHVQSHLSLSVESDGGYMDMSKDESLDYVPMSDMKGEVKYADIESSNYGTPYELDSYSPSAPERTDRVTLINESPLLSYMDLVGFSFQVANGMEFLASKNCVHRDLAARNVLICEGKLVKICDFGLARDIMRDSNYISKGSTFLPLKWMAPESIFNNLYTTLSDVWSFGILLWEIFTLGGTPYPELPMNEQFYNAIKRGYRMSKPTHASDEIYDIMQKCWEEKFEIRPSFSQLVVLMGNLLVDCYRKRYQQVDEEFMKSDHPAVVRTRPTIPGLNNARLPPSSPTLYTAVHQNGGENDYIIPLPDPKPDAICDLPQEASVSRASSMLNEANTSSTISCDSPLGPRQDEEPECDLQLGCQELAPGHHEVEESFL

SEQ ID NO: 56 - protein, 1090 aa, synthetic construct
MLCPSLKASLQLLILTGLLEVTSGGSGLHIEPEDAELVLRLHSTFSLVCYGDGTLVWERDGQPLTAVLEHRDGVFISNLTLRNVTGRHTGEYACFYSPDQAPERAERKALYIYVPDPSLVFLPAITSEEFFIFITGYTEATIPCRVTNPELQVTLYEKKVENPIPATYDPQQGFKGFFEDKTYYCQAIVDDQEVDSDTFYVYRIQVSSVNVSISAVQTVVRQGENVTLMCTVSGNELVNFNWDYPRKQAGKAVEPVTDFLPGSTHDIRSILIIQNAELEDSGTYVCNVSEGYHEKTDRKDITVQVIERGFVRFHTHLASTVYAEVHKSHIIQVDVEAYPQPNIVWLKNNKTLTMESSSEFTITNRNLSETRYQTSLVLVRVKQEEGGYYTIRASNEDDAQELSFHLQINVPAKVVDLKENSSASSGEQTVTCSAEGMPQPEISWSTCSNIKWCGSQGQPTQLLGNNSAEIGLHTNATYHAELQVYRVNSTLQLHRVDEPLLLRCTVQNFLGSNSQDITLVPNALPFKVVIISVILALLVLTVISLIILIILWQKKPRYEIRWKVIESVSSDGHEYIYVDPMQLPYDSSWEVPRDKLVLGRTLGSGAFGRVVEATAHGLSHSQSTMKVAVKMLKSTARSSEKQALMSELKIMSHLGPHLNIVNLLGACTKGGPIYIITEYCRYGDLVDYLHRNKHTFLQSYGEKARREAELYGNTIKEDHVQSHLSLSVESDGGYMDMSKDESLDYVPMSDMKGEVKYADIESSNYGTPYELDSYSPSAPERTDRVTLINESPLLSYMDLVGFSFQVANGMEFLASKNCVHRDLAARNVLICEGKLVKICDFGLARDIMRNSNYISKGSTFLPLKWMAPESIFNNLYTTLSDVWSFGILLWEIFTLGGTPYPELPMNEQFYNAIKRGYRMSKPTHASDEIYDIMQKCWEEKFEIRPSFSQLVVLMGNLLVDCYRKRYQQVDEEFMKSDHPAVVRTRPTIPGLNNARLPPSSPTLYTAVHQNGGENDYIIPLPDPKPDAICDLPQEASVSRASSMLNEANTSSTISCDSPLGPRQDEEPECDLQLGCQELAPGHHEVEESFL

SEQ ID NO: 57 - protein, 1108 aa, synthetic construct
MADYKDDDDKKGGIIVAILLLIVMLAIEILLLITLIIAVTSGGSGLHIEPEDAELVLRLHSTFSLVCYGDGTLVWERDGQPLTAVLEHRDGVFISNLTLRNVTGRHTGEYACFYSPDQAPERAERKALYIYVPDPSLVFLPAITSEEFFIFITGYTEATIPCRVTNPELQVTLYEKKVENPIPATYDPQQGFKGFFEDKTYYCQAIVDDQEVDSDTFYVYRIQVSSVNVSISAVQTVVRQGENVTLMCTVSGNELVNFNWDYPRKQAGKAVEPVTDFLPGSTHDIRSILIIQNAELEDSGTYVCNVSEGYHEKTDRKDITVQVIERGFVRFHTHLASTVYAEVHKSHIIQVDVEAYPQPNIVWLKNNKTLTMESSSEFTITNRNLSETRYQTSLVLVRVKQEEGGYYTIRASNEDDAQELSFHLQINVPAKVVDLKENSSASSGEQTVTCSAEGMPQPEISWSTCSNIKWCGSQGQPTQLLGNNSAEIGLHTNATYHAELQVYRVNSTLQLHRVDEPLLLRCTVQNFLGSNSQDITLVPNALPFKVVIISVILALLVLTVISLIILIILWQKKPRYEIRWKVIESVSSDGHEYIYVDPMQLPYDSSWEVPRDKLVLGRTLGSGAFGRVVEATAHGLSHSQSTMKVAVKMLKSTARSSEKQALMSELKIMSHLGPHLNIVNLLGACTKGGPIYIITEYCRYGDLVDYLHRNKHTFLQSYGEKARREAELYGNTIKEDHVQSHLSLSVESDGGYMDMSKDESLDYVPMSDMKGEVKYADIESSNYGTPYELDSYSPSAPERTDRVTLINESPLLSYMDLVGFSFQVANGMEFLASKNCVHRDLAARNVLICEGKLVKICDFGLARDIMRDSNYISKGSTFLPLKWMAPESIFNNLYTTLSDVWSFGILLWEIFTLGGTPYPELPMNEQFYNAIKRGYRMSKPTHASDEIYDIMQKCWEEKFEIRPSFSQLVVLMGNLLVDCYRKRYQQVDEEFMKSDHPAVVRTRPTIPGLNNARLPPSSPTLYTAVHQNGGENDYIIPLPDPKPDAICDLPQEASVSRASSMLNEANTSSTISCDSPLGPRQDEEPECDLQLGCQELAPGHHEVEESFL

SEQ ID NO: 58 - protein, 1100 aa, synthetic construct
MAKGGIIVAILLLIVMLAIEILLLITLIIAVTSGGSGLHIEPEDAELVLRLHSTFSLVCYGDGTLVWERDGQPLTAVLEHRDGVFISNLTLRNVTGRHTGEYACFYSPDQAPERAERKALYIYVPDPSLVFLPAITSEEFFIFITGYTEATIPCRVTNPELQVTLYEKKVENPIPATYDPQQGFKGFFEDKTYYCQAIVDDQEVDSDTFYVYRIQVSSVNVSISAVQTVVRQGENVTLMCTVSGNELVNFNWDYPRKQAGKAVEPVTDFLPGSTHDIRSILIIQNAELEDSGTYVCNVSEGYHEKTDRKDITVQVIERGFVRFHTHLASTVYAEVHKSHIIQVDVEAYPQPNIVWLKNNKTLTMESSSEFTITNRNLSETRYQTSLVLVRVKQEEGGYYTIRASNEDDAQELSFHLQINVPAKVVDLKENSSASSGEQTVTCSAEGMPQPEISWSTCSNIKWCGSQGQPTQLLGNNSAEIGLHTNATYHAELQVYRVNSTLQLHRVDEPLLLRCTVQNFLGSNSQDITLVPNALPFKVVIISVILALLVLTVISLIILIILWQKKPRYEIRWKVIESVSSDGHEYIYVDPMQLPYDSSWEVPRDKLVLGRTLGSGAFGRVVEATAHGLSHSQSTMKVAVKMLKSTARSSEKQALMSELKIMSHLGPHLNIVNLLGACTKGGPIYIITEYCRYGDLVDYLHRNKHTFLQSYGEKARREAELYGNTIKEDHVQSHLSLSVESDGGYMDMSKDESLDYVPMSDMKGEVKYADIESSNYGTPYELDSYSPSAPERTDRVTLINESPLLSYMDLVGFSFQVANGMEFLASKNCVHRDLAARNVLICEGKLVKICDFGLARDIMRDSNYISKGSTFLPLKWMAPESIFNNLYTTLSDVWSFGILLWEIFTLGGTPYPELPMNEQFYNAIKRGYRMSKPTHASDEIYDIMQKCWEEKFEIRPSFSQLVVLMGNLLVDCYRKRYQQVDEEFMKSDHPAVVRTRPTIPGLNNARLPPSSPTLYTAVHQNGGENDYIIPLPDPKPDAICDLPQEASVSRASSMLNEANTSSTISCDSPLGPRQDEEPECDLQLGCQELAPGHHEVEESFL

SEQ ID NO: 59 - protein, 237 aa, synthetic construct
MWIKNVGLLCVLILVSQMLLASCERQKERRRGKQGIEHGGKKQAESNPEREKGRKPKGGKASPKGKFKSKENADCSWAVTDMSAATVHIECRNGDSAFWCEFSGDPSACPHYAANQKSYWKQVSRSLKKQKQICQDPRSILKPKICRKGPRGAHLKLTRSSLLAAVDPAKGHPAHHAAEDAQGPAASETGKQPEHSPPDCVEDVDYIDQRKVAEEYCPESLLSLCNFFITMVQDKKC

SEQ ID NO: 60 - protein, 189 aa, synthetic construct
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLPARTVETRQAQDLARSYGIPYIETSAKTRQGVEDAFYTLVREIRQHKLRKLNPPDESGPGCMNCKCVIS

SEQ ID NO: 61 - DNA, 477 bp, synthetic construct
  source 1..477: mol_type = other DNA
atggcggcaggagcagctggttccataaccacattgcccgccctcccagacgatgggggcggaggcgcatttcctccaggtcatttcaaggaccccaaaaggctgtactgtaagaatggtggttttttcctgaggataaaccctgacggacgggtggacggcgtacgcgagaaatcagatccacatatcaagctgcagttgcaagccgaggaacgcggggtagtctctataaaaggggttagcgctaacagatttttggcaatgaaggaagacggtaggctcctcgcgcttaagtgtgccaccgaggagtgcttcttcttcgaacggctcgaatctaacaactacaacacgtaccgctctcgcaaatactctgactggtacgtcgcactcaaacgcactgggcagtataaaccggggccaaagacgggtccggggcagaaagctatcctgttccttccaatgtccgccaagagttag

SEQ ID NO: 62 - DNA, 477 bp, synthetic construct; identical to SEQ ID NO: 61
  source 1..477: mol_type = other DNA

SEQ ID NO: 63 - DNA, 540 bp, synthetic construct
  source 1..540: mol_type = other DNA
atgatgtgtaaagttcttatcttcggatgcatctccgtagcaatgctcatgactacagcttacatggcggcaggagcagctggttccataaccacattgcccgccctcccagacgatgggggcggaggcgcatttcctccaggtcatttcaaggaccccaaaaggctgtactgtaagaatggtggttttttcctgaggataaaccctgacggacgggtggacggcgtacgcgagaaatcagatccacatatcaagctgcagttgcaagccgaggaacgcggggtagtctctataaaaggggttagcgctaacagatttttggcaatgaaggaagacggtaggctcctcgcgcttaagtgtgccaccgaggagtgcttcttcttcgaacggctcgaatctaacaactacaacacgtaccgctctcgcaaatactctgactggtacgtcgcactcaaacgcactgggcagtataaaccggggccaaagacgggtccggggcagaaagctatcctgttccttccaatgtccgccaagagttag

SEQ ID NO: 64 - DNA, 528 bp, synthetic construct
  source 1..528: mol_type = other DNA
atgggggtcaaagtgctgtttgccttgatctgtattgctgtggccgaggcaatggcggcaggagcagctggttccataaccacattgcccgccctcccagacgatgggggcggaggcgcatttcctccaggtcatttcaaggaccccaaaaggctgtactgtaagaatggtggttttttcctgaggataaaccctgacggacgggtggacggcgtacgcgagaaatcagatccacatatcaagctgcagttgcaagccgaggaacgcggggtagtctctataaaaggggttagcgctaacagatttttggcaatgaaggaagacggtaggctcctcgcgcttaagtgtgccaccgaggagtgcttcttcttcgaacggctcgaatctaacaactacaacacgtaccgctctcgcaaatactctgactggtacgtcgcactcaaacgcactgggcagtataaaccggggccaaagacgggtccggggcagaaagctatcctgttccttccaatgtccgccaagagttag

SEQ ID NO: 65 - DNA, 537 bp, synthetic construct
  source 1..537: mol_type = other DNA
atgtatcgcatgcaactgctttcatgcattgctcttagcctggcgctggtcacgaactctatggcggcaggagcagctggttccataaccacattgcccgccctcccagacgatgggggcggaggcgcatttcctccaggtcatttcaaggaccccaaaaggctgtactgtaagaatggtggttttttcctgaggataaaccctgacggacgggtggacggcgtacgcgagaaatcagatccacatatcaagctgcagttgcaagccgaggaacgcggggtagtctctataaaaggggttagcgctaacagatttttggcaatgaaggaagacggtaggctcctcgcgcttaagtgtgccaccgaggagtgcttcttcttcgaacggctcgaatctaacaactacaacacgtaccgctctcgcaaatactctgactggtacgtcgcactcaaacgcactgggcagtataaaccggggccaaagacgggtccggggcagaaagctatcctgttccttccaatgtccgccaagagttag

SEQ ID NO: 66 - DNA, 534 bp, synthetic construct
  source 1..534: mol_type = other DNA
atgagaatgcaactgctcctgcttatagcgctcagtttggctctcgtgaccaactcaatggcggcaggagcagctggttccataaccacattgcccgccctcccagacgatgggggcggaggcgcatttcctccaggtcatttcaaggaccccaaaaggctgtactgtaagaatggtggttttttcctgaggataaaccctgacggacgggtggacggcgtacgcgagaaatcagatccacatatcaagctgcagttgcaagccgaggaacgcggggtagtctctataaaaggggttagcgctaacagatttttggcaatgaaggaagacggtaggctcctcgcgcttaagtgtgccaccgaggagtgcttcttcttcgaacggctcgaatctaacaactacaacacgtaccgctctcgcaaatactctgactggtacgtcgcactcaaacgcactgggcagtataaaccggggccaaagacgggtccggggcagaaagctatcctgttccttccaatgtccgccaagagttag

SEQ ID NO: 67 - DNA, 537 bp, synthetic construct
  source 1..537: mol_type = other DNA
atgaggcggatgcaattgctgctgttgatcgcactctctctggcacttgtcactaatagtatggcggcaggagcagctggttccataaccacattgcccgccctcccagacgatgggggcggaggcgcatttcctccaggtcatttcaaggaccccaaaaggctgtactgtaagaatggtggttttttcctgaggataaaccctgacggacgggtggacggcgtacgcgagaaatcagatccacatatcaagctgcagttgcaagccgaggaacgcggggtagtctctataaaaggggttagcgctaacagatttttggcaatgaaggaagacggtaggctcctcgcgcttaagtgtgccaccgaggagtgcttcttcttcgaacggctcgaatctaacaactacaacacgtaccgctctcgcaaatactctgactggtacgtcgcactcaaacgcactgggcagtataaaccggggccaaagacgggtccggggcagaaagctatcctgttccttccaatgtccgccaagagttag

SEQ ID NO: 68 - DNA, 627 bp, synthetic construct
  source 1..627: mol_type = other DNA
atgaattttacagaagggtgtgaagcgactggcaggagaccaggatccgccgggtcaaggagaagaagggccccccggcctgggcctgtcgcgcttcttcccctgttgcttccgctgttgcttccaccggcagctgcggttcccttgcccatggcggcaggagcagctggttccataaccacattgcccgccctcccagacgatgggggcggaggcgcatttcctccaggtcatttcaaggaccccaaaaggctgtactgtaagaatggtggttttttcctgaggataaaccctgacggacgggtggacggcgtacgcgagaaatcagatccacatatcaagctgcagttgcaagccgaggaacgcggggtagtctctataaaaggggttagcgctaacagatttttggcaatgaaggaagacggtaggctcctcgcgcttaagtgtgccaccgaggagtgcttcttcttcgaacggctcgaatctaacaactacaacacgtaccgctctcgcaaatactctgactggtacgtcgcactcaaacgcactgggcagtataaaccggggccaaagacgggtccggggcagaaagctatcctgttccttccaatgtccgccaagagttag

SEQ ID NO: 69 - DNA, 558 bp, synthetic construct
  source 1..558: mol_type = other DNA
atggcgctcacgtttgccctcttggttgcgcttctcgttctttcctgcaagagtagctgttccgtgggaatggtatccaagatggcggcaggagcagctggttccataaccacattgcccgccctcccagacgatgggggcggaggcgcatttcctccaggtcatttcaaggaccccaaaaggctgtactgtaagaatggtggttttttcctgaggataaaccctgacggacgggtggacggcgtacgcgagaaatcagatccacatatcaagctgcagttgcaagccgaggaacgcggggtagtctctataaaaggggttagcgctaacagatttttggcaatgaaggaagacggtaggctcctcgcgcttaagtgtgccaccgaggagtgcttcttcttcgaacggctcgaatctaacaactacaacacgtaccgctctcgcaaatactctgactggtacgtcgcactcaaacgcactgggcagtataaaccggggccaaagacgggtccggggcagaaagctatcctgttccttccaatgtccgccaagagttag

SEQ ID NO: 70 - DNA, 540 bp, synthetic construct
  source 1..540: mol_type = other DNA
atgtggtggcgcttgtggtggttgctgttgttgttgctgctgttgtggcctatggtctgggcaatggcggcaggagcagctggttccataaccacattgcccgccctcccagacgatgggggcggaggcgcatttcctccaggtcatttcaaggaccccaaaaggctgtactgtaagaatggtggttttttcctgaggataaaccctgacggacgggtggacggcgtacgcgagaaatcagatccacatatcaagctgcagttgcaagccgaggaacgcggggtagtctctataaaaggggttagcgctaacagatttttggcaatgaaggaagacggtaggctcctcgcgcttaagtgtgccaccgaggagtgcttcttcttcgaacggctcgaatctaacaactacaacacgtaccgctctcgcaaatactctgactggtacgtcgcactcaaacgcactgggcagtataaaccggggccaaagacgggtccggggcagaaagctatcctgttccttccaatgtccgccaagagttag

SEQ ID NO: 71 - DNA, 564 bp, synthetic construct
  source 1..564: mol_type = other DNA
atgaatagcttcagtacgtctgcgttcggacctgtggcttttagcctcggactgctgctcgtgctgccggcggcgtttccggcacccatggcggcaggagcagctggttccataaccacattgcccgccctcccagacgatgggggcggaggcgcatttcctccaggtcatttcaaggaccccaaaaggctgtactgtaagaatggtggttttttcctgaggataaaccctgacggacgggtggacggcgtacgcgagaaatcagatccacatatcaagctgcagttgcaagccgaggaacgcggggtagtctctataaaaggggttagcgctaacagatttttggcaatgaaggaagacggtaggctcctcgcgcttaagtgtgccaccgaggagtgcttcttcttcgaacggctcgaatctaacaactacaacacgtaccgctctcgcaaatactctgactggtacgtcgcactcaaacgcactgggcagtataaaccggggccaaagacgggtccggggcagaaagctatcctgttccttccaatgtccgccaagagttag

SEQ ID NO: 72 - DNA, 477 bp, synthetic construct
  source 1..477: mol_type = other DNA
atggcggcaggagcagctggttccataaccacattgcccgccctcccagacgatgggggcggaggcgcatttcctccaggtcatttcaaggaccccaaaaggctgtactgtaagaatggtggttttttcctgaggataaaccctgacggacgggtggacggcgtacgcgagaaatcagatccacatatcaagctgcagttgatagccgaggaacgcggggtagtctctataaaaggggttagcgctaacagatttttggcaatgaaggaagacggtaggctcctcgcgcttaagtgtgccaccgaggagtgcttcttcttcgaacggctcgaatctaacggctacaacacgtaccgctctcgcaaatactctgactggtacgtcgcactcaaacgcactgggcagtataaaccggggccaaagacgggtccggggcagaaagctatcctgttccttccaatgtccgccaagagttag

SEQ ID NO: 73 - DNA, 477 bp, synthetic construct
  source 1..477: mol_type = other DNA
atggcggcaggagcagctggttccataaccacattgcccgccctcccagacgatgggggcggaggcgcatttcctccaggtcatttcaaggaccccaaaaggctgtactgtaagaatggtggttttttcctgaggataaaccctgacggacgggtggacggcgtacgcgagaaatcagatccacatatcaagctgcagttgatagccgaggaacgcggggtagtctctataaaaggggttagcgctaacagatttttggcaatgaaggaagacggtaggctcctcgcgcttaagtctgccaccgaggagtgcttcttcttcgaacggctcgaatctaacggctacaacacgtaccgctctcgcaaatactctgactggtacgtcgcactcaaacgcactgggcagtataaaccggggccaaagacgggtccggggcagaaagctatcctgttccttccaatgtccgccaagagttag

SEQ ID NO: 74 - DNA, 531 bp, synthetic construct
  source 1..531: mol_type = other DNA
atgagaatgcaactgctcctgcttatagcgctcagtttggctctcgtgaccaactcagcggcaggagcagctggttccataaccacattgcccgccctcccagacgatgggggcggaggcgcatttcctccaggtcatttcaaggaccccaaaaggctgtactgtaagaatggtggttttttcctgaggataaaccctgacggacgggtggacggcgtacgcgagaaatcagatccacatatcaagctgcagttgcaagccgaggaacgcggggtagtctctataaaaggggttagcgctaacagatttttggcaatgaaggaagacggtaggctcctcgcgcttaagtgtgccaccgaggagtgcttcttcttcgaacggctcgaatctaacaactacaacacgtaccgctctcgcaaatactctgactggtacgtcgcactcaaacgcactgggcagtataaaccggggccaaagacgggtccggggcagaaagctatcctgttccttccaatgtccgccaagagttag

SEQ ID NO: 75 - DNA, 477 bp, synthetic construct
  source 1..477: mol_type = other DNA
atggcggcaggagcagctggttccataaccacattgcccgccctcccagacgatgggggcggaggcgcatttcctccaggtcatttcaaggaccccaaaaggctgtactgtaagaatggtggttttttcctgaggataaaccctgacggacgggtggacggcgtacgcgagaaatcagatccacatatcaagctgcagttgcaagccgaggaacgcggggtagtctctataaaaggggtttgcgctaacagatatttggcaatgaaggaagacggtaggctcctcgcgcttaagtgtgccaccgaggagtgcttcttcttcgaacggctcgaatctaacaactacaacacgtaccgctctcgcaaatactctgactggtacgtcgcactcaaacgcactgggcagtataaaccggggccaaagacgggtccggggcagaaagctatcctgttccttccaatgtccgccaagagttag

SEQ ID NO: 76 - DNA, 477 bp, synthetic construct
  source 1..477: mol_type = other DNA
atggcggcaggagcagctggttccataaccacattgcccgccctcccagacgatgggggcggaggcgcatttcctccaggtcatttcaaggaccccaaaaggctgtactgtaagaatggtggttttttcctgctgataaaccctgacggacgggtggacggcacccgcgagaaatcagatccattcatcaagctgcagttgcaagccgaggaacgcggggtagtctctataaaaggggttagcgctaacagatttttggcaatgaaggaagacggtaggctctacgcgcttaagtatgccaccgaggagtgcttcttcttcgaacggctcgaagagaacaactacaacacgtaccgctctcgcaaatactctgactggtacgtcgcactcaaacgcactgggcagtataaaccggggccaaagacgggtccggggcagaaagctatcctgttccttccaatgtccgccaagagttag

SEQ ID NO: 77 - DNA, 540 bp, synthetic construct
  source 1..540: mol_type = other DNA
atgatgtgtaaagttcttatcttcggatgcatctccgtagcaatgctcatgactacagcttacatggcggcaggagcagctggttccataaccacattgcccgccctcccagacgatgggggcggaggcgcatttcctccaggtcatttcaaggaccccaaaaggctgtactgtaagaatggtggttttttcctgctgataaaccctgacggacgggtggacggcacccgcgagaaatcagatccattcatcaagctgcagttgcaagccgaggaacgcggggtagtctctataaaaggggttagcgctaacagatttttggcaatgaaggaagacggtaggctctacgcgcttaagtatgccaccgaggagtgcttcttcttcgaacggctcgaagagaacaactacaacacgtaccgctctcgcaaatactctgactggtacgtcgcactcaaacgcactgggcagtataaaccggggccaaagacgggtccggggcagaaagctatcctgttccttccaatgtccgccaagagttag

SEQ ID NO: 78 - DNA, 537 bp, synthetic construct
  source 1..537: mol_type = other DNA
atgtatcgcatgcaactgctttcatgcattgctcttagcctggcgctggtcacgaactctatggcggcaggagcagctggttccataaccacattgcccgccctcccagacgatgggggcggaggcgcatttcctccaggtcatttcaaggaccccaaaaggctgtactgtaagaatggtggttttttcctgctgataaaccctgacggacgggtggacggcacccgcgagaaatcagatccattcatcaagctgcagttgcaagccgaggaacgcggggtagtctctataaaaggggttagcgctaacagatttttggcaatgaaggaagacggtaggctctacgcgcttaagtatgccaccgaggagtgcttcttcttcgaacggctcgaagagaacaactacaacacgtaccgctctcgcaaatactctgactggtacgtcgcactcaaacgcactgggcagtataaaccggggccaaagacgggtccggggcagaaagctatcctgttccttccaatgtccgccaagagttag

SEQ ID NO: 79 - DNA, 477 bp, synthetic construct; identical to SEQ ID NO: 75
  source 1..477: mol_type = other DNA

SEQ ID NO: 80 - DNA, 477 bp, synthetic construct
  source 1..477: mol_type = other DNA
atggcggcaggagcagctggttccataaccacattgcccgccctcccagacgatgggggcggaggcgcatttcctccaggtcatttcaaggaccccaaaaggctgtactgtaagaatggtggttttttcctgctgataaaccctgacggacgggtggacggcacccgcgagaaatcagatccattcatcaagctgcagttgcaagccgaggaacgcggggtagtctctataaaaggggttagcgctaacagatttttggcaatgaaggaagacggtaggctctacgcgcttaagtgtgccaccgaggagtgcttcttcttcgaacggctcgaagagaacaactacaacacgtaccgctctcgcaaatactctgactggtacgtcgcactcaaacgcactgggcagtataaaccggggccaaagacgggtccggggcagaaagctatcctgttccttccaatgtccgccaagagttag

SEQ ID NO: 81 - DNA, 477 bp, synthetic construct
  source 1..477: mol_type = other DNA
atggcggcaggagcagctggttccataaccacattgcccgccctcccagacgatgggggcggaggcgcatttcctccaggtcatttcaaggaccccaaaaggctgtactgtaagaatggtggttttttcctgctgataaaccctgacggacgggtggacggcacccgcgacaaatcagatccattcatcaagctgcagttgcaagccgaggaacgcggggtagtctctataaaaggggttagcgctaacagatttttggcaatgaaggaagacggtaggctctacgcgataaagaatgccaccgaggagtgcttcttcttcgaacggctcgaagagaacaactacaacacgtaccgctctcgcaaataccctgactggtacgtcgcactcaaacgcactgggcagtataaaccggggccaaagacgggtccggggcagaaagctatcctgttccttccaatgtccgccaagagttag

SEQ ID NO: 82 - DNA, 477 bp, synthetic construct
  source 1..477: mol_type = other DNA
atggcggcaggagcagctggttccataaccacattgcccgccctcccagacgatgggggcggaggcgcatttcctccaggtcatttcaaggaccccaaaaggctgtactgtaagaatggtggttttttcctgctgataaaccctgacggacgggtggacggcacccgcgacaaatcagatccattcatcaagctgcagttgcaagccgaggaacgcggggtagtctctataaaaggggttagcgctaacagatttttggcaatgaaggaagacggtaggctctacgcgataaagtgtgccaccgaggagtgcttcttcttcgaacggctcgaagagaacaactacaacacgtaccgctctcgcaaataccctgactggtacgtcgcactcaaacgcactgggcagtataaaccggggccaaagacgggtccggggcagaaagctatcctgttccttccaatgtccgccaagagttag

SEQ ID NO: 83 - DNA, 468 bp, synthetic construct
  source 1..468: mol_type = other DNA
atggccgccgggagcatcaccacgctgccagccctgccggaggacggcggcagcggcgctttcccgccgggccacttcaaggaccccaagcggctgtactgcaagaacgggggcttcttcctgcgcatccaccccgacggccgagtggacggggtccgcgagaagagcgacccacacatcaaactacaacttcaagcagaagagagaggggttgtgtctatcaaaggagtgtgtgcaaaccgttaccttgctatgaaagaagatggaagattactagcttctaaatgtgttacagacgagtgtttcttttttgaacgattggagtctaataactacaatacttaccggtcaaggaaatactccagttggtatgtggcactgaaacgaactgggcagtataaacttggacccaaaacaggacctgggcagaaagctatactttttcttccaatgtctgctaagagctga

SEQ ID NO: 84 - DNA, 468 bp, synthetic construct
  source 1..468: mol_type = other DNA
atggccacaggagaaatcaccactctacccgccacacctgaagatggaggcagtggcggcttccctccaggaaactttaaggatcccaagaggctgtactgtaaaaacgggggctacttcttgagaataaactctaatggaagcgtggacgggatccgagagaagaacgacccccacatcaagcttcaactccaggcgacctcagtaggggaggtagtgatcaaaggggtctcagccaaccgttatctggccatgaatggagatggaagactgtttggaacgagacggacaacagatgaatgctacttcatggagaggctggagagtaacaactacaacacctaccgctcacggaagtaccctgacatgtatgtggcgctgaaaaggactggccagtacaagtcaggatccaaaactggaccgggccaaaaagccattctctttctccccatgtcagccagacgctga

SEQ ID NO: 85 - DNA, 570 bp, synthetic construct
  source 1..570: mol_type = other DNA
atgaccgagtacaagctggtggtagtgggagctgtaggtgtcgggaagagcgctttgacgatacagctcattcagaaccattttgttgatgagtacgaccccacaatagaggattcctacagaaagcaagtcgtcatcgatggagagacctgtttgctggacatcctggatacggcggggcaggaggagtacagtgccatgcgagaccagtacatgagaacgggggaaggattcctgtgcgtctttgccattaacaacaccaagtcctttgaggacatccaccagtacagggagcagatcaagagggtgaaagactcagatgatgtccccatggtgctggtgggaaataaatgtgatctgccagcacggacagtggagacccggcaagcgcaggacctggcccggagttacgggatcccctacatagaaacgtcggccaaaaccagacagggcgtcgaagatgccttctataccttagtgcgggagatccgtcagcataaactgcgcaagctgaacccaccagatgagagtggccctggctgcatgaactgtaaatgcgtgatatcgtga

SEQ ID NO: 86 - DNA, 2460 bp, synthetic construct
  source 1..2460: mol_type = other DNA
atgttcacgtggagatgtctcatcctgtgggcggtactggttaccgcaacgttgagcgcagctaggcccgcccccacgctgcctgaccaggctctccctaaggcgaatatagaagtagaatcacattctgcacatccaggagacttgttgcaattgcggtgccgcttgagagacgatgtccaatcaatcaattgggtcagggacggggtccaacttccggagaataaccggacaaggattactggagaggaggttgaagtcagggacgctgttcccgaagatagcggcttgtacgcttgcatgactaattccccgtcagggtcagaaaccacgtatttttctgtaaacgttagcgatgctcttccgagtgctgaagacgatgatgatgaagacgatagcagctccgaagagaaagaggcggataatactaaaccaaatcaagctgttgcaccatattggacctaccccgaaaaaatggagaaaaagttgcatgctgttccggcagccaaaaccgtaaaattcaagtgcccctccggcggtacccctaatccaactctgagatggttgaagaacggtaaggagttcaaaccggatcaccggataggtggatataaagttcggtatgcgacctggtccattattatggactctgtcgtgccctccgacaaaggtaactacacttgtatcgtcgagaacaagtacggcagcatcaatcatacgtaccaactggacgtggtagaacgcagtccacaccgccccatcctccaggccggactccctgccaacaaaacagttgcgctcggctctaatgttgaatttgtgtgcaaagtttactcagaccctcaacctcatatccaatggcttaaacatatcgaagtcaacggtagtaagataggtcccgacaacctgccgtatgtccagatccttaaaactgcgggggtaaataccactgacaaggaaatggaggtcttgcatcttcgcaacgtgagctttgaagatgcaggtgagtatacttgtttggcaggtaatagcatcggaatctcccaccattccgcctggctgacagtgctggaggccacggaacaaagtccggccatgatgaccagtccgttgtatctggaaattatcatttactgtacaggggcctttctcatatcatgcatggtcgtaaccgtgattatatataaaatgaagagcacaacgaagaagactgattttaattcacaactggcggtacataaactcgcaaaatctatcccattgaggcggcaggttacagtctccgccgacagctccagcagcatgaactcaggagtgatgcttgttcgccccagcagactgagttctagtgggactccgatgcttgctggagtcagtgaatacgaattgccggaggatccgcggtgggaacttcctagggaccgccttatattgggcaaacccctcggtgagggctgtttcggacaggtcgtgctcgcagaggccatcggccttgacaaagataagccgaatagagtgaccaaggtggcggttaaaatgctgaaatcagacgctacggaaaaggacctctcagacctcatcagtgaaatggaaatgatgaaaatgatagggaagcacaaaaacatcatcaatttgctcggagcttgtacccaggacggtcccctctacgtgatcgtagaatacgcttccaaaggtaatctgcgcgaatatctgcaagctcggaggccgccaggtatggaatactgttataatccgacacggattcccgaggaacagctctcttttaaagatttggtttcatgcgcgtatcaagtggcgaggggcatggagtatctggcgtccaagaagtgcattcatagggacttggctgcaagaaatgtcttggtaacagaagacaacgtcatgaagatcgccgacttcggccttgcacgggatattcatcacatcgactattacaaaaagacgacgaacggccgcctcccagttaagtggatggcccccgaagccctgttcgatcggatttacacgcatcaatccgacgtgtggtctttcggtgtcctgctttgggagatatttacactcgggggatcaccctaccccggagtaccggtggaggagttgttcaaacttcttaaagaaggtcacagaatggacaaacccagtaactgcactaacgagctgtatatgatgatgcgcgattgctggcacgctgttccgtcacaacggcccacttttaaacagctcgtggaggatcttgacagaatcgtcgcgatgactagcaaccaagagtatttggatttgtcagtcccgcttgaccaatattcccccggttttccggctacccgctcttctacttgttccagcggtgaggatagtgtattttctcatgacccacttccagatgagccgtgcttgcctcggtgtcctccccactcccatggagcgctcaaacgccactga

SEQ ID NO: 87 - DNA, 2460 bp, synthetic construct
  source 1..2460: mol_type = other DNA
atgttcacgtggagatgtctcatcctgtgggcggtactggttaccgcaacgttgagcgcagctaggcccgcccccacgctgcctgaccaggctctccctaaggcgaatatagaagtagaatcacattctgcacatccaggagacttgttgcaattgcggtgccgcttgagagacgatgtccaatcaatcaattgggtcagggacggggtccaacttccggagaataaccggacaaggattactggagaggaggttgaagtcagggacgctgttcccgaagatagcggcttgtacgcttgcatgactaattccccgtcagggtcagaaaccacgtatttttctgtaaacgttagcgatgctcttccgagtgctgaagacgatgatgatgaagacgatagcagctccgaagagaaagaggcggataatactaaaccaaatcaagctgttgcaccatattggacctaccccgaaaaaatggagaaaaagttgcatgctgttccggcagccaaaaccgtaaaattcaagtgcccctccggcggtacccctaatccaactctgagatggttgaagaacggtaaggagttcaaaccggatcaccggataggtggatataaagttcggtatgcgacctggtccattattatggactctgtcgtgccctccgacaaaggtaactacacttgtatcgtcgagaacaagtacggcagcatcaatcatacgtaccaactggacgtggtagaacgcagtccacaccgccccatcctccaggccggactccctgccaacaaaacagttgcgctcggctctaatgttgaatttgtgtgcaaagtttactcagaccctcaacctcatatccaatggcttaaacatatcgaagtcaacggtagtaagataggtcccgacaacctgccgtatgtccagatccttaaaactgcgggggtaaataccactgacaaggaaatggaggtcttgcatcttcgcaacgtgagctttgaagatgcaggtgagtatacttgtttggcaggtaatagcatcggaatctcccaccattccgcctggctgacagtgctggaggccacggaacaaagtccggccatgatgaccagtccgttgtatctggaaattatcatttactgtacaggggcctttctcatatcatgcatggtcgtaaccgtgattatatataaaatgaagagcacaacgaagaagactgattttaattcacaactggcggtacataaactcgcaaaatctatcccattgaggcggcaggttacagtctccgccgacagctccagcagcatgaactcaggagtgatgcttgttcgccccagcagactgagttctagtgggactccgatgcttgctggagtcagtgaatacgaattgccggaggatccgcggtgggaacttcctagggaccgccttatattgggcaaacccctcggtgagggctgtttcggacaggtcgtgctcgcagaggccatcggccttgacaaagataagccgaatagagtgaccaaggtggcggttaaaatgctgaaatcagacgctacggaaaaggacctctcagacctcatcagtgaaatggaaatgatgaaaatgatagggaagcacaaaaacatcatcaagttgctcggagcttgtacccaggacggtcccctctacgtgatcgtagaatacgcttccaaaggtaatctgcgcgaatatctgcaagctcggaggccgccaggtatggaatactgttataatccgacacggattcccgaggaacagctctcttttaaagatttggtttcatgcgcgtatcaagtggcgaggggcatggagtatctggcgtccaagaagtgcattcatagggacttggctgcaagaaatgtcttggtaacagaagacaacgtcatgaagatcgccgacttcggccttgcacgggatattcatcacatcgactattacaaaaagacgacgaacggccgcctcccagttaagtggatggcccccgaagccctgttcgatcggatttacacgcatcaatccgacgtgtggtctttcggtgtcctgctttgggagatatttacactcgggggatcaccctaccccggagtaccggtggaggagttgttcaaacttcttaaagaaggtcacagaatggacaaacccagtaactgcactaacgagctgtatatgatgatgcgcgattgctggcacgctgttccgtcacaacggcccacttttaaacagctcgtggaggatcttgacagaatcgtcgcgatgactagcaaccaagagtatttggatttgtcagtcccgcttgaccaatattcccccggttttccggctacccgctcttctacttgttccagcggtgaggatagtgtattttctcatgacccacttccagatgagccgtgcttgcctcggtgtcctccccactcccatggagcgctcaaacgccactga

SEQ ID NO: 88 - DNA, 2469 bp, synthetic construct
  source 1..2469: mol_type = other DNA
gccgccaccatgttcacgtggagatgtctcatcctgtgggcggtactggttaccgcaacgttgagcgcagctaggcccgcccccacgctgcctgaccaggctctccctaaggcgaatatagaagtagaatcacattctgcacatccaggagacttgttgcaattgcggtgccgcttgagagacgatgtccaatcaatcaattgggtcagggacggggtccaacttccggagaataaccggacaaggattactggagaggaggttgaagtcagggacgctgttcccgaagatagcggcttgtacgcttgcatgactaattccccgtcagggtcagaaaccacgtatttttctgtaaacgttagcgatgctcttccgagtgctgaagacgatgatgatgaagacgatagcagctccgaagagaaagaggcggataatactaaaccaaatcaagctgttgcaccatattggacctaccccgaaaaaatggagaaaaagttgcatgctgttccggcagccaaaaccgtaaaattcaagtgcccctccggcggtacccctaatccaactctgagatggttgaagaacggtaaggagttcaaaccggatcaccggataggtggatataaagttcggtatgcgacctggtccattattatggactctgtcgtgccctccgacaaaggtaactacacttgtatcgtcgagaacaagtacggcagcatcaatcatacgtaccaactggacgtggtagaacgcagtccacaccgccccatcctccaggccggactccctgccaacaaaacagttgcgctcggctctaatgttgaatttgtgtgcaaagtttactcagaccctcaacctcatatccaatggcttaaacatatcgaagtcaacggtagtaagataggtcccgacaacctgccgtatgtccagatccttaaaactgcgggggtaaataccactgacaaggaaatggaggtcttgcatcttcgcaacgtgagctttgaagatgcaggtgagtatacttgtttggcaggtaatagcatcggaatctcccaccattccgcctggctgacagtgctggaggccacggaacaaagtccggccatgatgaccagtccgttgtatctggaaattatcatttactgtacaggggcctttctcatatcatgcatggtcgtaaccgtgattatatataaaatgaagagcacaacgaagaagactgattttaattcacaactggcggtacataaactcgcaaaatctatcccattgaggcggcaggttacagtctccgccgacagctccagcagcatgaactcaggagtgatgcttgttcgccccagcagactgagttctagtgggactccgatgcttgctggagtcagtgaatacgaattgccggaggatccgcggtgggaacttcctagggaccgccttatattgggcaaacccctcggtgagggctgtttcggacaggtcgtgctcgcagaggccatcggccttgacaaagataagccgaatagagtgaccaaggtggcggttaaaatgctgaaatcagacgctacggaaaaggacctctcagacctcatcagtgaaatggaaatgatgaaaatgatagggaagcacaaaaacatcatcaatttgctcggagcttgtacccaggacggtcccctctacgtgatcatggaatacgcttccaaaggtaatctgcgcgaatatctgcaagctcggaggccgccaggtatggaatactgttataatccgacacggattcccgaggaacagctctcttttaaagatttggtttcatgcgcgtatcaagtggcgaggggcatggagtatctggcgtccaagaagtgcattcatagggacttggctgcaagaaatgtcttggtaacagaagacaacgtcatgaagatcgccgacttcggccttgcacgggatattcatcacatcgactattacaaaaagacgacgaacggccgcctcccagttaagtggatggcccccgaagccctgttcgatcggatttacacgcatcaatccgacgtgtggtctttcggtgtcctgctttgggagatatttacactcgggggatcaccctaccccggagtaccggtggaggagttgttcaaacttcttaaagaaggtcacagaatggacaaacccagtaactgcactaacgagctgtatatgatgatgcgcgattgctggcacgctgttccgtcacaacggcccacttttaaacagctcgtggaggatcttgacagaatcgtcgcgatgactagcaaccaagagtatttggatttgtcagtcccgcttgaccaatattcccccggttttccggctacccgctcttctacttgttccagcggtgaggatagtgtattttctcatgacccacttccagatgagccgtgcttgcctcggtgtcctccccactcccatggagcgctcaaacgccactga

SEQ ID NO: 89 - DNA, 2460 bp, synthetic construct
  source 1..2460: mol_type = other DNA
atgttcacgtggagatgtctcatcctgtgggcggtactggttaccgcaacgttgagcgcagctaggcccgcccccacgctgcctgaccaggctctccctaaggcgaatatagaagtagaatcacattctgcacatccaggagacttgttgcaattgcggtgccgcttgagagacgatgtccaatcaatcaattgggtcagggacggggtccaacttccggagaataaccggacaaggattactggagaggaggttgaagtcagggacgctgttcccgaagatagcggcttgtacgcttgcatgactaattccccgtcagggtcagaaaccacgtatttttctgtaaacgttagcgatgctcttccgagtgctgaagacgatgatgatgaagacgatagcagctccgaagagaaagaggcggataatactaaaccaaatcaagctgttgcaccatattggacctaccccgaaaaaatggagaaaaagttgcatgctgttccggcagccaaaaccgtaaaattcaagtgcccctccggcggtacccctaatccaactctgagatggttgaagaacggtaaggagttcaaaccggatcaccggataggtggatataaagttcggtatgcgacctggtccattattatggactctgtcgtgccctccgacaaaggtaactacacttgtatcgtcgagaacaagtacggcagcatcaatcatacgtaccaactggacgtggtagaacgcagtccacaccgccccatcctccaggccggactccctgccaacaaaacagttgcgctcggctctaatgttgaatttgtgtgcaaagtttactcagaccctcaacctcatatccaatggcttaaacatatcgaagtcaacggtagtaagataggtcccgacaacctgccgtatgtccagatccttaaaactgcgggggtaaataccactgacaaggaaatggaggtcttgcatcttcgcaacgtgagctttgaagatgcaggtgagtatacttgtttggcaggtaatagcatcggaatctcccaccattccgcctggctgacagtgctggaggccacggaacaaagtccggccatgatgaccagtccgttgtatctggaaattatcatttactgtacaggggcctttctcatatcatgcatggtcgtaaccgtgattatatataaaatgaagagcacaacgaagaagactgattttaattcacaactggcggtacataaactcgcaaaatctatcccattgaggcggcaggttacagtctccgccgacagctccagcagcatgaactcaggagtgatgcttgttcgccccagcagactgagttctagtgggactccgatgcttgctggagtcagtgaatacgaattgccggaggatccgcggtgggaacttcctagggaccgccttatattgggcaaacccctcggtgagggctgtttcggacaggtcgtgctcgcagaggccatcggccttgacaaagataagccgaatagagtgaccaaggtggcggttaaaatgctgaaatcagacgctacggaaaaggacctctcagacctcatcagtgaaatggaaatgatgaaaatgatagggaagcacaaaaacatcatcaatttgctcggagcttgtacccaggacggtcccctctacgtgatcgtagaatacgcttccaaaggtaatctgcgcgaatatctgcaagctcggaggccgccaggtatggaatactgttataatccgacacggattcccgaggaacagctctcttttaaagatttggtttcatgcgcgtatcaagtggcgaggggcatggagtatctggcgtccaagaagtgcattcatagggacttggctgcaagaaatgtcttggtaacagaagacaacgtcatgaagatcgccgacttcggccttgcacgggatattcatcacatcgactattacaaagagacgacgaacggccgcctcccagttaagtggatggcccccgaagccctgttcgatcggatttacacgcatcaatccgacgtgtggtctttcggtgtcctgctttgggagatatttacactcgggggatcaccctaccccggagtaccggtggaggagttgttcaaacttcttaaagaaggtcacagaatggacaaacccagtaactgcactaacgagctgtatatgatgatgcgcgattgctggcacgctgttccgtcacaacggcccacttttaaacagctcgtggaggatcttgacagaatcgtcgcgatgactagcaaccaagagtatttggatttgtcagtcccgcttgaccaatattcccccggttttccggctacccgctcttctacttgttccagcggtgaggatagtgtattttctcatgacccacttccagatgagccgtgcttgcctcggtgtcctccccactcccatggagcgctcaaacgccactga

SEQ ID NO: 90 - DNA, 2460 bp, synthetic construct
  source 1..2460: mol_type = other DNA
atgttcacgtggagatgtctcatcctgtgggcggtactggttaccgcaacgttgagcgcagctaggcccgcccccacgctgcctgaccaggctctccctaaggcgaatatagaagtagaatcacattctgcacatccaggagacttgttgcaattgcggtgccgcttgagagacgatgtccaatcaatcaattgggtcagggacggggtccaacttccggagaataaccggacaaggattactggagaggaggttgaagtcagggacgctgttcccgaagatagcggcttgtacgcttgcatgactaattccccgtcagggtcagaaaccacgtatttttctgtaaacgttagcgatgctcttccgagtgctgaagacgatgatgatgaagacgatagcagctccgaagagaaagaggcggataatactaaaccaaatcaagctgttgcaccatattggacctaccccgaaaaaatggagaaaaagttgcatgctgttccggcagccaaaaccgtaaaattcaagtgcccctccggcggtactcctaatccaactctgagatggttgaagaacggtaaggagttcaaaccggatcaccggataggtggatataaagttcggtatgcgacctggtccattattatggactctgtcgtgccctccgacaaaggtaactacacttgtatcgtcgagaacaagtacggcagcatcaatcatacgtaccaactggacgtggtagaacgcagtccacaccgccccatcctccaggccggactccctgccaacaaaacagttgcgctcggctctaatgttgaatttgtgtgcaaagtttactcagaccctcaacctcatatccaatggcttaaacatatcgaagtcaacggtagtaagataggtcccgacaacctgccgtatgtccagatccttaaaactgcgggggtaaataccactgacaaggaaatggaggtcttgcatcttcgcaacgtgagctttgaagatgcaggtgagtatacttgtttggcaggtaatagcatcggaatctcccaccattccgcctggctgacagtgctggaggccacggaacaaagtccggccatgatgaccagtccgttgtatctggaaattatcatttactgtacaggggcctttctcatatcatgcatggtcgtaaccgtgattatatataaaatgaagagcacaacgaagaagactgattttaattcacaactggcggtacataaactcgcaaaatctatcccattgaggcggcaggttacagtctccgccgacagctccagcagcatgaactcaggagtgatgcttgttcgccccagcagactgagttctagtgggactccgatgcttgctggagtcagtgaatacgaattgccggaggatccgcggtgggaacttcctagggaccgccttatattgggcaaacccctcggtgagggctgtttcggacaggtcgtgctcgcagaggccatcggccttgacaaagataagccgaatagagtgaccaaggtggcggttaaaatgctgaaatcagacgctacggaaaaggacctctcagacctcatcagtgaaatggaaatgatgaaaatgatagggaagcacaaaaacatcatcaagttgctcggagcttgtacccaggacggtcccctctacgtgatcgtagaatacgcttccaaaggtaatctgcgcgaatatctgcaagctcggaggccgccaggtatggaatactgttataatccgacacggattcccgaggaacagctctcttttaaagatttggtttcatgcgcgtatcaagtggcgaggggcatggagtatctggcgtccaagaagtgcattcatagggacttggctgcaagaaatgtcttggtaacagaagacaacgtcatgaagatcgccgacttcggccttgcacgggatattcatcacatcgactattacaaagagacgacgaacggccgcctcccagttaagtggatggcccccgaagccctgttcgatcggatttacacgcatcaatccgacgtgtggtctttcggtgtcctgctttgggagatatttacactcgggggatcaccctaccccggagtaccggtggaggagttgttcaaacttcttaaagaaggtcacagaatggacaaacccagtaactgcactaacgagctgtatatgatgatgcgcgattgctggcacgctgttccgtcacaacggcccacttttaaacagctcgtggaggatcttgacagaatcgtcgcgatgactagcaaccaagagtatttggatttgtcagtcccgcttgaccaatatgcccccggttttccggctacccgctcttctacttgttccagcggtgaggatagtgtattttctcatgacccacttccagatgagccgtgcttgcctcggtgtcctccccactcccatggagcgctcaaacgccactga

SEQ ID NO: 91 - DNA, 1317 bp, synthetic construct
  source 1..1317: mol_type = other DNA
atgggatcatccaagtcaaaaccgaaagacccgtcacagagaaaaatgaagagcacaacgaagaagactgattttaattcacaactggcggtacataaactcgcaaaatctatcccattgaggcggcaggttacagtctccgccgacagctccagcagcatgaactcaggagtgatgcttgttcgccccagcagactgagttctagtgggactccgatgcttgctggagtcagtgaatacgaattgccggaggatccgcggtgggaacttcctagggaccgccttatattgggcaaacccctcggtgagggctgtttcggacaggtcgtgctcgcagaggccatcggccttgacaaagataagccgaatagagtgaccaaggtggcggttaaaatgctgaaatcagacgctacggaaaaggacctctcagacctcatcagtgaaatggaaatgatgaaaatgatagggaagcacaaaaacatcatcaatttgctcggagcttgtacccaggacggtcccctctacgtgatcgtagaatacgcttccaaaggtaatctgcgcgaatatctgcaagctcggaggccgccaggtatggaatactgttataatccgacacggattcccgaggaacagctctcttttaaagatttggtttcatgcgcgtatcaagtggcgaggggcatggagtatctggcgtccaagaagtgcattcatagggacttggctgcaagaaatgtcttggtaacagaagacaacgtcatgaagatcgccgacttcggccttgcacgggatattcatcacatcgactattacaaagagacgacgaacggccgcctcccagttaagtggatggcccccgaagccctgttcgatcggatttacacgcatcaatccgacgtgtggtctttcggtgtcctgctttgggagatatttacactcgggggatcaccctaccccggagtaccggtggaggagttgttcaaacttcttaaagaaggtcacagaatggacaaacccagtaactgcactaacgagctgtatatgatgatgcgcgattgctggcacgctgttccgtcacaacggcccacttttaaacagctcgtggaggatcttgacagaatcgtcgcgatgactagcaaccaagagtatttggatttgtcagtcccgcttgaccaatattcccccggttttccggctacccgctcttctacttgttccagcggtgaggatagtgtattttctcatgacccacttccagatgagccgtgcttgcctcggtgtcctccccactcccatggagcgctcaaacgccactga

SEQ ID NO: 92 - DNA, 2523 bp, synthetic construct
  source 1..2523: mol_type = other DNA
atgggccttaagtcaacttggagatacggcaatggcccgggtacgtactccaagaaaatggtatcttgggattccggttgtctcatttgtctggtagtggttaccatggcgggcctgagtctggcgagaccctcttttaatctggtagttgaagacgctaccttggagccagaagagccgcccactaagtatcagataagtcagcctgatgtgcactccgcgcttccgggagaacctctcgagttgcgctgtcaactcaaggatgccgtgatgatcagctggacgaaagatggagtgcctcttggaccagataatcgcactgttatcattggtgaatacttgcaaattaaagatgcatcaccacgggattctggcctttacgcgtgcacagctatcaggacgctcgactccgatacactctattttatagtcaacgttaccgatgcgttgtcaagcggggatgatgaggacgacaacgacgggtcagaagacttcgttaacgattccaatcagatgagagctccctattggacccacactgacaaaatggaaaaaaggctccacgctgtcccggctgcgaatactgtgaagtttagatgtcccgcaatgggcaaccccacgccaacaatgcggtggttgaaaaatggtaaagagttcaaacaggagcatcggataggtgggtataaagtgagaaatcaacattggagcttgatcatggagtctgtagttccttcagataaaggaaattatacatgcatcgtcgagaatcaatacggctctattaaccatacatatcacctggacgtcgtagagaggagtccccacaggcctatcctgcaagcgggactgccggcaaatgcttctgcagtcgtgggcggtgacgttgagttcgtctgtaaggtgtacagcgacgcacaaccacacatccaatggataaagcatgttgagagaaatggttcaaaatatggcccggacggcttgccctaccttcaggtgctcaaagctgcgggagtcaacactactgataaggaaatcgaggtgctctatattaggaacgttacctttgaagacgccggcgaatacacctgtctcgcgggaaactctatcggtatctcatttcacaccgcatggttgactgtgcttccagctccggagaaagagaaggagtttccgacctcccctgattacctcgaaatagcgatctattgcatcggagtttttctcatcgcgtgcatggttcttactgtgatactttgtagaatgaagaacaccaccaagaaaccggatttttcctcccaaccggccgtccacaagttgacgaaacggatcccattgaggcgccaggtgagtgctgacagctcaagttcaatgaacagtaacacgccgctcgtgaggatcactactcggctgagctctaccgcggatgcgccaatgttggcaggggtcagtgagtacgaactcccggaagatccgaagtgggagtttccgcgcgacaaactcactcttggaaaaccgcttggagagggatgcttcggacaggtcgtaatggccgaggcggttggtatagacaaagatagacccaaagaagctgtcacagtagctgtaaaaatgcttaaggatgatgccactgaaaaagatttgagcgacctcgtaagcgagatggaaatgatgaaaatgatagggaagcacaaaaatataattaacctcctgggggcctgcacacaggacggcccgttgtatgtcatcgtcgaatatgcctccaaagggaacctcagggagtaccttagagcgcgcagaccgccggggatggagtattcatttgacatcaatcgggtccccgaagaacaaatgactttcaaagaccttgtctcctgtacctatcaactcgcccgcggaatggaatatttggctagtcaaaaatgcattcaccgcgatcttgctgcacggaacgtactcgtcactgagaataacgttatgaaaatagcggatttcggcctcgcaagggacataaacaacatcgactactacaaaaaaaccacgaatggcagactgccagtcaagtggatggcgccagaagccctttttgatagagtctacacgcaccagtcagacgtgtggtcctttggagtgctcatgtgggaaatctttacgctgggtggtagcccttacccggggattcccgtggaagaacttttcaagctgttgaaagagggccatcggatggacaaacccgcaaattgcacaaatgaattgtatatgatgatgcgcgactgttggcaagccgtgccttcacagagacctacattcaagcagttggtcgaagacctcgaccggatcctgacgcttacaacgaacgaagaatacctggacttgtctggtcccttggagcaatactcaccaagctaccccgatactcggtcatcttgctctagtggcgacgatagtgtcttttcacctgatccaatgccctacgaaccgtgtctgccaaagtaccaacacatgaacggttcagtaaagacctga

SEQ ID NO: 93 - DNA, 2523 bp, synthetic construct
  source 1..2523: mol_type = other DNA
atgggccttaagtcaacttggagatacggcaatggcccgggtacgtactccaagaaaatggtatcttgggattccggttgtctcatttgtctggtagtggttaccatggcgggcctgagtctggcgagaccctcttttaatctggtagttgaagacgctaccttggagccagaagagccgcccactaagtatcagataagtcagcctgatgtgcactccgcgcttccgggagaacctctcgagttgcgctgtcaactcaaggatgccgtgatgatcagctggacgaaagatggagtgcctcttggaccagataatcgcactgttatcattggtgaatacttgcaaattaaagatgcatcaccacgggattctggcctttacgcgtgcacagctatcaggacgctcgactccgatacactctattttatagtcaacgttaccgatgcgttgtcaagcggggatgatgaggacgacaacgacgggtcagaagacttcgttaacgattccaatcagatgagagctccctattggacccacactgacaaaatggaaaaaaggctccacgctgtcccggctgcgaatactgtgaagtttagatgtcccgcaatgggcaaccccacgccaacaatgcggtggttgaaaaatggtaaagagttcaaacaggagcatcggataggtgggtataaagtgagaaatcaacattggagcttgatcatggagtctgtagttccttcagataaaggaaattatacatgcatcgtcgagaatcaatacggctctattaaccatacatatcacctggacgtcgtagagaggagtccccacaggcctatcctgcaagcgggactgccggcaaatgcttctgcagtcgtgggcggtgacgttgagttcgtctgtaaggtgtacagcgacgcacaaccacacatccaatggataaagcatgttgagagaaatggttcaaaatatggcccggacggcttgccctaccttcaggtgctcaaagctgcgggagtcaacactactgataaggaaatcgaggtgctctatattaggaacgttacctttgaagacgccggcgaatacacctgtctcgcgggaaactctatcggtatctcatttcacaccgcatggttgactgtgcttccagctccggagaaagagaaggagtttccgacctcccctgattacctcgaaatagcgatctattgcatcggagtttttctcatcgcgtgcatggttcttactgtgatactttgtagaatgaagaacaccaccaagaaaccggatttttcctcccaaccggccgtccacaagttgacgaaacggatcccattgaggcgccaggtgagtgctgacagctcaagttcaatgaacagtaacacgccgctcgtgaggatcactactcggctgagctctaccgcggatgcgccaatgttggcaggggtcagtgagtacgaactcccggaagatccgaagtgggagtttccgcgcgacaaactcactcttggaaaaccgcttggagagggatgcttcggacaggtcgtaatggccgaggcggttggtatagacaaagatagacccaaagaagctgtcacagtagctgtaaaaatgcttaaggatgatgccactgaaaaagatttgagcgacctcgtaagcgagatggaaatgatgaaaatgatagggaagcacaaaaatataattaagctcctgggggcctgcacacaggacggcccgttgtatgtcatcgtcgaatatgcctccaaagggaacctcagggagtaccttagagcgcgcagaccgccggggatggagtattcatttgacatcaatcgggtccccgaagaacaaatgactttcaaagaccttgtctcctgtacctatcaactcgcccgcggaatggaatatttggctagtcaaaaatgcattcaccgcgatcttgctgcacggaacgtactcgtcactgagaataacgttatgaaaatagcggatttcggcctcgcaagggacataaacaacatcgactactacaaaaaaaccacgaatggcagactgccagtcaagtggatggcgccagaagccctttttgatagagtctacacgcaccagtcagacgtgtggtcctttggagtgctcatgtgggaaatctttacgctgggtggtagcccttacccggggattcccgtggaagaacttttcaagctgttgaaagagggccatcggatggacaaacccgcaaattgcacaaatgaattgtatatgatgatgcgcgactgttggcaagccgtgccttcacagagacctacattcaagcagttggtcgaagacctcgaccggatcctgacgcttacaacgaacgaagaatacctggacttgtctggtcccttggagcaatactcaccaagctaccccgatactcggtcatcttgctctagtggcgacgatagtgtcttttcacctgatccaatgccctacgaaccgtgtctgccaaagtaccaacacatgaacggttcagtaaagacctga

SEQ ID NO: 94 - DNA, 2523 bp, synthetic construct
  source 1..2523: mol_type = other DNA
atgggccttaagtcaacttggagatacggcaatggcccgggtacgtactccaagaaaatggtatcttgggattccggttgtctcatttgtctggtagtggttaccatggcgggcctgagtctggcgagaccctcttttaatctggtagttgaagacgctaccttggagccagaagagccgcccactaagtatcagataagtcagcctgatgtgcactccgcgcttccgggagaacctctcgagttgcgctgtcaactcaaggatgccgtgatgatcagctggacgaaagatggagtgcctcttggaccagataatcgcactgttatcattggtgaatacttgcaaattaaagatgcatcaccacgggattctggcctttacgcgtgcacagctatcaggacgctcgactccgatacactctattttatagtcaacgttaccgatgcgttgtcaagcggggatgatgaggacgacaacgacgggtcagaagacttcgttaacgattccaatcagatgagagctccctattggacccacactgacaaaatggaaaaaaggctccacgctgtcccggctgcgaatactgtgaagtttagatgtcccgcaatgggcaaccccacgccaacaatgcggtggttgaaaaatggtaaagagttcaaacaggagcatcggataggtgggtataaagtgagaaatcaacattggagcttgatcatggagtctgtagttccttcagataaaggaaattatacatgcatcgtcgagaatcaatacggctctattaaccatacatatcacctggacgtcgtagagaggagtccccacaggcctatcctgcaagcgggactgccggcaaatgcttctgcagtcgtgggcggtgacgttgagttcgtctgtaaggtgtacagcgacgcacaaccacacatccaatggataaagcatgttgagagaaatggttcaaaatatggcccggacggcttgccctaccttcaggtgctcaaagctgcgggagtcaacactactgataaggaaatcgaggtgctctatattaggaacgttacctttgaagacgccggcgaatacacctgtctcgcgggaaactctatcggtatctcatttcacaccgcatggttgactgtgcttccagctccggagaaagagaaggagtttccgacctcccctgattacctcgaaatagcgatctattgcatcggagtttttctcatcgcgtgcatggttcttactgtgatactttgtagaatgaagaacaccaccaagaaaccggatttttcctcccaaccggccgtccacaagttgacgaaacggatcccattgaggcgccaggtgagtgctgacagctcaagttcaatgaacagtaacacgccgctcgtgaggatcactactcggctgagctctaccgcggatgcgccaatgttggcaggggtcagtgagtacgaactcccggaagatccgaagtgggagtttccgcgcgacaaactcactcttggaaaaccgcttggagagggatgcttcggacaggtcgtaatggccgaggcggttggtatagacaaagatagacccaaagaagctgtcacagtagctgtaaaaatgcttaaggatgatgccactgaaaaagatttgagcgacctcgtaagcgagatggaaatgatgaaaatgatagggaagcacaaaaatataattaacctcctgggggcctgcacacaggacggcccgttgtatgtcatcgtcgaatatgcctccaaagggaacctcagggagtaccttagagcgcgcagaccgccggggatggagtattcatttgacatcaatcgggtccccgaagaacaaatgactttcaaagaccttgtctcctgtacctatcaactcgcccgcggaatggaatatttggctagtcaaaaatgcattcaccgcgatcttgctgcacggaacgtactcgtcactgagaataacgttatgaaaatagcggatttcggcctcgcaagggacataaacaacatcgactactacaaagaaaccacgaatggcagactgccagtcaagtggatggcgccagaagccctttttgatagagtctacacgcaccagtcagacgtgtggtcctttggagtgctcatgtgggaaatctttacgctgggtggtagcccttacccggggattcccgtggaagaacttttcaagctgttgaaagagggccatcggatggacaaacccgcaaattgcacaaatgaattgtatatgatgatgcgcgactgttggcaagccgtgccttcacagagacctacattcaagcagttggtcgaagacctcgaccggatcctgacgcttacaacgaacgaagaatacctggacttgtctggtcccttggagcaatactcaccaagctaccccgatactcggtcatcttgctctagtggcgacgatagtgtcttttcacctgatccaatgccctacgaaccgtgtctgccaaagtaccaacacatgaacggttcagtaaagacctga

SEQ ID NO: 95 - DNA, 2523 bp, synthetic construct
  source 1..2523: mol_type = other DNA
atgggccttaagtcaacttggagatacggcaatggcccgggtacgtactccaagaaaatggtatcttgggattccggttgtctcatttgtctggtagtggttaccatggcgggcctgagtctggcgagaccctcttttaatctggtagttgaagacgctaccttggagccagaagagccgcccactaagtatcagataagtcagcctgatgtgcactccgcgcttccgggagaacctctcgagttgcgctgtcaactcaaggatgccgtgatgatcagctggacgaaagatggagtgcctcttggaccagataatcgcactgttatcattggtgaatacttgcaaattaaagatgcatcaccacgggattctggcctttacgcgtgcacagctatcaggacgctcgactccgatacactctattttatagtcaacgttaccgatgcgttgtcaagcggggatgatgaggacgacaacgacgggtcagaagacttcgttaacgattccaatcagatgagagctccctattggacccacactgacaaaatggaaaaaaggctccacgctgtcccggctgcgaatactgtgaagtttagatgtcccgcaatgggcaaccccacgccaacaatgcggtggttgaaaaatggtaaagagttcaaacaggagcatcggataggtgggtataaagtgagaaatcaacattggagcttgatcatggagtctgtagttccttcagataaaggaaattatacatgcatcgtcgagaatcaatacggctctattaaccatacatatcacctggacgtcgtagagaggagtccccacaggcctatcctgcaagcgggactgccggcaaatgcttctgcagtcgtgggcggtgacgttgagttcgtctgtaaggtgtacagcgacgcacaaccacacatccaatggataaagcatgttgagagaaatggttcaaaatatggcccggacggcttgccctaccttcaggtgctcaaagctgcgggagtcaacactactgataaggaaatcgaggtgctctatattaggaacgttacctttgaagacgccggcgaatacacctgtctcgcgggaaactctatcggtatctcatttcacaccgcatggttgactgtgcttccagctccggagaaagagaaggagtttccgacctcccctgattacctcgaaatagcgatctattgcatcggagtttttctcatcgcgtgcatggttcttactgtgatactttgtagaatgaagaacaccaccaagaaaccggatttttcctcccaaccggccgtccacaagttgacgaaacggatcccattgaggcgccaggtgagtgctgacagctcaagttcaatgaacagtaacacgccgctcgtgaggatcactactcggctgagctctaccgcggatgcgccaatgttggcaggggtcagtgagtacgaactcccggaagatccgaagtgggagtttccgcgcgacaaactcactcttggaaaaccgcttggagagggatgcttcggacaggtcgtaatggccgaggcggttggtatagacaaagatagacccaaagaagctgtcacagtagctgtaaaaatgcttaaggatgatgccactgaaaaagatttgagcgacctcgtaagcgagatggaaatgatgaaaatgatagggaagcacaaaaatataattaagctcctgggggcctgcacacaggacggcccgttgtatgtcatcgtcgaatatgcctccaaagggaacctcagggagtaccttagagcgcgcagaccgccggggatggagtattcatttgacatcaatcgggtccccgaagaacaaatgactttcaaagaccttgtctcctgtacctatcaactcgcccgcggaatggaatatttggctagtcaaaaatgcattcaccgcgatcttgctgcacggaacgtactcgtcactgagaataacgttatgaaaatagcggatttcggcctcgcaagggacataaacaacatcgactactacaaagaaaccacgaatggcagactgccagtcaagtggatggcgccagaagccctttttgatagagtctacacgcaccagtcagacgtgtggtcctttggagtgctcatgtgggaaatctttacgctgggtggtagcccttacccggggattcccgtggaagaacttttcaagctgttgaaagagggccatcggatggacaaacccgcaaattgcacaaatgaattgtatatgatgatgcgcgactgttggcaagccgtgccttcacagagacctacattcaagcagttggtcgaagacctcgaccggatcctgacgcttacaacgaacgaagaatacctggacttgtctggtcccttggagcaatacgcaccaagctaccccgatactcggtcatcttgctctagtggcgacgatagtgtcttttcacctgatccaatgccctacgaaccgtgtctgccaaagtaccaacacatgaacggttcagtaaagacctga

SEQ ID NO: 96 - DNA, 2439 bp, synthetic construct
  source 1..2439: mol_type = other DNA
atgtcagaagctggtggcggcgccgctgccgcagcctcactcccgagatcaagggccggtggcatgcgcgcggcatggggatccgtgtggtgtttgtgcctcgcggcggctgtcggagctctgccggctgcaaggcgcaggggagcggaacggagcggtggccaggcggcggagtatctccggagtgaaactgcatttcttgaggagcttgttttcggttcaggggataccatcgagctttcctgtaacacacaatcttcaagtgtaagcgtattctggttcaaagatggtataggcattgcgcccagtaatagaacacacatcgggcagaaacttttgaagatcatcaacgtttcatatgatgattccggactgtactcatgtaagccaaggcacagtaacgaggtgcttgggaacttcacagtcagggttacaggcgtaccattctggacgaggcccgataagatggaaaaaaaacttttggcagtgcccgcagccaatacagtgcggttccgctgccccgcaggtgggaatccgacgcccacaatttattggctgaagaatggcaaggagttcaaaggagaacataggatcggcggaatcaaactgaggcaccagcaatggtctctggtgatggaatcagtagtcccatctgaccgcggtaactacacgtgcgtagtagaaaacaagtatgggaatattcggcatacctaccagctcgatgtgctcgagagatctcctcacaggcctatcttgcaggctggactgccagctaatcaaacagtagttgtcggatctaacgtggagttccactgcaaagtttactcagacgcccaaccgcacattcaatggcttaaacacgtggaggttaacggtagcaagtatggacctgatgggactccctatgtgacagtccttaagagttggatttctaagaatgccgaggcggatgcgaacttgaacctgtttaacgtaaccgagcaggacgagggcgaatatctttgtcgcgctaacaacttcgttggaatagcggagaagcccttctggctgcatatacggaagccaaagccggcggaggagttgatggagatggatgactccggttccgtatacgccggaatccttagctacggaaccgggctggtgctgtttatcctcgttttggttattgttattatatgccgcatgaagatgccaaacaaaaaggcaatgaatactacaactgtgcagaaagtgtcaaaattcccacttaaacggcaagtcactgtctcactcgaatctaactcttccatgaactccaacactccgttggtgcggattaccagattgtcctcatccgacggtcccatgttggcaaatgtcagtgagctggaattgccgcccgatcccaaatgggaacttgcaagatcccggcttactctcggtaagcccctgggcgaaggttgctttggacaagtcgtcatggccgaagcgattggaatcgataaagacaaacccaacaaggctataacggtcgcagtaaaaatgttgaaagatgacgcgaccgacaaggacctgtcagatctggtctcagagatggaaatgatgaagatgattggtaaacacaagaatataatcaacttgcttggagcatgtacgcaagacggccctctctacgttctcgtggagtatgcctctaagggcaatttgagggagtacctgagagctcgcagacctccgggcatggattatagtttcgacacttgtaagctgccggaggagcaacttaccttcaaggatcttgtaagttgcgcgtaccaggttgcaagggggatggaatacctcgccagtcaaaagtgcatacatagagatttggcagcgaggaacgtgctcgtgactgaggacaacgtcatgaaaattgcggactttgggcttgcacgggatgttcataacatcgattactacaagaaaactactaacggacggctccctgtgaagtggatggctccagaagcgctttttgatagagtttacacacatcaaagtgacgtatggagttttggagtattgctctgggagattttcaccttgggagggtctccttaccctggaatacccgtcgaagagctctttaagctgcttaaagaaggccacaggatggacaagccggcgaactgcactcatgatctgtacatgataatgagagagtgctggcacgccgttccctcccagcggccgacattcaagcagctggtggaagaccttgatagggtactcactatgacgtccaccgatgagtatctggacttgtctgttccattcgaacaatactctccggccggtcaggatacgcattccacttgctccagtggtgatgattctgtctttgcacacgatcttctcccagacgaaccctgtcttcctaaacacgtaccgtgtaacggtgtcataaggacatag

SEQ ID NO: 97 - DNA, 2439 bp, synthetic construct
  source 1..2439: mol_type = other DNA
atgtcagaagctggtggcggcgccgctgccgcagcctcactcccgagatcaagggccggtggcatgcgcgcggcatggggatccgtgtggtgtttgtgcctcgcggcggctgtcggagctctgccggctgcaaggcgcaggggagcggaacggagcggtggccaggcggcggagtatctccggagtgaaactgcatttcttgaggagcttgttttcggttcaggggataccatcgagctttcctgtaacacacaatcttcaagtgtaagcgtattctggttcaaagatggtataggcattgcgcccagtaatagaacacacatcgggcagaaacttttgaagatcatcaacgtttcatatgatgattccggactgtactcatgtaagccaaggcacagtaacgaggtgcttgggaacttcacagtcagggttacaggcgtaccattctggacgaggcccgataagatggaaaaaaaacttttggcagtgcccgcagccaatacagtgcggttccgctgccccgcaggtgggaatccgacgcccacaatttattggctgaagaatggcaaggagttcaaaggagaacataggatcggcggaatcaaactgaggcaccagcaatggtctctggtgatggaatcagtagtcccatctgaccgcggtaactacacgtgcgtagtagaaaacaagtatgggaatattcggcatacctaccagctcgatgtgctcgagagatctcctcacaggcctatcttgcaggctggactgccagctaatcaaacagtagttgtcggatctaacgtggagttccactgcaaagtttactcagacgcccaaccgcacattcaatggcttaaacacgtggaggttaacggtagcaagtatggacctgatgggactccctatgtgacagtccttaagagttggatttctaagaatgccgaggcggatgcgaacttgaacctgtttaacgtaaccgagcaggacgagggcgaatatctttgtcgcgctaacaacttcgttggaatagcggagaagcccttctggctgcatatacggaagccaaagccggcggaggagttgatggagatggatgactccggttccgtatacgccggaatccttagctacggaaccgggctggtgctgtttatcctcgttttggttattgttattatatgccgcatgaagatgccaaacaaaaaggcaatgaatactacaactgtgcagaaagtgtcaaaattcccacttaaacggcaagtcactgtctcactcgaatctaactcttccatgaactccaacactccgttggtgcggattaccagattgtcctcatccgacggtcccatgttggcaaatgtcagtgagctggaattgccgcccgatcccaaatgggaacttgcaagatcccggcttactctcggtaagcccctgggcgaaggttgctttggacaagtcgtcatggccgaagcgattggaatcgataaagacaaacccaacaaggctataacggtcgcagtaaaaatgttgaaagatgacgcgaccgacaaggacctgtcagatctggtctcagagatggaaatgatgaagatgattggtaaacacaagaatataatcaagttgcttggagcatgtacgcaagacggccctctctacgttctcgtggagtatgcctctaagggcaatttgagggagtacctgagagctcgcagacctccgggcatggattatagtttcgacacttgtaagctgccggaggagcaacttaccttcaaggatcttgtaagttgcgcgtaccaggttgcaagggggatggaatacctcgccagtcaaaagtgcatacatagagatttggcagcgaggaacgtgctcgtgactgaggacaacgtcatgaaaattgcggactttgggcttgcacgggatgttcataacatcgattactacaagaaaactactaacggacggctccctgtgaagtggatggctccagaagcgctttttgatagagtttacacacatcaaagtgacgtatggagttttggagtattgctctgggagattttcaccttgggagggtctccttaccctggaatacccgtcgaagagctctttaagctgcttaaagaaggccacaggatggacaagccggcgaactgcactcatgatctgtacatgataatgagagagtgctggcacgccgttccctcccagcggccgacattcaagcagctggtggaagaccttgatagggtactcactatgacgtccaccgatgagtatctggacttgtctgttccattcgaacaatactctccggccggtcaggatacgcattccacttgctccagtggtgatgattctgtctttgcacacgatcttctcccagacgaaccctgtcttcctaaacacgtaccgtgtaacggtgtcataaggacatag

SEQ ID NO: 98 - DNA, 2439 bp, synthetic construct
  source 1..2439: mol_type = other DNA
atgtcagaagctggtggcggcgccgctgccgcagcctcactcccgagatcaagggccggtggcatgcgcgcggcatggggatccgtgtggtgtttgtgcctcgcggcggctgtcggagctctgccggctgcaaggcgcaggggagcggaacggagcggtggccaggcggcggagtatctccggagtgaaactgcatttcttgaggagcttgttttcggttcaggggataccatcgagctttcctgtaacacacaatcttcaagtgtaagcgtattctggttcaaagatggtataggcattgcgcccagtaatagaacacacatcgggcagaaacttttgaagatcatcaacgtttcatatgatgattccggactgtactcatgtaagccaaggcacagtaacgaggtgcttgggaacttcacagtcagggttacaggcgtaccattctggacgaggcccgataagatggaaaaaaaacttttggcagtgcccgcagccaatacagtgcggttccgctgccccgcaggtgggaatccgacgcccacaatttattggctgaagaatggcaaggagttcaaaggagaacataggatcggcggaatcaaactgaggcaccagcaatggtctctggtgatggaatcagtagtcccatctgaccgcggtaactacacgtgcgtagtagaaaacaagtatgggaatattcggcatacctaccagctcgatgtgctcgagagatctcctcacaggcctatcttgcaggctggactgccagctaatcaaacagtagttgtcggatctaacgtggagttccactgcaaagtttactcagacgcccaaccgcacattcaatggcttaaacacgtggaggttaacggtagcaagtatggacctgatgggactccctatgtgacagtccttaagagttggatttctaagaatgccgaggcggatgcgaacttgaacctgtttaacgtaaccgagcaggacgagggcgaatatctttgtcgcgctaacaacttcgttggaatagcggagaagcccttctggctgcatatacggaagccaaagccggcggaggagttgatggagatggatgactccggttccgtatacgccggaatccttagctacggaaccgggctggtgctgtttatcctcgttttggttattgttattatatgccgcatgaagatgccaaacaaaaaggcaatgaatactacaactgtgcagaaagtgtcaaaattcccacttaaacggcaagtcactgtctcactcgaatctaactcttccatgaactccaacactccgttggtgcggattaccagattgtcctcatccgacggtcccatgttggcaaatgtcagtgagctggaattgccgcccgatcccaaatgggaacttgcaagatcccggcttactctcggtaagcccctgggcgaaggttgctttggacaagtcgtcatggccgaagcgattggaatcgataaagacaaacccaacaaggctataacggtcgcagtaaaaatgttgaaagatgacgcgaccgacaaggacctgtcagatctggtctcagagatggaaatgatgaagatgattggtaaacacaagaatataatcaacttgcttggagcatgtacgcaagacggccctctctacgttctcgtggagtatgcctctaagggcaatttgagggagtacctgagagctcgcagacctccgggcatggattatagtttcgacacttgtaagctgccggaggagcaacttaccttcaaggatcttgtaagttgcgcgtaccaggttgcaagggggatggaatacctcgccagtcaaaagtgcatacatagagatttggcagcgaggaacgtgctcgtgactgaggacaacgtcatgaaaattgcggactttgggcttgcacgggatgttcataacatcgattactacaaggaaactactaacggacggctccctgtgaagtggatggctccagaagcgctttttgatagagtttacacacatcaaagtgacgtatggagttttggagtattgctctgggagattttcaccttgggagggtctccttaccctggaatacccgtcgaagagctctttaagctgcttaaagaaggccacaggatggacaagccggcgaactgcactcatgatctgtacatgataatgagagagtgctggcacgccgttccctcccagcggccgacattcaagcagctggtggaagaccttgatagggtactcactatgacgtccaccgatgagtatctggacttgtctgttccattcgaacaatactctccggccggtcaggatacgcattccacttgctccagtggtgatgattctgtctttgcacacgatcttctcccagacgaaccctgtcttcctaaacacgtaccgtgtaacggtgtcataaggacatag

SEQ ID NO: 99 - DNA, 2439 bp, synthetic construct
  source 1..2439: mol_type = other DNA
atgtcagaagctggtggcggcgccgctgccgcagcctcactcccgagatcaagggccggtggcatgcgcgcggcatggggatccgtgtggtgtttgtgcctcgcggcggctgtcggagctctgccggctgcaaggcgcaggggagcggaacggagcggtggccaggcggcggagtatctccggagtgaaactgcatttcttgaggagcttgttttcggttcaggggataccatcgagctttcctgtaacacacaatcttcaagtgtaagcgtattctggttcaaagatggtataggcattgcgcccagtaatagaacacacatcgggcagaaacttttgaagatcatcaacgtttcatatgatgattccggactgtactcatgtaagccaaggcacagtaacgaggtgcttgggaacttcacagtcagggttacaggcgtaccattctggacgaggcccgataagatggaaaaaaaacttttggcagtgcccgcagccaatacagtgcggttccgctgccccgcaggtgggaatccgacgcccacaatttattggctgaagaatggcaaggagttcaaaggagaacataggatcggcggaatcaaactgaggcaccagcaatggtctctggtgatggaatcagtagtcccatctgaccgcggtaactacacgtgcgtagtagaaaacaagtatgggaatattcggcatacctaccagctcgatgtgctcgagagatctcctcacaggcctatcttgcaggctggactgccagctaatcaaacagtagttgtcggatctaacgtggagttccactgcaaagtttactcagacgcccaaccgcacattcaatggcttaaacacgtggaggttaacggtagcaagtatggacctgatgggactccctatgtgacagtccttaagagttggatttctaagaatgccgaggcggatgcgaacttgaacctgtttaacgtaaccgagcaggacgagggcgaatatctttgtcgcgctaacaacttcgttggaatagcggagaagcccttctggctgcatatacggaagccaaagccggcggaggagttgatggagatggatgactccggttccgtatacgccggaatccttagctacggaaccgggctggtgctgtttatcctcgttttggttattgttattatatgccgcatgaagatgccaaacaaaaaggcaatgaatactacaactgtgcagaaagtgtcaaaattcccacttaaacggcaagtcactgtctcactcgaatctaactcttccatgaactccaacactccgttggtgcggattaccagattgtcctcatccgacggtcccatgttggcaaatgtcagtgagctggaattgccgcccgatcccaaatgggaacttgcaagatcccggcttactctcggtaagcccctgggcgaaggttgctttggacaagtcgtcatggccgaagcgattggaatcgataaagacaaacccaacaaggctataacggtcgcagtaaaaatgttgaaagatgacgcgaccgacaaggacctgtcagatctggtctcagagatggaaatgatgaagatgattggtaaacacaagaatataatcaagttgcttggagcatgtacgcaagacggccctctctacgttctcgtggagtatgcctctaagggcaatttgagggagtacctgagagctcgcagacctccgggcatggattatagtttcgacacttgtaagctgccggaggagcaacttaccttcaaggatcttgtaagttgcgcgtaccaggttgcaagggggatggaatacctcgccagtcaaaagtgcatacatagagatttggcagcgaggaacgtgctcgtgactgaggacaacgtcatgaaaattgcggactttgggcttgcacgggatgttcataacatcgattactacaaggaaactactaacggacggctccctgtgaagtggatggctccagaagcgctttttgatagagtttacacacatcaaagtgacgtatggagttttggagtattgctctgggagattttcaccttgggagggtctccttaccctggaatacccgtcgaagagctctttaagctgcttaaagaaggccacaggatggacaagccggcgaactgcactcatgatctgtacatgataatgagagagtgctggcacgccgttccctcccagcggccgacattcaagcagctggtggaagaccttgatagggtactcactatgacgtccaccgatgagtatctggacttgtctgttccattcgaacaatactctccggccggtcaggatacgcattccacttgctccagtggtgatgattctgtctttgcacacgatcttctcccagacgaaccctgtcttcctaaacacgtaccgtgtaacggtgtcataaggacatag

SEQ ID NO: 100 - DNA, 1299 bp, synthetic construct
  source 1..1299: mol_type = other DNA
atgggatcatccaagtcaaaaccgaaagacccgtcacagagacgcatgaagatgccaaacaaaaaggcaatgaatactacaactgtgcagaaagtgtcaaaattcccacttaaacggcaagtcactgtctcactcgaatctaactcttccatgaactccaacactccgttggtgcggattaccagattgtcctcatccgacggtcccatgttggcaaatgtcagtgagctggaattgccgcccgatcccaaatgggaacttgcaagatcccggcttactctcggtaagcccctgggcgaaggttgctttggacaagtcgtcatggccgaagcgattggaatcgataaagacaaacccaacaaggctataacggtcgcagtaaaaatgttgaaagatgacgcgaccgacaaggacctgtcagatctggtctcagagatggaaatgatgaagatgattggtaaacacaagaatataatcaacttgcttggagcatgtacgcaagacggccctctctacgttctcgtggagtatgcctctaagggcaatttgagggagtacctgagagctcgcagacctccgggcatggattatagtttcgacacttgtaagctgccggaggagcaacttaccttcaaggatcttgtaagttgcgcgtaccaggttgcaagggggatggaatacctcgccagtcaaaagtgcatacatagagatttggcagcgaggaacgtgctcgtgactgaggacaacgtcatgaaaattgcggactttgggcttgcacgggatgttcataacatcgattactacaaggaaactactaacggacggctccctgtgaagtggatggctccagaagcgctttttgatagagtttacacacatcaaagtgacgtatggagttttggagtattgctctgggagattttcaccttgggagggtctccttaccctggaatacccgtcgaagagctctttaagctgcttaaagaaggccacaggatggacaagccggcgaactgcactcatgatctgtacatgataatgagagagtgctggcacgccgttccctcccagcggccgacattcaagcagctggtggaagaccttgatagggtactcactatgacgtccaccgatgagtatctggacttgtctgttccattcgaacaatactctccggccggtcaggatacgcattccacttgctccagtggtgatgattctgtctttgcacacgatcttctcccagacgaaccctgtcttcctaaacacgtaccgtgtaacggtgtcataaggacatag

SEQ ID NO: 101 - DNA, 2409 bp, synthetic construct
  source 1..2409: mol_type = other DNA
atgcttcctctgcgcctggttctcgctggcctcttggtcgcagcgggttcagcggcgagtcataggggagaaatggagccggaactctttgagtctccactcttggaatccgaagaagaacacctccttctggacccaggaaacgcattgaaactctattgtgacgtaaaccagtccggagctagtgtggtttggtataaggagagtagacctctgctgccagggccccgcgtcagattgcaacaaagcgttcttgaaatagcggaagtagcttacgaggattccggcctctacgtctgtagagctcgcggaaccggtgaggtccttaggaacttcaccatatcagttgtagattcacttgcctcaggcgatgacgatgaagacagcgatggggatggtccacatggagaccgctctgaagaaccagtatacgttcacagagcaccttattggacccatccacacaggatggataaaaaactctacgctgttcctgcgggcaataccgtgaaattccgctgtccagcgagtgggtctccaagcccgtccattagatggtttaagaatggcagagagtttcgcggggagcacagaatagggggcattaggctccggcatcagcattggtcactcgttatggagtcagtcgtgccgtctgatagggggaattacacctgcttggtagagaaccggtttggttcaatccgctatagttatctgctggatgtcctcgaacgctccccacacagacccatcttgcaagctggacttccagctaacacaacagctctggtaggttcagatgtggaatttttctgcaaggtatactctgatgctcaaccgcacatacagtggttgaaacacattgaagttaacgggtcctcatatgggccagacggtgtaccctacgtgcaggtactgaagacggccgacattaattcatctgaggttgaggtgctgtatttgcggaacgtcacaatggaagacgccggggagtatacttgtcttgccggtaatagtattgggctgtcctatcagtccgcgtggctcaccgtcctgccagaagagctggttcatgaggctgaggcacctgaggcgaaatacaccgacatcataatttacacttccggatcattggccgtggcaatggctcttatcatcgtcgttctgtgtaggatgcaaactcagagctctaaacaaccccttgaacccatggcagtacacaaattgagcaaatttcctcttattagacagttttcccttgactcaagtagctcagggaaatctagcacatcacttatgcgggtgacgagactgtcttccagctgcgcgcccatgttggctggggtggtggaacttgatctgcctctggacagcaagtgggaatttccgagggagaaactggttcttgggaagccgctcggcgaaggctgttttggtcaggtggtcagggcggaagcgtacgggatcgacagacagtggcctgatcgcgcagttactgtcgcagtaaaaatgctgaaagacaacgctactgataaggatctggcagacctgataagtgaaatggagatgatgaaactgatggacaaacacaagaacatcattaatctcttgggtgtatgtacacaagatgggcctctgtatgttatagtagagtttgcggccaaaggcaaccttcgggagtatcttcgcgctagaagaccgccaatgcccgactacacgttcgatattacagaactccatgaggaacaactttgttttaaggatcttgttagctgtgtgtatcaagtcgcccgggggatggagtatctggaatcaagacggtgtatacaccgcgacctcgctgccagaaacgttctcgtcacggcggaaaatgtgatgaagatcgccgacttcggacttgccagggatgtccatgatatagactattacaaaaaaacatctaatgggcggctccctgtcaagtggatggcgcccgaagcactgtttgacagagtatacacgcaccagtctgacgtgtggtcatttggcatactgatgtgggaaatttttacactcggtggttcaccttatcctggcatccctgttgaggagctttttaaattgctcaaagagggccacagaatggactgtcctagtaactgcacccatgagctgtatatgctcatgcgcgagtgctggcatgcggtgcctagtcaaaggccaaccttcaaacagctcgtcgaaggcctggacaagattcttgctgcaataagcgaggagtacctcgacttgtctatgccattcgagcaatactcaccttcttgtgaagacacgacgagtacatgcagcagcgacgactctgtatttacacacgaccctttgccccttgctccttgcctgtttgcctgtcctagtggccgcacctag

SEQ ID NO: 102 - DNA, 2409 bp, synthetic construct
  source 1..2409: mol_type = other DNA
atgcttcctctgcgcctggttctcgctggcctcttggtcgcagcgggttcagcggcgagtcataggggagaaatggagccggaactctttgagtctccactcttggaatccgaagaagaacacctccttctggacccaggaaacgcattgaaactctattgtgacgtaaaccagtccggagctagtgtggtttggtataaggagagtagacctctgctgccagggccccgcgtcagattgcaacaaagcgttcttgaaatagcggaagtagcttacgaggattccggcctctacgtctgtagagctcgcggaaccggtgaggtccttaggaacttcaccatatcagttgtagattcacttgcctcaggcgatgacgatgaagacagcgatggggatggtccacatggagaccgctctgaagaaccagtatacgttcacagagcaccttattggacccatccacacaggatggataaaaaactctacgctgttcctgcgggcaataccgtgaaattccgctgtccagcgagtgggtctccaagcccgtccattagatggtttaagaatggcagagagtttcgcggggagcacagaatagggggcattaggctccggcatcagcattggtcactcgttatggagtcagtcgtgccgtctgatagggggaattacacctgcttggtagagaaccggtttggttcaatccgctatagttatctgctggatgtcctcgaacgctccccacacagacccatcttgcaagctggacttccagctaacacaacagctctggtaggttcagatgtggaatttttctgcaaggtatactctgatgctcaaccgcacatacagtggttgaaacacattgaagttaacgggtcctcatatgggccagacggtgtaccctacgtgcaggtactgaagacggccgacattaattcatctgaggttgaggtgctgtatttgcggaacgtcacaatggaagacgccggggagtatacttgtcttgccggtaatagtattgggctgtcctatcagtccgcgtggctcaccgtcctgccagaagagctggttcatgaggctgaggcacctgaggcgaaatgtaccgacatcataatttacacttccggatcattggccgtggcaatggctcttatcatcgtcgttctgtgtaggatgcaaactcagagctctaaacaaccccttgaacccatggcagtacacaaattgagcaaatttcctcttattagacagttttcccttgactcaagtagctcagggaaatctagcacatcacttatgcgggtgacgagactgtcttccagctgcgcgcccatgttggctggggtggtggaacttgatctgcctctggacagcaagtgggaatttccgagggagaaactggttcttgggaagccgctcggcgaaggctgttttggtcaggtggtcagggcggaagcgtacgggatcgacagacagtggcctgatcgcgcagttactgtcgcagtaaaaatgctgaaagacaacgctactgataaggatctggcagacctgataagtgaaatggagatgatgaaactgatggacaaacacaagaacatcattaatctcttgggtgtatgtacacaagatgggcctctgtatgttatagtagagtttgcggccaaaggcaaccttcgggagtatcttcgcgctagaagaccgccaatgcccgactacacgttcgatattacagaactccatgaggaacaactttgttttaaggatcttgttagctgtgtgtatcaagtcgcccgggggatggagtatctggaatcaagacggtgtatacaccgcgacctcgctgccagaaacgttctcgtcacggcggaaaatgtgatgaagatcgccgacttcggacttgccagggatgtccatgatatagactattacaaaaaaacatctaatgggcggctccctgtcaagtggatggcgcccgaagcactgtttgacagagtatacacgcaccagtctgacgtgtggtcatttggcatactgatgtgggaaatttttacactcggtggttcaccttatcctggcatccctgttgaggagctttttaaattgctcaaagagggccacagaatggactgtcctagtaactgcacccatgagctgtatatgctcatgcgcgagtgctggcatgcggtgcctagtcaaaggccaaccttcaaacagctcgtcgaaggcctggacaagattcttgctgcaataagcgaggagtacctcgacttgtctatgccattcgagcaatactcaccttcttgtgaagacacgacgagtacatgcagcagcgacgactctgtatttacacacgaccctttgccccttgctccttgcctgtttgcctgtcctagtggccgcacctag

SEQ ID NO: 103 - DNA, 2409 bp, synthetic construct
  source 1..2409: mol_type = other DNA
atgcttcctctgcgcctggttctcgctggcctcttggtcgcagcgggttcagcggcgagtcataggggagaaatggagccggaactctttgagtctccactcttggaatccgaagaagaacacctccttctggacccaggaaacgcattgaaactctattgtgacgtaaaccagtccggagctagtgtggtttggtataaggagagtagacctctgctgccagggccccgcgtcagattgcaacaaagcgttcttgaaatagcggaagtagcttacgaggattccggcctctacgtctgtagagctcgcggaaccggtgaggtccttaggaacttcaccatatcagttgtagattcacttgcctcaggcgatgacgatgaagacagcgatggggatggtccacatggagaccgctctgaagaaccagtatacgttcacagagcaccttattggacccatccacacaggatggataaaaaactctacgctgttcctgcgggcaataccgtgaaattccgctgtccagcgagtgggtctccaagcccgtccattagatggtttaagaatggcagagagtttcgcggggagcacagaatagggggcattaggctccggcatcagcattggtcactcgttatggagtcagtcgtgccgtctgatagggggaattacacctgcttggtagagaaccggtttggttcaatccgctatagttatctgctggatgtcctcgaacgctccccacacagacccatcttgcaagctggacttccagctaacacaacagctctggtaggttcagatgtggaatttttctgcaaggtatactctgatgctcaaccgcacatacagtggttgaaacacattgaagttaacgggtcctcatatgggccagacggtgtaccctacgtgcaggtactgaagacggccgacattaattcatctgaggttgaggtgctgtatttgcggaacgtcacaatggaagacgccggggagtatacttgtcttgccggtaatagtattgggctgtcctatcagtccgcgtggctcaccgtcctgccagaagagctggttcatgaggctgaggcacctgaggcgaaatacaccgacatcataatttacacttccggatcattggccgtggcaatggctcttatcatcgtcgttctgtgtaggatgcaaactcagagctctaaacaaccccttgaacccatggcagtacacaaattgagcaaatttcctcttattagacagttttcccttgactcaagtagctcagggaaatctagcacatcacttatgcgggtgacgagactgtcttccagctgcgcgcccatgttggctggggtggtggaacttgatctgcctctggacagcaagtgggaatttccgagggagaaactggttcttgggaagccgctcggcgaaggctgttttggtcaggtggtcagggcggaagcgtacgggatcgacagacagtggcctgatcgcgcagttactgtcgcagtaaaaatgctgaaagacaacgctactgataaggatctggcagacctgataagtgaaatggagatgatgaaactgatggacaaacacaagaacatcattaatctcttgggtgtatgtacacaagatgggcctctgtatgttatagtagagtttgcggccaaaggcaaccttcgggagtatcttcgcgctagaagaccgccaatgcccgactacacgttcgatattacagaactccatgaggaacaactttgttttaaggatcttgttagctgtgtgtatcaagtcgcccgggggatggagtatctggaatcaagacggtgtatacaccgcgacctcgctgccagaaacgttctcgtcacggcggaaaatgtgatgaagatcgccgacttcggacttgccagggatgtccatgatatagactattacaaagaaacatctaatgggcggctccctgtcaagtggatggcgcccgaagcactgtttgacagagtatacacgcaccagtctgacgtgtggtcatttggcatactgatgtgggaaatttttacactcggtggttcaccttatcctggcatccctgttgaggagctttttaaattgctcaaagagggccacagaatggactgtcctagtaactgcacccatgagctgtatatgctcatgcgcgagtgctggcatgcggtgcctagtcaaaggccaaccttcaaacagctcgtcgaaggcctggacaagattcttgctgcaataagcgaggagtacctcgacttgtctatgccattcgagcaatactcaccttcttgtgaagacacgacgagtacatgcagcagcgacgactctgtatttacacacgaccctttgccccttgctccttgcctgtttgcctgtcctagtggccgcacctag

SEQ ID NO: 104 - DNA, 2409 bp, synthetic construct
  source 1..2409: mol_type = other DNA
atgcttcctctgcgcctggttctcgctggcctcttggtcgcagcgggttcagcggcgagtcataggggagaaatggagccggaactctttgagtctccactcttggaatccgaagaagaacacctccttctggacccaggaaacgcattgaaactctattgtgacgtaaaccagtccggagctagtgtggtttggtataaggagagtagacctctgctgccagggccccgcgtcagattgcaacaaagcgttcttgaaatagcggaagtagcttacgaggattccggcctctacgtctgtagagctcgcggaaccggtgaggtccttaggaacttcaccatatcagttgtagattcacttgcctcaggcgatgacgatgaagacagcgatggggatggtccacatggagaccgctctgaagaaccagtatacgttcacagagcaccttattggacccatccacacaggatggataaaaaactctacgctgttcctgcgggcaataccgtgaaattccgctgtccagcgagtgggtctccaagcccgtccattagatggtttaagaatggcagagagtttcgcggggagcacagaatagggggcattaggctccggcatcagcattggtcactcgttatggagtcagtcgtgccgtctgatagggggaattacacctgcttggtagagaaccggtttggttcaatccgctatagttatctgctggatgtcctcgaacgctccccacacagacccatcttgcaagctggacttccagctaacacaacagctctggtaggttcagatgtggaatttttctgcaaggtatactctgatgctcaaccgcacatacagtggttgaaacacattgaagttaacgggtcctcatatgggccagacggtgtaccctacgtgcaggtactgaagacggccgacattaattcatctgaggttgaggtgctgtatttgcggaacgtcacaatggaagacgccggggagtatacttgtcttgccggtaatagtattgggctgtcctatcagtccgcgtggctcaccgtcctgccagaagagctggttcatgaggctgaggcacctgaggcgaaatgtaccgacatcataatttacacttccggatcattggccgtggcaatggctcttatcatcgtcgttctgtgtaggatgcaaactcagagctctaaacaaccccttgaacccatggcagtacacaaattgagcaaatttcctcttattagacagttttcccttgactcaagtagctcagggaaatctagcacatcacttatgcgggtgacgagactgtcttccagctgcgcgcccatgttggctggggtggtggaacttgatctgcctctggacagcaagtgggaatttccgagggagaaactggttcttgggaagccgctcggcgaaggctgttttggtcaggtggtcagggcggaagcgtacgggatcgacagacagtggcctgatcgcgcagttactgtcgcagtaaaaatgctgaaagacaacgctactgataaggatctggcagacctgataagtgaaatggagatgatgaaactgatggacaaacacaagaacatcattaatctcttgggtgtatgtacacaagatgggcctctgtatgttatagaagagtttgcggccaaaggcaaccttcgggagtatcttcgcgctagaagaccgccaatgcccgactacacgttcgatattacagaactccatgaggaacaactttgttttaaggatcttgttagctgtgtgtatcaagtcgcccgggggatggagtatctggaatcaagacggtgtatacaccgcgacctcgctgccagaaacgttctcgtcacggcggaaaatgtgatgaagatcgccgacttcggacttgccagggatgtccatgatatagactattacaaaaaaacatctaatgggcggctccctgtcaagtggatggcgcccgaagcactgtttgacagagtatacacgcaccagtctgacgtgtggtcatttggcatactgatgtgggaaatttttacactcggtggttcaccttatcctggcatccctgttgaggagctttttaaattgctcaaagagggccacagaatggactgtcctagtaactgcacccatgagctgtatatgctcatgcgcgagtgctggcatgcggtgcctagtcaaaggccaaccttcaaacagctcgtcgaaggcctggacaagattcttgctgcaataagcgaggagtacctcgacttgtctatgccattcgagcaatactcaccttcttgtgaagacacgacgagtacatgcagcagcgacgactctgtatttacacacgaccctttgccccttgctccttgcctgtttgcctgtcctagtggccgcacctag

SEQ ID NO: 105 - DNA, 462 bp, synthetic construct
  source 1..462: mol_type = other DNA
atggaaaaaatcaacagtctttcaacacaattagttaagtgctgcttttgtgatttcttgaaggtgaagatgcacactgtgtcctacattcatttcttctaccttggcctgtgtttgcttaccttaaccagttctgctgctgccggcccagaaacactgtgtggtgctgagctggttgatgctcttcagttcgtatgtggagacagaggcttctacttcagtaagcctacagggtatggatccagcagtagacgcttacaccacaagggaatagtggatgagtgctgcttccagagttgtgacctgaggaggctggagatgtactgtgctccaataaagccacctaaatctgcacgctctgtacgtgctcagcgccacactgatatgccaaaagcacaaaaggaagtgcatttgaagaatacaagtagagggaacacaggaaacagaaactacagaatgtaa

SEQ ID NO: 106 - DNA, 213 bp, synthetic construct
  source 1..213: mol_type = other DNA
ggcccagaaacactgtgtggtgctgagctggttgatgctcttcagttcgtatgtggagacagaggcttctacttcagtaagcctacagggtatggatccagcagtagacgcttacaccacaagggaatagtggatgagtgctgcttccagagttgtgacctgaggaggctggagatgtactgtgctccaataaagccacctaaatctgcataa

SEQ ID NO: 107 - DNA, 4092 bp, synthetic construct
  source 1..4092: mol_type = other DNA
atgaaatctggggctgggggagggaccctcgccgtattctgtgggcttttgttggcgttcgccgcactctgtctctgtccgaccaatggtgaaatatgcggcccgaatgtggatattagaaacgatattcacgagctcaagaggctcgaaaattgtactgtagtggaggggtttctgcaaatactgcttatcagtaaggcggaagattaccgcaacttccgctttcctaagctgactgtgatcacagactatctcctgctgttcagagttgccggacttgaatccctctcagatctgtttcctaacctcacagtgatcaggggtagaaacctgttctacaactatgctctcgtaatatttgagatgacgaaccttaaggagattggactccacaatcttcgcaacataaccaggggtgctataaggatcgaaaaaaatagcgacctgtgctatctttctacagtagactggagtctcatattggacgcagtatccaacaactatatagttggcaacaagcctcccaaagaatgcggtgatctctgtcctggaactatggaggaaaaacctctctgcgaaaagactagtataaacaacgagtacaactataggtgttggactaccaaccactgccagaagatgtgcccgagcagctgcggcaagcgcgcgtgcaccgatcagaatgaatgctgccaccccgaatgtcttggtagctgtacggctcccgacaataacacagcctgcgttgcgtgtcgcaattactattatgagggtgtctgtatgcctacctgtccgcctaacacatataagttcgaggggtggcgctgcgtgacaaaagaattttgttccaaggtcccagcaacggagacgtctgactatgaaaggtttgtaattcataatgatgaatgtatggcggaatgcccatctggatttatcaggaacggtagtcagagcatgttttgctccccatgtgaaggcccatgtcccaaaatttgtgaagacgggaagacgaagacaatagatagcgtcacttctgctcaaatgcttcagggatgtaccatcttgaaaggaaatctcttgattaacattcgccggggtaataatatagcaagtgagcttgaaaactttatggggctcatagaaacggtaactgggtatgtcaagatccgccatagtcatgcactcgtgtcactttcattcttgaagaatctccgctatatactcggcgaagagcaagttgacggcaactactcattttacgttctcgataatcataatttgcagcagctttgggactggaatcaccacaacttgaccattaaagaaggaaagatgtacttcgctttcaatccgaaactttgtgtatccgaaatttaccgcatggaagaggtgtctggaactaaaggacgccagtcaaaaggagatataaatcccaggaataatggagaaagggcgtcctgtgagagccatatattgagattcgtgagcaataccacgctgaagaaccggataaaactcacctgggagagatacaggcccccggattaccgggacctcatctctttcacggtttattacaaggaggcccccttcaaaaacgtcacagagtacgatgggcaagatgcctgcggctccaactcttggaacatggtcgacgtagacttgccccctaacaaagagaatgaccctggtatattgcttcaaggacttaaaccttggacgcagtacgccatatacgtcaaagccgtgacccttacaatgatggaaaaccaccacatccacggggctaaatccgagattgtttatataaggacaaatgcagccgtccccagcatacctctcgacgtaatatctgcctctaatagcagcagccagctcattgtgaagtggaatcctccttcacttcccaacgggaatctgtcttattacattgtccgctggcaacaacaacctcaggattcttatttgtatcggcataactactgtagtaaagacaaggtgccgattcgccggtacgcggacggtactattgacacggaagaagcaacagagccaacaaaacccgaaggatgcggcggtgaaaaaggtccgtgttgtgcgtgccctaagaccgaagctgagaagcaagctgaaaaggaagaagctgaataccgcaaggtgtttgagaacttcctgcataactccatcttcgttcctcgcccggatagaaagaggcgcgacgtgttcaggattgctaacgccacgctcgctacgaggaatcggaacattactggtgcggatcacttcaccaatgcatccgatgcggaagagtccgaagtagagtacccattttttgagacgaaagtcgacgggaaggagagaacggtaatatctcatttgcagccttttaccctttatagaatagatatccacagctgcaatcacgaagcagacacccttggttgtagtgcttctaacttcgtgtttgcccgcacgatgccctctgaaggcgctgacaatatacccggaaccgtagcatgggaagcgaaggaggagaacacggtttatcttaaatggttggaacccaccaaccctaacggacttatcttgatgtacgagattaaatatggacaacacggggaggaaaagagggagtgtgtcagtaggcaagaatacaaaaaacttggtggcgccaaattgacacacttgaaccctggtaattacagcgcgcgcgtgcaggcgactagcttggcaggtaatggttcttggactgaacccgtctccttctatgttcagcccaaatccgcaaattacgacaactttctccatttgataatagtccttccaatagccttcctgttgataataggtggcctcctcattatgttgtacgtctttaataagaaacggaactctgaccggctcggaaatggtgttctgtatgcaagtgttaatccagaatatttctctgcatcagacgtttatgtgccggacgagtgggaagtgccaagggagaaaattacaatgtgcagagaactcggacagggttcttttggcatggtttacgagggcatcgcaaaaggcgtggtgaaggacgaacccgagacgagagttgctatcaaaacagtcaatgagtcagcttctatgcgcgaacggattgagtttttgaatgaggccagtgtaatgaaggagtttaactgccaccacgtcgtacggttgcttggcgtcgtgtctcaaggacagcccaccctcgttattatggagttgatgacccggggggatctcaagtcctacttgaggtctcttcgccctgacactgagtcaaacccagggcaggcacctcctacgctgaaaaaaatgatacaaatggctggagaaatcgccgatgggatggcgtatctcaacgctaataagtttgtacaccgcgatctggccgcaagaaattgtatggtcgcagaggattttactgtcaagatcggcgatttcggcatgacacgcgacatctatgagaccgactactataggaagggggggaagggtttgctcccagtaaggtggatgtcaccggagagcctcaaggatggggtgttcacgactcacagcgatgtctggtcttttggagttgtactctgggaaatagctacactggcagagcaaccctaccaaggaatgacaaatgaacaagtcctccgctttgtaatggaagggggtcttctcgaaaaaccggacaactgcccagacatgcttttcgagctgatgcgcatgtgctggcagtacaatcccaaaatgcgcccgtcttttcttgaaataatctcaagcatcaaggatgaacttgacccggcctttaaagaagtatcatttttctattccgaagaaaataaaccgccagatacagaagaactcgaccttgagacagaaaacatggaaagtattcctctcgatccttcctcaactttgcagccgacggataaacattctggccacaaggcggaaaatggcccgggggttgtagtactcagggcatcatttgaagaaaggcagccatacgcacatatgaatgggggtcgcaagaacgagcgggctttgcctcttccccaatcttctgcatgctag

SEQ ID NO: 108 - DNA, 4098 bp, synthetic construct
  source 1..4098: mol_type = other DNA
gaattcatgaaatctggggctgggggagggaccctcgccgtattctgtgggcttttgttggcgttcgccgcactctgtctctgtccgaccaatggtgaaatatgcggcccgaatgtggatattagaaacgatattcacgagctcaagaggctcgaaaattgtactgtagtggaggggtttctgcaaatactgcttatcagtaaggcggaagattaccgcaacttccgctttcctaagctgactgtgatcacagactatctcctgctgttcagagttgccggacttgaatccctctcagatctgtttcctaacctcacagtgatcaggggtagaaacctgttctacaactatgctctcgtaatatttgagatgacgaaccttaaggagattggactccacaatcttcgcaacataaccaggggtgctataaggatcgaaaaaaatagcgacctgtgctatctttctacagtagactggagtctcatattggacgcagtatccaacaactatatagttggcaacaagcctcccaaagaatgcggtgatctctgtcctggaactatggaggaaaaacctctctgcgaaaagactagtataaacaacgagtacaactataggtgttggactaccaaccactgccagaagatgtgcccgagcagctgcggcaagcgcgcgtgcaccgatcagaatgaatgctgccaccccgaatgtcttggtagctgtacggctcccgacaataacacagcctgcgttgcgtgtcgcaattactattatgagggtgtctgtatgcctacctgtccgcctaacacatataagttcgaggggtggcgctgcgtgacaaaagaattttgttccaaggtcccagcaacggagacgtctgactatgaaaggtttgtaattcataatgatgaatgtatggcggaatgcccatctggatttatcaggaacggtagtcagagcatgttttgctccccatgtgaaggcccatgtcccaaaatttgtgaagacgggaagacgaagacaatagatagcgtcacttctgctcaaatgcttcagggatgtaccatcttgaaaggaaatctcttgattaacattcgccggggtaataatatagcaagtgagcttgaaaactttatggggctcatagaaacggtaactgggtatgtcaagatccgccatagtcatgcactcgtgtcactttcattcttgaagaatctccgctatatactcggcgaagagcaagttgacggcaactactcattttacgttctcgataatcataatttgcagcagctttgggactggaatcaccacaacttgaccattaaagaaggaaagatgtacttcgctttcaatccgaaactttgtgtatccgaaatttaccgcatggaagaggtgtctggaactaaaggacgccagtcaaaaggagatataaatcccaggaataatggagaaagggcgtcctgtgagagccatatattgagattcgtgagcaataccacgctgaagaaccggataaaactcacctgggagagatacaggcccccggattaccgggacctcatctctttcacggtttattacaaggaggcccccttcaaaaacgtcacagagtacgatgggcaagatgcctgcggctccaactcttggaacatggtcgacgtagacttgccccctaacaaagagaatgaccctggtatattgcttcaaggacttaaaccttggacgcagtacgccatatacgtcaaagccgtgacccttacaatgatggaaaaccaccacatccacggggctaaatccgagattgtttatataaggacaaatgcagccgtccccagcatacctctcgacgtaatatctgcctctaatagcagcagccagctcattgtgaagtggaatcctccttcacttcccaacgggaatctgtcttattacattgtccgctggcaacaacaacctcaggattcttatttgtatcggcataactactgtagtaaagacaaggtgccgattcgccggtacgcggacggtactattgacacggaagaagcaacagagccaacaaaacccgaaggatgcggcggtgaaaaaggtccgtgttgtgcgtgccctaagaccgaagctgagaagcaagctgaaaaggaagaagctgaataccgcaaggtgtttgagaacttcctgcataactccatcttcgttcctcgcccggatagaaagaggcgcgacgtgttcaggattgctaacgccacgctcgctacgaggaatcggaacattactggtgcggatcacttcaccaatgcatccgatgcggaagagtccgaagtagagtacccattttttgagacgaaagtcgacgggaaggagagaacggtaatatctcatttgcagccttttaccctttatagaatagatatccacagctgcaatcacgaagcagacacccttggttgtagtgcttctaacttcgtgtttgcccgcacgatgccctctgaaggcgctgacaatatacccggaaccgtagcatgggaagcgaaggaggagaacacggtttatcttaaatggttggaacccaccaaccctaacggacttatcttgatgtacgagattaaatatggacaacacggggaggaaaagagggagtgtgtcagtaggcaagaatacaaaaaacttggtggcgccaaattgacacacttgaaccctggtaattacagcgcgcgcgtgcaggcgactagcttggcaggtaatggttcttggactgaacccgtctccttctatgttcagcccaaatccgcaaattacgacaactttctccatttgataatagtccttccaatagccttcctgttgataataggtggcctcctcattatgttgtacgtctttaataagaaacggaactctgaccggctcggaaatggtgttctgtatgcaagtgttaatccagaatatttctctgcatcagacgtttatgtgccggacgagtgggaagtgccaagggagaaaattacaatgtgcagagaactcggacagggttcttttggcatggtttacgagggcatcgcaaaaggcgtggtgaaggacgaacccgagacgagagttgctatcaaaacagtcaatgagtcagcttctatgcgcgaacggattgagtttttgaatgaggccagtgtaatgaaggagtttaactgccaccacgtcgtacggttgcttggcgtcgtgtctcaaggacagcccaccctcgttattatggagttgatgacccggggggatctcaagtcctacttgaggtctcttcgccctgacactgagtcaaacccagggcaggcacctcctacgctgaaaaaaatgatacaaatggctggagaaatcgccgatgggatggcgtatctcaacgctaataagtttgtacaccgcgatctggccgcaagaaattgtatggtcgcagaggattttactgtcaagatcggcgatttcggcatgacacgcgacatctatgagaccgactactataggaagggggggaagggtttgctcccagtaaggtggatgtcaccggagagcctcaaggatggggtgttcacgactcacagcgatgtctggtcttttggagttgtactctgggaaatagctacactggcagagcaaccctaccaaggaatgacaaatgaacaagtcctccgctttgtaatggaagggggtcttctcgaaaaaccggacaactgcccagacatgcttttcgagctgatgcgcatgtgctggcagtacaatcccaaaatgcgcccgtcttttcttgaaataatctcaagcatcaaggatgaacttgacccggcctttaaagaagtatcatttttctattccgaagaaaataaaccgccagatacagaagaactcgaccttgagacagaaaacatggaaagtattcctctcgatccttcctcaactttgcagccgacggataaacattctggccacaaggcggaaaatggcccgggggttgtagtactcagggcatcatttgaagaaaggcagccatacgcacatatgaatgggggtcgcaagaacgagcatgctttgcctcttccccaatcttctgcatgctag

SEQ ID NO: 109 - DNA, 3264 bp, synthetic construct
  source 1..3264: mol_type = other DNA
atgggtactcccccaaggacgttcctgatcctgggatgttttctcacaggaccgctcctaacactttgccagcttcctctgccgactattgttcccaatagaaatgagatggttgtacagctgaattccaatttcacactcaaatgctctggagacagcgaagtgagctggcagtacccagtgaccgagggaagccacaggatagacatcagacacgaggagaacaacagtggcctcttcgtgacagtgcttgaagtcggaaatgcctcagccgctcacacgggcatgtatgtttgctattataaccacacgcaagtggaggatggggaagtcgaggggaaggacatctacatctatgtgcctgacccagacatgcctttcgttccttccttaccagaagaccagttcatcctagtagaagaaggtgatcccactgttatcccttgtcggacaagtgacccaagtgctgaagtgactttagttaacagtttagacaagcctgtctatgctttctatgacagcaaacagggcttcgtagggaacttccttgcaggaccatacacatgcaaaacaatggttaaaggcgtggagttcaagtccgatgagttcctcatctatattttaagagctacttcacagctgccggttgaaattgaagctctgaaaactgtctacaaaacaggcgagaccatcgtagtaacttgtgtggtctttgacaatgaggtggttaatttacagtggaattatcccgggaaagtgaaagaaaaaggtctgataaaacttgatgatatcaaagtcccatcacagaagttggtttacacgttgaccatacctgacgcatcagtgaaagacacaggggattatgaatgtactgcccgacatgcaaccaaggaggttaaggaaaataagaaagtagtcattacagttcatgacaaagggttcattcatctagagcctcaatttagccctttggaagctgtcaatctacatgaagtcaaaaattttgtcgtcgatgtgcaggcgtaccccgctccaaaaatgtactggttgaaggataatgtgactctgattgaaaaccttactgagattgttactagttcaaacagagtccaggaaacacggtttcaaagtgtactaaaattgatccgggccaaggaagaagacagtgggtactatactttggttgctgaaaatgaagatgagattaagagatacaccttctcgttgctaatacaagttccagccttgatcttagacctcatggacgaccaccaaggctctgctggcaggcagacggtgaggtgcttggctgaaggtaccccgcttcctgatgtggaatggttggtttgcaaggacattaaaaaatgcagcaatgacacttcctggactcttctgactaacaatatctctgatatacacatggaagcccacctggatgagaggaatatggtggaaagccaggtgaccttccagaaggtagaagaaaccctggctgtgagatgtgtagcaagaaacgaccttggagctgttactcgggaactgaaacttgtggctcccaccttgcgatcagaactgacggtggctgctgctgtcttagtactgctggtgattgtgataatttcactgattgtcctggtcatcatctggaaacagaagccaagatatgagataagatggagagtcattgagtctatcagccccgatggccatgaatacatttatgtggacccaatgcagctaccttatgactccagatgggagtttcctcgagatgggttagtgcttggtcgaatccttggttctggtgcatttggaaaagtggtggaagggacagcatatggattgagtcgttctcaacctgtgatgaaagtagccgtgaaaatgctgaaacctacagctagatccagtgaaaaacaggcgctcatgtctgaactgaagataatgacacatcttgggccccacctgaatattgtgaacctgcttggagcttgtacgaaatcaggtcctatttacataatcactgaatactgcttttacggtgatttggtgaactatctgcacaagaacagggacaacttcctcagccgacatccagagaaaccaaagaaagatctggatatttttgggatgaacccagctgatgaaagcacaagaagctatgtgattttatcatttgaaaacaccggagaatatatggatatgaaacaagctgataccactcagtatgtgccaatgctggaaaggaaggagggatctaaatactctgatattcagagatctgtatatgatcgacctgcttcatataagaagaaatctttgtcagaatcagaagtaaaaaaccttctttcagatgacggttcggagggtctaagcctactggatttgctaagcttcacctaccaggttgcacggggaatggaattcttggcttctaaaaattgcgtacaccgtgacttggcagctcgtaatgtccttctggctcaaggcaaaatcgtgaagatctgcgactttgggttggctagagacatcatgcatgattccaactatgtctccaagggcagcaccttcctcccagtaaaatggatggcacctgaaagcatttttgacaatctgtacacaacattaagtgatgtctggtcttatggcattctgctgtgggaaatattttctcttggtggcacaccatatcctggcatgatggtcgactccaccttctacaataagataaagagtggctaccgaatggcaaaacctgatcatgctaccaatgaagtgtatgagatcatggtaaagtgctggaacagtgaaccagagaaaagaccttcgttttaccatctgagtgaaattgtggagagcttgttgcctggagagtacaaaaagagctacgagaagattcacctggacttcctgaaaagcgatcacccagctgtcactcgaatgagaggggactgtgacaatgcttacattggtgtcacctacaagaatgaagacaagataaaggatagagagagtggatttgatgagcagaggctgagtgctgacagtgggtacatcatccccctgcctgacattgaccctgtttctgaagatgagcttggcaaaaggaacaggcacagttcccagacatctgaagagagtgccattgaaaccggttccagtagctctacctttataaagagagaggatgagaccattgaggacattgacatgatggatgacattggaattgactcctcggatcttgtagaggacagcttcctgtaa

SEQ ID NO: 110 - DNA, 3264 bp, synthetic construct
  source 1..3264: mol_type = other DNA
atgggtactcccccaaggacgttcctgatcctgggatgttttctcacaggaccgctcctaacactttgccagcttcctctgccgactattgttcccaatagaaatgagatggttgtacagctgaattccaatttcacactcaaatgctctggagacagcgaagtgagctggcagtacccagtgaccgagggaagccacaggatagacatcagacacgaggagaacaacagtggcctcttcgtgacagtgcttgaagtcggaaatgcctcagccgctcacacgggcatgtatgtttgctattataaccacacgcaagtggaggatggggaagtcgaggggaaggacatctacatctatgtgcctgacccagacatgcctttcgttccttccttaccagaagaccagttcatcctagtagaagaaggtgatcccactgttatcccttgtcggacaagtgacccaagtgctgaagtgactttagttaacagtttagacaagcctgtctatgctttctatgacagcaaacagggcttcgtagggaacttccttgcaggaccatacacatgcaaaacaatggttaaaggcgtggagttcaagtccgatgagttcctcatctatattttaagagctacttcacagctgccggttgaaattgaagctctgaaaactgtctacaaaacaggcgagaccatcgtagtaacttgtgtggtctttgacaatgaggtggttaatttacagtggaattatcccgggaaagtgaaagaaaaaggtctgataaaacttgatgatatcaaagtcccatcacagaagttggtttacacgttgaccatacctgacgcatcagtgaaagacacaggggattatgaatgtactgcccgacatgcaaccaaggaggttaaggaaaataagaaagtagtcattacagttcatgacaaagggttcattcatctagagcctcaatttagccctttggaagctgtcaatctacatgaagtcaaaaattttgtcgtcgatgtgcaggcgtaccccgctccaaaaatgtactggttgaaggataatgtgactctgattgaaaaccttactgagattgttactagttcaaacagagtccaggaaacacggtttcaaagtgtactaaaattgatccgggccaaggaagaagacagtgggtactatactttggttgctgaaaatgaagatgagattaagagatacaccttctcgttgctaatacaagttccagccttgatcttagacctcatggacgaccaccaaggctctgctggcaggcagacggtgaggtgcttggctgaaggtaccccgcttcctgatgtggaatggttggtttgcaaggacattaaaaaatgcagcaatgacacttcctggactcttctgactaacaatatctctgatatacacatggaagcccacctggatgagaggaatatggtggaaagccaggtgaccttccagaaggtagaagaaaccctggctgtgagatgtgtagcaagaaacgaccttggagctgttactcgggaactgaaacttgtggctcccaccttgcgatcagaactgacggtggctgctgctgtcttagtactgctggtgattgtgataatttcactgattgtcctggtcatcatctggaaacagaagccaagatatgagataagatggagagtcattgagtctatcagccccgatggccatgaatacatttatgtggacccaatgcagctaccttatgactccagatgggagtttcctcgagatgggttagtgcttggtcgaatccttggttctggtgcatttggaaaagtggtggaagggacagcatatggattgagtcgttctcaacctgtgatgaaagtagccgtgaaaatgctgaaacctacagctagatccagtgaaaaacaggcgctcatgtctgaactgaagataatgacacatcttgggccccacctgaatattgtgaacctgcttggagcttgtacgaaatcaggtcctatttacataatcactgaatactgcttttacggtgatttggtgaactatctgcacaagaacagggacaacttcctcagccgacatccagagaaaccaaagaaagatctggatatttttgggatgaacccagctgatgaaagcacaagaagctatgtgattttatcatttgaaaacaccggagaatatatggatatgaaacaagctgataccactcagtatgtgccaatgctggaaaggaaggagggatctaaatactctgatattcagagatctgtatatgatcgacctgcttcatataagaagaaatctttgtcagaatcagaagtaaaaaaccttctttcagatgacggttcggagggtctaagcctactggatttgctaagcttcacctaccaggttgcacggggaatggaattcttggcttctaaaaattgcgtacaccgtgacttggcagctcgtaatgtccttctggctcaaggcaaaatcgtgaagatctgcgactttgggttggctagagtcatcatgcatgattccaactatgtctccaagggcagcaccttcctcccagtaaaatggatggcacctgaaagcatttttgacaatctgtacacaacattaagtgatgtctggtcttatggcattctgctgtgggaaatattttctcttggtggcacaccatatcctggcatgatggtcgactccaccttctacaataagataaagagtggctaccgaatggcaaaacctgatcatgctaccaatgaagtgtatgagatcatggtaaagtgctggaacagtgaaccagagaaaagaccttcgttttaccatctgagtgaaattgtggagagcttgttgcctggagagtacaaaaagagctacgagaagattcacctggacttcctgaaaagcgatcacccagctgtcactcgaatgagaggggactgtgacaatgcttacattggtgtcacctacaagaatgaagacaagataaaggatagagagagtggatttgatgagcagaggctgagtgctgacagtgggtacatcatccccctgcctgacattgaccctgtttctgaagatgagcttggcaaaaggaacaggcacagttcccagacatctgaagagagtgccattgaaaccggttccagtagctctacctttataaagagagaggatgagaccattgaggacattgacatgatggatgacattggaattgactcctcggatcttgtagaggacagcttcctgtaa

SEQ ID NO: 111 - DNA, 3273 bp, synthetic construct
  source 1..3273: mol_type = other DNA
atgctctgtccctctctgaaggcatctctgcagctcctcatcctcactggtctgctggaggtaacgtctggaggcagcgggctgcacatcgaacctgaagatgctgagctcgtccttaggctccacagcactttctccctcgtgtgctatggggacggcacgctggtctgggagcgggatggtcagcctctcactgccgtgctggagcacagggacggggtcttcatcagcaacctcaccctcaggaacgtgacaggccgtcacacgggggagtatgcgtgcttctacagccctgaccaggctccggagcgagcagagaggaaagccctttacatctatgttccagatccctccttagtttttctccccgcaatcacttctgaagagttcttcatcttcatcacgggctacacagaggccaccatcccatgccgtgtgaccaacccagagctgcaggtgaccctctatgaaaagaaagtggagaatcccattccagctacttatgacccacaacagggcttcaaaggcttctttgaggacaagacctactactgccaggcaatcgtggatgaccaagaggtggattcagacaccttctatgtctaccggatccaggtctcatctgtgaacgtctccatcagcgcagtgcagaccgtagtgcggcagggagaaaatgttaccctgatgtgcactgtcagtggcaatgagctggtcaatttcaactgggattatccccgcaagcaggcagggaaggctgtggagccagtgaccgatttcctgcctggatccacccatgacatccgttccatcctcatcatccagaatgcagagctagaggacagtgggacctacgtctgcaatgtctctgagggctaccatgagaagacagaccggaaagacatcacggtccaagtgatcgagcgtggctttgtacgcttccacacccacctggccagcacggtgtatgctgaggtccacaagagccacatcatccaggtggatgtggaggcctacccacagccaaacattgtgtggctgaagaacaacaagacattgaccatggagagcagcagcgagttcaccatcaccaacaggaacctgtcagaaaccaggtatcagacgtctctggtcctggtgcgtgtgaagcaggaggaaggaggatattacaccatccgagcttccaatgaggatgatgcacaagagctgtccttccatctgcagataaatgtgccagccaaagtggtggatctcaaggaaaacagcagtgccagcagcggggagcagactgtaacgtgctctgctgaagggatgccccagccagagatcagttggtccacttgcagcaacatcaaatggtgtggcagccaggggcaacccacccagctgctggggaacaactctgcagagattggcctgcacactaatgctacgtaccatgcagagctgcaggtgtaccgtgtgaacagcaccctgcagctgcacagggtggatgaacccctgcttctgagatgcaccgtgcaaaacttcctgggctccaactcccaagacatcactctggtcccaaatgccttgccattcaaagtggtcatcatctccgtcatcctggctctgctggtcctcaccgtcatctccctgatcatcctgatcatcctgtggcagaagaaacctcgctatgagatccgctggaaggtgatcgagtcagtgagctccgatgggcacgagtacatctacgtggatcccatgcagctcccttatgactccagctgggaggtgcccagggacaagctggtgttaggacgcactcttggctccggtgcctttggacgcgtggtggaggcaacagcgcatggcctgagccattcacagtccaccatgaaagtggcagtcaagatgctcaagtccactgcacggagcagtgagaagcaagccctcatgtctgagctgaagatcatgagccacctgggacctcacctcaacatcgtcaacttgctgggggcctgcaccaaaggagggcccatctatatcatcaccgagtactgccgttatggggacctggtggactacctgcaccgcaacaagcacaccttcctgcagtcctatggcgagaaggcccgccgggaggcagagctgtatgggaataccatcaaggaggaccacgtgcagagtcacctctccttgtctgtcgagagtgatgggggctacatggacatgagcaaggatgagtctctggattacgtgcccatgtctgacatgaagggtgaagtcaagtatgctgacatcgagtcttctaactatggcaccccatatgagctggacagctattccccatcagctccggaaagaacagaccgggtgacactgataaatgaatctccactcctcagctacatggacttggtgggcttcagcttccaggtggccaatgggatggagttcctggcttccaaaaattgtgtgcatcgtgacctggctgccaggaacgtcctcatctgcgaggggaagctggtgaagatctgtgactttggtctggcaagagacatcatgagggattccaactacatctccaaaggcagtaccttcttgccccttaagtggatggccccagagagcatcttcaacaacctctacaccaccctaagtgatgtgtggtcctttgggattcttctctgggagatattcactctaggagggactccctaccctgaactgcctatgaacgaacagttctacaatgccatcaaacgtggctatcggatgtccaaacctacccatgcttctgatgaaatctacgatatcatgcagaagtgctgggaggagaagtttgagatcagaccgtccttctcacagctggtggtgcttatgggaaacctcttggtggactgctacagaaagaggtaccaacaggtagatgaagagttcatgaagagcgaccaccccgctgttgttcgcacaagacccaccatccccgggctgaacaacgccaggctccctcccagctcccccaccctctacacggctgtgcaccagaacgggggagagaacgactacatcatccctcttcctgaccccaagcctgatgcaatctgtgacctccctcaggaggcctccgtcagccgtgccagctctatgctgaatgaagccaacacatcatctacaatatcctgtgacagccccctgggcccccggcaggacgaggagccagaatgtgacctgcagctgggctgccaggagctggccccgggtcaccacgaggtggaggagagctttctgtag

SEQ ID NO: 112 - DNA, 3273 bp, synthetic construct
  source 1..3273: mol_type = other DNA
atgctctgtccctctctgaaggcatctctgcagctcctcatcctcactggtctgctggaggtaacgtctggaggcagcgggctgcacatcgaacctgaagatgctgagctcgtccttaggctccacagcactttctccctcgtgtgctatggggacggcacgctggtctgggagcgggatggtcagcctctcactgccgtgctggagcacagggacggggtcttcatcagcaacctcaccctcaggaacgtgacaggccgtcacacgggggagtatgcgtgcttctacagccctgaccaggctccggagcgagcagagaggaaagccctttacatctatgttccagatccctccttagtttttctccccgcaatcacttctgaagagttcttcatcttcatcacgggctacacagaggccaccatcccatgccgtgtgaccaacccagagctgcaggtgaccctctatgaaaagaaagtggagaatcccattccagctacttatgacccacaacagggcttcaaaggcttctttgaggacaagacctactactgccaggcaatcgtggatgaccaagaggtggattcagacaccttctatgtctaccggatccaggtctcatctgtgaacgtctccatcagcgcagtgcagaccgtagtgcggcagggagaaaatgttaccctgatgtgcactgtcagtggcaatgagctggtcaatttcaactgggattatccccgcaagcaggcagggaaggctgtggagccagtgaccgatttcctgcctggatccacccatgacatccgttccatcctcatcatccagaatgcagagctagaggacagtgggacctacgtctgcaatgtctctgagggctaccatgagaagacagaccggaaagacatcacggtccaagtgatcgagcgtggctttgtacgcttccacacccacctggccagcacggtgtatgctgaggtccacaagagccacatcatccaggtggatgtggaggcctacccacagccaaacattgtgtggctgaagaacaacaagacattgaccatggagagcagcagcgagttcaccatcaccaacaggaacctgtcagaaaccaggtatcagacgtctctggtcctggtgcgtgtgaagcaggaggaaggaggatattacaccatccgagcttccaatgaggatgatgcacaagagctgtccttccatctgcagataaatgtgccagccaaagtggtggatctcaaggaaaacagcagtgccagcagcggggagcagactgtaacgtgctctgctgaagggatgccccagccagagatcagttggtccacttgcagcaacatcaaatggtgtggcagccaggggcaacccacccagctgctggggaacaactctgcagagattggcctgcacactaatgctacgtaccatgcagagctgcaggtgtaccgtgtgaacagcaccctgcagctgcacagggtggatgaacccctgcttctgagatgcaccgtgcaaaacttcctgggctccaactcccaagacatcactctggtcccaaatgccttgccattcaaagtggtcatcatctccgtcatcctggctctgctggtcctcaccgtcatctccctgatcatcctgatcatcctgtggcagaagaaacctcgctatgagatccgctggaaggtgatcgagtcagtgagctccgatgggcacgagtacatctacgtggatcccatgcagctcccttatgactccagctgggaggtgcccagggacaagctggtgttaggacgcactcttggctccggtgcctttggacgcgtggtggaggcaacagcgcatggcctgagccattcacagtccaccatgaaagtggcagtcaagatgctcaagtccactgcacggagcagtgagaagcaagccctcatgtctgagctgaagatcatgagccacctgggacctcacctcaacatcgtcaacttgctgggggcctgcaccaaaggagggcccatctatatcatcaccgagtactgccgttatggggacctggtggactacctgcaccgcaacaagcacaccttcctgcagtcctatggcgagaaggcccgccgggaggcagagctgtatgggaataccatcaaggaggaccacgtgcagagtcacctctccttgtctgtcgagagtgatgggggctacatggacatgagcaaggatgagtctctggattacgtgcccatgtctgacatgaagggtgaagtcaagtatgctgacatcgagtcttctaactatggcaccccatatgagctggacagctattccccatcagctccggaaagaacagaccgggtgacactgataaatgaatctccactcctcagctacatggacttggtgggcttcagcttccaggtggccaatgggatggagttcctggcttccaaaaattgtgtgcatcgtgacctggctgccaggaacgtcctcatctgcgaggggaagctggtgaagatctgtgactttggtctggcaagagacatcatgaggaattccaactacatctccaaaggcagtaccttcttgccccttaagtggatggccccagagagcatcttcaacaacctctacaccaccctaagtgatgtgtggtcctttgggattcttctctgggagatattcactctaggagggactccctaccctgaactgcctatgaacgaacagttctacaatgccatcaaacgtggctatcggatgtccaaacctacccatgcttctgatgaaatctacgatatcatgcagaagtgctgggaggagaagtttgagatcagaccgtccttctcacagctggtggtgcttatgggaaacctcttggtggactgctacagaaagaggtaccaacaggtagatgaagagttcatgaagagcgaccaccccgctgttgttcgcacaagacccaccatccccgggctgaacaacgccaggctccctcccagctcccccaccctctacacggctgtgcaccagaacgggggagagaacgactacatcatccctcttcctgaccccaagcctgatgcaatctgtgacctccctcaggaggcctccgtcagccgtgccagctctatgctgaatgaagccaacacatcatctacaatatcctgtgacagccccctgggcccccggcaggacgaggagccagaatgtgacctgcagctgggctgccaggagctggccccgggtcaccacgaggtggaggagagctttctgtag

SEQ ID NO: 113 - DNA, 3273 bp, synthetic construct
  source 1..3273: mol_type = other DNA
atgctctgtccctctctgaaggcatctctgcagctcctcatcctcactggtctgctggaggtaacgtctggaggcagcgggctgcacatcgaacctgaagatgctgagctcgtccttaggctccacagcactttctccctcgtgtgctatggggacggcacgctggtctgggagcgggatggtcagcctctcactgccgtgctggagcacagggacggggtcttcatcagcaacctcaccctcaggaacgtgacaggccgtcacacgggggagtatgcgtgcttctacagccctgaccaggctccggagcgagcagagaggaaagccctttacatctatgttccagatccctccttagtttttctccccgcaatcacttctgaagagttcttcatcttcatcacgggctacacagaggccaccatcccatgccgtgtgaccaacccagagctgcaggtgaccctctatgaaaagaaagtggagaatcccattccagctacttatgacccacaacagggcttcaaaggcttctttgaggacaagacctactactgccaggcaatcgtggatgaccaagaggtggattcagacaccttctatgtctaccggatccaggtctcatctgtgaacgtctccatcagcgcagtgcagaccgtagtgcggcagggagaaaatgttaccctgatgtgcactgtcagtggcaatgagctggtcaatttcaactgggattatccccgcaagcaggcagggaaggctgtggagccagtgaccgatttcctgcctggatccacccatgacatccgttccatcctcatcatccagaatgcagagctagaggacagtgggacctacgtctgcaatgtctctgagggctaccatgagaagacagaccggaaagacatcacggtccaagtgatcgagcgtggctttgtacgcttccacacccacctggccagcacggtgtatgctgaggtccacaagagccacatcatccaggtggatgtggaggcctacccacagccaaacattgtgtggctgaagaacaacaagacattgaccatggagagcagcagcgagttcaccatcaccaacaggaacctgtcagaaaccaggtatcagacgtctctggtcctggtgcgtgtgaagcaggaggaaggaggatattacaccatccgagcttccaatgaggatgatgcacaagagctgtccttccatctgcagataaatgtgccagccaaagtggtggatctcaaggaaaacagcagtgccagcagcggggagcagactgtaacgtgctctgctgaagggatgccccagccagagatcagttggtccacttgcagcaacatcaaatggtgtggcagccaggggcaacccacccagctgctggggaacaactctgcagagattggcctgcacactaatgctacgtaccatgcagagctgcaggtgtaccgtgtgaacagcaccctgcagctgcacagggtggatgaacccctgcttctgagatgcaccgtgcaaaacttcctgggctccaactcccaagacatcactctggtcccaaatgccttgccattcaaagtggtcatcatctccgtcatcctggctctgctggtcctcaccgtcatctccctgatcatcctgatcatcctgtggcagaagaaacctcgctatgagatccgctggaaggccatcgagtcagtgagctccgatgggcacgagtacatctacgtggatcccatgcagctcccttatgactccagctgggaggtgcccagggacaagctggtgttaggacgcactcttggctccggtgcctttggacgcgtggtggaggcaacagcgcatggcctgagccattcacagtccaccatgaaagtggcagtcaagatgctcaagtccactgcacggagcagtgagaagcaagccctcatgtctgagctgaagatcatgagccacctgggacctcacctcaacatcgtcaacttgctgggggcctgcaccaaaggagggcccatctatatcatcaccgagtactgccgttatggggacctggtggactacctgcaccgcaacaagcacaccttcctgcagtcctatggcgagaaggcccgccgggaggcagagctgtatgggaataccatcaaggaggaccacgtgcagagtcacctctccttgtctgtcgagagtgatgggggctacatggacatgagcaaggatgagtctctggattacgtgcccatgtctgacatgaagggtgaagtcaagtatgctgacatcgagtcttctaactatggcaccccatatgagctggacagctattccccatcagctccggaaagaacagaccgggtgacactgataaatgaatctccactcctcagctacatggacttggtgggcttcagcttccaggtggccaatgggatggagttcctggcttccaaaaattgtgtgcatcgtgacctggctgccaggaacgtcctcatctgcgaggggaagctggtgaagatctgtgactttggtctggcaagagacatcatgagggattccaactacatctccaaaggcagtaccttcttgccccttaagtggatggccccagagagcatcttcaacaacctctacaccaccctaagtgatgtgtggtcctttgggattcttctctgggagatattcactctaggagggactccctaccctgaactgcctatgaacgaacagttctacaatgccatcaaacgtggctatcggatgtccaaacctacccatgcttctgatgaaatctacgatatcatgcagaagtgctgggaggagaagtttgagatcagaccgtccttctcacagctggtggtgcttatgggaaacctcttggtggactgctacagaaagaggtaccaacaggtagatgaagagttcatgaagagcgaccaccccgctgttgttcgcacaagacccaccatccccgggctgaacaacgccaggctccctcccagctcccccaccctctacacggctgtgcaccagaacgggggagagaacgactacatcatccctcttcctgaccccaagcctgatgcaatctgtgacctccctcaggaggcctccgtcagccgtgccagctctatgctgaatgaagccaacacatcatctacaatatcctgtgacagccccctgggcccccggcaggacgaggagccagaatgtgacctgcagctgggctgccaggagctggccccgggtcaccacgaggtggaggagagctttctgtag

SEQ ID NO: 114 - DNA, 3303 bp, synthetic construct
  source 1..3303: mol_type = other DNA
atggcgaaaggtggcataatcgtcgcgatccttctccttatcgttatgcttgctatagagatattgcttttgataactcttatcattgctgtaacgtctggaggcagcgggctgcacatcgaacctgaagatgctgagctcgtccttaggctccacagcactttctccctcgtgtgctatggggacggcacgctggtctgggagcgggatggtcagcctctcactgccgtgctggagcacagggacggggtcttcatcagcaacctcaccctcaggaacgtgacaggccgtcacacgggggagtatgcgtgcttctacagccctgaccaggctccggagcgagcagagaggaaagccctttacatctatgttccagatccctccttagtttttctccccgcaatcacttctgaagagttcttcatcttcatcacgggctacacagaggccaccatcccatgccgtgtgaccaacccagagctgcaggtgaccctctatgaaaagaaagtggagaatcccattccagctacttatgacccacaacagggcttcaaaggcttctttgaggacaagacctactactgccaggcaatcgtggatgaccaagaggtggattcagacaccttctatgtctaccggatccaggtctcatctgtgaacgtctccatcagcgcagtgcagaccgtagtgcggcagggagaaaatgttaccctgatgtgcactgtcagtggcaatgagctggtcaatttcaactgggattatccccgcaagcaggcagggaaggctgtggagccagtgaccgatttcctgcctggatccacccatgacatccgttccatcctcatcatccagaatgcagagctagaggacagtgggacctacgtctgcaatgtctctgagggctaccatgagaagacagaccggaaagacatcacggtccaagtgatcgagcgtggctttgtacgcttccacacccacctggccagcacggtgtatgctgaggtccacaagagccacatcatccaggtggatgtggaggcctacccacagccaaacattgtgtggctgaagaacaacaagacattgaccatggagagcagcagcgagttcaccatcaccaacaggaacctgtcagaaaccaggtatcagacgtctctggtcctggtgcgtgtgaagcaggaggaaggaggatattacaccatccgagcttccaatgaggatgatgcacaagagctgtccttccatctgcagataaatgtgccagccaaagtggtggatctcaaggaaaacagcagtgccagcagcggggagcagactgtaacgtgctctgctgaagggatgccccagccagagatcagttggtccacttgcagcaacatcaaatggtgtggcagccaggggcaacccacccagctgctggggaacaactctgcagagattggcctgcacactaatgctacgtaccatgcagagctgcaggtgtaccgtgtgaacagcaccctgcagctgcacagggtggatgaacccctgcttctgagatgcaccgtgcaaaacttcctgggctccaactcccaagacatcactctggtcccaaatgccttgccattcaaagtggtcatcatctccgtcatcctggctctgctggtcctcaccgtcatctccctgatcatcctgatcatcctgtggcagaagaaacctcgctatgagatccgctggaaggtgatcgagtcagtgagctccgatgggcacgagtacatctacgtggatcccatgcagctcccttatgactccagctgggaggtgcccagggacaagctggtgttaggacgcactcttggctccggtgcctttggacgcgtggtggaggcaacagcgcatggcctgagccattcacagtccaccatgaaagtggcagtcaagatgctcaagtccactgcacggagcagtgagaagcaagccctcatgtctgagctgaagatcatgagccacctgggacctcacctcaacatcgtcaacttgctgggggcctgcaccaaaggagggcccatctatatcatcaccgagtactgccgttatggggacctggtggactacctgcaccgcaacaagcacaccttcctgcagtcctatggcgagaaggcccgccgggaggcagagctgtatgggaataccatcaaggaggaccacgtgcagagtcacctctccttgtctgtcgagagtgatgggggctacatggacatgagcaaggatgagtctctggattacgtgcccatgtctgacatgaagggtgaagtcaagtatgctgacatcgagtcttctaactatggcaccccatatgagctggacagctattccccatcagctccggaaagaacagaccgggtgacactgataaatgaatctccactcctcagctacatggacttggtgggcttcagcttccaggtggccaatgggatggagttcctggcttccaaaaattgtgtgcatcgtgacctggctgccaggaacgtcctcatctgcgaggggaagctggtgaagatctgtgactttggtctggcaagagacatcatgagggattccaactacatctccaaaggcagtaccttcttgccccttaagtggatggccccagagagcatcttcaacaacctctacaccaccctaagtgatgtgtggtcctttgggattcttctctgggagatattcactctaggagggactccctaccctgaactgcctatgaacgaacagttctacaatgccatcaaacgtggctatcggatgtccaaacctacccatgcttctgatgaaatctacgatatcatgcagaagtgctgggaggagaagtttgagatcagaccgtccttctcacagctggtggtgcttatgggaaacctcttggtggactgctacagaaagaggtaccaacaggtagatgaagagttcatgaagagcgaccaccccgctgttgttcgcacaagacccaccatccccgggctgaacaacgccaggctccctcccagctcccccaccctctacacggctgtgcaccagaacgggggagagaacgactacatcatccctcttcctgaccccaagcctgatgcaatctgtgacctccctcaggaggcctccgtcagccgtgccagctctatgctgaatgaagccaacacatcatctacaatatcctgtgacagccccctgggcccccggcaggacgaggagccagaatgtgacctgcagctgggctgccaggagctggccccgggtcaccacgaggtggaggagagctttctgtag

SEQ ID NO: 115 - DNA, 3327 bp, synthetic construct
  source 1..3327: mol_type = other DNA
atggcggattacaaggatgatgatgacaaaaaaggtggcataatcgtcgcgatccttctccttatcgttatgcttgctatagagatattgcttttgataactcttatcattgctgtaacgtctggaggcagcgggctgcacatcgaacctgaagatgctgagctcgtccttaggctccacagcactttctccctcgtgtgctatggggacggcacgctggtctgggagcgggatggtcagcctctcactgccgtgctggagcacagggacggggtcttcatcagcaacctcaccctcaggaacgtgacaggccgtcacacgggggagtatgcgtgcttctacagccctgaccaggctccggagcgagcagagaggaaagccctttacatctatgttccagatccctccttagtttttctccccgcaatcacttctgaagagttcttcatcttcatcacgggctacacagaggccaccatcccatgccgtgtgaccaacccagagctgcaggtgaccctctatgaaaagaaagtggagaatcccattccagctacttatgacccacaacagggcttcaaaggcttctttgaggacaagacctactactgccaggcaatcgtggatgaccaagaggtggattcagacaccttctatgtctaccggatccaggtctcatctgtgaacgtctccatcagcgcagtgcagaccgtagtgcggcagggagaaaatgttaccctgatgtgcactgtcagtggcaatgagctggtcaatttcaactgggattatccccgcaagcaggcagggaaggctgtggagccagtgaccgatttcctgcctggatccacccatgacatccgttccatcctcatcatccagaatgcagagctagaggacagtgggacctacgtctgcaatgtctctgagggctaccatgagaagacagaccggaaagacatcacggtccaagtgatcgagcgtggctttgtacgcttccacacccacctggccagcacggtgtatgctgaggtccacaagagccacatcatccaggtggatgtggaggcctacccacagccaaacattgtgtggctgaagaacaacaagacattgaccatggagagcagcagcgagttcaccatcaccaacaggaacctgtcagaaaccaggtatcagacgtctctggtcctggtgcgtgtgaagcaggaggaaggaggatattacaccatccgagcttccaatgaggatgatgcacaagagctgtccttccatctgcagataaatgtgccagccaaagtggtggatctcaaggaaaacagcagtgccagcagcggggagcagactgtaacgtgctctgctgaagggatgccccagccagagatcagttggtccacttgcagcaacatcaaatggtgtggcagccaggggcaacccacccagctgctggggaacaactctgcagagattggcctgcacactaatgctacgtaccatgcagagctgcaggtgtaccgtgtgaacagcaccctgcagctgcacagggtggatgaacccctgcttctgagatgcaccgtgcaaaacttcctgggctccaactcccaagacatcactctggtcccaaatgccttgccattcaaagtggtcatcatctccgtcatcctggctctgctggtcctcaccgtcatctccctgatcatcctgatcatcctgtggcagaagaaacctcgctatgagatccgctggaaggtgatcgagtcagtgagctccgatgggcacgagtacatctacgtggatcccatgcagctcccttatgactccagctgggaggtgcccagggacaagctggtgttaggacgcactcttggctccggtgcctttggacgcgtggtggaggcaacagcgcatggcctgagccattcacagtccaccatgaaagtggcagtcaagatgctcaagtccactgcacggagcagtgagaagcaagccctcatgtctgagctgaagatcatgagccacctgggacctcacctcaacatcgtcaacttgctgggggcctgcaccaaaggagggcccatctatatcatcaccgagtactgccgttatggggacctggtggactacctgcaccgcaacaagcacaccttcctgcagtcctatggcgagaaggcccgccgggaggcagagctgtatgggaataccatcaaggaggaccacgtgcagagtcacctctccttgtctgtcgagagtgatgggggctacatggacatgagcaaggatgagtctctggattacgtgcccatgtctgacatgaagggtgaagtcaagtatgctgacatcgagtcttctaactatggcaccccatatgagctggacagctattccccatcagctccggaaagaacagaccgggtgacactgataaatgaatctccactcctcagctacatggacttggtgggcttcagcttccaggtggccaatgggatggagttcctggcttccaaaaattgtgtgcatcgtgacctggctgccaggaacgtcctcatctgcgaggggaagctggtgaagatctgtgactttggtctggcaagagacatcatgagggattccaactacatctccaaaggcagtaccttcttgccccttaagtggatggccccagagagcatcttcaacaacctctacaccaccctaagtgatgtgtggtcctttgggattcttctctgggagatattcactctaggagggactccctaccctgaactgcctatgaacgaacagttctacaatgccatcaaacgtggctatcggatgtccaaacctacccatgcttctgatgaaatctacgatatcatgcagaagtgctgggaggagaagtttgagatcagaccgtccttctcacagctggtggtgcttatgggaaacctcttggtggactgctacagaaagaggtaccaacaggtagatgaagagttcatgaagagcgaccaccccgctgttgttcgcacaagacccaccatccccgggctgaacaacgccaggctccctcccagctcccccaccctctacacggctgtgcaccagaacgggggagagaacgactacatcatccctcttcctgaccccaagcctgatgcaatctgtgacctccctcaggaggcctccgtcagccgtgccagctctatgctgaatgaagccaacacatcatctacaatatcctgtgacagccccctgggcccccggcaggacgaggagccagaatgtgacctgcagctgggctgccaggagctggccccgggtcaccacgaggtggaggagagctttctgtag

SEQ ID NO: 116 - DNA, 756 bp, synthetic construct
  source 1..756: mol_type = other DNA
atgtgcccgcagccggcaaggcttgaacccggcatgaatttcggcgtggtcttcgccgtcatcctctccctgcccctggcccgcctggagggggaccccatacccgaagatatttatgagattttgggtggcagctccgtgcgctccatcagtgacctccagcgtgccctgcggatagactccgtagaggaggacagctctagcctggacctgaatgcaactcagcccagccaaaaccatgtgtccctgtctcgagagaggcgaagccttgatgctctggcagcagcagagccagctgtcctcgccgagtgcaagacacggacggtggtctttgagatctcccgtgacatggtggacagcaccaatgccaacttcgtggtgtggccaccctgcgtggaggtgcagcggtgctccggctgctgcaacaaccgcaacgtgcagtgccgccccatgcagattcgcgtccggcatgtccaggtgaacaagattgagtttttccagaggaagccaatattcaaaaaagtcatcgtgcctttggaggaccacgtgcagtgccggtgcgaagtggtgtcccggccgccacccaggagcaaccgaccggcatcccgtgagcagagacgcttctcgccgtcattcaccacagccgccatctcccagaggaagcgggtacgccggccgccagcacagaagagaaaacacaagaaatacaagcatgtcaacgataagaaagtgctgaaagaaatcctcatagcatag

SEQ ID NO: 117 - DNA, 660 bp, synthetic construct
  source 1..660: mol_type = other DNA
atgtgcccgcagccggcaaggcttgaacccggcatgaatttcggcgtggtcttcgccgtcatcctctccctgcccctggcccgcctggagggggaccccatacccgaagatatttatgagattttgggtggcagctccgtgcgctccatcagtgacctccagcgtgccctgcggatagactccgtagaggaggacagctctagcctggacctgaatgcaactcagcccagccaaaaccatgtgtccctgtctcgagagaggcgaagccttgatgctctggcagcagcagagccagctgtcctcgccgagtgcaagacacggacggtggtctttgagatctcccgtgacatggtggacagcaccaatgccaacttcgtggtgtggccaccctgcgtggaggtgcagcggtgctccggctgctgcaacaaccgcaacgtgcagtgccgccccatgcagattcgcgtccggcatgtccaggtgaacaagattgagtttttccagaggaagccaatattcaaaaaagtcatcgtgcctttggaggaccacgtgcagtgccggtgcgaagtggtgtcccggccgccacccaggagcaaccgaccggcatcccgtgagcagagacgcttctcgccgtcattcaccacagccgccatctcccagtag

SEQ ID NO: 118 - DNA, 114 bp, synthetic construct
  source 1..114: mol_type = other DNA
atggcgaaaggtggcataatcgtcgcgatccttctccttatcgttatgcttgctatagagatattgcttttgataactcttatcattgctgtaacgtctggaggcagcgggtag

SEQ ID NO: 119 - DNA, 138 bp, synthetic construct
  source 1..138: mol_type = other DNA
atggcggattacaaggatgatgatgacaaaaaaggtggcataatcgtcgcgatccttctccttatcgttatgcttgctatagagatattgcttttgataactcttatcattgctgtaacgtctggaggcagcgggtag

SEQ ID NO: 120 - DNA, 5831 bp, synthetic construct
  source 1..5831: mol_type = other DNA
gcccctgcagccgaattatattatttttgccaaataatttttaacaaaagctctgaagtcttcttcatttaaattcttagatgatacttcatctggaaaattgtcccaattagtagcatcacgctgtgagtaagttctaaaccatttttttattgttgtattatctctaatcttactactcgatgagttttcggtattatctctatttttaacttggagcaggttccattcattgtttttttcatcatagtgaataaaatcaactgctttaacacttgtgcctgaacaccatatccatccggcgtaatacgactcactatagggagagcggccgcgtcgacatgcccgccgtgaccgtcgagaacccgctgacgctgccccgcgtatccgcacccgccgacgccgtcgcacgtcccgtgctcaccgtgaccaccgcgcccagcggtttcgagggcgagggcttcccggtgcgccgcgcgttcgccgggatcaactaccgccacctcgacccgttcatcatgatggaccagatgggtgaggtggagtacgcgcccggggagcccaagggcacgccctggcacccgcaccgcggcttcgagaccgtgacctacatcgtcgacgcggccgccagatcttccggatggctcgagtttttcagcaagatatgataagatacattgatgagtttggacaaaccacaactagaatgcagtgaaaaaaatgctttatttgtgaaatttgtgatgctattgctttatttgtaaccattataagctgcaataaacaagtttagttaacgcatgatacaaaggcattaaagcagcgtatccacatagcgtaaaaggagcaacatagttaagaataccagtcaatctttcacaaattttgtaatccagaggttgatttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatagggccgggattctcctccacgtcaccgcatgttagaagacttcctctgccctcgcgagatccggtggagccgggtccggcggtgccgtccacggcagaattggacgactgagcgcgggatctggcgaaggcgatgggggtcttgaaggcgtgctggtactccacgatgcccagctcggtgttgctgtgcagctcctccacgcggcggaaggcgaacatggggcccccgttctgcaggatgctggggtggatggcgctcttgaagtgcatgtggctgtccaccacgaagctgtagtagccgccgtcgcgcaggctgaaggtgcgggcgaagctgcccaccagcacgttatcgcccatggggtgcaggtgctccacggtggcgttgctgcggatgatcttgtcggtgaagatcacgctgtcctcggggaagccggtgcccaccaccttgaagtcgccgatcacgcggccggcctcgtagcggtagctgaagctcacgtgcagcacgccgccgtcctcgtacttctcgatgcgggtgttggtgtagccgccgttgttgatggcgtgcaggaaggggttctcgtagccgctggggtaggtgccgaagtggtagaagccgtagcccatcacgtggctcagcaggtaggggctgaaggtcagggcgcctttggtgctcttcatcttgttggtcatgcggccctgcttgggggtgccctctccgccgcccaccagctcgaactccacgccgttcagggtgccggtgatgcggcactcgatctccatggcgggcaggccgctctcgtcgctctccatggtggcgtctagcgtaggcgccggtcacagcttggatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcacccgttcaattgccgacccctccccccaacttctcggggactgtgggcgatgtgcgctctgcccactgacgggcaccggagcgatcgcagatccttatctttctagaaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagcagctgggaagggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctggatccgatatcggtaccgctagcatcgatcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttgtttaaacctcctgtgtgaaattattatccgctcataattccacacattatacgagccggaagcataaagtgtaaagcctggggtgcctaatgagtgagctaactcacattaattgcgttgcgctcactgccaattgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcc

SEQ ID NO: 121 - DNA, 6079 bp, synthetic construct
  source 1..6079: mol_type = other DNA
gcccctgcagccgaattatattatttttgccaaataatttttaacaaaagctctgaagtcttcttcatttaaattcttagatgatacttcatctggaaaattgtcccaattagtagcatcacgctgtgagtaagttctaaaccatttttttattgttgtattatctctaatcttactactcgatgagttttcggtattatctctatttttaacttggagcaggttccattcattgtttttttcatcatagtgaataaaatcaactgctttaacacttgtgcctgaacaccatatccatccggcgtaatacgactcactatagggagagcggccgccagatcttccggatggctcgagtttttcagcaagatcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgcatatgtcaggcaccgggcttgcgggtcatgcaccaggtgcgcggtccttcgggcacctcgacgtcggcggtgacggtgaagccgagccgctcgtagaaggggaggttgcggggcgcggatgtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcataggaccggggttttcttccacgtctcctgcttgctttaacagagagaagttcgtggcaccggatcctccagcgcctgtgctatgtctgccctcagctctctcatattgttccacgatggtgtagtccccattatggctgataatgtcgagtttaatatcagtcatgtaggcgccaggcagctgcacttgtttcttggccttgtaggttgttttgacctcggcatcgtagtgtcctccgtctttgagtttcagtctcattttaatttcgcctttcagagcgccatcctcaggatacattctctcggtggaggcttcccatcccattgtttttttttgcatgacagggccatcggaggggaagttggttcctctgagtttcaccttataaataaactctccgtcctggagggtgctatcttgtgtgactgtcaccacgcctccgtcctcgaagttcatgaatctctcccacttgaagccttcagggaaggagagcttcagatagtcagggatgtcggcagggtgtttaacataggctttgcttccgtactggaactgagggctcagaatatcccaggagaaggggaggggtcctccttttgtgaccttgagcttagcggtttgtgtgccctcgtaaggccggccttctccttctccctcaatctcgaactcgtgtccgttgacgcttccctccatcttgaccttaaatctcataaactctttgatgacatcctcggagctggccatggtggcgatcgataggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccacccttcccagctgctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgctaccggtggatgtggaatgtgtgcgaggccagaggccacttgtgtagcgccaagtgccagcggggctgctaaagcgcatgctccagactgccttgggaaaagcgcctcccctacccggtagaattatctttctagaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaattcgatatcggtaccgctagccctaggacgcgtcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttatcgttcttcttttattctctcaagattttcaggctgtatattaaaacttatattaagaactatgctaaccacctcatcaggaaccgttgtaggtggcgtgggttttcttggcaatcgactctcatgaaaactacgagctaaatattcaatatgttcctcttgaccaactttattctgcattttttttgaacgaggtttagagcaagcttgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacaagcttcaggaaactgagacaggaattttattaaaaatttaaattttgaagaaagttcagggttaatagcatccattttttgctttgcaagttcctcagcattcttaacaaaagacgtctcttttgacatgtttaaagtttaaacctcctgtgtgaaattattatccgctcataattccacacattatacgagccggaagcataaagtgtaaagcctggggtgcctaatgagtgagctaactcacattaattgcgttgcgctcactgccaattgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcc

SEQ ID NO: 122 - DNA, 6118 bp, synthetic construct
  source 1..6118: mol_type = other DNA
gcccctgcagccgaattatattatttttgccaaataatttttaacaaaagctctgaagtcttcttcatttaaattcttagatgatacttcatctggaaaattgtcccaattagtagcatcacgctgtgagtaagttctaaaccatttttttattgttgtattatctctaatcttactactcgatgagttttcggtattatctctatttttaacttggagcaggttccattcattgtttttttcatcatagtgaataaaatcaactgctttaacacttgtgcctgaacaccatatccatccggcgtaatacgactcactatagggagagcggccgccagatcttccggatggctcgagtttttcagcaagatcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgcatatgtcaggcaccgggcttgcgggtcatgcaccaggtgcgcggtccttcgggcacctcgacgtcggcggtgacggtgaagccgagccgctcgtagaaggggaggttgcggggcgcggatgtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcataggaccggggttttcttccacgtctcctgcttgctttaacagagagaagttcgtggcaccggatcccttgtacagctcgtccatgccgagagtgatcccggcggcggtgcggaactccagcaggaccatgtgatcgcgcttctcgttggggtctttgctcagcacggactgggtgctcaggtagtggctgtcgggcagcagcacggggccgtcgccgatgggggtgttctgctggtagtggtcggcgagctgcacgctgccgtcctccacgttgtggcggatcttgaagttcaccttgatgccgttcttctgcttgacggccatgatatagatgttgtggctgttgaagttgtactccagcttgtgccccaggatgttgccgtcctccttgaagtcgacgcccttcagctcgatgcggttcactagggtgtcgccctcgaacttcacctcggcgcgggtcttgtaggtgccgtcgtccttgaagaagatggtgcgctcctggacgtagccttcgggcatggcggacttgaagaagtcgtgctgcttcatgtggtcggggtagcgggcgaagcactgcacgccgtggctcagggtggtcacgagggtgggccagggcacgggcagcttgccggtggtgcagatgaacttcagggtcagcttgccgttggtggcatcgccctcgccctcgcccctcacgctgaacttgtggccgtttacgtcgccgtccagctcgaccaggatgggcaccaccccggtgaacagctcctcgcccttgctcaccatggtggcgatcgataggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccacccttcccagctgctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgctaccggtggatgtggaatgtgtgcgaggccagaggccacttgtgtagcgccaagtgccagcggggctgctaaagcgcatgctccagactgccttgggaaaagcgcctcccctacccggtagaattatctttctagaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaattcgatatcggtaccgctagccctaggacgcgtcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttatcgttcttcttttattctctcaagattttcaggctgtatattaaaacttatattaagaactatgctaaccacctcatcaggaaccgttgtaggtggcgtgggttttcttggcaatcgactctcatgaaaactacgagctaaatattcaatatgttcctcttgaccaactttattctgcattttttttgaacgaggtttagagcaagcttgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacaagcttcaggaaactgagacaggaattttattaaaaatttaaattttgaagaaagttcagggttaatagcatccattttttgctttgcaagttcctcagcattcttaacaaaagacgtctcttttgacatgtttaaagtttaaacctcctgtgtgaaattattatccgctcataattccacacattatacgagccggaagcataaagtgtaaagcctggggtgcctaatgagtgagctaactcacattaattgcgttgcgctcactgccaattgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcc

SEQ ID NO: 123 - protein, 158 aa, synthetic construct
MAAMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCATDECFFFERLESNNYNTYRSRKYSSWYVALKRTGQYKLGPKTGPGQKAILFLPMSAKS

SEQ ID NO: 124 - protein, 155 aa, synthetic construct [sequence omitted]

SEQ ID NO: 125 - protein, 70 aa, synthetic construct
GPETLCGAELVDALQFVCGDRGFYFSKPTGYGSSSRRLHHKGIVDECCFQSCDLRRLEMYCAPIKPPKSA

SEQ ID NO: 126 - protein, 70 aa, synthetic construct; identical to SEQ ID NO: 125

SEQ ID NO: 127 - protein, 70 aa, synthetic construct; identical to SEQ ID NO: 125

SEQ ID NO: 128 - protein, 70 aa, synthetic construct
GPETLCGAELVDALQFVCGPRGFYFSKPTGYGSSIRRLHHKGIVDECCFQSCDLRRLEMYCAPIKPTKAA

SEQ ID NO: 129 - protein, 251 aa, synthetic construct
MCPQPARLEMNRCWALFLSLCCYLRLVSAEGDPIPEELYEMLSDHSIRSFDDLQRLLHGDSVEEDGAELDLNXTRSHSGGELESLSRGRRSLGSXTIAEPAVIAECKTRTEVFEISRRLIDRTNANFLVWPPCVEVQRCSGCCNNRNVQCRPTQVQXRXVQVXKIEIVRKKPIFKKATVTLEDHLACRCETVXAXRPVTRXPGSSQEQRXAXTPQTRVTIRTVRVRRPPKGKHRKFKHTHDKTALKETLGA

SEQ ID NO: 130 - protein, 241 aa, synthetic construct
MNRCWALFLSLCCYLRLVSAEGDPIPEELYKMLSDHSIRSFDDLQRLLHGDSVDEDGAELDLNLTRSHSGGELESLSRGRRSLGSPTVAAEPAVIAECKTRTEVFEISRRLIDRTNANFLVWPPCVEVQRCSGCCNNRNVQCRPTQVQDRKVQVKKIEIVRKKKIFKKATVTLVDHLACRCETVVARAVTRTPGSSQEQRARTPQTRVTIRTVRVRRPPKGKHRKFKHTHDKTALKETLGA

SEQ ID NO: 131 - protein, 241 aa, synthetic construct
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA